SEQUENCE LISTING (43 sequences)

SEQ ID NO: 1 - protein, 13 aa, Artificial Sequence
  Description of Artificial Sequence Synthetic peptide
GRKKRRQRRRPPQ

SEQ ID NO: 2 - protein, 16 aa, Artificial Sequence
  Description of Artificial Sequence Synthetic peptide
RQIKIWFQNRRMKWKK

SEQ ID NO: 3 - protein, 18 aa, Artificial Sequence
  Description of Artificial Sequence Synthetic peptide
KLALKLALKALKAALKLA

SEQ ID NO: 4 - protein, 26 aa, Artificial Sequence
  Description of Artificial Sequence Synthetic peptide
  MOD_RES (6)..(6): S or A
GWTLNXGYLLGKINLKALAALAKKIL

SEQ ID NO: 5 - protein, 17 aa, Artificial Sequence
  Description of Artificial Sequence Synthetic peptide
NAKTRRHERRRKLAIER

SEQ ID NO: 6 - protein, 8 aa, Artificial Sequence
  Description of Artificial Sequence Synthetic peptide
RRRRRRRR

SEQ ID NO: 7 - protein, 9 aa, Artificial Sequence
  Description of Artificial Sequence Synthetic peptide
RRRRRRRRR

SEQ ID NO: 8 - protein, 17 aa, Artificial Sequence
  Description of Artificial Sequence Synthetic peptide
GALFLGFLGAAGSTMGA

SEQ ID NO: 9 - protein, 21 aa, Artificial Sequence
  Description of Artificial Sequence Synthetic peptide
KETWWETWWTEWSQPKKKRKV

SEQ ID NO: 10 - protein, 18 aa, Artificial Sequence
  Description of Artificial Sequence Synthetic peptide
LLIILRRRIRKQAHAHSK

SEQ ID NO: 11 - protein, 16 aa, Artificial Sequence
  Description of Artificial Sequence Synthetic peptide
YTAIAWVKAFIRKLRK

SEQ ID NO: 12 - protein, 17 aa, Artificial Sequence
  Description of Artificial Sequence Synthetic peptide
IAWVKAFIRKLRKGPLG

SEQ ID NO: 13 - protein, 22 aa, Artificial Sequence
  Description of Artificial Sequence Synthetic peptide
MVTVLFRRLRIRRACGPPRVRV

SEQ ID NO: 14 - protein, 20 aa, Artificial Sequence
  Description of Artificial Sequence Synthetic peptide
GLWRALWRLLRSLWRLLWRA

SEQ ID NO: 15 - protein, 24 aa, Artificial Sequence
  Description of Artificial Sequence Synthetic peptide
RRRRRRRQIKIWFQNRRMKWKKGG

SEQ ID NO: 16 - protein, 23 aa, Artificial Sequence
  Description of Artificial Sequence Synthetic peptide
  MOD_RES (2)..(2): 6-aminohexanoic acid
  MOD_RES (5)..(5): 6-aminohexanoic acid
  MOD_RES (8)..(8): 6-aminohexanoic acid
RXRRXRRXRIKILFQNRRMKWKK

SEQ ID NO: 17 - protein, 24 aa, Artificial Sequence
  Description of Artificial Sequence Synthetic peptide
  MOD_RES (2)..(2): 6-aminohexanoic acid
  MOD_RES (5)..(5): 6-aminohexanoic acid
  MOD_RES (8)..(8): 6-aminohexanoic acid
  MOD_RES (11)..(11): D-Lysine
  MOD_RES (17)..(17): D-Arginine
  MOD_RES (24)..(24): Beta-alanine
RXRRXRRXRIKILFQNRRMKWHKA

SEQ ID NO: 18 - protein, 24 aa, Artificial Sequence
  Description of Artificial Sequence Synthetic peptide
  MOD_RES (2)..(2): 6-aminohexanoic acid
  MOD_RES (5)..(5): 6-aminohexanoic acid
  MOD_RES (8)..(8): 6-aminohexanoic acid
  MOD_RES (17)..(17): D-Arginine
  MOD_RES (24)..(24): Beta-alanine
RXRRXRRXRIHILFQNRRMKWHKA

SEQ ID NO: 19 - protein, 22 aa, Artificial Sequence
  Description of Artificial Sequence Synthetic peptide
  MOD_RES (2)..(2): 6-aminohexanoic acid
  MOD_RES (5)..(5): Beta-alanine
  MOD_RES (8)..(8): 6-aminohexanoic acid
  MOD_RES (16)..(16): 6-aminohexanoic acid
  MOD_RES (18)..(18): Beta-alanine
  MOD_RES (20)..(20): 6-aminohexanoic acid
  MOD_RES (22)..(22): Beta-alanine
RXRRARRXRILFQYRXRARXRA

SEQ ID NO: 20 - protein, 22 aa, Artificial Sequence
  Description of Artificial Sequence Synthetic peptide
  MOD_RES (2)..(2): 6-aminohexanoic acid
  MOD_RES (5)..(5): Beta-alanine
  MOD_RES (8)..(8): 6-aminohexanoic acid
  MOD_RES (16)..(16): 6-aminohexanoic acid
  MOD_RES (18)..(18): 6-aminohexanoic acid
  MOD_RES (20)..(20): 6-aminohexanoic acid
  MOD_RES (22)..(22): Beta-alanine
RXRRARRXRILFQYRXRXRXRA

SEQ ID NO: 21 - protein, 17 aa, Artificial Sequence
  Description of Artificial Sequence Synthetic peptide
  MOD_RES (2)..(2): 6-aminohexanoic acid
  MOD_RES (5)..(5): 6-aminohexanoic acid
  MOD_RES (13)..(13): 6-aminohexanoic acid
  MOD_RES (16)..(16): 6-aminohexanoic acid
RXRRXRILFQYRXRRXR

SEQ ID NO: 22 - protein, 22 aa, Artificial Sequence
  Description of Artificial Sequence Synthetic peptide
  MOD_RES (2)..(2): Beta-alanine
  MOD_RES (5)..(5): 6-aminohexanoic acid
  MOD_RES (8)..(8): Beta-alanine
  MOD_RES (16)..(16): Beta-alanine
  MOD_RES (18)..(18): 6-aminohexanoic acid
  MOD_RES (20)..(20): Beta-alanine
  MOD_RES (22)..(22): Beta-alanine
RARRXRRARILFQYRARXRARA

SEQ ID NO: 23 - protein, 22 aa, Artificial Sequence
  Description of Artificial Sequence Synthetic peptide
  MOD_RES (2)..(2): Beta-alanine
  MOD_RES (5)..(5): 6-aminohexanoic acid
  MOD_RES (8)..(8): Beta-alanine
  MOD_RES (16)..(16): 6-aminohexanoic acid
  MOD_RES (18)..(18): Beta-alanine
  MOD_RES (20)..(20): 6-aminohexanoic acid
  MOD_RES (22)..(22): Beta-alanine
RARRXRRARILFQYRXRARXRA

SEQ ID NO: 24 - protein, 21 aa, Artificial Sequence
  Description of Artificial Sequence Synthetic peptide
  MOD_RES (2)..(2): Beta-alanine
  MOD_RES (5)..(5): 6-aminohexanoic acid
  MOD_RES (8)..(8): Beta-alanine
  MOD_RES (16)..(16): 6-aminohexanoic acid
  MOD_RES (19)..(19): 6-aminohexanoic acid
  MOD_RES (21)..(21): Beta-alanine
RARRXRRARILFQYRXRRXRA

SEQ ID NO: 25 - protein, 21 aa, Artificial Sequence
  Description of Artificial Sequence Synthetic peptide
  MOD_RES (2)..(2): Beta-alanine
  MOD_RES (5)..(5): 6-aminohexanoic acid
  MOD_RES (8)..(8): Beta-alanine
  MOD_RES (16)..(16): 6-aminohexanoic acid
  MOD_RES (18)..(18): Beta-alanine
  MOD_RES (20)..(20): 6-aminohexanoic acid
  MOD_RES (21)..(21): Beta-alanine
RARRXRRARILFQYRXRARXA

SEQ ID NO: 26 - protein, 21 aa, Artificial Sequence
  Description of Artificial Sequence Synthetic peptide
  MOD_RES (2)..(2): 6-aminohexanoic acid
  MOD_RES (5)..(5): Beta-alanine
  MOD_RES (8)..(8): 6-aminohexanoic acid
  MOD_RES (16)..(16): 6-aminohexanoic acid
  MOD_RES (19)..(19): 6-aminohexanoic acid
  MOD_RES (21)..(21): Beta-alanine
RXRRARRXRILFQYRXRRXRA

SEQ ID NO: 27 - protein, 21 aa, Artificial Sequence
  Description of Artificial Sequence Synthetic peptide
  MOD_RES (2)..(2): 6-aminohexanoic acid
  MOD_RES (5)..(5): Beta-alanine
  MOD_RES (8)..(8): 6-aminohexanoic acid
  MOD_RES (16)..(16): 6-aminohexanoic acid
  MOD_RES (18)..(18): Beta-alanine
  MOD_RES (20)..(20): 6-aminohexanoic acid
  MOD_RES (21)..(21): Beta-alanine
RXRRARRXRILFQYRXRARXA

SEQ ID NO: 28 - protein, 22 aa, Artificial Sequence
  Description of Artificial Sequence Synthetic peptide
  MOD_RES (2)..(2): 6-aminohexanoic acid
  MOD_RES (5)..(5): Beta-alanine
  MOD_RES (8)..(8): 6-aminohexanoic acid
  MOD_RES (16)..(16): 6-aminohexanoic acid
  MOD_RES (18)..(18): Beta-alanine
  MOD_RES (20)..(20): 6-aminohexanoic acid
  MOD_RES (22)..(22): Beta-alanine
RXRRARRXRYQFLIRXRARXRA

SEQ ID NO: 29 - protein, 22 aa, Artificial Sequence
  Description of Artificial Sequence Synthetic peptide
  MOD_RES (2)..(2): 6-aminohexanoic acid
  MOD_RES (5)..(5): Beta-alanine
  MOD_RES (8)..(8): 6-aminohexanoic acid
  MOD_RES (16)..(16): 6-aminohexanoic acid
  MOD_RES (18)..(18): Beta-alanine
  MOD_RES (20)..(20): 6-aminohexanoic acid
  MOD_RES (22)..(22): Beta-alanine
RXRRARRXRIQFLIRXRARXRA

SEQ ID NO: 30 - protein, 21 aa, Artificial Sequence
  Description of Artificial Sequence Synthetic peptide
  MOD_RES (2)..(2): 6-aminohexanoic acid
  MOD_RES (5)..(5): Beta-alanine
  MOD_RES (8)..(8): 6-aminohexanoic acid
  MOD_RES (15)..(15): 6-aminohexanoic acid
  MOD_RES (17)..(17): Beta-alanine
  MOD_RES (19)..(19): 6-aminohexanoic acid
  MOD_RES (21)..(21): Beta-alanine
RXRRARRXRQFLIRXRARXRA

SEQ ID NO: 31 - protein, 20 aa, Artificial Sequence
  Description of Artificial Sequence Synthetic peptide
  MOD_RES (2)..(2): 6-aminohexanoic acid
  MOD_RES (5)..(5): Beta-alanine
  MOD_RES (8)..(8): 6-aminohexanoic acid
  MOD_RES (14)..(14): 6-aminohexanoic acid
  MOD_RES (16)..(16): Beta-alanine
  MOD_RES (18)..(18): 6-aminohexanoic acid
  MOD_RES (20)..(20): Beta-alanine
RXRRARRXRQFLRXRARXRA

SEQ ID NO: 32 - protein, 21 aa, Artificial Sequence
  Description of Artificial Sequence Synthetic peptide
  MOD_RES (2)..(2): 6-aminohexanoic acid
  MOD_RES (5)..(5): Beta-alanine
  MOD_RES (8)..(8): 6-aminohexanoic acid
  MOD_RES (15)..(15): 6-aminohexanoic acid
  MOD_RES (17)..(17): Beta-alanine
  MOD_RES (19)..(19): 6-aminohexanoic acid
  MOD_RES (21)..(21): Beta-alanine
RXRRARRXYRFLIRXRARXRA

SEQ ID NO: 33 - protein, 22 aa, Artificial Sequence
  Description of Artificial Sequence Synthetic peptide
  MOD_RES (2)..(2): 6-aminohexanoic acid
  MOD_RES (5)..(5): Beta-alanine
  MOD_RES (8)..(8): 6-aminohexanoic acid
  MOD_RES (16)..(16): 6-aminohexanoic acid
  MOD_RES (18)..(18): Beta-alanine
  MOD_RES (20)..(20): 6-aminohexanoic acid
  MOD_RES (22)..(22): Beta-alanine
RXRRARRXRFQILYRXRARXRA

SEQ ID NO: 34 - protein, 21 aa, Artificial Sequence
  Description of Artificial Sequence Synthetic peptide
  MOD_RES (2)..(2): 6-aminohexanoic acid
  MOD_RES (5)..(5): Beta-alanine
  MOD_RES (8)..(8): 6-aminohexanoic acid
  MOD_RES (15)..(15): 6-aminohexanoic acid
  MOD_RES (17)..(17): Beta-alanine
  MOD_RES (19)..(19): 6-aminohexanoic acid
  MOD_RES (21)..(21): Beta-alanine
RXRRARRXYRFRLIXRARXRA

SEQ ID NO: 35 - protein, 21 aa, Artificial Sequence
  Description of Artificial Sequence Synthetic peptide
  MOD_RES (2)..(2): 6-aminohexanoic acid
  MOD_RES (5)..(5): Beta-alanine
  MOD_RES (8)..(8): 6-aminohexanoic acid
  MOD_RES (15)..(15): 6-aminohexanoic acid
  MOD_RES (17)..(17): Beta-alanine
  MOD_RES (19)..(19): 6-aminohexanoic acid
  MOD_RES (21)..(21): Beta-alanine
RXRRARRXILFRYRXRARXRA

SEQ ID NO: 36 - protein, 13 aa, Artificial Sequence
  Description of Artificial Sequence Synthetic peptide
  MOD_RES (11)..(11): 6-aminohexanoic acid
  MOD_RES (12)..(12): L-bis-homopropargylglycine
RRLSYSRRRFXGG

SEQ ID NO: 37 - protein, 19 aa, Artificial Sequence
  Description of Artificial Sequence Synthetic peptide
  MOD_RES (17)..(17): 6-aminohexanoic acid
  MOD_RES (18)..(18): L-bis-homopropargylglycine
RRLSYSRRRFPFVYLIXGG

SEQ ID NO: 38 - DNA, 12 bp, Artificial Sequence
  Description of Artificial Sequence Synthetic oligonucleotide
gaagaagaagaa

SEQ ID NO: 39 - protein, 6 aa, Artificial Sequence
  Description of Artificial Sequence Synthetic peptide
  MOD_RES (2)..(2): 6-aminohexanoic acid
  MOD_RES (5)..(5): Beta-alanine
RXRRAR

SEQ ID NO: 40 - protein, 5 aa, Artificial Sequence
  Description of Artificial Sequence Synthetic peptide
ILFQY

SEQ ID NO: 41 - protein, 4 aa, Artificial Sequence
  Description of Artificial Sequence Synthetic peptide
QFLY

SEQ ID NO: 42 - protein, 8 aa, Artificial Sequence
  Description of Artificial Sequence Synthetic peptide
  MOD_RES (2)..(2): 6-aminohexanoic acid
  MOD_RES (4)..(4): Beta-alanine
  MOD_RES (6)..(6): 6-aminohexanoic acid
  MOD_RES (8)..(8): Beta-alanine
RXRARXRA

SEQ ID NO: 43 - protein, 10 aa, Artificial Sequence
  Description of Artificial Sequence Synthetic peptide
  SITE (1)..(10): This sequence may encompass 5-10 residues
RRRRRRRRRR

(19) United States
(12) Patent Application Publication
(10) Pub. No.: US 2024/0124491 A1
(43) Pub. Date: Apr. 18, 2024

(54) METHODS AND COMPOUNDS FOR TREATING FRIEDREICH'S ATAXIA

(71) Applicant: Design Therapeutics, Inc., Carlsbad, CA (US)

(72) Inventors: Aseem ANSARI, Madison, WI (US); Abhijit BHAT, Carlsbad, CA (US); Sean J. JEFFRIES, Carlsbad, CA (US); Pratik SHAH, Carlsbad, CA (US); Chengzhi ZHANG, Carlsbad, CA (US)

(21) Appl. No.: 18/260,549

(22) PCT Filed: Jan. 7, 2022
(86) PCT No.: PCT/US2022/011560

§ 371 Date: Jul. 6, 2023

Related U.S. Application Data
(60) Provisional application No. 63/135,427, filed on Jan. 8, 2021.

(51) Int. Cl. C07D 519/00; A61K 45/06; A61P 25/14; C07D 401/14; C07D 471/04; C07D 487/04; C07D 495/14; C07D 498/04

(52) U.S. Cl. CPC C07D 519/00; A61K 45/06; A61P 25/14; C07D 401/14; C07D 471/04; C07D 487/04; C07D 495/14; C07D 498/04

(57) ABSTRACT

The present disclosure relates to compounds and methods for modulating the expression of fxn, and treating diseases and conditions in which fxn plays an active role. The compound can be a transcription modulator molecule having a first terminus, a second terminus, and oligomeric backbone, wherein: a) the first terminus comprises a DNA-binding moiety capable of noncovalently binding to a trinucleotide repeat sequence GAA; b) the second terminus comprises a protein-binding moiety binding to a regulatory molecule that modulates an expression of a gene comprising the nucleotide repeat sequence GAA; and c) the oligomeric backbone comprising a linker between the first terminus and the second terminus.

### CROSS REFERENCE

This application claims the benefit of U.S. Application No. 63/135,427, filed Jan. 8, 2021, which is hereby incorporated by reference in its entirety.

### FIELD OF THE DISCLOSURE

Disclosed herein are new chimeric heterocyclic polyamide compounds and compositions and their application as pharmaceuticals for the treatment of disease. Methods to modulate the expression of fxn in a human or animal subject are also provided for the treatment diseases such as Friedreich's ataxia.

### BACKGROUND OF THE DISCLOSURE

The disclosure relates to the treatment of inherited genetic diseases characterized by overproduction of mRNA.

Friedreich's ataxia (“FA” or “FRDA”) is an autosomal recessive neurodegenerative disorder caused by mutations in the fxn gene, which encodes the protein frataxin (“FXN”), an iron-binding mitochondrial protein involved in electron transport and metabolism. In most subjects with FA, a GAA trinucleotide repeat (from about 66 to over 1000 trinucleotides) is included in the first intron of fxn, and this hyperexpansion is responsible for the observed pathology. Hyperexpansion of the GAA repeats results in reduced expression of FXN.

Friedreich's ataxia is characterized by progressive degradation of the nervous system, particularly sensory neurons. In addition, cardiomyocytes and pancreatic beta cells are susceptible to frataxin depletion. Symptoms usually present by age 18; however, later diagnoses of FA are not uncommon. FA patients develop neurodegeneration of the large sensory neurons and spinocerebellar tracts, as well as cardiomyopathy and diabetes mellitus. Clinical symptoms of FA include ataxia, gait ataxia, muscle weakness, loss of upper body strength, loss of balance, lack of reflexes in lower limbs and tendons, loss of sensation, particularly to vibrations, impairment of position sense, impaired perception of temperature, touch, and pain, hearing and vision impairment, including distorted color vision and involuntary eye movements, irregular foot configuration, including pes cavus and inversion, hearing impairment, dysarthria, dysphagia, impaired breathing, scoliosis, diabetes, intolerance to glucose and carbohydrates, cardiac dysfunctions including hypertrophic cardiomyopathy, arrhythmia, myocardial fibrosis, and cardiac failure. Currently there is no cure for FA, with medical treatments being limited to surgical intervention for the spine and the heart, as well as therapy to assist with balance, coordination, motion, and speech.

### SUMMARY OF THE DISCLOSURE

This disclosure utilizes regulatory molecules present in cell nuclei that control gene expression. Eukaryotic cells provide several mechanisms for controlling gene replication, transcription, and/or translation. Regulatory molecules that are produced by various biochemical mechanisms within the cell can modulate the various processes involved in the conversion of genetic information to cellular components. Several regulatory molecules are known to modulate the production of mRNA and, if directed to fxn, could modulate the production of fxn mRNA that causes Friedreich's ataxia, and thus, reverse the progress of the disease.

The disclosure provides compounds and methods for recruiting a regulatory molecule into close proximity to fxn. The compounds disclosed herein contain: (a) a recruiting moiety that will bind to a regulatory molecule, linked to (b) a DNA binding moiety that will selectively bind to fxn. The compounds will counteract the expression of defective fxn in the following manner:
(1) The DNA binding moiety will bind selectively the characteristic GAA trinucleotide repeat sequence of fxn;
(2) The recruiting moiety, linked to the DNA binding moiety, will thus be held in proximity to fxn;
(3) The recruiting moiety, now in proximity to fxn, will recruit the regulatory molecule into proximity with the gene; and
(4) The regulatory molecule will modulate expression, and therefore counteract the production of defective fxn by direct interaction with the gene.

The mechanism set forth above will provide an effective treatment for Friedreich's ataxia, which is caused by the expression of defective fxn gene. Correction of the expression of the defective fxn gene thus represents a promising method for the treatment of Friedreich's ataxia.

The disclosure provides recruiting moieties that will bind to regulatory molecules. Small molecule inhibitors of regulatory molecules serve as templates for the design of recruiting moieties, since these inhibitors generally act via noncovalent binding to the regulatory molecules.

The disclosure further provides for DNA binding moieties that will selectively bind to one or more copies of the GAA trinucleotide repeat that is characteristic of the defective fxn gene. Selective binding of the DNA binding moiety to fxn, made possible due to the high GAA count associated with the defective fxn gene, will direct the recruiting moiety into proximity of the gene, and recruit the regulatory molecule into position to up-regulate gene transcription.

The DNA binding moiety will comprise a polyamide segment that will bind selectively to the target GAA sequence. Polyamides have been designed by Dervan (U.S. Pat. Nos. 9,630,950 and 8,524,899) and others that can selectively bind to selected DNA sequences. These polyamides sit in the minor groove of double helical DNA and form hydrogen bonding interactions with the Watson-Crick base pairs. Polyamides that selectively bind to particular DNA sequences can be designed by linking monoamide building blocks according to established chemical rules. One building block is provided for each DNA base pair, with each building block binding noncovalently and selectively to one of the DNA base pairs: A/T, T/A, G/C, and C/G. Following this guideline, trinucleotides will bind to molecules with three amide units, i.e. triamides. In general, these polyamides will orient in either direction of a DNA sequence, so that the 5′-GAA-3′ trinucleotide repeat sequence of fxn can be targeted by the polyamides selective either for GAA or for AAG. Furthermore, polyamides that bind to the complementary sequence, in this case, TTC or CTT, will also bind to the trinucleotide repeat sequence of fxn and can be employed as well.

In principle, longer DNA sequences can be targeted with higher specificity and/or higher affinity by combining a larger number of monoamide building blocks into longer polyamide chains. Ideally, the binding affinity for a polyamide would simply be equal to the sum of each individual monoamide/DNA base pair interaction. In practice, however, due to the geometric mismatch between the fairly rigid polyamide and DNA structures, longer polyamide sequences do not bind to longer DNA sequences as tightly as would be expected from a simple additive contribution. The geometric mismatch between longer polyamide sequences and longer DNA sequences induces an unfavorable geometric strain that subtracts from the binding affinity that would be otherwise expected.

The disclosure, therefore, provides DNA moieties that comprise triamides that are connected by flexible spacers. The spacers alleviate the geometric strain that would otherwise decrease binding affinity of a larger polyamide sequence.

Disclosed herein are compounds (i.e. transcription modulator molecules) the comprise a polyamide that can bind to one or more copies of the trinucleotide repeat sequence GAA, and can modulate the expression of the defective fxn gene. Treatment of a subject with these compounds may counteract the expression of the defective fxn gene, and this can reduce the occurrence, severity, and/or frequency of symptoms associated with Friedreich's ataxia. Certain compounds disclosed herein may provide higher binding affinity and/or selectivity than has been observed previously for this class of compound.

Other objects, features, and advantages of the compounds, methods, and compositions described herein will become apparent from the following detailed description. It should be understood, however, that the detailed description and the specific examples, while indicating specific embodiments, are given by way of illustration only, since various changes and modifications within the spirit and scope of the instant disclosure will become apparent to those skilled in the art from this detailed description.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE DISCLOSURE

The transcription modulator molecules described herein represent an interface of chemistry, biology and precision medicine in that the molecule can be programmed to regulate the expression of a target gene containing the nucleotide repeat GAA. The transcription modulator molecules contains DNA binding moieties that can selectively bind to one or more copies of the GAA hexanucleotide repeat that is characteristic of the defective fxn gene. The transcription modulator molecules also contains moieties that bind to regulatory proteins. The selective binding of the target gene can bring the regulatory protein into proximity to the target gene and thus downregulates transcription of the target gene. The molecules and compounds disclosed herein provide higher binding affinity and selectivity than has been observed previously for this class of compounds and can be more effective in treating diseases associated with the defective fxn gene.

The transcription modulator molecules described herein can recruit the regulatory molecule to modulate the expression of the defective fxn gene and effectively treat and/or and alleviate the symptoms associated with diseases such as Friedreich ataxia.

### Transcription Modulator Molecules

The transcription modulator molecules disclosed herein possess useful activity for modulating the transcription of a target gene having one or more GAA repeats (e.g., fxn), and may be used in the treatment or prophylaxis of a disease or condition in which the target gene (e.g., fxn) plays an active role. Thus, in broad aspect, certain embodiments also provide pharmaceutical compositions comprising one or more compounds disclosed herein together with a pharmaceutically acceptable carrier, as well as methods of making and using the compounds and compositions. Certain embodiments provide methods for modulating the expression of fxn. Other embodiments provide methods for treating a fxn-mediated disorder in a patient in need of such treatment, comprising administering to said patient a therapeutically effective amount of a compound or composition according to the present disclosure. Also provided is the use of certain compounds disclosed herein for use in the manufacture of a medicament for the treatment of a disease or condition ameliorated by the modulation of the expression of fxn.

Some embodiments relate to a transcription modulator molecule or compound having a first terminus, a second terminus, and oligomeric backbone, wherein: a) the first terminus comprises a DNA-binding moiety capable of noncovalently binding to a nucleotide repeat sequence GAA; b) the second terminus comprises a protein-binding moiety binding to a regulatory molecule that modulates an expression of a gene comprising the nucleotide repeat sequence GAA; and c) the oligomeric backbone comprising a linker between the first terminus and the second terminus. In some embodiments, the second terminus is a Brd4 binding moiety. In some embodiments, the second terminus is not a Brd4 binding moiety.

In certain embodiments, the compounds have structural Formula (I):

X-L-Y Formula (I)

or a salt thereof, wherein:
X comprises a is a recruiting moiety that is capable of noncovalent binding to a regulatory moiety within the nucleus;
Y comprises a DNA recognition moiety that is capable of noncovalent binding to one or more copies of the trinucleotide repeat sequence GAA; and
L is a linker.

Certain compounds disclosed herein may possess useful activity for modulating the transcription of fxn, and may be used in the treatment and/or prophylaxis of a disease or condition in which fxn plays an active role. Thus, in broad aspect, certain embodiments also provide pharmaceutical compositions comprising one or more compounds disclosed herein together with a pharmaceutically acceptable carrier, as well as methods of making and using the compounds and compositions. Certain embodiments provide methods for modulating the expression of fxn. Other embodiments provide methods for treating a fxn-mediated disorder in a patient in need of such treatment, comprising administering to said patient a therapeutically effective amount of a compound or composition according to the present disclosure. Also provided is the use of certain compounds disclosed herein for use in the manufacture of a medicament for the treatment of a disease or condition ameliorated by the modulation of the expression of fxn.

In certain embodiments, the regulatory molecule is chosen from a bromodomain-containing protein, a nucleosome remodeling factor (“NURF”), a bromodomain PHD finger transcription factor (“BPTF”), a ten-eleven translocation enzyme (“TET”), methylcytosine dioxygenase (“TET1”), a DNA demethylase, a helicase, an acetyltransferase, and a histone deacetylase (“HDAC”).

In some embodiments, the first terminus is Y, and the second terminus is X, and the oligomeric backbone is L.

In certain embodiments, the compounds have structural Formula (II):

X-L-(Y₅—Y₆—Y₇)ₙ—Y₀ Formula (II)

or a salt thereof, wherein:
X comprises a recruiting moiety that is capable of noncovalent binding to a regulatory molecule within the nucleus;
L is a linker;
Y₅, Y₆, and Y₇ are internal subunits, each of which comprises a moiety chosen from a heterocyclic ring or a C₁₋₆ straight chain aliphatic segment, and each of which is chemically linked to its two neighbors;
Y₀ is an end subunit which comprises a moiety chosen from a heterocyclic ring or a straight chain aliphatic segment, which is chemically linked to its single neighbor;
each subunit can noncovalently bind to an individual nucleotide in the GAA repeat sequence;
n is an integer between 1 and 200, inclusive; and
(Y₅—Y₆—Y₇)ₙ—Y₀ combine to form a DNA recognition moiety that is capable of noncovalent binding to one or more copies of the trinucleotide sequence GAA.

In certain embodiments, the compounds of structural Formula (II) comprise a subunit for each individual nucleotide in the GAA repeat sequence.

In certain embodiment, each internal subunit has an amino (—NH—) group and a carboxy (—CO—) group.

In certain embodiments, the compounds of structural Formula (II) comprise amide (—NHCO—) bonds between each pair of internal subunits.

In certain embodiments, the compounds of structural Formula (II) comprise an amide (—NHCO—) bond between L and the leftmost internal subunit.

In certain embodiments, the compounds of structural Formula (II) comprise an amide bond between the rightmost internal subunit and the end subunit.

In certain embodiments, each subunit comprises a moiety that is independently chosen from a heterocycle and an aliphatic chain.

In certain embodiments, the heterocycle is a monocyclic heterocycle. In certain embodiments, the heterocycle is a monocyclic 5-membered heterocycle. In certain embodiments, each heterocycle contains a heteroatom independently chosen from N, O, or S. In certain embodiments, each heterocycle is independently chosen from pyrrole, imidazole, thiazole, oxazole, thiophene, and furan.

In certain embodiments, the aliphatic chain is a C₁₋₆ straight chain aliphatic chain. In certain embodiments, the aliphatic chain has structural formula —(CH₂)ₘ—, for m chosen from 1, 2, 3, 4, and 5. In certain embodiments, the aliphatic chain is —CH₂CH₂—.

In certain embodiments, each subunit comprises a moiety independently chosen from

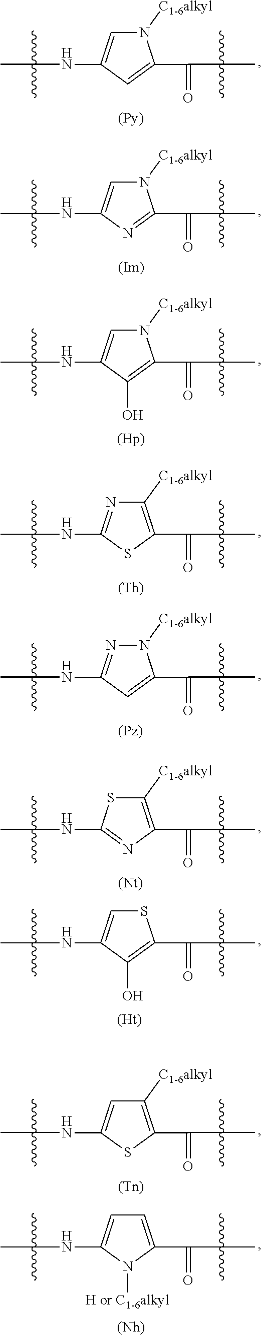

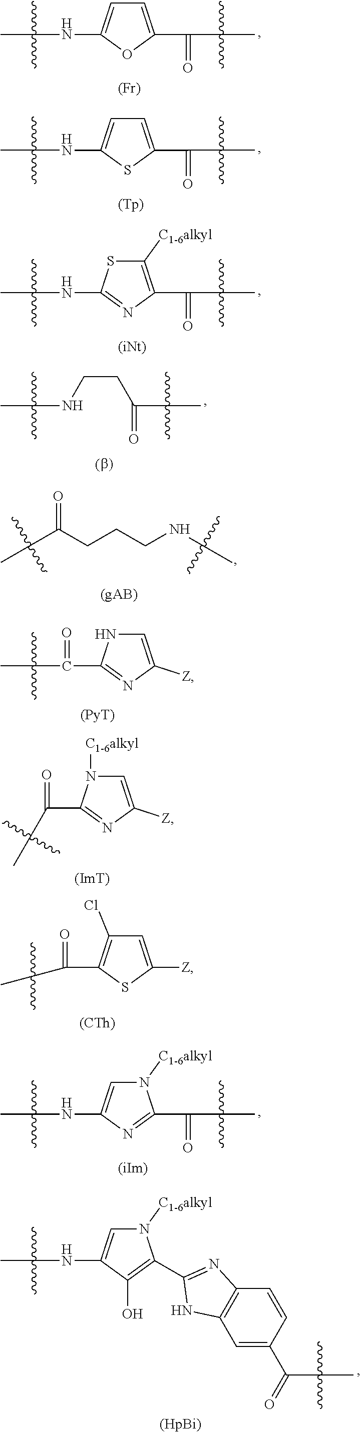

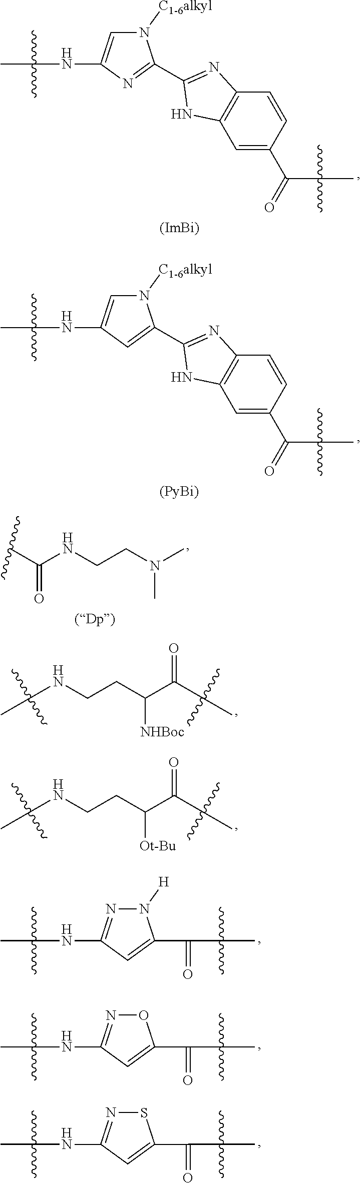

—NH-benzopyrazinylene-CO—, —NH-phenylene-CO—, —NH-pyridinylene-CO—, —NH-piperidinylene-CO—, —NH-pyrimidinylene-CO—, —NH-anthracenylene-CO—, —NH-quinolinylene-CO—, and

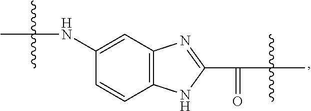

wherein Z is H, NH₂, C₁₋₆ alkyl, C₁₋₆ haloalkyl or C₁₋₆ alkyl-NH₂.

In some embodiments, Py is

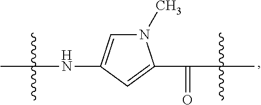 [SMILES: *Nc1cc(C(*)=O)n(C)c1]

### Im is

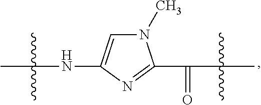 [SMILES: *Nc1cn(C)c(C(*)=O)n1]

### Hp is

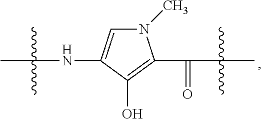 [SMILES: *Nc1cn(C)c(C(*)=O)c1O]

### Th is

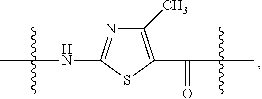 [SMILES: *Nc1nc(C)c(C(*)=O)s1]

### Pz is

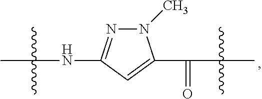 [SMILES: *Nc1cc(C(*)=O)n(C)n1]

### Nt is

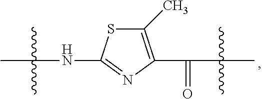 [SMILES: *Nc1nc(C(*)=O)c(C)s1]

### Tn is

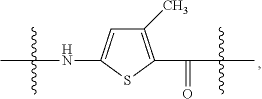 [SMILES: *Nc1cc(C)c(C(*)=O)s1]

### Nh is

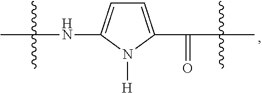 [SMILES: *Nc1ccc(C(*)=O)[nH]1]

iNt is

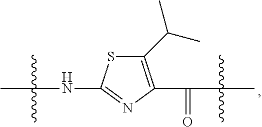 [SMILES: *Nc1nc(C(*)=O)c(C(C)C)s1]

iLm is

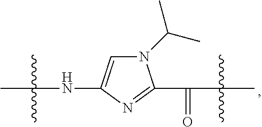 [SMILES: *Nc1cn(C(C)C)c(C(*)=O)n1]

### HpBi is

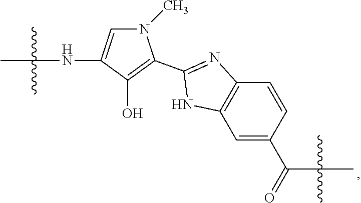 [SMILES: *Nc1cn(C)c(-c2nc3ccc(C(*)=O)cc3[nH]2)c1O]

### ImBi is

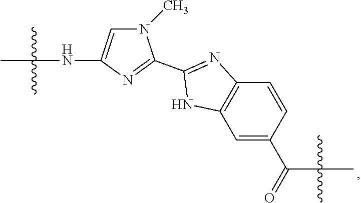 [SMILES: *Nc1cn(C)c(-c2nc3ccc(C(*)=O)cc3[nH]2)n1]

### PyBi is

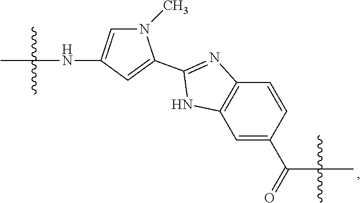 [SMILES: *Nc1cc(-c2nc3ccc(C(*)=O)cc3[nH]2)n(C)c1]

### Dp is

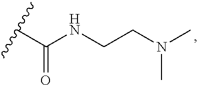 [SMILES: *C(=O)NCCN(C)C]

### —NH-benzopyrazinylene-CO— is

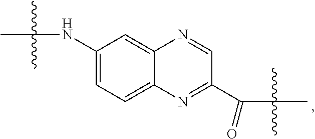 [SMILES: *Nc1ccc2nc(C(*)=O)cnc2c1]

### —NH-phenylene-CO— is

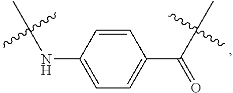 [SMILES: *Nc1ccc(C(*)=O)cc1]

### —NH-pyridinylene-CO— is

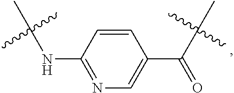 [SMILES: *Nc1ccc(C(*)=O)cn1]

### —NH-piperidinylene-CO— is

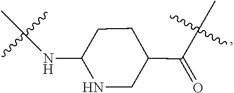 [SMILES: *NC1CCC(C(*)=O)CN1]

### —NH-pyrazinylene-CO— is

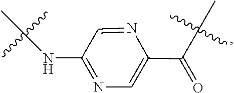 [SMILES: *Nc1cnc(C(*)=O)cn1]

### —NH-anthracenylene-CO— is

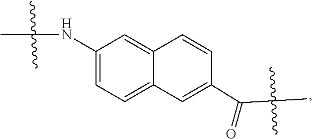 [SMILES: *Nc1ccc2cc(C(*)=O)ccc2c1]

### and —NH-quinolinylene-CO— is

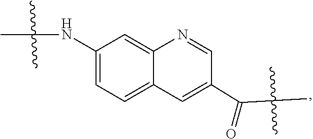 [SMILES: *Nc1ccc2cc(C(*)=O)cnc2c1]

In some embodiments, Py is

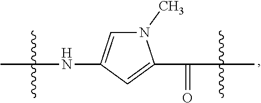 [SMILES: *Nc1cc(C(*)=O)n(C)c1]

### Im is

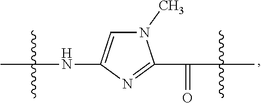 [SMILES: *Nc1cn(C)c(C(*)=O)n1]

### Hp is

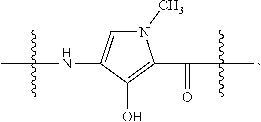 [SMILES: *Nc1cn(C)c(C(*)=O)c1O]

### Th is

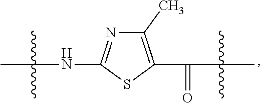 [SMILES: *Nc1nc(C)c(C(*)=O)s1]

### Pz is

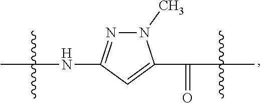 [SMILES: *Nc1cc(C(*)=O)n(C)n1]

### Nt is

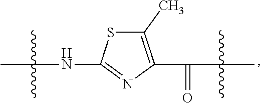 [SMILES: *Nc1nc(C(*)=O)c(C)s1]

### Tn is

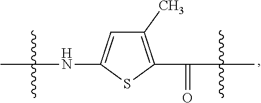 [SMILES: *Nc1cc(C)c(C(*)=O)s1]

### Nh is

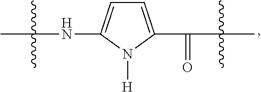 [SMILES: *Nc1ccc(C(*)=O)[nH]1]

iNt is

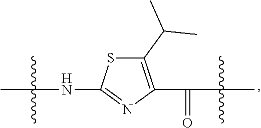 [SMILES: *Nc1nc(C(*)=O)c(C(C)C)s1]

and iLm is

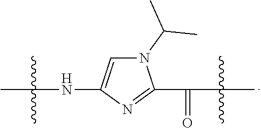 [SMILES: *Nc1cn(C(C)C)c(C(*)=O)n1]

In certain embodiments, n is between 1 and 100, inclusive. In certain embodiments, n is between 1 and 50, inclusive. In certain embodiments, n is between 1 and 20, inclusive. In certain embodiments, n is between 1 and 10, inclusive. In certain embodiments, n is between 1 and 5, inclusive. In certain embodiments, n is an integer between 1 and 3, inclusive. In certain embodiments, n is chosen from 1 and 2.

In certain embodiments, n is 1.

In certain embodiments, n is an integer between 1 and 5, inclusive.

In certain embodiments, n is an integer between 1 and 3, inclusive.

In certain embodiments, n is an integer between 1 and 2, inclusive.

In certain embodiments, n is 1.

In certain embodiments, L comprises a C₁₋₆ straight chain aliphatic segment.

In certain embodiments, L comprises (CH₂OCH₂)ₘ; and m is an integer between 1 to 20, inclusive.

In certain further embodiments, m is an integer between 1 to 10, inclusive. In certain further embodiments, m is an integer between 1 to 5, inclusive.

In certain embodiments, the compounds have structural Formula (III):

X-L-(Y₅—Y₆-Y₇)—(W—Y₅-Y₆—Y₇)ₙ—Y₀ Formula (III)

or a salt thereof, wherein:
X comprises a recruiting moiety that is capable of noncovalent binding to a regulatory molecule within the nucleus;
L is a linker;
Y₅, Y₆, and Y₇ are internal subunits, each of which comprises a moiety chosen from a heterocyclic ring or a C₁₋₆ straight chain aliphatic segment, and each of which is chemically linked to its two neighbors;
Y₀ is an end subunit which comprises a moiety chosen from a heterocyclic ring or a straight chain aliphatic segment, which is chemically linked to its single neighbor;
each subunit can noncovalently bind to an individual nucleotide in the GAA repeat sequence;
W is a spacer;
n is an integer between 1 and 200, inclusive; and
(Y₅—Y₆—Y₇)—(W—Y₅-Y₆—Y₇)ₙ—Y₀ combine to form a DNA recognition moiety that is capable of noncovalent binding to one or more copies of the trinucleotide repeat sequence GAA.

In certain embodiments, Y₅—Y₆-Y₇ is:

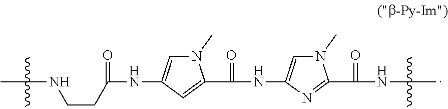 [SMILES: *NCCC(=O)Nc1cc(C(=O)Nc2cn(C)c(C(=O)N*)n2)n(C)c1]

In certain embodiments, Y₅—Y₆-Y₇ is:

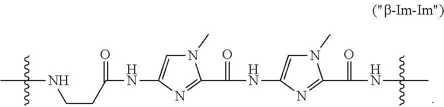 [SMILES: *NCCC(=O)Nc1cn(C)c(C(=O)Nc2cn(C)c(C(=O)N*)n2)n1]

In certain embodiments, Y₅—Y₆-Y₇ is Im-Py-β.

In certain embodiments, Y₅—Y₆-Y₇ is Im-Im-β.

In certain embodiments, each Y₅—Y₆-Y₇ is independently chosen from β-Py-Im and β-Im-Im.

In certain embodiments, at most one Y₅-Y₆—Y₇ is β-Im-Im.

In certain embodiments of the compound of structural Formula (III), n is between 1 and 100, inclusive. In certain embodiments of the compound of structural Formula (III), n is between 1 and 50, inclusive. In certain embodiments of the compound of structural Formula (III), n is between 1 and 20, inclusive. In certain embodiments of the compound of structural Formula (III), n is between 1 and 10, inclusive. In certain embodiments of the compound of structural Formula (III), n is between 1 and 5, inclusive. In certain embodiments of the compound of structural Formula (III), n is chosen from 1 and 2. In certain embodiments of the compound of structural Formula (III), n is 1.

In certain embodiments, the compounds have structural Formula (IV):

X-L-(Y₅—Y₆—Y₇)—V—(Y₈—Y₉—Y₁₀)—Y₀ Formula (IV)

or a salt thereof, wherein:
X comprises a recruiting moiety that is capable of noncovalent binding to a regulatory molecule within the nucleus;
Y₅, Y₆, Y₇, Y₈, Y₉, and Y₁₀ are internal subunits, each of which comprises a moiety chosen from a heterocyclic ring or a C₁₋₆ straight chain aliphatic segment, and each of which is chemically linked to its two neighbors;
Y₀ is an end subunit which comprises a moiety chosen from a heterocyclic ring or a straight chain aliphatic segment, which is chemically linked to its single neighbor;
each subunit can noncovalently bind to an individual nucleotide in the GAA repeat sequence;
L is a linker;
V is a turn component for forming a hairpin turn;
n is an integer between 1 and 200, inclusive; and
(Y₅—Y₆—Y)—V—(Y₈—Y₉-Y₁₀)—Y₀ combine to form a DNA recognition moiety that is capable of noncovalent binding to one or more copies of the trinucleotide repeat sequence GAA.

In certain embodiments of the compound of structural Formula (IV), n is between 1 and 100, inclusive. In certain embodiments of the compound of structural Formula (IV), n is between 1 and 50, inclusive. In certain embodiments of the compound of structural Formula (IV), n is between 1 and 20, inclusive. In certain embodiments of the compound of structural Formula (IV), n is between 1 and 10, inclusive. In certain embodiments of the compound of structural Formula (IV), n is between 1 and 5, inclusive. In certain embodiments of the compound of structural Formula (IV), n is chosen from 1 and 2. In certain embodiments of the compound of structural Formula (IV), n is 1.

In certain embodiments, V is —HN—CH₂CH₂CH₂—CO—.

In certain embodiments, the compounds have structural Formula (V):

X—C(═O)—CH₂CH₂—(Y₅—Y₆—Y₇)ₙ—NH—Y₀ Formula (V)

or a salt thereof, wherein:
X comprises a recruiting moiety that is capable of noncovalent binding to a regulatory molecule within the nucleus;
each Y₅—Y₆-Y₇ is independently chosen from β-Py-Im and β-Im-Im;
Y₀ is an end subunit which comprises a moiety chosen from a heterocyclic ring or a straight chain aliphatic segment, which is chemically linked to its single neighbor; and
n is an integer between 1 and 200, inclusive.

In certain embodiments of the compounds of structural Formula (V), at most one of Y₅—Y₆-Y₇ is β-Im-Im.

In certain embodiments of the compounds of structural Formula (V), Y₅—Y₆-Y₇ is β-Py-Im.

In certain embodiments of the compound of structural Formula (V), n is between 1 and 100, inclusive. In certain embodiments of the compound of structural Formula (V), n is between 1 and 50, inclusive. In certain embodiments of the compound of structural Formula (V), n is between 1 and 20, inclusive. In certain embodiments of the compound of structural Formula (V), n is between 1 and 10, inclusive. In certain embodiments of the compound of structural Formula (V), n is between 1 and 5, inclusive. In certain embodiments of the compound of structural Formula (V), n is chosen from 1 and 2. In certain embodiments of the compound of structural Formula (V), n is 1.

In certain embodiments, the compounds have structural Formula (VI):

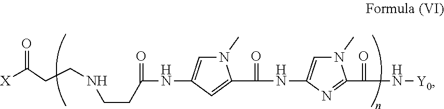

or a salt thereof, wherein:
X comprises a recruiting moiety that is capable of noncovalent binding to a regulatory molecule within the nucleus;
Y₀ is an end subunit which comprises a moiety chosen from a heterocyclic ring or a straight chain aliphatic segment, which is chemically linked to its single neighbor; and
n is an integer between 1 and 200, inclusive.

In certain embodiments of the compound of structural Formula (VI), n is between 1 and 100, inclusive. In certain embodiments of the compound of structural Formula (VI), n is between 1 and 50, inclusive. In certain embodiments of the compound of structural Formula (VI), n is between 1 and 20, inclusive. In certain embodiments of the compound of structural Formula (VI), n is between 1 and 10, inclusive. In certain embodiments of the compound of structural Formula (VI), n is between 1 and 5, inclusive. In certain embodiments of the compound of structural Formula (VI), n is chosen from 1 and 2. In certain embodiments of the compound of structural Formula (VI), n is 1.

In certain embodiments, the compounds have structural Formula (VII):

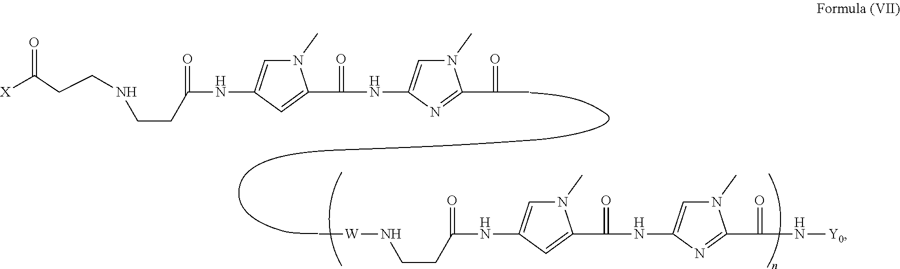

or a salt thereof, wherein:
X comprises a recruiting moiety that is capable of noncovalent binding to a regulatory molecule within the nucleus; and
W is a spacer;
Y₀ is an end subunit which comprises a moiety chosen from a heterocyclic ring or a straight chain aliphatic segment, which is chemically linked to its single neighbor; and
n is an integer between 1 and 200, inclusive.

In certain embodiments of the compound of structural Formula (VII), n is between 1 and 100, inclusive. In certain embodiments of the compound of structural Formula (VII), n is between 1 and 50, inclusive. In certain embodiments of the compound of structural Formula (VII), n is between 1 and 20, inclusive. In certain embodiments of the compound of structural Formula (VII), n is between 1 and 10, inclusive. In certain embodiments of the compound of structural Formula (VII), n is between 1 and 5, inclusive. In certain embodiments of the compound of structural Formula (VII), n is chosen from 1 and 2. In certain embodiments of the compound of structural Formula (VII), n is 1.

In certain embodiments of the compounds of structural Formula (VII), wherein: W is —NHCH₂—(CH₂OCH₂)ₚ—CH₂CO—; and p is an integer between 1 and 4, inclusive.

In some embodiments, (V) is —(CH₂)ₙ—NR¹—(CH₂)_{b}—, —(CH₂)ₙ—, —(CH₂)ₙ—O—(CH₂)_{b}—, —(CH₂)ₙ—CH(NHR¹)—, —(CH₂)ₙ—CH(NHR¹)—, —(CR²R3)ₐ—, or —(CH₂)ₙ—CH(NR¹₃)⁺—(CH₂)_{b}—, wherein each a is independently an integer between 2 and 4; R¹ is H, an optionally substituted C₁₋₆ alkyl, an optionally substituted C₃₋₁₀ cycloalkyl, an optionally substituted C₆₋₁₀ aryl, an optionally substituted 4-10 membered heterocyclyl, or an optionally substituted 5-10 membered heteroaryl; each R² and R³ are independently H, halogen, OH, NHAc, or C₁₋₄ alky. In some embodiments, R¹ is H. In some embodiments, R¹ is C₁₋₆ alkyl optionally substituted by 1-3 substituents selected from —C(O)-phenyl. In some embodiments, (V) is —(CR²R³)—(CH₂)ₙ— or —(CH₂)ₙ—(CR²R³)—(CH₂)_{b}—, wherein each a is independently 1-3, b is 0-3, and each R² and R³ are independently H, halogen, OH, NHAc, or C₁₋₄ alky. In some embodiments, (V) is —(CH₂)—CH(NH₃)⁺—(CH₂)— or —(CH₂)—CH₂CH(NH₃)⁺—.

In one aspect, the compounds of the present disclosure bind to the GAA of fxn and recruit a regulatory moiety to the vicinity of fxn. The regulatory moiety, due to its proximity to the gene, will be more likely to modulate the expression of fxn.

Also provided are embodiments wherein any compound disclosed above, including compounds of Formulas (I)-(VIII), are singly, partially, or fully deuterated. Methods for accomplishing deuterium exchange for hydrogen are known in the art.

Also provided are embodiments wherein any embodiment above may be combined with any one or more of these embodiments, provided the combination is not mutually exclusive.

As used herein, two embodiments are “mutually exclusive” when one is defined to be something which is different than the other. For example, an embodiment wherein two groups combine to form a cycloalkyl is mutually exclusive with an embodiment in which one group is ethyl the other group is hydrogen. Similarly, an embodiment wherein one group is CH₂ is mutually exclusive with an embodiment wherein the same group is NH.

In one aspect, the compounds of the present disclosure bind to the GAA of fxn and recruit a regulatory moiety to the vicinity of fxn. The regulatory moiety, due to its proximity to the gene, will be more likely to modulate the expression of fxn.

In one aspect, the compounds of the present disclosure provide a polyamide sequence for interaction of a single polyamide subunit to each base pair in the GAA repeat sequence. In one aspect, the compounds of the present disclosure provide a turn component V, in order to enable hairpin binding of the compound to the GAA, in which each nucleotide pair interacts with two subunits of the polyamide.

In one aspect, the compounds of the present disclosure provide more than one copy of the polyamide sequence for noncovalent binding to the fxn, and the individual polyamide sequences in this compound are linked by a spacer W, as defined above. The spacer W allows this compound to adjust its geometry as needed to alleviate the geometric strain that otherwise affects the noncovalent binding of longer polyamide sequences.

### First Terminus DNA Binding Moiety

The first terminus interacts and binds with the gene, particularly with the minor grooves of the GAA sequence. In one aspect, the compounds of the present disclosure provide a polyamide sequence for interaction of a single polyamide subunit to each base pair in the GAA repeat sequence. In one aspect, the compounds of the present disclosure provide a turn component (e.g., aliphatic amino acid moiety), in order to enable hairpin binding of the compound to the GAA, in which each nucleotide pair interacts with two subunits of the polyamide.

In one aspect, the compounds of the present disclosure are more likely to bind to the repeated GAA of fxn than to GAA elsewhere in the subject's DNA, due to the high number of GAA repeats associated with fxn.

In one aspect, the compounds of the present disclosure provide more than one copy of the polyamide sequence for noncovalent binding to GAA. In one aspect, the compounds of the present disclosure bind to fxn with an affinity that is greater than a corresponding compound that contains a single polyamide sequence.

In one aspect, the compounds of the present disclosure provide more than one copy of the polyamide sequence for noncovalent binding to the GAA, and the individual polyamide sequences in this compound are linked by a spacer W, as defined above. The spacer W allows this compound to adjust its geometry as needed to alleviate the geometric strain that otherwise affects the noncovalent binding of longer polyamide sequences.

In certain embodiments, the DNA recognition or binding moiety binds in the minor groove of DNA.

In certain embodiments, the DNA recognition or binding moiety comprises a polymeric sequence of monomers, wherein each monomer in the polymer selectively binds to a certain DNA base pair.

In certain embodiments, the DNA recognition or binding moiety comprises a polyamide moiety.

In certain embodiments, the DNA recognition or binding moiety comprises a polyamide moiety comprising heteroaromatic monomers, wherein each heteroaromatic monomer binds noncovalently to a specific nucleotide, and each heteroaromatic monomer is attached to its neighbor or neighbors via amide bonds.

In certain embodiments, the DNA recognition moiety binds to a sequence comprising at least 1000 pentanucleotide repeats. In certain embodiments, the DNA recognition moiety binds to a sequence comprising at least 500 trinucleotide repeats. In certain embodiments, the DNA recognition moiety binds to a sequence comprising at least 200 trinucleotide repeats. In certain embodiments, the DNA recognition moiety binds to a sequence comprising at least 100 trinucleotide repeats. In certain embodiments, the DNA recognition moiety binds to a sequence comprising at least 50 trinucleotide repeats. In certain embodiments, the DNA recognition moiety binds to a sequence comprising at least 20 trinucleotide repeats.

In certain embodiments, the compounds comprise a cell-penetrating ligand moiety.

In certain embodiments, the cell-penetrating ligand moiety is a polypeptide.

In certain embodiments, the cell-penetrating ligand moiety is a polypeptide containing fewer than 30 amino acid residues.

In certain embodiments, the polypeptide is chosen from any one of SEQ ID NO. 1 to SEQ ID NO. 37, inclusive.

The form of the polyamide selected can vary based on the target gene. The first terminus can include a polyamide selected from the group consisting of a linear polyamide, a hairpin polyamide, a H-pin polyamide, an overlapped polyamide, a slipped polyamide, a cyclic polyamide, a tandem polyamide, and an extended polyamide. In some embodiments, the first terminus comprises a linear polyamide. In some embodiments, the first terminus comprises a hairpin polyamide.

The binding affinity between the polyamide and the target gene can be adjusted based on the composition of the polyamide. In some embodiments, the polyamide is capable of binding the DNA with an affinity of less than about 600 nM, about 500 nM, about 400 nM, about 300 nM, about 250 nM, about 200 nM, about 150 nM, about 100 nM, or about 50 nM. In some embodiments, the polyamide is capable of binding the DNA with an affinity of less than about 300 nM. In some embodiments, the polyamide is capable of binding the DNA with an affinity of less than about 200 nM. In some embodiments, the polyamide is capable of binding the DNA with an affinity of greater than about 200 nM, about 150 nM, about 100 nM, about 50 nM, about 10 nM, or about 1 nM. In some embodiments, the polyamide is capable of binding the DNA with an affinity in the range of about 1-600 nM, 10-500 nM, 20-500 nM, 50-400 nM, or 100-300 nM.

The binding affinity between the polyamide and the target DNA can be determined using a quantitative footprint titration experiment. The experiment involve measuring the dissociation constant K_{d} of the polyamide for target sequence at either 24° C. or 37° C., and using either standard polyamide assay solution conditions or approximate intracellular solution conditions.

The binding affinity between the regulatory protein and the ligand on the second terminus can be determined using an assay suitable for the specific protein. The experiment involve measuring the dissociation constant K_{d} of the ligand for protein and using either standard protein assay solution conditions or approximate intracellular solution conditions.

In some embodiments, the first terminus comprises —NH-Q-C(O)—, wherein Q is an optionally substituted C₆₋₁₀ arylene group, optionally substituted 4-10 membered heterocyclene, optionally substituted 5-10 membered heteroarylene group, or an optionally substituted alkylene group. In some embodiments, Q is an optionally substituted C₆₋₁₀ arylene group or optionally substituted 5-10 membered heteroarylene group. In some embodiments, Q is an optionally substituted 5-10 membered heteroarylene group. In some embodiments, the 5-10 membered heteroarylene group is optionally substituted with 1-4 substituents selected from H, OH, halogen, C₁₋₁₀ alkyl, NO₂, CN, NR′R″, C₁₋₆ haloalkyl, C₁₋₆ alkoxyl, C₁₋₆ haloalkoxy, (C₁₋₆ alkoxy)C₁₋₆ alkyl, C₂-10 alkenyl, C₂-10 alkynyl, C₃₋₇ carbocyclyl, 4-10 membered heterocyclyl, C₆₋₁₀ aryl, 5-10 membered heteroaryl, (C₃₋₇carbocyclyl)C₁₋₆ alkyl, (4-10 membered heterocyclyl)C₁₋₆ alkyl, (C₆₋₁₀ aryl)C₁₋₆ alkyl, (C₆₋₁₀ aryl)C₁₋₆ alkoxy, (5-10 membered heteroaryl)C₁₋₆ alkyl, (C₃.carbocyclyl)-amine, (4-10 membered heterocyclyl)amine, (C₆₋₁₀ aryl)amine, (5-10 membered heteroaryl)amine, acyl, C-carboxy, O-carboxy, C-amido, N-amido, S-sulfonamido, N-sulfonamido, —SR′, COOH, or CONR′R″; wherein each R′ and R″ are independently H, C₁₋₁₀ alkyl, C₁₋₁₀ haloalkyl, C₁₋₁₀ alkoxyl.

In some embodiments, the first terminus comprises at least three aromatic carboxamide moieties selected to correspond to the nucleotide repeat sequence GAA and at least one aliphatic amino acid residue chosen from the group consisting of glycine, β-alanine, γ-aminobutyric acid, 2,4-diaminobutyric acid, and 5-aminovaleric acid. In some embodiments, the first terminus comprises at least one β-alanine subunit.

In some embodiments, the monomer element is independently selected from the group consisting of optionally substituted pyrrole carboxamide monomer, optionally substituted imidazole carboxamide monomer, optionally substituted C—C linked heteromonocyclic/heterobicyclic moiety, and β-alanine.

In some embodiments, the first terminus comprises a structure of Formula (A-1), or a pharmaceutically acceptable salt thereof:

-L₁ₐ-[A-M]ₚ-E₁ (A-1)

wherein,
each [A-M] appears p times and p is an integer in the range of 1 to 10,
L₁ₐ is a bond, a C₁₋₆ alkylene, —NR^{a}—C₁₋₆ alkylene-C(O)—, —NR^{a}C(O)—, —NR^{a}—C₁₋₆ alkylene, —O—, or —O—C₁₋₆ alkylene;
each A is selected from the group consisting of a bond, C₁₋₁₀ alkylene, optionally substituted C₆₋₁₀ arylene group, optionally substituted 4-10 membered heterocyclene, optionally substituted 5-10 membered heteroarylene group, —C₁₋₁₀ alkylene-C(O)—, —C₁₋₁₀ alkylene-NR^{a}—, —CO—, —NR^{a}—, —CONR^{a}—, —CONR^{a}C₁₋₄alkylene-, —NR^{a}CO—C₁₋₄alkylene-, —C(O)O—, —O—, —S—, —S(O)—, —S(O)₂—, —C(═S)—NH—, —C(O)—NH—NH—, —C(O)—N═N—, —C(O)—CH═CH—, —(CH₂)₀₋₄—CH═CH—(CH₂)₀₋₄—, —N(CH₃)—C₁₋₆ alkylene-, and

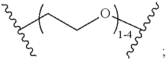

—NH— C₁₋₆ alkylene-NH—, —O— C₁₋₆ alkylene-O—, —NH—N═N—, —NH—C(O)—NH—, and any combinations thereof, and at least one A is —CONH—;
each M is an optionally substituted C₆₋₁₀ arylene group, optionally substituted 4-10 membered heterocyclene, optionally substituted 5-10 membered heteroarylene group, or an optionally substituted
alkylene;
E₁ is H or -A^{E}_G
A^{E} is absent or —NHCO—;
G is selected from the group consisting of optionally substituted C₆₋₁₀ aryl, optionally substituted 4-10 membered heterocyclyl, optionally substituted 5-10 membered heteroaryl, an optionally substituted C₁₋₆ alkyl, C₀₋₄alkylene-NHC(═NH)NH, —CN, —C₀₋₄alkylene-C(═NH)(NR^{a}R^{b}), —C₀₋₄alkylene-C(═N⁺H₂)(NR^{a}R^{b})C₁₋₅alkylene-NR^{a}R^{b}, C₀₋₄ alkylene-NHC(═NH)R^{a}, and optionally substituted amine; and
each R^{a} and R^{b} are independently selected from the group consisting of H, an optionally substituted C₁₋₆ alkyl, an optionally substituted C₃₋₁₀ cycloalkyl, optionally substituted C₆₋₁₀ aryl, optionally substituted 4-10 membered heterocyclyl, and optionally substituted 5-10 membered heteroaryl.

In some embodiments, the first terminus comprises a polyamide having the structure of Formula (A-2), or a pharmaceutically acceptable salt thereof:

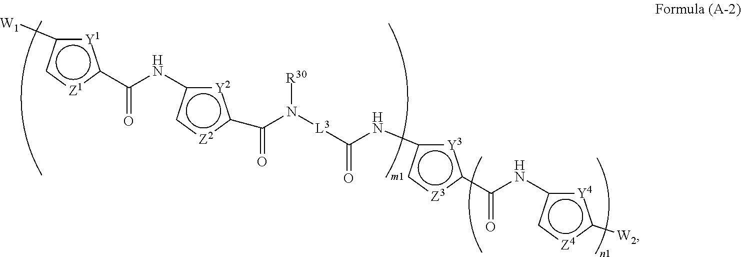

wherein;
m₁ is 1-4;
n₁ is 0-2;
each Y¹, Y², Y³, and Y⁴ is independently CH or N;
each Z¹, Z², Z³, and Z⁴ is independently O, S, or NR^{1D};
each L³ is an optionally substituted C₁-C₆ alkylene, C₃-C₇ cycloalkylene, 3 to 7-membered heterocyclene, or 5 to 6-membered heteroarylene;
each R³⁰ is hydrogen or an C₁-C₆ alkyl; or
each R³⁰ and L³ join together with the atom(s) to which they are attached to form a 4-to 7-membered heterocyclic ring;
W₁ is hydrogen, an optionally substituted C₁-C₆ alkyl, —NR¹E-C(O)—NR^{1E}R^{1F}, —C(O)—NR^{1E}R^{F} or (AA)₁₋₁₀;
W₂ is hydrogen, optionally substituted C₁-C₆ alkyl, —C(O)—NR^{1E}R^{1F}, or (AA)₁₋₁₀;
each R^{1D} and R^{1E} is independently hydrogen, optionally substituted C₁-C₅₀ alkyl, C₁-C₅₀ heteroalkyl, or PEG₁₋₅₀;
R^{1F} is hydrogen, optionally substituted C₁-C₂₀ alkyl, C₁-C₂₀ heteroalkyl, PEG₁₋₂₀, or one or more AA; and
each AA is independently a naturally occurring amino acid.

In some embodiments, each L³ is an optionally substituted C₁-C₆ alkylene. In some embodiments, L³ is a C₂, C₃, C₄, or C₅ alkylene optionally substituted with one or more hydrogen, halogen, C₁-C₆ alkyl, C₁-C₆ heteroalkyl, C₁-C₆ haloalkyl, C₁-C₆ hydroxyalkyl, C₃-C₆ cycloalkyl or 4 to 7-membered heterocycloalkyl ring. In some embodiments, L³ is a C₂ or C₃ alkylene optionally substituted with one or more hydrogen, halogen, C₁-C₆ alkyl, C₁-C₆ heteroalkyl, C₃-C₆ cycloalkyl or 4 to 7-membered heterocycloalkyl ring. In some embodiments, L³ is a C₂ alkylene optionally substituted with one or two hydrogen, C₁-C₆ alkyl, C₁-C₆ heteroalkyl, C₃-C₆ cycloalkyl or 4 to 7-membered heterocycloalkyl ring.

In some embodiments, each L³ is independently C₃-C₇ cycloalkylene. In some embodiments, L³ is a cyclobutylene, cyclopentylene, cyclohexylene, or cycloheptylene ring. In some embodiments, L³ is cyclobutylene. In some embodiments, L³ is cyclopentylene. In some embodiments, L³ is cyclohexylene.

In some embodiments, each L³ is 3 to 7-membered heterocyclene. In some embodiments, L³ is a 4-membered, 5-membered, or 6-membered heterocyclene.

In some embodiments, each R³⁰ is independently hydrogen. In some embodiments, each R³⁰ is independently C₁-C₆ alkyl.

In some embodiments, L³ and R³⁰ join together with the atoms to which they are attached to form a 4- to 7-membered heterocyclic ring. In some embodiments, the ring is a 4-membered heterocyclic ring. In some embodiments, the ring is a 5-membered heterocyclic ring. In some embodiments, the ring is a 6-membebered heterocyclic ring. In some embodiments, the ring is a 7-membered heteroaromatic ring.

In some embodiments, the first terminus comprises a polyamide having the structure of Formula (A-3), or a pharmaceutically acceptable salt thereof:

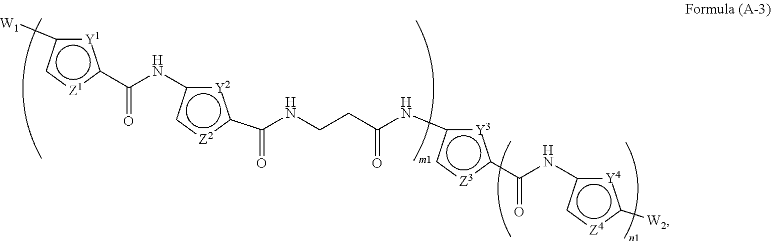

wherein,
m₁ is 1-4;
n₁ is 0-2;
each Y¹, Y², Y³, and Y⁴ is independently CH or N;
each Z¹, Z², Z³, and Z⁴ is independently O, S, or NR^{1D};
W₁ is hydrogen, optionally substituted C₁-C₆ alkyl, —NR¹E-C(O)—NR^{1E}R^{1F}, —C(O)—NR^{1E}R^{1F}, or (AA)₁₋₁₀;
W₂ is hydrogen, optionally substituted C₁-C₆ alkyl, —C(O)—NR^{1E}R^{1F}, or (AA)₁₋₁₀;
each R^{1D} and R^{1E} is independently hydrogen, optionally substituted C₁-C₅₀ alkyl, C₁-C₅₀ heteroalkyl, or PEG₁₋₅₀;
R^{1F} is hydrogen, optionally substituted C₁-C₂₀ alkyl, C₁-C₂₀ heteroalkyl, PEG₁₋₂₀, or one or more AA; and
each AA is independently an amino acid residue selected from β-alanine, lysine, and arginine.

In some embodiments, the linker moiety is connected to the DNA binding moiety (i.e. polyamide) at W₂. In some embodiments, W₂ is an optionally substituted C₁-C₆ alkyl, —C(O)—NR^{1E}R^{1F}, or (AA)₁₋₁₀. In some embodiments, W² is hydrogen.

In some embodiments, W² is (AA)₁₋₁₀. In some embodiments the AA is β-alanine. In some embodiments the AA is one β-alanine. In some embodiments, AA is two β-alanines.

In some embodiments, the first terminus comprises a polyamide having the structure of Formula (A-4), or a pharmaceutically acceptable salt thereof:

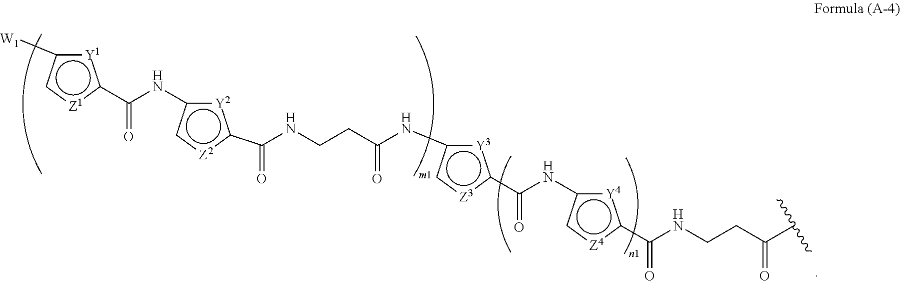

In some embodiments, each R^{1D} and R^{1E} is independently hydrogen, optionally substituted C₁-C₂₀ alkyl, C₁-C₂₀ heteroalkyl, or PEG₁₋₂₀. In some embodiments, each R^{1D} and R^{1E} is independently hydrogen, optionally substituted C₁-C₁₀ alkyl, C₁-C₁₀ heteroalkyl, or PEG₁₋₂₀.

In some embodiments, each R″ is independently optionally substituted C₁-C₂₀ alkyl, C₁-C₂₀ heteroalkyl, or PEG₁₋₂₀, each of which is optionally substituted with amido, alkyl, alkynyl, azido, amino, halogen, haloalkyl, hydroxy, nitro, oxo (═O), phosphorous hydroxide, or PEG. In some embodiments, each R^{1D} is independently optionally substituted C₁-C₂₀, optionally substituted with —CN, —NH₂, —N₃, —OH, CF₃, —OP(═O)(OH)₂, —OP(═O)(OCH₃)₂, —OP(═O)(OCH₃)(OH), or —OP(═O)₂OH. In some embodiments, each R^{1D} is independently PEG₁₋₅₀. In some embodiments, each R^{1D} is independently —C(O)—NR²R^{2B} or —NR²R²¹, wherein each R^{2A} and R²¹ is independently hydrogen, C₁-C₅₀ alkyl, or PEG₁₋₅₀.

In some embodiments, each Z¹, Z², Z³, and Z⁴ is independently O or S.

In some embodiments, each Z¹, Z², Z³, and Z⁴ is independently NR¹D, wherein R^{1D} is optionally substituted C₁-C₂₀ alkyl or C₁-C₂₀ heteroalkyl.

In some embodiments, each Z¹, Z², Z³, and Z⁴ is independently NCH₃.

In some embodiments, each Z¹, Z², Z³, and Z⁴ is independently NH.

In some embodiments, the first terminus comprises a polyamide having the structure of Formula (A-5), or a pharmaceutically acceptable salt thereof:

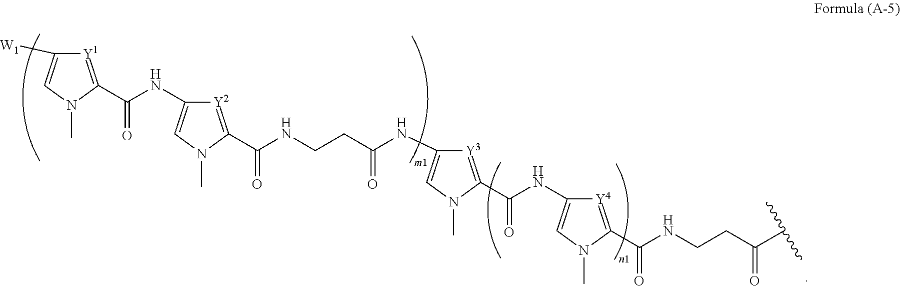

In some embodiments, each Y¹ and Y³ are N; and each Y² and Y⁴ are independently CH or N. In some embodiments, each Y² and Y⁴ is independently CH. In some embodiments, each Y² and Y⁴ is independently N. In some embodiments, Y² is CH and Y⁴ is N. In some embodiments, Y² is N and Y⁴ is CH.

In some embodiments, each unit m₁ and n₁ are different or the same. In some embodiments, each unit m₁ is different. In some embodiments, each unit m₁ is the same. In some embodiments, each unit n₁ is different. In some embodiments, each unit n₁ is the same.

In some embodiments, m₁ is 2 or 3; and n₁ is 0 or 1.

In some embodiments, m₁ is 2. In some embodiments, m₁ is 1.

In some embodiments, n₁ is 0. In some embodiments, n₁ is 1.

In some embodiments, the linker moiety is connected to the DNA binding moiety through W₁. In some embodiments, W₁ is optionally substituted C₁-C₆ alkyl, or —C(O)—NR^{1E}R^{1F}. In some embodiments, W₁ is —C(O)—NR^{1E}R^{1F}, wherein R^{1E} is hydrogen; and R^{1F} is hydrogen, optionally substituted C₁-C₁₀ alkyl, or PEG₁₋₂₀.

In some embodiments, W₁ is hydrogen.

In some embodiments, the first terminus comprises a polyamide having the structure of Formula (A-6), or a pharmaceutically acceptable salt thereof:

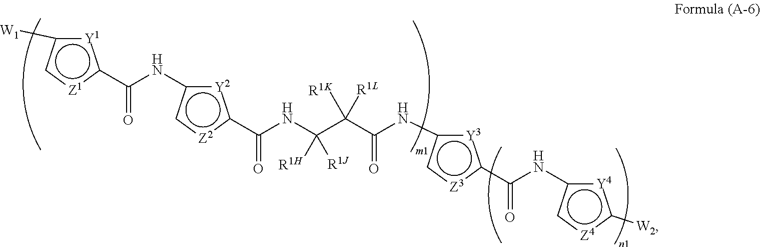

wherein,
each R^{1H}, R^{1J}, R^{1K}, and R^{1L} is independently hydrogen, halogen, C₁-C₆ alkyl, C₁-C₆ heteroalkyl, C₁-C₆ haloalkyl, or C₁-C₆ hydroxyalkyl; or
R^{1H} and R^{1J} or R^{1L} and R^{1K} combine together with the atom to which they are attached to form a C₃-C₆ cycloalkyl or 4 to 7-membered heterocycloalkyl ring.

In some embodiments, each R^{1H}, R^{1J}, R^{1K}, and R^{IL} is independently hydrogen, halogen, C₁-C₆ alkyl, C₁-C₆ heteroalkyl, C₁-C₆ haloalkyl, or C₁-C₆ hydroxyalkyl. In some embodiments, each R^{1H}, R^{1J}, R^{1K}, and R^{1L} is independently hydrogen, halogen, or C₁-C₆ alkyl. In some embodiments, each R^{1H}, R^{1J}, R^{1K}, and R^{1L} is independently halogen. In some embodiments, each R^{1H}, R^{J}, R^{1K}, and R^{IL} is independently C₁-C₆ alkyl. In some embodiments, each R^{1H}, R^{1J}, R^{1K}, and R^{1L} is independently hydrogen.

In some embodiments, R^{1H} and R^{1J} or R^{1L} and R^{1K} combine together with the atom to which they are attached to form a C₃-C₆ cycloalkyl or 4 to 7-membered heterocycloalkyl ring. In some embodiments, R^{1H} and R^{1J} or R^{IL} and R^{1K} combine together with the atom to which they are attached to form a C₃-C₆ cycloalkyl. In some embodiments, R^{1H} and R^{u} or R^{1L} and R^{1K} combine together with the atom to which they are attached to form a 4 to 7-membered heterocycloalkyl ring.

In some embodiments, the first terminus comprises a polyamide having the structure of Formula (A-7), or a pharmaceutically acceptable salt thereof:

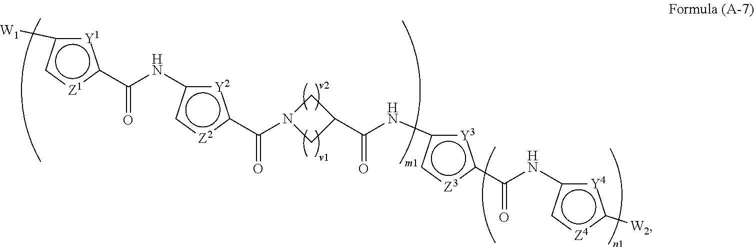

wherein each v₁ and v₂ are independently 1-3.

In some embodiments, each v₁ is independently 1. In some embodiments, each v₁ is independently 2. In some embodiments, each v₁ is independent 3. In some embodiments, each v₂ is independently 1. In some embodiments, each v₂ is independently 2. In some embodiments, each v₂ is independent 3.

In some embodiments, the first terminus comprises a polyamide having the structure of Formula (A-8), or a pharmaceutically acceptable salt thereof:

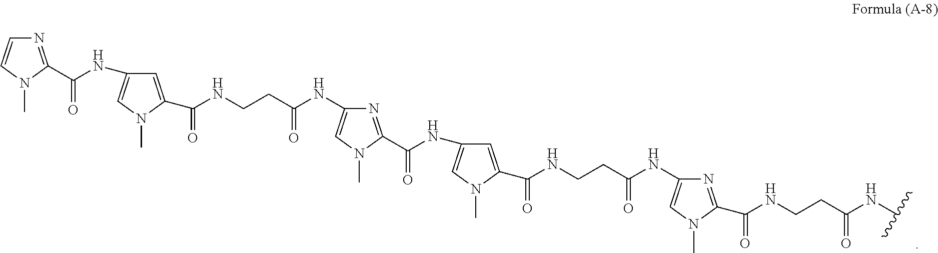 [SMILES: *NC(=O)CCNC(=O)c1nc(NC(=O)CCNC(=O)c2cc(NC(=O)c3nc(NC(=O)CCNC(=O)c4cc(NC(=O)c5nccn5C)cn4C)cn3C)cn2C)cn1C]

In some embodiments, the first terminus comprises a polyamide having the structure of Formula (A-9) or a pharmaceutically acceptable

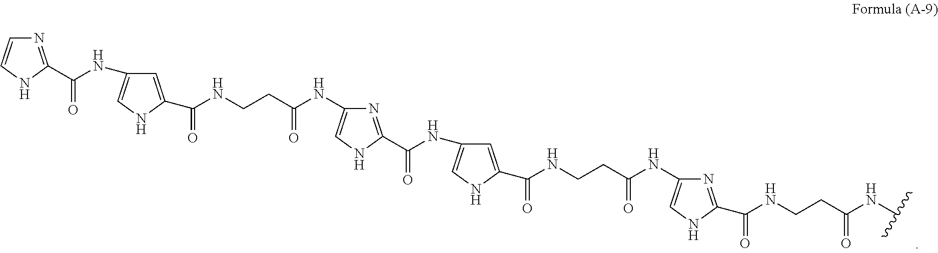 [SMILES: *NC(=O)CCNC(=O)c1nc(NC(=O)CCNC(=O)c2cc(NC(=O)c3nc(NC(=O)CCNC(=O)c4cc(NC(=O)c5ncc[nH]5)c[nH]4)c[nH]3)c[nH]2)c[nH]1]

In some embodiments, the first terminus comprises a polyamide having the structure of Formula (A-10), or a pharmaceutically acceptable salt thereof:

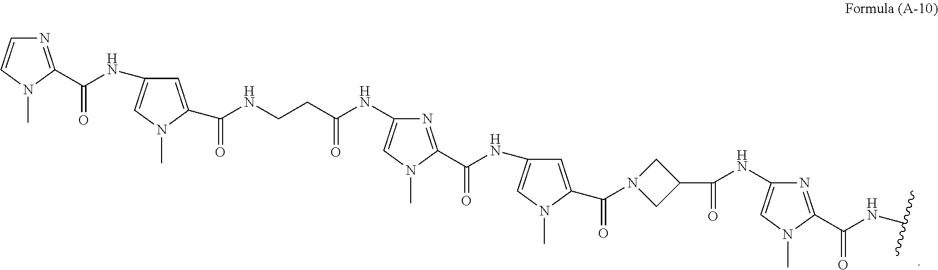 [SMILES: *NC(=O)c1nc(NC(=O)C2CN(C(=O)c3cc(NC(=O)c4nc(NC(=O)CCNC(=O)c5cc(NC(=O)c6nccn6C)cn5C)cn4C)cn3C)C2)cn1C]

In some embodiments, the first terminus comprises a polyamide having the structure of Formula (A-11), or a pharmaceutically acceptable salt thereof:

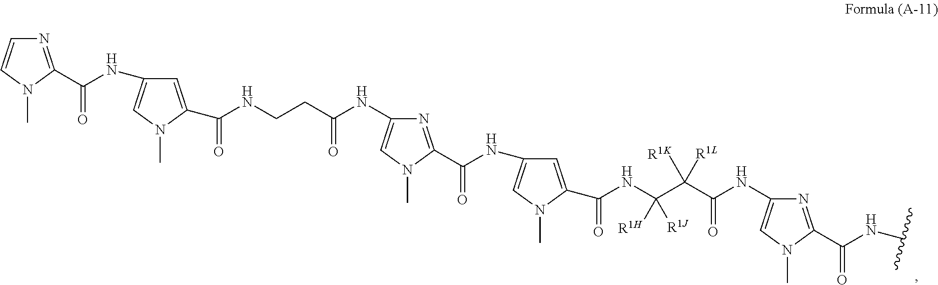

wherein,
each R^{1H}, R^{1J}, R^{1K}, and R^{1L} is independently hydrogen, halogen, C₁-C₆ alkyl, C₁-C₆ heteroalkyl, C₁-C₆ haloalkyl, or C₁-C₆ hydroxyalkyl; or
R^{1H} and R^{1J} or R^{1L} and R^{1K} combine together with the atom to which they are attached to form a C₃-C₆ cycloalkyl or 4 to 7-membered heterocycloalkyl ring R^{1H} and R¹¹ or R^{IL} and R^{1K} combine together with the atom to which they are attached to form a C₃-C₆ cycloalkyl or 4 to 7-membered heterocycloalkyl ring.

In some embodiments, the first terminus comprises a polyamide having the structure of Formula (A-12), or a pharmaceutically acceptable salt thereof:

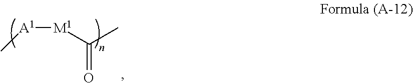

wherein;
each A¹ is —NH— or —NH—(CH₂)ₘ—CH₂—C(O)—NH—;
each M is an optionally substituted C₆₋₁₀ arylene group, optionally substituted 4-10 membered heterocyclene, optionally substituted 5-10 membered heteroarylene group, or optionally substituted alkylene;
m is an integer between 1 to 10; and
n is an integer between 1 and 6.

In some embodiments, each M¹ in [A¹-M¹] of Formula (A-6) is a C₆₋₁₀ arylene group, 4-10 membered heterocyclene, optionally substituted 5-10 membered heteroarylene group, or C₁₋₆ alkylene; each optionally substituted by 1-3 substituents selected from H, OH, halogen, C₁₋₁₀ alkyl, NO₂, CN, NR′R″, C₁₋₆ haloalkyl, —C₁₋₆ alkoxyl, C₁₋₆ haloalkoxy, (C₁₋₆ alkoxy)C₁₋₆ alkyl, C₂₋₁₀ alkenyl, C₂₋₁₀ alkynyl, C₃₋₇ carbocyclyl, 4-10 membered heterocyclyl 4-10 membered heterocyclyl, C₆₋₁₀aryl, 5-10 membered heteroaryl, —(C₃. 7carbocyclyl)C₁₋₆alkyl, (4-10 membered heterocyclyl)C₁₋₆alkyl, (C₆₋₁₀aryl)C₁₋₆alkyl, (C₆₋₁₀aryl)C₁₋₆alkoxy, (5-10 membered heteroaryl)C₁₋₆alkyl, —(C₃₋₇carbocyclyl)-amine, (4-10 membered heterocyclyl)amine, (C₆₋₁₀ aryl)amine, (5-10 membered heteroaryl)amine, acyl, C-carboxy, O-carboxy, C-amido, N-amido, S-sulfonamido, N-sulfonamido, —SR′, COOH, or CONR′R″; wherein each R′ and R″ are independently H, C₁₋₁₀ alkyl, C₁₋₁₀ haloalkyl, —C₁₋₁₀ alkoxyl. In some embodiments, each R¹ in [A¹-R¹] of Formula (A-12) is a 5-10 membered heteroarylene containing at least one heteroatoms selected from O, S, and N or a C₁₋₆ alkylene, and the heteroarylene or the a C₁₋₆ alkylene is optionally substituted with 1-3 substituents selected from OH, halogen, C₁₋₁₀ alkyl, NO₂, CN, NR′R″, C₁₋₆ haloalkyl, —C₁₋₆ alkoxyl, C₁₋₆ haloalkoxy, C₃₋₇ carbocyclyl, 4-10 membered heterocyclyl, C₆₋₁₀aryl, 5-10 membered heteroaryl, —SR′, COOH, or CONR′R″; wherein each R′ and R″ are independently H, C₁₋₁₀ alkyl, C₁₋₁₀ haloalkyl, —C₁₋₁₀ alkoxyl. In some embodiments, each R¹ in [A¹-R¹] of Formula (A-12) is a 5-10 membered heteroarylene containing at least one heteroatoms selected from O, S, and N, and the heteroarylene is optionally substituted with 1-3 substituents selected from OH, C₁₋₆ alkyl, halogen, and C₁₋₆ alkoxyl.

The DNA recognition, binding moiety, or polyamide can include one or more subunits selected from the group consisting of:

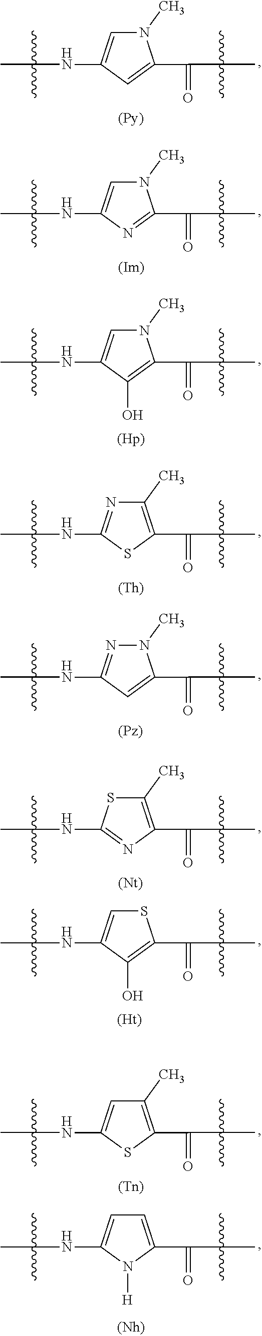

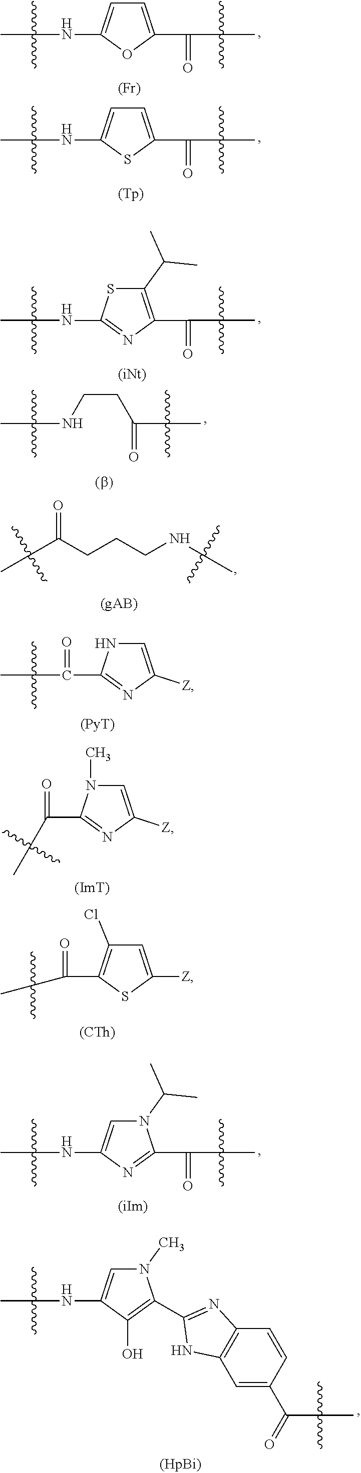

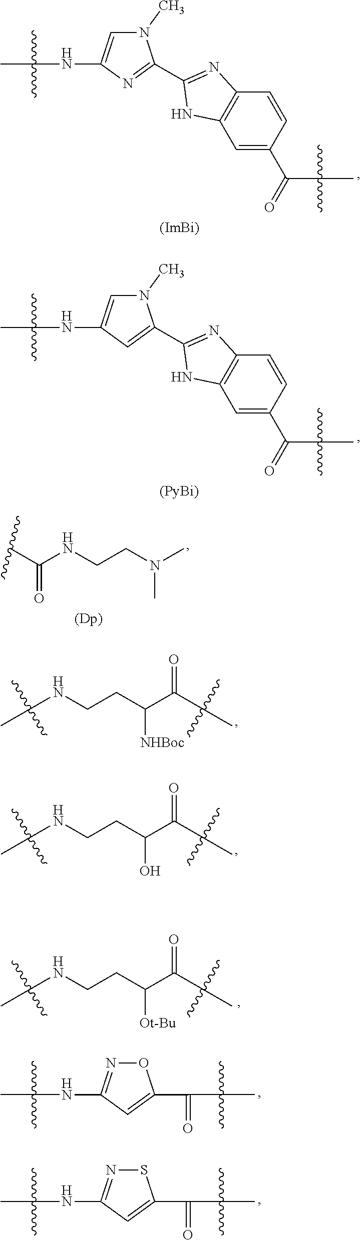

—NH-benzopyrazinylene-CO—, —NH-phenylene-CO—, —NH-pyridinylene-CO—, —NH-piperidinylene-CO—, —NH-pyrimidinylene-CO—, —NH-anthracenylene-CO—, —NH-quinolinylene-CO—, and

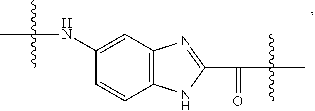

wherein Z is H, NH₂, C₁₋₆ alkyl, or C₁₋₆ alkylNH₂.

In some embodiments, Py is

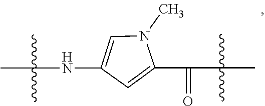 [SMILES: *Nc1cc(C(*)=O)n(C)c1]

### Im is

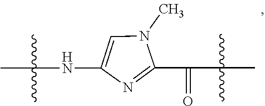 [SMILES: *Nc1cn(C)c(C(*)=O)n1]

### Hp is

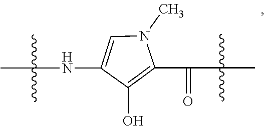 [SMILES: *Nc1cn(C)c(C(*)=O)c1O]

### Th is

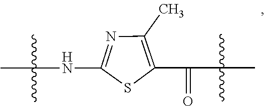 [SMILES: *Nc1nc(C)c(C(*)=O)s1]

### Pz is

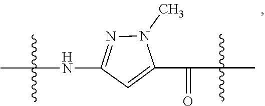 [SMILES: *Nc1cc(C(*)=O)n(C)n1]

### Nt is

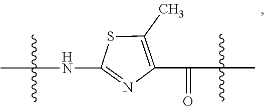 [SMILES: *Nc1nc(C(*)=O)c(C)s1]

### Tn

is

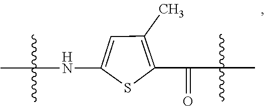 [SMILES: *Nc1cc(C)c(C(*)=O)s1]

### Nh is

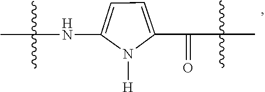 [SMILES: *Nc1ccc(C(*)=O)[nH]1]

iNt is

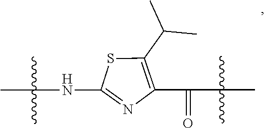 [SMILES: *Nc1nc(C(*)=O)c(C(C)C)s1]

iIm is

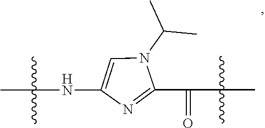 [SMILES: *Nc1cn(C(C)C)c(C(*)=O)n1]

### HpBi is

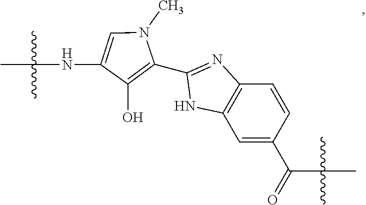 [SMILES: *Nc1cn(C)c(-c2nc3ccc(C(*)=O)cc3[nH]2)c1O]

### ImBi is

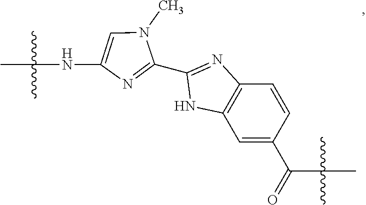 [SMILES: *Nc1cn(C)c(-c2nc3ccc(C(*)=O)cc3[nH]2)n1]

### PyBi is

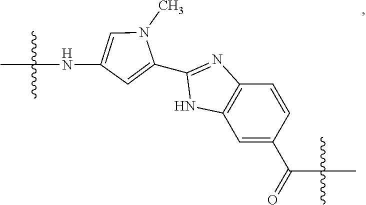 [SMILES: *Nc1cc(-c2nc3ccc(C(*)=O)cc3[nH]2)n(C)c1]

### Dp is

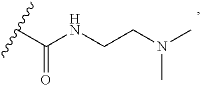 [SMILES: *C(=O)NCCN(C)C]

### —NH-pyridinylene-CO— is

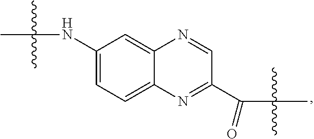 [SMILES: *Nc1ccc2nc(C(*)=O)cnc2c1]

### —NH-piperidinylene-CO— is

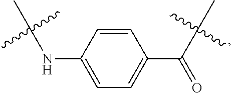 [SMILES: *Nc1ccc(C(*)=O)cc1]

### —NH-pyridinylene-CO— is

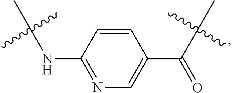 [SMILES: *Nc1ccc(C(*)=O)cn1]

### —NH-piperidinylene-CO— is

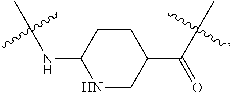 [SMILES: *NC1CCC(C(*)=O)CN1]

### —NH-pyrazinylene-CO— is

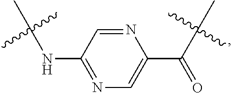 [SMILES: *Nc1cnc(C(*)=O)cn1]

### —NH-anthracenylene-CO— is

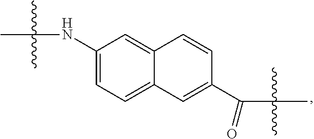 [SMILES: *Nc1ccc2cc(C(*)=O)ccc2c1]

### and —NH-quinolinylene-CO— is

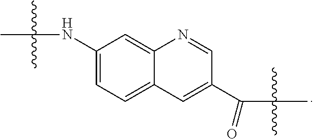 [SMILES: *Nc1ccc2cc(C(*)=O)cnc2c1]

In some embodiments, the first terminus comprises one or more subunits selected from the group consisting of optionally substituted N-methylpyrrole, optionally substituted N-methylimidazole, and β-alanine.

The first terminus in the molecules described herein has a high binding affinity to a sequence having multiple repeats of GAA and binds to the target nucleotide repeats preferentially over other nucleotide repeats or nucleotide sequences. In some embodiments, the first terminus has a higher binding affinity to a sequence having multiple repeats of GAA than to a sequence having repeats of CGG. In some embodiments, the first terminus has a higher binding affinity to a sequence having multiple repeats of GAA than to a sequence having repeats of CCG. In some embodiments, the first terminus has a higher binding affinity to a sequence having multiple repeats of GAA than to a sequence having repeats of CCTG. In some embodiments, the first terminus has a higher binding affinity to a sequence having multiple repeats of GAA than to a sequence having repeats of TGGAA. In some embodiments, the first terminus has a higher binding affinity to a sequence having multiple repeats of GAA than to a sequence having repeats of GGGGCC. In some embodiments, the first terminus has a higher binding affinity to a sequence having multiple repeats of GAA than to a sequence having repeats of CAG. In some embodiments, the first terminus has a higher binding affinity to a sequence having multiple repeats of GAA than to a sequence having repeats of CTG.

Due to the preferential binding between the first terminus and the target nucleotide repeat, the transcription modulation molecules described herein become localized around regions having multiple repeats of GAA. In some embodiments, the local concentration of the first terminus or the molecules described herein is higher near a sequence having multiple repeats of GAA than near a sequence having repeats of CGG. In some embodiments, the local concentration of the first terminus or the molecules described herein is higher near a sequence having multiple repeats of GAA than near a sequence having repeats of CCG. In some embodiments, the local concentration of the first terminus or the molecules described herein is higher near a sequence having multiple repeats of GAA than near a sequence having repeats of CCTG. In some embodiments, the local concentration of the first terminus or the molecules described herein is higher near a sequence having multiple repeats of GAA than near a sequence having repeats of TGGAA. In some embodiments, the local concentration of the first terminus or the molecules described herein is higher near a sequence having multiple repeats of GAA than near a sequence having repeats of GGGGCC. In some embodiments, the local concentration of the first terminus or the molecules described herein is higher near a sequence having multiple repeats of GAA than near a sequence having repeats of CTG. In some embodiments, the local concentration of the first terminus or the molecules described herein is higher near a sequence having multiple repeats of GAA than near a sequence having repeats of CAG.

The first terminus is localized to a sequence having multiple repeats of GAA and binds to the target nucleotide repeats preferentially over other nucleotide repeats. In some embodiments, the sequence has at least 2, 3, 4, 5, 8, 10, 12, 15, 20, 25, 30, 40, 50, 100, 200, 300, 400, or 500 repeats of GAA. In certain embodiments, the sequence comprises at least 1000 nucleotide repeats of GAA. In certain embodiments, the sequence comprises at least 500 nucleotide repeats of GAA. In certain embodiments, the sequence comprises at least 200 nucleotide repeats of GAA. In certain embodiments, the sequence comprises at least 100 nucleotide repeats of GAA. In certain embodiments, the sequence comprises at least 50 nucleotide repeats of GAA. In certain embodiments, the sequence comprises at least 20 nucleotide repeats of GAA.

In one aspect, the compounds of the present disclosure can bind to the repeated GAA of fxn than to GAA elsewhere in the subject's DNA

The polyamide composed of a pre-selected combination of subunits can selectively bind to the DNA in the minor groove. In their hairpin structure, antiparallel side-by-side pairings of two aromatic amino acids bind to DNA sequences, with a polyamide ring packed specifically against each DNA base. N-Methylpyrrole (Py) favors T, A, and C bases, excluding G; N-methylimidazole (Im) is a G-reader; and 3-hydroxyl-N-methylpyrrol (Hp) is specific for thymine base. The nucleotide base pairs can be recognized using different pairings of the amino acid subunits using the paring principle shown in Table 1A and 1B below. For example, an Im/Py pairing reads G·C by symmetry, a Py/Im pairing reads C·G, an Hp/Py pairing can distinguish T·A from A·T, G·C, and C·G, and a Py/Py pairing nonspecifically discriminates both A T and T·A from G·C and C·G.

In some embodiments, the first terminus comprises Im corresponding to the nucleotide G; Py or beta corresponding to the nucleotide A; Py corresponding to the nucleotide A, wherein Im is N-alkyl imidazole, Py is N-alkyl pyrrole, and beta is β-alanine. In some embodiments, the first terminus comprises Im/Py to correspond to the nucleotide pair G/C, Py/beta or Py/Py to correspond to the nucleotide pair A/T, and wherein Im is N-alkyl imidazole (e.g., N-methyl imidazole), Py is N-alkyl pyrrole (e.g., N-methyl pyrrole), and beta is β-alanine.

TABLE 1A
Base paring for single amino acid subunit (Favored (+), disfavored (−).

| Subunit | G | C | A | T |
|---|---|---|---|---|
| Py | − | + | + | + |
| Im | + | − | − | |
| 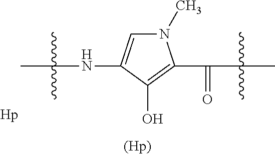 [SMILES: *Nc1cn(C)c(C(*)=O)c1O] | − | − | − | + |
| 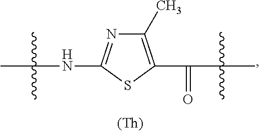 | − | − | + | + |
| 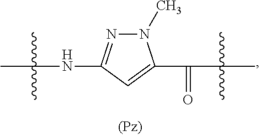 [SMILES: *Nc1cc(C(*)=O)n(C)n1] | − | − | + | + |
| 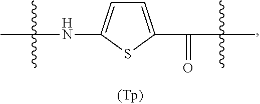 [SMILES: *Nc1ccc(C(*)=O)s1] | − | − | + | + |
| 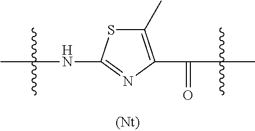 [SMILES: *Nc1nc(C(*)=O)c(C)s1] | + | − | − | − |
| 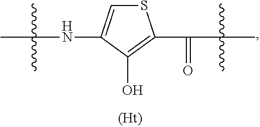 [SMILES: *Nc1csc(C(*)=O)c1O] | − | − | − | + |
| 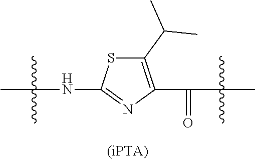 [SMILES: *Nc1nc(C(*)=O)c(C(C)C)s1] | + | − | − | − |
| 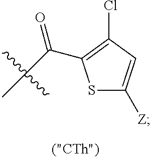 | − | − | − | + |
| 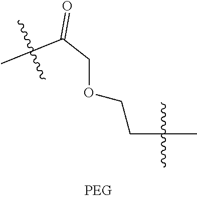 [SMILES: *CCOCC(*)=O] | − | + | + | + |
| 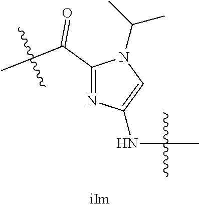 [SMILES: *Nc1cn(C(C)C)c(C(*)=O)n1] | + | − | − | − |
| 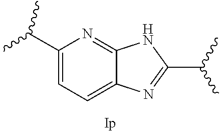 [SMILES: *c1ccc2nc(*)[nH]c2n1] | + | − | − | − |
| 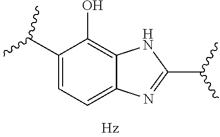 [SMILES: *c1nc2ccc(*)c(O)c2[nH]1] | − | − | − | + |
| 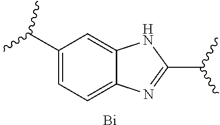 | − | − | − | + |
| 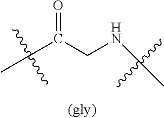 [SMILES: *NCC(*)=O] | − | − | − | − |
| 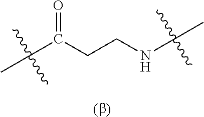 [SMILES: *NCCC(*)=O] | − | − | + | + |
| 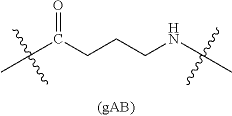 [SMILES: *NCCCC(*)=O] | − | − | + (as a part of the turn) | + (as a part of the turn) |
| 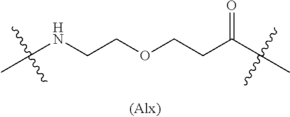 [SMILES: *NCCOCCC(*)=O] | − | + | − | − |
| 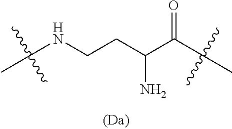 [SMILES: *NCCC(N)C(*)=O] | − | − | + | + |
| 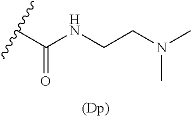 [SMILES: *C(=O)NCCN(C)C] | − | − | + | + |
| 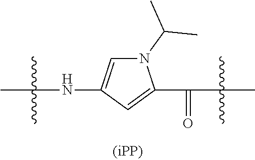 [SMILES: *Nc1cc(C(*)=O)n(C(C)C)c1] | − | − | + | + |
| 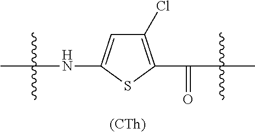 [SMILES: *Nc1cc(Cl)c(C(*)=O)s1 ; "(CTh)"] | + | + | − | − |
| 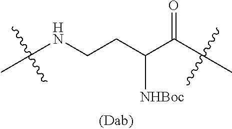 [SMILES: *NCCC(NC(=O)OC(C)(C)C)C(*)=O] | − | − | + | + |
| 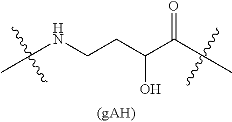 [SMILES: *NCCC(O)C(*)=O] | − | − | + | + |

| | |
|---|---|
| 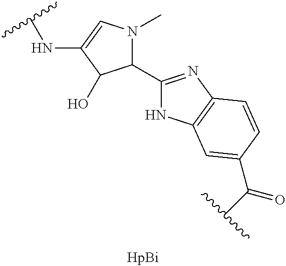 [SMILES: *NC1=CN(C)C(c2nc3ccc(C(*)=O)cc3[nH]2)C1O] | WW* (bind to two nucleotides with same selectivity as Hp-Py) |
| 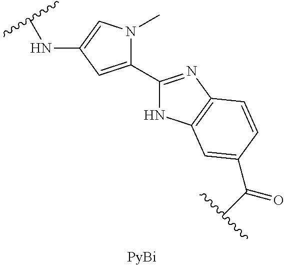 [SMILES: *Nc1cc(-c2nc3ccc(C(*)=O)cc3[nH]2)n(C)c1] | WW* (bind to two nucleotides with same selectivity as Py-Py) |
| 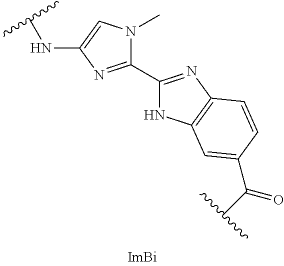 [SMILES: *Nc1cn(C)c(-c2nc3ccc(C(*)=O)cc3[nH]2)n1] | GW* (bind to two nucleotides with same selectivity as Im-Py) |

*The subunit HpBi, ImBi, and PyBi function as a conjugate of two monomer subunits and bind to two
nucleotides. The binding property of HpBi, ImBi, and PyBi corresponds to Hp-Py, Im-Py, and Py-Py
respectively.

*The subunit HpBi, ImBi, and PyBi function as a conjugate of two monomer subunits and bind to two nucleotides. The binding property of HpBi, ImBi, and PyBi corresponds to Hp-Py, Im-Py, and Py-Py respectively.

TABLE 1B
Base pairing for hairpin polyamide.

| | G•C | C•G | T•A | A•T |
|---|---|---|---|---|
| Im/β | + | − | − | − |
| β/Im | − | + | − | − |
| Py/β | − | − | + | + |
| β/Py | − | − | + | + |
| β/β | − | − | + | + |
| Py/Py | − | − | + | + |
| Im/Im | − | − | − | − |
| Im/Py | + | − | − | − |
| Py/Im | − | + | − | − |
| Th/Py | − | − | + | − |
| Py/Th | − | − | − | + |
| Th/Im | + | − | − | − |
| Im/Th | − | + | − | − |
| β/Th | − | − | + | − |
| Th/β | − | − | − | + |
| Hp/Py, | − | − | + | − |
| Py/Hp, | − | − | − | + |
| Hp/Im | + | − | − | − |
| Im/Hp | − | + | − | − |
| Tn/Py | − | − | + | + |
| Py/Tn, | − | − | + | + |
| Ht/Py, | − | − | + | + |
| Py/Ht, | − | − | + | + |
| Bi/Py, | − | − | + | + |
| Py/Bi, | − | − | + | + |
| β/Bi | − | − | + | + |
| Bi/β | − | − | + | + |
| Bi/Im, | − | − | + | + |
| Im/Bi, | − | − | + | − |
| Tp/Py, | − | − | + | + |
| Py/Tp, | − | − | + | + |
| β/Tp | − | − | + | + |
| Tp/β | − | − | + | + |
| Tp/Im, | − | + | − | − |
| Im/Tp | + | − | − | − |
| Tp/Tp | − | − | + | + |
| Tp/Tn | − | − | + | + |
| Tn/Tp | − | − | + | + |
| Hz/Py, | − | − | + | − |
| Py/Hz, | − | − | − | + |
| Ip/Py | + | − | − | − |
| Py/Ip, | − | + | − | − |
| Bi/Hz, | − | − | + | + |
| Hz/Bi, | − | − | + | + |
| Bi/Bi | − | + | + | + |
| Th/Py, | − | − | + | + |
| Py/Th | − | − | + | + |
| Im/gAB | + | − | − | − |
| gAB/Im | − | + | − | − |
| Py/gAB | + | − | − | − |
| gAB/Py | − | + | − | − |
| gAB/β | − | − | + | + |
| β/gAB | − | − | + | + |
| Im/Dp | + | − | − | − |
| Dp/Im | − | + | − | − |
| Py/Dp | − | − | + | + |
| Dp/Py | − | − | + | + |
| Dp/β | − | − | + | + |

Each of HpBi, ImBi, and PyBi can bind to two nucleotides and have binding properties corresponding to Hp-Py, Im-Py, and Py-Py respectively. HpBi, ImBi, and PyBi can be paired with two monomer subunits or with themselves in a hairpin structure to bind to two nucleotide pairs.

The monomer subunits of the polyamide can be strung together based on the paring principles shown in Table 1A and Table 1B. The monomer subunits of the polyamide can be strung together based on the paring principles shown in Table 1C and Table 1D.

Table 1C shows an example of the monomer subunits that can bind to the specific nucleotide. The first terminus can include a polyamide described having several monomer subunits stung together, with a monomer subunit selected from each row. For example, the polyamide can include Im-β-Py that binds to GAA, with Im selected from the first G column, p from the A column, and Py from the second A column. The polyamide can be any combinations that bind to the subunits of GAA, with a subunit selected from each column in Table 1C, wherein the subunits are strung together following the GAA order.

In addition, the polyamide can also include a partial or multiple sets of the five subunits, such as 1.5, 2, 2.5, 3, 3.5, or 4 sets of the three subunits. The polyamide can include 3, 4, 5, 6, 7, 8, 9, 10, 12, 14, and 16 monomer subunits. The multiple sets can be joined together by W. In addition to the five subunits or ten subunits, the polyamide can also include 1-4 additional subunits that can link multiple sets of the five subunits.

The polyamide can include monomer subunits that bind to 2, 3, 4, or 5 nucleotides of GAA. For example, the polyamide can bind to GA, AA, GAA, AAG, AGA, GAAG, AAGA, GAAGA or GAAGAA.

The polyamide can include monomer subunits that bind to 3, 4, 5, 6, 7, 8, 9, or 10 nucleotides of GAA repeats. The nucleotides can be joined by W.

The monomer subunit, when positioned as a terminal unit, does not have an amine, carbonyl, or a carboxylic acid group at the terminal. The amine or carboxylic acid group in the terminal is replaced by a hydrogen. For example, Py, when used as a terminal unit, is understood to have the structure of

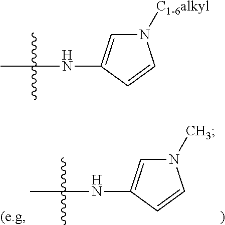

and Im, when positioned as a terminal unit, is understood to have the structure of

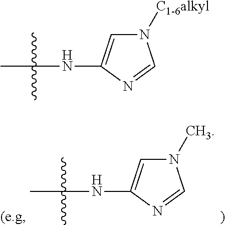

In addition, when Py or Im is used as a terminal unit, Py and Im can be respectively replaced by PyT

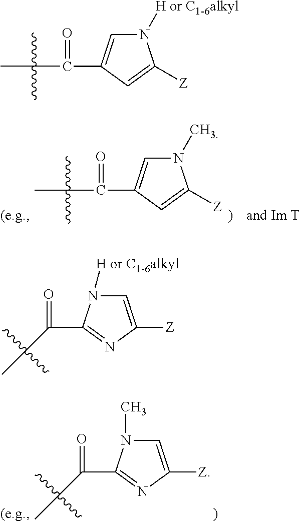

The linear polyamide can have nonlimiting examples including but not limited s-Py-Im, Im-Py-β-Im-Py-β-Im-Py, Im-Py-β-Im-Py-Py-Im-β, Im-Py-Py-Im-Py-β-Im-β, and any combinations thereof.

TABLE 1C
Examples of monomer subunits in a linear
polyamide that binds to GAA.

| Nucleotide | G | A | A |
|---|---|---|---|
| Subunit that selectively | Im or ImT | Py | Py |
| binds to nucleotide | iIm or iImT | Th | Th |
| | PEG | Pz | Pz |
| | CTh | Tp | Tp |
| | Nt | PEG | PEG |
| | iPTA | β | β |
| | Ip | iPP | iPP |
| | CTh | Da | Da |
| | | Dp | Dp |
| | | Dab | Dab |
| | | gAH | gAH |

The DNA-binding moiety can also include a hairpin polyamide having subunits that are strung together based on the pairing principle shown in Table 1B. Table 1D shows some examples of the monomer subunit pairs that selectively bind to the nucleotide pair. The hairpin polyamide can include 2n monomer subunits (n is an integer in the range of 2-8), and the polyamide also includes a W in the center of the 2n monomer subunits. W can be —(CH₂)ₙ—NR¹—(CH₂)_{b}—, —(CH₂)ₙ—, —(CH₂)ₙ—O—(CH₂)_{b}—, —(CH₂)ₙ—CH(NHR¹)—, —(CH₂)—CH(NHR¹)—, —(CR²R³)ₐ— or —(CH₂)ₙ—CH(NR¹₃)⁺—(CH₂)_{b}—, wherein each a is independently an integer between 2 and 4; R¹ is H, an optionally substituted C₁₋₆ alkyl, an optionally substituted C₃₋₁₀ cycloalkyl, an optionally substituted C₆₋₁₀ aryl, an optionally substituted 4-10 membered heterocyclyl, or an optionally substituted 5-10 membered heteroaryl; each R² and R³ are independently H, halogen, OH, NHAc, or C₁₋₄ alky. In some embodiments, W is —(CH₂)—CH(NH₃)⁺—(CH₂)— or —(CH₂)—CH₂CH(NH₃)⁺—. In some embodiments, R¹ is H. In some embodiments, R¹ is C₁₋₆ alkyl optionally substituted by 1-3 substituents selected from —C(O)-phenyl. In some embodiments, W is —(CR²R³)—(CH₂)ₙ— or —(CH₂)ₙ—(CR²R³)—(CH₂)_{b}—, wherein each a is independently 1-3, b is 0-3, and each R² and R³ are independently H, halogen, OH, NHAc, or C₁₋₄ alky. W can be an aliphatic amino acid residue shown in Table 4 such as gAB.

Because the target gene can include multiple repeats of GAA, the subunits can be strung together to bind at least two, three, four, five, six, seven, eight, nine, or ten nucleotides in one or more GAA repeat (e.g., GAAGAAGAAGAA). For example, the polyamide can bind to the GAA repeat by binding to a partial copy, a full copy, or a multiple repeats of GAA such as GA, AA, GAA, AAG, AGA, GAAG, AAGA, GAAGA or GAAGAA. For example, the polyamide can include Im-Py-β-W-Py-β-Py that binds to GAA and its complementary nucleotides on a double strand DNA, in which the Im/Py pair binds to the G·C, the Py/β pair binds to A·T, and the P/Py pair binds to G-A. In another example Im-Py-β-Im-W—β-Py-β-Py that binds to GAAG and its complementary nucleotides on a double strand DNA, in which the Im/Py pair binds to the G·C, the Py/P pair binds to A·T, the P/Py pair binds to G-A, and the Im/P pair binds to the G·C, W can be an aliphatic amino acid residue such as gAB or other appropriate spacers as shown in Table 4. In another example, Im-Py-β-Im-gAB-Im-Py binds to with a part of the complementary nucleotides (ACG) on the double strand DNA, in which Im binds to G, Py binds to A, Q/Py binds to the A·T, Im/Im binds to GC.

Some additional examples of the polyamide include but are not limited to Im-Py-Py-Im-gAB-Py-Im-Im-Py; Im-Py-Py-Im-gAB-Py-Im-Im-PyT; Im-Py-Py-Im-gAB-Py-Im-Im-3; Im-Py-Py-Im-gAB-Py-Im-Im-β-G; Im-β-β-Py-Im-gAB-Py-Im-Im-β; Im-β-Py-Im-gAB-Py-Im-Im-P-G; Im-P-Py-Im-gAB-Py-Im-Im-Py; Im-@3-Py-Im-gAB-Py-Im-Im-PyT; Py-Py-Im-3-gAB-Im-Py-Im-Im; Py-Py-Im-3-gAB-Im-Py-Im-ImT; Py-Py-Im-Py-gAB-Im-Py-Im-m; Py-Py-Im-Py-gAB-Im-Py-Im-ImT; Py-Py-Im-β-gAB-Im-β-Im-Im; Py-Py-Im-β-gAB-Im-β-Im-ImT; Py-Py-Im-Py-gAB-Im-P-Im-Im; Py-Py-Im-Py-gAB-Im-β-Im-ImT; Im-β-Py-gAB-Im-Im-Py; Im-P-Py-gAB-Im-Im-PyT; Im-β-Py-gAB-Im-Im-β; Im-β-Py-gAB-Im-Im-P-G; Im-Py-Py-gAB-Im-Im-β; Im-Py-Py-gAB-Im-Im-H-G; Im-Py-Py-gAB-Im-Im-Py; Im-Py-Py-gAB-Im-Im-PyT; Im-β-Py-gAB-Im-Im-Py; and Im-β-Py-gAB-Im-Im-PyT; wherein G may be hydrogen, alkyl, alkenyl, alkynyl, or —C(O)—R_{B}; and R_{B} may be a hydrogen, C₁-C₆ alkyl, C₁-C₆ alkenyl, or C₁-C₆ alkynyl group. In some embodiments, the hairpin polyamide has a structure of Im-Py-β-Im-gAB-Im-Py; Im-Py-β-Im-gAB-Im-Py-β-Im; Py-β-Im-gAB-Im-Py-β-Im; or R-Im-gAB-mm-Py-β-Im.

TABLE 1D
Examples of monomer pairs in a hairpin or
H-pin polyamide that binds to CAG or CTG.

| Nucleotide | G•C | A•T | A•T |
|---|---|---|---|
| Subunit pairs that selectively | Im/β | Py/β | Py/β |
| binds to nucleotide | Im/Py | β/Py | β/Py |
| | Th/Im | β/β | β/β |
| | Hp/Im | Py/Py | Py/Py |
| | Im/Bi | Py/Th | Py/Th |
| | Im/Tp | Th/β | Th/β |
| | Ip/Py | Py/Hp, | Py/Hp, |
| | Im/gAB | Tn/Py | Tn/Py |
| | Py/gAB | Py/Tn, | Py/Tn, |
| | Im/Dp | Ht/Py, | Ht/Py, |
| | | Py/Ht, | Py/Ht, |
| | | Bi/Py, | Bi/Py, |
| | | Py/Bi, | Py/Bi, |
| | | β/Bi | β/Bi |
| | | Bi/β | Bi/β |
| | | Tp/Py, | Tp/Py, |
| | | Py/Tp, | Py/Tp, |
| | | β/Tp | β/Tp |
| | | Tp/β | Tp/β |
| | | Tp/Tp | Tp/Tp |
| | | Tp/Tn | Tp/Tn |
| | | Tn/Tp | Tn/Tp |
| | | Py/Hz, | Py/Hz, |
| | | Bi/Hz, | Bi/Hz, |
| | | Hz/Bi, | Hz/Bi, |
| | | Bi/Bi | Bi/Bi |
| | | Th/Py, | Th/Py, |
| | | Py/Th | Py/Th |
| | | gAB/β | gAB/β |
| | | β/gAB | β/gAB |
| | | Py/Dp | Py/Dp |
| | | Dp/Py | Dp/Py |
| | | Dp/β | Dp/β |

Recognition of a nucleotide repeat or DNA sequence by two antiparallel polyamide strands depends on a code of side-by-side aromatic amino acid pairs in the minor groove, usually oriented N to C with respect to the 5′ to 3′ direction of the DNA helix. Enhanced affinity and specificity of polyamide nucleotide binding is accomplished by covalently linking the antiparallel strands. The “hairpin motif” connects the N and C termini of the two strands with a W (e.g., gamma-aminobutyric acid unit (gamma-turn)) to form a folded linear chain. The “H-pin motif” connects the antiparallel strands across a central or near central ring/ring pairs by a short, flexible bridge.

The DNA-binding moiety can also include a H-pin polyamide having subunits that are strung together based on the pairing principles shown in Table 1A and/or Table 1B. Table 1C shows some examples of the monomer subunit that selectively binds to the nucleotide, and Table 1D shows some examples of the monomer subunit pairs that selectively bind to the nucleotide pair. The h-pin polyamide can include 2 strands and each strand can have a number of monomer subunits (each strand can include 2-8 monomer subunits), and the polyamide also includes a bridge L₁ to connect the two strands in the center or near the center of each strand. At least one or two of the monomer subunits on each strand are paired with the corresponding monomer subunits on the other stand following the paring principle in Table 1D to favor binding of either G·C or C·G, A·T, or T·A pair, and these monomer subunit pairs are often positioned in the center, close to center region, at or close to the bridge that connects the two strands. In some instances, the H-pin polyamide can have all of the monomer subunits be paired with the corresponding monomer subunits on the antiparallel strand based on the paring principle in Table 1B and 1D to bind to the nucleotide pairs on the double strand DNA. In some instances, the H-pin polyamide can have a part of the monomer subunits (2, 3, 4, 5, or 6) be paired with the corresponding monomer subunits on the antiparallel strand based on the binding principle in Table 1B and 1D to bind to the nucleotide pairs on the double strand DNA, while the rest of the monomer subunit binds to the nucleotide based on the binding principle in Table 1A and 1C but does not pair with the monomer subunit on the antiparallel strand. The h-pin polyamide can have one or more overhanging monomer subunit that binds to the nucleotide but does not pair with the monomer subunit on the antiparallel strand.

Another polyamide structure that derives from the h-pin structure is to connect the two antiparallel strands at the end through a bridge, while only the two monomer subunits that are connected by the bridge form a pair that bind to the nucleotide pair G·C or C·G based on the binding principle in Table 1B/1D, but the rest of the monomer subunits on the strand form an overhang, bind to the nucleotide based on the binding principle in Table 1A and/or 1C and do not pair with the monomer subunit on the other strand.

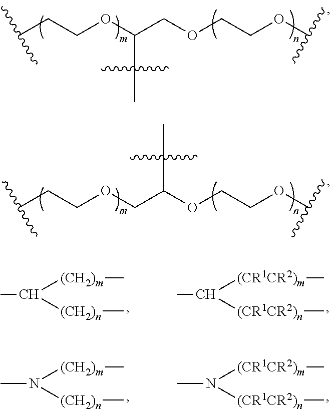

The bridge can be is a bivalent or trivalent group selected from

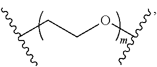

a C₁₋₁₀ alkylene, —NH—C₀₋₆ alkylene-C(O)—, —N(CH₃)—C₀₋₆ alkylene, and, —(CH₂)ₙ—NR¹—(CH₂)_{b}—, —(CH₂)ₙ—, —(CH₂)ₙ—O—(CH₂)_{b}—, —(CH₂)ₙ—CH(NHR¹)—, —(CH₂)ₙ—CH(NHR¹)—, —(CR²R³)ₙ— or —(CH₂)ₙ—CH(NR¹₃)—(CH₂)_{b}—, wherein m is an integer in the range of 0 to 10; n is an integer in the range of 0 to 10; each a is independently an integer between 2 and 4; R¹ is H, an optionally substituted C₁₋₆ alkyl, an optionally substituted C₃₋₁₀ cycloalkyl, an optionally substituted C₆₋₁₀ aryl, an optionally substituted 4-10 membered heterocyclyl, or an optionally substituted 5-10 membered heteroaryl; each R² and R³ are independently H, halogen, OH, NHAc, or C₁₋₄ alky. In some embodiments, W is —(CH₂)—CH(NH₃)⁺—(CH₂)— or —(CH₂)—CH₂CH(NH₃)⁺—. In some embodiments, R¹ is H. In some embodiments, R¹ is C₁₋₆ alkyl optionally substituted by 1-3 substituents selected from —C(O)-phenyl. In some embodiments, L₁ is —(CR²R³)—(CH₂)ₙ— or —(CH₂)ₐ—(CR²R³)—(CH₂)_{b}—, wherein each a is independently 1-3, b is 0-3, and each R² and R³ are independently H, halogen, OH, NHAc, or C₁₋₄ alky. L₁ can be a C₂₋₉ alkylene or (PEG)₂-8.

Some additional examples of the polyamide include but are not limited to Im-Py-Py-Im (Linked in the middle—either position 2 or 3) to Py-Py-Py-Py, Im-Py-Py-Im (Linked in the middle—position 3 py and Py) to Im-Py-β-Py-Py, Im-Py-β-Im (linked to the bolded position) Im-Py; Im-Py-β-Im (linked in the middle, either position 2 or 3) Im-Py-b-Im; Py-β-Im (linked to the middle position bolded) Im-Py-β-Im; or β-Im (linked at bolded position) Im-Py-β-Im.

### Second Terminus—Regulatory Binding Moiety

In certain embodiments, the regulatory molecule is chosen from a nucleosome remodeling factor (“NURF”), a bromodomain PHD finger transcription factor (“BPTF”), a ten-eleven translocation enzyme (“TET”), methylcytosine dioxygenase (“TET1”), a DNA demethylase, a helicase, an acetyltransferase, and a histone deacetylase (“HDAC”).

In some embodiments, the protein-binding moiety binds to the regulatory molecule that is selected from the group consisting of a CREB binding protein (“CBP”), a P300, an O-linked β-N-acetylglucosamine-transferase-(OGT-), a P300-CBP-associated-factor-(PCAF-), histone methyltransferase, histone demethylase, chromodomain, a cyclin-dependent-kinase-9-(CDK9-), a nucleosome-remodeling-factor-(NURF-), a bromodomain-PHD-finger-transcription-factor-(BPTF-), a ten-eleven-translocation-enzyme-(TET-), a methylcytosine-dioxygenase-(TET1-), histone acetyltransferase (HAT), a histone deacetylase (HDAC), a host-cell-factor-1(HCF1-), an octamer-binding-transcription-factor-(OCT1-), a β-TEFb-, a cyclin-T1-, a PRC2-, a DNA-demethylase, a helicase, an acetyltransferase, a histone-deacetylase, methylated histone lysine protein.

In some embodiments, the second terminus comprises a moiety that binds to an O-linked β-N-acetylglucosamine-transferase (OGT), or CREB binding protein (CBP). In some embodiments, the protein binding moiety is a residue of a compound that binds to an O-linked β-N-acetylglucosamine-transferase (OGT), or CREB binding protein (CBP).

In some embodiments, the second terminus comprises a bromodomain binding moiety. In some embodiments, the bromodomain binding moiety is a BRD2, BRD3, BRD4, or BRDT binding moiety. In some embodiments, the bromodomain binding moiety is a BRD4 binding moiety.

In some embodiments, the regulatory molecule is a bromodomain-containing protein chosen from BRD2, BRD3, BRD4, and BRDT.

In some embodiments, the regulatory molecule is BRD4. In certain embodiments, the recruiting moiety is a BRD4 activator.

In certain embodiments, the regulatory molecule modulates the rearrangement of histones.

In certain embodiments, the regulatory molecule modulates the glycosylation, phosphorylation, alkylation, or acylation of histones.

In certain embodiments, the regulatory molecule is a transcription factor.

In certain embodiments, the regulatory molecule is an RNA polymerase.

In certain embodiments, the regulatory molecule is a moiety that regulates the activity of RNA polymerase.

In certain embodiments, the recruiting moiety binds to the regulatory molecule but does not inhibit the activity of the regulatory molecule. In certain embodiments, the recruiting moiety binds to the regulatory molecule and inhibits the activity of the regulatory molecule. In certain embodiments, the recruiting moiety binds to the regulatory molecule and increases the activity of the regulatory molecule.

In certain embodiments, the recruiting moiety binds to the active site of the regulatory molecule.

In certain embodiments, the recruiting moiety binds to a regulatory site of the regulatory molecule.

The binding affinity between the regulatory protein and the second terminus can be adjusted based on the composition of the molecule or type of protein. In some embodiments, the second terminus binds the regulatory molecule with an affinity of less than about 600 nM, about 500 nM, about 400 nM, about 300 nM, about 250 nM, about 200 nM, about 150 nM, about 100 nM, or about 50 nM. In some embodiments, the second terminus binds the regulatory molecule with an affinity of less than about 300 nM. In some embodiments, the second terminus binds the regulatory molecule with an affinity of less than about 200 nM.

In some embodiments, the polyamide is capable of binding the DNA with an affinity of greater than about 200 nM, about 150 nM, about 100 nM, about 50 nM, about 10 nM, or about 1 nM. In some embodiments, the polyamide is capable of binding the DNA with an affinity in the range of about 1-600 nM, 10-500 nM, 20-500 nM, 50-400 nM, 100-300 nM, or 50-200 nM.

In some embodiments, the second terminus comprises a diazine or diazepine ring, wherein the diazine or diazepine ring is fused with a C₆₋₁₀ aryl or a 5-10 membered heteroaryl ring comprising one or more heteroatom selected from S, N and O. In some embodiments, the second terminus comprises an optionally substituted bicyclic or tricyclic structure. In some embodiments, the optionally substituted bicyclic or tricyclic structure comprises a diazepine ring fused with a thiophene ring. In some embodiments, the second terminus comprises an optionally substituted bicyclic structure, wherein the bicyclic structure comprises a diazepine ring fused with a thiophene ring.

In some embodiments, the second terminus comprises a compound having the structure of Formula (9-A), or a pharmaceutically acceptable salt thereof:

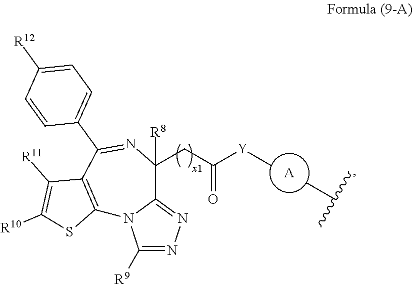

wherein;
Ring A is absent or an optionally substituted 6-membered monocyclic aryl or heteroaryl;
Y is —NH— or —O—;
R⁸ is hydrogen or C₁₋₆ alkyl;
R⁹, R¹⁰, and R″ are each independently selected from hydrogen, optionally substituted C₁₋₆ alkyl, C₁₋₆ haloalkyl, or C₁₋₆ hydroxyalkyl;
R¹² is selected from hydrogen, halogen, —NO₂, —CN, optionally substituted aryl, optionally substituted C₁₋₂₀ alkyl, C₁₋₂₀ heteroalkyl, C₁₋₆ haloalkyl, or C₁₋₆ hydroxyalkyl;
or R¹² is —NR^{A}R^{B}, wherein
   R^{A} and R^{B} are each independently hydrogen, optionally substituted C₁₋₂₀ alkyl or C₁-20 heteroalkyl; and
x₁ is an integer from 1-6.

In some embodiments, Ring A is an optionally substituted 6-membered monocyclic aryl or heteroaryl, each of which is optionally substituted with alkyl, amino, halogen, hydroxy, hydroxyalkyl, or PEG. In some embodiments Ring A is phenyl. In some embodiments, Ring A is 6-membered monocyclic heteroaryl. In some embodiments, Ring A is pyridine or pyrimidine.

In some embodiments, Ring A is absent.

In some embodiments, Y is —NH—. In some embodiments, Y is —O—.

In some embodiments, R⁸ is hydrogen.

In some embodiments, R⁹, R¹⁰, and R″ are each independently selected from optionally substituted C₁₋₆ alkyl, C₁₋₆ haloalkyl, or C₁₋₆ hydroxyalkyl. In some embodiments, R⁹, R¹⁰, and R″ are each independently selected from optionally substituted C₁₋₆ alkyl. In some embodiments, In some embodiments, R⁹, R¹⁰, and R″ are each independently methyl, ethyl, or propyl. In some embodiments, In some embodiments, R⁹, R¹⁰, and R″ are each independently methyl.

In some embodiments, R″ is selected from hydrogen, halogen, optionally substituted C₁₋₆ alkyl, C₁₋₆ haloalkyl, or C₁₋₆ hydroxyalkyl. In some embodiments, R″ is bromo, chloro, or fluoro.

In some embodiments, R″ is —NR^{A}R^{B}, wherein R^{A} and R^{B} are each independently hydrogen, optionally substituted C₁₋₆ alkyl.

In some embodiments, x₁ is an integer from 1-5, 1-4, 1-3, or 1-2. In some embodiments, x₁ is 1. In some embodiments, x₁ is 2.

In some embodiments, the second terminus comprises a compound having the structure of Formula (9-B), or a pharmaceutically acceptable salt thereof:

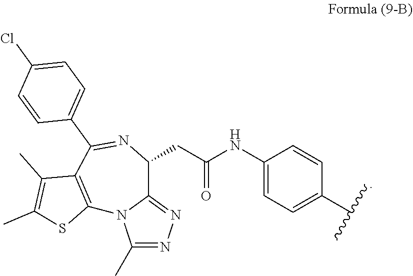 [SMILES: *c1ccc(NC(=O)C[C@H]2N=C(c3ccc(Cl)cc3)c3c(sc(C)c3C)-n3c(C)nnc32)cc1]

In some embodiments, the second terminus comprises a compound of Formula (10-A), or a pharmaceutically acceptable salt thereof:

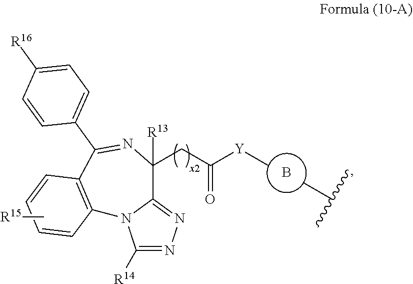

wherein,
Ring B is absent or an optionally substituted 5-6-membered monocyclic aryl or heteroaryl or 4-8-membered heterocycle;
Y is —NH— or —O—;
R¹³ is selected from hydrogen or optionally substituted C₁-C₆ alkyl;
R¹⁴ and R¹⁵ are each independently selected from hydrogen, optionally substituted C₁₋₆ alkyl, C₁₋₆ haloalkyl, or C₁₋₆ hydroxyalkyl;
R¹⁶ is selected from hydrogen, halogen, —NO₂, —CN, optionally substituted aryl, optionally substituted C₁₋₂₀ alkyl, C₁-20 heteroalkyl, C₁₋₆ haloalkyl, or C₁₋₆ hydroxyalkyl;
or R¹⁶ is —NR^{A}R^{B}, wherein
   R^{A} and R^{B} are each independently hydrogen, optionally substituted C₁₋₂₀ alkyl or C₁-20 heteroalkyl; and
x₂ is an integer from 1-6.

In some embodiments, Ring B is an optionally substituted 6-membered monocyclic aryl or heteroaryl, each of which is optionally substituted with alkyl, amino, halogen, hydroxy, hydroxyalkyl, or PEG. In some embodiments Ring B is phenyl. In some embodiments, Ring B is 6-membered monocyclic heteroaryl. In some embodiments, Ring B is pyridine or pyrimidine.

In some embodiments, Ring B is absent.

In some embodiments, Y is —NH—. In some embodiments, Y is —O—.

In some embodiments, R¹³ is hydrogen.

In some embodiments, R¹⁴ and R¹⁵ are each independently selected from optionally substituted C₁. 6 alkyl, C₁₋₆ haloalkyl, or C₁₋₆ hydroxyalkyl. In some embodiments, R¹⁴ and R¹⁵ are each independently selected from optionally substituted C₁₋₆ alkyl. In some embodiments, In some embodiments, R¹⁴ and R¹⁵ are each independently methyl, ethyl, or propyl. In some embodiments, In some embodiments, R¹⁴ and R¹⁵ are each independently methyl.

In some embodiments, R¹⁶ is selected from hydrogen, halogen, optionally substituted C₁₋₆ alkyl, C₁₋₆ haloalkyl, or C₁₋₆ hydroxyalkyl. In some embodiments, R¹⁶ is bromo, chloro, or fluoro.

In some embodiments, R¹⁶ is —NR^{A}R^{B}, wherein R^{A} and R^{B} are each independently hydrogen, optionally substituted C₁₋₆ alkyl.

In some embodiments, x₂ is an integer from 1-5, 1-4, 1-3, or 1-2. In some embodiments, x₂ is 1. In some embodiments, x₂ is 2.

In some embodiments, the second terminus comprises a compound having the structure of Formula (10-B), or a pharmaceutically acceptable salt thereof:

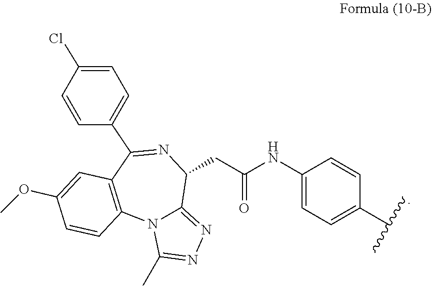 [SMILES: *c1ccc(NC(=O)C[C@H]2N=C(c3ccc(Cl)cc3)c3cc(OC)ccc3-n3c(C)nnc32)cc1]

In some embodiments, the second terminus comprises a compound having the structure of Formula (11), or a pharmaceutically acceptable salt thereof:

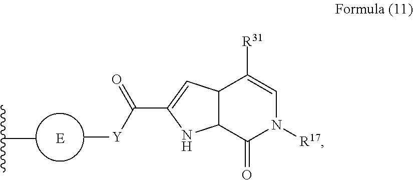

wherein,
Ring E is absent or an optionally substituted 5-6-membered monocyclic aryl or heteroaryl or 4-8-membered heterocycle;
Y is —NH— or —O—;
R¹⁷ is hydrogen or —C₁-C₆ alkyl; and
R³¹ is hydrogen or a substituted monocyclic aryl or heteroaryl.

In some embodiments, the second terminus comprises a compound having the structure of Formula (11-A), or a pharmaceutically acceptable salt thereof:

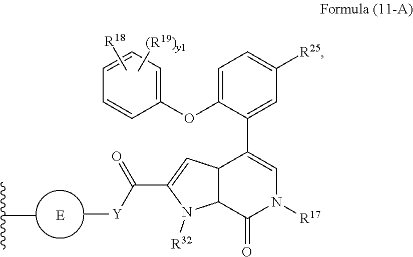

wherein,
Ring E is absent or an optionally substituted 5-6-membered monocyclic aryl or heteroaryl or 4-8-membered heterocycle;
Y is —NH— or —O—;
R¹⁷ is hydrogen or —C₁-C₆ alkyl;
R¹⁸ and R¹⁹ are each independently hydrogen, halogen, —CN, —NO₂, optionally substituted —C₁-C₆ alkyl, C₁-C₆ haloalkyl or C₁-C₆ hydroxyalkyl;
or R¹⁸ is —NR^{A}R^{B}, wherein
   R^{A} and R^{B} are each independently hydrogen, optionally substituted C₁₋₆ alkyl or C₁₋₆ heteroalkyl;
R²⁵ is optionally substituted optionally substituted C₁₋₆ alkyl, C₁₋₆ heteroalkyl, C₁₋₆ alkenyl, C₁₋₆ alkynyl, C₁₋₆ hydroxyalkyl, or —NHSO₂R^{A};
R³² is hydrogen or an optionally substituted C₁₋₆ alkyl; and
y₁ is 1-3.

In some embodiments, Ring E is an optionally substituted 5 or 6-membered monocyclic aryl or heteroaryl, wherein each is optionally substituted with each of which is optionally substituted with alkyl, amino, halogen, hydroxy, hydroxyalkyl, or PEG.

In some embodiments, Ring E is phenyl. In some embodiments, Ring E is a 6-membered heteroaryl. In some embodiments, Ring E is pyridine, pyrazine, or triazine. In some embodiments, Ring E is pyridine. In some embodiments, Ring E is pyrazine. In some embodiments, Ring E is triazine. In some embodiments, Ring E is a 5-membered heteroaryl. In some embodiments, Ring E is a pyrazole. In some embodiments, Ring E is a triazole, pyrrole, imidazole, oxazole, oxadiazole, thiazole, or thiadiazole. In some embodiments, Ring E is a triazole. In some embodiments, Ring E is an imidazole or pyrrole. In some embodiments, an oxazole or oxadiazole. In some embodiments, Ring E is a thiazole or thiadiazole.

In some embodiments, Ring E is absent.

In some embodiments, R¹⁷ is hydrogen. In some embodiments, R¹⁷ is C₁-C₆ alkyl. In some embodiments, R¹⁷ is methyl, ethyl, propyl. In some embodiments, R¹⁷ is methyl.

In some embodiments, R¹⁸ and R¹⁹ are each independently hydrogen, —CN, or —NO₂. In some embodiments, R¹⁸ and R¹⁹ are each independently halogen or optionally substituted —C₁-C₆ alkyl. In some embodiments, R¹⁸ and R¹⁹ are each independently -bromo, chloro, fluoro, methyl, or ethyl. In some embodiments, R¹⁸ and R¹⁹ are each independently fluoro or methyl.

In some embodiments, R²⁵ is optionally substituted optionally substituted C₁₋₆ alkyl, C₁₋₆ heteroalkyl, C₁₋₆ alkenyl, C₁₋₆ alkynyl, or C₁₋₆ hydroxyalkyl, each of which is optionally substituted with amido, alkyl, alkynyl, azido, amino, halogen, haloalkyl, hydroxy, nitro, oxo (═O), phosphorous hydroxide, or PEG.

In some embodiments, R²⁵ is optionally substituted optionally substituted C₁₋₆ alkyl, C₁₋₆ heteroalkyl, or C₁-C₆ hydroxyalkyl. In some embodiments, R²⁵ is C₁₋₆ alkyl or C₁₋₆ heteroalkyl, each or which optionally substituted with —CN, —NH₂, —N₃, —OH, CF₃, or —OP(═O)(OH)₂.

In some embodiments, R²⁵ is —NHSO₂R^{A}. In some embodiments, R²⁵ is —NHSO₂Et. In some embodiments, R²⁵ is —NHSO₂Me.

In some embodiments, R³² is C₁₋₆ alkyl, optionally substituted with haloalkyl, phosphorous hydroxide. In some embodiments, R³² is C₁₋₆ alkyl substituted with —OP(═O)(OH)₂. In some embodiments, R³² is unsubstituted C₁₋₆ alkyl. In some embodiments, R³² is methyl, ethyl, or tributyl. In some embodiments, R³² is hydrogen.

In some embodiments, y₁ is 1. In some embodiments, y₁ is 2. In some embodiments, y₁ is 3.

In some embodiments, the second terminus comprises a compound having the structure of Formula (11-B), or a pharmaceutically acceptable salt thereof:

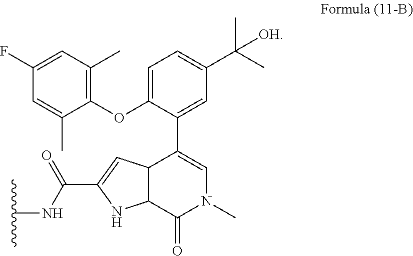 [SMILES: *NC(=O)C1=CC2C(c3cc(C(C)(C)O)ccc3Oc3c(C)cc(F)cc3C)=CN(C)C(=O)C2N1]

In some embodiments, the second terminus comprises a compound having the structure of Formula (11-C), or a pharmaceutically acceptable salt thereof:

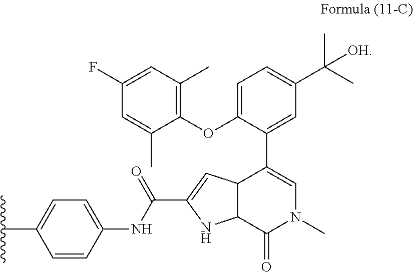 [SMILES: *c1ccc(NC(=O)C2=CC3C(c4cc(C(C)(C)O)ccc4Oc4c(C)cc(F)cc4C)=CN(C)C(=O)C3N2)cc1]

In some embodiments, the second terminus comprises a compound having the structure of Formula (11-D), or a pharmaceutically acceptable salt thereof:

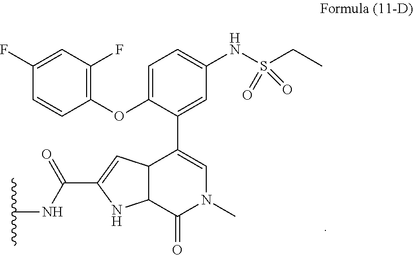 [SMILES: *NC(=O)C1=CC2C(c3cc(NS(=O)(=O)CC)ccc3Oc3ccc(F)cc3F)=CN(C)C(=O)C2N1]

In some embodiments, the second terminus comprises a compound having the structure of Formula (11-E), or a pharmaceutically acceptable salt thereof:

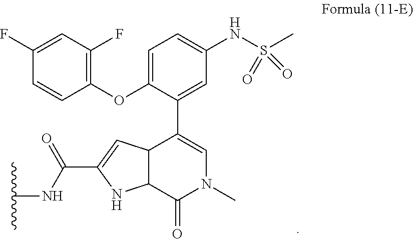 [SMILES: *NC(=O)C1=CC2C(c3cc(NS(C)(=O)=O)ccc3Oc3ccc(F)cc3F)=CN(C)C(=O)C2N1]

In some embodiments, the second terminus comprises a compound having the structure of Formula (11-F), or a pharmaceutically acceptable salt thereof:

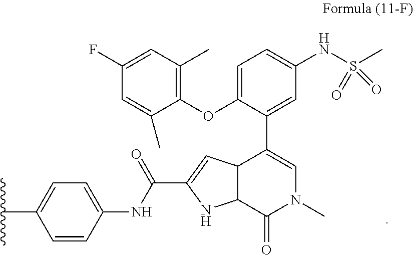 [SMILES: *c1ccc(NC(=O)C2=CC3C(c4cc(NS(C)(=O)=O)ccc4Oc4c(C)cc(F)cc4C)=CN(C)C(=O)C3N2)cc1]

In some embodiments, the second terminus comprises a compound having the structure of Formula (11-G), or a pharmaceutically acceptable salt thereof:

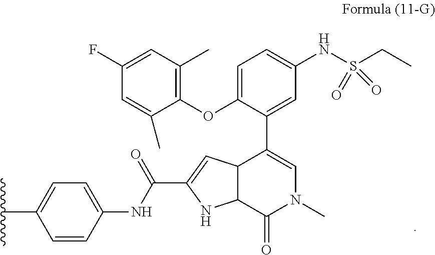 [SMILES: *c1ccc(NC(=O)C2=CC3C(c4cc(NS(=O)(=O)CC)ccc4Oc4c(C)cc(F)cc4C)=CN(C)C(=O)C3N2)cc1]

In some embodiments, the second terminus comprises a compound having the structure of Formula (12-A), or a pharmaceutically acceptable salt thereof:

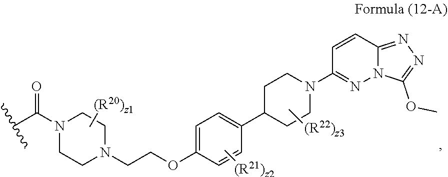

wherein,
each R²⁰, R²¹, and R²² is independently hydrogen, halogen, optionally substituted C₁-C₆ alkyl, C₁-C₆ haloalkyl or C₁-C₆ hydroxyalkyl; and
each z₁, z₂, and z₃ is independently 1-4.

In some embodiments, each R²⁰, R²¹, and R²² is independently an optionally substituted C₁-C₆ alkyl, C₁-C₆ haloalkyl or C₁-C₆ hydroxyalkyl. In some embodiments, each R²⁰, R²¹, and R²² is independently methyl, ethyl, or propyl. In some embodiments, R²⁰, R²¹, and R²² is independently halogen. In some embodiments, R²⁰, R²¹, and R²² is independently hydrogen.

In some embodiments, each z₁, z₂, and z₃ is independently an integer from 1-3 or 1-2. In some embodiments, each z₁, z₂, and z₃ is independently 1. In some embodiments, each z₁, z₂, and z₃ is independently 2. In some embodiments, each z₁, z₂, and z₃ is independently 3.

In some embodiments, the second terminus comprises a compound having the structure of Formula (12-B), or a pharmaceutically acceptable salt thereof:

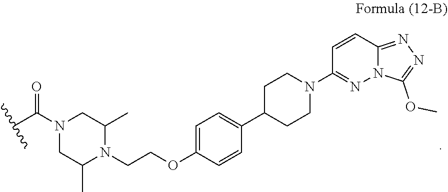 [SMILES: *C(=O)N1CC(C)N(CCOc2ccc(C3CCN(c4ccc5nnc(OC)n5n4)CC3)cc2)C(C)C1]

In some embodiments, the second terminus comprises a compound having the structure of Formula (12-C), or a pharmaceutically acceptable salt thereof:

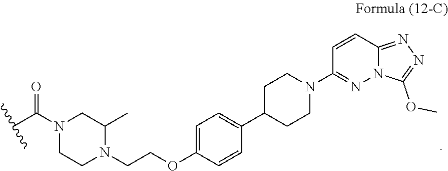 [SMILES: *C(=O)N1CCN(CCOc2ccc(C3CCN(c4ccc5nnc(OC)n5n4)CC3)cc2)C(C)C1]

In some embodiments, the second terminus comprises a compound having the structure of Formula (13-A), or a pharmaceutically acceptable salt thereof:

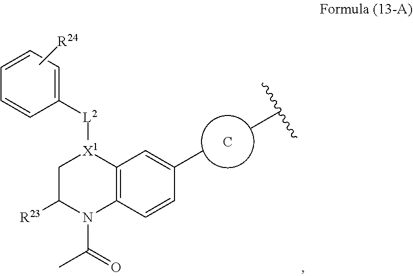

wherein;
Ring C is absent or an optionally substituted monocyclic 6-membered aryl or heteroaryl;
X¹ is CH or N;
L² is —NR^{D}— or —CR^{D}H—
R²³ is C₁-C₆ alkyl or C₃-C₆ cycloalkyl; and
R²⁴ is halogen, alkyl, hydroxyalkyl, haloalkyl; optionally substituted C₁-C₆ alkyl, C₁-C₆ haloalkyl or C₁-C₆ hydroxyalkyl; and
R^{D} is hydrogen or C₁₋₃ alkyl.

In some embodiments, In some embodiments, Ring C is a optionally substituted 6-membered monocyclic aryl or heteroaryl, wherein each is optionally substituted with each of which is optionally substituted with alkyl, amino, halogen, hydroxy, hydroxyalkyl, or PEG. In some embodiments Ring C is phenyl. In some embodiments, Ring C is 6-membered monocyclic heteroaryl. In some embodiments, Ring C is pyridine or pyrimidine.

In some embodiments, In some embodiments, Ring C is

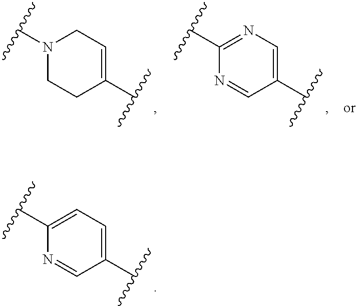 [SMILES: *C1=CCN(*)CC1 ; *c1cnc(*)nc1 ; *c1ccc(*)nc1]

In some embodiments, Ring C is absent.

In some embodiments, X¹ is CH. In some embodiments, X¹ is N.

In some embodiments, L² is —NR^{D}—. In some embodiments, L² is —NH—. In some embodiments, L² is —CR^{D}H. In some embodiments, L² is —CH₂—.

In some embodiments, R²³ is methyl, ethyl, or propyl. In some embodiments, R²³ is methyl. In some embodiments, R²³ is ethyl. In some embodiments, R²³ is propyl. In some embodiments, R²³ is cyclopropyl.

In some embodiments, R²⁴ is alkyl, hydroxyalkyl, haloalkyl; optionally substituted C₁-C₆ alkyl, C₁-C₆ haloalkyl or C₁-C₆ hydroxyalkyl. In some embodiments, R²⁴ is hydroxyalkyl. In some embodiments, R²⁴ is halogen. In some embodiments, R²⁴ is bromo, chloro, or fluoro.

In some embodiments, the second terminus comprises a compound having the structure of Formula (13-B), or a pharmaceutically acceptable salt thereof:

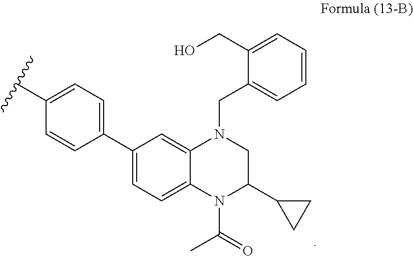 [SMILES: *c1ccc(-c2ccc3c(c2)N(Cc2ccccc2CO)CC(C2CC2)N3C(C)=O)cc1]

In some embodiments, the second terminus comprises a compound having the structure of Formula (13-C), or a pharmaceutically acceptable salt thereof:

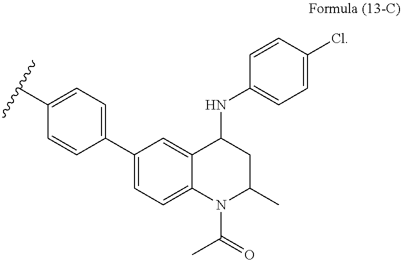 [SMILES: *c1ccc(-c2ccc3c(c2)C(Nc2ccc(Cl)cc2)CC(C)N3C(C)=O)cc1]

In some embodiments, the second terminus is selected from:

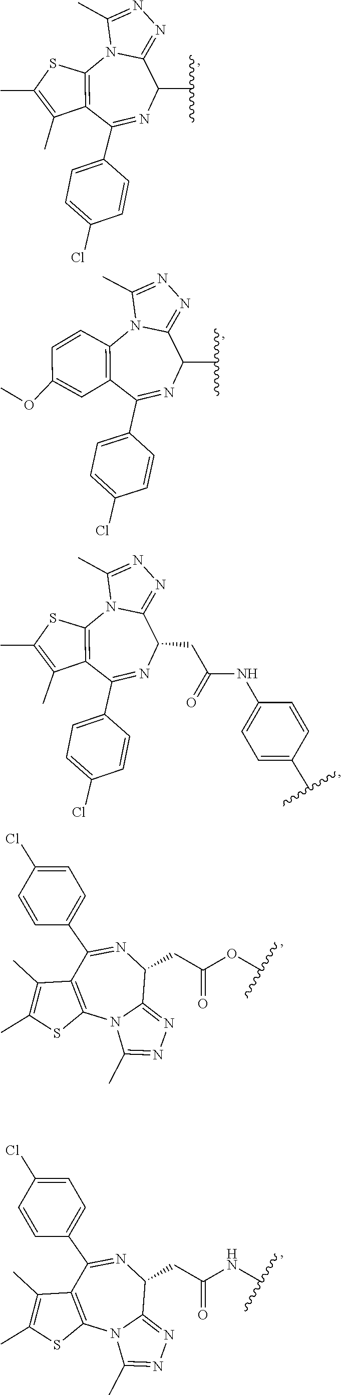

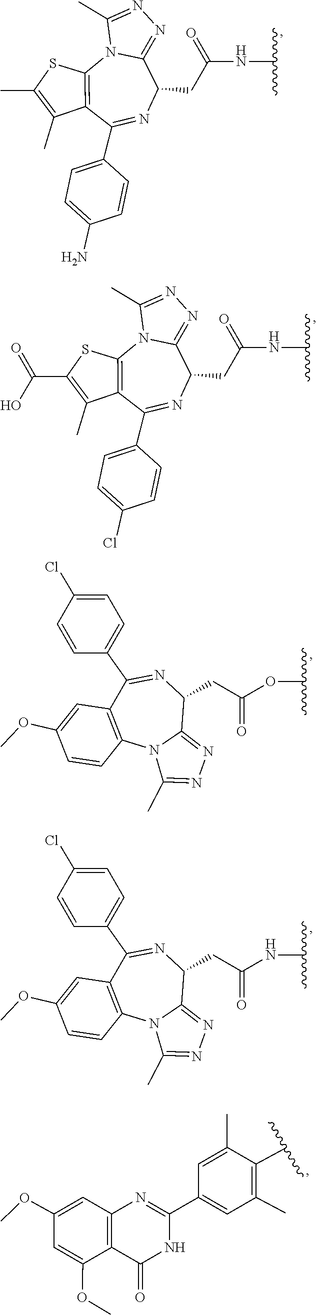

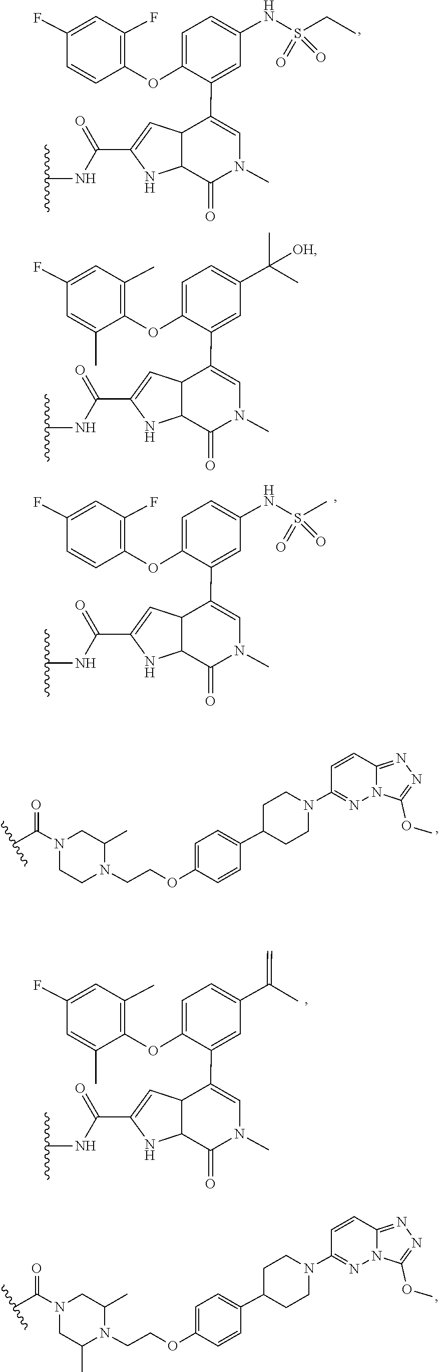

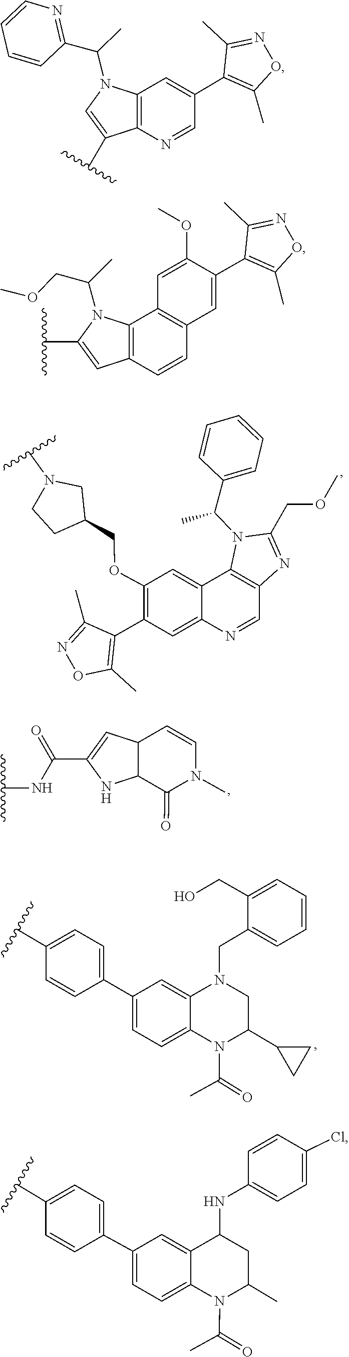

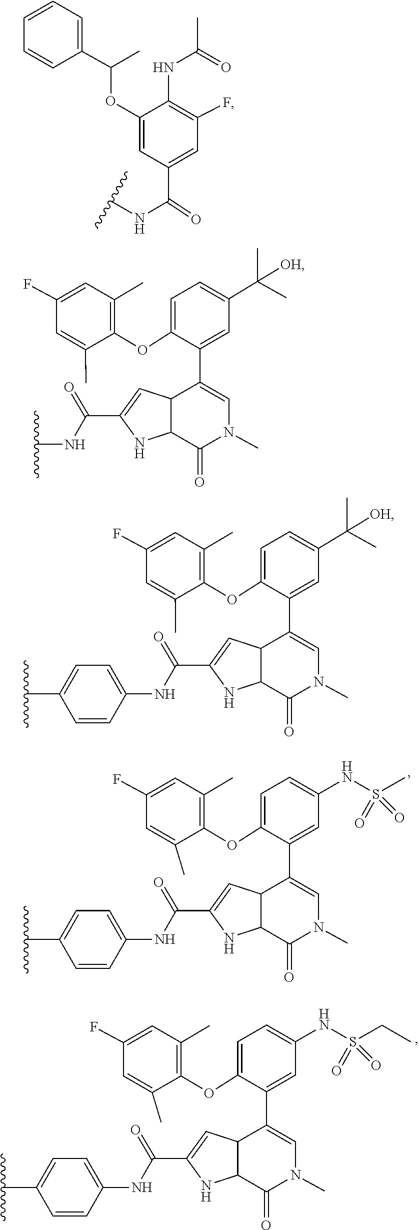

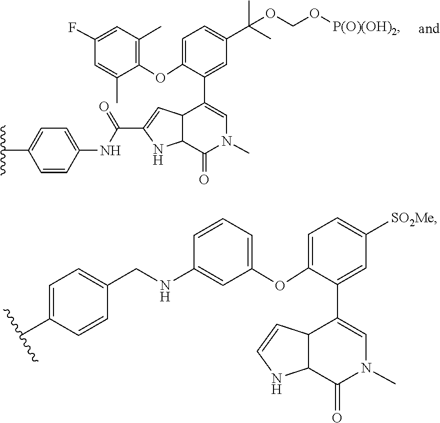

or a pharmaceutically acceptable salt thereof.

In some embodiments, the second terminus is selected from:

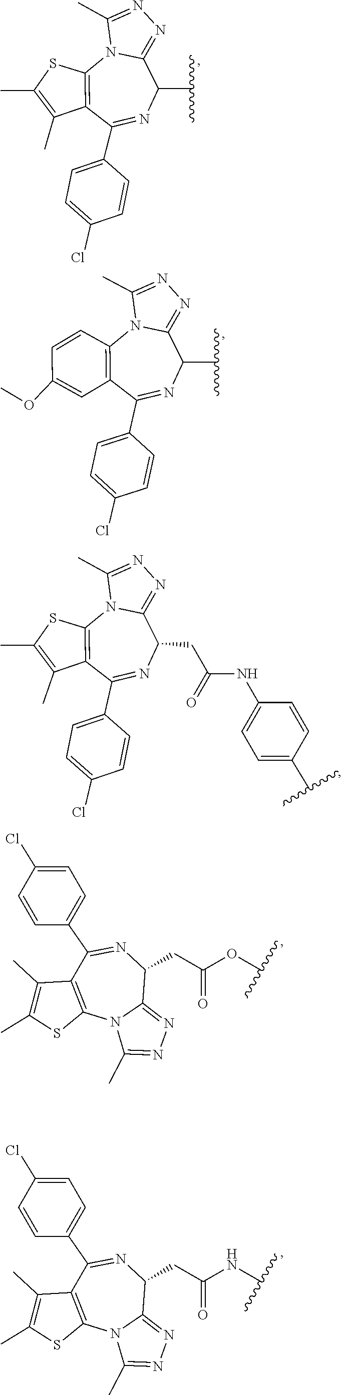

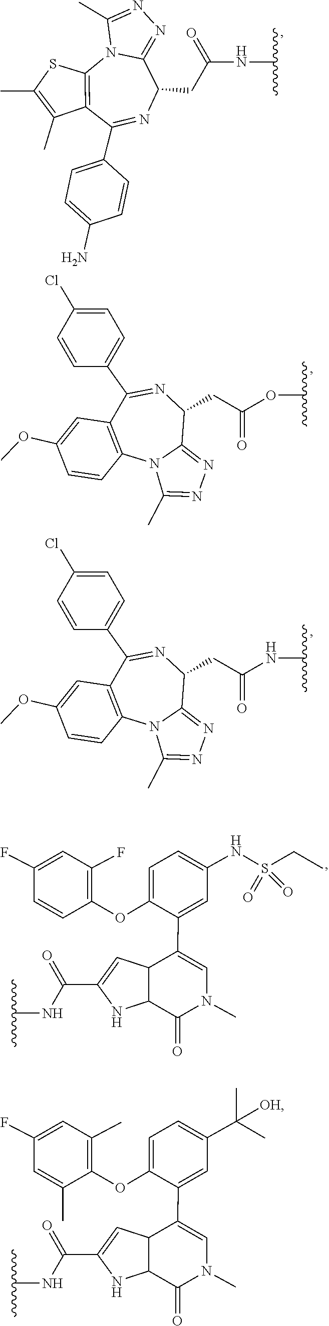

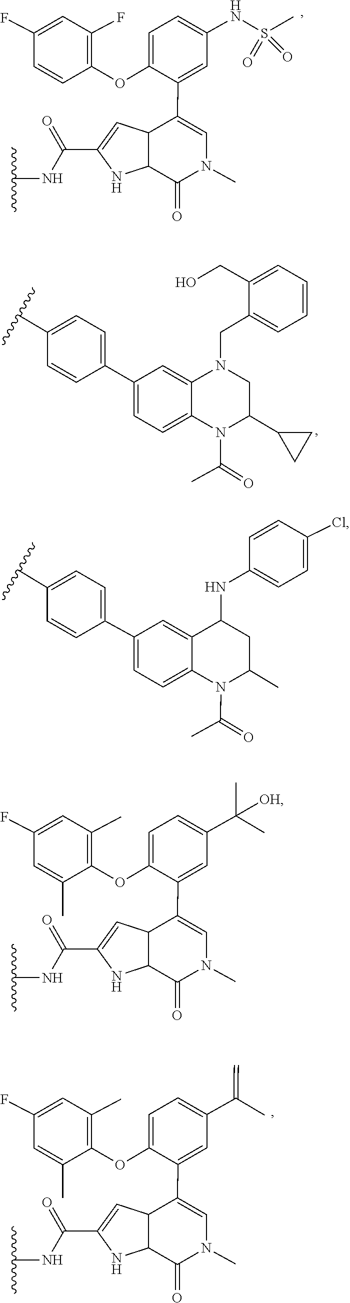 [SMILES: *NC(=O)C1=CC2C(c3cc(NS(C)(=O)=O)ccc3Oc3ccc(F)cc3F)=CN(C)C(=O)C2N1 ; *c1ccc(-c2ccc3c(c2)N(Cc2ccccc2CO)CC(C2CC2)N3C(C)=O)cc1 ; *c1ccc(-c2ccc3c(c2)C(Nc2ccc(Cl)cc2)CC(C)N3C(C)=O)cc1 ; *NC(=O)C1=CC2C(c3cc(C(C)(C)O)ccc3Oc3c(C)cc(F)cc3C)=CN(C)C(=O)C2N1 ; *NC(=O)C1=CC2C(c3cc(C(=C)C)ccc3Oc3c(C)cc(F)cc3C)=CN(C)C(=O)C2N1]

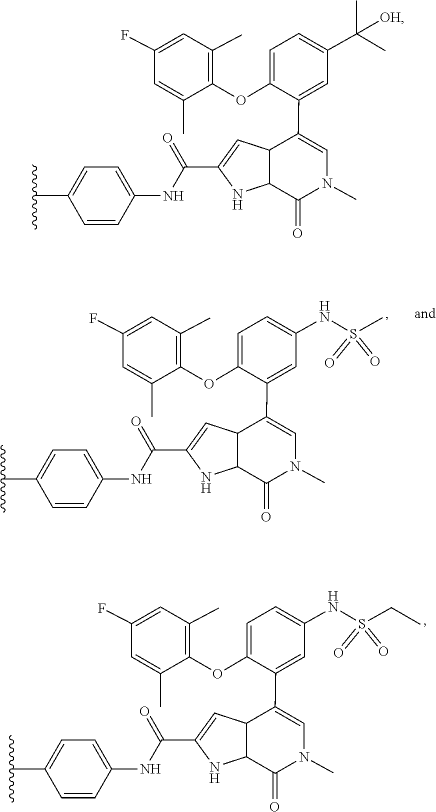 [SMILES: *c1ccc(NC(=O)C2=CC3C(c4cc(C(C)(C)O)ccc4Oc4c(C)cc(F)cc4C)=CN(C)C(=O)C3N2)cc1 ; *c1ccc(NC(=O)C2=CC3C(c4cc(NS(C)(=O)=O)ccc4Oc4c(C)cc(F)cc4C)=CN(C)C(=O)C3N2)cc1 ; *c1ccc(NC(=O)C2=CC3C(c4cc(NS(=O)(=O)CC)ccc4Oc4c(C)cc(F)cc4C)=CN(C)C(=O)C3N2)cc1]

or a pharmaceutically acceptable salt thereof.

In some embodiments, the protein binding moiety is

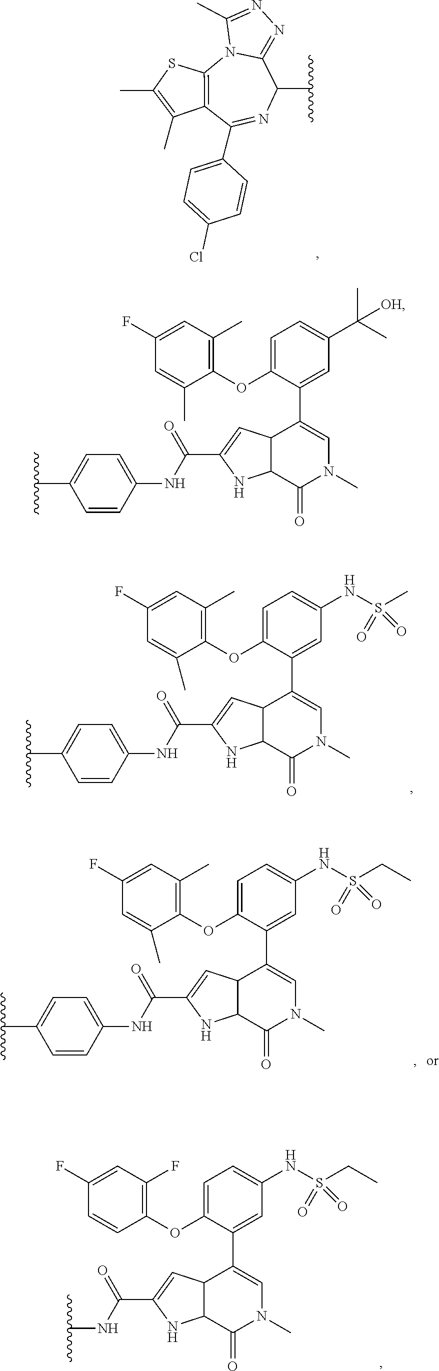 [SMILES: *C1N=C(c2ccc(Cl)cc2)c2c(sc(C)c2C)-n2c(C)nnc21 ; *c1ccc(NC(=O)C2=CC3C(c4cc(C(C)(C)O)ccc4Oc4c(C)cc(F)cc4C)=CN(C)C(=O)C3N2)cc1 ; *c1ccc(NC(=O)C2=CC3C(c4cc(NS(C)(=O)=O)ccc4Oc4c(C)cc(F)cc4C)=CN(C)C(=O)C3N2)cc1 ; *c1ccc(NC(=O)C2=CC3C(c4cc(NS(=O)(=O)CC)ccc4Oc4c(C)cc(F)cc4C)=CN(C)C(=O)C3N2)cc1 ; *NC(=O)C1=CC2C(c3cc(NS(=O)(=O)CC)ccc3Oc3ccc(F)cc3F)=CN(C)C(=O)C2N1]

or a pharmaceutically acceptable salt thereof.

### Linker

The oligomeric backbone contains a linker that connects the first terminus and the second terminus and brings the regulatory molecule in proximity to the target gene to modulate gene expression.

The length of the linker depends on the type of regulatory protein and also the target gene. In some embodiments, the linker has a length of less than about 50 Angstroms. In some embodiments, the linker has a length of about 20 to 30 Angstroms.

In some embodiments, the linker comprises between 5 and 50 chain atoms.

In some embodiments, the linker comprises a multimer having 2 to 50 spacing moieties, wherein the spacing moiety is independently selected from the group consisting of —((CR^{3a}R^{3b})—)_{y}—, —((CR^{3a}R^{3b})ₓ—NR^{4a})_{y}—, —((CR^{3a}R^{3b})ₓ—CH═CH—(CR^{3a}R^{3b})ₓ—O)_{y}—, optionally substituted —C₁₋₁₂ alkyl, optionally substituted C₂₋₁₀ alkenyl, optionally substituted C₂₋₁₀ alkynyl, optionally substituted C₆₋₁₀ arylene, optionally substituted C₃₋₇ cycloalkylene, optionally substituted 5-to 10-membered heteroarylene, optionally substituted 4- to 10-membered heterocycloalkylene, amino acid residue, —O—, —C(O)NR^{4a}—, —NR^{4a}C(O)—, —C(O)—, —NR¹—, —C(O)O—, —O—, —S—, —S(O)—, —SO₂—, —SO₂NR^{4a}—, —NR^{4a}SO₂—, and —P(O)OH—, and any combinations thereof; wherein
each x is independently 2-4;
each y is independently 1-10;
each R^{3a} and R^{3b} are independently selected from hydrogen, optionally substituted alkyl, optionally substituted alkenyl, optionally substituted alkynyl, optionally substituted alkoxy, optionally substituted amino, carboxyl, carboxyl ester, acyl, acyloxy, acyl amino, amino acyl, optionally substituted alkylamide, sulfonyl, optionally substituted thioalkoxy, optionally substituted aryl, optionally substituted heteroaryl, optionally substituted cycloalkyl, and optionally substituted heterocyclyl; and
each R^{4a} is independently a hydrogen or an optionally substituted C₁₋₆ alkyl.

In some embodiments, the oligomeric backbone comprises -(T¹-V₁)ₐ-(T²-V²)_{b}-(T³-V³)_{c}-(T⁴-V⁴)_{d}-(T⁵-V⁵)ₑ—,
wherein a, b, c, d and e are each independently 0 or 1, and where the sum of a, b, c, d and e is 1 to 5;
T¹, T², T³, T⁴ and T⁵ are each independently selected from an optionally substituted (C₁-C₁₂)alkylene, optionally substituted alkenylene, optionally substituted alkynylene, (EA)_{w}, (EDA)ₘ, (PEG)ₙ, (modified PEG)ₙ, (AA)ₚ, —(CR^{2a}OH)ₕ—, optionally substituted (C₆-C₁₀) arylene, optionally substituted C₃₋₇ cycloalkylene, optionally substituted 5- to 10 membered heteroarylene, optionally substituted 4- to 10-membered heterocycloalkylene, an acetal group, a disulfide, a hydrazine, a carbohydrate, a beta-lactam, and an ester,
w is an integer from 1 to 20;
m is an integer from 1 to 20;
n is an integer from 1 to 30;
p is an integer from 1 to 20;
h is an integer from 1 to 12;

EA has the following structure

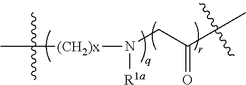

EDA has the following structure:

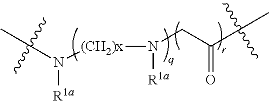

wherein each q is independently an integer from 1 to 6, each x is independently an integer from 1 to 4, and each r is independently 0 or 1;
(PEG)ₙ has the structure of —(CR^{2a}R^{2b}—CR^{2a}R^{2b}—O)ₙ—CR^{2a}R^{2b}—; —
(modified PEG)n has the structure of replacing at least one —(CR^{2a}R^{2b}—CR^{2a}R^{2b}—O)— in (PEG)n with —(CH₂—CR^{2a} ═CR^{2a}—CH₂—O)— or —(CR^{2a}R^{2b}—CR^{2a}R^{2b}—S)_;
AA is an amino acid residue;
V¹, V², V³, V⁴ and V⁵ are each independently selected from the group consisting of a bond, CO—, —NR^{1a}—, —CONR^{1a}—, —NR^{1a}CO—, —CONR^{1a}C₁₋₄ alkyl-, —NR^{1a}CO—C₁₋₄ alkyl-, —C(O)O—, —OC(O)—, —O—, —S—, —S(O)—, —SO₂—, —SO₂NR^{1a}—, —NR^{1a}SO₂— and —P(O)OH—;
each R^{1a} is independently hydrogen or and optionally substituted C₁₋₆ alkyl; and
each R^{2a} and R^{2b} are independently selected from hydrogen, alkyl, substituted alkyl, alkenyl, substituted alkenyl, alkynyl, substituted alkynyl, halogen, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl.

In some embodiments, the a, b, c, d and e are each independently 0 or 1, where the sum of a, b, c, d and e is 1. In some embodiments, the a, b, c, d and e are each independently 0 or 1, where the sum of a, b, c, d and e is 2. In some embodiments, the a, b, c, d and e are each independently 0 or 1, where the sum of a, b, c, d and e is 3. In some embodiments, the a, b, c, d and e are each independently 0 or 1, where the sum of a, b, c, d and e is 4. In some embodiments, the a, b, c, d and e are each independently 0 or 1, where the sum of a, b, c, d and e is 5.

In some embodiments, n is 3-9. In some embodiments, n is 4-8. In some embodiments, n is 5 or 6.

In some embodiments, T¹, T², T³, and T⁴, and T⁵ are each independently selected from (C₁-C₁₂)alkyl, substituted (C₁-C₁₂)alkyl, (EA)_{w}, (EDA)ₘ, (PEG)ₙ, (modified PEG)n, (AA)ₚ, —(CR^{2a}OH)ₕ—, phenyl, substituted phenyl, piperidin-4-amino (P4A), para-amino-benzyloxycarbonyl (PABC), meta-amino-benzyloxycarbonyl (MABC), para-amino-benzyloxy (PABO), meta-amino-benzyloxy (MABO), para-aminobenzyl, an acetal group, a disulfide, a hydrazine, a carbohydrate, a beta-lactam, an ester, (AA)ₚ-MABC-(AA)ₚ, (AA)ₚ-MABO-(AA)ₚ, (AA)ₚ-PABO-(AA)ₚ and (AA)ₚ-PABC-(AA)ₚ, In some embodiments, piperidin-4-amino (P4A) is

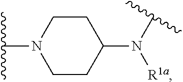

wherein R^{1a} is H or C₁₋₆ alkyl.

In some embodiments, T¹, T², T³, T⁴ and T⁵ are each independently selected from (C₁-C₁₂)alkyl, substituted (C₁-C₁₂)alkyl, (EA)_{w}, (EDA)ₘ, (PEG)ₙ, (modified PEG)ₙ, (AA)ₚ, —(CR^{2a}OH)ₕ—, optionally substituted (C₆-C₁₀) arylene, 4-10 membered heterocycloalkene, optionally substituted 5-10 membered heteroarylene. In some embodiments, EA has the following structure:

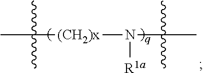

and

EDA has the following structure:

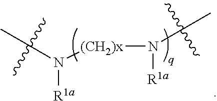

In some embodiments, x is 2-3 and q is 1-3 for EA and EDA. In some embodiments, R^{1a} is H or C₁₋₆ alkyl.

In some embodiments, T⁴ or T⁵ is an optionally substituted (C₆-C₁₀) arylene.

In some embodiments, T⁴ or T⁵ is phenylene or substituted phenylene. In some embodiments, T⁴ or T⁵ is phenylene or phenylene substituted with 1-3 substituents selected from —C₁₋₆ alkyl, halogen, OH or amine. In some embodiments, T⁴ or T⁵ is 5-10 membered heteroarylene or substituted heteroarylene. In some embodiments, T⁴ or T⁵ is 4-10 membered heterocylene or substituted heterocylene. In some embodiments, T⁴ or T⁵ is heteroarylene or heterocylene optionally substituted with 1-3 substituents selected from —C₁₋₆ alkyl, halogen, OH or amine.

In some embodiments, T¹, T², T³, T⁴ and T⁵ and V¹, V², V³, V⁴ and V⁵ are selected from the following Table 2.

TABLE 2
Representative linkers.

| T¹ | V¹ | T² | V² | T³ | V³ | T⁴ | V⁴ | T⁵ | V⁵ |
|---|---|---|---|---|---|---|---|---|---|
| (C₁-C₁₂) | CONR^{1a} | (EA)_{w} | CO | (PEG)ₙ | NR¹¹CO | — | — | — | — |
| alkylene | | | | | | | | | |
| (C₁-C₁₂) | CONR^{1a} | (EA)_{w} | CO | (PEG)ₙ | O | arylene | NR¹¹CO | — | — |
| alkylene | | | | | | | | | |
| (C₁-C₁₂) | CONR^{1a} | (EA)_{w} | CO | (PEG)ₙ | O | Subst. | NR¹¹CO | — | — |
| alkylene | | | | | | arylene | | | |
| (C₁-C₁₂) | CONR^{1a} | (EA)_{w} | CO | (PEG)ₙ | O | NR¹¹CO | (C₁-C₁₂) | Subst. | NR¹¹CO |
| alkylene | | | | | | | alkyl | arylene | |
| (C₁-C₁₂) | CONR^{1a} | (EA)_{w} | CO | (C₁-C₁₂) | NR¹¹CO- | Subst. | NR¹¹ | — | — |
| alkylene | | | | alkyl | C₁₋₄ alkyl | arylene | | | |
| (C₁-C₁₂) | CONR^{1a} | (EA)_{w} | CO | (PEG)ₙ | O | Subst. | — | — | — |
| alkylene | | | | | | arylene | | | |
| (PEG)ₙ | CONR^{1a-} | — | — | — | — | — | — | — | — |
| | C₁₋₄ alkyl | | | | | | | | |
| (EA)_{w} | CO | (C₁-C₁₂) | CONR¹¹⁻ | — | — | — | — | — | — |
| | | alky1 | C₁₋₄ alkyl | | | | | | |
| (C₁-C₁₂) | CONR^{1a} | (EA)_{w} | CO | (PEG)ₙ | NR¹¹CO- | — | — | — | — |
| alkylene | | | | | C₁₋₄ alkyl | | | | |
| (EA)_{w} | CO | (PEG)ₙ | O | phenyl | NR¹¹CO- | — | — | — | — |
| | | | | | C₁₋₄ alkyl | | | | |
| (C₁-C₁₂) | CONR^{1a} | (PEG)ₙ | CO | — | — | — | — | — | — |
| alkylene | | | | | | | | | |
| (C₁-C₁₂) | CONR^{1a} | (EA)_{w} | CO | modifd. | O | arylene | NR¹¹CO | — | — |
| alkylene | | | | (PEG)ₙ | | | | | |

In some embodiments, the linker comprises

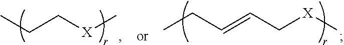

or any combinations thereof, wherein r is an integer between 1 and 10, preferably between 3 and 7; and X is O, S, or NR^{1a}. In some embodiments, X is O or NR^{1a}. In some embodiments, X is O.

In some embodiments, the linker comprise a

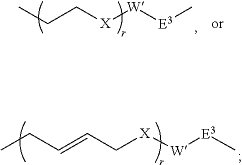

or any combinations thereof; wherein at least one —(CH₂—CH₂—O)— is replaced with —((CR^{1a}R^{1b})ₓ—CH═CH—(CR^{1a}R^{1b})ₓ—O)—, or any combinations thereof; W′ is absent, (CH₂)₁₋₅, —(CH₂)₁₋₅O, (CH₂)₁₋₅-C(O)NH—(CH₂)is-O, (CH₂)₁₋₅-C(O)NH—(CH₂)₁₋₅, —(CH₂)₁₋₅NHC(O)—(CH₂)is-O, or —(CH₂)₁₋₅—NHC(O)—(CH₂)₁₋₅—; E³ is an optionally substituted C₆₋₁₀ arylene group, optionally substituted 4-10 membered heterocycloalkylene, or optionally substituted 5-10 membered heteroarylene; X is O, S, or NH; each R^{1a} and R^{1b} are independently H or C₁₋₆ alkyl; r is an integer between 1 and 10; and x is an integer between 1 and 15. In some embodiments, X is 0. In some embodiments, X is NH. In some embodiments, E³ is a C₆₋₁₀ arylene group optionally substituted with 1-3 substituents selected from —C₁₋₆ alkyl, halogen, OH or amine.

In some embodiments, E³ is a phenylene or substituted phenylene.

In some embodiments, the linker comprise a

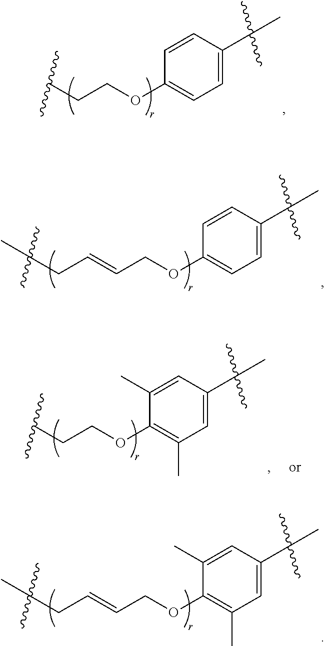

In some embodiments, the linker comprises —X(CH₂)ₘ(CH₂CH₂O)ₙ—, wherein X is —O—, —NH—, or —S—, wherein m is 0 or greater and n is at least 1.

In some embodiments, the linker comprises

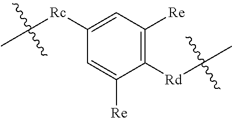

following the second terminus, wherein Rₑ is selected from a bond, —N(R^{1a})—, —O—, and —S—; R_{d} is selected from —N(R^{1a})—, - and —S—; and Rₑ is independently selected from hydrogen and optionally substituted C₁₋₆ alkyl.

In some embodiments, the linker comprises one or more structures selected from

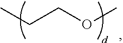

—C₁₋₁₂ alkyl, arylene, cycloalkylene, heteroarylene, heterocycloalkylene, —O—, —C(O)NR^{1a}—, —C(O)—, —NR^{1a}—, —(CH₂CH₂CH₂O)_{y}—, and —(CH₂CH₂CH₂NR^{1a})_{y}—, wherein each d and y are independently 1-10, and each R^{1a} is independently hydrogen or C₁₋₆ alkyl. In some embodiments, d is 4-8.

In some embodiments, the linker comprises

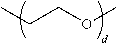

and each d is independently 3-7. In some embodiments, d is 4-6.

In some embodiments, the linker comprises —N(R^{1a})(CH₂)ₓN(R^{1b})(CH₂)ₓN—, wherein R^{1a} and R^{1b}are each independently selected from hydrogen or optionally substituted C₁-C₆ alkyl; and each x is independently an integer in the range of 1-6.

In some embodiments, the linker comprises the linker comprises —(CH₂—C(O)N(R″)—(CH₂)q-N(R′)—(CH₂)_{q}—N(R″)C(O)—(CH₂)ₓ—C(O)N(R″)-A²-, —(CH₂)ₓ—C(O)N(R″)—(CH₂CH₂O)_{y}(CH₂)ₓ—C(O)N(R″)-A²-, —C(O)N(R″)—(CH₂)q-N(R′)—(CH₂)q-N(R″)C(O)—(CH₂)ₓ-A²-, —(CH₂)ₓ—O—(CH₂CH₂O)_{y}—(CH₂)ₓ—N(R″)C(O)—(CH₂)ₓ-A²-, or —N(R″)C(O)—(CH₂)—C(O)N(R″)—(CH₂)ₓ—O(CH₂CH₂O)_{y}(CH₂)ₓ-A²-; wherein R′ is methyl; R″ is hydrogen; each x and y are independently an integer from 1 to 10; each q is independently an integer from 2 to 10; and each A² is independently selected from a bond, an optionally substituted C₁₋₁₂ alkyl, an optionally substituted C₆₋₁₀ arylene, optionally substituted C₃₋₇ cycloalkylene, optionally substituted 5- to 10-membered heteroarylene, and optionally substituted 4- to 10-membered heterocycloalkylene.

In some embodiments, the linker comprises —(CH₂CH₂—O)ₓ₁— or —(CH₂CH₂—O)ₓ₂-A²-(CH₂CH₂—O)ₓ₃—, wherein A² is an optionally substituted 4- to 10-membered heterocycloalkylene or spirocyclene, and each x₁, x2, and x3 is independently an integer from 1-15.

In some embodiments, A² is selected from

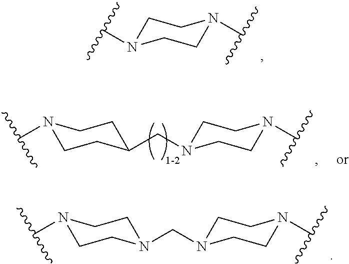

In some embodiments, A² is

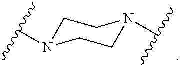 [SMILES: *N1CCN(*)CC1]

In some embodiments, A² is

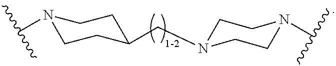

In some embodiments, A² is

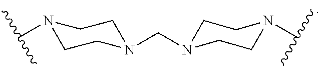 [SMILES: *N1CCN(CN2CCN(*)CC2)CC1]

In some embodiments, A² comprises a moiety having the structure:

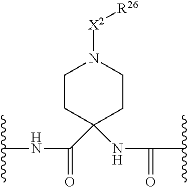

wherein,
X² is absent or —C(O)—; and
R²⁶ is an optionally substituted C₁₋₅₀ alkyl or C₁₋₅₀ heteroalkyl.

In some embodiments, X² is —C(O)—. In some embodiments, X² is absent.

In some embodiments, R²⁶ is C₁₋₅₀ alkyl. In some embodiments, R²⁶ is C₁₋₄₀ alkyl. In some embodiments, R²⁶ is C₁₋₃₀ alkyl. In some embodiments, R²⁶ is C₁₋₂₀ alkyl. In some embodiments, R²⁶ is C₁₋₁₀ alkyl. In some embodiments, R²⁶ is C₁₋₅₀ heteroalkyl. In some embodiments, R²⁶ is C₁₋₄₀ heteroalkyl. In some embodiments, R²⁶ is C₁₋₃₀ heteroalkyl. In some embodiments, R²⁶ is C₁₋₂₀ heteroalkyl. In some embodiments, R²⁶ is C₁₋₁₀ heteroalkyl. In some embodiments, the heteroalkyl is polyethylene glycol (PEG).

In some embodiments, the linker is joined with the first terminus with a group selected from —CO—, —NR^{1a}—, C₁₋₁₂ alkyl, —CONR^{1a}—, and —NR^{1a}CO—; wherein each R^{1a} is independently a hydrogen or optionally substituted C₁₋₆ alkyl or optionally substituted —C₁₋₁₂ alkylene, optionally substituted C₂₋₁₀ alkenylene, optionally substituted C₂₋₁₀ alkynylene, optionally substituted C₆₋₁₀ arylene, optionally substituted C₃₋₇ cycloalkylene, optionally substituted 5- to 10-membered heteroarylene, and optionally substituted 4- to 10-membered heterocycloalkylene.

In some embodiments, the linker is joined with the first terminus with a group selected from —CO—, —NR^{1a}—, —CONR^{1a}—, —NR^{1a}CO—, —CONR^{1a}C₁₋₄alkyl-, —NR^{1a}CO—C₁₋₄alkyl-, —C(O)O—, —OC(O)—, —O—, —S—, —S(O)—, —SO₂—, —SO₂NR^{1a}—, —NR¹SO₂—, —P(O)OH—, —((CH₂)ₓ—O)—, —((CH₂)_{y}—NR^{1a})—, optionally substituted —C₁₋₁₂ alkylene, optionally substituted C₂₋₁₀ alkenylene, optionally substituted C₂₋₁₀ alkynylene, optionally substituted C₆₋₁₀ arylene, optionally substituted C₃₋₇ cycloalkylene, optionally substituted 5- to 10-membered heteroarylene, and optionally substituted 4- to 10-membered heterocycloalkylene, wherein each x is independently 1-4, each y is independently 1-4, and each R^{1a} is independently a hydrogen or optionally substituted C₁₋₆ alkyl.

In some embodiments, the linker is joined with the first terminus with a group selected from —CO—, —NR^{1a} —, C₁₋₁₂ alkyl, —CONR^{1a} —, and —NR^{1a}CO—.

In some embodiments, the linker is joined with the second terminus with a group selected from —CO—, —NR^{1a}—, —CONR^{1a}—, —NR^{1a}CO—, —CONR^{1a}C₁₋₄alkyl-, —NR^{1a}CO—C₁₋₄alkyl-, —C(O)O—, —OC(O)—, —O—, —S—, —S(O)—, —SO₂—, —SO₂NR^{1a}—, —NR¹SO₂—, —P(O)OH—, —((CH₂)ₓ—O)—, —((CH₂)_{y}—NR^{1a})—, optionally substituted —C₁₋₁₂ alkylene, optionally substituted C₂₋₁₀ alkenylene, optionally substituted C₂₋₁₀ alkynylene, optionally substituted C₆₋₁₀ arylene, optionally substituted C₃₋₇ cycloalkylene, optionally substituted 5- to 10-membered heteroarylene, and optionally substituted 4- to 10-membered heterocycloalkylene, wherein each x is independently 1-4, each y is independently 1-4, and each R^{1a} is independently a hydrogen or optionally substituted C₁₋₆ alkyl.

In some embodiments, the linker is joined with the second terminus with a group selected from —CO—, —NR^{1a}—, —CONR^{1a}—, —NR^{1a}CO—, —((CH₂)ₓ—O)—, —((CH₂)_{y}—NR^{1a})—, —O—, optionally substituted —C₁₋₁₂ alkyl, optionally substituted C₆₋₁₀ arylene, optionally substituted C₃₋₇ cycloalkylene, optionally substituted 5- to 10-membered heteroarylene, and optionally substituted 4- to 10-membered heterocycloalkylene, wherein each x is independently 1-4, each y is independently 1-4, and each R¹ is independently a hydrogen or optionally substituted C₁₋₆ alkyl. In some embodiments, the linker is joined with the second terminus with a group selected from —CO—, —NR^{1a}—, C₁₋₁₂ alkyl, —CONR^{1a} —, and —NR^{1a}CO—.

In some embodiments, the linker is joined with the first or the second terminus with a group selected from optionally substituted 4- to 10-membered heterocycloalkylene. In some embodiments, the linker is joined with the second terminus with a group selected from optionally substituted 4- to 10-membered heterocycloalkylene.

In some embodiments, the linker is joined with the second terminus with a moiety comprising a structure of Formula (C-1), or a pharmaceutically acceptable salt thereof:

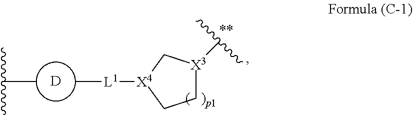

wherein,
Ring D is absent or an arylene or heterocycloaklylene;
L¹ is absent or an optionally substituted alkylene or alkenelene;
each X³ and X⁴ is independently CH or N;
p₁ is 0-3; and
* denotes attachment to the second terminus.

In some embodiments, Ring D is absent. In some embodiments, Ring D is C₄-C₇ heterocycloaklylene.

In some embodiments, X³ is N. In some embodiments, X³ is CH.

In some embodiments, X⁴ is N. In some embodiments, X⁴ is CH.

In some embodiments, the linker is joined with the second terminus with a moiety comprising a structure of Formula (C-2), or a pharmaceutically acceptable salt thereof:

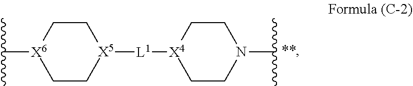

wherein,
each X⁵ and X⁶ is independently N or CH.

In some embodiments, each of X⁴ and X⁵ is independently N or CH; and X⁶ is N.

In some embodiments, L¹ is absent.

In some embodiments, L¹ is —(C^{R1G}R^{1G})ₓ-(alkylene)₂-(C^{R1G}R^{1G})_{y}—; wherein x and y are each independently 0 or 1; and each R^{1G} is hydrogen or C₁-C₃ alkyl.

In some embodiments, L¹ is C₁-C₃ alkylene or C₁-C₃ alkenelene.

In some embodiments, L¹ is —CH₂—, —CH₂CH₂—, —C≡C—, or —C≡C—C≡C— In some embodiments, L¹ is —CH₂— or —CH₂CH₂—. In some embodiments, L¹ is —C≡C—. In some embodiments, L¹ is —C≡C—C═C—.

In some embodiments, the linker is joined with the second terminus with a moiety comprising a structure of Formula (C-3), or a pharmaceutically acceptable salt thereof:

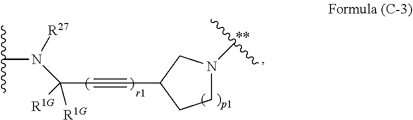

wherein,
p₁ is 0-3;
r₁ is 1-3;
R²⁷ is an optionally substituted C₁₋₅₀ alkyl, C₁₋₅₀ heteroalkyl, —C(O)(C₁₋₅₀ alkyl), or —C(O)(C₁₋₅₀ heteroalkyl), wherein each alkyl and heteroalkyl is optionally substituted;
each R^{1G} is independently hydrogen or C₁-C₃ alkyl; and
** denotes attachment to the second terminus.

In some embodiments, R²⁷ is an optionally substituted C₁₋₅₀ alkyl or C₁₋₅₀ heteroalkyl. In some embodiments, R²⁷ is —C(O)(C₁₋₅₀ alkyl) or —C(O)(C₁₋₅₀ heteroalkyl), wherein each alkyl and heteroalkyl is optionally substituted.

some embodiments, R²⁷ is C₁₋₅₀ alkyl. In some embodiments, R²⁷ is C₁₋₄₀ alkyl. In some embodiments, R²⁷ is C₁₋₃₀ alkyl. In some embodiments, R²⁷ is C₁₋₂₀ alkyl. In some embodiments, R²⁷ is C₁₋₁₀ alkyl. In some embodiments, R²⁷ is C₁₋₅₀ heteroalkyl. In some embodiments, R²⁷ is C₁₋₄₀ heteroalkyl. In some embodiments, R²⁷ is C₁₋₃₀ heteroalkyl. In some embodiments, R²⁷ is C₁₋₂₀ heteroalkyl. In some embodiments, R²⁷ is C₁₋₁₀ heteroalkyl. In some embodiments, the heteroalkyl is polyethylene glycol (PEG).

In some embodiments, each R^{1G} is independently hydrogen. In some embodiments, R^{1G} is independently C₁-C₃ alkyl. In some embodiments, the C₁-C₃ alkyl is methyl, ethyl or propyl. In some embodiments, each R^{1G} is independently methyl.

In some embodiments, p₁ is 0, 1, or 2. In some embodiments, p₁ is 0. In some embodiments, p₁ is 1. In some embodiments, p₁ is 2.

In some embodiments, r₁ is 1 or 2. In some embodiments, r₁ is 1. In some embodiments, r₁ is 2.

In some embodiments, the linker is joined with the first and/or the second terminus with a group selected from:

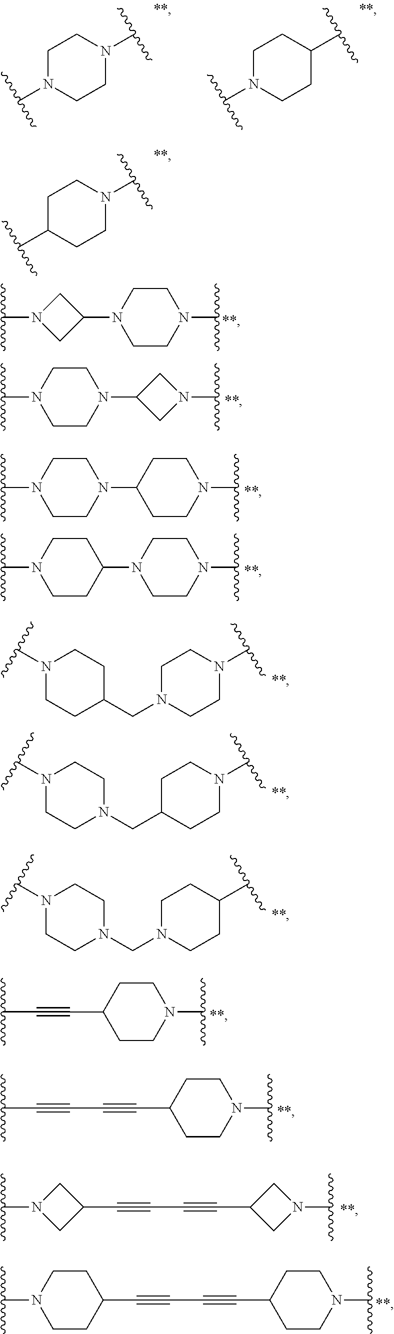

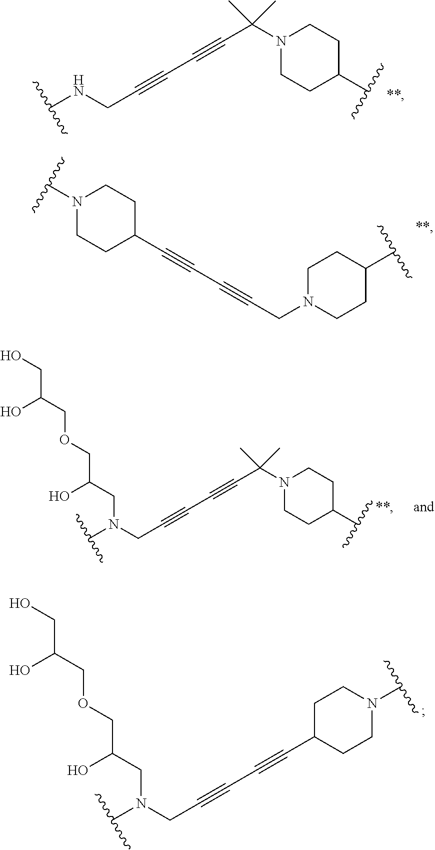 [SMILES: *NCC#CC#CC(C)(C)N1CCC(*)CC1 ; *C1CCN(CC#CC#CC2CCN(*)CC2)CC1 ; *C1CCN(C(C)(C)C#CC#CCN(*)CC(O)COCC(O)CO)CC1 ; *N1CCC(C#CC#CCN(*)CC(O)COCC(O)CO)CC1]

wherein ** denotes the connection to the first and/or the second terminus.

In some embodiments, the linker is joined with the first and/or the second terminus with a group selected from:

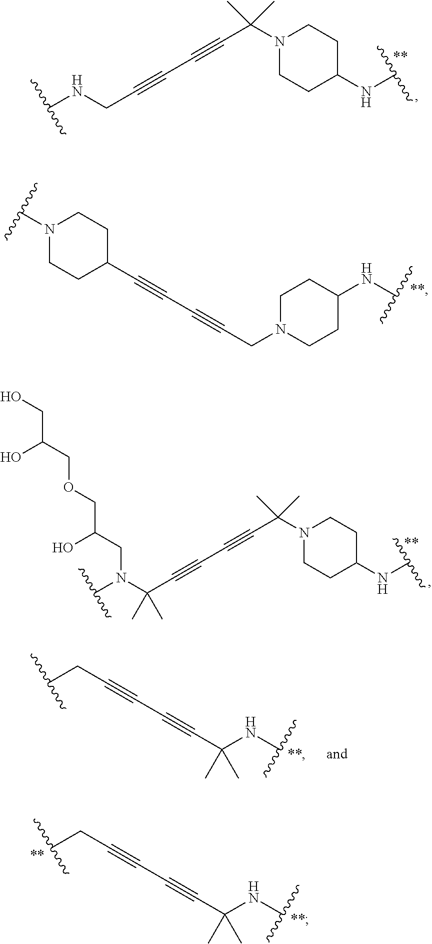 [SMILES: *NCC#CC#CC(C)(C)N1CCC(N*)CC1 ; *NC1CCN(CC#CC#CC2CCN(*)CC2)CC1 ; *NC1CCN(C(C)(C)C#CC#CC(C)(C)N(*)CC(O)COCC(O)CO)CC1 ; *CC#CC#CC(C)(C)N* ; *CC#CC#CC(C)(C)N*]

wherein ** denotes the connection to the first and/or the second terminus

In some embodiments, the linker is independently joined with the first and the second terminus with one of the groups described above. In some embodiments, the linker is joined to the first terminus with any of the groups described above. In some embodiments, the linker is joined to the second terminus with any of the groups described above.

### Cell-Penetrating Ligand

In certain embodiments, the compounds comprise a cell-penetrating ligand moiety.

In certain embodiments, the cell-penetrating ligand moiety is a polypeptide.

In certain embodiments, the cell-penetrating ligand moiety is a polypeptide containing fewer than 30 amino acid residues.

In certain embodiments, the polypeptide is chosen from any one of SEQ ID NO. 1 to SEQ ID NO. 37, inclusive.

Also provided are embodiments wherein any embodiment above may be combined with any one or more of these embodiments, provided the combination is not mutually exclusive.

As used herein, two embodiments are “mutually exclusive” when one is defined to be something which is different than the other. For example, an embodiment wherein two groups combine to form a cycloalkyl is mutually exclusive with an embodiment in which one group is ethyl the other group is hydrogen. Similarly, an embodiment wherein one group is CH₂ is mutually exclusive with an embodiment wherein the same group is NH.

In some embodiments, non-limiting examples of the transcription modulator compounds described herein are presented in Table 3, or a pharmaceutically acceptable salt thereof.

Table 3. Compounds of the disclosure.

TABLE 3

| Compounds of the disclosure. | |
|---|---|
| 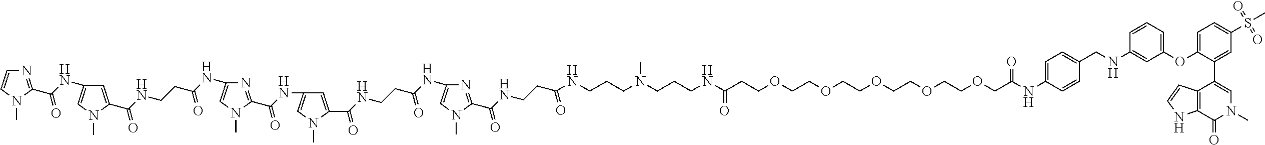 [SMILES: CN(CCCNC(=O)CCNC(=O)c1nc(NC(=O)CCNC(=O)c2cc(NC(=O)c3nc(NC(=O)CCNC(=O)c4cc(NC(=O)c5nccn5C)cn4C)cn3C)cn2C)cn1C)CCCNC(=O)CCOCCOCCOCCOCCOCC(=O)Nc1ccc(CNc2cccc(Oc3ccc(S(C)(=O)=O)cc3-c3cn(C)c(=O)c4[nH]ccc34)c2)cc1] | 1 |
| 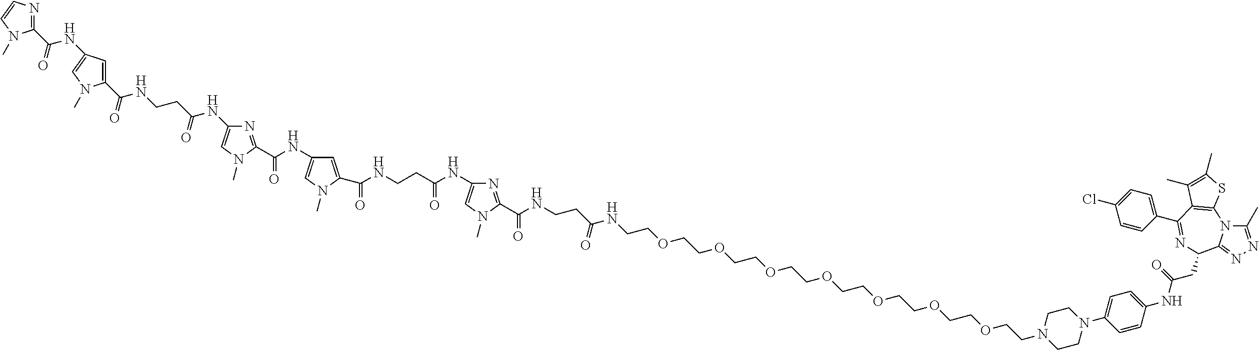 [SMILES: Cc1sc2c(c1C)C(c1ccc(Cl)cc1)=N[C@@H](CC(=O)Nc1ccc(N3CCN(CCOCCOCCOCCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)CC3)cc1)c1nnc(C)n1-2] | 2 |
| 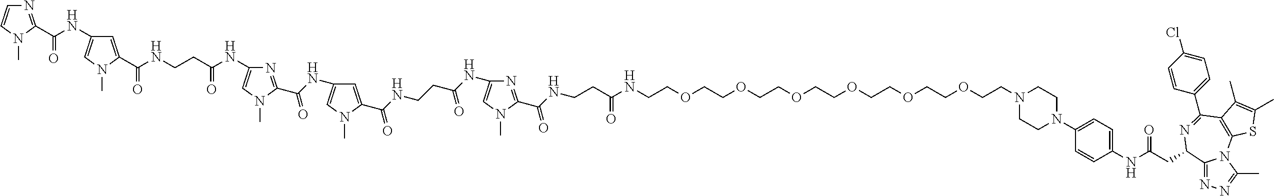 [SMILES: Cc1sc2c(c1C)C(c1ccc(Cl)cc1)=N[C@@H](CC(=O)Nc1ccc(N3CCN(CCOCCOCCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)CC3)cc1)c1nnc(C)n1-2] | 3 |
| 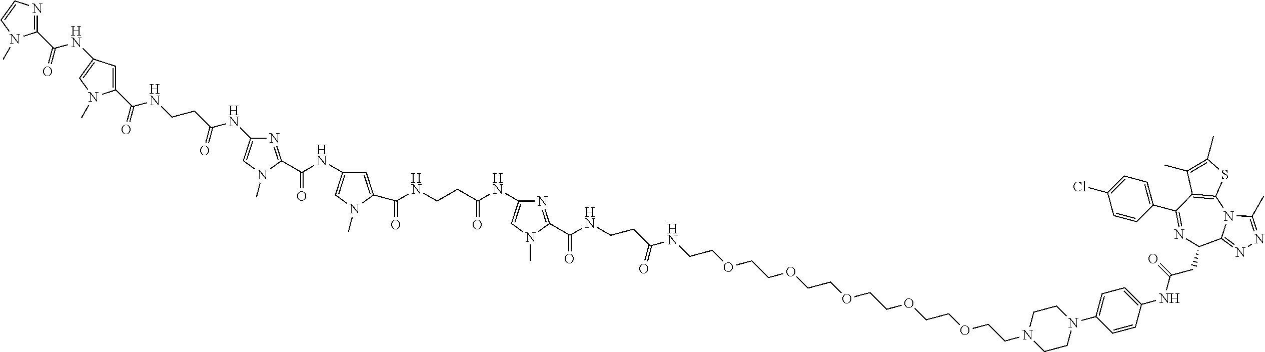 [SMILES: Cc1sc2c(c1C)C(c1ccc(Cl)cc1)=N[C@@H](CC(=O)Nc1ccc(N3CCN(CCOCCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)CC3)cc1)c1nnc(C)n1-2] | 4 |
| 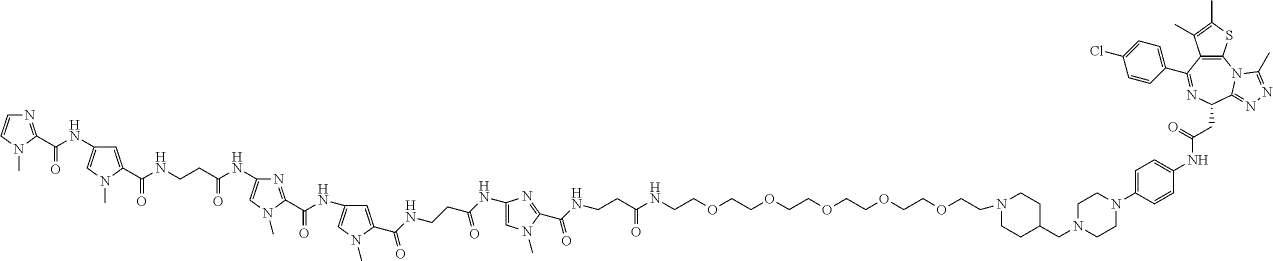 [SMILES: Cc1sc2c(c1C)C(c1ccc(Cl)cc1)=N[C@@H](CC(=O)Nc1ccc(N3CCN(CC4CCN(CCOCCOCCOCCOCCOCCNC(=O)CCNC(=O)c5nc(NC(=O)CCNC(=O)c6cc(NC(=O)c7nc(NC(=O)CCNC(=O)c8cc(NC(=O)c9nccn9C)cn8C)cn7C)cn6C)cn5C)CC4)CC3)cc1)c1nnc(C)n1-2] | 5 |
| 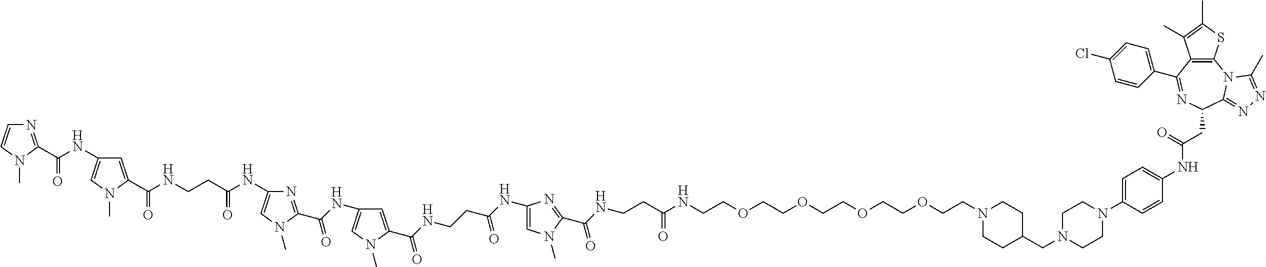 [SMILES: Cc1sc2c(c1C)C(c1ccc(Cl)cc1)=N[C@@H](CC(=O)Nc1ccc(N3CCN(CC4CCN(CCOCCOCCOCCOCCNC(=O)CCNC(=O)c5nc(NC(=O)CCNC(=O)c6cc(NC(=O)c7nc(NC(=O)CCNC(=O)c8cc(NC(=O)c9nccn9C)cn8C)cn7C)cn6C)cn5C)CC4)CC3)cc1)c1nnc(C)n1-2] | 6 |
| 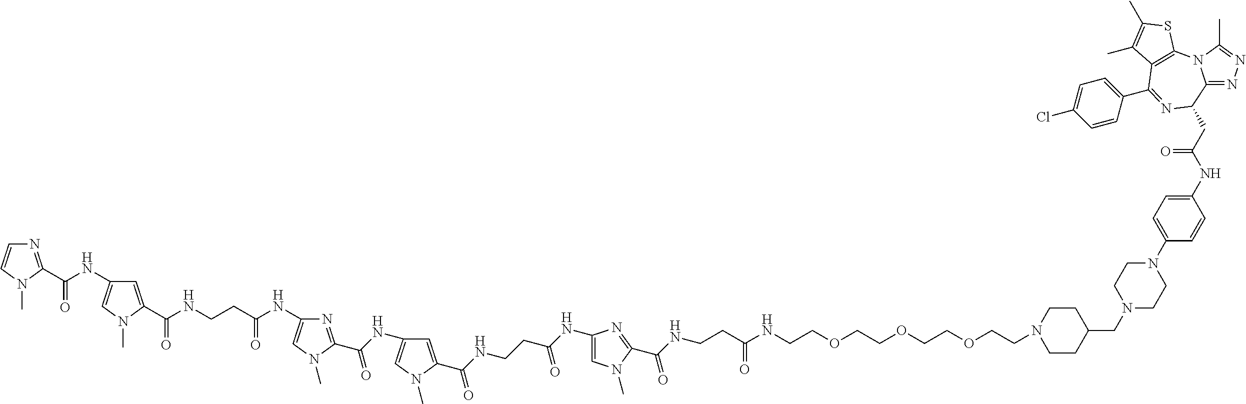 [SMILES: Cc1sc2c(c1C)C(c1ccc(Cl)cc1)=N[C@@H](CC(=O)Nc1ccc(N3CCN(CC4CCN(CCOCCOCCOCCNC(=O)CCNC(=O)c5nc(NC(=O)CCNC(=O)c6cc(NC(=O)c7nc(NC(=O)CCNC(=O)c8cc(NC(=O)c9nccn9C)cn8C)cn7C)cn6C)cn5C)CC4)CC3)cc1)c1nnc(C)n1-2] | 7 |
| 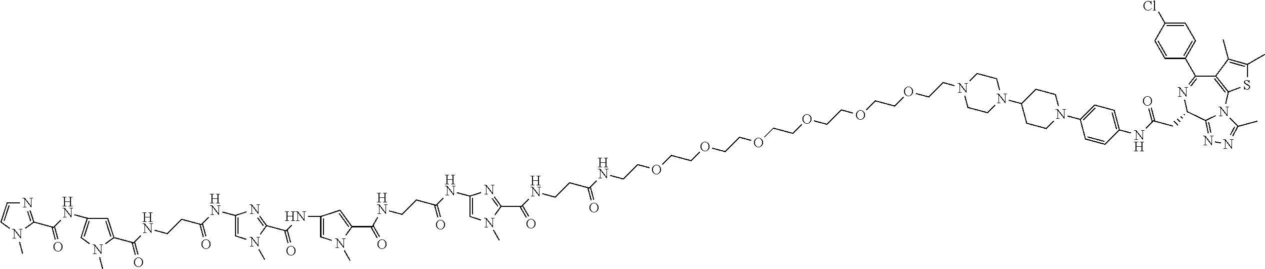 [SMILES: Cc1sc2c(c1C)C(c1ccc(Cl)cc1)=N[C@@H](CC(=O)Nc1ccc(N3CCC(N4CCN(CCOCCOCCOCCOCCOCCOCCNC(=O)CCNC(=O)c5nc(NC(=O)CCNC(=O)c6cc(NC(=O)c7nc(NC(=O)CCNC(=O)c8cc(NC(=O)c9nccn9C)cn8C)cn7C)cn6C)cn5C)CC4)CC3)cc1)c1nnc(C)n1-2] | 8 |
| 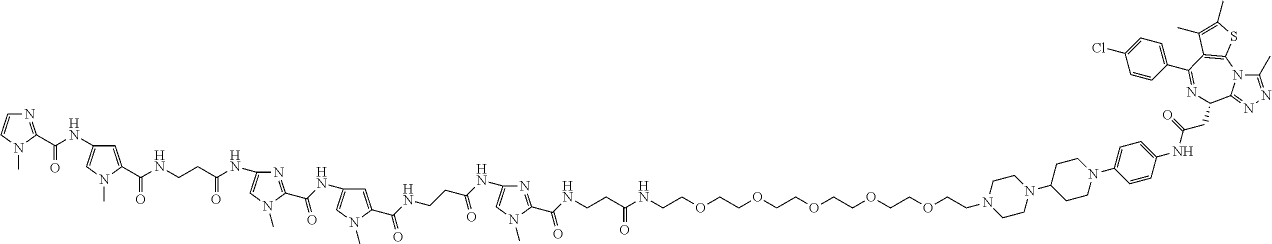 [SMILES: Cc1sc2c(c1C)C(c1ccc(Cl)cc1)=N[C@@H](CC(=O)Nc1ccc(N3CCC(N4CCN(CCOCCOCCOCCOCCOCCNC(=O)CCNC(=O)c5nc(NC(=O)CCNC(=O)c6cc(NC(=O)c7nc(NC(=O)CCNC(=O)c8cc(NC(=O)c9nccn9C)cn8C)cn7C)cn6C)cn5C)CC4)CC3)cc1)c1nnc(C)n1-2] | 9 |
| 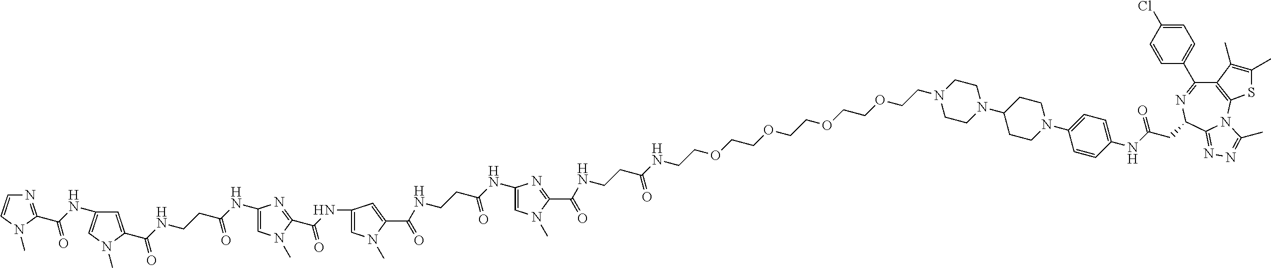 [SMILES: Cc1sc2c(c1C)C(c1ccc(Cl)cc1)=N[C@@H](CC(=O)Nc1ccc(N3CCC(N4CCN(CCOCCOCCOCCOCCNC(=O)CCNC(=O)c5nc(NC(=O)CCNC(=O)c6cc(NC(=O)c7nc(NC(=O)CCNC(=O)c8cc(NC(=O)c9nccn9C)cn8C)cn7C)cn6C)cn5C)CC4)CC3)cc1)c1nnc(C)n1-2] | 10 |
| 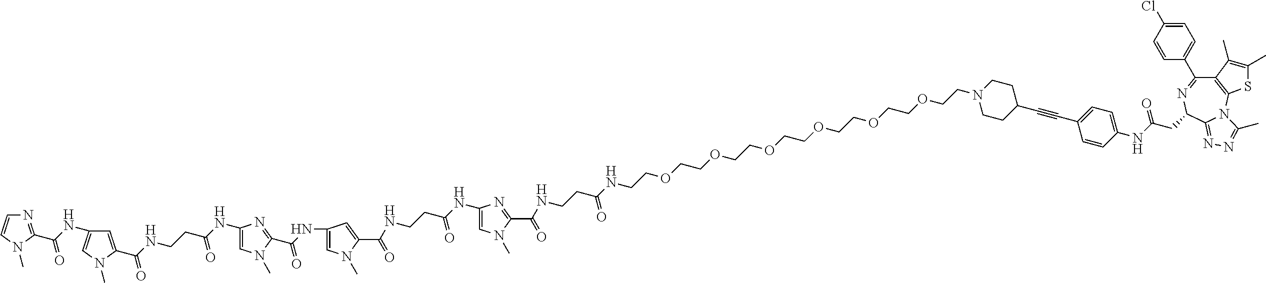 [SMILES: Cc1sc2c(c1C)C(c1ccc(Cl)cc1)=N[C@@H](CC(=O)Nc1ccc(C#CC3CCN(CCOCCOCCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)CC3)cc1)c1nnc(C)n1-2] | 11 |
| 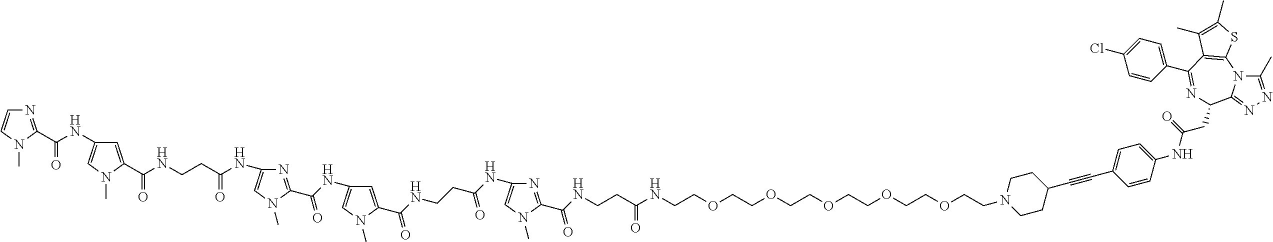 [SMILES: Cc1sc2c(c1C)C(c1ccc(Cl)cc1)=N[C@@H](CC(=O)Nc1ccc(C#CC3CCN(CCOCCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)CC3)cc1)c1nnc(C)n1-2] | 12 |
| 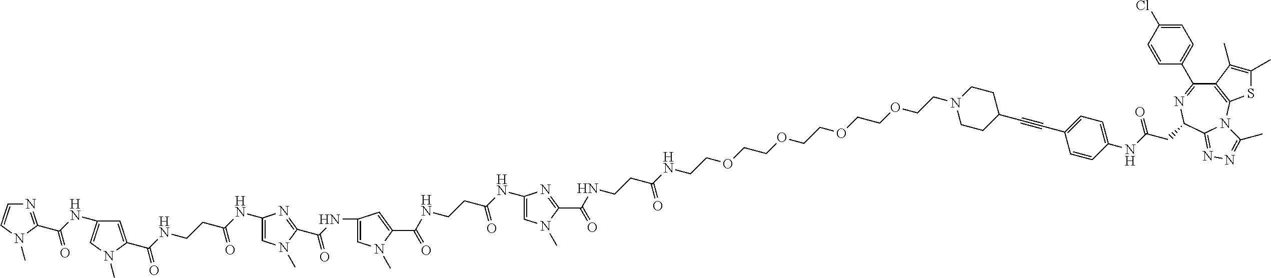 [SMILES: Cc1sc2c(c1C)C(c1ccc(Cl)cc1)=N[C@@H](CC(=O)Nc1ccc(C#CC3CCN(CCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)CC3)cc1)c1nnc(C)n1-2] | 13 |
| 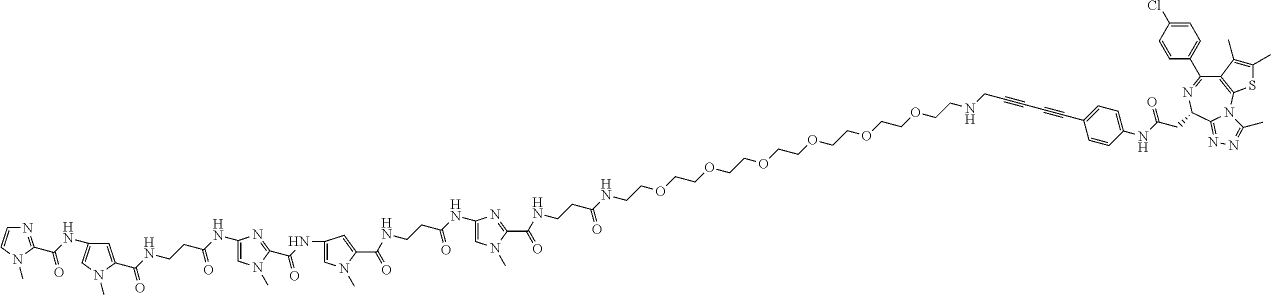 [SMILES: Cc1sc2c(c1C)C(c1ccc(Cl)cc1)=N[C@@H](CC(=O)Nc1ccc(C#CC#CCNCCOCCOCCOCCOCCOCCOCCNC(=O)CCNC(=O)c3nc(NC(=O)CCNC(=O)c4cc(NC(=O)c5nc(NC(=O)CCNC(=O)c6cc(NC(=O)c7nccn7C)cn6C)cn5C)cn4C)cn3C)cc1)c1nnc(C)n1-2] | 14 |
| 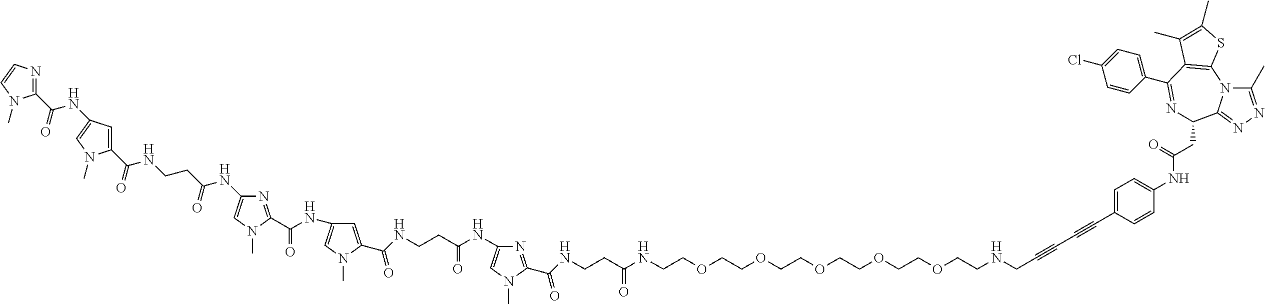 [SMILES: Cc1sc2c(c1C)C(c1ccc(Cl)cc1)=N[C@@H](CC(=O)Nc1ccc(C#CC#CCNCCOCCOCCOCCOCCOCCNC(=O)CCNC(=O)c3nc(NC(=O)CCNC(=O)c4cc(NC(=O)c5nc(NC(=O)CCNC(=O)c6cc(NC(=O)c7nccn7C)cn6C)cn5C)cn4C)cn3C)cc1)c1nnc(C)n1-2] | 15 |
| 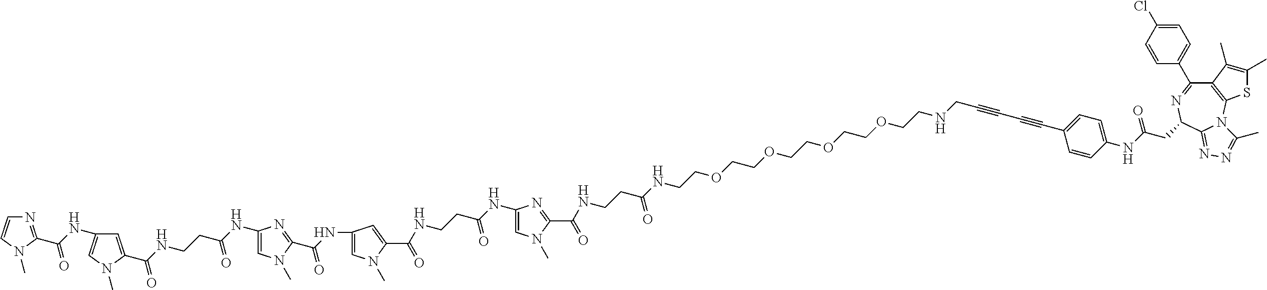 [SMILES: Cc1sc2c(c1C)C(c1ccc(Cl)cc1)=N[C@@H](CC(=O)Nc1ccc(C#CC#CCNCCOCCOCCOCCOCCNC(=O)CCNC(=O)c3nc(NC(=O)CCNC(=O)c4cc(NC(=O)c5nc(NC(=O)CCNC(=O)c6cc(NC(=O)c7nccn7C)cn6C)cn5C)cn4C)cn3C)cc1)c1nnc(C)n1-2] | 16 |
| 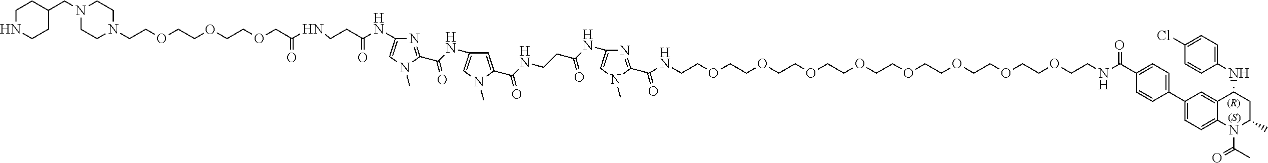 | 17 |
| 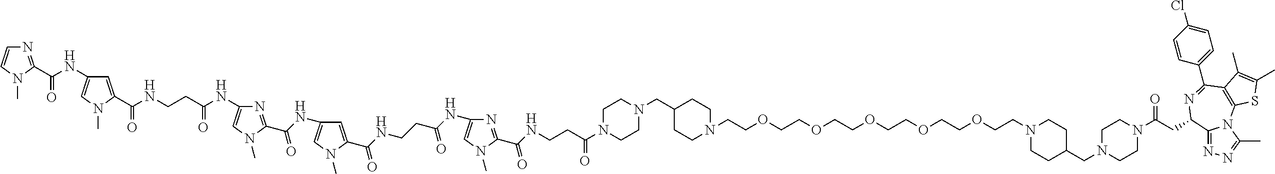 [SMILES: Cc1sc2c(c1C)C(c1ccc(Cl)cc1)=N[C@@H](CC(=O)N1CCN(CC3CCN(CCOCCOCCOCCOCCOCCN4CCC(CN5CCN(C(=O)CCNC(=O)c6nc(NC(=O)CCNC(=O)c7cc(NC(=O)c8nc(NC(=O)CCNC(=O)c9cc(NC(=O)c%10nccn%10C)cn9C)cn8C)cn7C)cn6C)CC5)CC4)CC3)CC1)c1nnc(C)n1-2] | 18 |
| 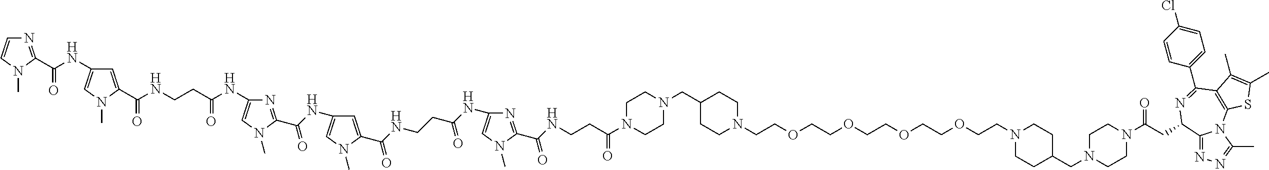 [SMILES: Cc1sc2c(c1C)C(c1ccc(Cl)cc1)=N[C@@H](CC(=O)N1CCN(CC3CCN(CCOCCOCCOCCOCCN4CCC(CN5CCN(C(=O)CCNC(=O)c6nc(NC(=O)CCNC(=O)c7cc(NC(=O)c8nc(NC(=O)CCNC(=O)c9cc(NC(=O)c%10nccn%10C)cn9C)cn8C)cn7C)cn6C)CC5)CC4)CC3)CC1)c1nnc(C)n1-2] | 19 |
| 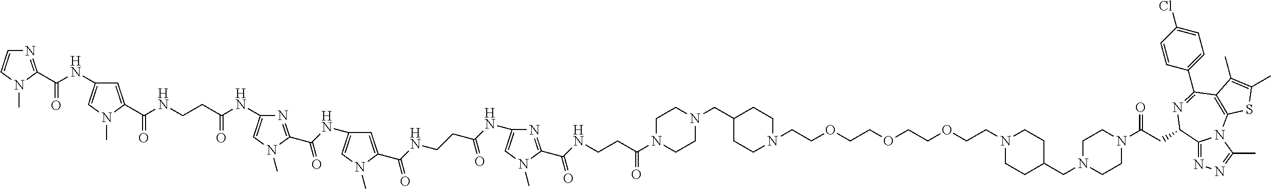 [SMILES: Cc1sc2c(c1C)C(c1ccc(Cl)cc1)=N[C@@H](CC(=O)N1CCN(CC3CCN(CCOCCOCCOCCN4CCC(CN5CCN(C(=O)CCNC(=O)c6nc(NC(=O)CCNC(=O)c7cc(NC(=O)c8nc(NC(=O)CCNC(=O)c9cc(NC(=O)c%10nccn%10C)cn9C)cn8C)cn7C)cn6C)CC5)CC4)CC3)CC1)c1nnc(C)n1-2] | 20 |
| 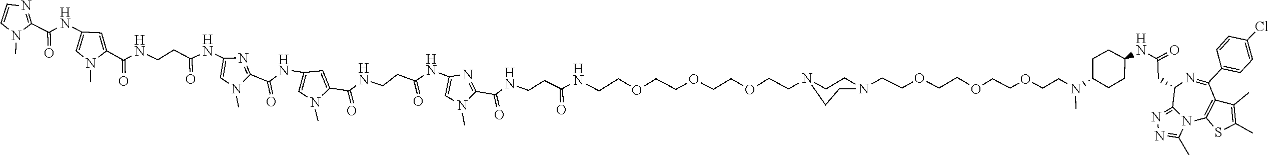 [SMILES: Cc1sc2c(c1C)C(c1ccc(Cl)cc1)=N[C@@H](CC(=O)N[C@H]1CC[C@H](N(C)CCOCCOCCOCCN3CCN(CCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)CC3)CC1)c1nnc(C)n1-2] | 21 |
| 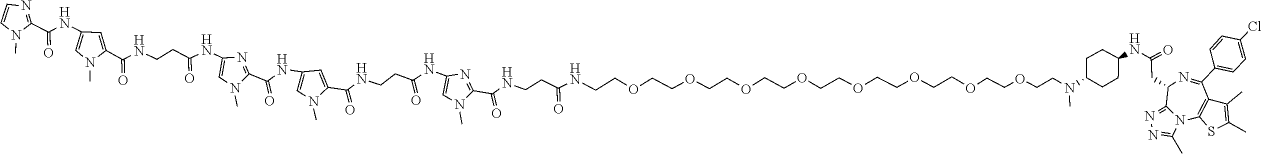 [SMILES: Cc1sc2c(c1C)C(c1ccc(Cl)cc1)=N[C@@H](CC(=O)N[C@H]1CC[C@H](N(C)CCOCCOCCOCCOCCOCCOCCOCCOCCNC(=O)CCNC(=O)c3nc(NC(=O)CCNC(=O)c4cc(NC(=O)c5nc(NC(=O)CCNC(=O)c6cc(NC(=O)c7nccn7C)cn6C)cn5C)cn4C)cn3C)CC1)c1nnc(C)n1-2] | 22 |
| 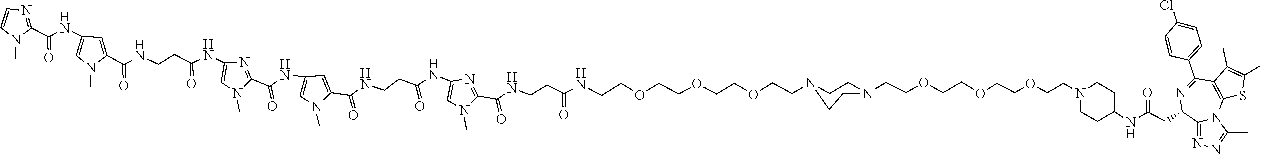 [SMILES: Cc1sc2c(c1C)C(c1ccc(Cl)cc1)=N[C@@H](CC(=O)NC1CCN(CCOCCOCCOCCN3CCN(CCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)CC3)CC1)c1nnc(C)n1-2] | 23 |
| 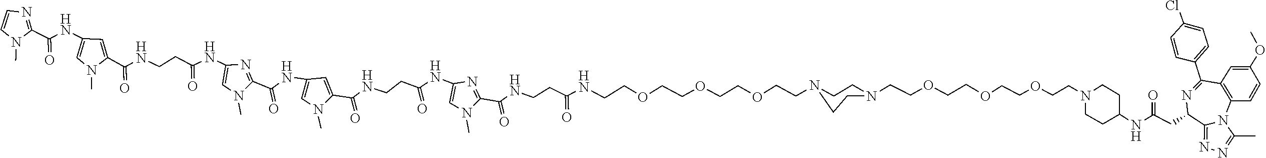 [SMILES: COc1ccc2c(c1)C(c1ccc(Cl)cc1)=N[C@@H](CC(=O)NC1CCN(CCOCCOCCOCCN3CCN(CCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)CC3)CC1)c1nnc(C)n1-2] | 24 |
| 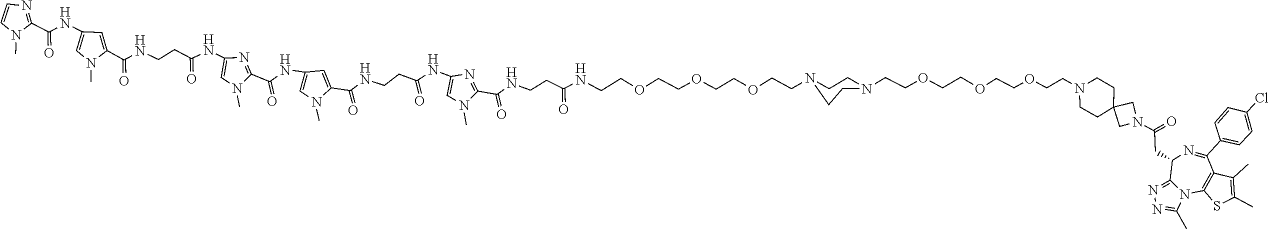 [SMILES: Cc1sc2c(c1C)C(c1ccc(Cl)cc1)=N[C@@H](CC(=O)N1CC3(CCN(CCOCCOCCOCCN4CCN(CCOCCOCCOCCNC(=O)CCNC(=O)c5nc(NC(=O)CCNC(=O)c6cc(NC(=O)c7nc(NC(=O)CCNC(=O)c8cc(NC(=O)c9nccn9C)cn8C)cn7C)cn6C)cn5C)CC4)CC3)C1)c1nnc(C)n1-2] | 25 |
| 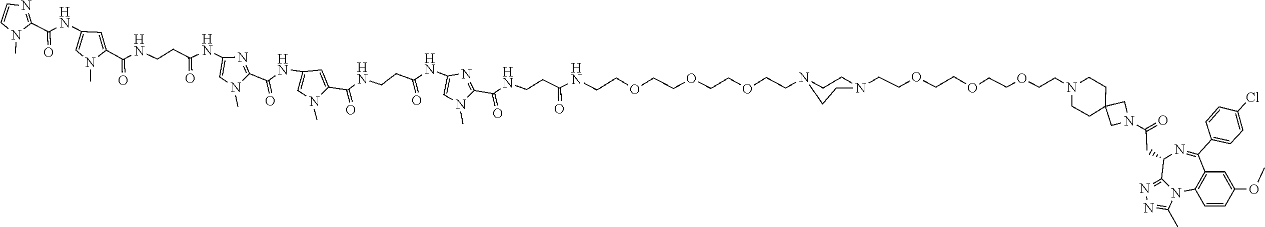 [SMILES: COc1ccc2c(c1)C(c1ccc(Cl)cc1)=N[C@@H](CC(=O)N1CC3(CCN(CCOCCOCCOCCN4CCN(CCOCCOCCOCCNC(=O)CCNC(=O)c5nc(NC(=O)CCNC(=O)c6cc(NC(=O)c7nc(NC(=O)CCNC(=O)c8cc(NC(=O)c9nccn9C)cn8C)cn7C)cn6C)cn5C)CC4)CC3)C1)c1nnc(C)n1-2] | 26 |
| 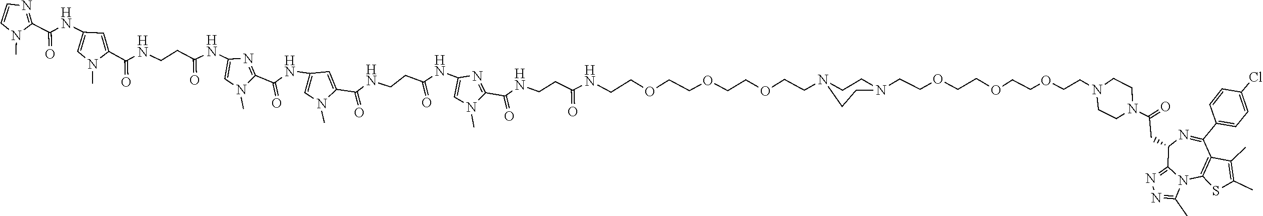 [SMILES: Cc1sc2c(c1C)C(c1ccc(Cl)cc1)=N[C@@H](CC(=O)N1CCN(CCOCCOCCOCCN3CCN(CCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)CC3)CC1)c1nnc(C)n1-2] | 27 |
| 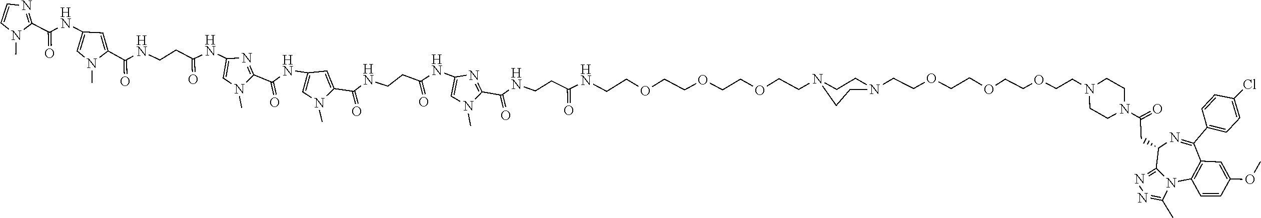 [SMILES: COc1ccc2c(c1)C(c1ccc(Cl)cc1)=N[C@@H](CC(=O)N1CCN(CCOCCOCCOCCN3CCN(CCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)CC3)CC1)c1nnc(C)n1-2] | 28 |
| 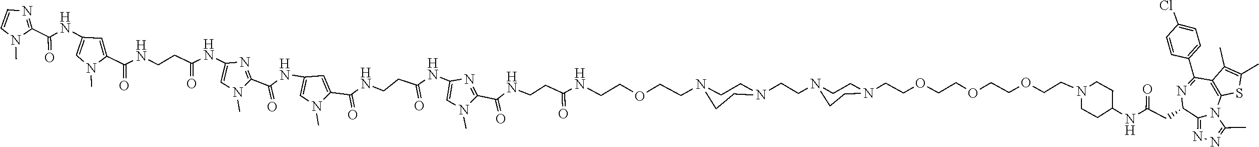 [SMILES: Cc1sc2c(c1C)C(c1ccc(Cl)cc1)=N[C@@H](CC(=O)NC1CCN(CCOCCOCCOCCN3CCN(CCN4CCN(CCOCCNC(=O)CCNC(=O)c5nc(NC(=O)CCNC(=O)c6cc(NC(=O)c7nc(NC(=O)CCNC(=O)c8cc(NC(=O)c9nccn9C)cn8C)cn7C)cn6C)cn5C)CC4)CC3)CC1)c1nnc(C)n1-2] | 29 |
| 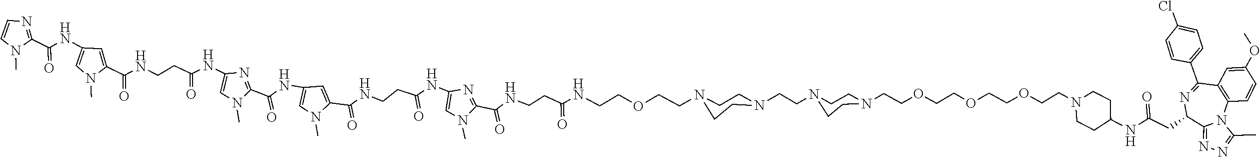 [SMILES: COc1ccc2c(c1)C(c1ccc(Cl)cc1)=N[C@@H](CC(=O)NC1CCN(CCOCCOCCOCCN3CCN(CCN4CCN(CCOCCNC(=O)CCNC(=O)c5nc(NC(=O)CCNC(=O)c6cc(NC(=O)c7nc(NC(=O)CCNC(=O)c8cc(NC(=O)c9nccn9C)cn8C)cn7C)cn6C)cn5C)CC4)CC3)CC1)c1nnc(C)n1-2] | 30 |
| 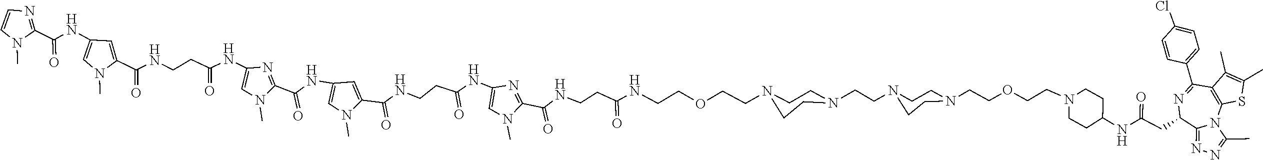 [SMILES: Cc1sc2c(c1C)C(c1ccc(Cl)cc1)=N[C@@H](CC(=O)NC1CCN(CCOCCN3CCN(CCN4CCN(CCOCCNC(=O)CCNC(=O)c5nc(NC(=O)CCNC(=O)c6cc(NC(=O)c7nc(NC(=O)CCNC(=O)c8cc(NC(=O)c9nccn9C)cn8C)cn7C)cn6C)cn5C)CC4)CC3)CC1)c1nnc(C)n1-2] | 31 |
| 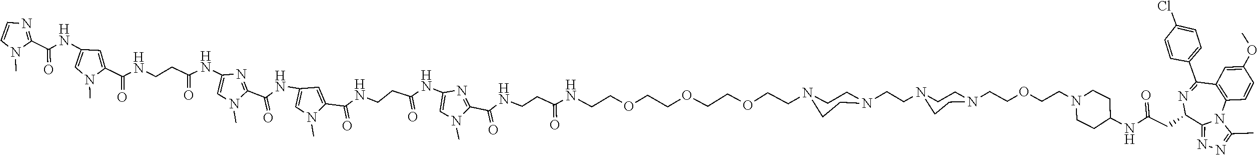 [SMILES: COc1ccc2c(c1)C(c1ccc(Cl)cc1)=N[C@@H](CC(=O)NC1CCN(CCOCCN3CCN(CCN4CCN(CCOCCOCCOCCNC(=O)CCNC(=O)c5nc(NC(=O)CCNC(=O)c6cc(NC(=O)c7nc(NC(=O)CCNC(=O)c8cc(NC(=O)c9nccn9C)cn8C)cn7C)cn6C)cn5C)CC4)CC3)CC1)c1nnc(C)n1-2] | 32 |
| 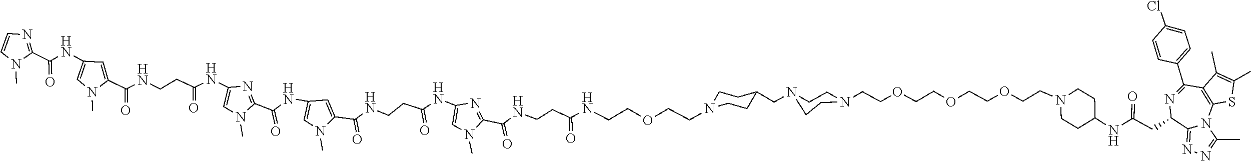 [SMILES: Cc1sc2c(c1C)C(c1ccc(Cl)cc1)=N[C@@H](CC(=O)NC1CCN(CCOCCOCCOCCN3CCN(CC4CCN(CCOCCNC(=O)CCNC(=O)c5nc(NC(=O)CCNC(=O)c6cc(NC(=O)c7nc(NC(=O)CCNC(=O)c8cc(NC(=O)c9nccn9C)cn8C)cn7C)cn6C)cn5C)CC4)CC3)CC1)c1nnc(C)n1-2] | 33 |
| 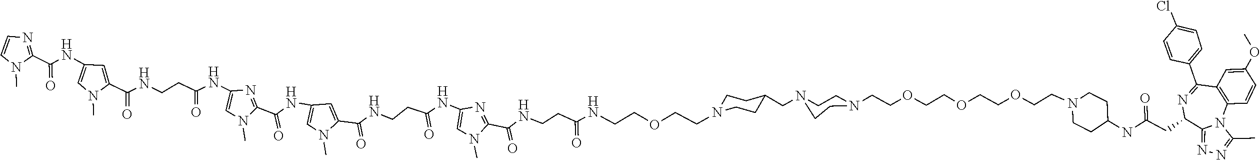 | 34 |
| 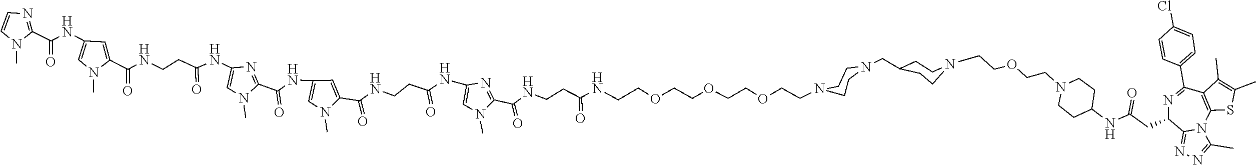 [SMILES: Cc1sc2c(c1C)C(c1ccc(Cl)cc1)=N[C@@H](CC(=O)NC1CCN(CCOCCN3CCC(CN4CCN(CCOCCOCCOCCNC(=O)CCNC(=O)c5nc(NC(=O)CCNC(=O)c6cc(NC(=O)c7nc(NC(=O)CCNC(=O)c8cc(NC(=O)c9nccn9C)cn8C)cn7C)cn6C)cn5C)CC4)CC3)CC1)c1nnc(C)n1-2] | 35 |
| 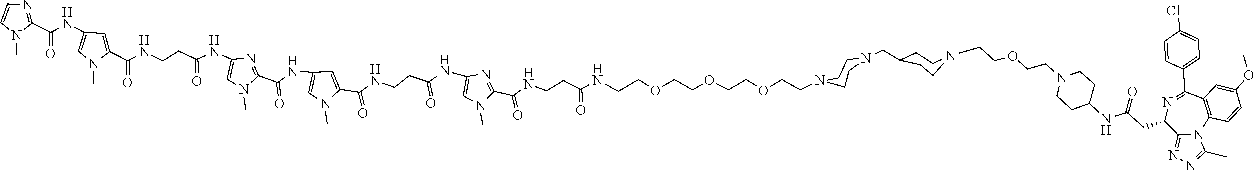 [SMILES: COc1ccc2c(c1)C(c1ccc(Cl)cc1)=N[C@@H](CC(=O)NC1CCN(CCOCCN3CCC(CN4CCN(CCOCCOCCOCCNC(=O)CCNC(=O)c5nc(NC(=O)CCNC(=O)c6cc(NC(=O)c7nc(NC(=O)CCNC(=O)c8cc(NC(=O)c9nccn9C)cn8C)cn7C)cn6C)cn5C)CC4)CC3)CC1)c1nnc(C)n1-2] | 36 |
| 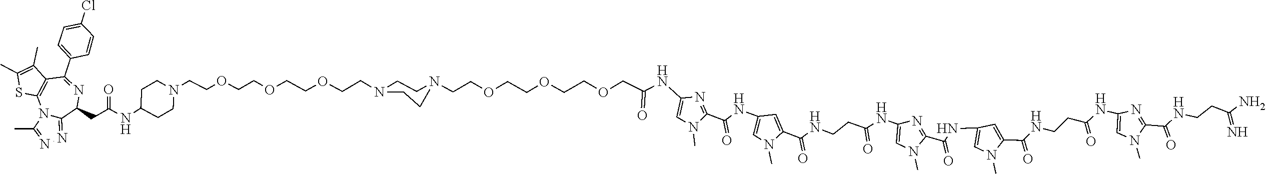 [SMILES: Cc1sc2c(c1C)C(c1ccc(Cl)cc1)=N[C@@H](CC(=O)NC1CCN(CCOCCOCCOCCN3CCN(CCOCCOCCOCC(=O)Nc4cn(C)c(C(=O)Nc5cc(C(=O)NCCC(=O)Nc6cn(C)c(C(=O)Nc7cc(C(=O)NCCC(=O)Nc8cn(C)c(C(=O)NCCC(=N)N)n8)n(C)c7)n6)n(C)c5)n4)CC3)CC1)c1nnc(C)n1-2] | 37 |
| 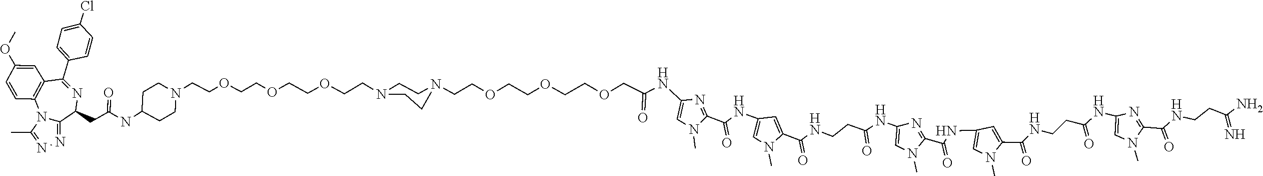 | 38 |
| 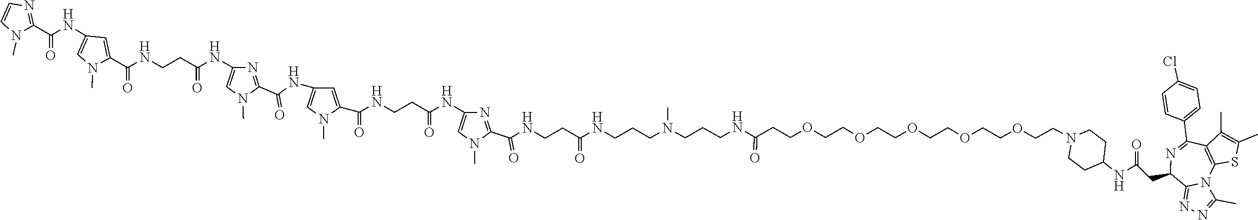 [SMILES: Cc1sc2c(c1C)C(c1ccc(Cl)cc1)=N[C@H](CC(=O)NC1CCN(CCOCCOCCOCCOCCOCCC(=O)NCCCN(C)CCCNC(=O)CCNC(=O)c3nc(NC(=O)CCNC(=O)c4cc(NC(=O)c5nc(NC(=O)CCNC(=O)c6cc(NC(=O)c7nccn7C)cn6C)cn5C)cn4C)cn3C)CC1)c1nnc(C)n1-2] | 39 |
| 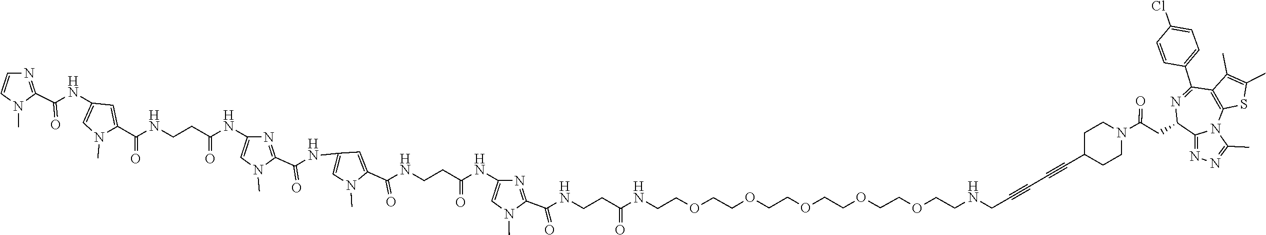 [SMILES: Cc1sc2c(c1C)C(c1ccc(Cl)cc1)=N[C@@H](CC(=O)N1CCC(C#CC#CCNCCOCCOCCOCCOCCOCCNC(=O)CCNC(=O)c3nc(NC(=O)CCNC(=O)c4cc(NC(=O)c5nc(NC(=O)CCNC(=O)c6cc(NC(=O)c7nccn7C)cn6C)cn5C)cn4C)cn3C)CC1)c1nnc(C)n1-2] | 40 |
| 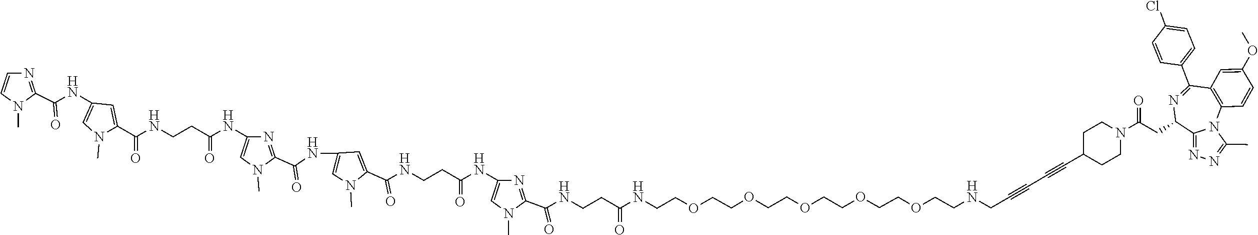 [SMILES: COc1ccc2c(c1)C(c1ccc(Cl)cc1)=N[C@@H](CC(=O)N1CCC(C#CC#CCNCCOCCOCCOCCOCCOCCNC(=O)CCNC(=O)c3nc(NC(=O)CCNC(=O)c4cc(NC(=O)c5nc(NC(=O)CCNC(=O)c6cc(NC(=O)c7nccn7C)cn6C)cn5C)cn4C)cn3C)CC1)c1nnc(C)n1-2] | 41 |
| 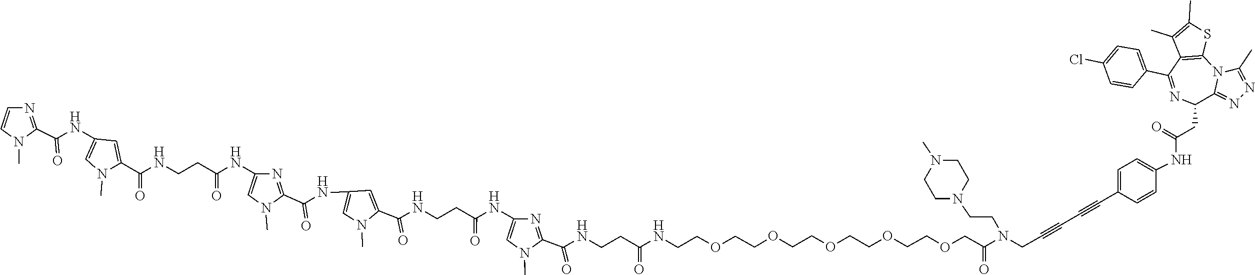 [SMILES: Cc1sc2c(c1C)C(c1ccc(Cl)cc1)=N[C@@H](CC(=O)Nc1ccc(C#CC#CCN(CCN3CCN(C)CC3)C(=O)COCCOCCOCCOCCOCCNC(=O)CCNC(=O)c3nc(NC(=O)CCNC(=O)c4cc(NC(=O)c5nc(NC(=O)CCNC(=O)c6cc(NC(=O)c7nccn7C)cn6C)cn5C)cn4C)cn3C)cc1)c1nnc(C)n1-2] | 42 |
| 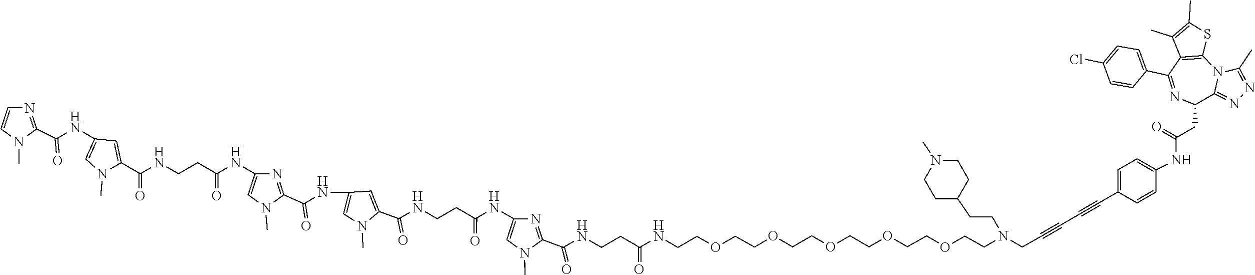 [SMILES: Cc1sc2c(c1C)C(c1ccc(Cl)cc1)=N[C@@H](CC(=O)Nc1ccc(C#CC#CCN(CCOCCOCCOCCOCCOCCNC(=O)CCNC(=O)c3nc(NC(=O)CCNC(=O)c4cc(NC(=O)c5nc(NC(=O)CCNC(=O)c6cc(NC(=O)c7nccn7C)cn6C)cn5C)cn4C)cn3C)CCC3CCN(C)CC3)cc1)c1nnc(C)n1-2] | 43 |
| 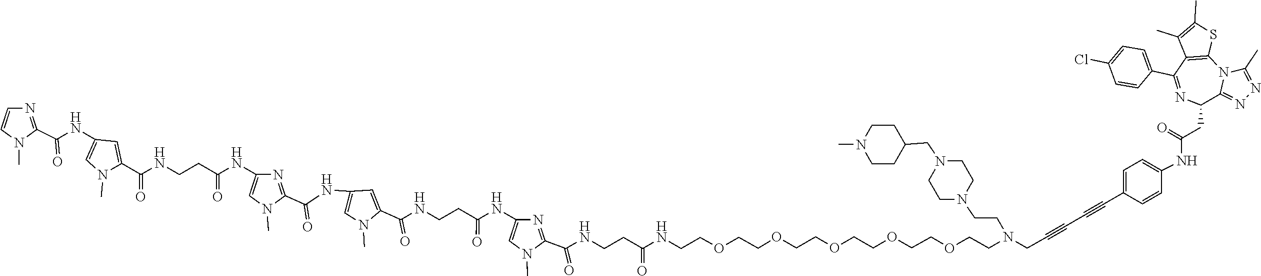 [SMILES: Cc1sc2c(c1C)C(c1ccc(Cl)cc1)=N[C@@H](CC(=O)Nc1ccc(C#CC#CCN(CCOCCOCCOCCOCCOCCNC(=O)CCNC(=O)c3nc(NC(=O)CCNC(=O)c4cc(NC(=O)c5nc(NC(=O)CCNC(=O)c6cc(NC(=O)c7nccn7C)cn6C)cn5C)cn4C)cn3C)CCN3CCN(CC4CCN(C)CC4)CC3)cc1)c1nnc(C)n1-2] | 44 |
| 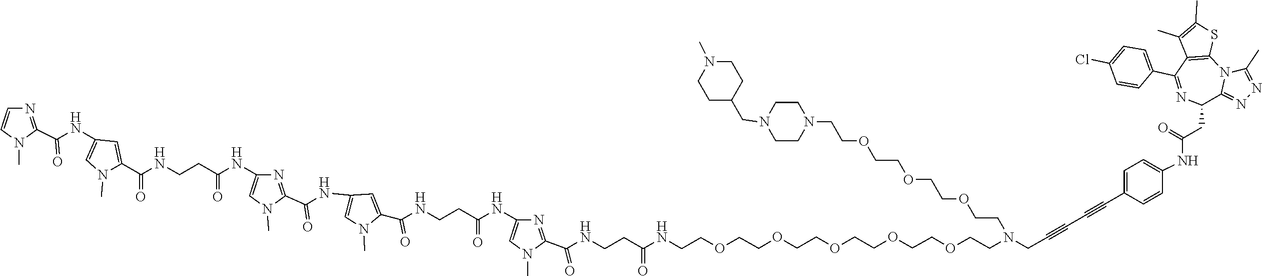 [SMILES: Cc1sc2c(c1C)C(c1ccc(Cl)cc1)=N[C@@H](CC(=O)Nc1ccc(C#CC#CCN(CCOCCOCCOCCOCCOCCNC(=O)CCNC(=O)c3nc(NC(=O)CCNC(=O)c4cc(NC(=O)c5nc(NC(=O)CCNC(=O)c6cc(NC(=O)c7nccn7C)cn6C)cn5C)cn4C)cn3C)CCOCCOCCOCCN3CCN(CC4CCN(C)CC4)CC3)cc1)c1nnc(C)n1-2] | 45 |
| 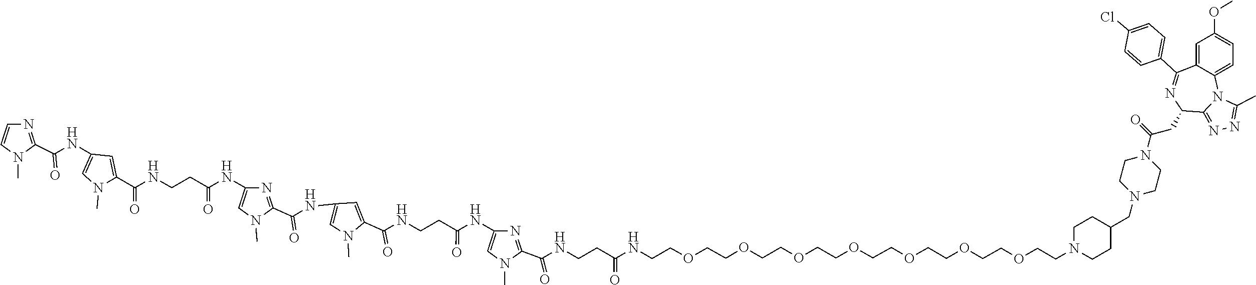 [SMILES: COc1ccc2c(c1)C(c1ccc(Cl)cc1)=N[C@@H](CC(=O)N1CCN(CC3CCN(CCOCCOCCOCCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)CC3)CC1)c1nnc(C)n1-2] | 46 |
| 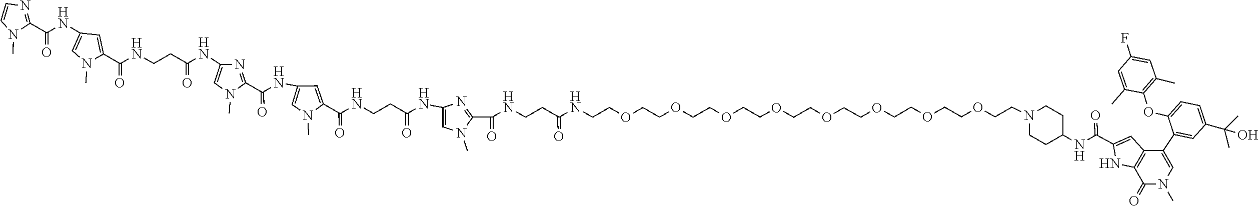 [SMILES: Cc1cc(F)cc(C)c1Oc1ccc(C(C)(C)O)cc1-c1cn(C)c(=O)c2[nH]c(C(=O)NC3CCN(CCOCCOCCOCCOCCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)CC3)cc12] | 47 |
| 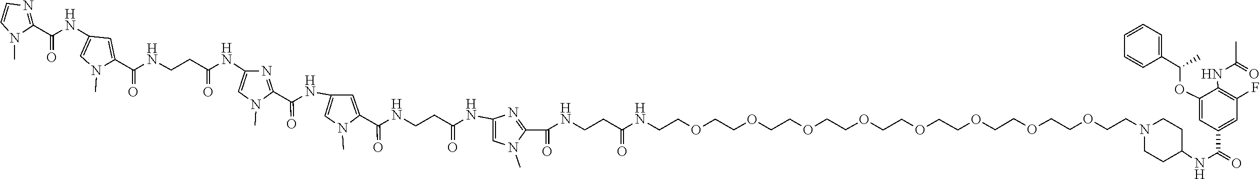 | 48 |
| 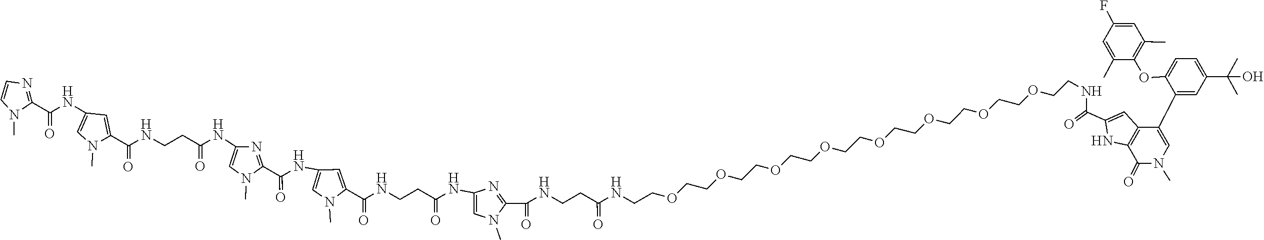 [SMILES: Cc1cc(F)cc(C)c1Oc1ccc(C(C)(C)O)cc1-c1cn(C)c(=O)c2[nH]c(C(=O)NCCOCCOCCOCCOCCOCCOCCOCCOCCNC(=O)CCNC(=O)c3nc(NC(=O)CCNC(=O)c4cc(NC(=O)c5nc(NC(=O)CCNC(=O)c6cc(NC(=O)c7nccn7C)cn6C)cn5C)cn4C)cn3C)cc12] | 50 |
| 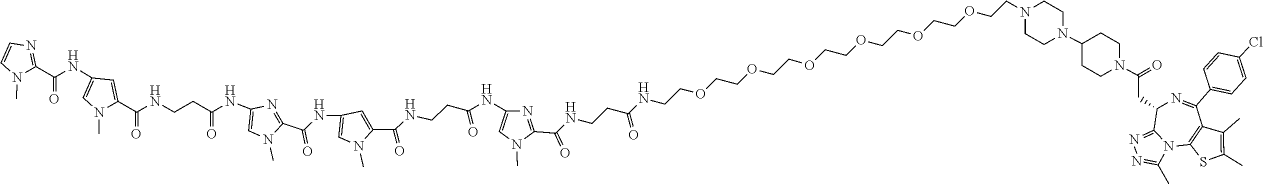 [SMILES: Cc1sc2c(c1C)C(c1ccc(Cl)cc1)=N[C@@H](CC(=O)N1CCC(N3CCN(CCOCCOCCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)CC3)CC1)c1nnc(C)n1-2] | 51 |
| 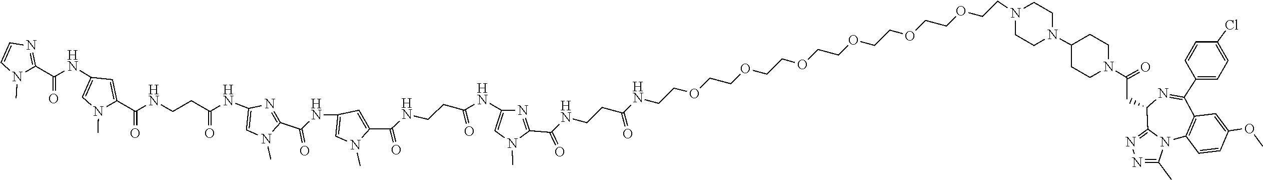 [SMILES: COc1ccc2c(c1)C(c1ccc(Cl)cc1)=N[C@@H](CC(=O)N1CCC(N3CCN(CCOCCOCCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)CC3)CC1)c1nnc(C)n1-2] | 52 |
| 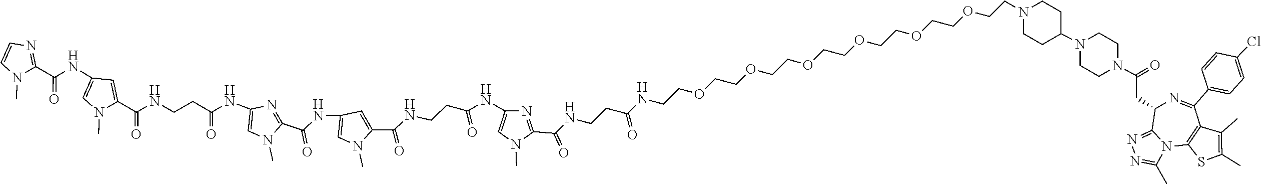 [SMILES: Cc1sc2c(c1C)C(c1ccc(Cl)cc1)=N[C@@H](CC(=O)N1CCN(C3CCN(CCOCCOCCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)CC3)CC1)c1nnc(C)n1-2] | 53 |
| 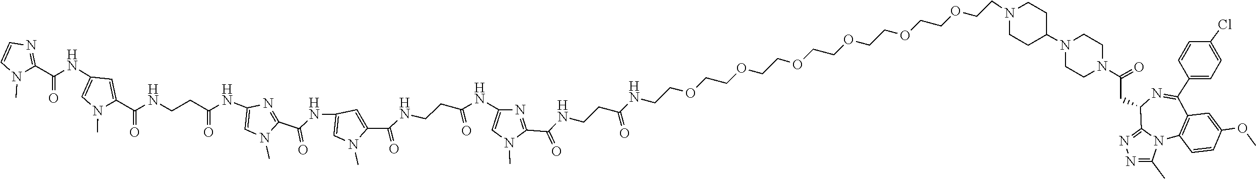 [SMILES: COc1ccc2c(c1)C(c1ccc(Cl)cc1)=N[C@@H](CC(=O)N1CCN(C3CCN(CCOCCOCCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)CC3)CC1)c1nnc(C)n1-2] | 54 |
| 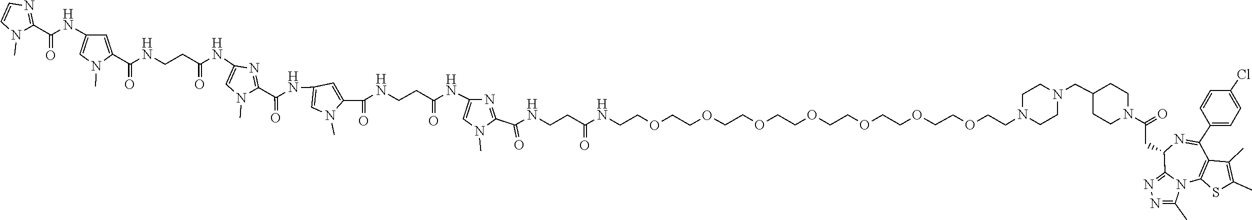 [SMILES: Cc1sc2c(c1C)C(c1ccc(Cl)cc1)=N[C@@H](CC(=O)N1CCC(CN3CCN(CCOCCOCCOCCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)CC3)CC1)c1nnc(C)n1-2] | 55 |
| 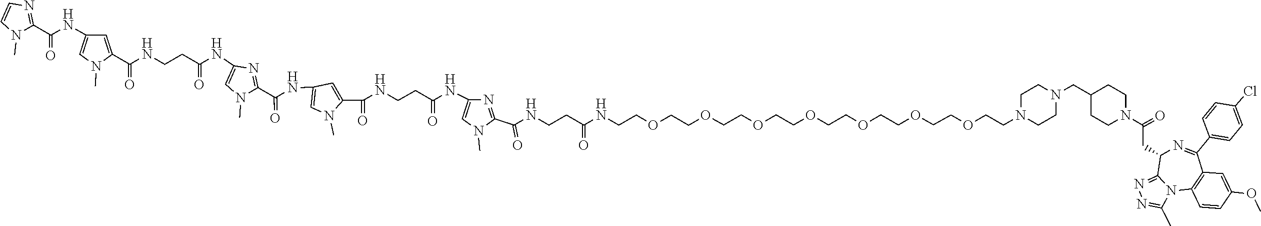 [SMILES: COc1ccc2c(c1)C(c1ccc(Cl)cc1)=N[C@@H](CC(=O)N1CCC(CN3CCN(CCOCCOCCOCCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)CC3)CC1)c1nnc(C)n1-2] | 56 |
| 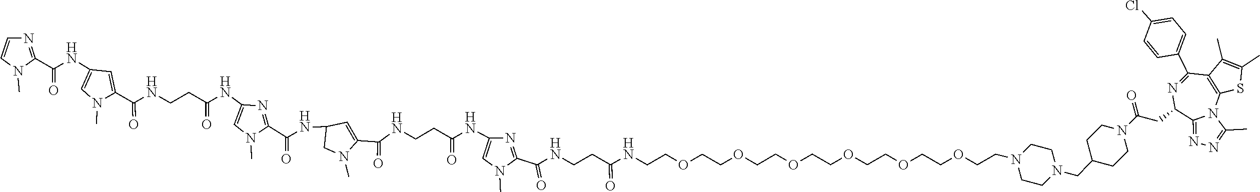 [SMILES: Cc1sc2c(c1C)C(c1ccc(Cl)cc1)=N[C@@H](CC(=O)N1CCC(CN3CCN(CCOCCOCCOCCOCCOCCOCCNC(=O)CCNC(=O)c4nc(NC(=O)CCNC(=O)C5=CC(NC(=O)c6nc(NC(=O)CCNC(=O)c7cc(NC(=O)c8nccn8C)cn7C)cn6C)CN5C)cn4C)CC3)CC1)c1nnc(C)n1-2] | 57 |
| 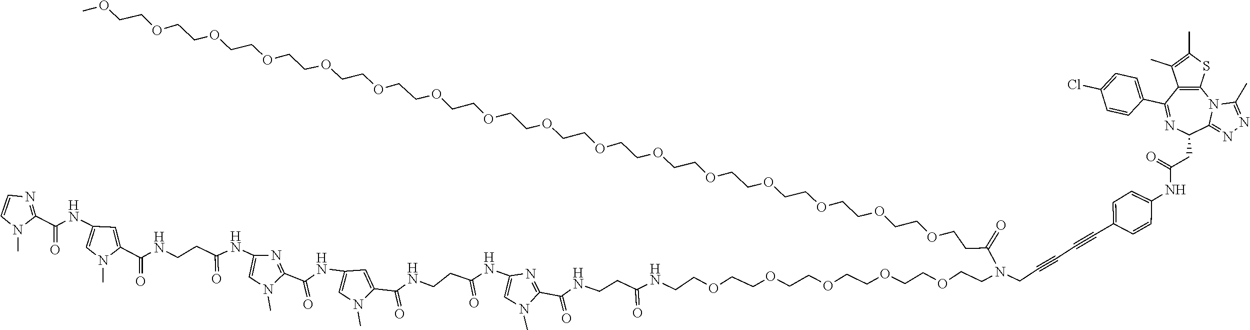 [SMILES: COCCOCCOCCOCCOCCOCCOCCOCCOCCOCCOCCOCCOCCOCCOCCOCCC(=O)N(CC#CC#Cc1ccc(NC(=O)C[C@@H]2N=C(c3ccc(Cl)cc3)c3c(sc(C)c3C)-n3c(C)nnc32)cc1)CCOCCOCCOCCOCCOCCNC(=O)CCNC(=O)c1nc(NC(=O)CCNC(=O)c2cc(NC(=O)c3nc(NC(=O)CCNC(=O)c4cc(NC(=O)c5nccn5C)cn4C)cn3C)cn2C)cn1C] | 58 |
| 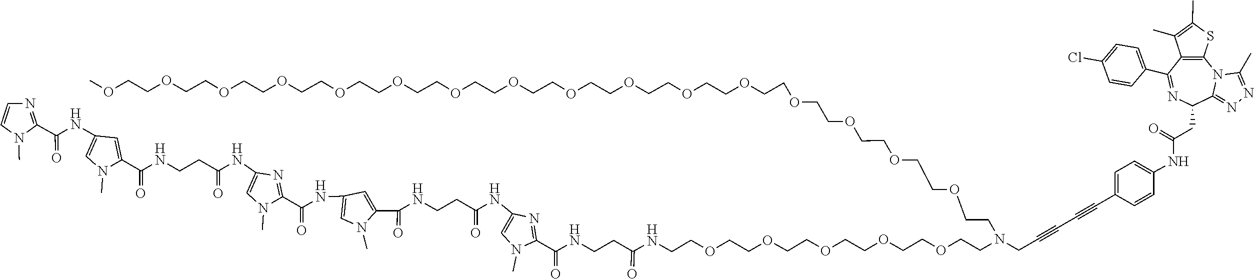 [SMILES: COCCOCCOCCOCCOCCOCCOCCOCCOCCOCCOCCOCCOCCOCCOCCOCCN(CC#CC#Cc1ccc(NC(=O)C[C@@H]2N=C(c3ccc(Cl)cc3)c3c(sc(C)c3C)-n3c(C)nnc32)cc1)CCOCCOCCOCCOCCOCCNC(=O)CCNC(=O)c1nc(NC(=O)CCNC(=O)c2cc(NC(=O)c3nc(NC(=O)CCNC(=O)c4cc(NC(=O)c5nccn5C)cn4C)cn3C)cn2C)cn1C] | 59 |
| 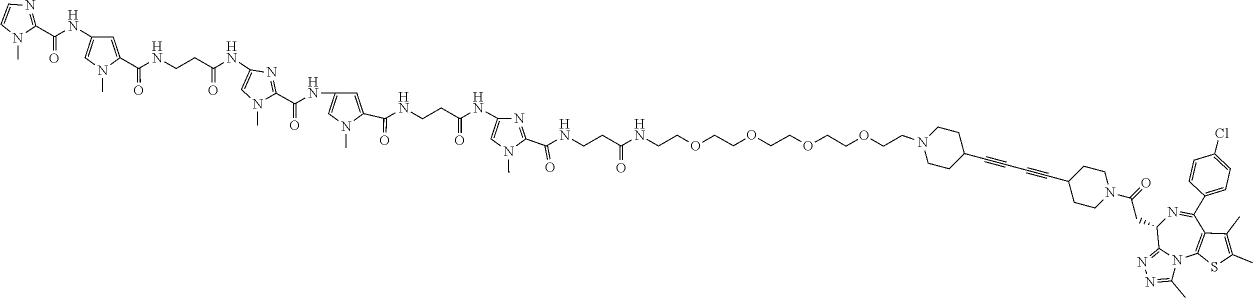 [SMILES: Cc1sc2c(c1C)C(c1ccc(Cl)cc1)=N[C@@H](CC(=O)N1CCC(C#CC#CC3CCN(CCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)CC3)CC1)c1nnc(C)n1-2] | 60 |
| 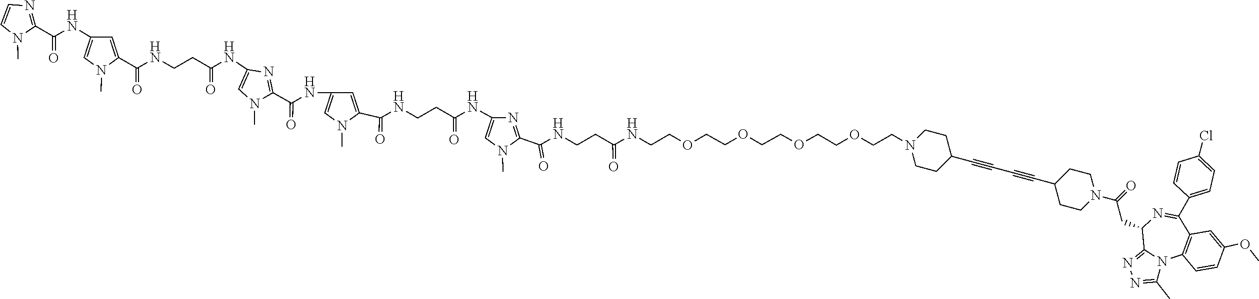 [SMILES: COc1ccc2c(c1)C(c1ccc(Cl)cc1)=N[C@@H](CC(=O)N1CCC(C#CC#CC3CCN(CCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)CC3)CC1)c1nnc(C)n1-2] | 61 |
| 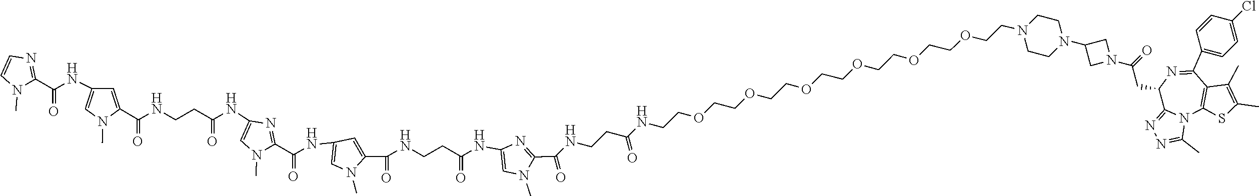 [SMILES: Cc1sc2c(c1C)C(c1ccc(Cl)cc1)=N[C@@H](CC(=O)N1CC(N3CCN(CCOCCOCCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)CC3)C1)c1nnc(C)n1-2] | 62 |
| 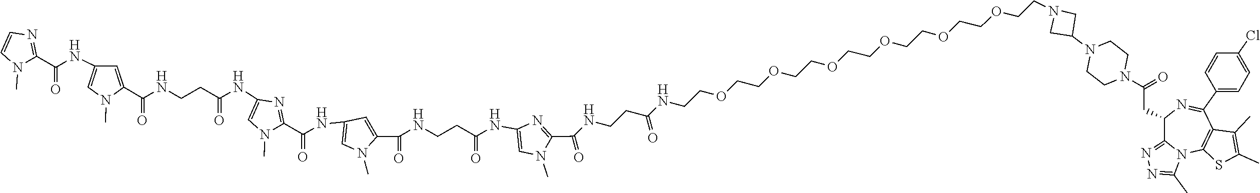 [SMILES: Cc1sc2c(c1C)C(c1ccc(Cl)cc1)=N[C@@H](CC(=O)N1CCN(C3CN(CCOCCOCCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)C3)CC1)c1nnc(C)n1-2] | 63 |
| 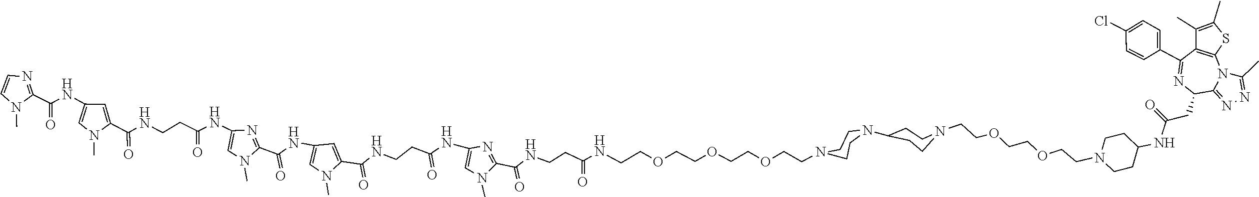 [SMILES: Cc1sc2c(c1C)C(c1ccc(Cl)cc1)=N[C@@H](CC(=O)NC1CCN(CCOCCOCCN3CCC(N4CCN(CCOCCOCCOCCNC(=O)CCNC(=O)c5nc(NC(=O)CCNC(=O)c6cc(NC(=O)c7nc(NC(=O)CCNC(=O)c8cc(NC(=O)c9nccn9C)cn8C)cn7C)cn6C)cn5C)CC4)CC3)CC1)c1nnc(C)n1-2] | 64 |
| 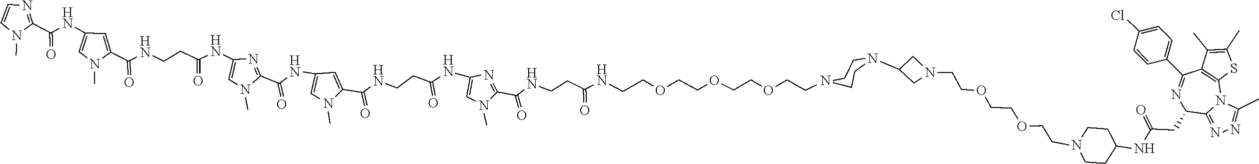 [SMILES: Cc1sc2c(c1C)C(c1ccc(Cl)cc1)=N[C@@H](CC(=O)NC1CCN(CCOCCOCCN3CC(N4CCN(CCOCCOCCOCCNC(=O)CCNC(=O)c5nc(NC(=O)CCNC(=O)c6cc(NC(=O)c7nc(NC(=O)CCNC(=O)c8cc(NC(=O)c9nccn9C)cn8C)cn7C)cn6C)cn5C)CC4)C3)CC1)c1nnc(C)n1-2] | 65 |
| 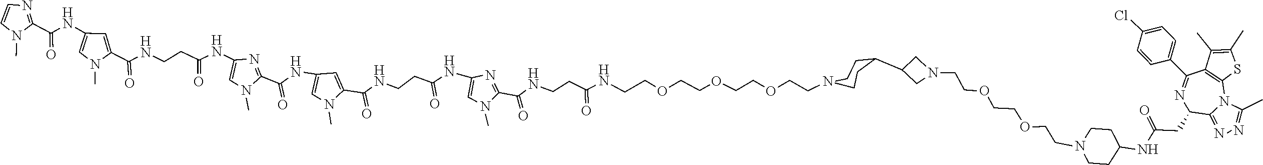 [SMILES: Cc1sc2c(c1C)C(c1ccc(Cl)cc1)=N[C@@H](CC(=O)NC1CCN(CCOCCOCCN3CC(C4CCN(CCOCCOCCOCCNC(=O)CCNC(=O)c5nc(NC(=O)CCNC(=O)c6cc(NC(=O)c7nc(NC(=O)CCNC(=O)c8cc(NC(=O)c9nccn9C)cn8C)cn7C)cn6C)cn5C)CC4)C3)CC1)c1nnc(C)n1-2] | 66 |
| 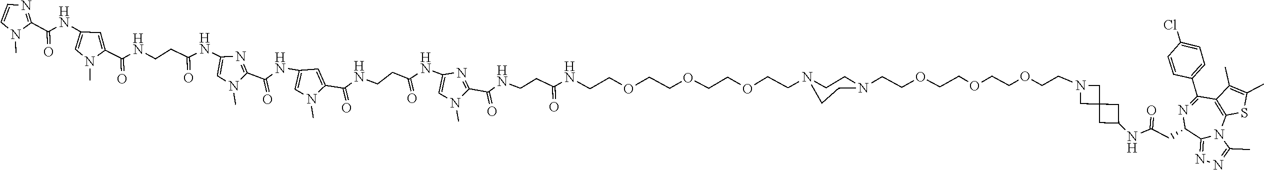 [SMILES: Cc1sc2c(c1C)C(c1ccc(Cl)cc1)=N[C@@H](CC(=O)NC1CC3(C1)CN(CCOCCOCCOCCN1CCN(CCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)CC1)C3)c1nnc(C)n1-2] | 67 |
| 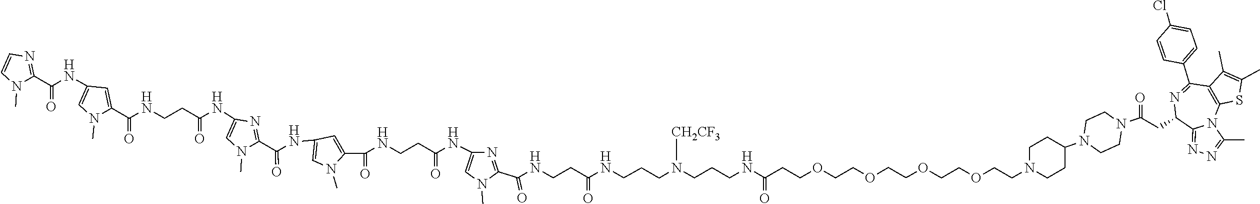 | 68 |
| 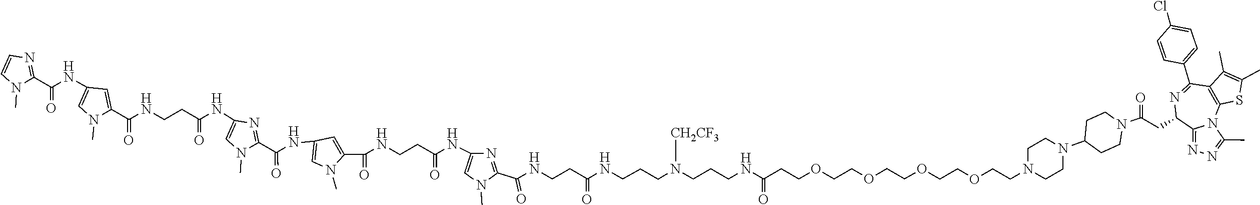 | 69 |
| 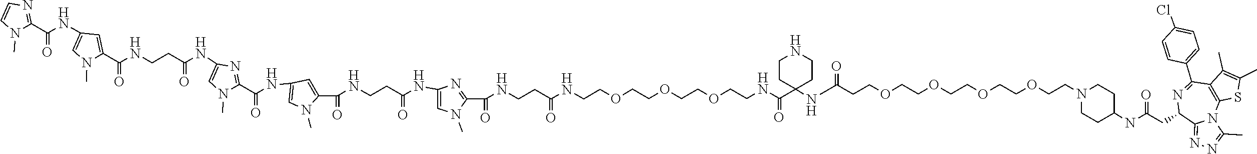 | 70 |
| 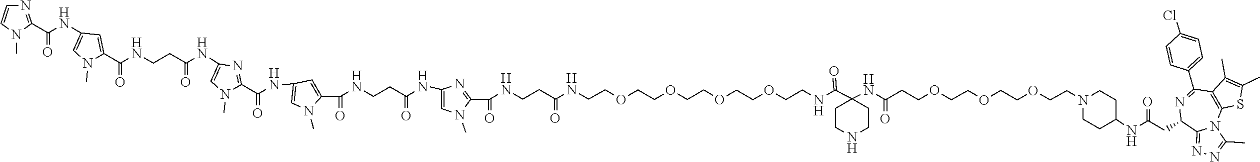 [SMILES: Cc1sc2c(c1C)C(c1ccc(Cl)cc1)=N[C@@H](CC(=O)NC1CCN(CCOCCOCCOCCC(=O)NC3(C(=O)NCCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)CCNCC3)CC1)c1nnc(C)n1-2] | 71 |
| 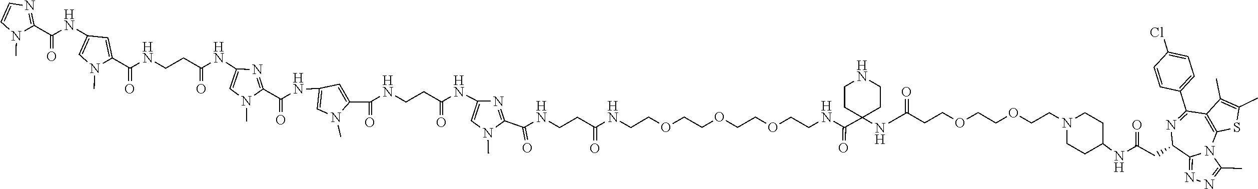 [SMILES: Cc1sc2c(c1C)C(c1ccc(Cl)cc1)=N[C@@H](CC(=O)NC1CCN(CCOCCOCCC(=O)NC3(C(=O)NCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)CCNCC3)CC1)c1nnc(C)n1-2] | 72 |
| 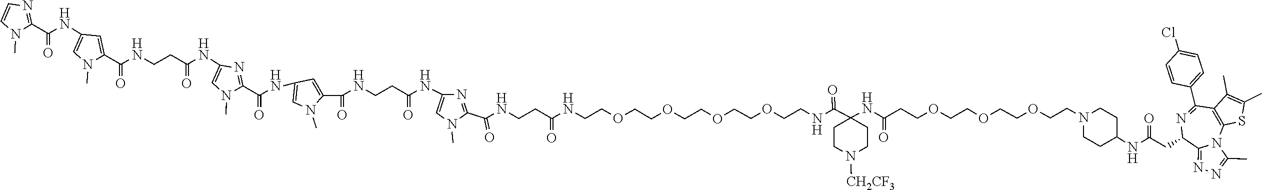 | 73 |
| 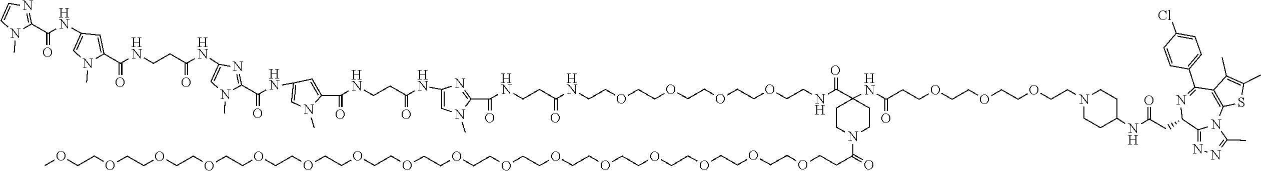 [SMILES: COCCOCCOCCOCCOCCOCCOCCOCCOCCOCCOCCOCCOCCOCCOCCOCCC(=O)N1CCC(NC(=O)CCOCCOCCOCCN2CCC(NC(=O)C[C@@H]3N=C(c4ccc(Cl)cc4)c4c(sc(C)c4C)-n4c(C)nnc43)CC2)(C(=O)NCCOCCOCCOCCOCCNC(=O)CCNC(=O)c2nc(NC(=O)CCNC(=O)c3cc(NC(=O)c4nc(NC(=O)CCNC(=O)c5cc(NC(=O)c6nccn6C)cn5C)cn4C)cn3C)cn2C)CC1] | 74 |
| 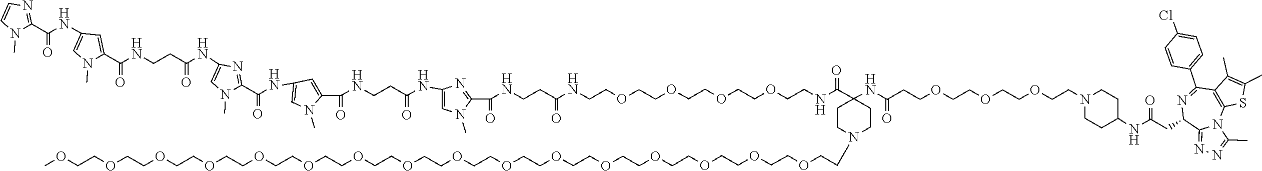 [SMILES: COCCOCCOCCOCCOCCOCCOCCOCCOCCOCCOCCOCCOCCOCCOCCOCCN1CCC(NC(=O)CCOCCOCCOCCN2CCC(NC(=O)C[C@@H]3N=C(c4ccc(Cl)cc4)c4c(sc(C)c4C)-n4c(C)nnc43)CC2)(C(=O)NCCOCCOCCOCCOCCNC(=O)CCNC(=O)c2nc(NC(=O)CCNC(=O)c3cc(NC(=O)c4nc(NC(=O)CCNC(=O)c5cc(NC(=O)c6nccn6C)cn5C)cn4C)cn3C)cn2C)CC1] | 75 |
| 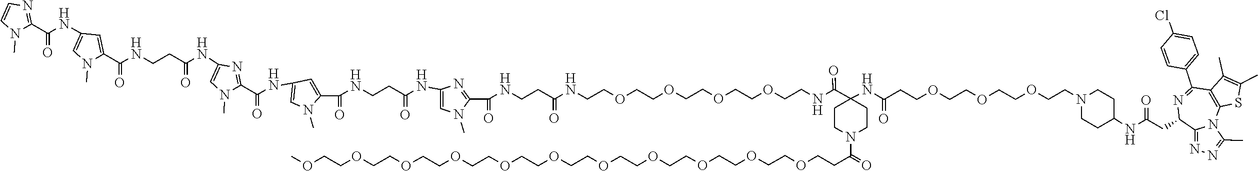 [SMILES: COCCOCCOCCOCCOCCOCCOCCOCCOCCOCCOCCC(=O)N1CCC(NC(=O)CCOCCOCCOCCN2CCC(NC(=O)C[C@@H]3N=C(c4ccc(Cl)cc4)c4c(sc(C)c4C)-n4c(C)nnc43)CC2)(C(=O)NCCOCCOCCOCCOCCNC(=O)CCNC(=O)c2nc(NC(=O)CCNC(=O)c3cc(NC(=O)c4nc(NC(=O)CCNC(=O)c5cc(NC(=O)c6nccn6C)cn5C)cn4C)cn3C)cn2C)CC1] | 76 |
| 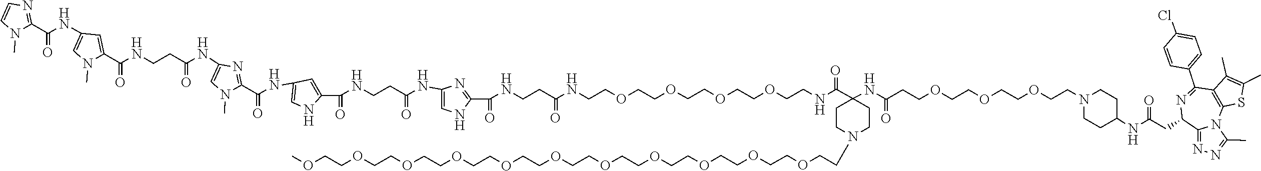 [SMILES: COCCOCCOCCOCCOCCOCCOCCOCCOCCOCCOCCN1CCC(NC(=O)CCOCCOCCOCCN2CCC(NC(=O)C[C@@H]3N=C(c4ccc(Cl)cc4)c4c(sc(C)c4C)-n4c(C)nnc43)CC2)(C(=O)NCCOCCOCCOCCOCCNC(=O)CCNC(=O)c2nc(NC(=O)CCNC(=O)c3cc(NC(=O)c4nc(NC(=O)CCNC(=O)c5cc(NC(=O)c6nccn6C)cn5C)cn4C)c[nH]3)c[nH]2)CC1] | 77 |
| 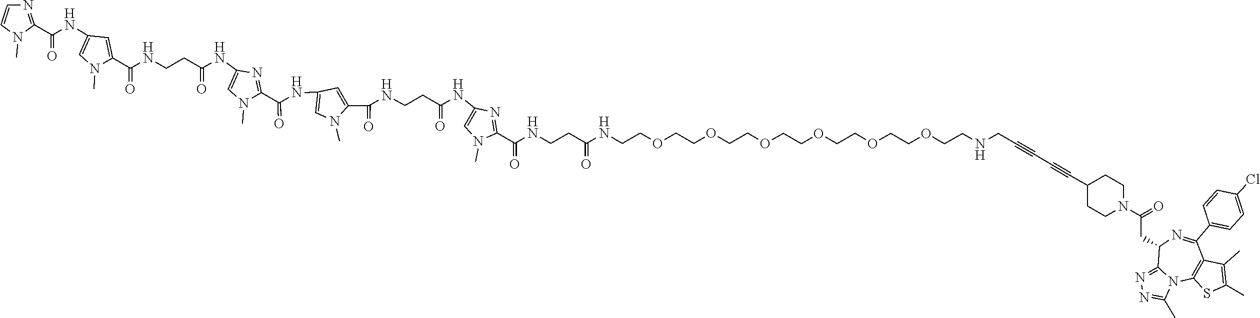 [SMILES: Cc1sc2c(c1C)C(c1ccc(Cl)cc1)=N[C@@H](CC(=O)N1CCC(C#CC#CCNCCOCCOCCOCCOCCOCCOCCNC(=O)CCNC(=O)c3nc(NC(=O)CCNC(=O)c4cc(NC(=O)c5nc(NC(=O)CCNC(=O)c6cc(NC(=O)c7nccn7C)cn6C)cn5C)cn4C)cn3C)CC1)c1nnc(C)n1-2] | 78 |
| 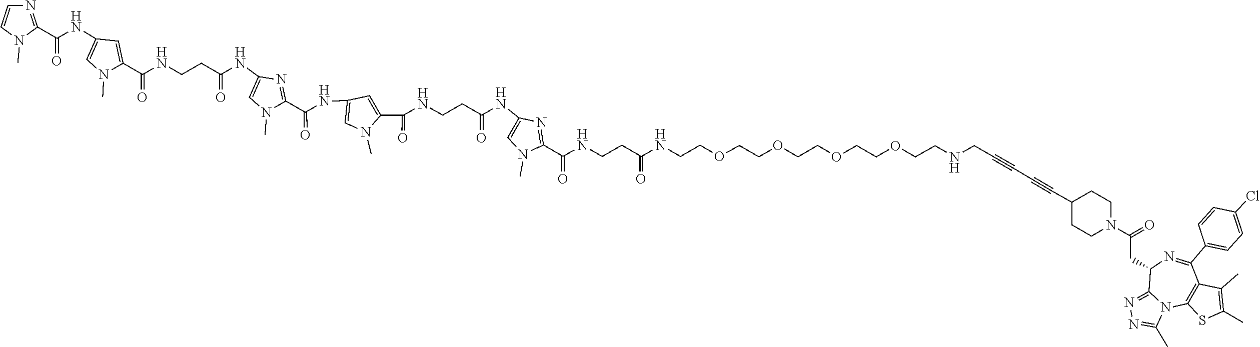 [SMILES: Cc1sc2c(c1C)C(c1ccc(Cl)cc1)=N[C@@H](CC(=O)N1CCC(C#CC#CCNCCOCCOCCOCCOCCNC(=O)CCNC(=O)c3nc(NC(=O)CCNC(=O)c4cc(NC(=O)c5nc(NC(=O)CCNC(=O)c6cc(NC(=O)c7nccn7C)cn6C)cn5C)cn4C)cn3C)CC1)c1nnc(C)n1-2] | 79 |
| 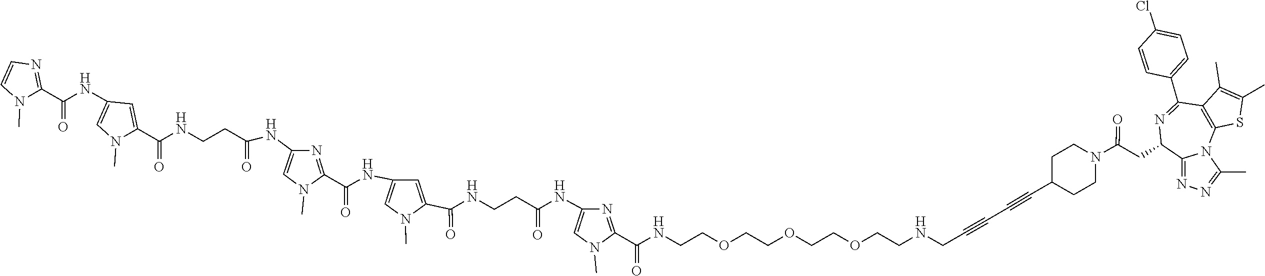 [SMILES: Cc1sc2c(c1C)C(c1ccc(Cl)cc1)=N[C@@H](CC(=O)N1CCC(C#CC#CCNCCOCCOCCOCCNC(=O)c3nc(NC(=O)CCNC(=O)c4cc(NC(=O)c5nc(NC(=O)CCNC(=O)c6cc(NC(=O)c7nccn7C)cn6C)cn5C)cn4C)cn3C)CC1)c1nnc(C)n1-2] | 80 |
| 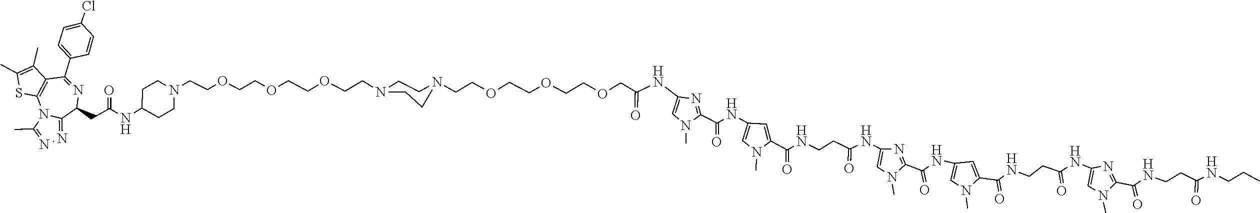 [SMILES: CCCNC(=O)CCNC(=O)c1nc(NC(=O)CCNC(=O)c2cc(NC(=O)c3nc(NC(=O)CCNC(=O)c4cc(NC(=O)c5nc(NC(=O)COCCOCCOCCN6CCN(CCOCCOCCOCCN7CCC(NC(=O)C[C@@H]8N=C(c9ccc(Cl)cc9)c9c(sc(C)c9C)-n9c(C)nnc98)CC7)CC6)cn5C)cn4C)cn3C)cn2C)cn1C] | 81 |
| 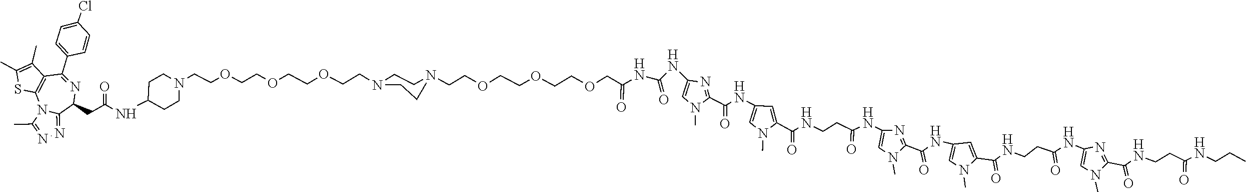 [SMILES: CCCNC(=O)CCNC(=O)c1nc(NC(=O)CCNC(=O)c2cc(NC(=O)c3nc(NC(=O)CCNC(=O)c4cc(NC(=O)c5nc(NC(=O)NC(=O)COCCOCCOCCN6CCN(CCOCCOCCOCCN7CCC(NC(=O)C[C@@H]8N=C(c9ccc(Cl)cc9)c9c(sc(C)c9C)-n9c(C)nnc98)CC7)CC6)cn5C)cn4C)cn3C)cn2C)cn1C] | 82 |
| 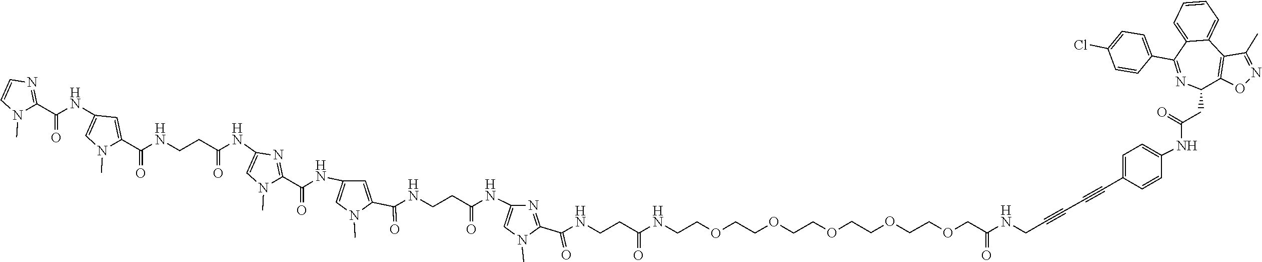 [SMILES: Cc1noc2c1-c1ccccc1C(c1ccc(Cl)cc1)=N[C@H]2CC(=O)Nc1ccc(C#CC#CCNC(=O)COCCOCCOCCOCCOCCNC(=O)CCNC(=O)c2nc(NC(=O)CCNC(=O)c3cc(NC(=O)c4nc(NC(=O)CCNC(=O)c5cc(NC(=O)c6nccn6C)cn5C)cn4C)cn3C)cn2C)cc1] | 84 |
| 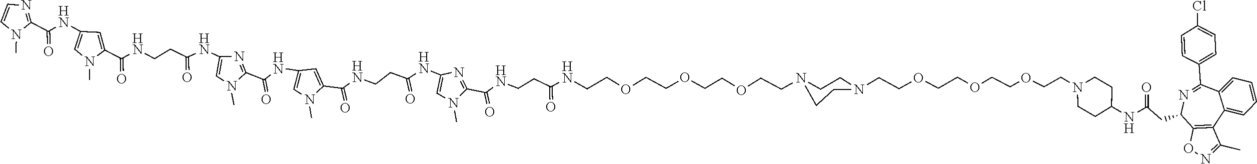 [SMILES: Cc1noc2c1-c1ccccc1C(c1ccc(Cl)cc1)=N[C@H]2CC(=O)NC1CCN(CCOCCOCCOCCN2CCN(CCOCCOCCOCCNC(=O)CCNC(=O)c3nc(NC(=O)CCNC(=O)c4cc(NC(=O)c5nc(NC(=O)CCNC(=O)c6cc(NC(=O)c7nccn7C)cn6C)cn5C)cn4C)cn3C)CC2)CC1] | 85 |
| 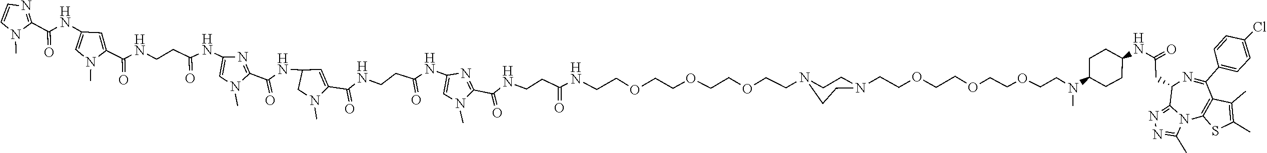 [SMILES: Cc1sc2c(c1C)C(c1ccc(Cl)cc1)=N[C@@H](CC(=O)N[C@H]1CC[C@@H](N(C)CCOCCOCCOCCN3CCN(CCOCCOCCOCCNC(=O)CCNC(=O)c4nc(NC(=O)CCNC(=O)C5=CC(NC(=O)c6nc(NC(=O)CCNC(=O)c7cc(NC(=O)c8nccn8C)cn7C)cn6C)CN5C)cn4C)CC3)CC1)c1nnc(C)n1-2] | 86 |
| 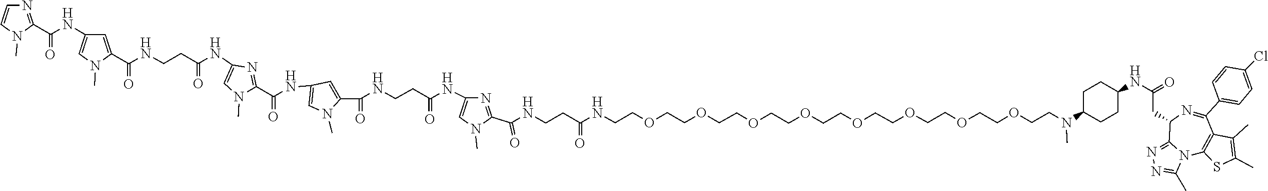 [SMILES: Cc1sc2c(c1C)C(c1ccc(Cl)cc1)=N[C@@H](CC(=O)N[C@H]1CC[C@@H](N(C)CCOCCOCCOCCOCCOCCOCCOCCOCCNC(=O)CCNC(=O)c3nc(NC(=O)CCNC(=O)c4cc(NC(=O)c5nc(NC(=O)CCNC(=O)c6cc(NC(=O)c7nccn7C)cn6C)cn5C)cn4C)cn3C)CC1)c1nnc(C)n1-2] | 87 |
| 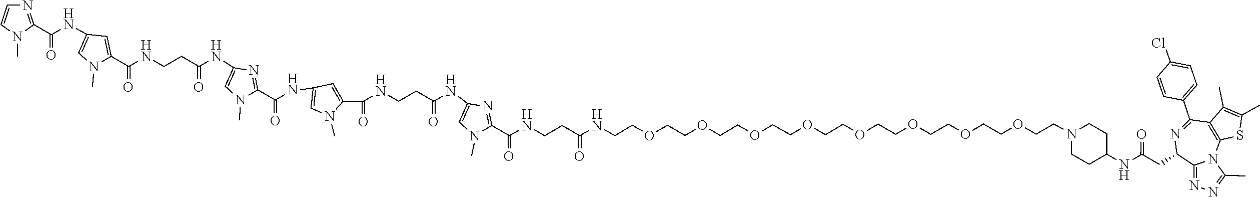 [SMILES: Cc1sc2c(c1C)C(c1ccc(Cl)cc1)=N[C@@H](CC(=O)NC1CCN(CCOCCOCCOCCOCCOCCOCCOCCOCCNC(=O)CCNC(=O)c3nc(NC(=O)CCNC(=O)c4cc(NC(=O)c5nc(NC(=O)CCNC(=O)c6cc(NC(=O)c7nccn7C)cn6C)cn5C)cn4C)cn3C)CC1)c1nnc(C)n1-2] | 88 |
| 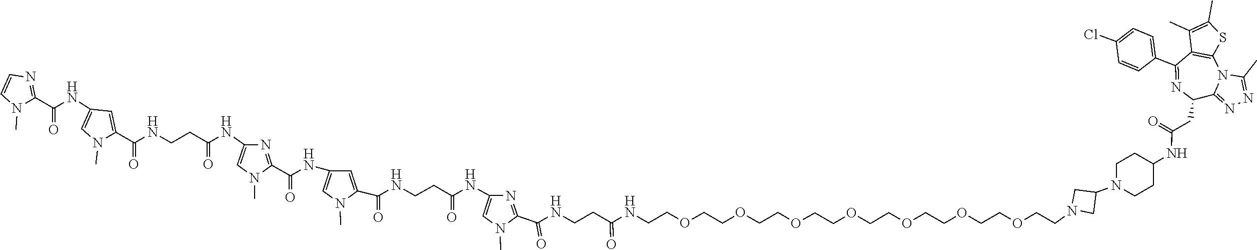 [SMILES: Cc1sc2c(c1C)C(c1ccc(Cl)cc1)=N[C@@H](CC(=O)NC1CCN(C3CN(CCOCCOCCOCCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)C3)CC1)c1nnc(C)n1-2] | 89 |
| 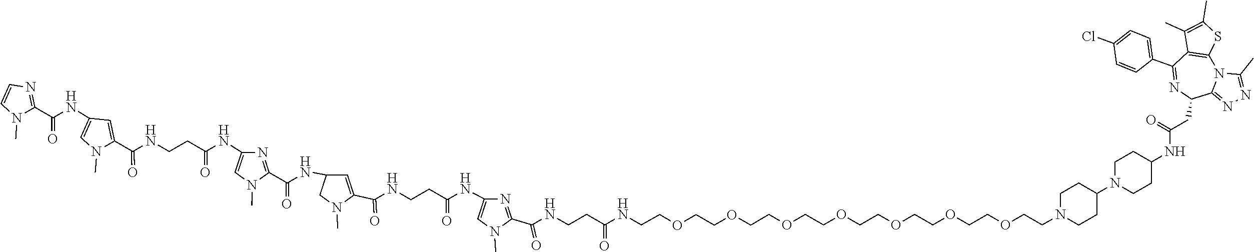 [SMILES: Cc1sc2c(c1C)C(c1ccc(Cl)cc1)=N[C@@H](CC(=O)NC1CCN(C3CCN(CCOCCOCCOCCOCCOCCOCCOCCNC(=O)CCNC(=O)c4nc(NC(=O)CCNC(=O)C5=CC(NC(=O)c6nc(NC(=O)CCNC(=O)c7cc(NC(=O)c8nccn8C)cn7C)cn6C)CN5C)cn4C)CC3)CC1)c1nnc(C)n1-2] | 90 |
| 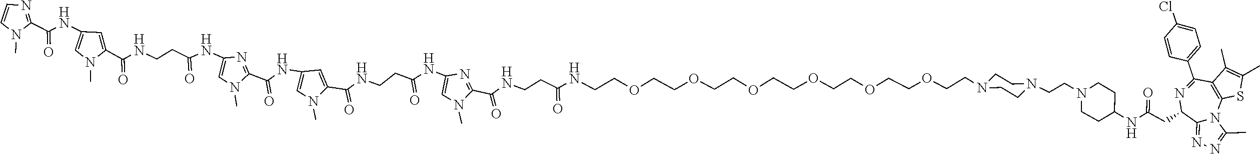 [SMILES: Cc1sc2c(c1C)C(c1ccc(Cl)cc1)=N[C@@H](CC(=O)NC1CCN(CCN3CCN(CCOCCOCCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)CC3)CC1)c1nnc(C)n1-2] | 91 |
| 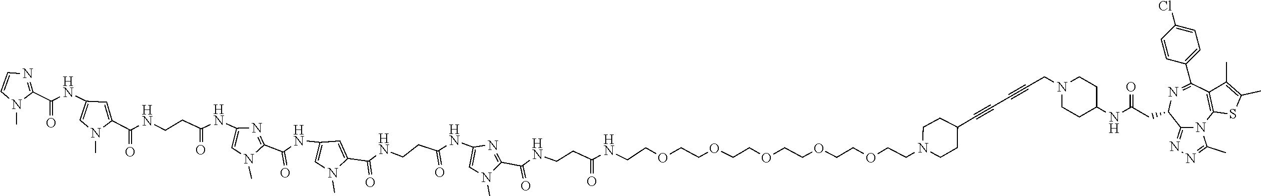 [SMILES: Cc1sc2c(c1C)C(c1ccc(Cl)cc1)=N[C@@H](CC(=O)NC1CCN(CC#CC#CC3CCN(CCOCCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)CC3)CC1)c1nnc(C)n1-2] | 92 |
| 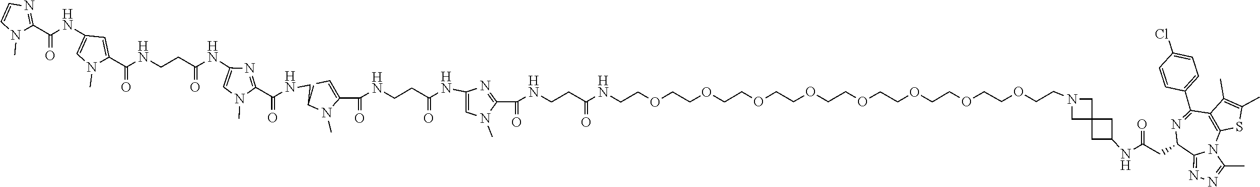 | 93 |
| 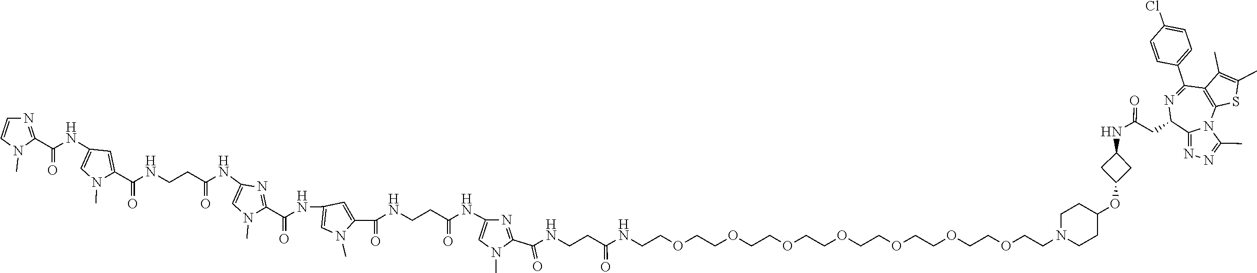 [SMILES: Cc1sc2c(c1C)C(c1ccc(Cl)cc1)=N[C@@H](CC(=O)N[C@H]1C[C@H](OC3CCN(CCOCCOCCOCCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)CC3)C1)c1nnc(C)n1-2] | 94 |
| 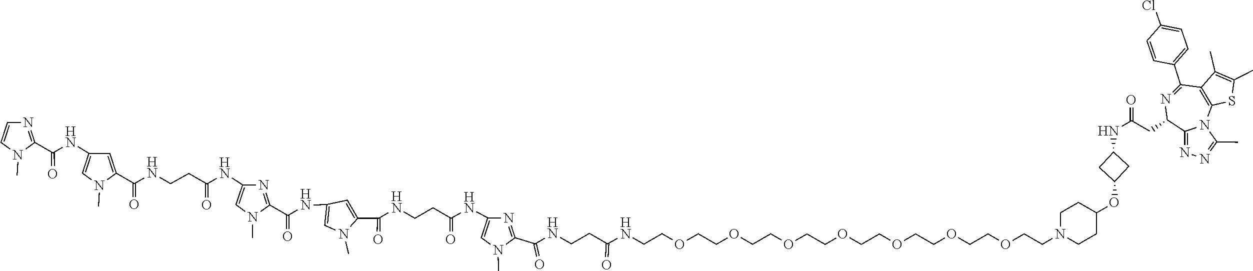 [SMILES: Cc1sc2c(c1C)C(c1ccc(Cl)cc1)=N[C@@H](CC(=O)N[C@H]1C[C@@H](OC3CCN(CCOCCOCCOCCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)CC3)C1)c1nnc(C)n1-2] | 95 |
| 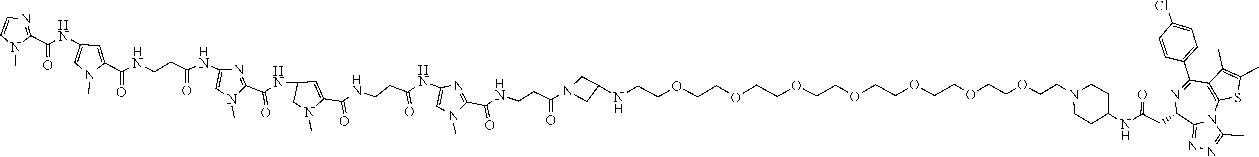 [SMILES: Cc1sc2c(c1C)C(c1ccc(Cl)cc1)=N[C@@H](CC(=O)NC1CCN(CCOCCOCCOCCOCCOCCOCCOCCNC3CN(C(=O)CCNC(=O)c4nc(NC(=O)CCNC(=O)C5=CC(NC(=O)c6nc(NC(=O)CCNC(=O)c7cc(NC(=O)c8nccn8C)cn7C)cn6C)CN5C)cn4C)C3)CC1)c1nnc(C)n1-2] | 96 |
| 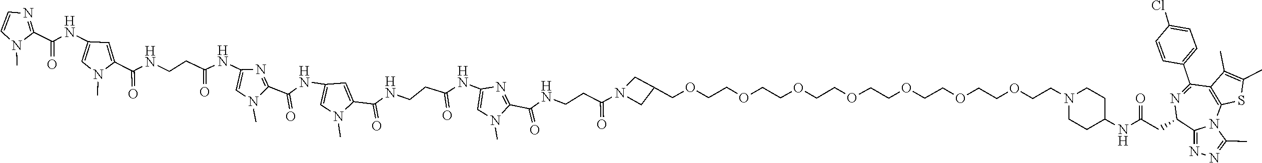 [SMILES: Cc1sc2c(c1C)C(c1ccc(Cl)cc1)=N[C@@H](CC(=O)NC1CCN(CCOCCOCCOCCOCCOCCOCCOCC3CN(C(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)C3)CC1)c1nnc(C)n1-2] | 97 |
| 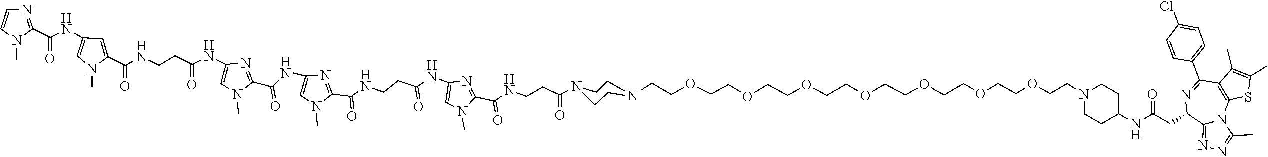 [SMILES: Cc1sc2c(c1C)C(c1ccc(Cl)cc1)=N[C@@H](CC(=O)NC1CCN(CCOCCOCCOCCOCCOCCOCCOCCN3CCN(C(=O)CCNC(=O)c4nc(NC(=O)CCNC(=O)c5nc(NC(=O)c6nc(NC(=O)CCNC(=O)c7cc(NC(=O)c8nccn8C)cn7C)cn6C)cn5C)cn4C)CC3)CC1)c1nnc(C)n1-2] | 98 |
| 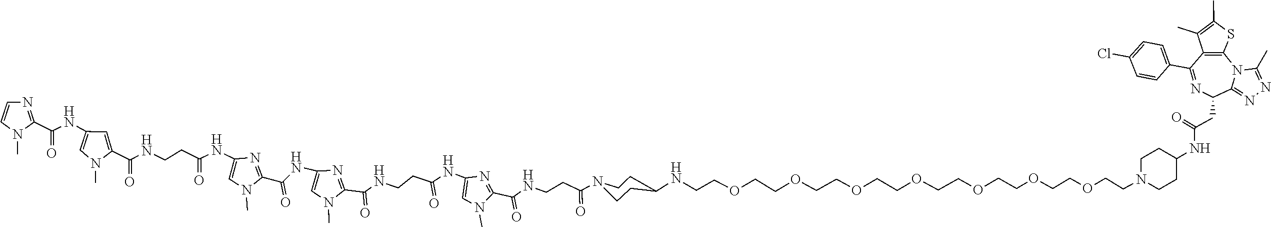 [SMILES: Cc1sc2c(c1C)C(c1ccc(Cl)cc1)=N[C@@H](CC(=O)NC1CCN(CCOCCOCCOCCOCCOCCOCCOCCNC3CCN(C(=O)CCNC(=O)c4nc(NC(=O)CCNC(=O)c5nc(NC(=O)c6nc(NC(=O)CCNC(=O)c7cc(NC(=O)c8nccn8C)cn7C)cn6C)cn5C)cn4C)CC3)CC1)c1nnc(C)n1-2] | 99 |
| 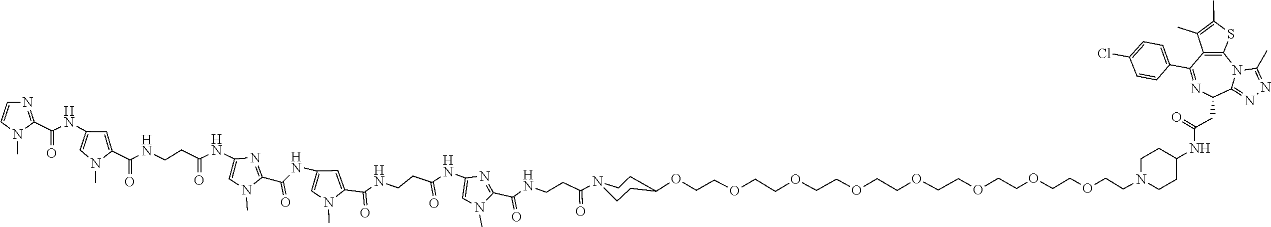 [SMILES: Cc1sc2c(c1C)C(c1ccc(Cl)cc1)=N[C@@H](CC(=O)NC1CCN(CCOCCOCCOCCOCCOCCOCCOCCOC3CCN(C(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)CC3)CC1)c1nnc(C)n1-2] | 100 |
| 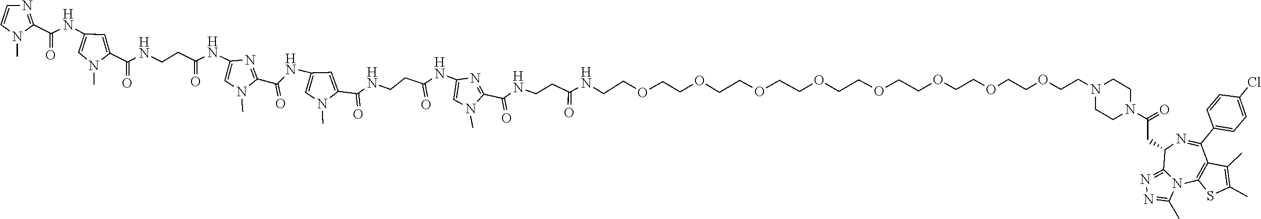 [SMILES: Cc1sc2c(c1C)C(c1ccc(Cl)cc1)=N[C@@H](CC(=O)N1CCN(CCOCCOCCOCCOCCOCCOCCOCCOCCNC(=O)CCNC(=O)c3nc(NC(=O)CCNC(=O)c4cc(NC(=O)c5nc(NC(=O)CCNC(=O)c6cc(NC(=O)c7nccn7C)cn6C)cn5C)cn4C)cn3C)CC1)c1nnc(C)n1-2] | 101 |
| 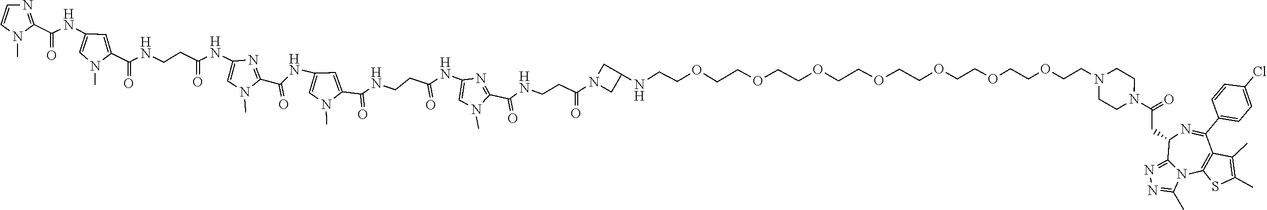 [SMILES: Cc1sc2c(c1C)C(c1ccc(Cl)cc1)=N[C@@H](CC(=O)N1CCN(CCOCCOCCOCCOCCOCCOCCOCCNC3CN(C(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)C3)CC1)c1nnc(C)n1-2] | 102 |
| 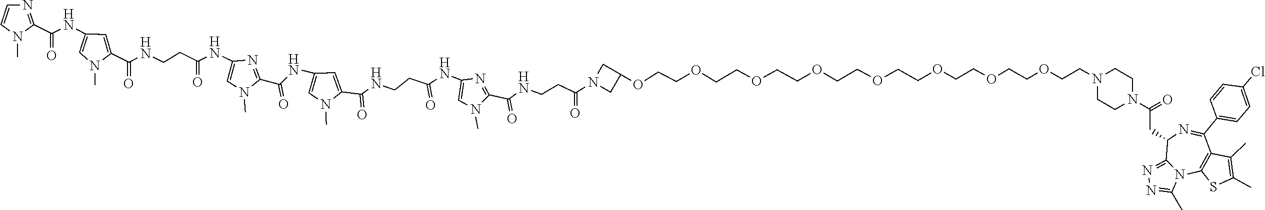 [SMILES: Cc1sc2c(c1C)C(c1ccc(Cl)cc1)=N[C@@H](CC(=O)N1CCN(CCOCCOCCOCCOCCOCCOCCOCCOC3CN(C(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)C3)CC1)c1nnc(C)n1-2] | 103 |
| 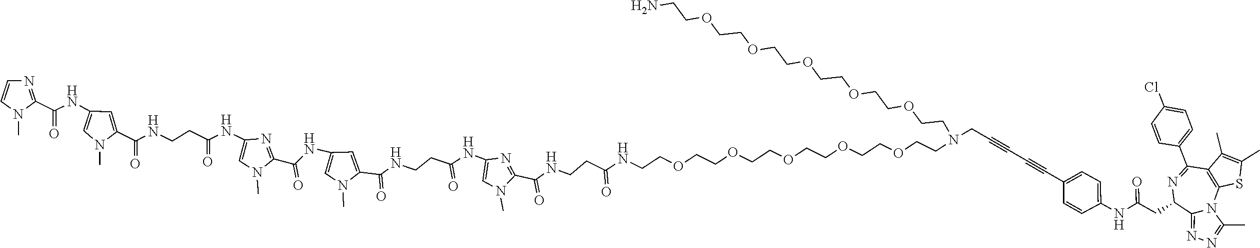 [SMILES: Cc1sc2c(c1C)C(c1ccc(Cl)cc1)=N[C@@H](CC(=O)Nc1ccc(C#CC#CCN(CCOCCOCCOCCOCCOCCN)CCOCCOCCOCCOCCOCCNC(=O)CCNC(=O)c3nc(NC(=O)CCNC(=O)c4cc(NC(=O)c5nc(NC(=O)CCNC(=O)c6cc(NC(=O)c7nccn7C)cn6C)cn5C)cn4C)cn3C)cc1)c1nnc(C)n1-2] | 104 |
| 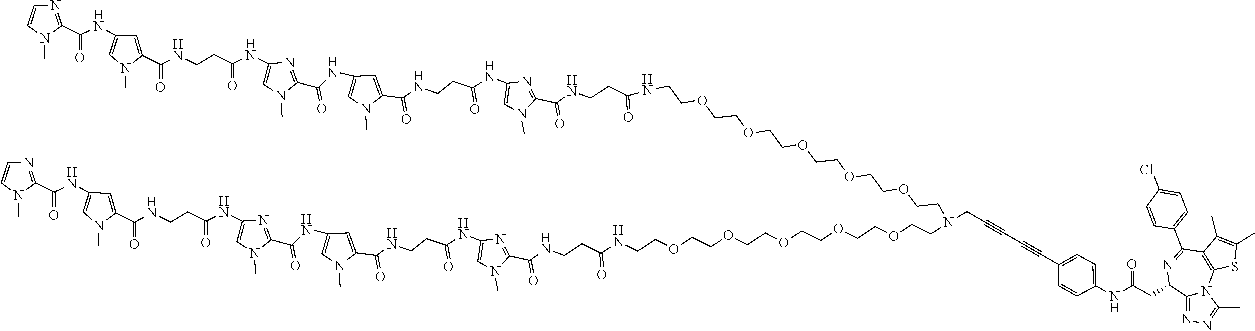 [SMILES: Cc1sc2c(c1C)C(c1ccc(Cl)cc1)=N[C@@H](CC(=O)Nc1ccc(C#CC#CCN(CCOCCOCCOCCOCCOCCNC(=O)CCNC(=O)c3nc(NC(=O)CCNC(=O)c4cc(NC(=O)c5nc(NC(=O)CCNC(=O)c6cc(NC(=O)c7nccn7C)cn6C)cn5C)cn4C)cn3C)CCOCCOCCOCCOCCOCCNC(=O)CCNC(=O)c3nc(NC(=O)CCNC(=O)c4cc(NC(=O)c5nc(NC(=O)CCNC(=O)c6cc(NC(=O)c7nccn7C)cn6C)cn5C)cn4C)cn3C)cc1)c1nnc(C)n1-2] | 105 |
| 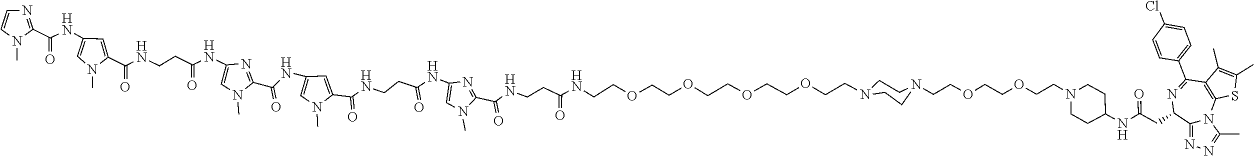 [SMILES: Cc1sc2c(c1C)C(c1ccc(Cl)cc1)=N[C@@H](CC(=O)NC1CCN(CCOCCOCCN3CCN(CCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)CC3)CC1)c1nnc(C)n1-2] | 106 |
| 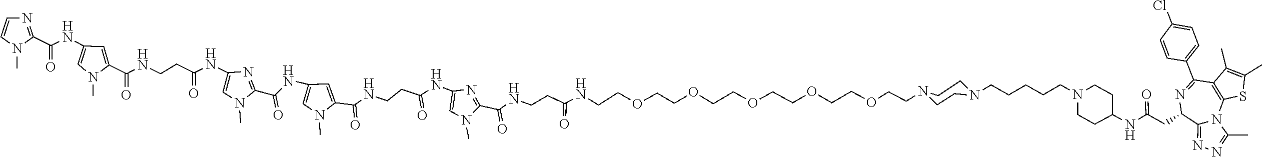 [SMILES: Cc1sc2c(c1C)C(c1ccc(Cl)cc1)=N[C@@H](CC(=O)NC1CCN(CCCCCN3CCN(CCOCCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)CC3)CC1)c1nnc(C)n1-2] | 107 |
| 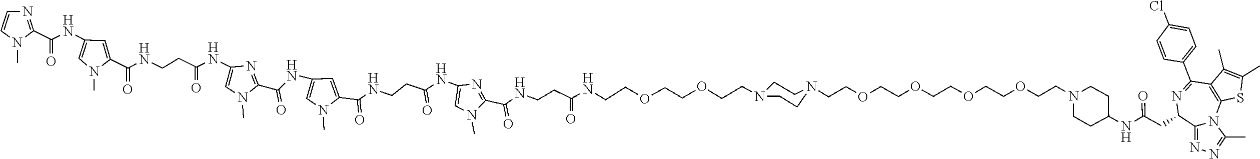 [SMILES: Cc1sc2c(c1C)C(c1ccc(Cl)cc1)=N[C@@H](CC(=O)NC1CCN(CCOCCOCCOCCOCCN3CCN(CCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)CC3)CC1)c1nnc(C)n1-2] | 108 |
| 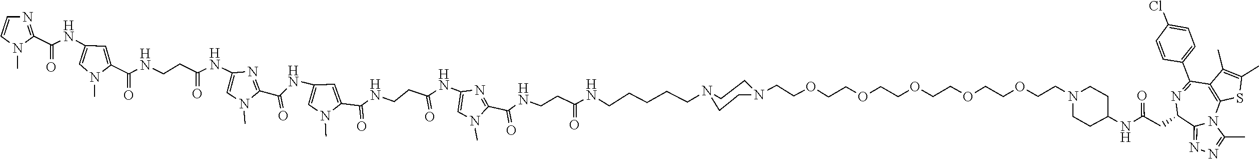 [SMILES: Cc1sc2c(c1C)C(c1ccc(Cl)cc1)=N[C@@H](CC(=O)NC1CCN(CCOCCOCCOCCOCCOCCN3CCN(CCCCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)CC3)CC1)c1nnc(C)n1-2] | 109 |
| 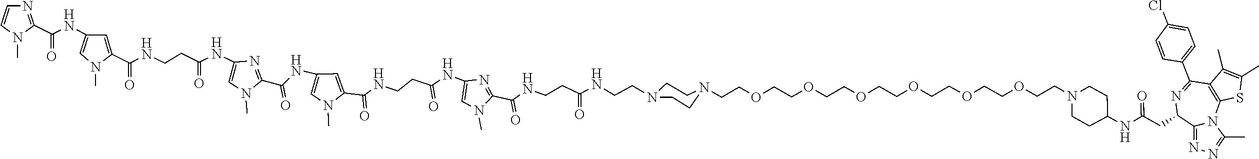 [SMILES: Cc1sc2c(c1C)C(c1ccc(Cl)cc1)=N[C@@H](CC(=O)NC1CCN(CCOCCOCCOCCOCCOCCOCCN3CCN(CCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)CC3)CC1)c1nnc(C)n1-2] | 110 |
| 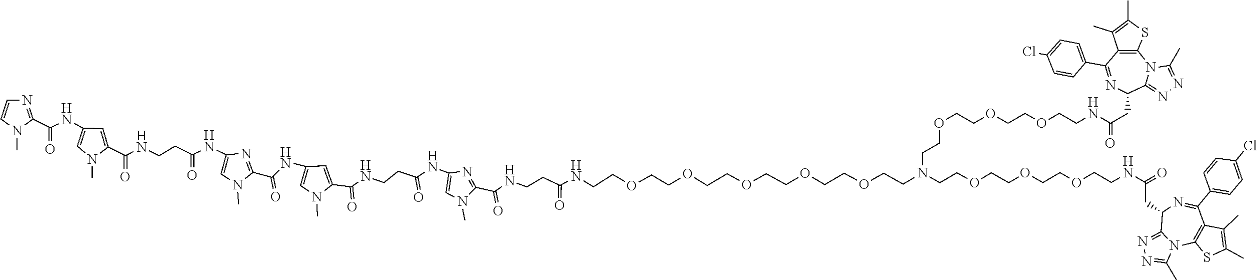 [SMILES: Cc1sc2c(c1C)C(c1ccc(Cl)cc1)=N[C@@H](CC(=O)NCCOCCOCCOCCN(CCOCCOCCOCCNC(=O)C[C@@H]1N=C(c3ccc(Cl)cc3)c3c(sc(C)c3C)-n3c(C)nnc31)CCOCCOCCOCCOCCOCCNC(=O)CCNC(=O)c1nc(NC(=O)CCNC(=O)c3cc(NC(=O)c4nc(NC(=O)CCNC(=O)c5cc(NC(=O)c6nccn6C)cn5C)cn4C)cn3C)cn1C)c1nnc(C)n1-2] | 111 |
| 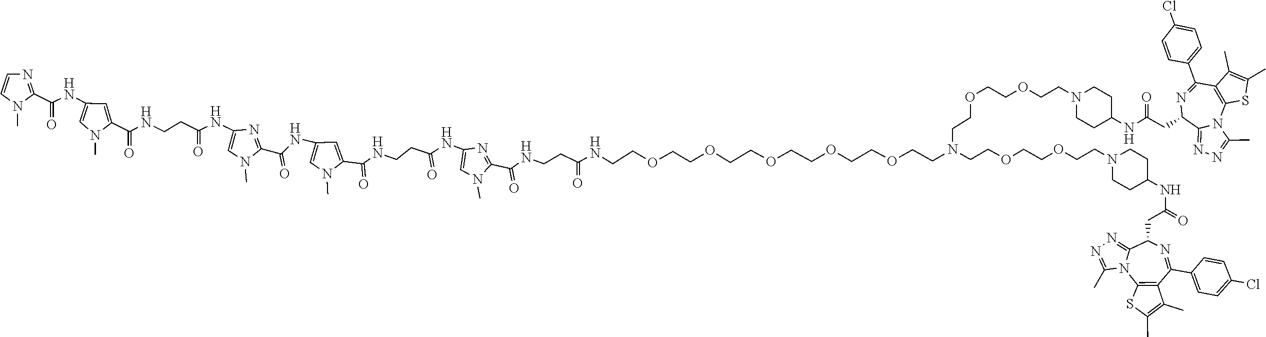 [SMILES: Cc1sc2c(c1C)C(c1ccc(Cl)cc1)=N[C@@H](CC(=O)NC1CCN(CCOCCOCCN(CCOCCOCCOCCOCCOCCNC(=O)CCNC(=O)c3nc(NC(=O)CCNC(=O)c4cc(NC(=O)c5nc(NC(=O)CCNC(=O)c6cc(NC(=O)c7nccn7C)cn6C)cn5C)cn4C)cn3C)CCOCCOCCN3CCC(NC(=O)C[C@@H]4N=C(c5ccc(Cl)cc5)c5c(sc(C)c5C)-n5c(C)nnc54)CC3)CC1)c1nnc(C)n1-2] | 112 |
| 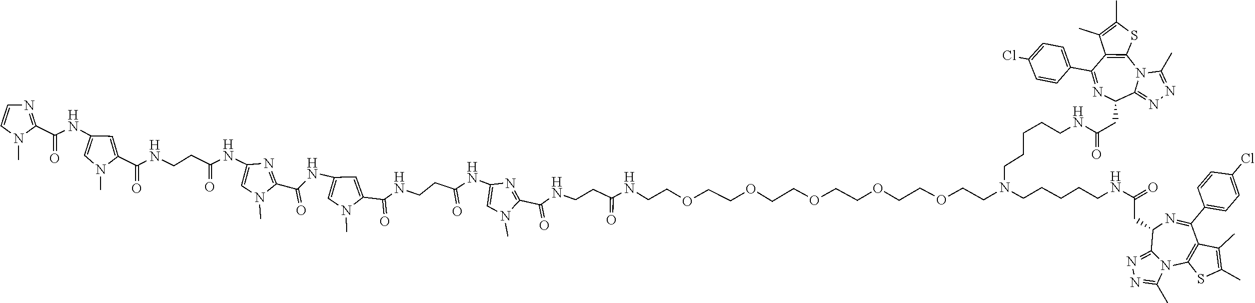 [SMILES: Cc1sc2c(c1C)C(c1ccc(Cl)cc1)=N[C@@H](CC(=O)NCCCCCN(CCCCCNC(=O)C[C@@H]1N=C(c3ccc(Cl)cc3)c3c(sc(C)c3C)-n3c(C)nnc31)CCOCCOCCOCCOCCOCCNC(=O)CCNC(=O)c1nc(NC(=O)CCNC(=O)c3cc(NC(=O)c4nc(NC(=O)CCNC(=O)c5cc(NC(=O)c6nccn6C)cn5C)cn4C)cn3C)cn1C)c1nnc(C)n1-2] | 113 |
| 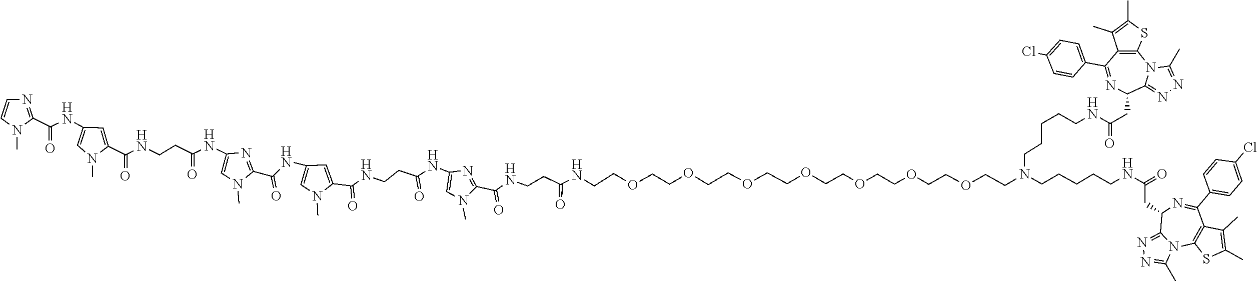 [SMILES: Cc1sc2c(c1C)C(c1ccc(Cl)cc1)=N[C@@H](CC(=O)NCCCCCN(CCCCCNC(=O)C[C@@H]1N=C(c3ccc(Cl)cc3)c3c(sc(C)c3C)-n3c(C)nnc31)CCOCCOCCOCCOCCOCCOCCOCCNC(=O)CCNC(=O)c1nc(NC(=O)CCNC(=O)c3cc(NC(=O)c4nc(NC(=O)CCNC(=O)c5cc(NC(=O)c6nccn6C)cn5C)cn4C)cn3C)cn1C)c1nnc(C)n1-2] | 114 |
| 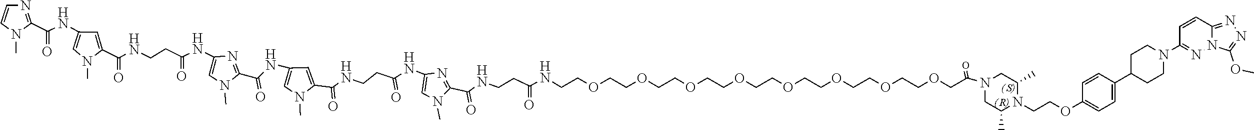 | 115 |
| 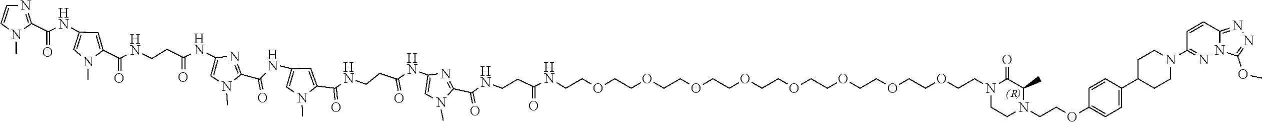 | 116 |
| 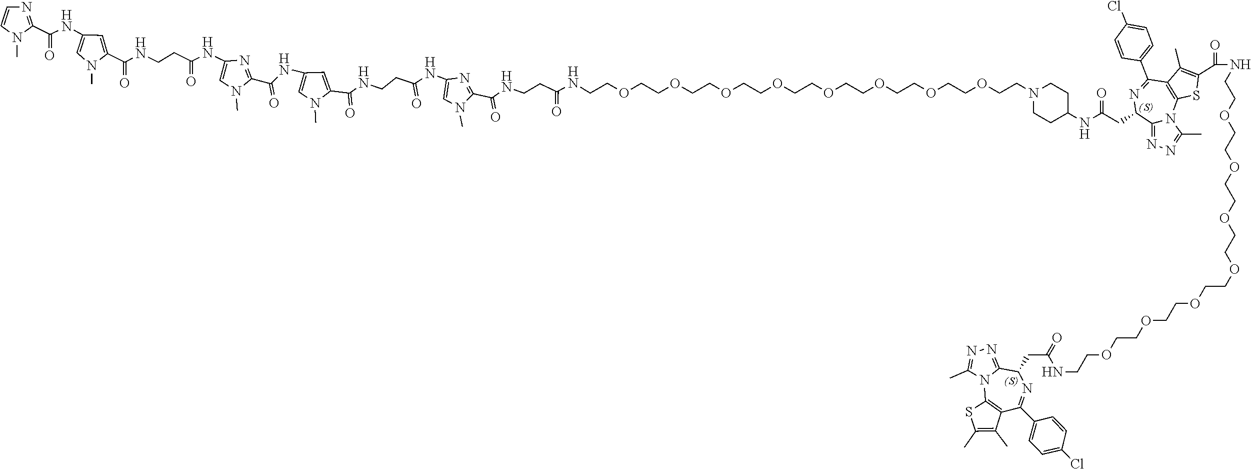 | 117 |
| 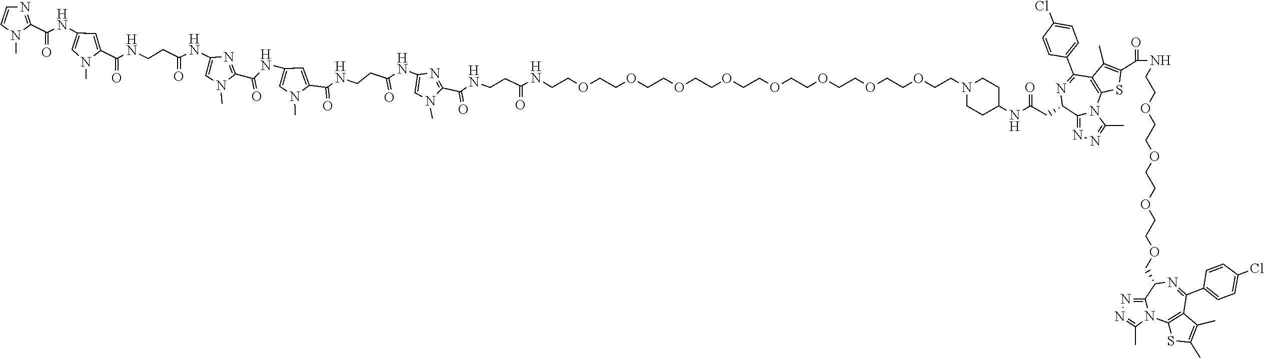 [SMILES: Cc1sc2c(c1C)C(c1ccc(Cl)cc1)=N[C@@H](COCCOCCOCCOCCNC(=O)c1sc3c(c1C)C(c1ccc(Cl)cc1)=N[C@@H](CC(=O)NC1CCN(CCOCCOCCOCCOCCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)CC1)c1nnc(C)n1-3)c1nnc(C)n1-2] | 118 |
| 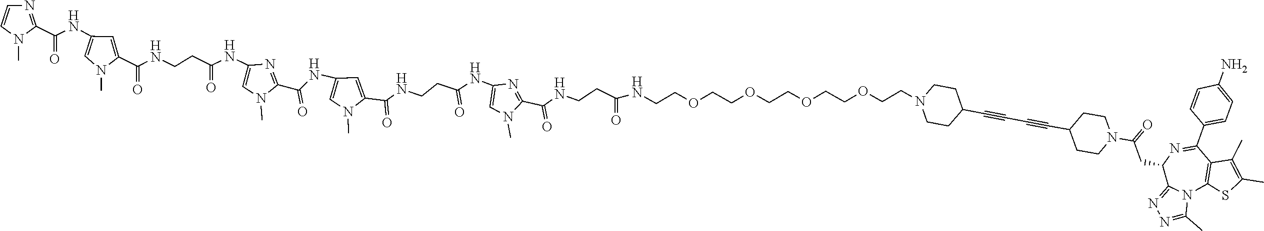 [SMILES: Cc1sc2c(c1C)C(c1ccc(N)cc1)=N[C@@H](CC(=O)N1CCC(C#CC#CC3CCN(CCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)CC3)CC1)c1nnc(C)n1-2] | 119 |
| 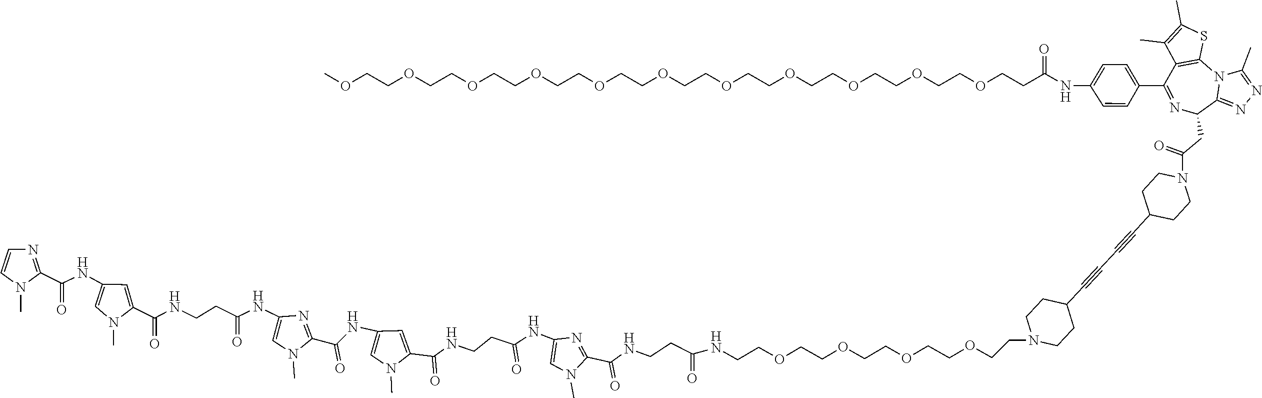 [SMILES: COCCOCCOCCOCCOCCOCCOCCOCCOCCOCCOCCC(=O)Nc1ccc(C2=N[C@@H](CC(=O)N3CCC(C#CC#CC4CCN(CCOCCOCCOCCOCCNC(=O)CCNC(=O)c5nc(NC(=O)CCNC(=O)c6cc(NC(=O)c7nc(NC(=O)CCNC(=O)c8cc(NC(=O)c9nccn9C)cn8C)cn7C)cn6C)cn5C)CC4)CC3)c3nnc(C)n3-c3sc(C)c(C)c32)cc1] | 120 |
| 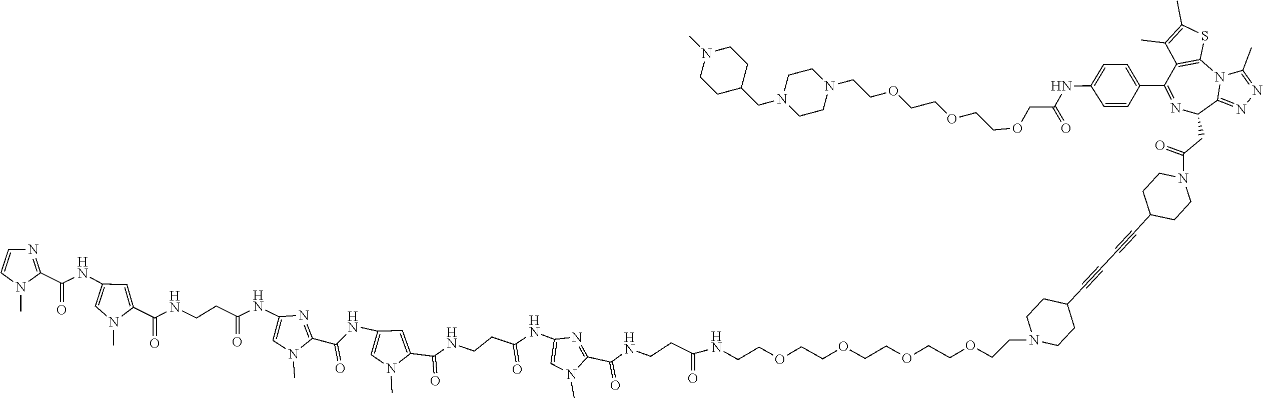 [SMILES: Cc1sc2c(c1C)C(c1ccc(NC(=O)COCCOCCOCCN3CCN(CC4CCN(C)CC4)CC3)cc1)=N[C@@H](CC(=O)N1CCC(C#CC#CC3CCN(CCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)CC3)CC1)c1nnc(C)n1-2] | 121 |
| 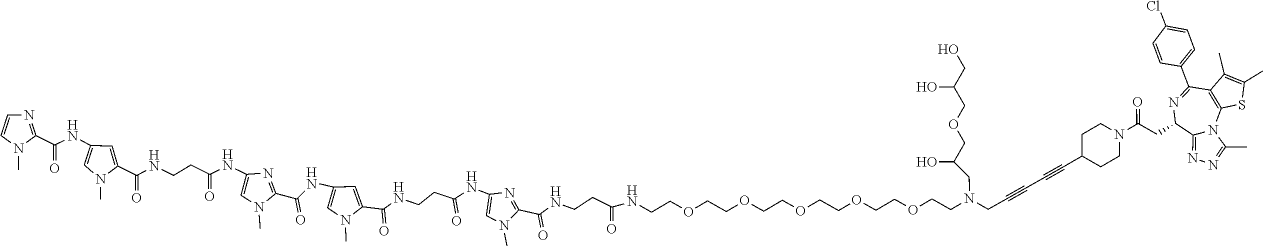 [SMILES: Cc1sc2c(c1C)C(c1ccc(Cl)cc1)=N[C@@H](CC(=O)N1CCC(C#CC#CCN(CCOCCOCCOCCOCCOCCNC(=O)CCNC(=O)c3nc(NC(=O)CCNC(=O)c4cc(NC(=O)c5nc(NC(=O)CCNC(=O)c6cc(NC(=O)c7nccn7C)cn6C)cn5C)cn4C)cn3C)CC(O)COCC(O)CO)CC1)c1nnc(C)n1-2] | 122 |
| 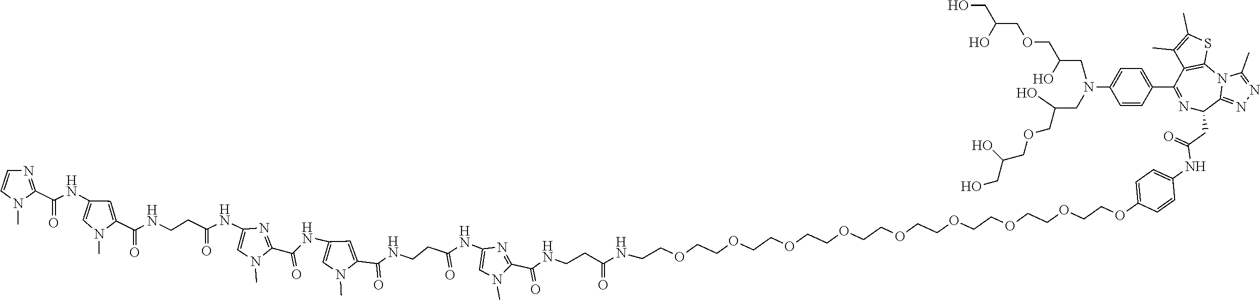 [SMILES: Cc1sc2c(c1C)C(c1ccc(N(CC(O)COCC(O)CO)CC(O)COCC(O)CO)cc1)=N[C@@H](CC(=O)Nc1ccc(OCCOCCOCCOCCOCCOCCOCCOCCOCCNC(=O)CCNC(=O)c3nc(NC(=O)CCNC(=O)c4cc(NC(=O)c5nc(NC(=O)CCNC(=O)c6cc(NC(=O)c7nccn7C)cn6C)cn5C)cn4C)cn3C)cc1)c1nnc(C)n1-2] | 123 |
| 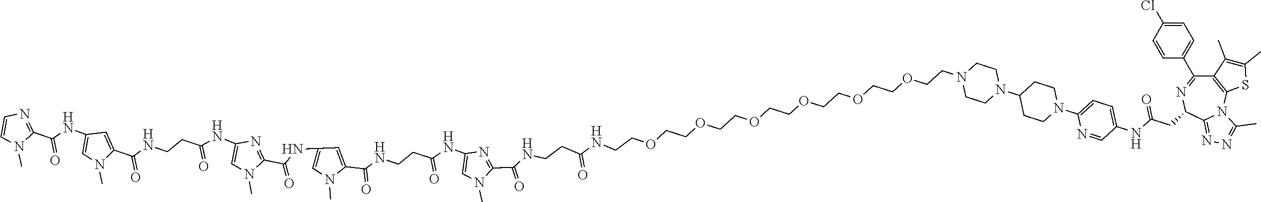 [SMILES: Cc1sc2c(c1C)C(c1ccc(Cl)cc1)=N[C@@H](CC(=O)Nc1ccc(N3CCC(N4CCN(CCOCCOCCOCCOCCOCCOCCNC(=O)CCNC(=O)c5nc(NC(=O)CCNC(=O)c6cc(NC(=O)c7nc(NC(=O)CCNC(=O)c8cc(NC(=O)c9nccn9C)cn8C)cn7C)cn6C)cn5C)CC4)CC3)nc1)c1nnc(C)n1-2] | 124 |
| 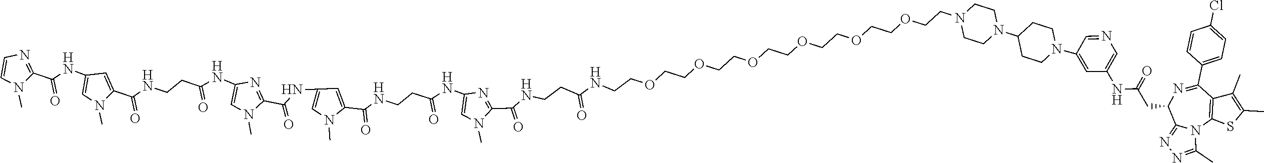 [SMILES: Cc1sc2c(c1C)C(c1ccc(Cl)cc1)=N[C@@H](CC(=O)Nc1cncc(N3CCC(N4CCN(CCOCCOCCOCCOCCOCCOCCNC(=O)CCNC(=O)c5nc(NC(=O)CCNC(=O)c6cc(NC(=O)c7nc(NC(=O)CCNC(=O)c8cc(NC(=O)c9nccn9C)cn8C)cn7C)cn6C)cn5C)CC4)CC3)c1)c1nnc(C)n1-2] | 125 |
| 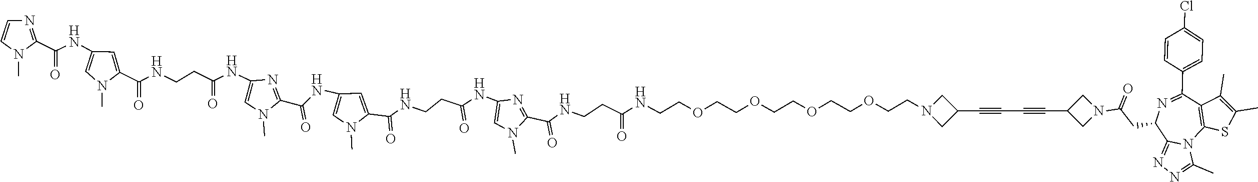 [SMILES: Cc1sc2c(c1C)C(c1ccc(Cl)cc1)=N[C@@H](CC(=O)N1CC(C#CC#CC3CN(CCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)C3)C1)c1nnc(C)n1-2] | 126 |
| 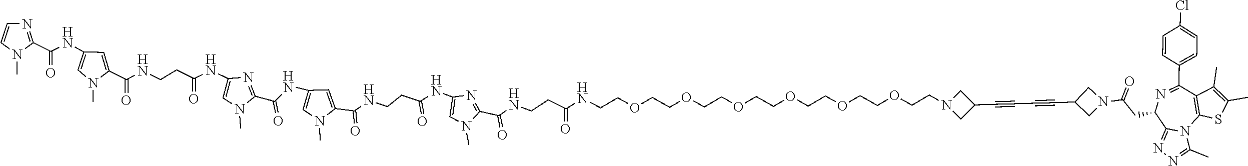 [SMILES: Cc1sc2c(c1C)C(c1ccc(Cl)cc1)=N[C@@H](CC(=O)N1CC(C#CC#CC3CN(CCOCCOCCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)C3)C1)c1nnc(C)n1-2] | 127 |
| 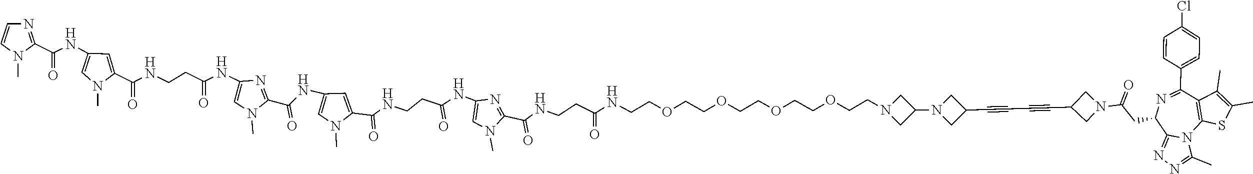 [SMILES: Cc1sc2c(c1C)C(c1ccc(Cl)cc1)=N[C@@H](CC(=O)N1CC(C#CC#CC3CN(C4CN(CCOCCOCCOCCOCCNC(=O)CCNC(=O)c5nc(NC(=O)CCNC(=O)c6cc(NC(=O)c7nc(NC(=O)CCNC(=O)c8cc(NC(=O)c9nccn9C)cn8C)cn7C)cn6C)cn5C)C4)C3)C1)c1nnc(C)n1-2] | 128 |
| 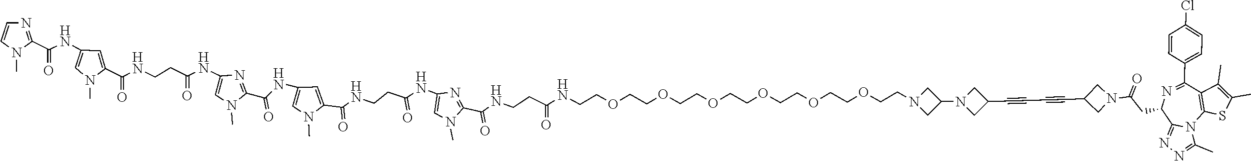 [SMILES: Cc1sc2c(c1C)C(c1ccc(Cl)cc1)=N[C@@H](CC(=O)N1CC(C#CC#CC3CN(C4CN(CCOCCOCCOCCOCCOCCOCCNC(=O)CCNC(=O)c5nc(NC(=O)CCNC(=O)c6cc(NC(=O)c7nc(NC(=O)CCNC(=O)c8cc(NC(=O)c9nccn9C)cn8C)cn7C)cn6C)cn5C)C4)C3)C1)c1nnc(C)n1-2] | 129 |
| 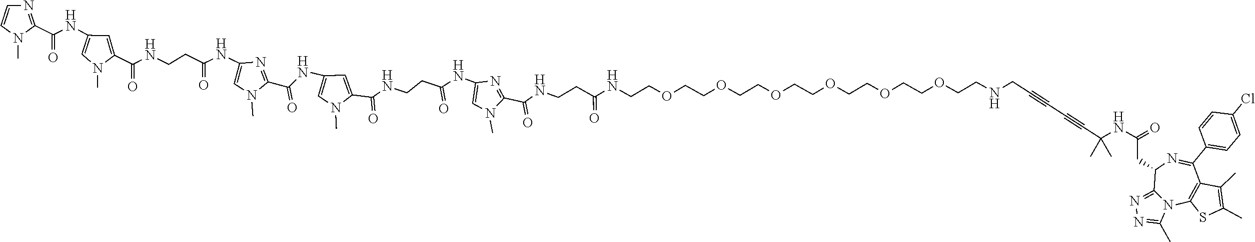 [SMILES: Cc1sc2c(c1C)C(c1ccc(Cl)cc1)=N[C@@H](CC(=O)NC(C)(C)C#CC#CCNCCOCCOCCOCCOCCOCCOCCNC(=O)CCNC(=O)c1nc(NC(=O)CCNC(=O)c3cc(NC(=O)c4nc(NC(=O)CCNC(=O)c5cc(NC(=O)c6nccn6C)cn5C)cn4C)cn3C)cn1C)c1nnc(C)n1-2] | 130 |
| 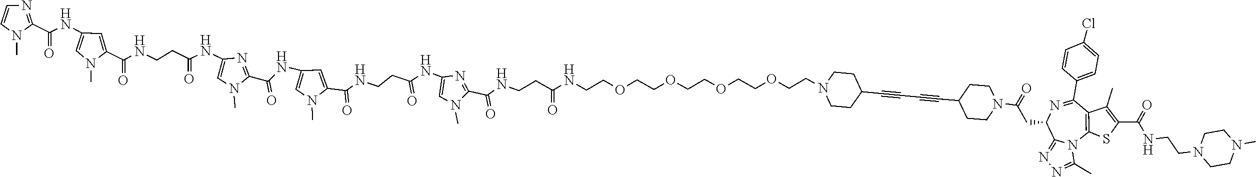 [SMILES: Cc1c(C(=O)NCCN2CCN(C)CC2)sc2c1C(c1ccc(Cl)cc1)=N[C@@H](CC(=O)N1CCC(C#CC#CC3CCN(CCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)CC3)CC1)c1nnc(C)n1-2] | 131 |
| 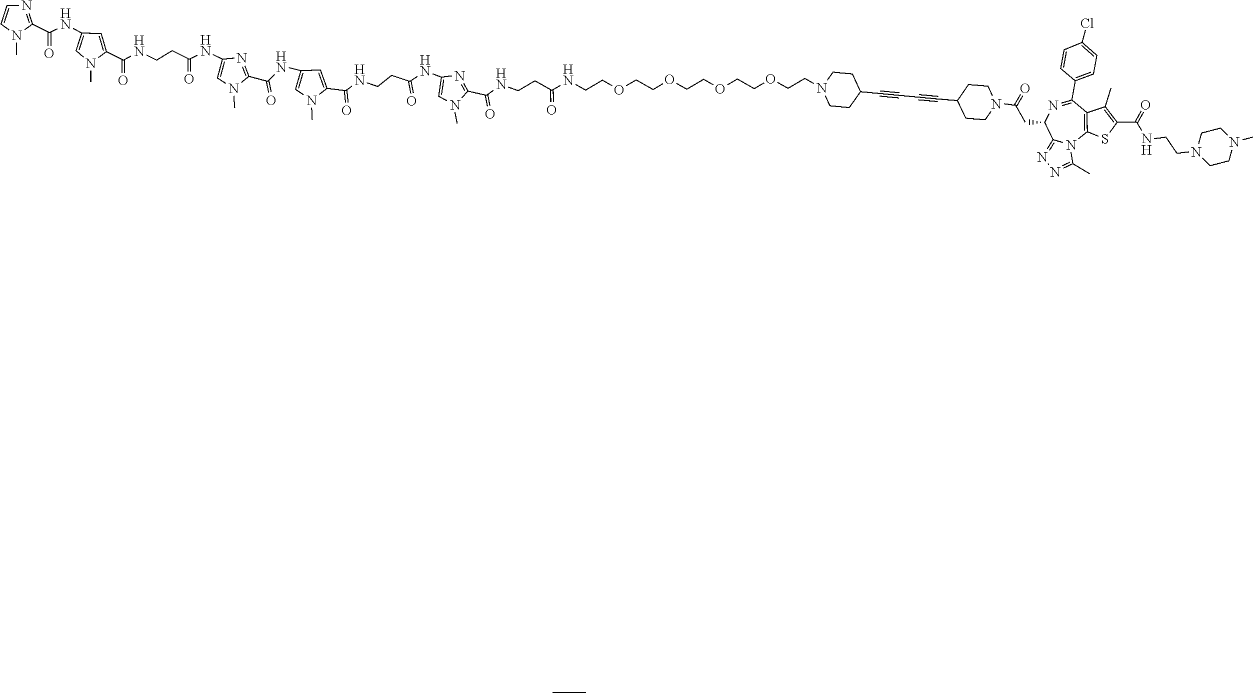 | 132 |
| 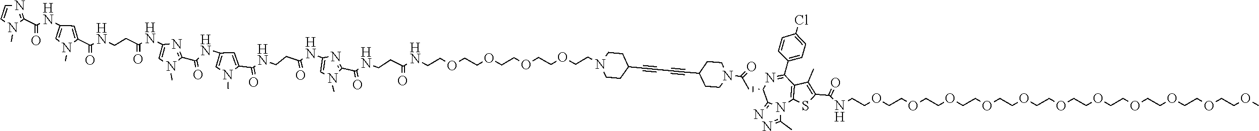 [SMILES: COCCOCCOCCOCCOCCOCCOCCOCCOCCOCCOCCNC(=O)c1sc2c(c1C)C(c1ccc(Cl)cc1)=N[C@@H](CC(=O)N1CCC(C#CC#CC3CCN(CCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)CC3)CC1)c1nnc(C)n1-2] | 133 |
| 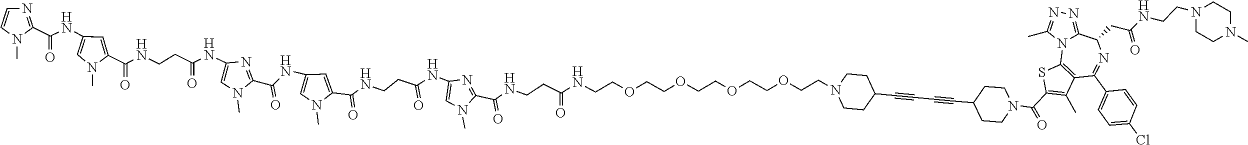 [SMILES: Cc1c(C(=O)N2CCC(C#CC#CC3CCN(CCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)CC3)CC2)sc2c1C(c1ccc(Cl)cc1)=N[C@@H](CC(=O)NCCN1CCN(C)CC1)c1nnc(C)n1-2] | 134 |
| 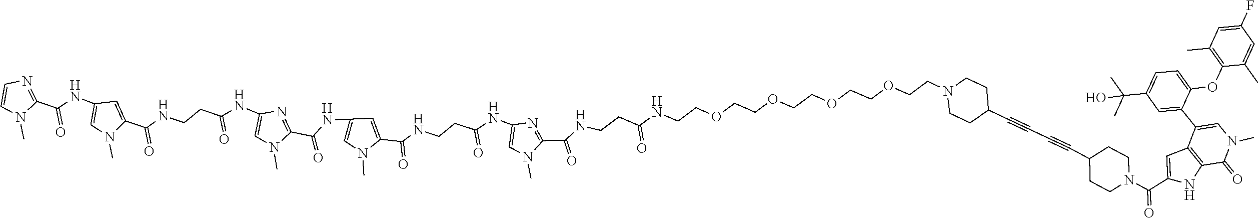 [SMILES: Cc1cc(F)cc(C)c1Oc1ccc(C(C)(C)O)cc1-c1cn(C)c(=O)c2[nH]c(C(=O)N3CCC(C#CC#CC4CCN(CCOCCOCCOCCOCCNC(=O)CCNC(=O)c5nc(NC(=O)CCNC(=O)c6cc(NC(=O)c7nc(NC(=O)CCNC(=O)c8cc(NC(=O)c9nccn9C)cn8C)cn7C)cn6C)cn5C)CC4)CC3)cc12] | 135 |
| 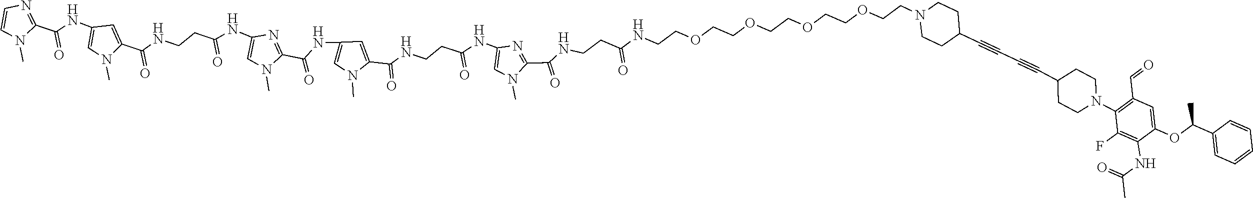 [SMILES: CC(=O)Nc1c(O[C@@H](C)c2ccccc2)cc(C=O)c(N2CCC(C#CC#CC3CCN(CCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)CC3)CC2)c1F] | 136 |
| 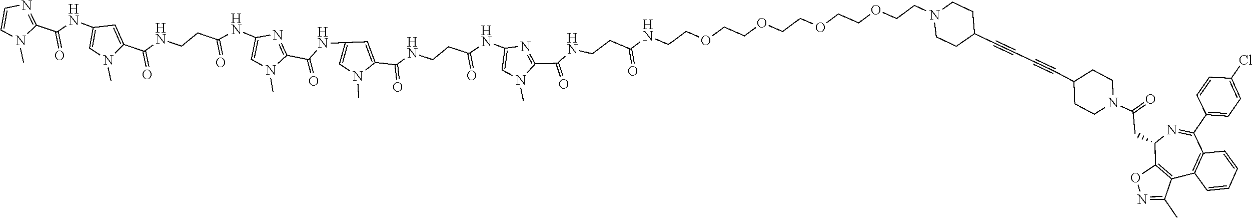 [SMILES: Cc1noc2c1-c1ccccc1C(c1ccc(Cl)cc1)=N[C@H]2CC(=O)N1CCC(C#CC#CC2CCN(CCOCCOCCOCCOCCNC(=O)CCNC(=O)c3nc(NC(=O)CCNC(=O)c4cc(NC(=O)c5nc(NC(=O)CCNC(=O)c6cc(NC(=O)c7nccn7C)cn6C)cn5C)cn4C)cn3C)CC2)CC1] | 137 |
| 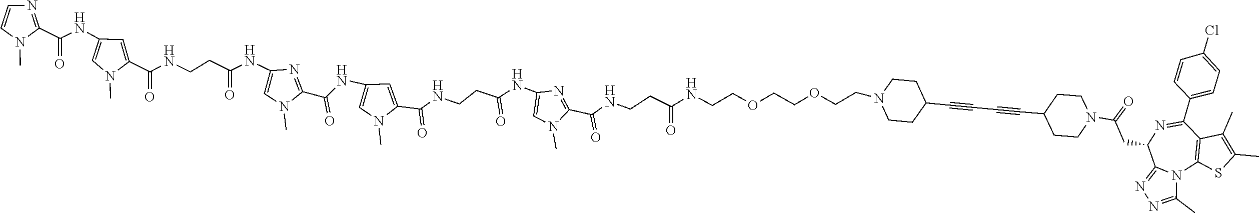 [SMILES: Cc1sc2c(c1C)C(c1ccc(Cl)cc1)=N[C@@H](CC(=O)N1CCC(C#CC#CC3CCN(CCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)CC3)CC1)c1nnc(C)n1-2] | 138 |
| 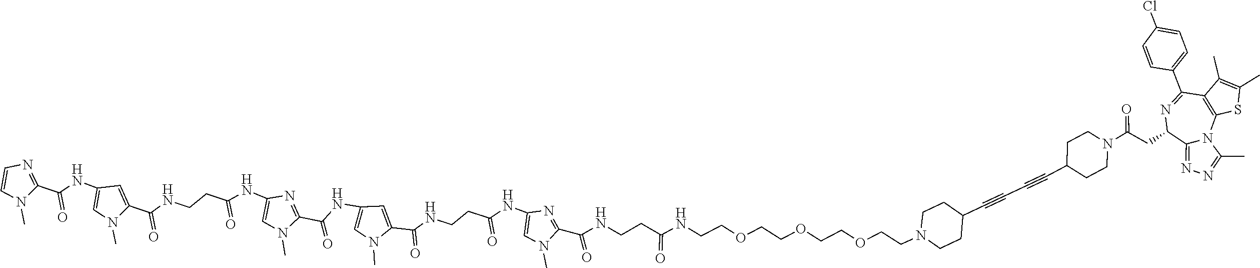 [SMILES: Cc1sc2c(c1C)C(c1ccc(Cl)cc1)=N[C@@H](CC(=O)N1CCC(C#CC#CC3CCN(CCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)CC3)CC1)c1nnc(C)n1-2] | 139 |
| 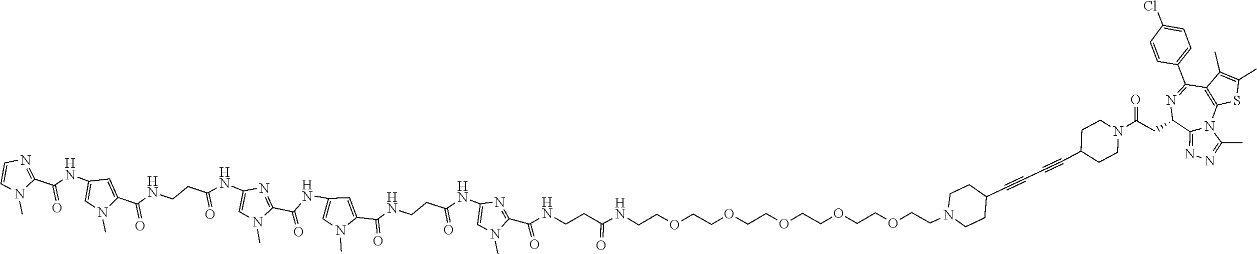 [SMILES: Cc1sc2c(c1C)C(c1ccc(Cl)cc1)=N[C@@H](CC(=O)N1CCC(C#CC#CC3CCN(CCOCCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)CC3)CC1)c1nnc(C)n1-2] | 140 |
| 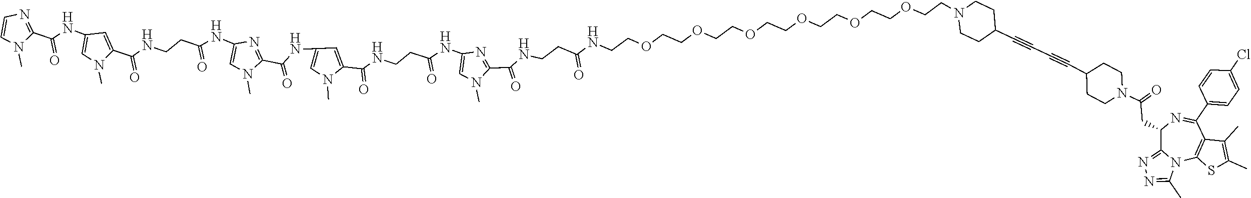 [SMILES: Cc1sc2c(c1C)C(c1ccc(Cl)cc1)=N[C@@H](CC(=O)N1CCC(C#CC#CC3CCN(CCOCCOCCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)CC3)CC1)c1nnc(C)n1-2] | 141 |
| 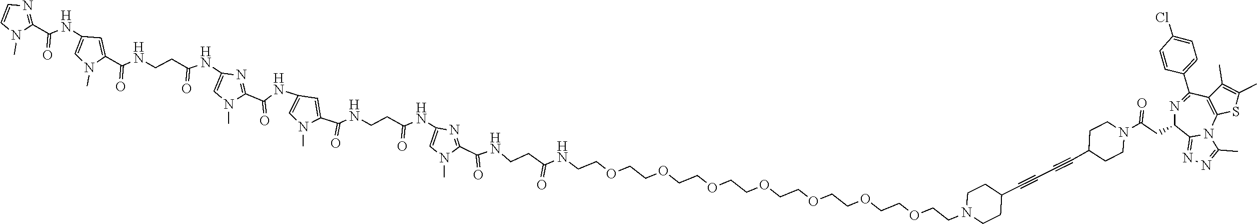 [SMILES: Cc1sc2c(c1C)C(c1ccc(Cl)cc1)=N[C@@H](CC(=O)N1CCC(C#CC#CC3CCN(CCOCCOCCOCCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)CC3)CC1)c1nnc(C)n1-2] | 142 |
| 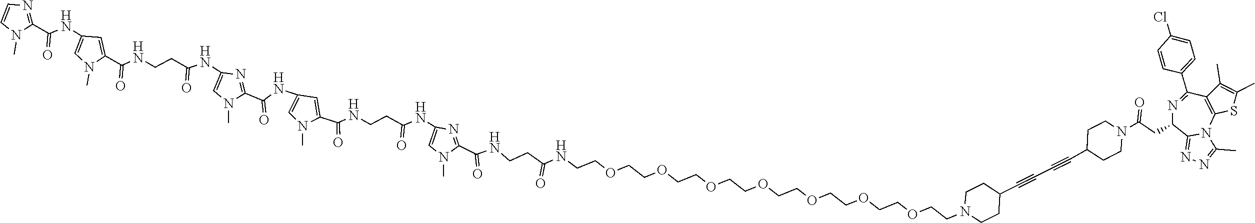 [SMILES: Cc1sc2c(c1C)C(c1ccc(Cl)cc1)=N[C@@H](CC(=O)N1CCC(C#CC#CC3CCN(CCOCCOCCOCCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)CC3)CC1)c1nnc(C)n1-2] | 143 |
| 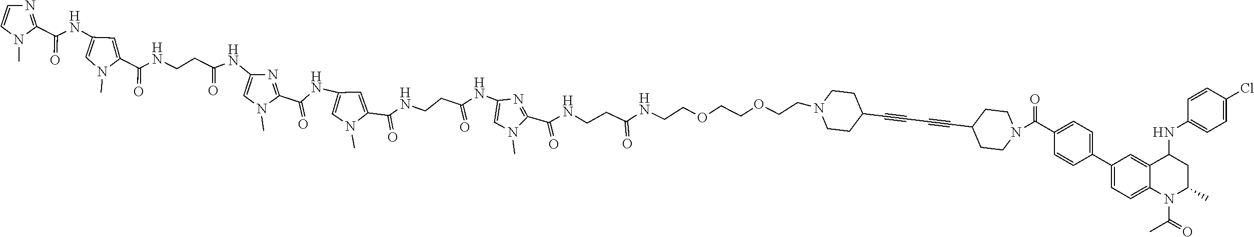 [SMILES: CC(=O)N1c2ccc(-c3ccc(C(=O)N4CCC(C#CC#CC5CCN(CCOCCOCCNC(=O)CCNC(=O)c6nc(NC(=O)CCNC(=O)c7cc(NC(=O)c8nc(NC(=O)CCNC(=O)c9cc(NC(=O)c%10nccn%10C)cn9C)cn8C)cn7C)cn6C)CC5)CC4)cc3)cc2C(Nc2ccc(Cl)cc2)C[C@@H]1C] | 144 |
| 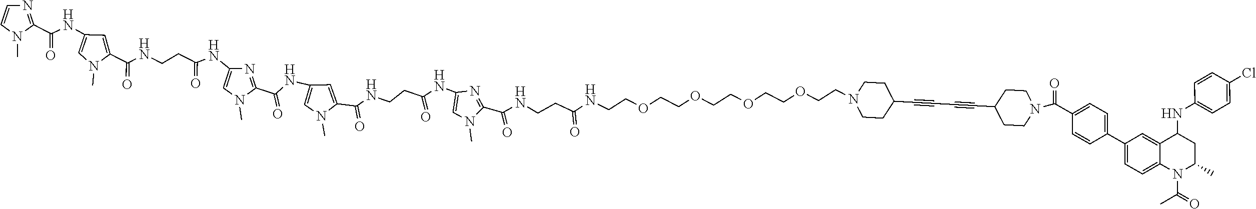 [SMILES: CC(=O)N1c2ccc(-c3ccc(C(=O)N4CCC(C#CC#CC5CCN(CCOCCOCCOCCOCCNC(=O)CCNC(=O)c6nc(NC(=O)CCNC(=O)c7cc(NC(=O)c8nc(NC(=O)CCNC(=O)c9cc(NC(=O)c%10nccn%10C)cn9C)cn8C)cn7C)cn6C)CC5)CC4)cc3)cc2C(Nc2ccc(Cl)cc2)C[C@@H]1C] | 145 |
| 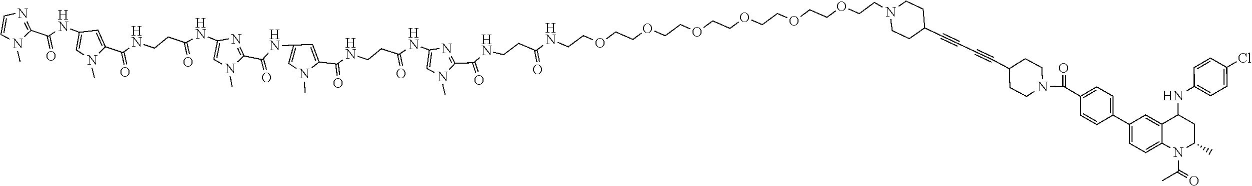 [SMILES: CC(=O)N1c2ccc(-c3ccc(C(=O)N4CCC(C#CC#CC5CCN(CCOCCOCCOCCOCCOCCOCCNC(=O)CCNC(=O)c6nc(NC(=O)CCNC(=O)c7cc(NC(=O)c8nc(NC(=O)CCNC(=O)c9cc(NC(=O)c%10nccn%10C)cn9C)cn8C)cn7C)cn6C)CC5)CC4)cc3)cc2C(Nc2ccc(Cl)cc2)C[C@@H]1C] | 150 |
| 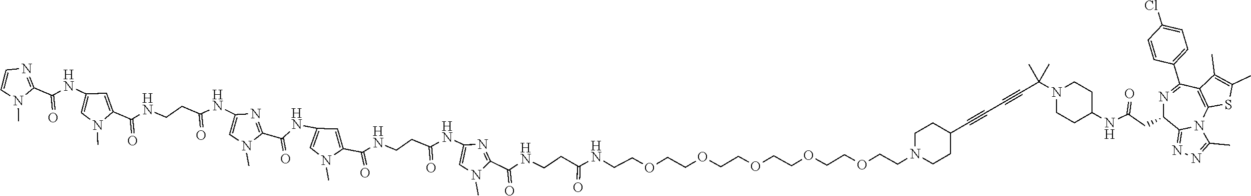 [SMILES: Cc1sc2c(c1C)C(c1ccc(Cl)cc1)=N[C@@H](CC(=O)NC1CCN(C(C)(C)C#CC#CC3CCN(CCOCCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)CC3)CC1)c1nnc(C)n1-2] | 151 |
| 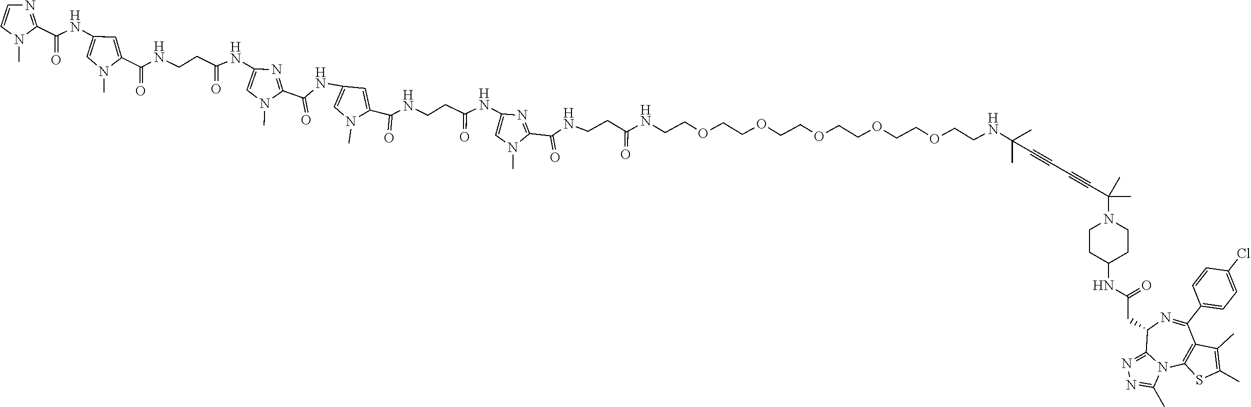 [SMILES: Cc1sc2c(c1C)C(c1ccc(Cl)cc1)=N[C@@H](CC(=O)NC1CCN(C(C)(C)C#CC#CC(C)(C)NCCOCCOCCOCCOCCOCCNC(=O)CCNC(=O)c3nc(NC(=O)CCNC(=O)c4cc(NC(=O)c5nc(NC(=O)CCNC(=O)c6cc(NC(=O)c7nccn7C)cn6C)cn5C)cn4C)cn3C)CC1)c1nnc(C)n1-2] | 152 |
| 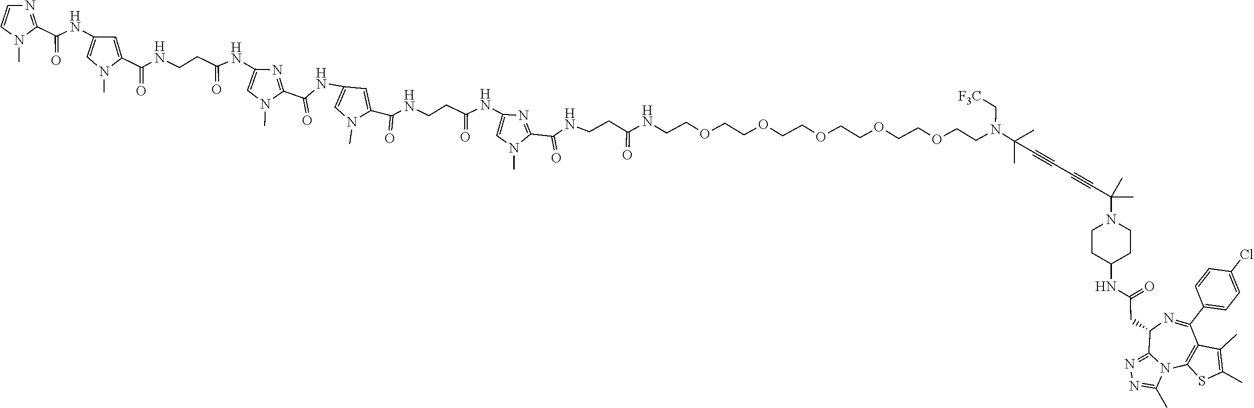 [SMILES: Cc1sc2c(c1C)C(c1ccc(Cl)cc1)=N[C@@H](CC(=O)NC1CCN(C(C)(C)C#CC#CC(C)(C)N(CCOCCOCCOCCOCCOCCNC(=O)CCNC(=O)c3nc(NC(=O)CCNC(=O)c4cc(NC(=O)c5nc(NC(=O)CCNC(=O)c6cc(NC(=O)c7nccn7C)cn6C)cn5C)cn4C)cn3C)CC(F)(F)F)CC1)c1nnc(C)n1-2] | 153 |
| 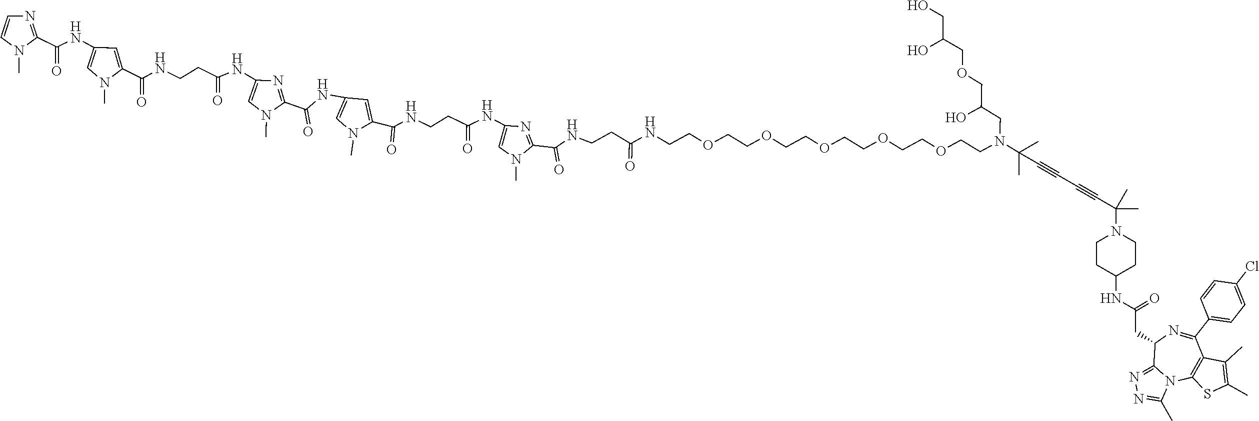 [SMILES: Cc1sc2c(c1C)C(c1ccc(Cl)cc1)=N[C@@H](CC(=O)NC1CCN(C(C)(C)C#CC#CC(C)(C)N(CCOCCOCCOCCOCCOCCNC(=O)CCNC(=O)c3nc(NC(=O)CCNC(=O)c4cc(NC(=O)c5nc(NC(=O)CCNC(=O)c6cc(NC(=O)c7nccn7C)cn6C)cn5C)cn4C)cn3C)CC(O)COCC(O)CO)CC1)c1nnc(C)n1-2] | 154 |
| 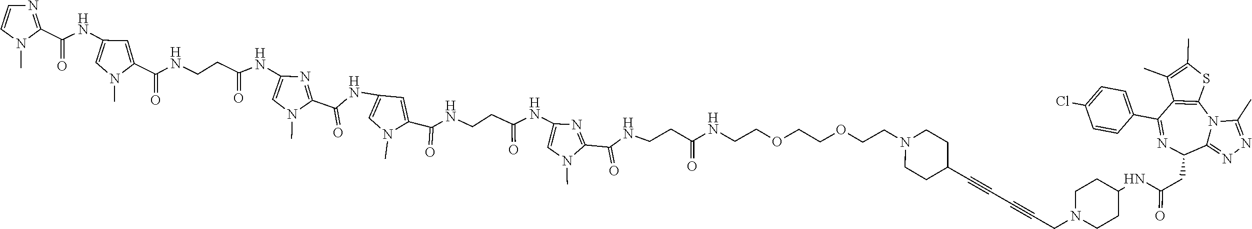 [SMILES: Cc1sc2c(c1C)C(c1ccc(Cl)cc1)=N[C@@H](CC(=O)NC1CCN(CC#CC#CC3CCN(CCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)CC3)CC1)c1nnc(C)n1-2] | 155 |
| 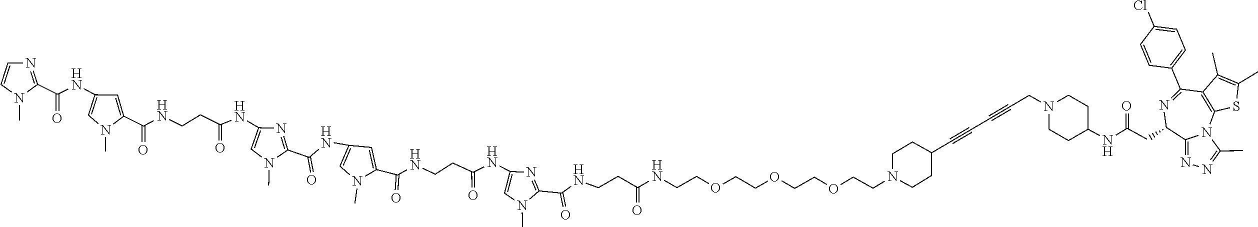 [SMILES: Cc1sc2c(c1C)C(c1ccc(Cl)cc1)=N[C@@H](CC(=O)NC1CCN(CC#CC#CC3CCN(CCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)CC3)CC1)c1nnc(C)n1-2] | 156 |
| 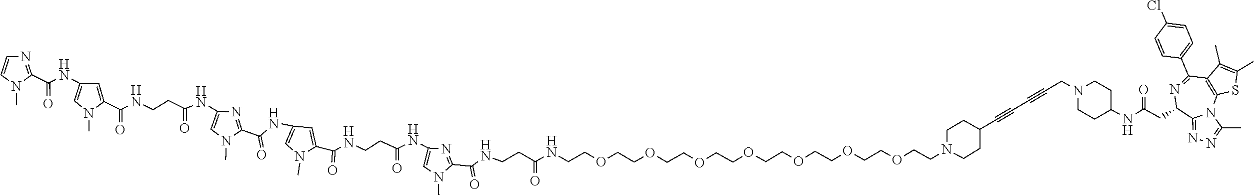 [SMILES: Cc1sc2c(c1C)C(c1ccc(Cl)cc1)=N[C@@H](CC(=O)NC1CCN(CC#CC#CC3CCN(CCOCCOCCOCCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)CC3)CC1)c1nnc(C)n1-2] | 157 |
| 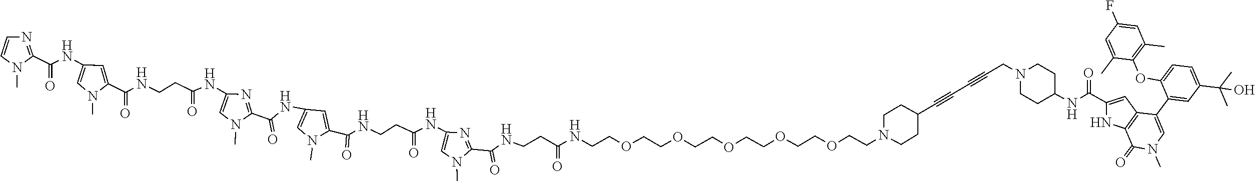 [SMILES: Cc1cc(F)cc(C)c1Oc1ccc(C(C)(C)O)cc1-c1cn(C)c(=O)c2[nH]c(C(=O)NC3CCN(CC#CC#CC4CCN(CCOCCOCCOCCOCCOCCNC(=O)CCNC(=O)c5nc(NC(=O)CCNC(=O)c6cc(NC(=O)c7nc(NC(=O)CCNC(=O)c8cc(NC(=O)c9nccn9C)cn8C)cn7C)cn6C)cn5C)CC4)CC3)cc12] | 158 |
| 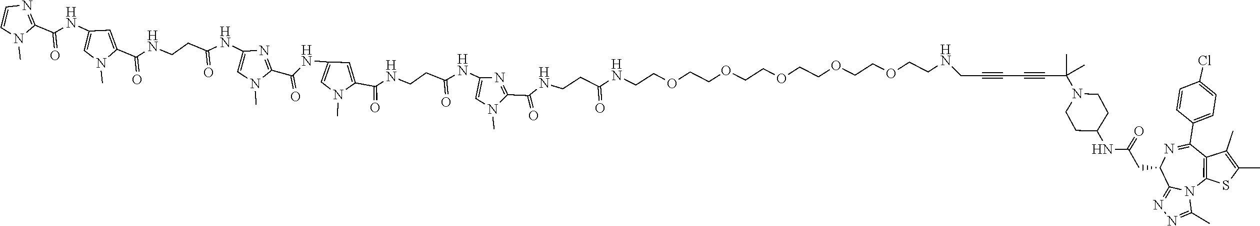 [SMILES: Cc1sc2c(c1C)C(c1ccc(Cl)cc1)=N[C@@H](CC(=O)NC1CCN(C(C)(C)C#CC#CCNCCOCCOCCOCCOCCOCCNC(=O)CCNC(=O)c3nc(NC(=O)CCNC(=O)c4cc(NC(=O)c5nc(NC(=O)CCNC(=O)c6cc(NC(=O)c7nccn7C)cn6C)cn5C)cn4C)cn3C)CC1)c1nnc(C)n1-2] | 159 |
| 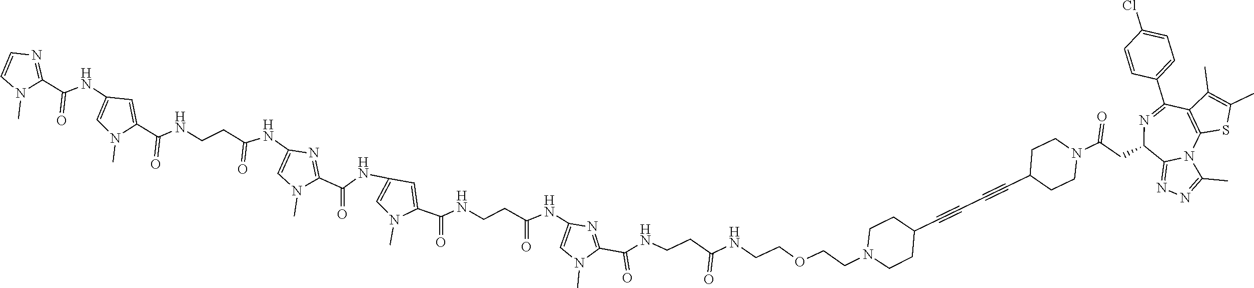 [SMILES: Cc1sc2c(c1C)C(c1ccc(Cl)cc1)=N[C@@H](CC(=O)N1CCC(C#CC#CC3CCN(CCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)CC3)CC1)c1nnc(C)n1-2] | 160 |
| 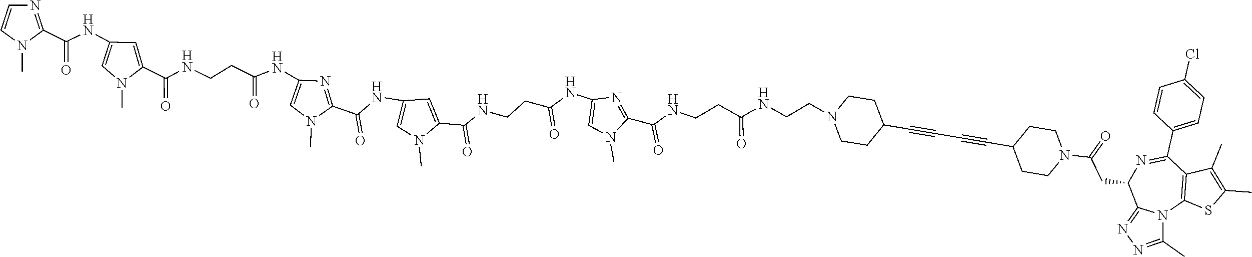 [SMILES: Cc1sc2c(c1C)C(c1ccc(Cl)cc1)=N[C@@H](CC(=O)N1CCC(C#CC#CC3CCN(CCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)CC3)CC1)c1nnc(C)n1-2] | 161 |
| 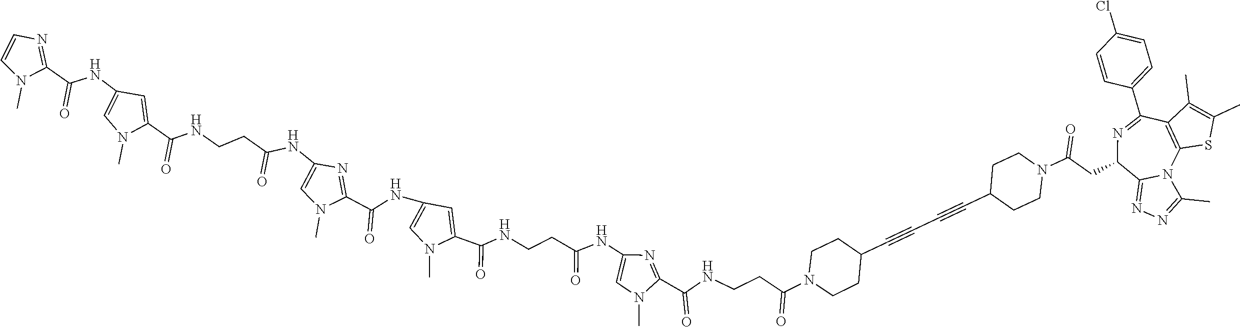 [SMILES: Cc1sc2c(c1C)C(c1ccc(Cl)cc1)=N[C@@H](CC(=O)N1CCC(C#CC#CC3CCN(C(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)CC3)CC1)c1nnc(C)n1-2] | 162 |
| 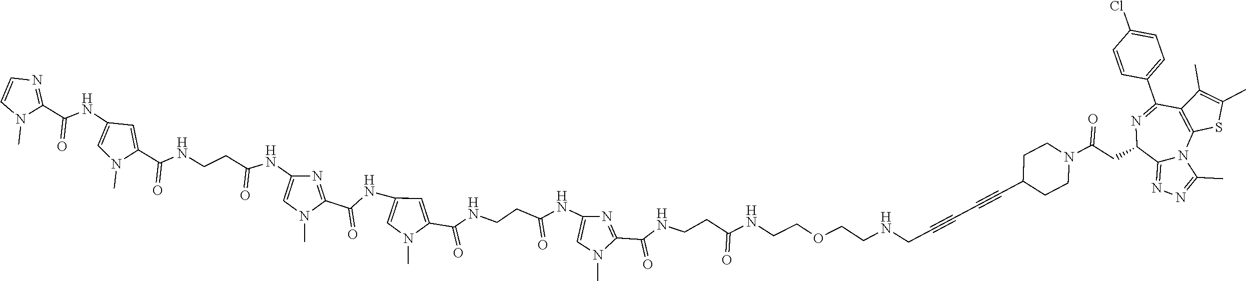 [SMILES: Cc1sc2c(c1C)C(c1ccc(Cl)cc1)=N[C@@H](CC(=O)N1CCC(C#CC#CCNCCOCCNC(=O)CCNC(=O)c3nc(NC(=O)CCNC(=O)c4cc(NC(=O)c5nc(NC(=O)CCNC(=O)c6cc(NC(=O)c7nccn7C)cn6C)cn5C)cn4C)cn3C)CC1)c1nnc(C)n1-2] | 163 |
| 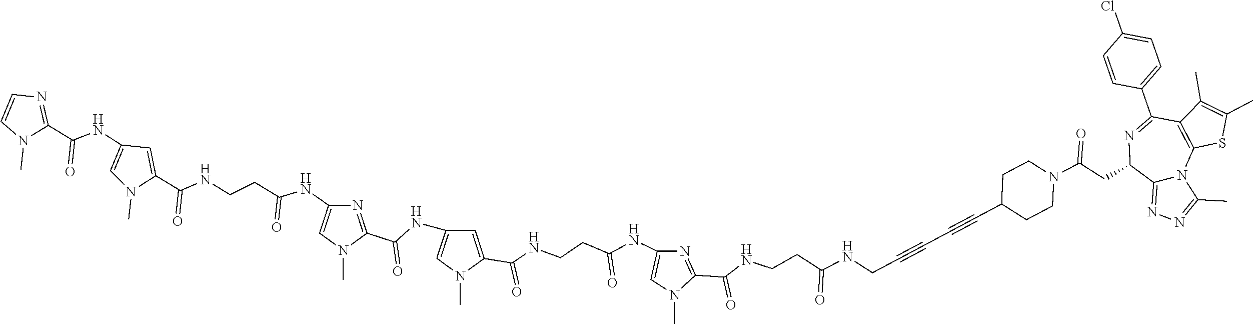 [SMILES: Cc1sc2c(c1C)C(c1ccc(Cl)cc1)=N[C@@H](CC(=O)N1CCC(C#CC#CCNC(=O)CCNC(=O)c3nc(NC(=O)CCNC(=O)c4cc(NC(=O)c5nc(NC(=O)CCNC(=O)c6cc(NC(=O)c7nccn7C)cn6C)cn5C)cn4C)cn3C)CC1)c1nnc(C)n1-2] | 164 |
| 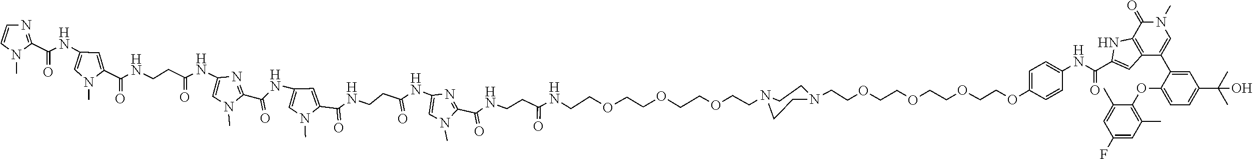 [SMILES: Cc1cc(F)cc(C)c1Oc1ccc(C(C)(C)O)cc1-c1cn(C)c(=O)c2[nH]c(C(=O)Nc3ccc(OCCOCCOCCOCCN4CCN(CCOCCOCCOCCNC(=O)CCNC(=O)c5nc(NC(=O)CCNC(=O)c6cc(NC(=O)c7nc(NC(=O)CCNC(=O)c8cc(NC(=O)c9nccn9C)cn8C)cn7C)cn6C)cn5C)CC4)cc3)cc12] | 165 |
| 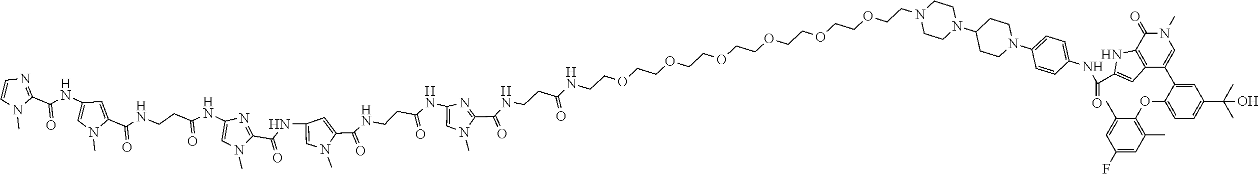 [SMILES: Cc1cc(F)cc(C)c1Oc1ccc(C(C)(C)O)cc1-c1cn(C)c(=O)c2[nH]c(C(=O)Nc3ccc(N4CCC(N5CCN(CCOCCOCCOCCOCCOCCOCCNC(=O)CCNC(=O)c6nc(NC(=O)CCNC(=O)c7cc(NC(=O)c8nc(NC(=O)CCNC(=O)c9cc(NC(=O)c%10nccn%10C)cn9C)cn8C)cn7C)cn6C)CC5)CC4)cc3)cc12] | 166 |
| 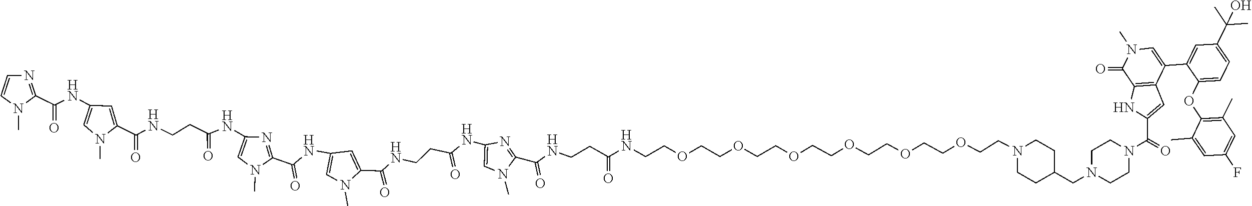 [SMILES: Cc1cc(F)cc(C)c1Oc1ccc(C(C)(C)O)cc1-c1cn(C)c(=O)c2[nH]c(C(=O)N3CCN(CC4CCN(CCOCCOCCOCCOCCOCCOCCNC(=O)CCNC(=O)c5nc(NC(=O)CCNC(=O)c6cc(NC(=O)c7nc(NC(=O)CCNC(=O)c8cc(NC(=O)c9nccn9C)cn8C)cn7C)cn6C)cn5C)CC4)CC3)cc12] | 167 |
| 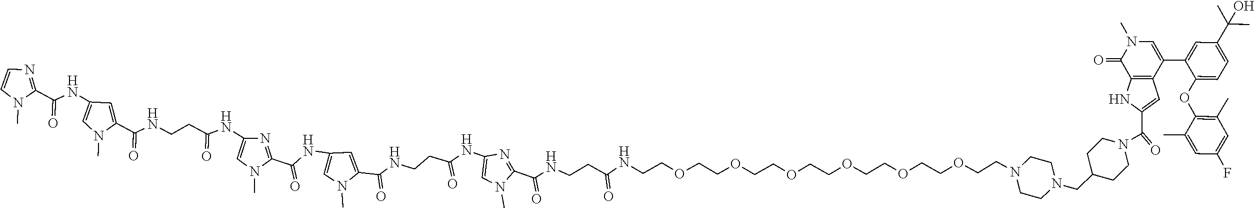 [SMILES: Cc1cc(F)cc(C)c1Oc1ccc(C(C)(C)O)cc1-c1cn(C)c(=O)c2[nH]c(C(=O)N3CCC(CN4CCN(CCOCCOCCOCCOCCOCCOCCNC(=O)CCNC(=O)c5nc(NC(=O)CCNC(=O)c6cc(NC(=O)c7nc(NC(=O)CCNC(=O)c8cc(NC(=O)c9nccn9C)cn8C)cn7C)cn6C)cn5C)CC4)CC3)cc12] | 168 |
| 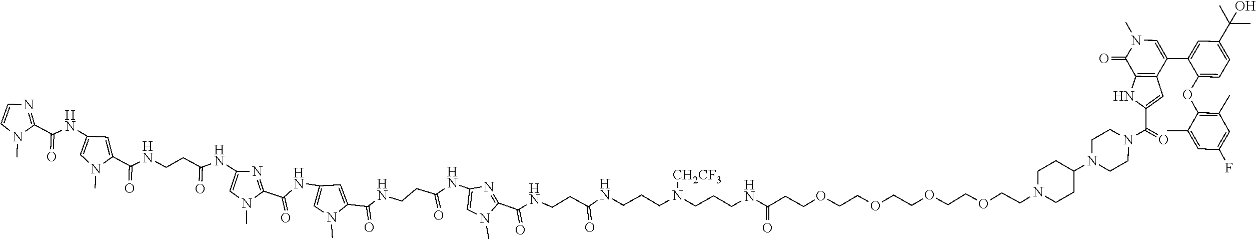 | 169 |
| 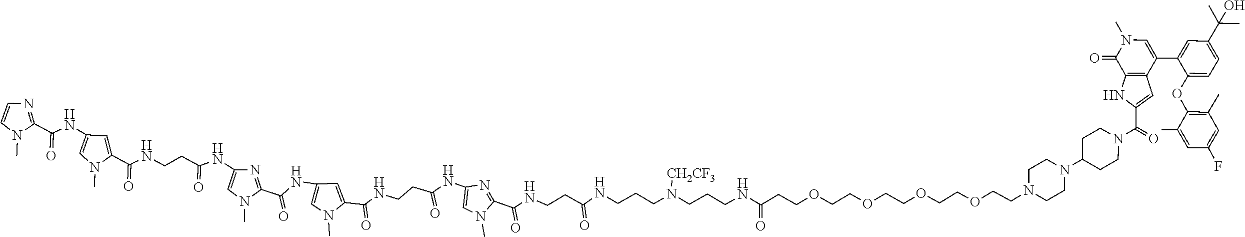 | 170 |
| 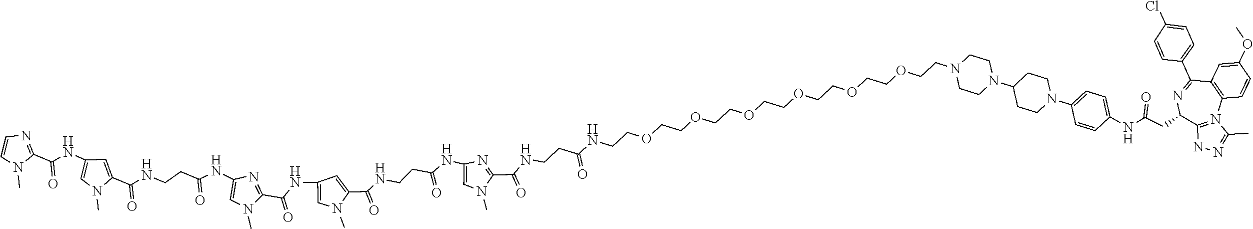 | 171 |
| 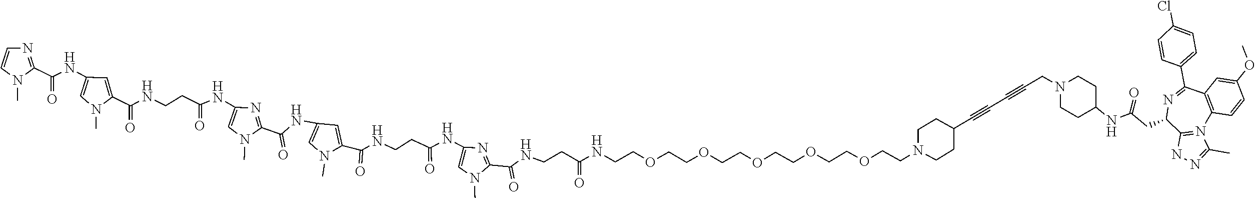 | 172 |
| 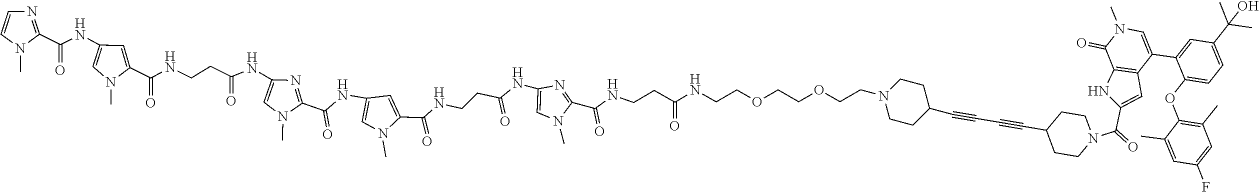 [SMILES: Cc1cc(F)cc(C)c1Oc1ccc(C(C)(C)O)cc1-c1cn(C)c(=O)c2[nH]c(C(=O)N3CCC(C#CC#CC4CCN(CCOCCOCCNC(=O)CCNC(=O)c5nc(NC(=O)CCNC(=O)c6cc(NC(=O)c7nc(NC(=O)CCNC(=O)c8cc(NC(=O)c9nccn9C)cn8C)cn7C)cn6C)cn5C)CC4)CC3)cc12] | 173 |
| 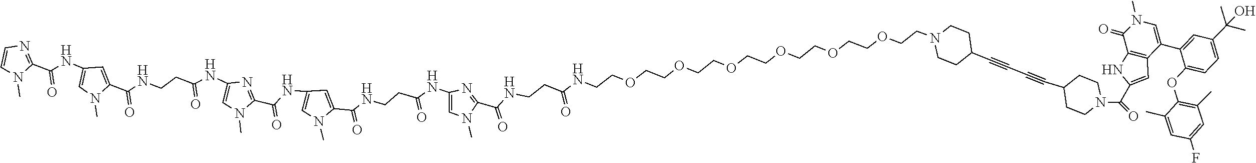 [SMILES: Cc1cc(F)cc(C)c1Oc1ccc(C(C)(C)O)cc1-c1cn(C)c(=O)c2[nH]c(C(=O)N3CCC(C#CC#CC4CCN(CCOCCOCCOCCOCCOCCOCCNC(=O)CCNC(=O)c5nc(NC(=O)CCNC(=O)c6cc(NC(=O)c7nc(NC(=O)CCNC(=O)c8cc(NC(=O)c9nccn9C)cn8C)cn7C)cn6C)cn5C)CC4)CC3)cc12] | 174 |
| 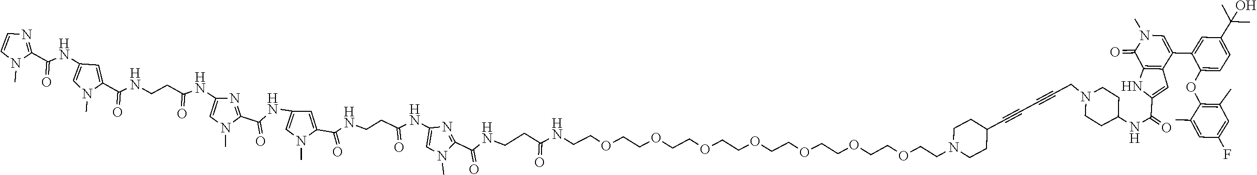 [SMILES: Cc1cc(F)cc(C)c1Oc1ccc(C(C)(C)O)cc1-c1cn(C)c(=O)c2[nH]c(C(=O)NC3CCN(CC#CC#CC4CCN(CCOCCOCCOCCOCCOCCOCCOCCNC(=O)CCNC(=O)c5nc(NC(=O)CCNC(=O)c6cc(NC(=O)c7nc(NC(=O)CCNC(=O)c8cc(NC(=O)c9nccn9C)cn8C)cn7C)cn6C)cn5C)CC4)CC3)cc12] | 175 |
| 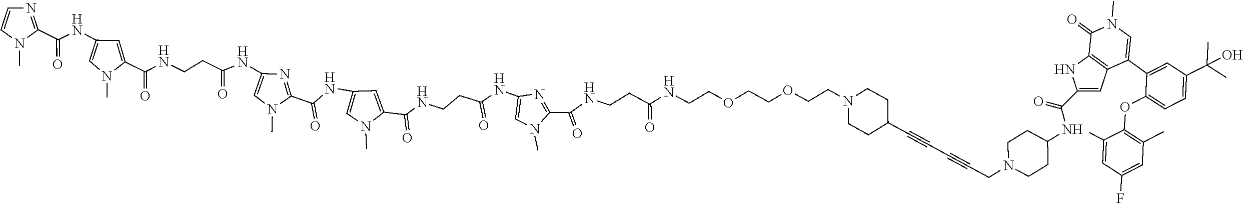 [SMILES: Cc1cc(F)cc(C)c1Oc1ccc(C(C)(C)O)cc1-c1cn(C)c(=O)c2[nH]c(C(=O)NC3CCN(CC#CC#CC4CCN(CCOCCOCCNC(=O)CCNC(=O)c5nc(NC(=O)CCNC(=O)c6cc(NC(=O)c7nc(NC(=O)CCNC(=O)c8cc(NC(=O)c9nccn9C)cn8C)cn7C)cn6C)cn5C)CC4)CC3)cc12] | 176 |
| 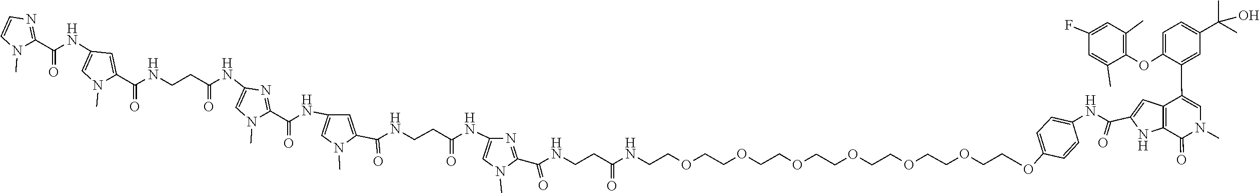 [SMILES: Cc1cc(F)cc(C)c1Oc1ccc(C(C)(C)O)cc1-c1cn(C)c(=O)c2[nH]c(C(=O)Nc3ccc(OCCOCCOCCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)cc3)cc12] | 177 |
| 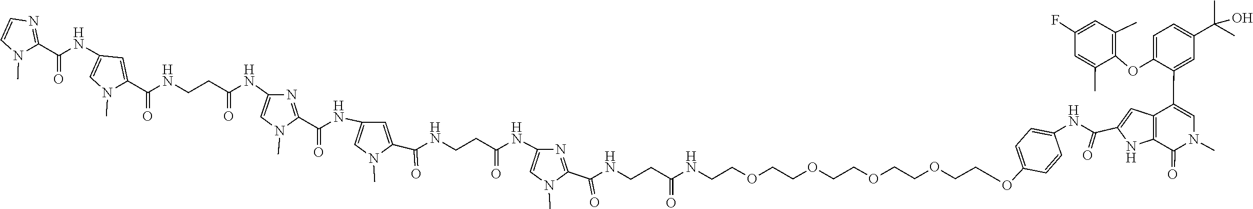 [SMILES: Cc1cc(F)cc(C)c1Oc1ccc(C(C)(C)O)cc1-c1cn(C)c(=O)c2[nH]c(C(=O)Nc3ccc(OCCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)cc3)cc12] | 178 |
| 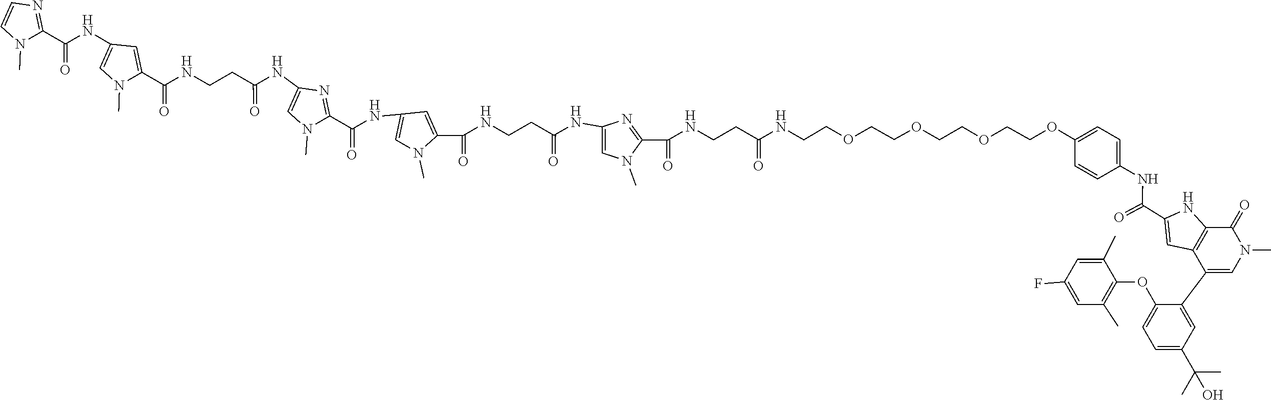 [SMILES: Cc1cc(F)cc(C)c1Oc1ccc(C(C)(C)O)cc1-c1cn(C)c(=O)c2[nH]c(C(=O)Nc3ccc(OCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)cc3)cc12] | 179 |
| 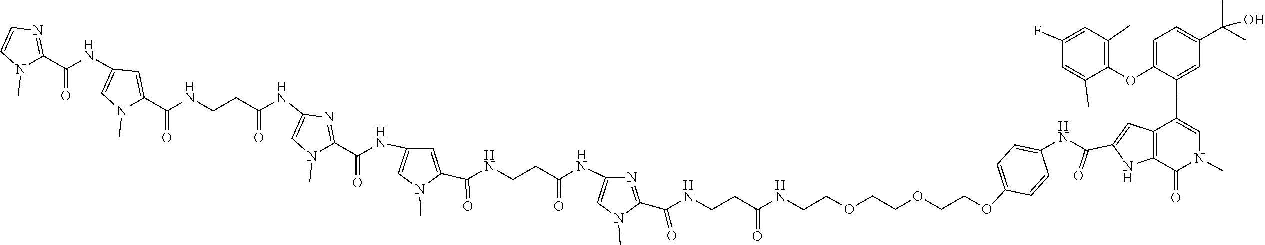 [SMILES: Cc1cc(F)cc(C)c1Oc1ccc(C(C)(C)O)cc1-c1cn(C)c(=O)c2[nH]c(C(=O)Nc3ccc(OCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)cc3)cc12] | 180 |
| 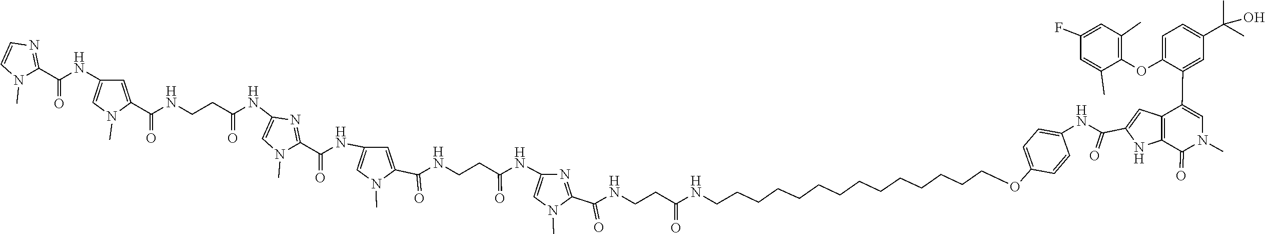 [SMILES: Cc1cc(F)cc(C)c1Oc1ccc(C(C)(C)O)cc1-c1cn(C)c(=O)c2[nH]c(C(=O)Nc3ccc(OCCCCCCCCCCCCCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)cc3)cc12] | 181 |
| 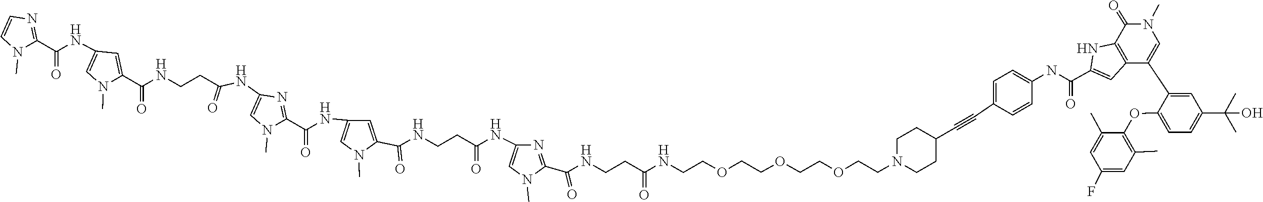 | 182 |
| 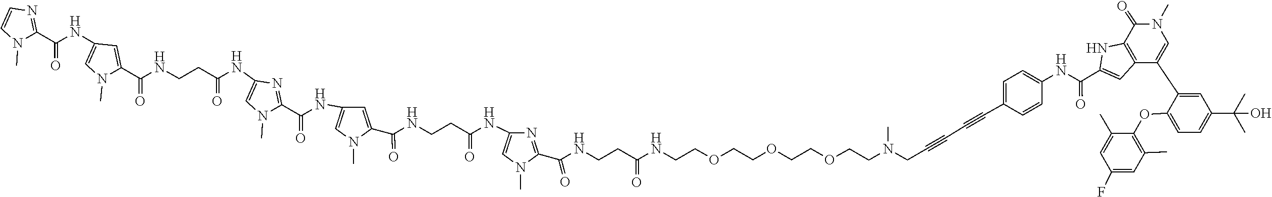 [SMILES: Cc1cc(F)cc(C)c1Oc1ccc(C(C)(C)O)cc1-c1cn(C)c(=O)c2[nH]c(C(=O)Nc3ccc(C#CC#CCN(C)CCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)cc3)cc12] | 183 |
| 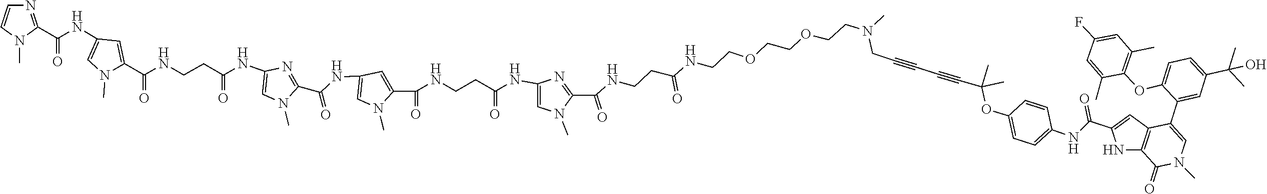 [SMILES: Cc1cc(F)cc(C)c1Oc1ccc(C(C)(C)O)cc1-c1cn(C)c(=O)c2[nH]c(C(=O)Nc3ccc(OC(C)(C)C#CC#CCN(C)CCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)cc3)cc12] | 184 |
| 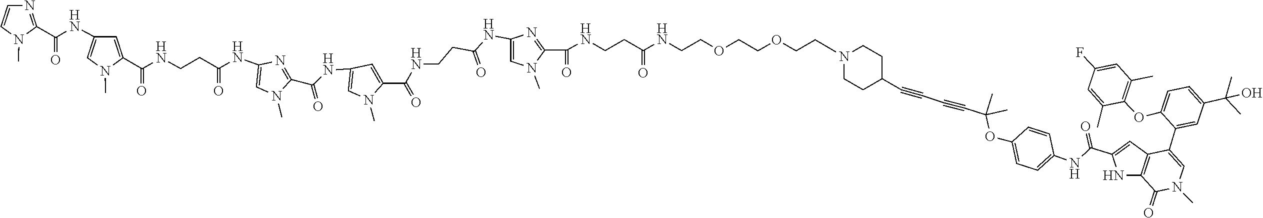 [SMILES: Cc1cc(F)cc(C)c1Oc1ccc(C(C)(C)O)cc1-c1cn(C)c(=O)c2[nH]c(C(=O)Nc3ccc(OC(C)(C)C#CC#CC4CCN(CCOCCOCCNC(=O)CCNC(=O)c5nc(NC(=O)CCNC(=O)c6cc(NC(=O)c7nc(NC(=O)CCNC(=O)c8cc(NC(=O)c9nccn9C)cn8C)cn7C)cn6C)cn5C)CC4)cc3)cc12] | 185 |
| 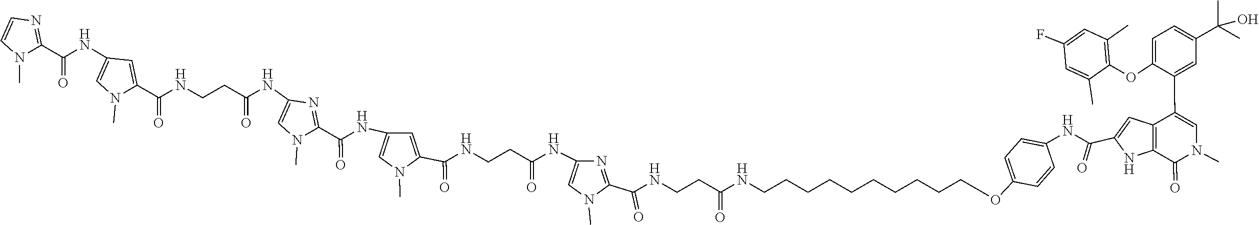 [SMILES: Cc1cc(F)cc(C)c1Oc1ccc(C(C)(C)O)cc1-c1cn(C)c(=O)c2[nH]c(C(=O)Nc3ccc(OCCCCCCCCCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)cc3)cc12] | 186 |
| 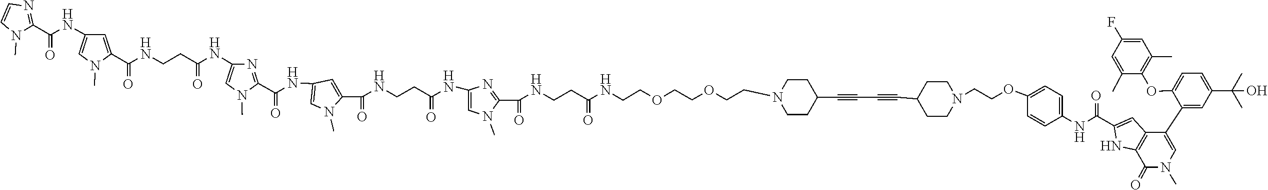 [SMILES: Cc1cc(F)cc(C)c1Oc1ccc(C(C)(C)O)cc1-c1cn(C)c(=O)c2[nH]c(C(=O)Nc3ccc(OCCN4CCC(C#CC#CC5CCN(CCOCCOCCNC(=O)CCNC(=O)c6nc(NC(=O)CCNC(=O)c7cc(NC(=O)c8nc(NC(=O)CCNC(=O)c9cc(NC(=O)c%10nccn%10C)cn9C)cn8C)cn7C)cn6C)CC5)CC4)cc3)cc12] | 187 |
| 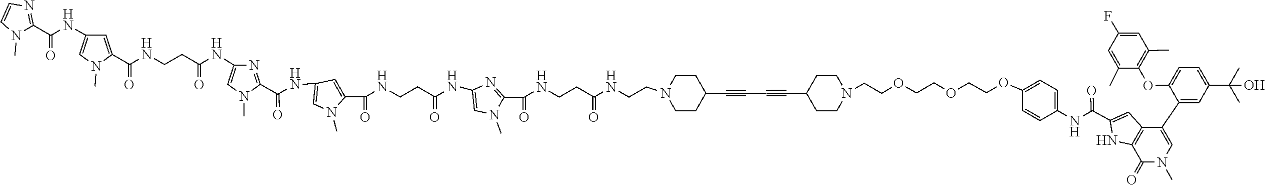 [SMILES: Cc1cc(F)cc(C)c1Oc1ccc(C(C)(C)O)cc1-c1cn(C)c(=O)c2[nH]c(C(=O)Nc3ccc(OCCOCCOCCN4CCC(C#CC#CC5CCN(CCNC(=O)CCNC(=O)c6nc(NC(=O)CCNC(=O)c7cc(NC(=O)c8nc(NC(=O)CCNC(=O)c9cc(NC(=O)c%10nccn%10C)cn9C)cn8C)cn7C)cn6C)CC5)CC4)cc3)cc12] | 188 |
| 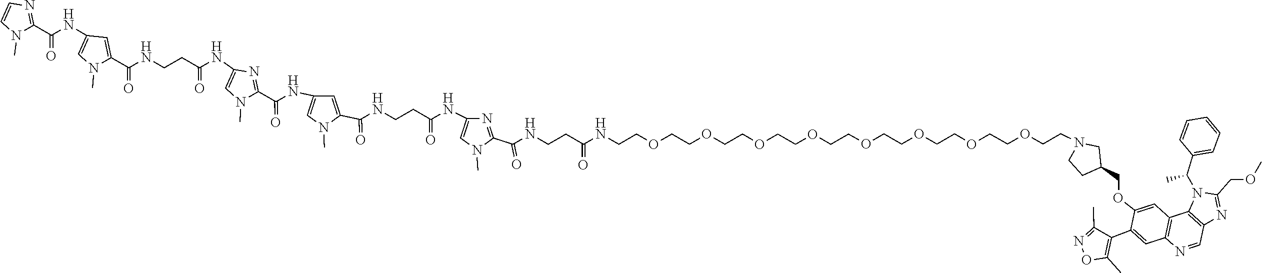 [SMILES: COCc1nc2cnc3cc(-c4c(C)noc4C)c(OC[C@H]4CCN(CCOCCOCCOCCOCCOCCOCCOCCOCCNC(=O)CCNC(=O)c5nc(NC(=O)CCNC(=O)c6cc(NC(=O)c7nc(NC(=O)CCNC(=O)c8cc(NC(=O)c9nccn9C)cn8C)cn7C)cn6C)cn5C)C4)cc3c2n1[C@H](C)c1ccccc1] | 189 |
| 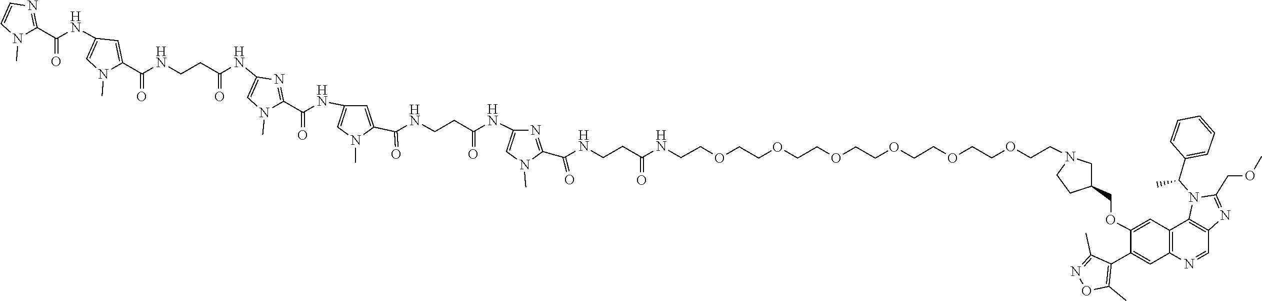 [SMILES: COCc1nc2cnc3cc(-c4c(C)noc4C)c(OC[C@H]4CCN(CCOCCOCCOCCOCCOCCOCCNC(=O)CCNC(=O)c5nc(NC(=O)CCNC(=O)c6cc(NC(=O)c7nc(NC(=O)CCNC(=O)c8cc(NC(=O)c9nccn9C)cn8C)cn7C)cn6C)cn5C)C4)cc3c2n1[C@H](C)c1ccccc1] | 190 |
| 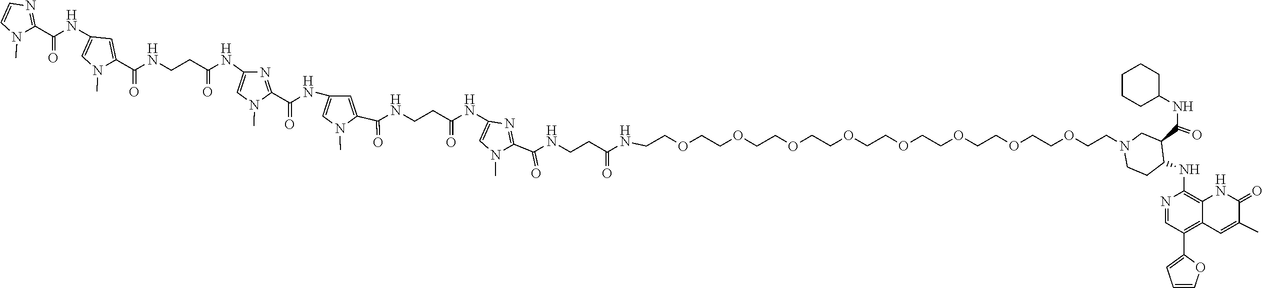 | 191 |
| 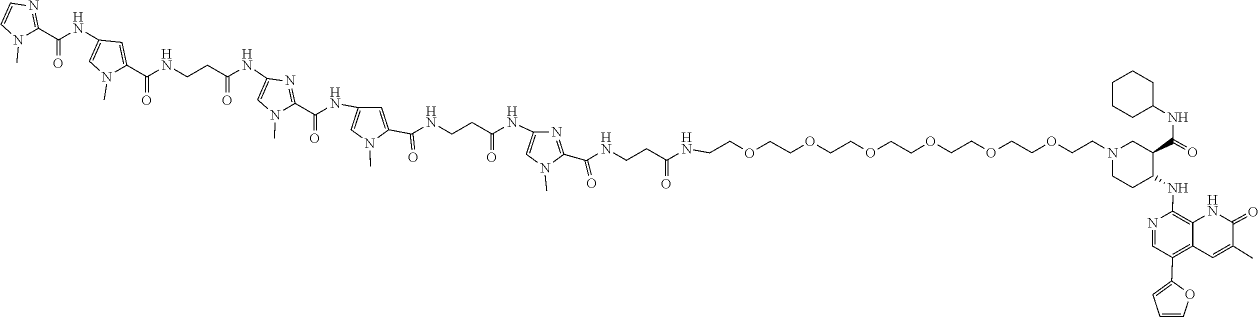 | 192 |
| 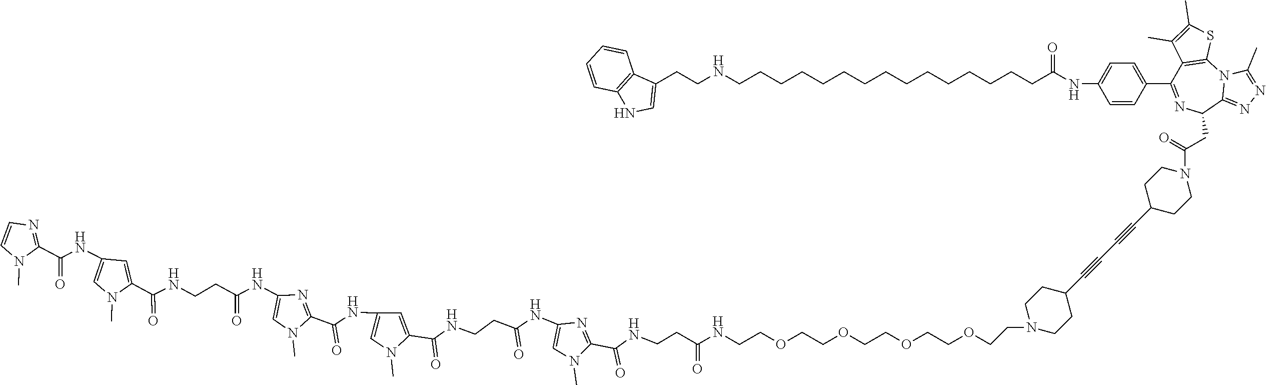 [SMILES: Cc1sc2c(c1C)C(c1ccc(NC(=O)CCCCCCCCCCCCCCCNCCc3c[nH]c4ccccc34)cc1)=N[C@@H](CC(=O)N1CCC(C#CC#CC3CCN(CCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)CC3)CC1)c1nnc(C)n1-2] | 193 |
| 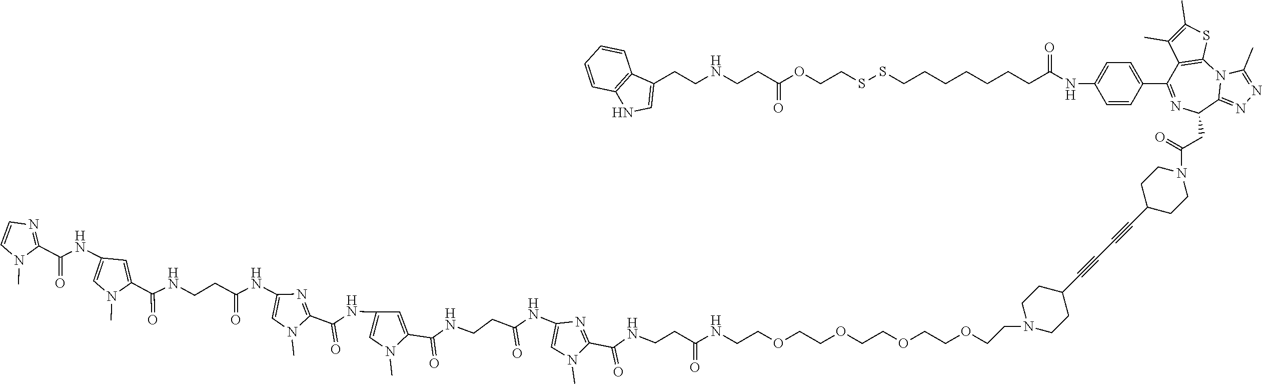 [SMILES: Cc1sc2c(c1C)C(c1ccc(NC(=O)CCCCCCCSSCCOC(=O)CCNCCc3c[nH]c4ccccc34)cc1)=N[C@@H](CC(=O)N1CCC(C#CC#CC3CCN(CCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)CC3)CC1)c1nnc(C)n1-2] | 194 |
| 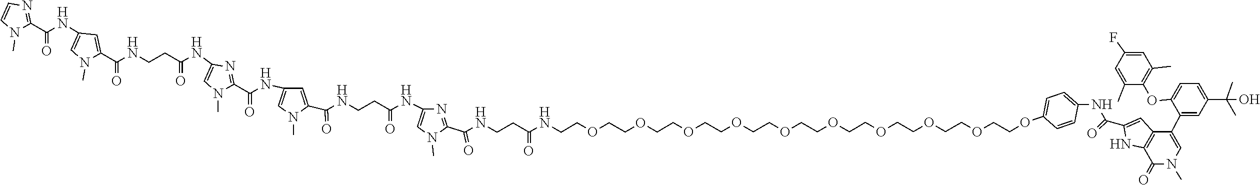 [SMILES: Cc1cc(F)cc(C)c1Oc1ccc(C(C)(C)O)cc1-c1cn(C)c(=O)c2[nH]c(C(=O)Nc3ccc(OCCOCCOCCOCCOCCOCCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)cc3)cc12] | 195 |
| 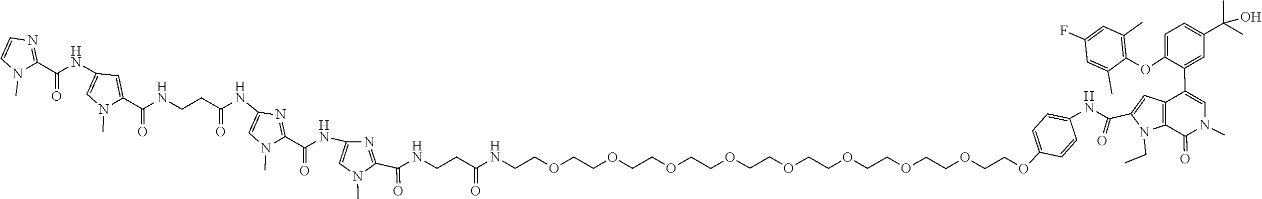 [SMILES: CCn1c(C(=O)Nc2ccc(OCCOCCOCCOCCOCCOCCOCCOCCOCCNC(=O)CCNC(=O)c3nc(NC(=O)c4nc(NC(=O)CCNC(=O)c5cc(NC(=O)c6nccn6C)cn5C)cn4C)cn3C)cc2)cc2c(-c3cc(C(C)(C)O)ccc3Oc3c(C)cc(F)cc3C)cn(C)c(=O)c21] | 196 |
| 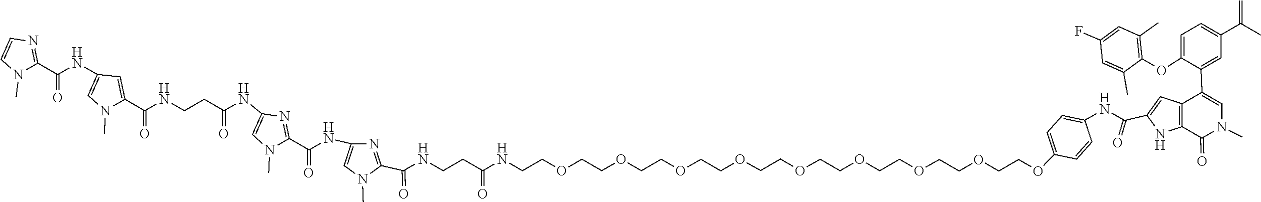 [SMILES: C=C(C)c1ccc(Oc2c(C)cc(F)cc2C)c(-c2cn(C)c(=O)c3[nH]c(C(=O)Nc4ccc(OCCOCCOCCOCCOCCOCCOCCOCCOCCNC(=O)CCNC(=O)c5nc(NC(=O)c6nc(NC(=O)CCNC(=O)c7cc(NC(=O)c8nccn8C)cn7C)cn6C)cn5C)cc4)cc23)c1] | 197 |
| 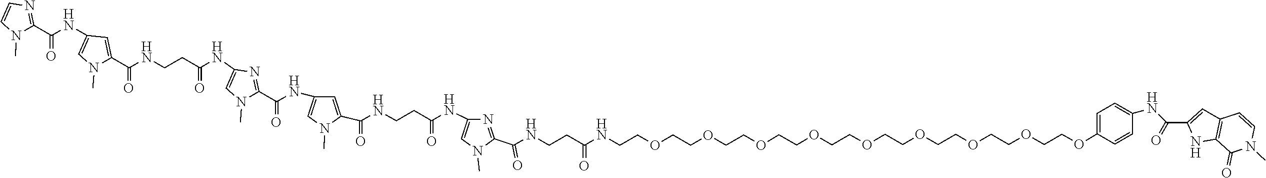 [SMILES: Cn1cc(NC(=O)c2nc(NC(=O)CCNC(=O)c3cc(NC(=O)c4nccn4C)cn3C)cn2C)cc1C(=O)NCCC(=O)Nc1cn(C)c(C(=O)NCCC(=O)NCCOCCOCCOCCOCCOCCOCCOCCOCCOc2ccc(NC(=O)c3cc4ccn(C)c(=O)c4[nH]3)cc2)n1] | 198 |
| 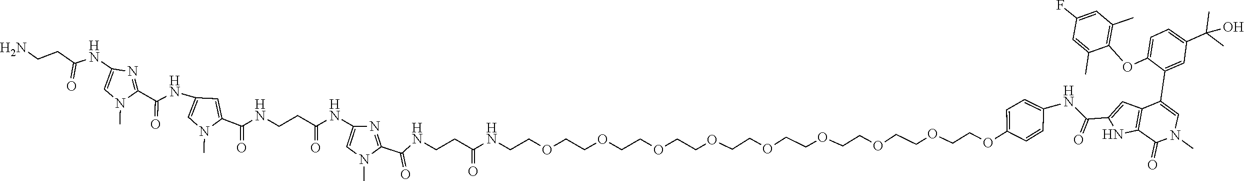 [SMILES: Cc1cc(F)cc(C)c1Oc1ccc(C(C)(C)O)cc1-c1cn(C)c(=O)c2[nH]c(C(=O)Nc3ccc(OCCOCCOCCOCCOCCOCCOCCOCCOCCNC(=O)CCNC(=O)c4nc(NC(=O)CCNC(=O)c5cc(NC(=O)c6nc(NC(=O)CCN)cn6C)cn5C)cn4C)cc3)cc12] | 199 |
| 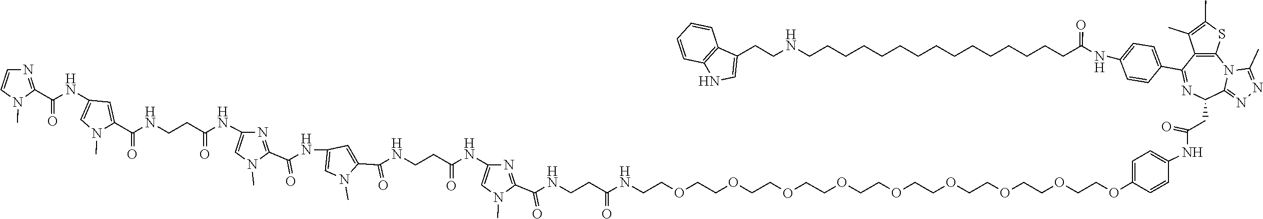 | 200 |
| 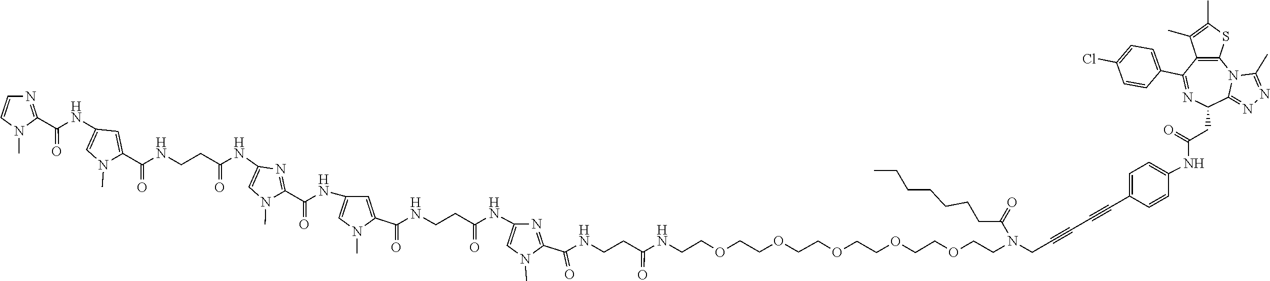 [SMILES: CCCCCCCC(=O)N(CC#CC#Cc1ccc(NC(=O)C[C@@H]2N=C(c3ccc(Cl)cc3)c3c(sc(C)c3C)-n3c(C)nnc32)cc1)CCOCCOCCOCCOCCOCCNC(=O)CCNC(=O)c1nc(NC(=O)CCNC(=O)c2cc(NC(=O)c3nc(NC(=O)CCNC(=O)c4cc(NC(=O)c5nccn5C)cn4C)cn3C)cn2C)cn1C] | 201 |
| 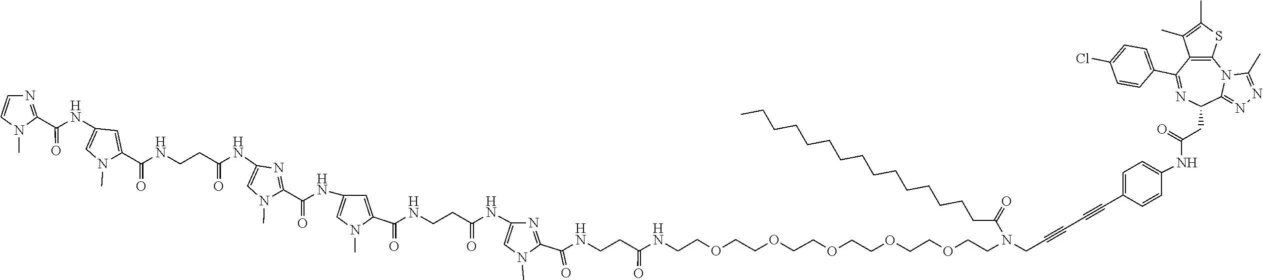 [SMILES: CCCCCCCCCCCCCCCC(=O)N(CC#CC#Cc1ccc(NC(=O)C[C@@H]2N=C(c3ccc(Cl)cc3)c3c(sc(C)c3C)-n3c(C)nnc32)cc1)CCOCCOCCOCCOCCOCCNC(=O)CCNC(=O)c1nc(NC(=O)CCNC(=O)c2cc(NC(=O)c3nc(NC(=O)CCNC(=O)c4cc(NC(=O)c5nccn5C)cn4C)cn3C)cn2C)cn1C] | 202 |
| 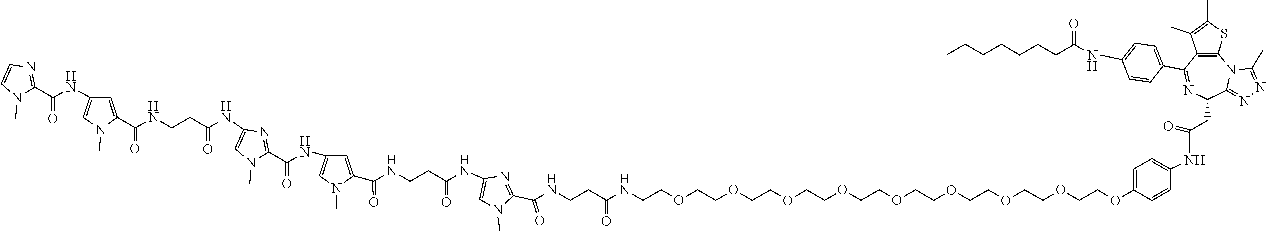 [SMILES: CCCCCCCC(=O)Nc1ccc(C2=N[C@@H](CC(=O)Nc3ccc(OCCOCCOCCOCCOCCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)cc3)c3nnc(C)n3-c3sc(C)c(C)c32)cc1] | 203 |
| 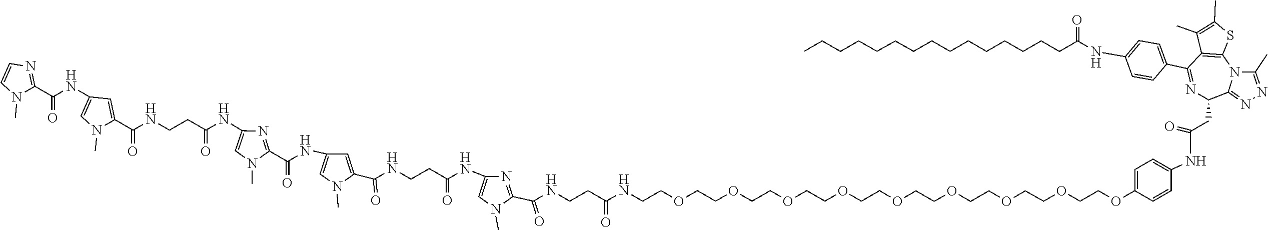 [SMILES: CCCCCCCCCCCCCCCC(=O)Nc1ccc(C2=N[C@@H](CC(=O)Nc3ccc(OCCOCCOCCOCCOCCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)cc3)c3nnc(C)n3-c3sc(C)c(C)c32)cc1] | 204 |
| 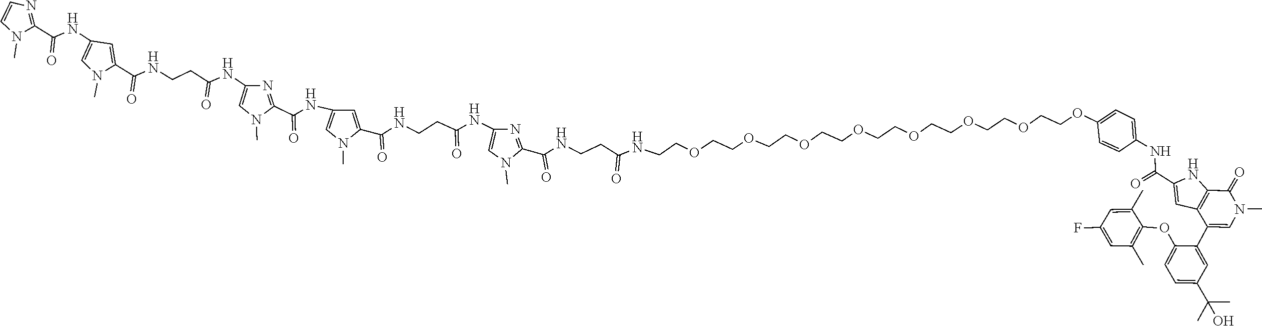 [SMILES: Cc1cc(F)cc(C)c1Oc1ccc(C(C)(C)O)cc1-c1cn(C)c(=O)c2[nH]c(C(=O)Nc3ccc(OCCOCCOCCOCCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)cc3)cc12] | 205 |
| 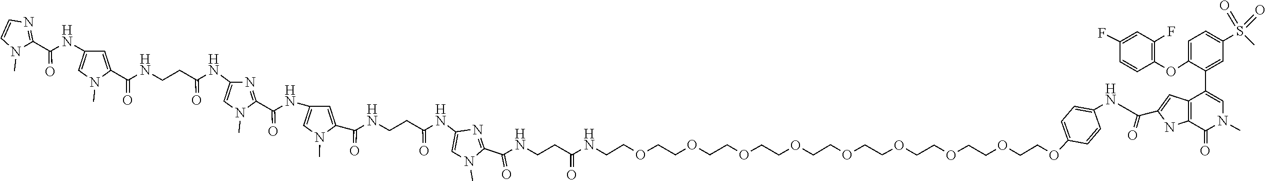 | 207 |
| 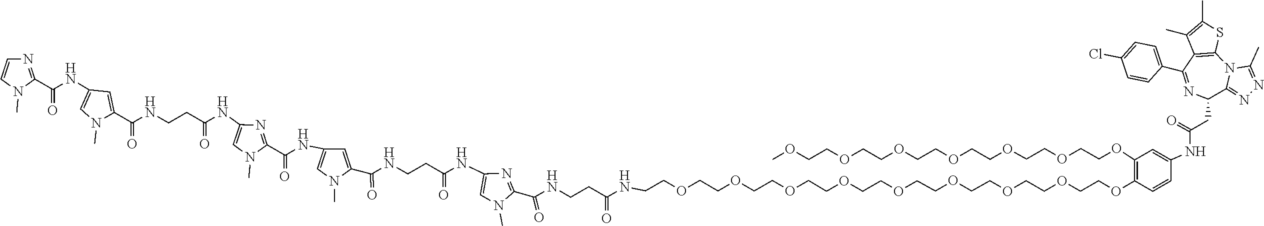 | 211 |
| 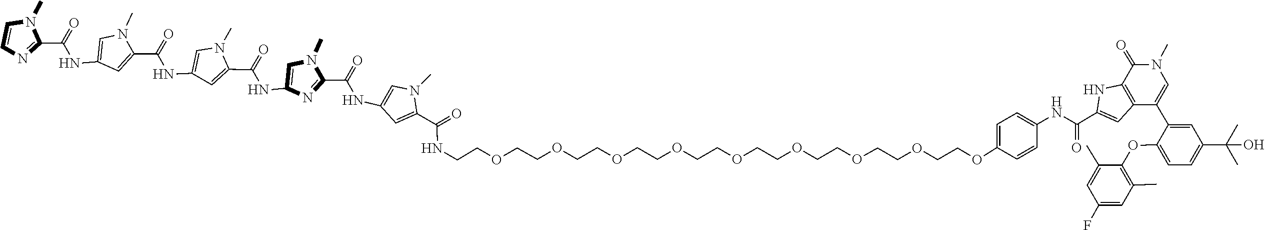 | 214 |
| 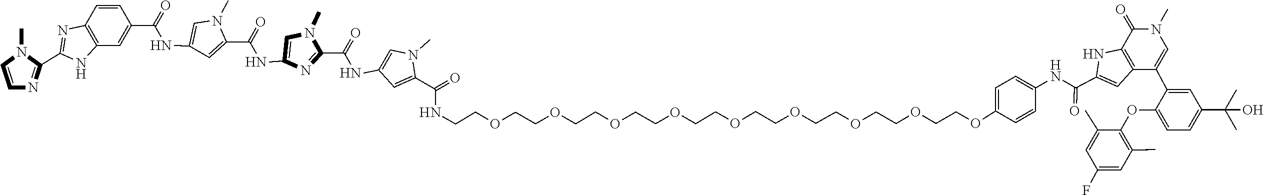 | 215 |
| 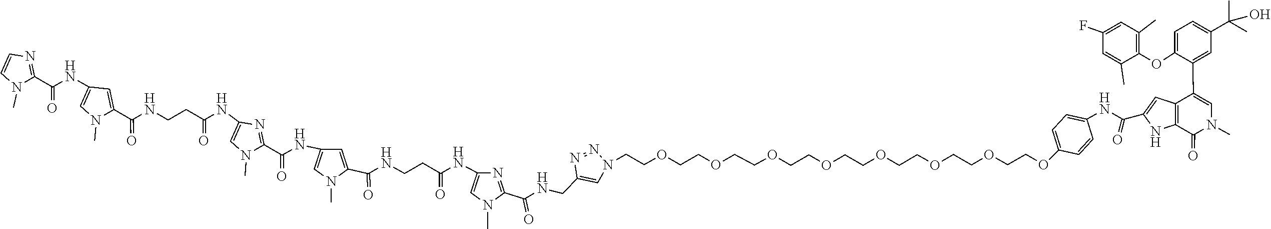 [SMILES: Cc1cc(F)cc(C)c1Oc1ccc(C(C)(C)O)cc1-c1cn(C)c(=O)c2[nH]c(C(=O)Nc3ccc(OCCOCCOCCOCCOCCOCCOCCOCCn4cc(CNC(=O)c5nc(NC(=O)CCNC(=O)c6cc(NC(=O)c7nc(NC(=O)CCNC(=O)c8cc(NC(=O)c9nccn9C)cn8C)cn7C)cn6C)cn5C)nn4)cc3)cc12] | 217 |
| 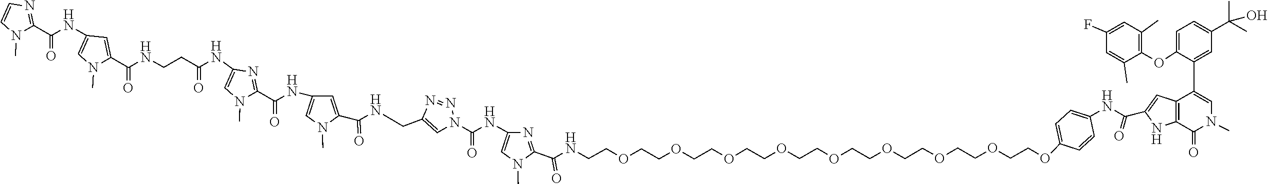 [SMILES: Cc1cc(F)cc(C)c1Oc1ccc(C(C)(C)O)cc1-c1cn(C)c(=O)c2[nH]c(C(=O)Nc3ccc(OCCOCCOCCOCCOCCOCCOCCOCCOCCNC(=O)c4nc(NC(=O)n5cc(CNC(=O)c6cc(NC(=O)c7nc(NC(=O)CCNC(=O)c8cc(NC(=O)c9nccn9C)cn8C)cn7C)cn6C)nn5)cn4C)cc3)cc12] | 218 |
| 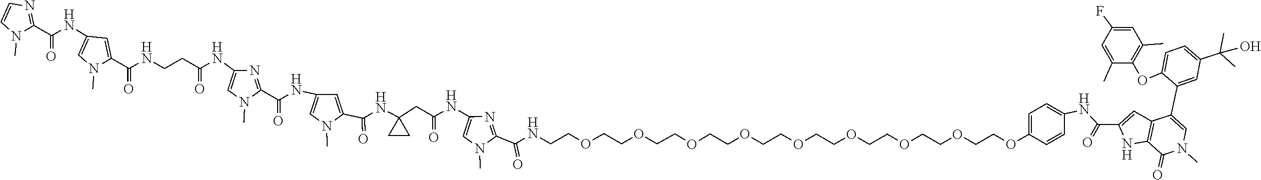 [SMILES: Cc1cc(F)cc(C)c1Oc1ccc(C(C)(C)O)cc1-c1cn(C)c(=O)c2[nH]c(C(=O)Nc3ccc(OCCOCCOCCOCCOCCOCCOCCOCCOCCNC(=O)c4nc(NC(=O)CC5(NC(=O)c6cc(NC(=O)c7nc(NC(=O)CCNC(=O)c8cc(NC(=O)c9nccn9C)cn8C)cn7C)cn6C)CC5)cn4C)cc3)cc12] | 219 |
| 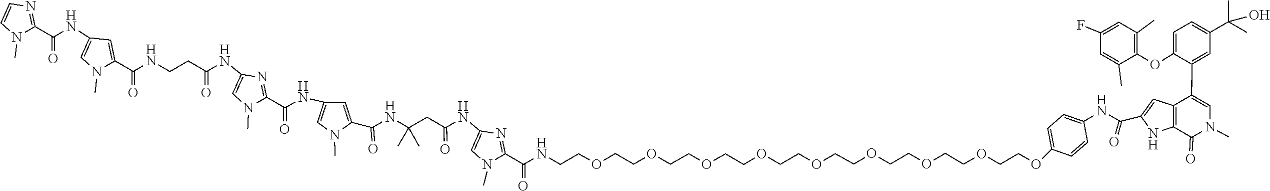 [SMILES: Cc1cc(F)cc(C)c1Oc1ccc(C(C)(C)O)cc1-c1cn(C)c(=O)c2[nH]c(C(=O)Nc3ccc(OCCOCCOCCOCCOCCOCCOCCOCCOCCNC(=O)c4nc(NC(=O)CC(C)(C)NC(=O)c5cc(NC(=O)c6nc(NC(=O)CCNC(=O)c7cc(NC(=O)c8nccn8C)cn7C)cn6C)cn5C)cn4C)cc3)cc12] | 220 |
| 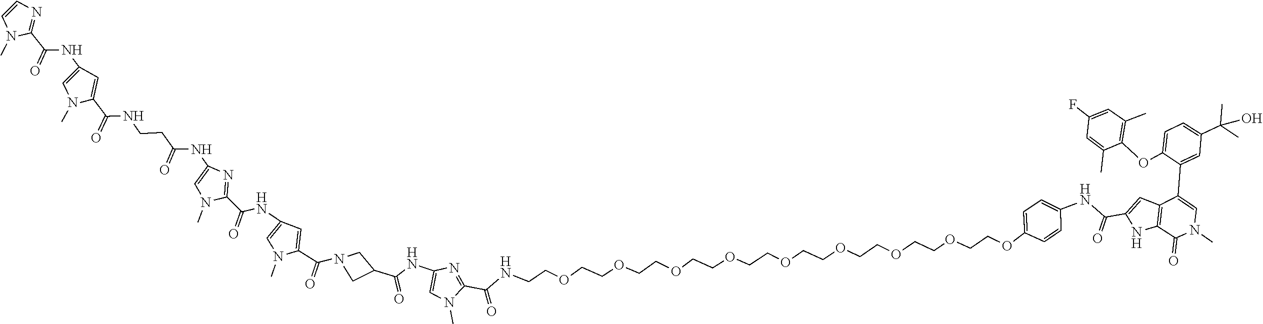 [SMILES: Cc1cc(F)cc(C)c1Oc1ccc(C(C)(C)O)cc1-c1cn(C)c(=O)c2[nH]c(C(=O)Nc3ccc(OCCOCCOCCOCCOCCOCCOCCOCCOCCNC(=O)c4nc(NC(=O)C5CN(C(=O)c6cc(NC(=O)c7nc(NC(=O)CCNC(=O)c8cc(NC(=O)c9nccn9C)cn8C)cn7C)cn6C)C5)cn4C)cc3)cc12] | 222 |
| 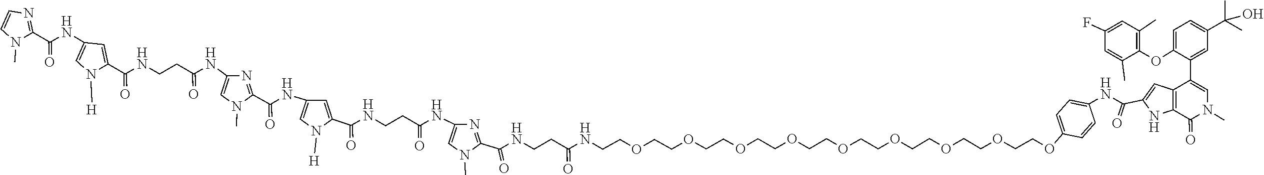 [SMILES: Cc1cc(F)cc(C)c1Oc1ccc(C(C)(C)O)cc1-c1cn(C)c(=O)c2[nH]c(C(=O)Nc3ccc(OCCOCCOCCOCCOCCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)c[nH]7)cn6C)c[nH]5)cn4C)cc3)cc12] | 224 |
| 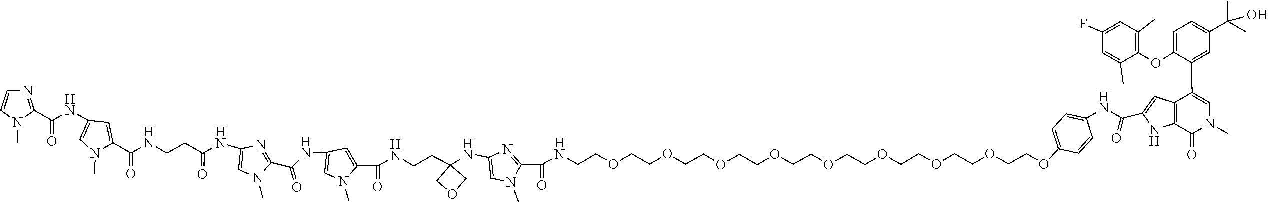 [SMILES: Cc1cc(F)cc(C)c1Oc1ccc(C(C)(C)O)cc1-c1cn(C)c(=O)c2[nH]c(C(=O)Nc3ccc(OCCOCCOCCOCCOCCOCCOCCOCCOCCNC(=O)c4nc(NC5(CCNC(=O)c6cc(NC(=O)c7nc(NC(=O)CCNC(=O)c8cc(NC(=O)c9nccn9C)cn8C)cn7C)cn6C)COC5)cn4C)cc3)cc12] | 225 |
| 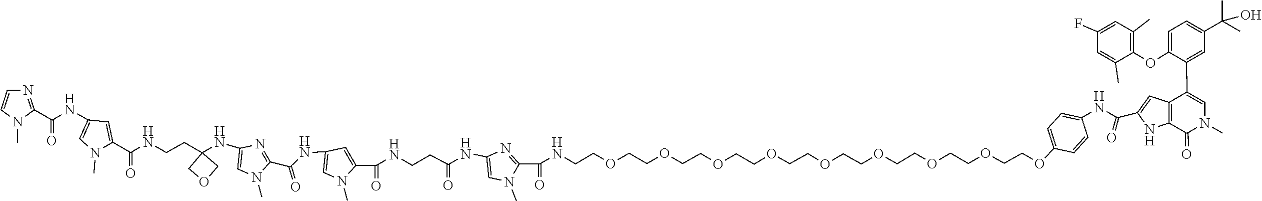 [SMILES: Cc1cc(F)cc(C)c1Oc1ccc(C(C)(C)O)cc1-c1cn(C)c(=O)c2[nH]c(C(=O)Nc3ccc(OCCOCCOCCOCCOCCOCCOCCOCCOCCNC(=O)c4nc(NC(=O)CCNC(=O)c5cc(NC(=O)c6nc(NC7(CCNC(=O)c8cc(NC(=O)c9nccn9C)cn8C)COC7)cn6C)cn5C)cn4C)cc3)cc12] | 226 |
| 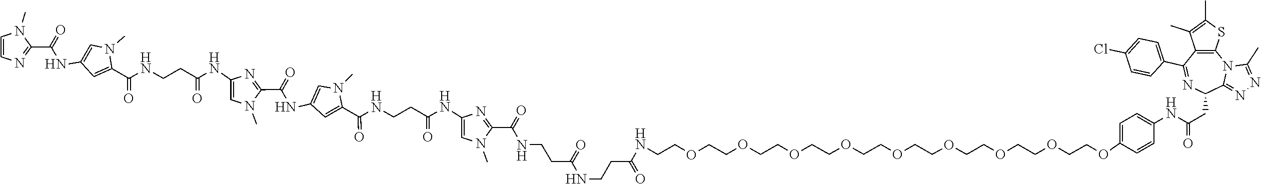 [SMILES: Cc1sc2c(c1C)C(c1ccc(Cl)cc1)=N[C@@H](CC(=O)Nc1ccc(OCCOCCOCCOCCOCCOCCOCCOCCOCCNC(=O)CCNC(=O)CCNC(=O)c3nc(NC(=O)CCNC(=O)c4cc(NC(=O)c5nc(NC(=O)CCNC(=O)c6cc(NC(=O)c7nccn7C)cn6C)cn5C)cn4C)cn3C)cc1)c1nnc(C)n1-2] | 229 |
| 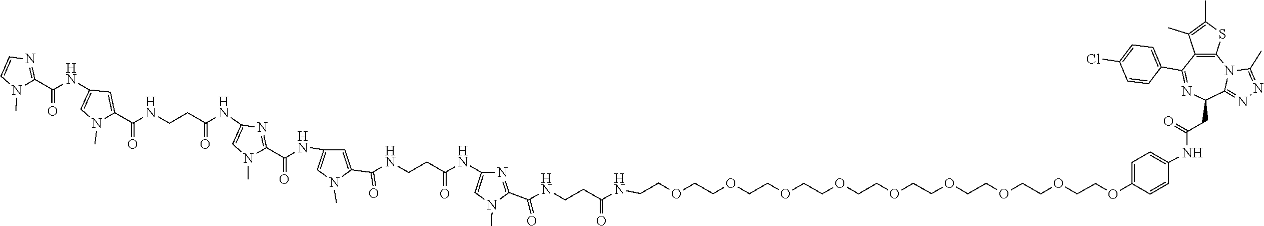 [SMILES: Cc1sc2c(c1C)C(c1ccc(Cl)cc1)=N[C@H](CC(=O)Nc1ccc(OCCOCCOCCOCCOCCOCCOCCOCCOCCNC(=O)CCNC(=O)c3nc(NC(=O)CCNC(=O)c4cc(NC(=O)c5nc(NC(=O)CCNC(=O)c6cc(NC(=O)c7nccn7C)cn6C)cn5C)cn4C)cn3C)cc1)c1nnc(C)n1-2] | 231 |
| 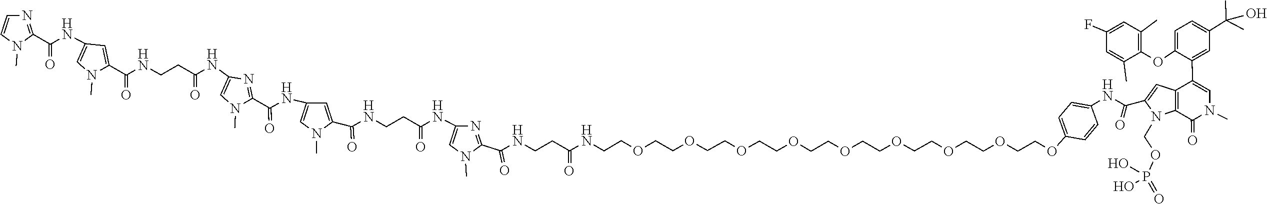 [SMILES: Cc1cc(F)cc(C)c1Oc1ccc(C(C)(C)O)cc1-c1cn(C)c(=O)c2c1cc(C(=O)Nc1ccc(OCCOCCOCCOCCOCCOCCOCCOCCOCCNC(=O)CCNC(=O)c3nc(NC(=O)CCNC(=O)c4cc(NC(=O)c5nc(NC(=O)CCNC(=O)c6cc(NC(=O)c7nccn7C)cn6C)cn5C)cn4C)cn3C)cc1)n2COP(=O)(O)O] | 233 |
| 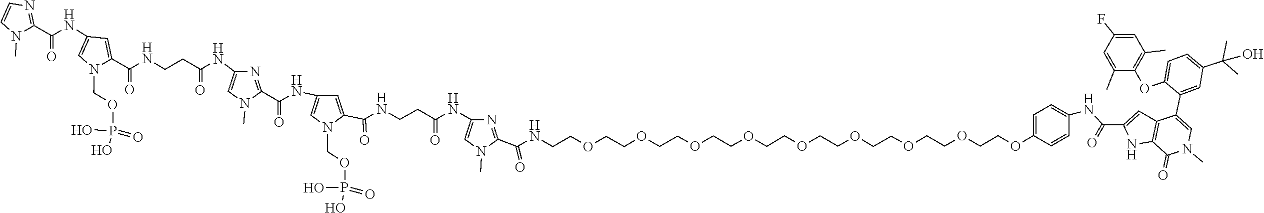 [SMILES: Cc1cc(F)cc(C)c1Oc1ccc(C(C)(C)O)cc1-c1cn(C)c(=O)c2[nH]c(C(=O)Nc3ccc(OCCOCCOCCOCCOCCOCCOCCOCCOCCNC(=O)c4nc(NC(=O)CCNC(=O)c5cc(NC(=O)c6nc(NC(=O)CCNC(=O)c7cc(NC(=O)c8nccn8C)cn7COP(=O)(O)O)cn6C)cn5COP(=O)(O)O)cn4C)cc3)cc12] | 235 |
| 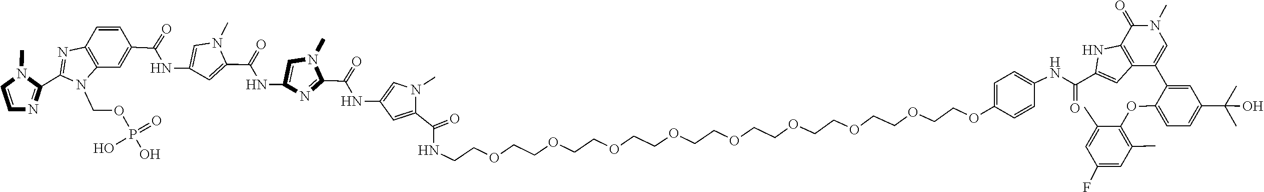 | 236 |
| 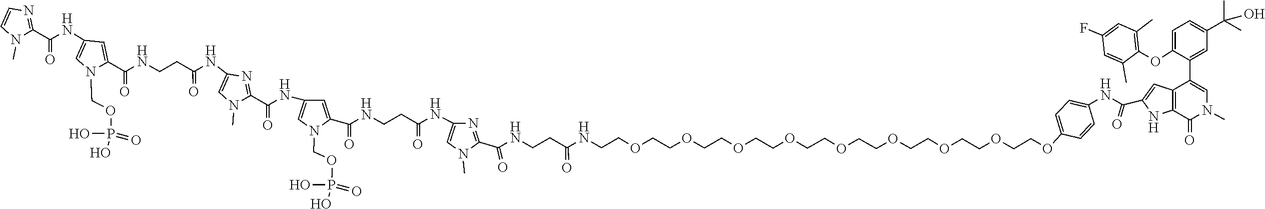 [SMILES: Cc1cc(F)cc(C)c1Oc1ccc(C(C)(C)O)cc1-c1cn(C)c(=O)c2[nH]c(C(=O)Nc3ccc(OCCOCCOCCOCCOCCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7COP(=O)(O)O)cn6C)cn5COP(=O)(O)O)cn4C)cc3)cc12] | 237 |
| 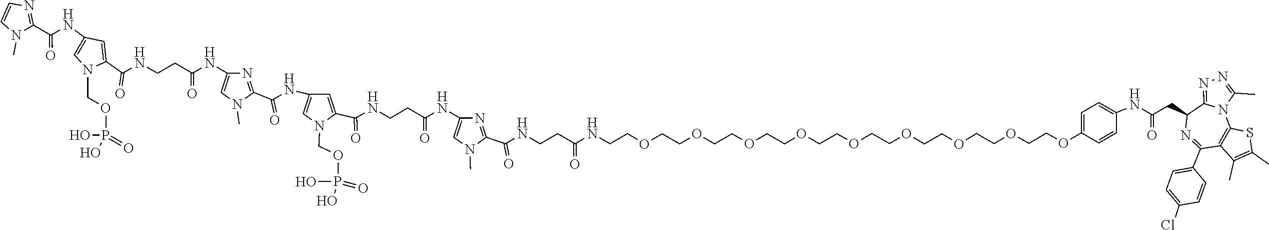 [SMILES: Cc1sc2c(c1C)C(c1ccc(Cl)cc1)=N[C@@H](CC(=O)Nc1ccc(OCCOCCOCCOCCOCCOCCOCCOCCOCCNC(=O)CCNC(=O)c3nc(NC(=O)CCNC(=O)c4cc(NC(=O)c5nc(NC(=O)CCNC(=O)c6cc(NC(=O)c7nccn7C)cn6COP(=O)(O)O)cn5C)cn4COP(=O)(O)O)cn3C)cc1)c1nnc(C)n1-2] | 238 |
| 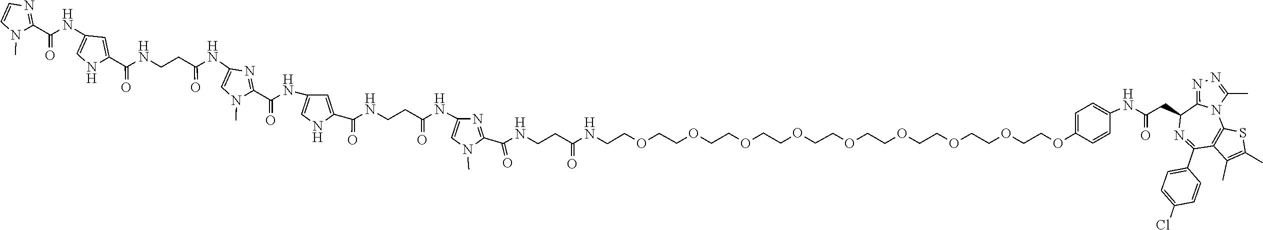 [SMILES: Cc1sc2c(c1C)C(c1ccc(Cl)cc1)=N[C@@H](CC(=O)Nc1ccc(OCCOCCOCCOCCOCCOCCOCCOCCOCCNC(=O)CCNC(=O)c3nc(NC(=O)CCNC(=O)c4cc(NC(=O)c5nc(NC(=O)CCNC(=O)c6cc(NC(=O)c7nccn7C)c[nH]6)cn5C)c[nH]4)cn3C)cc1)c1nnc(C)n1-2] | 239 |
| 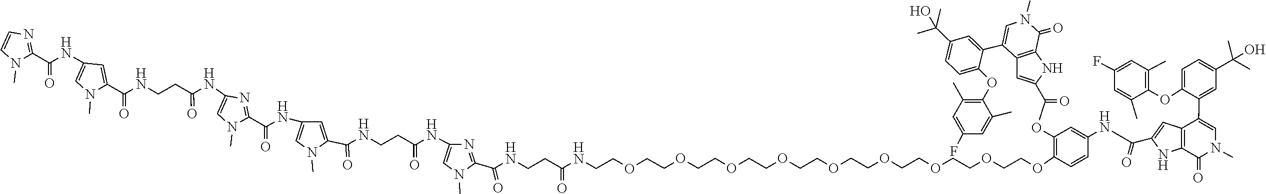 [SMILES: Cc1cc(F)cc(C)c1Oc1ccc(C(C)(C)O)cc1-c1cn(C)c(=O)c2[nH]c(C(=O)Nc3ccc(OCCOCCOCCOCCOCCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5C)cn4C)c(OC(=O)c4cc5c(-c6cc(C(C)(C)O)ccc6Oc6c(C)cc(F)cc6C)cn(C)c(=O)c5[nH]4)c3)cc12] | 240 |
| 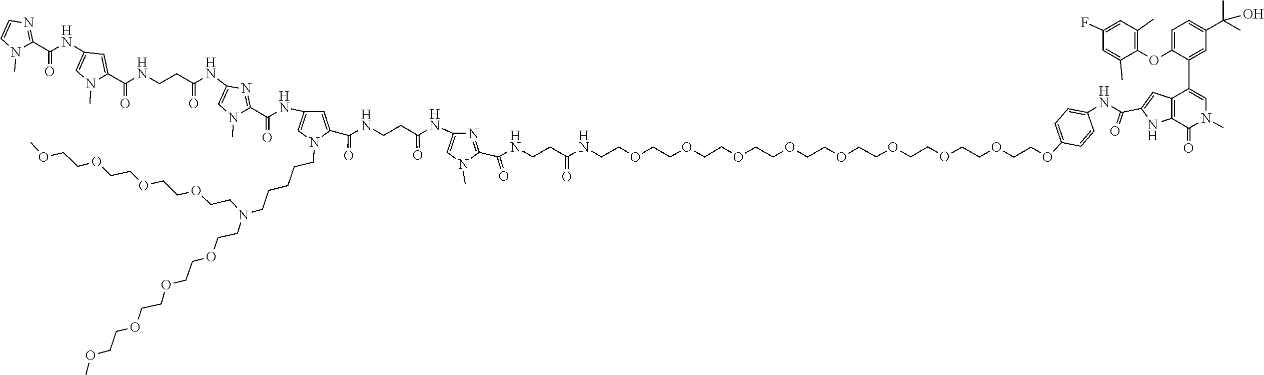 [SMILES: COCCOCCOCCOCCN(CCCCCn1cc(NC(=O)c2nc(NC(=O)CCNC(=O)c3cc(NC(=O)c4nccn4C)cn3C)cn2C)cc1C(=O)NCCC(=O)Nc1cn(C)c(C(=O)NCCC(=O)NCCOCCOCCOCCOCCOCCOCCOCCOCCOc2ccc(NC(=O)c3cc4c(-c5cc(C(C)(C)O)ccc5Oc5c(C)cc(F)cc5C)cn(C)c(=O)c4[nH]3)cc2)n1)CCOCCOCCOCCOC] | 241 |
| 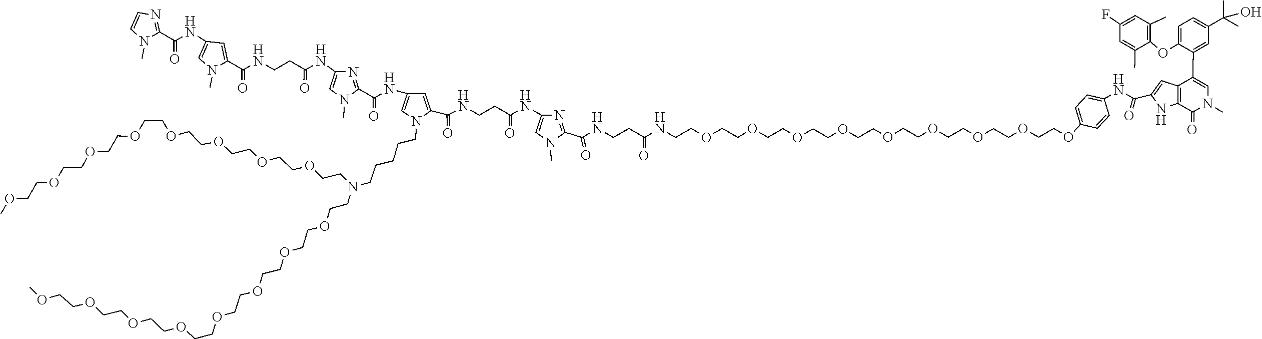 [SMILES: COCCOCCOCCOCCOCCOCCOCCOCCN(CCCCCn1cc(NC(=O)c2nc(NC(=O)CCNC(=O)c3cc(NC(=O)c4nccn4C)cn3C)cn2C)cc1C(=O)NCCC(=O)Nc1cn(C)c(C(=O)NCCC(=O)NCCOCCOCCOCCOCCOCCOCCOCCOCCOc2ccc(NC(=O)c3cc4c(-c5cc(C(C)(C)O)ccc5Oc5c(C)cc(F)cc5C)cn(C)c(=O)c4[nH]3)cc2)n1)CCOCCOCCOCCOCCOCCOCCOCCOC] | 242 |
| 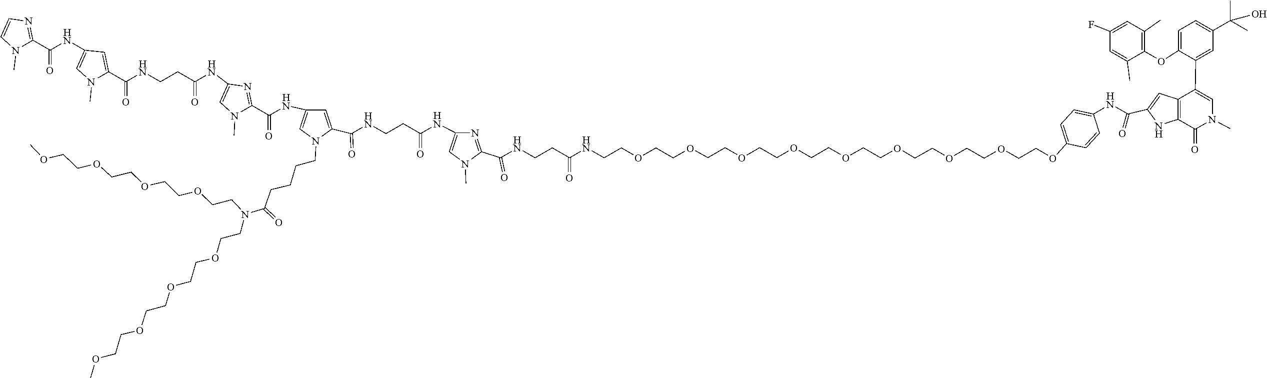 [SMILES: COCCOCCOCCOCCN(CCOCCOCCOCCOC)C(=O)CCCCn1cc(NC(=O)c2nc(NC(=O)CCNC(=O)c3cc(NC(=O)c4nccn4C)cn3C)cn2C)cc1C(=O)NCCC(=O)Nc1cn(C)c(C(=O)NCCC(=O)NCCOCCOCCOCCOCCOCCOCCOCCOCCOc2ccc(NC(=O)c3cc4c(-c5cc(C(C)(C)O)ccc5Oc5c(C)cc(F)cc5C)cn(C)c(=O)c4[nH]3)cc2)n1] | 243 |
| 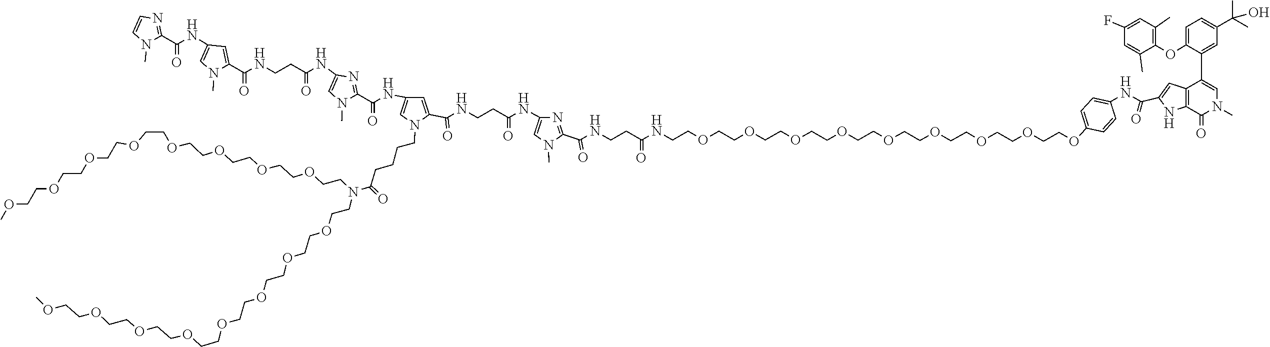 [SMILES: COCCOCCOCCOCCOCCOCCOCCOCCN(CCOCCOCCOCCOCCOCCOCCOCCOC)C(=O)CCCCn1cc(NC(=O)c2nc(NC(=O)CCNC(=O)c3cc(NC(=O)c4nccn4C)cn3C)cn2C)cc1C(=O)NCCC(=O)Nc1cn(C)c(C(=O)NCCC(=O)NCCOCCOCCOCCOCCOCCOCCOCCOCCOc2ccc(NC(=O)c3cc4c(-c5cc(C(C)(C)O)ccc5Oc5c(C)cc(F)cc5C)cn(C)c(=O)c4[nH]3)cc2)n1] | 244 |
| 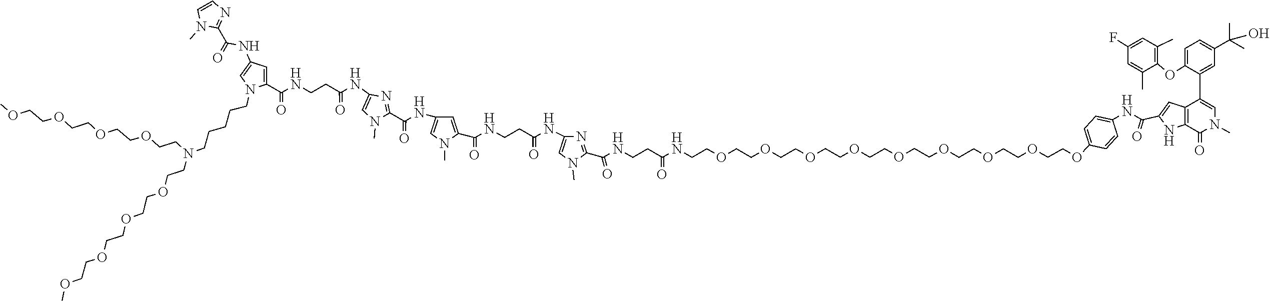 [SMILES: COCCOCCOCCOCCN(CCCCCn1cc(NC(=O)c2nccn2C)cc1C(=O)NCCC(=O)Nc1cn(C)c(C(=O)Nc2cc(C(=O)NCCC(=O)Nc3cn(C)c(C(=O)NCCC(=O)NCCOCCOCCOCCOCCOCCOCCOCCOCCOc4ccc(NC(=O)c5cc6c(-c7cc(C(C)(C)O)ccc7Oc7c(C)cc(F)cc7C)cn(C)c(=O)c6[nH]5)cc4)n3)n(C)c2)n1)CCOCCOCCOCCOC] | 245 |
| 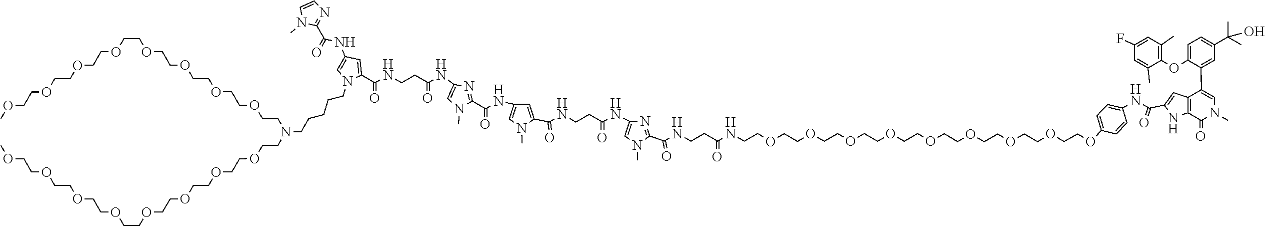 [SMILES: COCCOCCOCCOCCOCCOCCOCCOCCN(CCCCCn1cc(NC(=O)c2nccn2C)cc1C(=O)NCCC(=O)Nc1cn(C)c(C(=O)Nc2cc(C(=O)NCCC(=O)Nc3cn(C)c(C(=O)NCCC(=O)NCCOCCOCCOCCOCCOCCOCCOCCOCCOc4ccc(NC(=O)c5cc6c(-c7cc(C(C)(C)O)ccc7Oc7c(C)cc(F)cc7C)cn(C)c(=O)c6[nH]5)cc4)n3)n(C)c2)n1)CCOCCOCCOCCOCCOCCOCCOCCOC] | 246 |
| 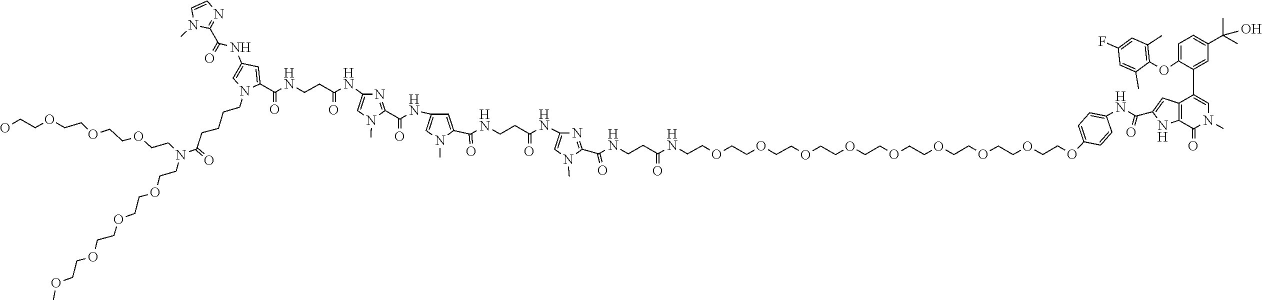 [SMILES: COCCOCCOCCOCCN(CCOCCOCCOCCO)C(=O)CCCCn1cc(NC(=O)c2nccn2C)cc1C(=O)NCCC(=O)Nc1cn(C)c(C(=O)Nc2cc(C(=O)NCCC(=O)Nc3cn(C)c(C(=O)NCCC(=O)NCCOCCOCCOCCOCCOCCOCCOCCOCCOc4ccc(NC(=O)c5cc6c(-c7cc(C(C)(C)O)ccc7Oc7c(C)cc(F)cc7C)cn(C)c(=O)c6[nH]5)cc4)n3)n(C)c2)n1] | 247 |
| 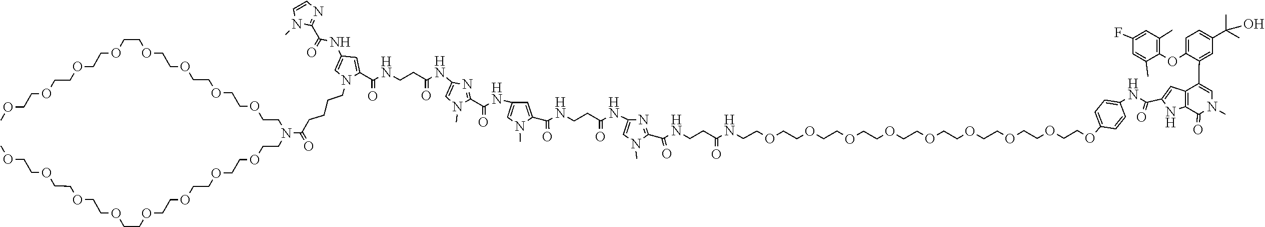 [SMILES: COCCOCCOCCOCCOCCOCCOCCOCCN(CCOCCOCCOCCOCCOCCOCCOCCOC)C(=O)CCCCn1cc(NC(=O)c2nccn2C)cc1C(=O)NCCC(=O)Nc1cn(C)c(C(=O)Nc2cc(C(=O)NCCC(=O)Nc3cn(C)c(C(=O)NCCC(=O)NCCOCCOCCOCCOCCOCCOCCOCCOCCOc4ccc(NC(=O)c5cc6c(-c7cc(C(C)(C)O)ccc7Oc7c(C)cc(F)cc7C)cn(C)c(=O)c6[nH]5)cc4)n3)n(C)c2)n1] | 248 |
| 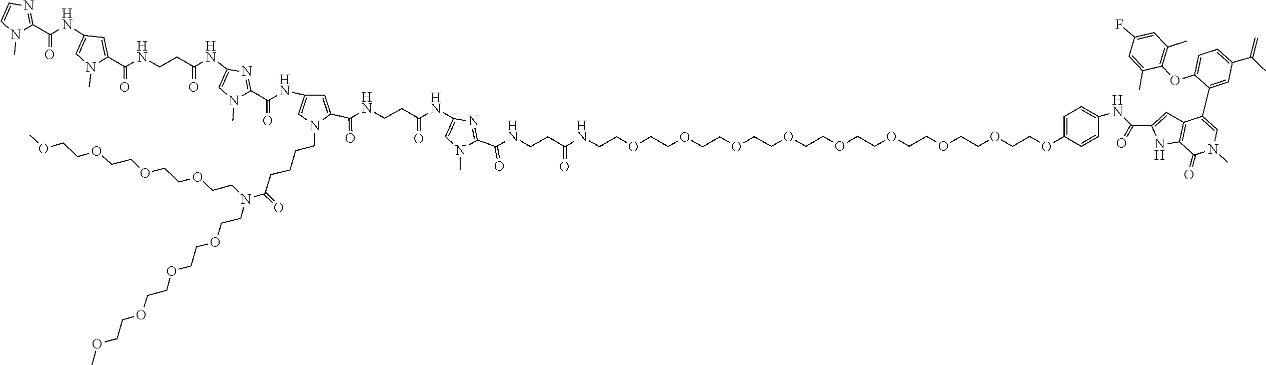 | 249 |
| 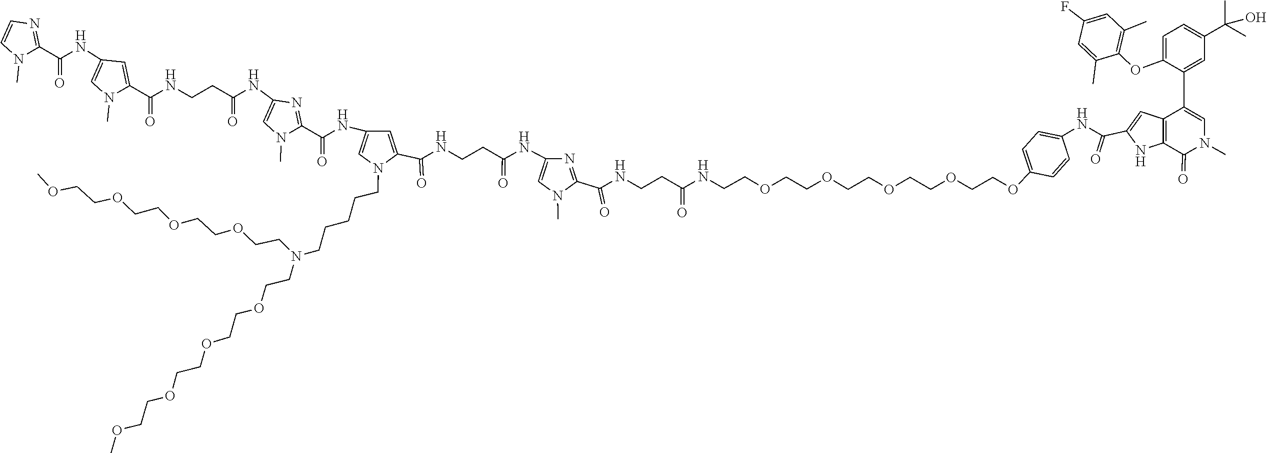 [SMILES: COCCOCCOCCOCCN(CCCCCn1cc(NC(=O)c2nc(NC(=O)CCNC(=O)c3cc(NC(=O)c4nccn4C)cn3C)cn2C)cc1C(=O)NCCC(=O)Nc1cn(C)c(C(=O)NCCC(=O)NCCOCCOCCOCCOCCOc2ccc(NC(=O)c3cc4c(-c5cc(C(C)(C)O)ccc5Oc5c(C)cc(F)cc5C)cn(C)c(=O)c4[nH]3)cc2)n1)CCOCCOCCOCCOC] | 250 |
| 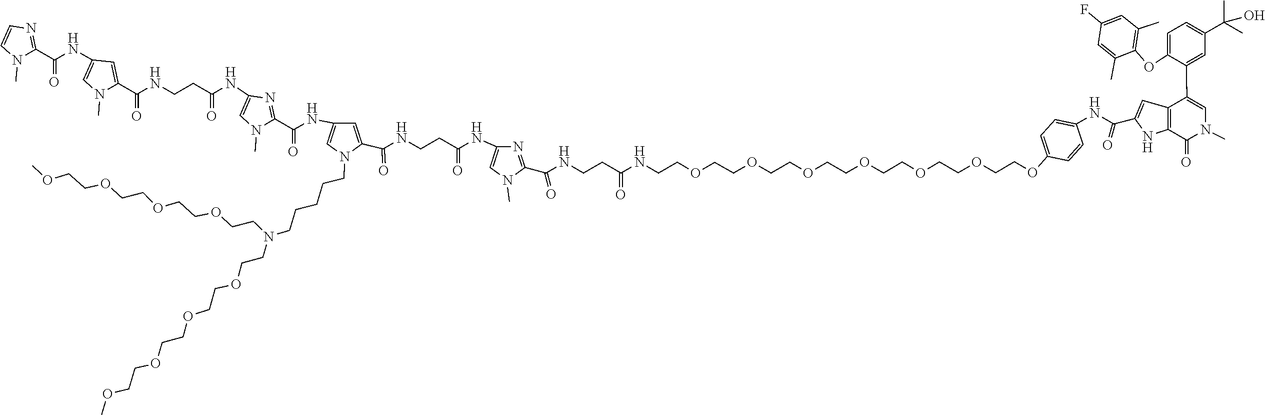 [SMILES: COCCOCCOCCOCCN(CCCCCn1cc(NC(=O)c2nc(NC(=O)CCNC(=O)c3cc(NC(=O)c4nccn4C)cn3C)cn2C)cc1C(=O)NCCC(=O)Nc1cn(C)c(C(=O)NCCC(=O)NCCOCCOCCOCCOCCOCCOCCOc2ccc(NC(=O)c3cc4c(-c5cc(C(C)(C)O)ccc5Oc5c(C)cc(F)cc5C)cn(C)c(=O)c4[nH]3)cc2)n1)CCOCCOCCOCCOC] | 251 |
| 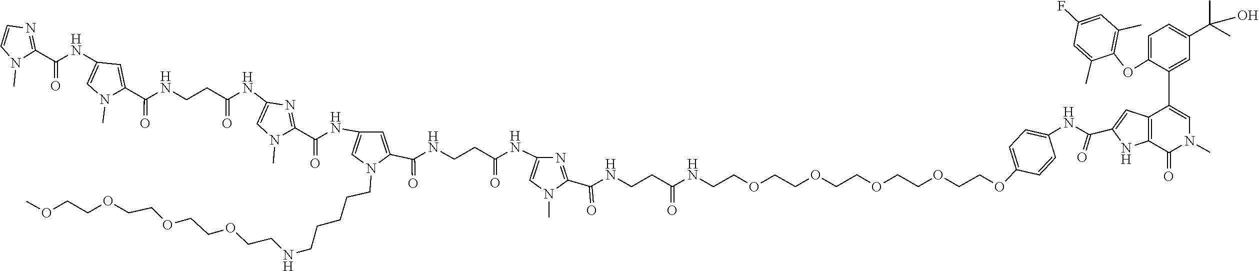 [SMILES: COCCOCCOCCOCCNCCCCCn1cc(NC(=O)c2nc(NC(=O)CCNC(=O)c3cc(NC(=O)c4nccn4C)cn3C)cn2C)cc1C(=O)NCCC(=O)Nc1cn(C)c(C(=O)NCCC(=O)NCCOCCOCCOCCOCCOc2ccc(NC(=O)c3cc4c(-c5cc(C(C)(C)O)ccc5Oc5c(C)cc(F)cc5C)cn(C)c(=O)c4[nH]3)cc2)n1] | 252 |
| 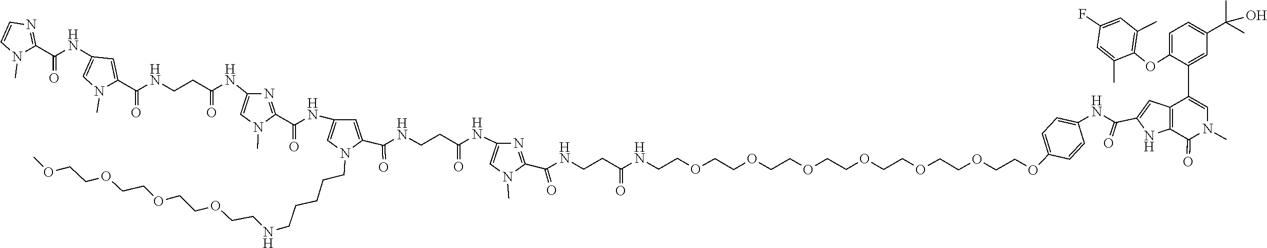 [SMILES: COCCOCCOCCOCCNCCCCCn1cc(NC(=O)c2nc(NC(=O)CCNC(=O)c3cc(NC(=O)c4nccn4C)cn3C)cn2C)cc1C(=O)NCCC(=O)Nc1cn(C)c(C(=O)NCCC(=O)NCCOCCOCCOCCOCCOCCOCCOc2ccc(NC(=O)c3cc4c(-c5cc(C(C)(C)O)ccc5Oc5c(C)cc(F)cc5C)cn(C)c(=O)c4[nH]3)cc2)n1] | 253 |
| 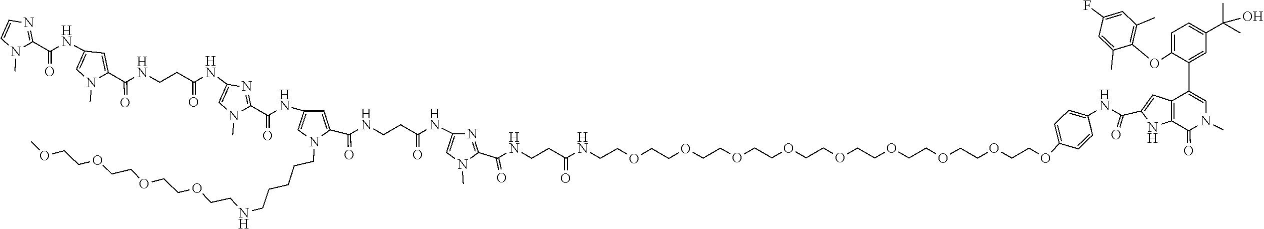 [SMILES: COCCOCCOCCOCCNCCCCCn1cc(NC(=O)c2nc(NC(=O)CCNC(=O)c3cc(NC(=O)c4nccn4C)cn3C)cn2C)cc1C(=O)NCCC(=O)Nc1cn(C)c(C(=O)NCCC(=O)NCCOCCOCCOCCOCCOCCOCCOCCOCCOc2ccc(NC(=O)c3cc4c(-c5cc(C(C)(C)O)ccc5Oc5c(C)cc(F)cc5C)cn(C)c(=O)c4[nH]3)cc2)n1] | 254 |
| 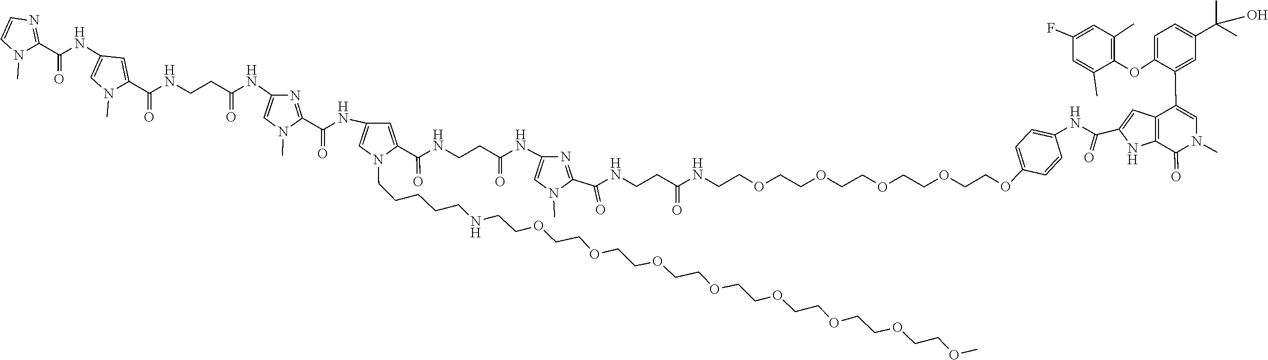 [SMILES: COCCOCCOCCOCCOCCOCCOCCOCCNCCCCCn1cc(NC(=O)c2nc(NC(=O)CCNC(=O)c3cc(NC(=O)c4nccn4C)cn3C)cn2C)cc1C(=O)NCCC(=O)Nc1cn(C)c(C(=O)NCCC(=O)NCCOCCOCCOCCOCCOc2ccc(NC(=O)c3cc4c(-c5cc(C(C)(C)O)ccc5Oc5c(C)cc(F)cc5C)cn(C)c(=O)c4[nH]3)cc2)n1] | 255 |
| 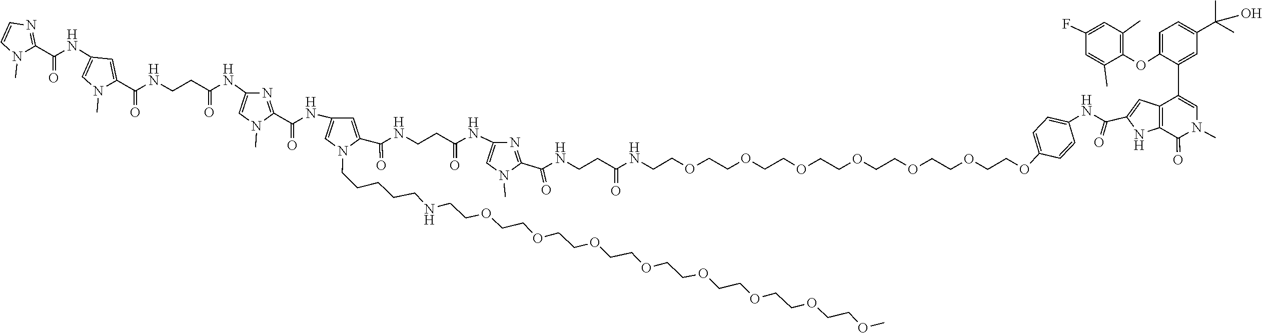 [SMILES: COCCOCCOCCOCCOCCOCCOCCOCCNCCCCCn1cc(NC(=O)c2nc(NC(=O)CCNC(=O)c3cc(NC(=O)c4nccn4C)cn3C)cn2C)cc1C(=O)NCCC(=O)Nc1cn(C)c(C(=O)NCCC(=O)NCCOCCOCCOCCOCCOCCOCCOc2ccc(NC(=O)c3cc4c(-c5cc(C(C)(C)O)ccc5Oc5c(C)cc(F)cc5C)cn(C)c(=O)c4[nH]3)cc2)n1] | 256 |
| 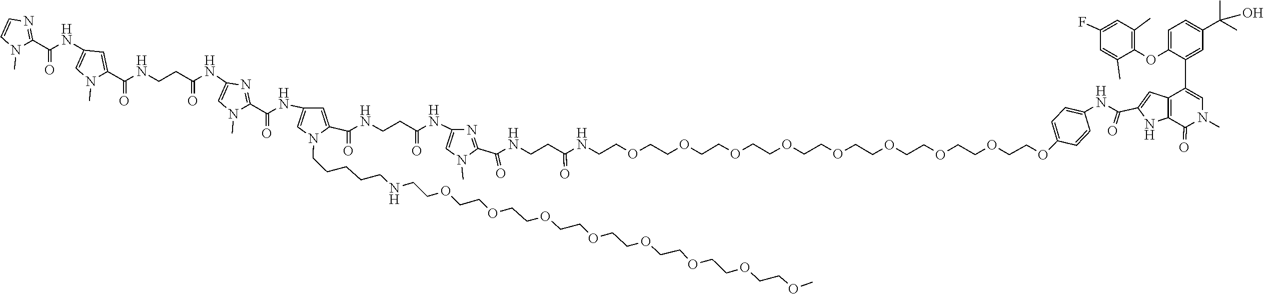 [SMILES: COCCOCCOCCOCCOCCOCCOCCOCCNCCCCCn1cc(NC(=O)c2nc(NC(=O)CCNC(=O)c3cc(NC(=O)c4nccn4C)cn3C)cn2C)cc1C(=O)NCCC(=O)Nc1cn(C)c(C(=O)NCCC(=O)NCCOCCOCCOCCOCCOCCOCCOCCOCCOc2ccc(NC(=O)c3cc4c(-c5cc(C(C)(C)O)ccc5Oc5c(C)cc(F)cc5C)cn(C)c(=O)c4[nH]3)cc2)n1] | 257 |
| 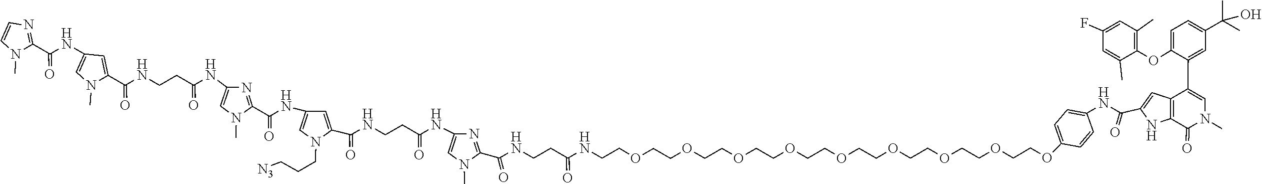 [SMILES: Cc1cc(F)cc(C)c1Oc1ccc(C(C)(C)O)cc1-c1cn(C)c(=O)c2[nH]c(C(=O)Nc3ccc(OCCOCCOCCOCCOCCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5CCCN=[N+]=[N-])cn4C)cc3)cc12] | 258 |
| 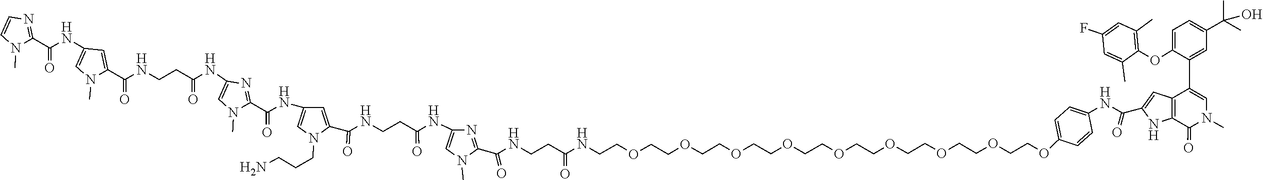 [SMILES: Cc1cc(F)cc(C)c1Oc1ccc(C(C)(C)O)cc1-c1cn(C)c(=O)c2[nH]c(C(=O)Nc3ccc(OCCOCCOCCOCCOCCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5CCCN)cn4C)cc3)cc12] | 259 |
| 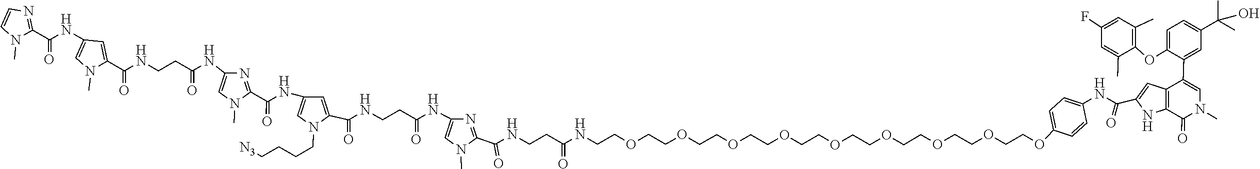 [SMILES: Cc1cc(F)cc(C)c1Oc1ccc(C(C)(C)O)cc1-c1cn(C)c(=O)c2[nH]c(C(=O)Nc3ccc(OCCOCCOCCOCCOCCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5CCCCN=[N+]=[N-])cn4C)cc3)cc12] | 260 |
| 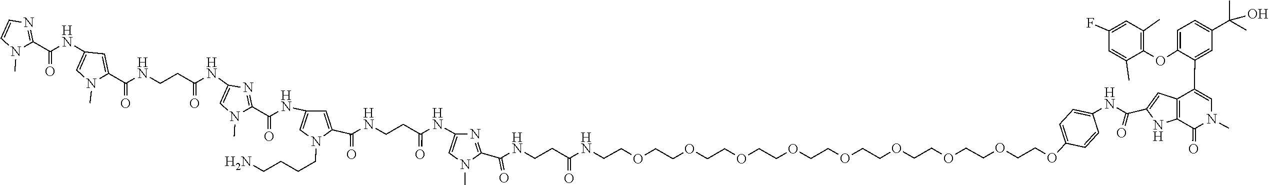 | 261 |
| 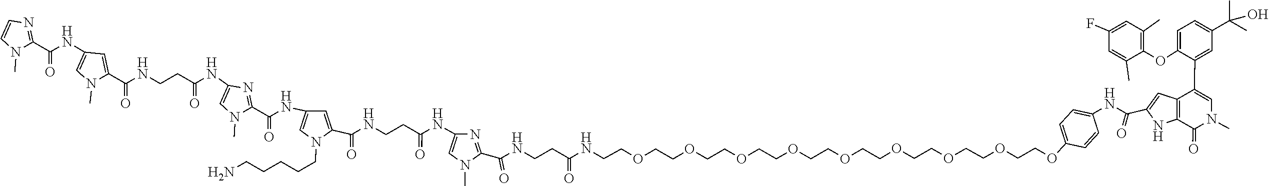 | 262 |
| 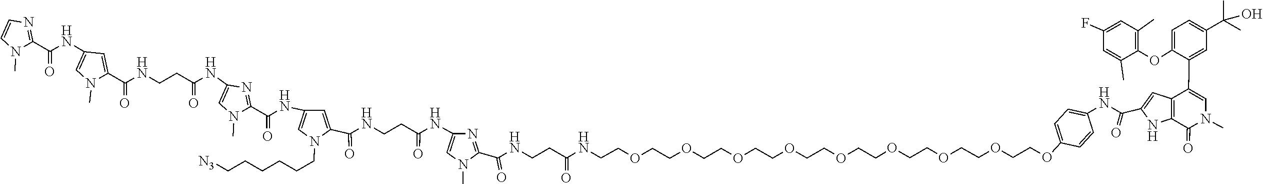 [SMILES: Cc1cc(F)cc(C)c1Oc1ccc(C(C)(C)O)cc1-c1cn(C)c(=O)c2[nH]c(C(=O)Nc3ccc(OCCOCCOCCOCCOCCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5CCCCCCN=[N+]=[N-])cn4C)cc3)cc12] | 263 |
| 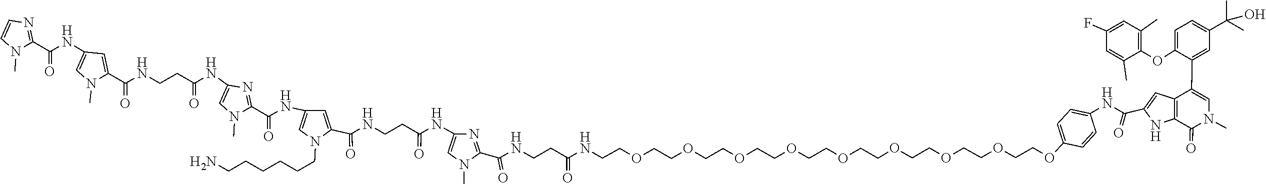 [SMILES: Cc1cc(F)cc(C)c1Oc1ccc(C(C)(C)O)cc1-c1cn(C)c(=O)c2[nH]c(C(=O)Nc3ccc(OCCOCCOCCOCCOCCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5CCCCCCN)cn4C)cc3)cc12] | 264 |
| 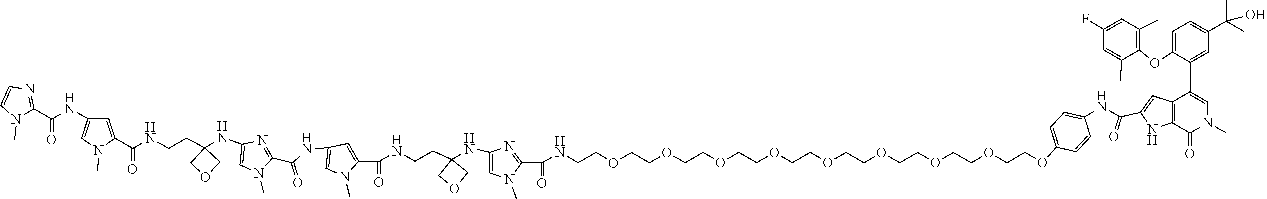 [SMILES: Cc1cc(F)cc(C)c1Oc1ccc(C(C)(C)O)cc1-c1cn(C)c(=O)c2[nH]c(C(=O)Nc3ccc(OCCOCCOCCOCCOCCOCCOCCOCCOCCNC(=O)c4nc(NC5(CCNC(=O)c6cc(NC(=O)c7nc(NC8(CCNC(=O)c9cc(NC(=O)c%10nccn%10C)cn9C)COC8)cn7C)cn6C)COC5)cn4C)cc3)cc12] | 265 |

### Methods of Use

The present disclosure also relates to a method of modulating the transcription of fxn comprising the step of contacting fxn with a compound as described herein. The cell phenotype, cell proliferation, transcription of fxn, production of mRNA from transcription of fxn, translation of fxn, change in biochemical output produced by the protein coded by fxn, or noncovalent binding of the protein coded by fxn with a natural binding partner may be monitored. Such methods may be modes of treatment of disease, biological assays, cellular assays, biochemical assays, or the like.

Also provided herein is a method of treating of a disease mediated by transcription of fxn comprising administering a therapeutically effective amount of a transcription modulator molecule as disclosed herein, or a salt thereof, to a patient in need thereof.

In certain embodiments, the disease is Friedreich's ataxia (FA).

Also provided is the use of a transcription modulator molecule as disclosed herein as a medicament for the treatment of a disease mediated by transcription of fxn.

Also provided herein is a method of modulation of transcription of fxn comprising contacting fxn with a transcription modulator molecule as disclosed herein, or a salt thereof.

Also provided herein is a method of treating Friedreich's ataxia in a patient in need thereof, comprising administering to the patient a transcription modulator molecule as described herein.

Also provided herein is a method for achieving an effect in a patient comprising the administration of a therapeutically effective amount of a compound as disclosed herein, or a salt thereof, to a patient, wherein the effect is chosen from improved neural sensation, improved vision, improved balance, improved gait, reduced sensitivity to glucose, and reduced sensitivity to carbohydrates.

In some embodiments, the method comprises alleviating one or more of muscular atrophy, ataxia, fasciculation, or dementia.

Certain compounds of the present disclosure may be effective for treatment of subjects whose genotype has 5 or more repeats of GAA. Certain compounds of the present disclosure may be effective for treatment of subjects whose genotype has 10 or more repeats of GAA. Certain compounds of the present disclosure may be effective for treatment of subjects whose genotype has 20 or more repeats of GAA. Certain compounds of the present disclosure may be effective for treatment of subjects whose genotype has 50 or more repeats of GAA. Certain compounds of the present disclosure may be effective for treatment of subjects whose genotype has 100 or more repeats of GAA. Certain compounds of the present disclosure may be effective for treatment of subjects whose genotype has 200 or more repeats of GAA. Certain compounds of the present disclosure may be effective for treatment of subjects whose genotype has 500 or more repeats of GAA.

Also provided is a method of modulation of a fxn-mediated function in a subject comprising the administration of a therapeutically effective amount of a compound as disclosed herein.

Also provided is a pharmaceutical composition comprising a compound as disclosed herein, together with a pharmaceutically acceptable carrier.

In certain embodiments, the pharmaceutical composition is formulated for oral administration.

In certain embodiments, the pharmaceutical composition is formulated for intravenous injection and/or infusion.

In certain embodiments, the oral pharmaceutical composition is chosen from a tablet and a capsule.

In certain embodiments, ex vivo methods of treatment are provided. Ex vivo methods typically include cells, organs, and/or tissues removed from the subject. The cells, organs and/or tissues can, for example, be incubated with the agent under appropriate conditions. The contacted cells, organs, and/or tissues are typically returned to the donor, placed in a recipient, or stored for future use. Thus, the compound is generally in a pharmaceutically acceptable carrier.

In certain embodiments, administration of the pharmaceutical composition modulates expression of fxn within 6 hours of treatment. In certain embodiments, administration of the pharmaceutical composition modulates expression of fxn within 24 hours of treatment. In certain embodiments, administration of the pharmaceutical composition modulates expression of fxn within 72 hours of treatment.

In certain embodiments, administration of the pharmaceutical composition causes a 2-fold increase in expression of fxn. In certain embodiments, administration of the pharmaceutical composition causes a 5-fold increase in expression of fxn. In certain embodiments, administration of the pharmaceutical composition causes a 10-fold increase in expression of fxn. In certain embodiments, administration of the pharmaceutical composition causes a 20-fold increase in expression of fxn.

In certain embodiments, administration of the pharmaceutical composition causes a 20% decrease in expression of fxn. In certain embodiments, administration of the pharmaceutical composition causes a 50% decrease in expression of fxn. In certain embodiments, administration of the pharmaceutical composition causes a 80% decrease in expression of fxn. In certain embodiments, administration of the pharmaceutical composition causes a 90% decrease in expression of fxn. In certain embodiments, administration of the pharmaceutical composition causes a 95% decrease in expression of fxn. In certain embodiments, administration of the pharmaceutical composition causes a 99% decrease in expression of fxn.

In certain embodiments, administration of the pharmaceutical composition causes expression of fxn to fall within 25% of the level of expression observed for healthy individuals. In certain embodiments, administration of the pharmaceutical composition causes expression of fxn to fall within 50% of the level of expression observed for healthy individuals. In certain embodiments, administration of the pharmaceutical composition causes expression of fxn to fall within 75% of the level of expression observed for healthy individuals. In certain embodiments, administration of the pharmaceutical composition causes expression of fxn to fall within 90% of the level of expression observed for healthy individuals.

### Pharmaceutical Compositions and Administration

Also provided is a method of modulation of a fxn-mediated function in a subject comprising the administration of a therapeutically effective amount of a compound as disclosed herein.

Also provided is a pharmaceutical composition comprising a compound as disclosed herein, together with a pharmaceutically acceptable carrier.

In certain embodiments, the pharmaceutical composition is formulated for oral administration.

In certain embodiments, the pharmaceutical composition is formulated for intravenous injection or infusion.

In certain embodiments, the oral pharmaceutical composition is chosen from a tablet and a capsule.

In certain embodiments, ex vivo methods of treatment are provided. Ex vivo methods typically include cells, organs, or tissues removed from the subject. The cells, organs or tissues can, for example, be incubated with the agent under appropriate conditions. The contacted cells, organs, or tissues are typically returned to the donor, placed in a recipient, or stored for future use. Thus, the compound is generally in a pharmaceutically acceptable carrier.

In certain embodiments, the compound is effective at a concentration less than about 5 μM. In certain embodiments, the compound is effective at a concentration less than about 1 μM. In certain embodiments, the compound is effective at a concentration less than about 400 nM. In certain embodiments, the compound is effective at a concentration less than about 200 nM. In certain embodiments, the compound is effective at a concentration less than about 100 nM. In certain embodiments, the compound is effective at a concentration less than about 50 nM. In certain embodiments, the compound is effective at a concentration less than about 20 nM. In certain embodiments, the compound is effective at a concentration less than about 10 nM.

### Abbreviations and Definitions

As used herein, the terms below have the meanings indicated.

It is to be understood that certain radical naming conventions can include either a mono-radical or a di-radical, depending on the context. For example, where a substituent requires two points of attachment to the rest of the molecule, it is understood that the substituent is a di-radical. For example, a substituent identified as alkyl that requires two points of attachment includes di-radicals such as —CH₂—, —CH₂CH₂—, —CH₂CH(CH₃)CH₂—, and the like. Other radical naming conventions clearly indicate that the radical is a di-radical such as “alkylene,” “alkenylene,” “arylene”, “heteroarylene.”

When two R groups are said to form a ring (e.g., a carbocyclyl, heterocyclyl, aryl, or heteroaryl ring) “together with the atom to which they are attached,” it is meant that the collective unit of the atom and the two R groups are the recited ring. The ring is not otherwise limited by the definition of each R group when taken individually. For example, when the following substructure is present:

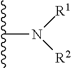

and R¹ and R² are defined as selected from the group consisting of hydrogen and alkyl, or R¹ and R² together with the nitrogen to which they are attached form a heterocyclyl, it is meant that R¹ and R² can be selected from hydrogen or alkyl, or alternatively, the substructure has structure:

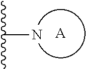

where ring A is a heteroaryl ring containing the depicted nitrogen.

Similarly, when two “adjacent” R groups are said to form a ring “together with the atom to which they are attached,” it is meant that the collective unit of the atoms, intervening bonds, and the two R groups are the recited ring. For example, when the following substructure is present:

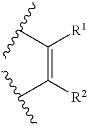

and R¹ and R² are defined as selected from the group consisting of hydrogen and alkyl, or R¹ and R² together with the atoms to which they are attached form an aryl or carbocylyl, it is meant that R¹ and R² can be selected from hydrogen or alkyl, or alternatively, the substructure has structure:

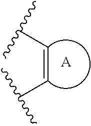

where A is an aryl ring or a carbocylyl containing the depicted double bond.

Wherever a substituent is depicted as a di-radical (i.e., has two points of attachment to the rest of the molecule), it is to be understood that the substituent can be attached in any directional configuration

unless otherwise indicated. Thus, for example, a substituent depicted as -AE- or

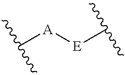

includes the substituent being oriented such that the A is attached at the leftmost attachment point of the molecule as well as the case in which A is attached at the rightmost attachment point of the molecule.

When ranges of values are disclosed, and the notation “from n₁ . . . to n₂” or “between n₁ . . . and n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

The term “polyamide” refers to polymers of linkable units chemically bound by amide (i.e., CONH) linkages; optionally, polyamides include chemical probes conjugated therewith. Polyamides may be synthesized by stepwise condensation of carboxylic acids (COOH) with amines (RR′NH) using methods known in the art. Alternatively, polyamides may be formed using enzymatic reactions in vitro, or by employing fermentation with microorganisms.

The term “linkable unit” refers to methylimidazoles, methylpyrroles, and straight and branched chain aliphatic functionalities (e.g., methylene, ethylene, propylene, butylene, and the like) which optionally contain nitrogen Substituents, and chemical derivatives thereof. The aliphatic functionalities of linkable units can be provided, for example, by condensation of B-alanine or dimethylaminopropylamine during synthesis of the polyamide by methods well known in the art.

The term “linker” refers to a chain of at least 10 contiguous atoms. In certain embodiments, the linker contains no more than 20 non-hydrogen atoms. In certain embodiments, the linker contains no more than 40 non-hydrogen atoms. In certain embodiments, the linker contains no more than 60 non-hydrogen atoms. In certain embodiments, the linker contains atoms chosen from C, H, N, O, and S. In certain embodiments, every non-hydrogen atom is chemically bonded either to 2 neighboring atoms in the linker, or one neighboring atom in the linker and a terminus of the linker. In certain embodiments, the linker forms an amide bond with at least one of the two other groups to which it is attached. In certain embodiments, the linker forms an ester or ether bond with at least one of the two other groups to which it is attached. In certain embodiments, the linker forms a thioester or thioether bond with at least one of the two other groups to which it is attached. In certain embodiments, the linker forms a direct carbon-carbon bond with at least one of the two other groups to which it is attached. In certain embodiments, the linker forms an amine or amide bond with at least one of the two other groups to which it is attached. In certain embodiments, the linker comprises —(CH₂OCH₂)— units. In certain embodiments, the linker comprises —(CH(CH₃)OCH₂)— units. In certain embodiments, the linker comprises —(CH₂NR_{N}CH₂) units, for R_{N}═C_{M4}alkyl. In certain embodiments, the linker comprises an arylene, cycloalkylene, or heterocycloalkylene moiety.

The term “spacer” refers to a chain of at least 5 contiguous atoms. In certain embodiments, the spacer contains no more than 10 non-hydrogen atoms. In certain embodiments, the spacer contains atoms chosen from C, H, N, O, and S. In certain embodiments, the spacer forms amide bonds with the two other groups to which it is attached. In certain embodiments, the spacer comprises —(CH₂OCH₂)— units. In certain embodiments, the spacer comprises —(CH₂NR_{N}CH₂)— units, for R_{N}═C₁₋₄alkyl. In certain embodiments, the spacer contains at least one positive charge at physiological pH.

The term “turn component” refers to a chain of about 4 to 10 contiguous atoms. In certain embodiments, the turn component contains atoms chosen from C, H, N, O, and S. In certain embodiments, the turn component forms amide bonds with the two other groups to which it is attached. In certain embodiments, the turn component contains at least one positive charge at physiological pH.

The terms “nucleic acid and “nucleotide” refer to ribonucleotide and deoxyribonucleotide, and analogs thereof, well known in the art.

The term “oligonucleotide sequence” refers to a plurality of nucleic acids having a defined sequence and length (e.g., 2, 3, 4, 5, 6, or even more nucleotides). The term “oligonucleotide repeat sequence” refers to a contiguous expansion of oligonucleotide sequences.

The term “transcription,” well known in the art, refers to the synthesis of RNA (i.e., ribonucleic acid) by DNA-directed RNA polymerase. The term “modulate transcription” refers to a change in transcriptional level which can be measured by methods well known in the art, for example, assay of mRNA, the product of transcription. In certain embodiments, modulation is an increase in transcription. In other embodiments, modulation is a decrease in transcription.

The term “contacting” refers to bringing the compound (e.g. a transcription molecular molecule of the present disclosure) into proximity of the desired target gene. The contacting may result in the binding to or result in a conformational change of the target moiety.

The term “acyl,” as used herein, alone or in combination, refers to a carbonyl attached to an alkenyl, alkyl, aryl, cycloalkyl, heteroaryl, heterocycle, or any other moiety were the atom attached to the carbonyl is carbon. An “acetyl” group refers to a —C(O)CH₃ group. An “alkylcarbonyl” or “alkanoyl” group refers to an alkyl group attached to the parent molecular moiety through a carbonyl group. Examples of such groups include methylcarbonyl and ethylcarbonyl. Examples of acyl groups include formyl, alkanoyl and aroyl.

The term “alkenyl,” as used herein, alone or in combination, refers to a straight-chain or branched-chain hydrocarbon radical having one or more double bonds and containing from 2 to 20 carbon atoms. In certain embodiments, said alkenyl will comprise from 2 to 6 carbon atoms. The term “alkenylene” refers to a carbon-carbon double bond system attached at two or more positions such as ethenylene [(—CH═CH—),(—C::C—)]. Examples of suitable alkenyl radicals include ethenyl, propenyl, 2-methylpropenyl, 1,4-butadienyl and the like. Unless otherwise specified, the term “alkenyl” may include “alkenylene” groups.

The term “alkoxy,” as used herein, alone or in combination, refers to an alkyl ether radical, wherein the term alkyl is as defined below. Examples of suitable alkyl ether radicals include methoxy, ethoxy, n-propoxy, isopropoxy, n-butoxy, iso-butoxy, sec-butoxy, tert-butoxy, and the like.

The term “alkyl,” as used herein, alone or in combination, refers to a straight-chain or branched-chain alkyl radical containing from 1 to 20 carbon atoms. In certain embodiments, said alkyl will comprise from 1 to 10 carbon atoms. In further embodiments, said alkyl will comprise from 1 to 8 carbon atoms. Alkyl groups may be optionally substituted as defined herein. Examples of alkyl radicals include methyl, ethyl, n-propyl, isopropyl, n-butyl, isobutyl, sec-butyl, tert-butyl, pentyl, iso-amyl, hexyl, octyl, noyl and the like. The term “alkylene,” as used herein, alone or in combination, refers to a saturated aliphatic group derived from a straight or branched chain saturated hydrocarbon attached at two or more positions, such as methylene (—CH₂—). Unless otherwise specified, the term “alkyl” may include “alkylene” groups.

The term “alkylamino,” as used herein, alone or in combination, refers to an alkyl group attached to the parent molecular moiety through an amino group. Suitable alkylamino groups may be mono- or dialkylated, forming groups such as, for example, N-methylamino, N-ethylamino, N,N-dimethylamino, N,N-ethylmethylamino and the like.

The term “alkylidene,” as used herein, alone or in combination, refers to an alkenyl group in which one carbon atom of the carbon-carbon double bond belongs to the moiety to which the alkenyl group is attached.

The term “alkylthio,” as used herein, alone or in combination, refers to an alkyl thioether (R—S—) radical wherein the term alkyl is as defined above and wherein the sulfur may be singly or doubly oxidized.

Examples of suitable alkyl thioether radicals include methylthio, ethylthio, n-propylthio, isopropylthio, n-butylthio, iso-butylthio, sec-butylthio, tert-butylthio, methanesulfonyl, ethanesulfinyl, and the like.

The term “alkynyl,” as used herein, alone or in combination, refers to a straight-chain or branched chain hydrocarbon radical having one or more triple bonds and containing from 2 to 20 carbon atoms. In certain embodiments, said alkynyl comprises from 2 to 6 carbon atoms. In further embodiments, said alkynyl comprises from 2 to 4 carbon atoms. The term “alkynylene” refers to a carbon-carbon triple bond attached at two positions such as ethynylene [(—C:::C—, —C—C—)]. Examples of alkynyl radicals include ethynyl, propynyl, hydroxypropynyl, butyn-1-yl, butyn-2-yl, pentyn-1-yl, 3-methylbutyn-1-yl, hexyn-2-yl, and the like. Unless otherwise specified, the term “alkynyl” may include “alkynylene” groups.

The terms “amido” and “carbamoyl,” as used herein, alone or in combination, refer to an amino group as described below attached to the parent molecular moiety through a carbonyl group, or vice versa.

The term “C-amido” as used herein, alone or in combination, refers to a —C(O)N(RR′) group with R and R′ as defined herein or as defined by the specifically enumerated “R” groups designated. The term “N-amido” as used herein, alone or in combination, refers to a RC(O)N(R′)— group, with R and R′ as defined herein or as defined by the specifically enumerated “R” groups designated. The term “acylamino” as used herein, alone or in combination, embraces an acyl group attached to the parent moiety through an amino group. An example of an “acylamino” group is acetylamino (CH₃C(O)NH—).

The term “amide,” as used herein, alone in combination, refers to —C(O)NRR′, wherein R and R′ are independently chosen from hydrogen, alkyl, acyl, heteroalkyl, aryl, cycloalkyl, heteroaryl, and heterocycloalkyl, any of which may themselves be optionally substituted. Additionally, R and R′ may combine to form heterocycloalkyl, either of which may be optionally substituted. Amides may be formed by direct condensation of carboxylic acids with amines, or by using acid chlorides. In addition, coupling reagents are known in the art, including carbodiimide-based compounds such as DCC and EDCI.

The term “amino,” as used herein, alone or in combination, refers to —NRR′, wherein R and R′ are independently chosen from hydrogen, alkyl, acyl, heteroalkyl, aryl, cycloalkyl, heteroaryl, and heterocycloalkyl, any of which may themselves be optionally substituted. Additionally, R and R′ may combine to form heterocycloalkyl, either of which may be optionally substituted.

The term “aryl,” as used herein, alone or in combination, means a carbocyclic aromatic system containing one, two or three rings wherein such polycyclic ring systems are fused together. The term “aryl” embraces aromatic groups such as phenyl, naphthyl, anthracenyl, and phenanthryl. The term “arylene” embraces aromatic groups such as phenylene, naphthylene, anthracenylene, and phenanthrylene.

The term “arylalkenyl” or “aralkenyl,” as used herein, alone or in combination, refers to an aryl group attached to the parent molecular moiety through an alkenyl group.

The term “arylalkoxy” or “aralkoxy,” as used herein, alone or in combination, refers to an aryl group attached to the parent molecular moiety through an alkoxy group.

The term “arylalkyl” or “aralkyl,” as used herein, alone or in combination, refers to an aryl group attached to the parent molecular moiety through an alkyl group.

The term “arylalkynyl” or “aralkynyl,” as used herein, alone or in combination, refers to an aryl group attached to the parent molecular moiety through an alkynyl group.

The term “arylalkanoyl” or “aralkanoyl” or “aroyl,” as used herein, alone or in combination, refers to an acyl radical derived from an aryl-substituted alkanecarboxylic acid such as benzoyl, napthoyl, phenylacetyl, 3-phenylpropionyl (hydrocinnamoyl), 4-phenylbutyryl, (2-naphthyl)acetyl, 4-chlorohydrocinnamoyl, and the like.

The term aryloxy as used herein, alone or in combination, refers to an aryl group attached to the parent molecular moiety through an oxy.

The terms “benzo” and “benz,” as used herein, alone or in combination, refer to the divalent radical C₆H₄═ derived from benzene. Examples include benzothiophene and benzimidazole.

The term “carbamate,” as used herein, alone or in combination, refers to an ester of carbamic acid (—NHCOO-) which may be attached to the parent molecular moiety from either the nitrogen or acid end, and which may be optionally substituted as defined herein.

The term “O-carbamyl” as used herein, alone or in combination, refers to a —OC(O)NRR′, group-with R and R′ as defined herein.

The term “N-carbamyl” as used herein, alone or in combination, refers to a ROC(O)NR′— group, with R and R′ as defined herein.

The term “carbonyl,” as used herein, when alone includes formyl [—C(O)H] and in combination is a —C(O)— group.

The term “carboxyl” or “carboxy,” as used herein, refers to —C(O)OH or the corresponding “carboxylate” anion, such as is in a carboxylic acid salt. An “O-carboxy” group refers to a RC(O)O— group, where R is as defined herein. A “C-carboxy” group refers to a —C(O)OR groups where R is as defined herein.

The term “cyano,” as used herein, alone or in combination, refers to —CN.

The term “cycloalkyl,” or, alternatively, “carbocycle,” as used herein, alone or in combination, refers to a saturated or partially saturated monocyclic, bicyclic or tricyclic alkyl group wherein each cyclic moiety contains from 3 to 12 carbon atom ring members and which may optionally be a benzo fused ring system which is optionally substituted as defined herein. In certain embodiments, said cycloalkyl will comprise from 5 to 7 carbon atoms. Examples of such cycloalkyl groups include cyclopropyl, cyclobutyl, cyclopentyl, cyclohexyl, cycloheptyl, tetrahydronapthyl, indanyl, octahydronaphthyl, 2,3-dihydro-1H-indenyl, adamantyl and the like. “Bicyclic” and “tricyclic” as used herein are intended to include both fused ring systems, such as decahydronaphthalene, octahydronaphthalene as well as the multicyclic (multicentered) saturated or partially unsaturated type. The latter type of isomer is exemplified in general by, bicyclo[1,1,1]pentane, camphor, adamantane, and bicyclo[3,2,1]octane.

The term “ester,” as used herein, alone or in combination, refers to a carboxy group bridging two moieties linked at carbon atoms.

The term “ether,” as used herein, alone or in combination, refers to an oxy group bridging two moieties linked at carbon atoms.

The term “halo,” or “halogen,” as used herein, alone or in combination, refers to fluorine, chlorine, bromine, or iodine.

The term “haloalkoxy,” as used herein, alone or in combination, refers to a haloalkyl group attached to the parent molecular moiety through an oxygen atom.

The term “haloalkyl,” as used herein, alone or in combination, refers to an alkyl radical having the meaning as defined above wherein one or more hydrogens are replaced with a halogen. Specifically embraced are monohaloalkyl, dihaloalkyl and polyhaloalkyl radicals. A monohaloalkyl radical, for one example, may have an iodo, bromo, chloro or fluoro atom within the radical. Dihalo and polyhaloalkyl radicals may have two or more of the same halo atoms or a combination of different halo radicals. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a haloalkyl group attached at two or more positions. Examples include fluoromethylene (—CFH—), difluoromethylene (—CF₂—), chloromethylene (—CHCl—) and the like.

The term “heteroalkyl,” as used herein, alone or in combination, refers to a stable straight or branched chain, or combinations thereof, fully saturated or containing from 1 to 3 degrees of unsaturation, consisting of the stated number of carbon atoms and from one to three heteroatoms chosen from N, O, and S, and wherein the N and S atoms may optionally be oxidized and the N heteroatom may optionally be quaternized. The heteroatom(s) may be placed at any interior position of the heteroalkyl group. Up to two heteroatoms may be consecutive, such as, for example, —CH₂—NH—OCH₃.

The term “heteroaryl,” as used herein, alone or in combination, refers to a 3 to 15 membered unsaturated heteromonocyclic ring, or a fused monocyclic, bicyclic, or tricyclic ring system in which at least one of the fused rings is aromatic, which contains at least one atom chosen from N, O, and S. In certain embodiments, said heteroaryl will comprise from 1 to 4 heteroatoms as ring members. In further embodiments, said heteroaryl will comprise from 1 to 2 heteroatoms as ring members. In certain embodiments, said heteroaryl will comprise from 5 to 7 atoms. The term also embraces fused polycyclic groups wherein heterocyclic rings are fused with aryl rings, wherein heteroaryl rings are fused with other heteroaryl rings, wherein heteroaryl rings are fused with heterocycloalkyl rings, or wherein heteroaryl rings are fused with cycloalkyl rings. Examples of heteroaryl groups include pyrrolyl, pyrrolinyl, imidazolyl, pyrazolyl, pyridyl, pyrimidinyl, pyrazinyl, pyridazinyl, triazolyl, pyranyl, furyl, thienyl, oxazolyl, isoxazolyl, oxadiazolyl, thiazolyl, thiadiazolyl, isothiazolyl, indolyl, isoindolyl, indolizinyl, benzimidazolyl, quinolyl, isoquinolyl, quinoxalinyl, quinazolinyl, indazolyl, benzotriazolyl, benzodioxolyl, benzopyranyl, benzoxazolyl, benzoxadiazolyl, benzothiazolyl, benzothiadiazolyl, benzofuryl, benzothienyl, chromonyl, coumarinyl, benzopyranyl, tetrahydroquinolinyl, tetrazolopyridazinyl, tetrahydroisoquinolinyl, thienopyridinyl, furopyridinyl, pyrrolopyridinyl and the like. Exemplary tricyclic heterocyclic groups include carbazolyl, benzidolyl, phenanthrolinyl, dibenzofuranyl, acridinyl, phenanthridinyl, xanthenyl and the like.

The terms “heterocycloalkyl” and, interchangeably, “heterocycle,” as used herein, alone or in combination, each refer to a saturated, partially unsaturated, or fully unsaturated (but nonaromatic) monocyclic, bicyclic, or tricyclic heterocyclic group containing at least one heteroatom as a ring member, wherein each said heteroatom may be independently chosen from nitrogen, oxygen, and sulfur. In certain embodiments, said heterocycloalkyl will comprise from 1 to 4 heteroatoms as ring members. In further embodiments, said heterocycloalkyl will comprise from 1 to 2 heteroatoms as ring members. In certain embodiments, said heterocycloalkyl will comprise from 3 to 8 ring members in each ring. In further embodiments, said heterocycloalkyl will comprise from 3 to 7 ring members in each ring. In yet further embodiments, said heterocycloalkyl will comprise from 5 to 6 ring members in each ring. “Heterocycloalkyl” and “heterocycle” are intended to include sulfones, sulfoxides, N-oxides of tertiary nitrogen ring members, and carbocyclic fused and benzo fused ring systems; additionally, both terms also include systems where a heterocycle ring is fused to an aryl group, as defined herein, or an additional heterocycle group. Examples of heterocycle groups include tetrhydroisoquinoline, aziridinyl, azetidinyl, 1,3-benzodioxolyl, dihydroisoindolyl, dihydroisoquinolinyl, dihydrocinnolinyl, dihydrobenzodioxinyl, dihydro[1,3]oxazolo[4,5-b]pyridinyl, benzothiazolyl, dihydroindolyl, dihy-dropyridinyl, 1,3-dioxanyl, 1,4-dioxanyl, 1,3-dioxolanyl, isoindolinyl, morpholinyl, piperazinyl, pyrrolidinyl, tetrahydropyridinyl, piperidinyl, thiomorpholinyl, and the like. The heterocycle groups may be optionally substituted unless specifically prohibited.

The term “hydrazinyl” as used herein, alone or in combination, refers to two amino groups joined by a single bond, i.e., —N—N—.

The term “hydroxy,” as used herein, alone or in combination, refers to —OH.

The term “hydroxyalkyl,” as used herein, alone or in combination, refers to a hydroxy group attached to the parent molecular moiety through an alkyl group.

The term “imino,” as used herein, alone or in combination, refers to ═N—.

The term “iminohydroxy,” as used herein, alone or in combination, refers to ═N(OH) and ═N—O—.

The phrase “in the main chain” refers to the longest contiguous or adjacent chain of carbon atoms starting at the point of attachment of a group to the compounds or molecules of any one of the formulas disclosed herein.

The term “isocyanato” refers to a —NCO group.

The term “isothiocyanato” refers to a —NCS group.

The phrase “linear chain of atoms” refers to the longest straight chain of atoms independently selected from carbon, nitrogen, oxygen and sulfur.

The term “lower,” as used herein, alone or in a combination, where not otherwise specifically defined, means containing from 1 to and including 6 carbon atoms (i.e., C₁-C₆ alkyl).

The term “lower aryl,” as used herein, alone or in combination, means phenyl or naphthyl, either of which may be optionally substituted as provided.

The term “lower heteroaryl,” as used herein, alone or in combination, means either 1) monocyclic heteroaryl comprising five or six ring members, of which between one and four said members may be heteroatoms chosen from N, O, and S, or 2) bicyclic heteroaryl, wherein each of the fused rings comprises five or six ring members, comprising between them one to four heteroatoms chosen from N, O, and S.

The term “lower cycloalkyl,” as used herein, alone or in combination, means a monocyclic cycloalkyl having between three and six ring members (i.e., C₃-C₆ cycloalkyl). Lower cycloalkyls may be unsaturated. Examples of lower cycloalkyl include cyclopropyl, cyclobutyl, cyclopentyl, and cyclohexyl.

The term “lower heterocycloalkyl,” as used herein, alone or in combination, means a monocyclic heterocycloalkyl having between three and six ring members, of which between one and four may be heteroatoms chosen from N, O, and S (i.e., C₃-C₆ heterocycloalkyl). Examples of lower heterocycloalkyls include pyrrolidinyl, imidazolidinyl, pyrazolidinyl, piperidinyl, piperazinyl, and morpholinyl. Lower heterocycloalkyls may be unsaturated.

The term “lower amino,” as used herein, alone or in combination, refers to —NRR′, wherein R and R′ are independently chosen from hydrogen and lower alkyl, either of which may be optionally substituted.

The term “mercaptyl” as used herein, alone or in combination, refers to an RS— group, where R is as defined herein.

The term “nitro,” as used herein, alone or in combination, refers to —NO₂.

The terms “oxy” or “oxa,” as used herein, alone or in combination, refer to —O—.

The term “oxo,” as used herein, alone or in combination, refers to ═O.

The term “perhaloalkoxy” refers to an alkoxy group where all of the hydrogen atoms are replaced by halogen atoms.

The term “perhaloalkyl” as used herein, alone or in combination, refers to an alkyl group where all of the hydrogen atoms are replaced by halogen atoms.

The terms “sulfonate,” “sulfonic acid,” and “sulfonic,” as used herein, alone or in combination, refer the —SO₃H group and its anion as the sulfonic acid is used in salt formation.

The term “sulfanyl,” as used herein, alone or in combination, refers to —S—.

The term “sulfinyl,” as used herein, alone or in combination, refers to —S(O)—.

The term “sulfonyl,” as used herein, alone or in combination, refers to —S(O)₂—.

The term “N-sulfonamido” refers to a RS(═O)₂NR′— group with R and R′ as defined herein.

The term “S-sulfonamido” refers to a —S(═O)₂NRR′, group, with R and R′ as defined herein.

The terms “thia” and “thio,” as used herein, alone or in combination, refer to a —S— group or an ether wherein the oxygen is replaced with sulfur. The oxidized derivatives of the thio group, namely sulfinyl and sulfonyl, are included in the definition of thia and thio.

The term “thiol,” as used herein, alone or in combination, refers to an —SH group.

The term “thiocarbonyl,” as used herein, when alone includes thioformyl —C(S)H and in combination is a —C(S)— group.

The term “N-thiocarbamyl” refers to an ROC(S)NR′ group, with R and R′ as defined herein.

The term “O-thiocarbamyl” refers to a OC(S)NRR′, group with R and R′ as defined herein.

The term “thiocyanato” refers to a CNS group.

The term “trihalomethanesulfonamido” refers to a X₃CS(O)₂NR group with X is a halogen and R as defined herein.

The term “trihalomethanesulfonyl” refers to a X₃CS(O)₂ group where X is a halogen.

The term “trihalomethoxy” refers to a X₃CO group where X is a halogen.

The term “trisubstituted silyl,” as used herein, alone or in combination, refers to a silicone group substituted at its three free valences with groups as listed herein under the definition of substituted amino. Examples include trimethysilyl, tert-butyldimethylsilyl, triphenylsilyl and the like.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

The term “optionally substituted” means the anteceding group may be substituted or unsubstituted.

When substituted, the substituents of an “optionally substituted” group may include, without limitation, one or more substituents independently selected from the following groups or a particular designated set of groups, alone or in combination: lower alkyl, lower alkenyl, lower alkynyl, lower alkanoyl, lower heteroalkyl, lower heterocycloalkyl, lower haloalkyl, lower haloalkenyl, lower haloalkynyl, lower perhaloalkyl, lower perhaloalkoxy, lower cycloalkyl, phenyl, aryl, aryloxy, lower alkoxy, lower haloalkoxy, oxo, lower acyloxy, carbonyl, carboxyl, lower alkylcarbonyl, lower carboxyester, lower carboxamido, cyano, hydrogen, halogen, hydroxy, amino, lower alkylamino, arylamino, amido, nitro, thiol, lower alkylthio, lower haloalkylthio, lower perhaloalkylthio, arylthio, sulfonate, sulfonic acid, trisubstituted silyl, N₃, SH, SCH₃, C(O)CH₃, CO₂CH₃, CO₂H, pyridinyl, thiophene, furanyl, lower carbamate, and lower urea.

Where structurally feasible, two substituents may be joined together to form a fused five-, six-, or seven-membered carbocyclic or heterocyclic ring consisting of zero to three heteroatoms, for example forming methylenedioxy or ethylenedioxy. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃). Where substituents are recited without qualification as to substitution, both substituted and unsubstituted forms are encompassed. Where a substituent is qualified as “substituted,” the substituted form is specifically intended. Additionally, different sets of optional substituents to a particular moiety may be defined as needed; in these cases, the optional substitution will be as defined, often immediately following the phrase, “optionally substituted with”.

As used herein, a substituted group is derived from the unsubstituted parent group in which there has been an exchange of one or more hydrogen atoms for another atom or group. Unless otherwise indicated, when a group is deemed to be “substituted,” it is meant that the group is substituted with one or more substituents independently selected from C₁-C₆ alkyl, C₁-C₆ alkenyl, C₁-C₆ alkynyl, C₁-C₆ heteroalkyl, C₃-C₇ carbocyclyl (optionally substituted with halo, C₁-C₆ alkyl, C₁-C₆ alkoxy, C₁-C₆ haloalkyl, and C₁-C₆ haloalkoxy), C₃-C₇-carbocyclyl-C₁-C₆-alkyl (optionally substituted with halo, C₁-C₆ alkyl, C₁-C₆ alkoxy, C₁-C₆ haloalkyl, and C₁-C₆ haloalkoxy), 3-10 membered heterocyclyl (optionally substituted with halo, C₁-C₆ alkyl, C₁-C₆ alkoxy, C₁-C₆ haloalkyl, and C₁-C₆ haloalkoxy), 3-10 membered heterocyclyl-C₁-C₆-alkyl (optionally substituted with halo, C₁-C₆ alkyl, C₁-C₆ alkoxy, C₁-C₆ haloalkyl, and C₁-C₆ haloalkoxy), aryl (optionally substituted with halo, C₁-C₆ alkyl, C₁-C₆ alkoxy, C₁-C₆ haloalkyl, and C₁-C₆ haloalkoxy), aryl(C₁-C₆)alkyl (optionally substituted with halo, C₁-C₆ alkyl, C₁-C₆ alkoxy, C₁-C₆ haloalkyl, and C₁-C₆ haloalkoxy), 5-10 membered heteroaryl (optionally substituted with halo, C₁-C₆ alkyl, C₁-C₆ alkoxy, C₁-C₆ haloalkyl, and C₁-C₆ haloalkoxy), 5-10 membered heteroaryl(C₁-C₆)alkyl (optionally substituted with halo, C₁-C₆ alkyl, C₁-C₆ alkoxy, C₁-C₆ haloalkyl, and C₁-C₆ haloalkoxy), halo, cyano, hydroxy, C₁-C₆ alkoxy, C₁-C₆ alkoxy(C₁-C₆)alkyl (i.e., ether), aryloxy, sulfhydryl (mercapto), halo(C₁-C₆)alkyl (e.g., —CF₃), halo(C₁-C₆)alkoxy (e.g., —OCF₃), C₁-C₆ alkylthio, arylthio, amino, amino(C₁-C₆)alkyl, nitro, 0-carbamyl, N-carbamyl, O-thiocarbamyl, N-thiocarbamyl, C-amido, N-amido, S-sulfonamido, N-sulfonamido, C-carboxy, O-carboxy, acyl, cyanato, isocyanato, thiocyanato, isothiocyanato, sulfinyl, sulfonyl, and oxo (═O).

Wherever a group is described as “optionally substituted” that group can be substituted with the above substituents.

The term R or the term R′, appearing by itself and without a number designation, unless otherwise defined, refers to a moiety chosen from hydrogen, alkyl, cycloalkyl, heteroalkyl, aryl, heteroaryl and heterocycloalkyl, any of which may be optionally substituted. Such R and R′ groups should be understood to be optionally substituted as defined herein. Whether an R group has a number designation or not, every R group, including R, R′ and R″ where n=(1, 2, 3, . . . n), every substituent, and every term should be understood to be independent of every other in terms of selection from a group. Should any variable, substituent, or term (e.g. aryl, heterocycle, R, etc.) occur more than one time in a formula or generic structure, its definition at each occurrence is independent of the definition at every other occurrence. Those of skill in the art will further recognize that certain groups may be attached to a parent molecule or may occupy a position in a chain of elements from either end as written. For example, an unsymmetrical group such as —C(O)N(R)— may be attached to the parent moiety at either the carbon or the nitrogen.

Asymmetric centers exist in the compounds or molecules disclosed herein. These centers are designated by the symbols “R” or “S,” depending on the configuration of substituents around the chiral carbon atom. It should be understood that the disclosure encompasses all stereochemical isomeric forms, including diastereomeric, enantiomeric, and epimeric forms, as well as d-isomers and 1-isomers, and mixtures thereof. Individual stereoisomers of compounds or molecules can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds or molecules of particular stereochemistry are either commercially available or can be made and resolved by techniques known in the art. Additionally, the compounds or molecules disclosed herein may exist as geometric isomers. The present disclosure includes all cis, trans, syn, anti, entgegen (E), and zusammen (Z) isomers as well as the appropriate mixtures thereof. Additionally, compounds or molecules may exist as tautomers; all tautomeric isomers are provided by this disclosure. Additionally, the compounds or molecules disclosed herein can exist in unsolvated as well as solvated forms with pharmaceutically acceptable solvents such as water, ethanol, and the like. In general, the solvated forms are considered equivalent to the unsolvated forms.

The term “bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position.

The term “disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

The phrase “therapeutically effective” is intended to qualify the amount of active ingredients used in the treatment of a disease or disorder or on the effecting of a clinical endpoint.

The term “therapeutically acceptable” refers to those compounds or molecules (or salts, prodrugs, tautomers, zwitterionic forms, etc.) which are suitable for use in contact with the tissues of patients without undue toxicity, irritation, and allergic response, are commensurate with a reasonable benefit/risk ratio, and are effective for their intended use.

As used herein, reference to “treatment” of a patient is intended to include prophylaxis. Treatment may also be preemptive in nature, i.e., it may include prevention of disease. Prevention of a disease may involve complete protection from disease, for example as in the case of prevention of infection with a pathogen, or may involve prevention of disease progression. For example, prevention of a disease may not mean complete foreclosure of any effect related to the diseases at any level, but instead may mean prevention of the symptoms of a disease to a clinically significant or detectable level. Prevention of diseases may also mean prevention of progression of a disease to a later stage of the disease.

The term “patient” is generally synonymous with the term “subject” and includes all mammals including humans. Examples of patients include humans, livestock such as cows, goats, sheep, pigs, and rabbits, and companion animals such as dogs, cats, rabbits, and horses. Preferably, the patient is a human.

The term “prodrug” refers to a compound or molecule that is made more active in vivo. Certain compounds or molecules disclosed herein may also exist as prodrugs, as described in Hydrolysis in Drug and Prodrug Metabolism: Chemistry, Biochemistry, and Enzymology (Testa, Bernard and Mayer, Joachim M. Wiley-VHCA, Zurich, Switzerland 2003). Prodrugs of the compounds described herein are structurally modified forms of the compound that readily undergo chemical changes under physiological conditions to provide the compound. Additionally, prodrugs can be converted to the compound by chemical or biochemical methods in an ex vivo environment. For example, prodrugs can be slowly converted to a compound when placed in a transdermal patch reservoir with a suitable enzyme or chemical reagent. Prodrugs are often useful because, in some situations, they may be easier to administer than the compound, or parent drug. They may, for instance, be bioavailable by oral administration whereas the parent drug is not. The prodrug may also have improved solubility in pharmaceutical compositions over the parent drug. A wide variety of prodrug derivatives are known in the art, such as those that rely on hydrolytic cleavage or oxidative activation of the prodrug. An example, without limitation, of a prodrug would be a compound which is administered as an ester (the “prodrug”), but then is metabolically hydrolyzed to the carboxylic acid, the active entity. Additional examples include peptidyl derivatives of a compound.

The compounds or molecules disclosed herein can exist as therapeutically acceptable salts. The present disclosure includes compounds or molecules listed above in the form of salts, including acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound or molecule in question. Basic addition salts may also be formed and be pharmaceutically acceptable. For a more complete discussion of the preparation and selection of salts, refer to Pharmaceutical Salts: Properties, Selection, and Use (Stahl, P. Heinrich. Wiley-VCHA, Zurich, Switzerland, 2002).

Basic addition salts can be prepared during the final isolation and purification of the compounds or molecules by reacting a carboxy group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine. Other representative organic amines useful for the formation of base addition salts include ethylenediamine, ethanolamine, diethanolamine, piperidine, and piperazine.

Other carrier materials and modes of administration known in the pharmaceutical art may also be used. Pharmaceutical compositions of the disclosure may be prepared by any of the well-known techniques of pharmacy, such as effective formulation and administration procedures. Preferred unit dosage formulations are those containing an effective dose, as herein below recited, or an appropriate fraction thereof, of the active ingredient.

It should be understood that in addition to the ingredients particularly mentioned above, the formulations described above may include other agents conventional in the art having regard to the type of formulation in question, for example those suitable for oral administration may include flavoring agents.

The amount of active ingredient that may be combined with the carrier materials to produce a single dosage form will vary depending upon the host treated and the particular mode of administration.

The compounds or molecules can be administered in various modes, e.g. orally, topically, or by injection. The precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the indication or condition being treated. In addition, the route of administration may vary depending on the condition and its severity. The above considerations concerning effective formulations and administration procedures are well known in the art and are described in standard textbooks.

### Combinations and Combination Therapy

In certain instances, it may be appropriate to administer at least one of the compounds described herein (or a pharmaceutically acceptable salt thereof) in combination with another therapeutic agent. By way of example only, if one of the side effects experienced by a patient upon receiving one of the compounds herein is hypertension, then it may be appropriate to administer an anti-hypertensive agent in combination with the initial therapeutic agent. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit of experienced by a patient may be increased by administering one of the compounds described herein with another therapeutic agent (which also includes a therapeutic regimen) that also has therapeutic benefit. By way of example only, in a treatment for diabetes involving administration of one of the compounds described herein, increased therapeutic benefit may result by also providing the patient with another therapeutic agent for diabetes. In any case, regardless of the disease, disorder or condition being treated, the overall benefit experienced by the patient may simply be additive of the two therapeutic agents or the patient may experience a synergistic benefit.

Specific, non-limiting examples of possible combination therapies include use of certain compounds of the disclosure with an ACE inhibitor.

In any case, the multiple therapeutic agents (at least one of which is a compound disclosed herein) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may be any duration of time ranging from a few minutes to four weeks.

Thus, in another aspect, certain embodiments provide methods for treating fxn-mediated disorders in a human or animal subject in need of such treatment comprising administering to said subject an amount of a compound disclosed herein effective to reduce or prevent said disorder in the subject, in combination with at least one additional agent for the treatment of said disorder that is known in the art. In a related aspect, certain embodiments provide therapeutic compositions comprising at least one compound disclosed herein in combination with one or more additional agents for the treatment of fxn-mediated disorders.

Besides being useful for human treatment, certain compounds and formulations disclosed herein may also be useful for veterinary treatment of companion animals, exotic animals and farm animals, including mammals, rodents, and the like. More preferred animals include horses, dogs, and cats.

### Compound Synthesis

Compounds of the present disclosure can be prepared using methods illustrated in general synthetic schemes and experimental procedures detailed below. General synthetic schemes and experimental procedures are presented for purposes of illustration and are not intended to be limiting. Starting materials used to prepare compounds of the present disclosure are commercially available or can be prepared using routine methods known in the art.

### List of Abbreviation

Ac₂O=acetic anhydride; AcCl=acetyl chloride; AcOH=acetic acid; AIBN=azobisisobutyronitrile; aq. =aqueous; Bu₃SnH=tributyltin hydride; CD₃OD=deuterated methanol; CDCl₃=deuterated chloroform; CDI=1,1′-Carbonyldiimidazole; DBU=1,8-diazabicyclo[5.4.0]undec-7-ene; DCM=dichloromethane; DEAD=diethyl azodicarboxylate; DIBAL-H=di-iso-butyl aluminium hydride; DIEA=DIPEA=N,N-diisopropylethylamine; DMAP=4-dimethylaminopyridine; DMF=N,N-dimethylformamide; DMSO-d₆=deuterated dimethyl sulfoxide; DMSO=dimethyl sulfoxide; DPPA=diphenylphosphoryl azide; EDC·HCl=EDCI·HCl=1-ethyl-3-(3-dimethylaminopropyl)carbodiimide hydrochloride; Et₂O=diethyl ether; EtOAc=ethyl acetate; EtOH=ethanol; h=hour; HATU=2-(1H-7-azabenzotriazol-1-yl)-1,1,3,3-tetramethyl uronium hexafluorophosphate methanaminium; HMDS=hexamethyldisilazane; HOBT=1-hydroxybenzotriazole; i-PrOH=isopropanol; LAH=lithium aluminium hydride; LiHMDS=Lithium bis(trimethylsilyl)amide; MeCN=acetonitrile; MeOH=methanol; MP-carbonate resin=macroporous triethylammonium methylpolystyrene carbonate resin; MsCI=mesyl chloride; MTBE=methyl tertiary butyl ether; MW=microwave irradiation; n-BuLi=n-butyllithium; NaHMDS=Sodium bis(trimethylsilyl)amide; NaOMe=sodium methoxide; NaOtBu=sodium t-butoxide; NBS═N-bromosuccinimide; NCS═N-chlorosuccinimide; NMP=N-Methyl-2-pyrrolidone; Pd(Ph3)₄=tetrakis(triphenylphosphine)palladium(0); Pd₂(dba)₃=tris(dibenzylideneacetone)dipalladium(0); PdCl₂(PPh₃)₂=bis(triphenylphosphine)palladium(II) dichloride; PG=protecting group; prep-HPLC=preparative high-performance liquid chromatography; PyBop=(benzotriazol-1-yloxy)-tripyrrolidinophosphonium hexafluorophosphate; Pyr=pyridine; RT=room temperature; RuPhos=2-dicyclohexylphosphino-2′,6′-diisopropoxybiphenyl; sat. =saturated; ss=saturated solution; t-BuOH=tert-butanol; T³P=Propylphosphonic Anhydride; TBS=TBDMS=tert-butyldimethylsilyl; TBSC1=TBDMSCI=tert-butyldimethylchlorosilane; TEA=Et₃N=triethylamine; TFA=trifluoroacetic acid; TFAA=trifluoroacetic anhydride; THF=tetrahydrofuran; Tol=toluene; TsCI=tosyl chloride; XPhos=2-dicyclohexylphosphino-2′,4′,6′-triisopropylbiphenyl.

### General Synthetic Methods for Preparing Compounds

In general, polyamides of the present disclosure may be synthesized by solid supported synthetic methods, using compounds such as Boc-protected straight chain aliphatic and heteroaromatic amino acids, and alkylated derivatives thereof, which are cleaved from the support by aminolysis, deprotected (e.g., with sodium thiophenoxide), and purified by reverse-phase HPLC, as well known in the art. The identity and purity of the polyamides may be verified using any of a variety of analytical techniques available to one skilled in the art such as ¹H-NMR, analytical HPLC, or mass spectrometry.

The following scheme can be used to practice the present disclosure:

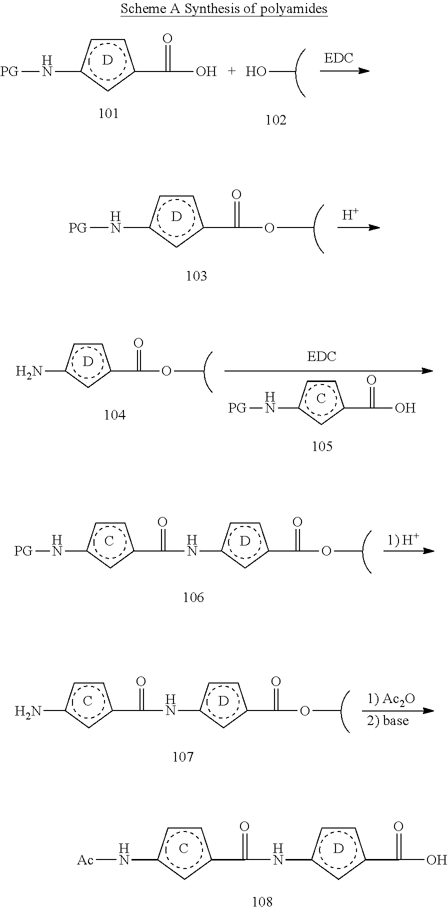

The compounds disclosed herein can be synthesized using Scheme I. For clarity and compactness, the scheme depicts the synthesis of a diamide comprising subunits “C” and “D”, both of which are represented as unspecified five-membered rings having amino and carboxy moieties. The amino group of subunit “D” is protected with a protecting group “PG” such as a Boc or CBz carbamate to give 101. The free )carboxylic acid is then reacted with a solid support, using a coupling reagent such as EDC, to give the supported compound 103. Removal of PG under acidic conditions gives the free amine 104, which is coupled with the nitrogen-protected carboxylic acid 105 to give amide 106. Removal of PG under acidic conditions gives the free amine 107. In this example, the free amine is reacted with acetic anhydride to form an acetamide (not shown. The molecule is then cleaved from the solid support under basic conditions to give carboxylic acid 108. Methods for attachment of the linker L and recruiting moiety X are disclosed below.

The person of skill will appreciate that many variations of the above scheme are available to provide a wide range of compounds:
1) The sequence 104-106-107 can be repeated as often as desired, in order to form longer polyamine sequences.
2) A variety of amino heterocycle carboxylic acids can be used, to form different subunits. Table 4, while not intended to be limiting, provides several heterocycle amino acids that are contemplated for the synthesis of the compounds in this disclosure. Carbamate protecting groups PG can be incorporated using techniques that are well established in the art.

TABLE 4
Heterocyclic amino acids.
Structure

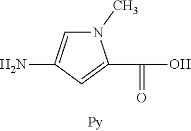

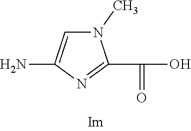

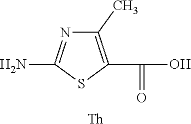

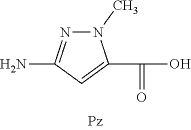

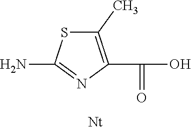

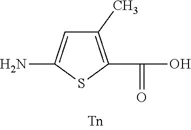

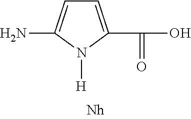

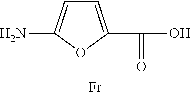

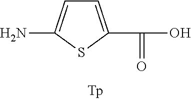

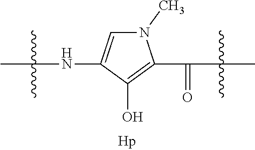 [SMILES: *Nc1cn(C)c(C(*)=O)c1O]

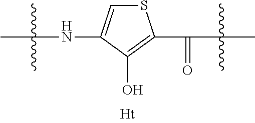 [SMILES: *Nc1csc(C(*)=O)c1O]

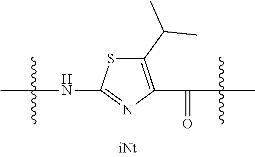 [SMILES: *Nc1nc(C(*)=O)c(C(C)C)s1]

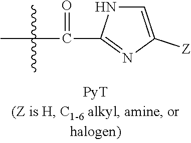

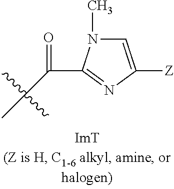

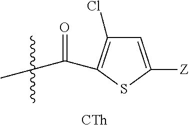

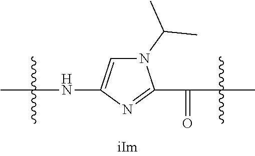 [SMILES: *Nc1cn(C(C)C)c(C(*)=O)n1]

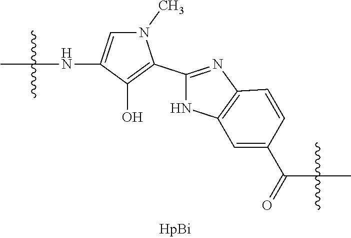 [SMILES: *Nc1cn(C)c(-c2nc3ccc(C(*)=O)cc3[nH]2)c1O]

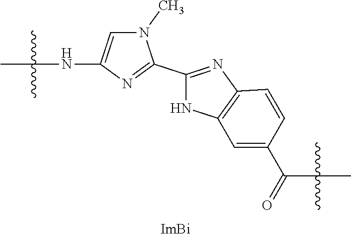 [SMILES: *Nc1cn(C)c(-c2nc3ccc(C(*)=O)cc3[nH]2)n1]

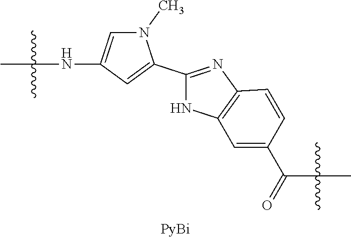 [SMILES: *Nc1cc(-c2nc3ccc(C(*)=O)cc3[nH]2)n(C)c1]

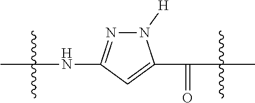 [SMILES: *Nc1cc(C(*)=O)[nH]n1]

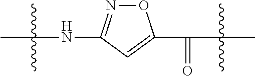 [SMILES: *Nc1cc(C(*)=O)on1]

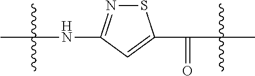 [SMILES: *Nc1cc(C(*)=O)sn1]

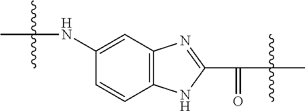 [SMILES: *Nc1ccc2[nH]c(C(*)=O)nc2c1]

3) Hydroxy-containing heterocyclic amino acids can be incorporated into Scheme I as their TBS ethers. While not intended to be limiting, Scheme B provides the synthesis of TBS-protected heterocyclic amino acids contemplated for the synthesis of the molecules in this disclosure.

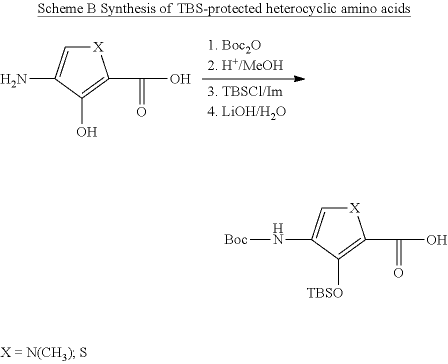

4) Aliphatic amino acids can be used in the above synthesis for the formation of spacer units “W” and subunits for recognition of DNA nucleotides. Table 5, while not intended to be limiting, provides several aliphatic amino acids contemplated for the synthesis of the or molecules in this disclosure.

TABLE 5
Aliphatic amino acids
Structure

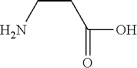

beta-alanine (□)

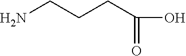

gamma-aminobutyric acid (“gAB” or □)

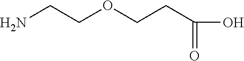

3-(2-aminoethoxy)propanoic acid

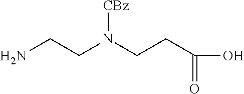

3-((2-aminoethyl)(2-oxo-2-phenyl-1β²-ethyl)
amino)propanoic acid

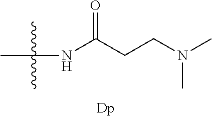 [SMILES: *NC(=O)CCN(C)C]

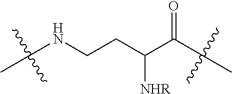

(R is H, C₁₋₆ alkyl)

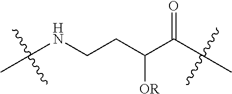

(R is H, C₁₋₆ alkyl, aryl, or heteroaryl)

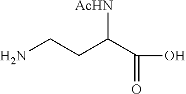 [SMILES: CC(=O)NC(CCN)C(=O)O]

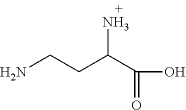

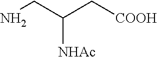 [SMILES: CC(=O)NC(CN)CC(=O)O]

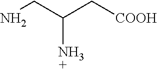

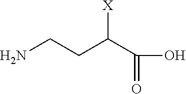

X is F or OH

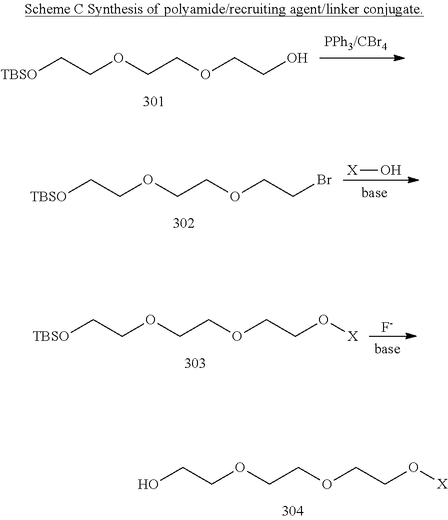

Attachment of the linker L and recruiting moiety X can be accomplished with the methods disclosed in Scheme C, which uses a triethylene glycol moiety for the linker L. The mono-TBS ether of triethylene glycol 301 is converted to the bromo compound 302 under Mitsunobu conditions. The recruiting moiety X is attached by displacement of the bromine with a hydroxyl moiety, affording ether 303. The TBS group is then removed by treatment with fluoride, to provide alcohol 304, which will be suitable for coupling with the polyamide moiety. Other methods will be apparent to the person of skill in the art for inclusion of alternate linkers L, including but not limited to propylene glycol or polyamine linkers, or alternate points of attachment of the recruiting moiety X, including but not limited to the use of amines and thiols.

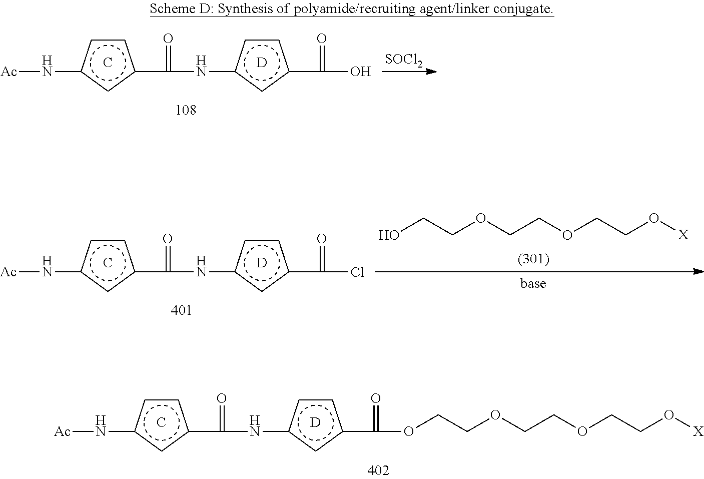

Synthesis of the X-L-Y molecule can be completed with the methods set forth in Scheme D. Carboxylic acid 108 is converted to the acid chloride 401. Reaction with the alcohol functionality of 301 under basic conditions provides the coupled product 402. Other methods will be apparent to the person of skill in the art for performing the coupling procedure, including but not limited to the use of carbodiimide reagents. For instance, the amide coupling reagents can be used, but not limited to, are carbodiimides such as dicyclohexylcarbodiimide (DCC), diisopropylcarbodiimide (DIC), ethyl-(N′,N′-dimethylamino)propylcarbodiimide hydrochloride (EDC), in combination with reagents such as 1-hydroxybenzotriazole (HOBt), 4-(N,N-dimethylamino)pyridine (DMAP) and diisopropylethylamine (DIEA). Other reagents are also often used depending the actual coupling reactions are (Benzotriazol-1-yloxy)tris(dimethylamino)phosphonium hexafluorophosphate (BOP), (Benzotriazol-1-yloxy)tripyrrolidinophosphonium hexafluorophosphate (PyBOP), (7-Azabenzotriazol-1-yloxy)tripyrrolidinophosphonium hexafluorophosphate (PyAOP), Bromotripyrrolidinophosphonium hexafluorophosphate (PyBrOP), Bis(2-oxo-3-oxazolidinyl)phosphinic chloride (BOP—Cl), O-(Benzotriazol-1-yl)-N,N,N′,N′-tetramethyluronium hexafluorophosphate (HBTU), O-(Benzotriazol-1-yl)-N,N,N′,N′-tetramethyluronium tetrafluoroborate (TBTU), O-(7-Azabenzotriazol-1-yl)-N,N,N′,N′-tetramethyluronium hexafluorophosphate (HATU), O-(7-Azabenzotriazol-1-yl)-N,N,N′,N′-tetramethyluronium tetrafluoroborate (TATU), O-(6-Chlorobenzotriazol-1-yl)-N,N,N′,N′-tetramethyluronium hexafluorophosphate (HCTU), Carbonyldiimidazole (CDI), and N,N,N′,N′-Tetramethylchloroformamidinium Hexafluorophosphate (TCFH).

### Attaching Protein Binding Molecules to Oligomeric Backbone

Generally the oligomeric backbone is functionalized to adapt to the type of chemical reactions can be performed to link the oligomers to the attaching position in protein binding moieties. The type reactions are suitable but not limited to, are amide coupling reactions, ether formation reactions (O-alkylation reactions), amine formation reactions (N-alkylation reactions), and sometimes carbon-carbon coupling reactions. The general reactions used to link oligomers and protein binders are shown in below schemes (E-G). The compounds and structures shown in Table 2 can be attached to the oligomeric backbone described herein at any position that is chemically feasible while not interfering with the hydrogen bond between the compound and the regulatory protein.

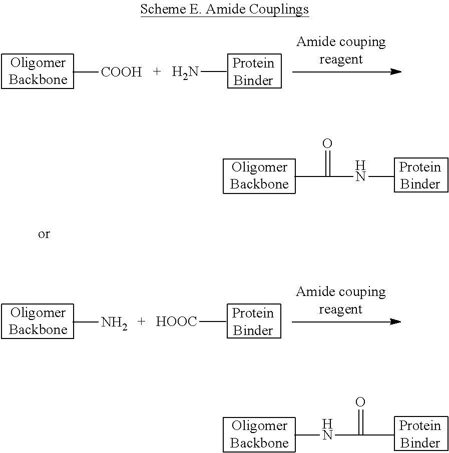

Either the oligomer or the protein binder can be functionalized to have a carboxylic acid and the other coupling counterpart being functionalized with an amino group so the moieties can be conjugated together mediated by amide coupling reagents. The amide coupling reagents can be used, but not limited to, are carbodiimides such as dicyclohexylcarbodiimide (DCC), diisopropylcarbodiimide (DIC), ethyl-(N′,N′-dimethylamino)propylcarbodiimide hydrochloride (EDC), in combination with reagents such as 1-hydroxybenzotriazole (HOBt), 4-(N,N-dimethylamino)pyridine (DMAP) and diisopropylethylamine (DIEA). Other reagents are also often used depending the actual coupling reactions are (Benzotriazol-1-yloxy)tris(dimethylamino)phosphonium hexafluorophosphate (BOP), (Benzotriazol-1-yloxy)tripyrrolidinophosphonium hexafluorophosphate (PyBOP), (7-Azabenzotriazol-1-yloxy)tripyrrolidinophosphonium hexafluorophosphate (PyAOP), Bromotripyrrolidinophosphonium hexafluorophosphate (PyBrOP), Bis(2-oxo-3-oxazolidinyl)phosphinic chloride (BOP—Cl), O-(Benzotriazol-1-yl)-N,N,N′,N′-tetramethyluronium hexafluorophosphate (HBTU), O-(Benzotriazol-1-yl)-N,N,N′,N′-tetramethyluronium tetrafluoroborate (TBTU), O-(7-Azabenzotriazol-1-yl)-N,N,N′,N′-tetramethyluronium hexafluorophosphate (HATU), O-(7-Azabenzotriazol-1-yl)-N,N,N′,N′-tetramethyluronium tetrafluoroborate (TATU), O-(6-Chlorobenzotriazol-1-yl)-N,N,N′,N′-tetramethyluronium hexafluorophosphate (HCTU), Carbonyldiimidazole (CDI), and N,N,N′,N′-Tetramethylchloroformamidinium Hexafluorophosphate (TCFH).

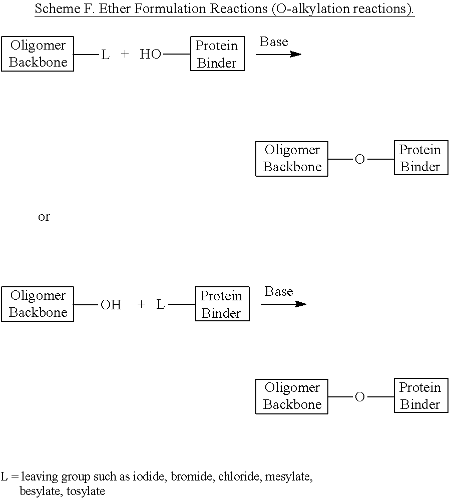

In an ether formation reaction, either the oligomer or the protein binder can be functionalized to have an hydroxyl group (phenol or alcohol) and the other coupling counterpart being functionalized with a leaving group such as halide, tosylate and mesylate so the moieties can be conjugated together mediated by a base or catalyst. The bases can be selected from, but not limited to, sodium hydride, potassium hydride, sodium hydroxide, potassium hydroxide, sodium carbonate, potassium carbonate. The catalyst can be selected from silver oxide, phase transfer reagents, iodide salts, and crown ethers.

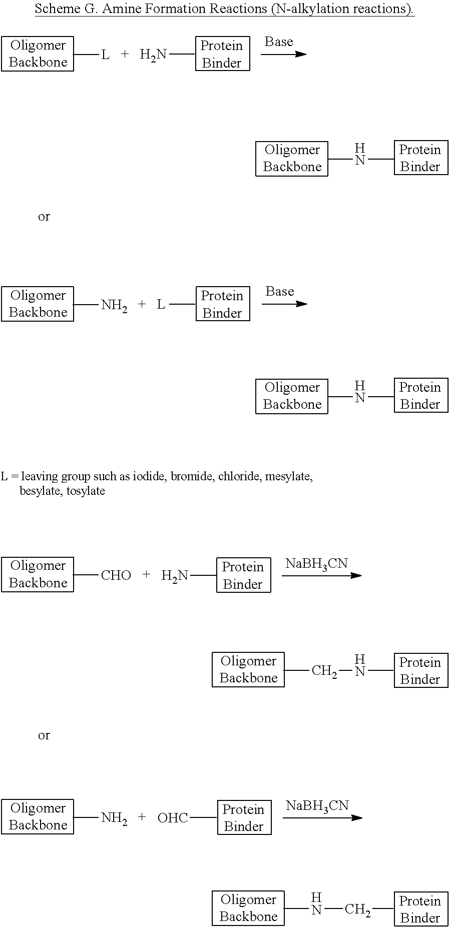

In an N-alkylation reaction, either the oligomer or the protein binder can be functionalized to have an amino group (arylamine or alkylamine) and the other coupling counterpart being functionalized with a leaving group such as halide, tosylate and mesylate so the moieties can be conjugated together directly or with a base or catalyst. The bases can be selected from, but not limited to, sodium hydride, potassium hydride, sodium hydroxide, potassium hydroxide, sodium carbonate, potassium carbonate. The catalyst can be selected from silver oxide, phase transfer reagents, iodide salts, and crown ethers. The alkylation of amines can also be achieved through reductive amination reactions, where in either the oligomer or the protein binder can be functionalized to have an amino group (arylamine or alkylamine) and the other coupling counterpart being functionalized with an aldehyde or ketone group so the moieties can be conjugated together with the treatment of a reducing reagent (hydride source) directly or in combination with a dehydration agent. The reducing reagents can be selected from, but not limited to, NaBH₄, NaHB(OAc)₃, NaBH₃CN, and dehydration agents are normally Ti(iPrO)₄, Ti(OEt)₄, Al(iPrO)₃, orthoformates and activated molecular sieves.

### Cell-Penetrating Ligand

In one aspect, the molecules of the present disclosure comprises a cell-penetrating ligand moiety. The cell-penetrating ligand moiety serves to facilitate transport of the compound across cell membranes. In certain embodiments, the cell-penetrating ligand moiety is a polypeptide. Several peptide sequences can facilitate passage into the cell, including polycationic sequences such as poly-R; arginine-rich sequences interspersed with spacers such as (RXR)ₙ (X=6-aminohexanoic acid) and (RXRRBR)ₙ (B=beta-alanine); sequences derived from the Penetratin peptide; and sequences derived from the PNA/PMO internalization peptide (Pip). The Pip5 series is characterized by the sequence ILFQY.

In certain embodiments, the cell-penetrating polypeptide comprises an N-terminal cationic sequence H₂N—(R)ₙ—CO—, with n=5-10, inclusive. In certain embodiments, the N-terminal cationic sequence contains 1, 2, or 3 substitutions of R for amino acid resides independently chosen from beta-alanine and 6-aminohexanoic acid.

In certain embodiments, the cell-penetrating polypeptide comprises the ILFQY sequence. In certain embodiments, the cell-penetrating polypeptide comprises the QFLY sequence. In certain embodiments, the cell-penetrating polypeptide comprises the QFL sequence.

In certain embodiments, the cell-penetrating polypeptide comprises a C-terminal cationic sequence —HN—(R)ₙ—COOH, with n=5-10, inclusive. In certain embodiments, the C-terminal cationic sequence contains 1, 2, or 3 substitutions of R for amino acid resides independently chosen from beta-alanine and 6-aminohexanoic acid. In certain embodiments, the C-terminal cationic sequence is substituted at every other position with an amino acid residue independently chosen from beta-alanine and 6-aminohexanoic acid. In certain embodiments, the C-terminal cationic sequence is —HN—RXRBRXRB-COOH.

TABLE 6
Cell-penetrating peptides.

| SEQ ID NO. | Sequence |
|---|---|
| SEQ ID NO. 1 | GRKKRRQRRRPPQ |
| SEQ ID NO. 2 | RQIKIWFQNRRMKWKK |
| SEQ ID NO. 3 | KLALKLALKALKAALKLA |
| SEQ ID NO. 4 | GWTLNS/AGYLLGKINLKALAALAKKIL |
| SEQ ID NO. 5 | NAKTRRHERRRKLAIER |
| SEQ ID NO. 6 | RRRRRRRR |
| SEQ ID NO. 7 | RRRRRRRRR |
| SEQ ID NO. 8 | GALFLGFLGAAGSTMGA |
| SEQ ID NO. 9 | KETWWETWWTEWSQPKKKRKV |
| SEQ ID NO. 10 | LLIILRRRIRKQAHAHSK |
| SEQ ID NO. 11 | YTAIAWVKAFIRKLRK |
| SEQ ID NO. 12 | IAWVKAFIRKLRKGPLG |
| SEQ ID NO. 13 | MVTVLFRRLRIRRACGPPRVRV |
| SEQ ID NO. 14 | GLWRALWRLLRSLWRLLWRA |
| SEQ ID NO. 15 | RRRRRRR QIKIWFQNRRMKWKKGG |
| SEQ ID NO. 16 | RXRRXRRXRIKILFQNRRMKWKK |
| SEQ ID NO. 17 | RXRRXRRXRIdKILFQNdRRMKWHKB |
| SEQ ID NO. 18 | RXRRXRRXRIHILFQNdRRMKWHKB |
| SEQ ID NO. 19 | RXRRBRRXRILFQYRXRBRXRB |
| SEQ ID NO. 20 | RXRRBRRXRILFQYRXRXRXRB |
| SEQ ID NO. 21 | RXRRXRILFQYRXRRXR |
| SEQ ID NO. 22 | RBRRXRRBRILFQYRBRXRBRB |
| SEQ ID NO. 23 | RBRRXRRBRILFQYRXRBRXRB |
| SEQ ID NO. 24 | RBRRXRRBRILFQYRXRRXRB |
| SEQ ID NO. 25 | RBRRXRRBRILFQYRXRBRXB |
| SEQ ID NO. 26 | RXRRBRRXRILFQYRXRRXRB |
| SEQ ID NO. 27 | RXRRBRRXRILFQYRXRBRXB |
| SEQ ID NO. 28 | RXRRBRRXRYQFLIRXRBRXRB |
| SEQ ID NO. 29 | RXRRBRRXRIQFLIRXRBRXRB |
| SEQ ID NO. 30 | RXRRBRRXRQFLIRXRBRXRB |
| SEQ ID NO. 31 | RXRRBRRXRQFLRXRBRXRB |
| SEQ ID NO. 32 | RXRRBRRXYRFLIRXRBRXRB |
| SEQ ID NO. 33 | RXRRBRRXRFQILYRXRBRXRB |
| SEQ ID NO. 34 | RXRRBRRXYRFRLIXRBRXRB |
| SEQ ID NO. 35 | RXRRBRRXILFRYRXRBRXRB |
| SEQ ID NO. 36 | Ac-RRLSYSRRRFXBpgG |
| SEQ ID NO. 37 | Ac-RRLSYSRRRFPFVYLIXBpgG |

Ac = acetyl; 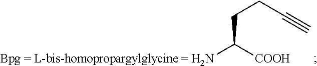
B = beta-alanine;
X = 6-aminohexanoic acid;
dK/dR = corresponding D-amino acid.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

While preferred embodiments of the present invention have been shown and described herein, it will be obvious to those skilled in the art that such embodiments are provided by way of example only.

Numerous variations, changes, and substitutions will now occur to those skilled in the art without departing from the invention. It should be understood that various alternatives to the embodiments described herein may be employed. It is intended that the following claims define the scope of the invention and that methods and structures within the scope of these claims and their equivalents be covered thereby.

### Synthesis of Representative Polyamides

### Example 1. Synthesis of 3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanoic acid (PA-001)

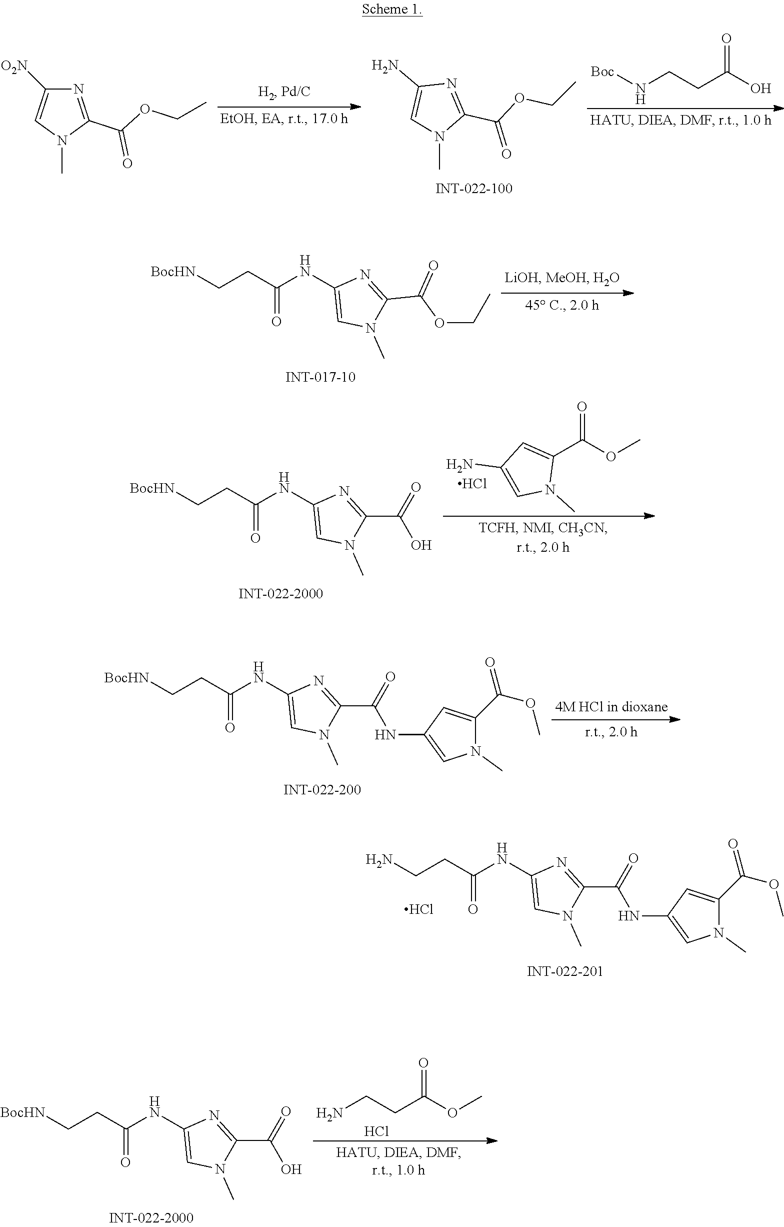

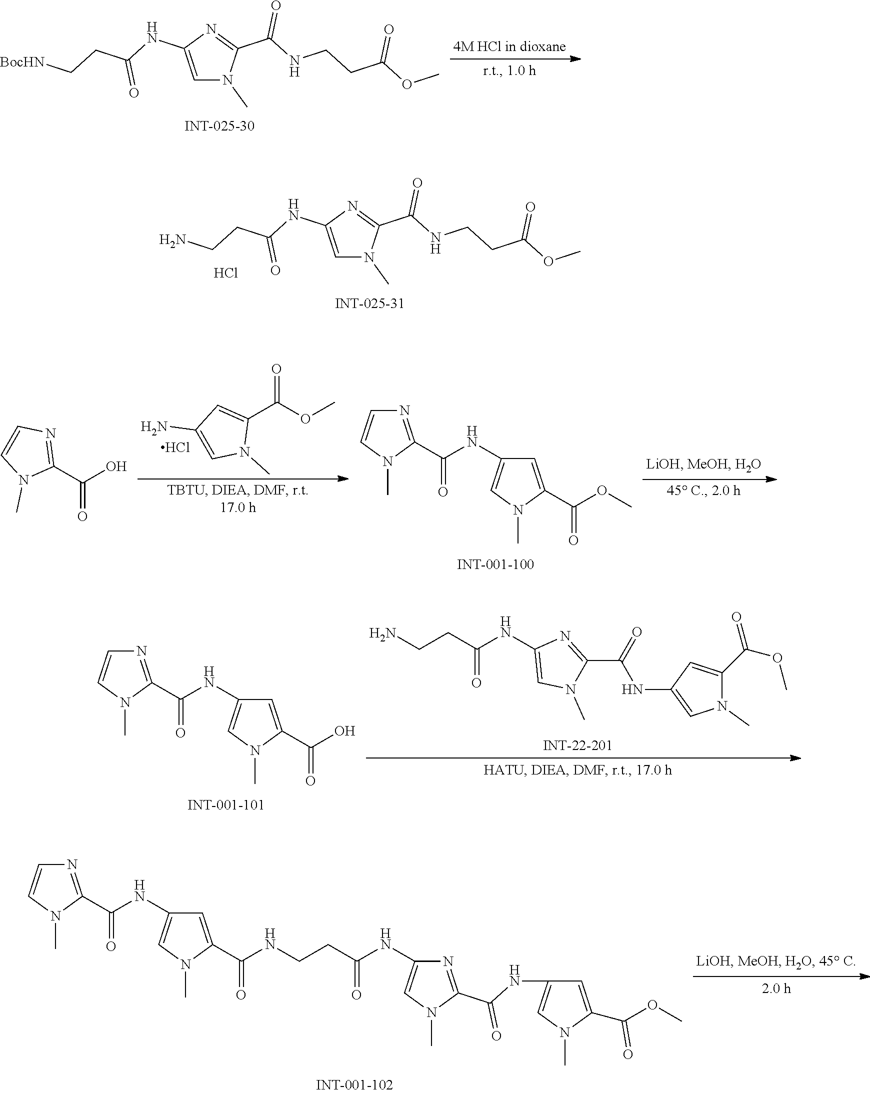

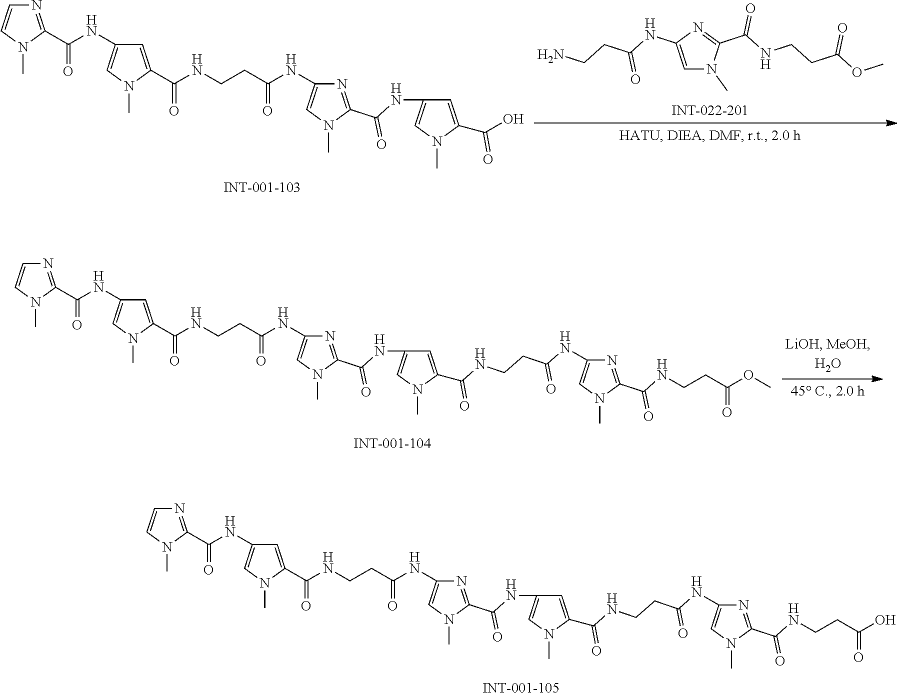 [SMILES: Cn1cc(NC(=O)c2nc(NC(=O)CCNC(=O)c3cc(NC(=O)c4nccn4C)cn3C)cn2C)cc1C(=O)O ; COC(=O)CCNC(=O)c1nc(NC(=O)CCN)cn1C ; COC(=O)CCNC(=O)c1nc(NC(=O)CCNC(=O)c2cc(NC(=O)c3nc(NC(=O)CCNC(=O)c4cc(NC(=O)c5nccn5C)cn4C)cn3C)cn2C)cn1C ; Cn1cc(NC(=O)c2nc(NC(=O)CCNC(=O)c3cc(NC(=O)c4nccn4C)cn3C)cn2C)cc1C(=O)NCCC(=O)Nc1cn(C)c(C(=O)NCCC(=O)O)n1]

Step 1: Synthesis of ethyl 4-amino-1-methylimidazole-2-carboxylate

To a solution of ethyl 1-methyl-4-nitroimidazole-2-carboxylate (30.00 g, 150.63 mmol, 1.00 equiv) in EtOH (120.00 mL) and EA (120.00 mL) was added Pd/C (8.01 g, 27% w/w). Then the reaction was stirred for 17.0 h at room temperature under H₂ atmosphere. The solid was filtrated out and the filtrate was concentrated to afford ethyl 4-amino-1-methylimidazole-2-carboxylate (22.30 g, 75.20%) as yellow solid. LC/MS: mass calcd. For C₇H₁₁N₃O₂: 169.09, found: 170.10 [M+H]⁺. ¹H NMR (400 MHz, DMSO-d6) δ: 7.37 (s, 1H), 4.29-4.34 (m, 2H), 3.94 (s, 3H), 1.31 (t, J=7.2 Hz, 3H).

Step 2: Synthesis of ethyl 4-[3-[(tert-butoxycarbonyl)amino]propanamido]-1-methylimidazole-2-carboxylate

Into a 500 mL flask was added 3-[(tert-butoxycarbonyl) amino]propanoic acid (22.45 g, 118.65 mmol, 0.90 equiv), DMF (180.00 mL). The mixture was cooled to 0 degrees C., then HATU (75.18 g, 197.71 mmol, 1.50 equiv) and DIEA (51.11 g, 395.43 mmol, 3.00 equiv) were added, the mixture was stirred for 10.0 mins, then ethyl 4-amino-1-methylimidazole-2-carboxylate (22.30 g, 131.81 mmol, 1.00 equiv) was added in portions. The reaction was stirred at room temperature for 1.0 h. The reaction was quenched with ice water (600 mL), and the solution was stirred for 15.0 min. The precipitated solids were collected by filtration and washed with water (3×50 mL) and dried under vacuum. This resulted in ethyl 4-[3-[(tert-butoxycarbonyl)amino] propanamido]-1-methylimidazole-2-carboxylate (34.50 g, 76.90%) as light yellow solid. LC/MS: mass calcd. For C₁₅H₂₄N₄O₅: 340.17, found: 341.20 [M+H]⁺. ¹H NMR (400 MHz, DMSO-d6) δ: 10.63 (s, 1H), 7.52 (s, 1H), 6.80 (t, J=5.6 Hz, 1H), 4.23-4.28 (m, 2H), 3.90 (s, 3H), 3.15-3.20 (m, 2H), 2.42 (t, J=7.2 Hz, 2H), 1.37 (s, 9H), 1.29 (t, J=7.2 Hz, 3H).

### Step 3: Synthesis of 4-[3-[(Tert-butoxycarbonyl)amino]propanamido]-1-methylimidazole-2-carboxylic Acid

To a stirred solution of ethyl 4-[3-[(tert-butoxycarbonyl)amino]propanamido]-1 Methylimidazole-2-carboxylate (34.50 g, 101.36 mmol, 1.00 equiv) in MeOH (200.00 mL) was added LiOH solution (2M, 202.00 mL, 4.00 equiv) dropwise at room temperature. The resulting mixture was stirred for 2.0 h at 45 degrees C. The resulting mixture was concentrated under reduced pressure. The residue was dissolved in H₂O (50 mL). The mixture was acidified to pH 3˜5 with 2M HCl. The precipitated solids were collected by filtration and washed with H₂O (3×30 mL), dried under vacuum. 4-[3-[(Tert-butoxycarbonyl)amino]propanamido]-1-methylimidazole-2-carboxylic acid (30.00 g, 94.77%) was obtained as white solid. LC/MS: mass calcd. For C₁₃H₂₀N₄O₅: 312.14, found: 313.15 [M+H]⁺. ¹H NMR (300 MHz, DMSO-d6) S: 10.53 (s, 1H), 7.48 (s, 1H), 6.79 (t, J=5.4 Hz, 1H), 3.89 (s, 3H), 3.15-3.22 (m, 2H), 2.43 (t, J=7.2 Hz, 2H), 1.37 (s, 9H).

### Step 4: Synthesis of Methyl 4-(4-[3-[(tert-butoxycarbonyl)amino]propanamido]-1-methylimidazole-2-amido)-1-methylpyrrole-2-carboxylate

To a stirred solution of 4-[3-[(tert-butoxycarbonyl)amino]propanamido]-1-methylimidazole-2-carboxylic acid (16.00 g, 51.23 mmol, 1.00 equiv) in CH₃CN (150.00 mL) was added TCFH (21.56 g, 76.84 mmol, 1.50 equiv), NMI (12.62 g, 153.69 mmol, 3.00 equiv) and methyl 4-amino-1-methylpyrrole-2-carboxylate hydrochloride (10.74 g, 56.34 mmol, 1.10 equiv) in portions at 0° C. The resulting mixture was stirred for 2.0 h at room temperature. The precipitated solids were collected by filtration and washed by CH₃CN (3×20 mL), dried under vacuum. Methyl 4-(4-[3-[(tert-butoxycarbonyl)amino]propanamido]-1-methylimidazole-2-amido)-1-methylpyrrole-2-carboxylate (19.00 g, 82.70%) was obtained as white solid. LC/MS: mass calcd. For C₂₀H₂₈N₆O₆: 448.21, found: 449.25 [M+H]⁺.

1H NMR (300 MHz, DMSO-d6) δ:10.24 (s, 1H), 10.11 (s, 1H), 7.52 (s, 1H), 7.33 (s, 1H), 6.99 (s, 1H), 6.82 (t, J=5.1 Hz, 1H), 3.94 (s, 3H), 3.85 (s, 3H), 3.74 (s, 3H), 3.16-3.23 (m, 2H), 2.47 (t, J=6.9 Hz, 2H), 1.38 (s, 9H).

### Step 5: Synthesis of Methyl 4-[4-(3-aminopropanamido)-1-methylimidazole-2-amido]-1-methylpyrrole-2-carboxylate hydrochloride

A solution of methyl 4-(4-[3-[(tert-butoxycarbonyl)amino]propanamido]-1-methylimidazole-2-amido)-1-methylpyrrole-2-carboxylate (19.00 g, 42.37 mmol, 1.00 equiv) in HCl/1,4-dioxane (4M, 200.00 mL) was stirred for 2.0 h at room temperature. The resulting mixture was concentrated under vacuum. Methyl 4-[4-(3-aminopropanamido)-1-methylimidazole-2-amido]-1-methylpyrrole-2-carboxylate hydrochloride (19.00 g crude) was obtained as yellow solid. LC/MS: mass calcd. For C15H21ClN6O 4: 348.15, found: 349.05 [M+H]⁺. 1H NMR (300 MHz, CD₃OD) δ: 7.37 (s, 2H), 6.91 (s, 1H), 4.03 (s, 3H), 3.88 (s, 3H), 3.79 (s, 3H), 3.09 (t, J=6.6 Hz, 2H), 2.64 (t, J=6.6 Hz, 2H).

### Step 6: Synthesis of methyl 3-[(4-[3-[(tert-butoxycarbonyl)amino]propanamido-1-methylimidazol-2-yl)formamido]propanoate

Into a 1000 ml flask was added 4-[3-[(tert-butoxycarbonyl)amino] propanamido]-1-methylimidazole-2-carboxylic acid (11.00 g, 35.22 mmol, 1.00 equiv), DMF (300.00 mL), the mixture was cooled to 0 degrees C., then HATU(20.09 g, 52.83 mmol, 1.50 equiv), DIEA (18.21 g, 140.88 mmol, 4.00 equiv) was added dropwise, the mixture was stirred for 10 mins, methyl 3-aminopropanoate (3.63 g, 35.22 mmol, 1.00 equiv) was added in portions. The reaction was stirred at room temperature for 1.0 h. The reaction mixture was poured into water/ice (600 mL), the solid was filtered out and dried under vacuum. The aqueous phase was extracted by EA (3×200 mL), the organic phases were combined and washed by H₂O (1×200 mL) and NaCl (1×200 mL), dried over anhydrous Na₂SO₄. After filtration, the filtrate was concentrated under reduced pressure. The residue was purified by silica gel column, eluted with pure EA. The fractions were combined and concentrated. Methyl 3-[(4-[3-[(tert-butoxycarbonyl)amino]propanamido]-1-methylimidazol-2-yl)formamido]propanoate (13.00 g, 87.95%) was obtained as yellow solid. LC/MS: mass calcd. For C₁₇H₂₇N₅O₆: 397.20, found: 398.20 [M+H]⁺. ¹H NMR (400 MHz, DMSO-d₆) δ: 10.28 (s, 1H), 7.92 (t, J=6.0 Hz, 1H), 7.37 (s, 1H), 6.77 (t, J=6.0 Hz, 1H), 3.88 (s, 3H), 3.59 (s, 3H), 3.42-3.47 (m, 2H), 3.13-3.18 (m, 2H), 2.56 (t, J=6.0 Hz, 2H), 2.42 (t, J=6.0 Hz, 2H), 1.35 (s, 9H).

### Step 7: Synthesis of methyl 3-[[4-(3-aminopropanamido)-1-methylimidazol-2-yl]formamido]propanoate hydrochloride

A solution of methyl 3-[(4-[3-[(tert-butoxycarbonyl)amino]propanamido]-1-methylimidazol-2-yl) formamido]propanoate (11.00 g, 27.678 mmol, 1.00 equiv) in HCl/1,4 dioxane (4M, 110.00 mL) was stirred for 1.0 h at room temperature. The resulting mixture was concentrated under vacuum to afford methyl 3-[[4-(3-aminopropanamido)-1-methylimidazol-2-yl]formamido]propanoate hydrochloride (11.00 g, crude) as yellow oil. LC/MS: mass calcd. For C₁₂H₁₉N₅O₄: 297.14, found: 298.20 [M+H]⁺. ¹H NMR (400 MHz, DMSO-d₆) δ: 10.57 (s, 1H), 7.92 (t, J=6.0 Hz, 1H), 7.37 (s, 1H), 3.89 (s, 3H), 3.59 (s, 3H), 3.43-3.47 (m, 2H), 2.97-3.05 (m, 2H), 2.57-2.71 (m, 2H), 2.56 (t, J=6.0 Hz, 2H).

### Step 8: Synthesis of Methyl 1-methyl-4-(1-methylimidazole-2-amido)pyrrole-2-carboxylate

To a stirred solution of 1-methylimidazole-2-carboxylic acid (10.00 g, 79.29 mmol, 7.00 equiv) in DMF (150.00 mL) was added TBTU (38.19 g, 118.94 mmol, 1.50 equiv), methyl 4-amino-1-methylpyrrole-2-carboxylate hydrochloride (16.63 g, 87.24 mmol, 1.10 equiv) and DIEA (30.74 g, 237.88 mmol, 3.00 equiv) in portions at 0 degrees C. The resulting mixture was stirred for 17.0 h at room temperature. The reaction was poured into water/Ice (450 mL). The precipitated solids were collected by filtration and washed with H₂O (3×50 mL), dried under vacuum. Methyl 1-methyl-4-(1-methylimidazole-2-amido)pyrrole-2-catboxylate (16.50 g, 78.37%) was obtained as white solid. LC/MS: mass calcd. For C₁₂H₁₄N₄O₃: 262.11, found: 263.15 [M+H]⁺. ¹H NMR (300 MHz, DMSO-d₆) δ:10.54 (s, 1H), 7.54 (s, 1H), 7.40 (s, 1H), 7.04 (s, 2H), 3.99 (s, 3H), 3.85 (s, 3H), 3.74 (s, 3H).

### Step 9: Synthesis of 1-Methyl-4-(1-methylimidazole-2-amido)pyrrole-2-carboxylic Acid

To a stirred solution of methyl 1-methyl-4-(1-methylimidazole-2-amido)pyrrole-2-carboxylate (16.50 g, 62.91 mmol, 1.00 equiv) in MeOH (100.00 mL) was added LiOH solution (2M, 158.00 mL, 5.00 equiv) dropwise at room temperature. The resulting mixture was stirred for 2.0 h at 45 degrees C. The resulting mixture was concentrated under reduced pressure. The residue was dissolved in H₂O (50 mL). The mixture was acidified to pH 3˜5 with 2M HCl. The precipitated solids were collected by filtration and washed with H₂O (3×30 mL), dried under vacuum. 1-Methyl-4-(1-methylimidazole-2-amido)pyrrole-2-carboxylic acid (12.00 g, 76.84%) was obtained as a white solid. LC/MS: mass calcd. For C₁₁H₁₂N₄O₃: 248.09, found: 249.10 [M+H]⁺. ¹H NMR (300 MHz, DMSO-d₆) δ: 10.52 (s, 1H), 7.48 (s, 1H), 7.41 (s, 1H), 7.06 (s, 1H), 6.99 (s, 1H), 3.99 (s, 3H), 3.82 (s, 3H).

### Step 10: Synthesis of methyl 1-methyl-4-[1-methyl-4-(3-[[1-methyl-4-(1-methylimidazole-2-amido)pyrrol-2-yl]formamido]propanamido)imidazole-2-amido]pyrrole-2-carboxylate

To a stirred solution of 1-methyl-4-(1-methylimidazole-2-amido)pyrrole-2-carboxylic acid (9.00 g, 36.255 mmol, 1.00 equiv) in DMF (150.00 mL) was added HATU (20.68 g, 54.38 mmol, 1.50 equiv), DIEA (14.06 g, 108.77 mmol, 3.00 equiv) and methyl 4-[4-(3-aminopropanamido)-1-methylimidazole-2-amido]-1-methylpyrrole-2-carboxylate (13.89 g, 39.872 mmol, 1.10 equiv) in portions at 0 degrees C. The resulting mixture was stirred for 17.0 h at room temperature. The reaction was poured into water/Ice (450 mL) at 0° C. The precipitated solids were collected by filtration and washed with H₂O(3×50 mL), dried under vacuum. Methyl 1-methyl-4-[1-methyl-4-(3-[[1-methyl-4-(1-methylimidazole-2-amido)pyrrol-2-yl]formamido]propanamido)imidazole-2-amido]pyrrole-2-carboxylate (14.00 g, 63.54%) was obtained as yellow solid. LC/MS: mass calcd. For C₂₆H₃₀N₁₀O₆: 578.23, found: 579.10 [M+H]⁺. 1H NMR (300 MHz, DMSO-d₆) δ: 10.53 (s, 1H), 10.29 (s, 1H), 10.11 (s, 1H), 8.10 (t, J=5.4 Hz, 1H), 7.52 (s, 1H), 7.47 (s, 2H), 7.25 (s, 1H), 7.17 (s, 1H), 6.99 (s, 1H), 6.97 (s, 1H), 3.99 (s, 3H), 3.95 (s, 3H), 3.84 (s, 3H), 3.82 (s, 3H), 3.69 (s, 3H), 3.42-3.49 (m, 2H), 2.60 (t, J=7.2 Hz, 2H).

### Step 11: Synthesis of 1-methyl-4-[1-methyl-4-(3-[[1-methyl-4-(1-methylimidazole-2-amido)pyrrol-2-yl]formamido]propanamido)imidazole-2-affordamido] pyrrole-2-carboxylic Acid

A solution of methyl 1-methyl-4-[1-methyl-4-(3-[[1-methyl-4-(1-methylimidazole-2-amido)pyrrol-2-yl]formamido]propanamido)imidazole-2-amido]pyrrole-2-yl]formamidocarboxylate (14.00 g, 24.20 mmol, 1.00 equiv) in MeOH (70.00 mL) was added LiOH (2M, 72.00 mL, 6.00 equiv). The mixture was stirred at 45 degrees C. for 2.0 h. The resulting mixture was concentrated under reduced pressure. The residue was dissolved in H₂O (50 mL). The mixture was acidified to pH 3˜5 with 2 M HCl. The precipitated solids were collected by filtration and washed with H₂O (3×20 mL), dried under vacuum. 1-methyl-4-[1-methyl-4-(3-[[1-methyl-4-(1-methylimidazole-2-amido)pyrrol-2-yl]formamido]propanamido)imidazole-2-affordamido] pyrrole-2-carboxylic acid (12.00 g, 81.49%) was obtained as yellow solid. LC/MS: mass calcd. For C₂₅H₂₈N₁₀O₆: 564.22, found: 565.15[M+H]⁺. ¹H NMR (300 MHz, DMSO-d6) δ:10.72 (s, 1H), 10.32 (s, 1H), 10.08 (s, 1H), 8.14 (t, J=6.0 Hz, 1H), 7.51 (s, 1H), 7.47 (s, 2H), 7.27 (s, 1H), 7.23 (s, 1H), 6.98 (s, 1H), 6.94 (s, 1H), 4.00 (s, 3H), 3.95 (s, 3H), 3.82 (s, 6H), 3.44-3.46 (m, 2H), 2.60 (t, J=6.6 Hz, 2H).

### Step 12: Synthesis of methyl 3-([1-methyl-4-[3-([1-methyl-4-[1-methyl-4-(3-[[1-methyl-4-(1-methylimidazole-2-amido)pyrrol-2-yl]formamido] propanamido)imidazole-2-amido]pyrrol-2-yl]formamido)propanamido]imidazol-2-yl]formamido)propanoate

To a stirred solution of 1-methyl-4-[1-methyl-4-(3-[[1-methyl-4-(1-methylimidazole-2-amido)pyrrol-2-yl]formamido]propanamido)imidazole-2-amido] pyrrole-2-carboxylic acid (12.00 g, 21.26 mmol, 1.00 equiv) in DMF (100.00 mL) was added HATU (12.12 g, 31.88 mmol, 1.50 equiv), DIEA (8.24 g, 63.77 mmol, 3.00 equiv) and methyl 3-[[4-(3-aminopropanamido)-1-methylimidazol-2-yl]formamido]propanoate (6.95 g, 23.38 mmol, 1.10 equiv) in portions at 0 degrees C. The resulting mixture was stirred for 2.0 h at room temperature. The reaction was poured into water/Ice (300 mL) at 0° C. The precipitated solids were collected by filtration and washed with H₂O(3×30 mL), dried under vacuum. Methyl 3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanoate (13.00 g, 64.77%) was obtained as yellow solid. LC/MS: mass calcd. For C₃₇H₄₅N₁₅O₉: 843.35, found: 844.55[M+H]+. 1H NMR (300 MHz, DMSO-d6) δ: 10.41 (s, 1H), 10.37 (s, 1H), 10.32 (s, 1H), 9.96 (s, 1H), 8.08 (s, 2H), 7.96 (s, 1H), 7.46 (s, 1H), 7.42 (s, 1H), 7.38 (s, 1H), 7.24 (s, 2H), 7.03 (s, 1H), 6.98 (s, 1H), 6.93 (s, 1H), 4.13 (s, 3H), 3.98 (s, 3H), 3.95 (s, 3H), 3.81 (s, 9H), 3.60 (s, 6H), 2.57-2.69 (m, 6H).

### Step 13: Synthesis of 3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanoic acid (PA-001)

A solution of methyl 3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanoate (10.00 g, 10.59 mmol, 1.00 equiv) in MeOH (60.00 mL) was added 2M LiOH (21.20 mL, 42.40 mmol, 4.00 equiv), the resulting mixture was stirred for 2.0 h at 45 degrees C. The resulting mixture was concentrated under reduced pressure. The resulting mixture was diluted with water (60 mL). The mixture was acidified to pH 3˜5 with 2M HCl. The precipitated solids were collected by filtration and washed with water (3×20 mL). The solid was dried under vacuum. This resulted in 3-([1-methyl-4-[3-([1-methyl-4-[1-methyl-4-(3-[[1-methyl-4-(1-methylimidazole-2-amido)pyrrol-2-yl]formamido]propanamido)imidazole-2-amido]pyrrol-2-yl]formamido)propanamido]imidazol-2-yl]formamido)propanoic acid (8.70 g, 84.14%) as a brown solid.

LC/MS: mass calcd. For C₃₆H₄₃N₁₅O₉: 829.34, found: 830.25[M+H]⁺. 1H NMR (300 MHz, DMSO-d₆) & 10.46 (s, 1H), 10.39 (s, 1H), 10.31 (s, 1H), 9.93 (s, 1H), 8.05-8.10 (m, 2H), 7.87 (t, J=6.0 Hz, 1H), 7.42-7.46 (m, 3H), 7.20-7.23 (m, 2H), 7.07 (s, 1H), 6.90-6.95 (m, 2H), 3.95 (s, 3H), 3.92 (s, 3H), 3.89 (s, 3H), 3.79 (s, 3H), 3.78 (s, 3H), 3.38-3.41 (m, 6H), 2.44-2.59 (m, 6H).

### Example 2. Synthesis of 1-Methyl-4-(1-methyl-4-{1-methyl-4-[1-methyl-4-(1-methylimidazole-2-amido)pyrrole-2-amido]pyrrole-2-aminoimidazole-2-amido)pyrrole-2-carboxylic acid (PA-047)

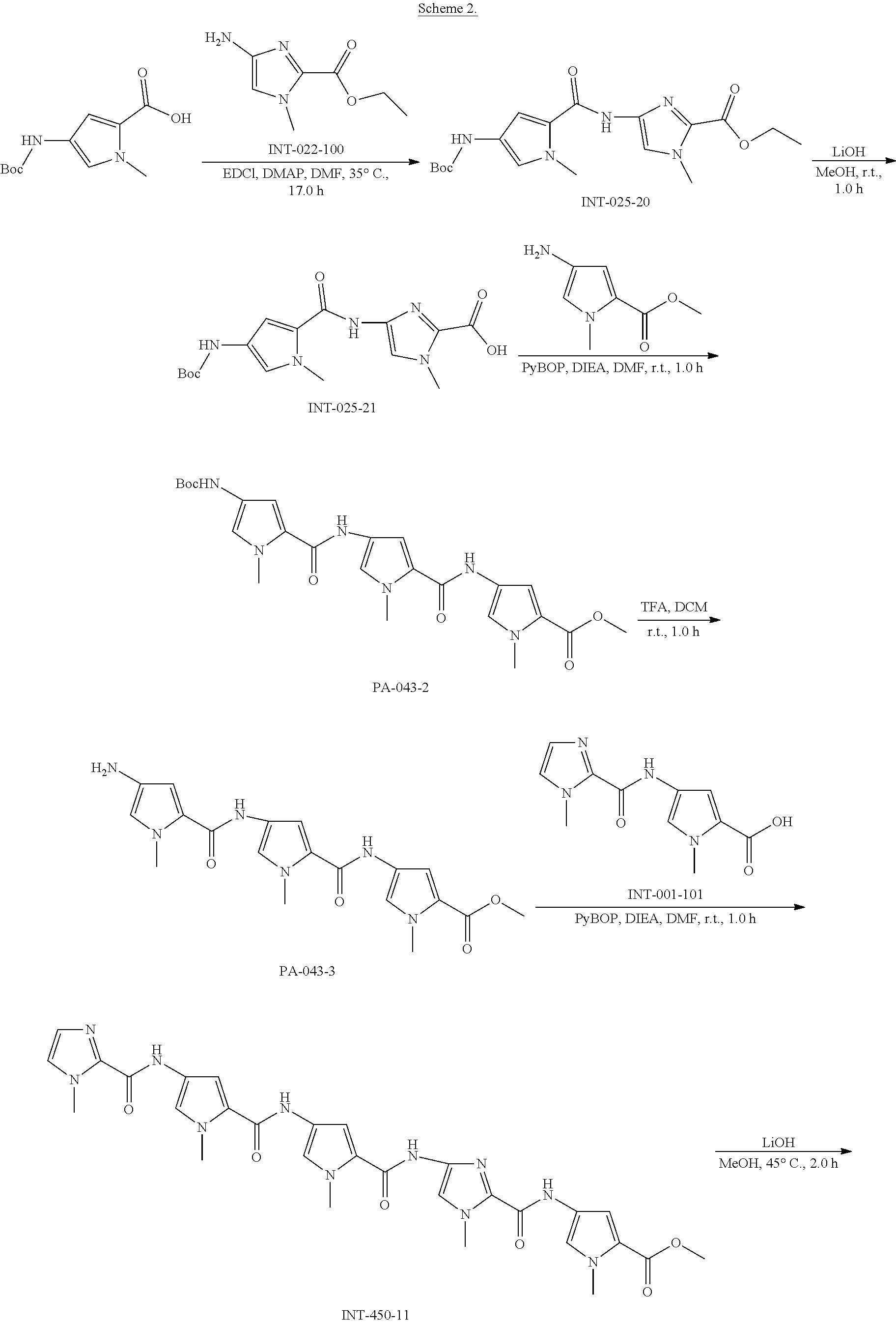

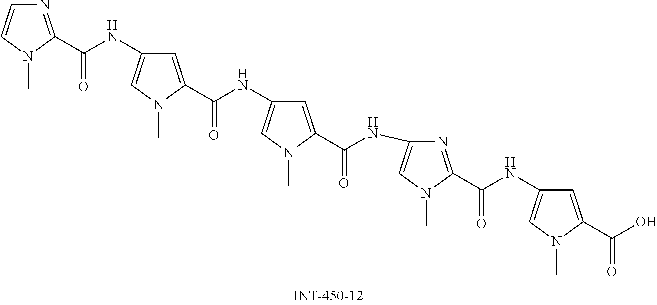 [SMILES: Cn1cc(NC(=O)c2nc(NC(=O)c3cc(NC(=O)c4cc(NC(=O)c5nccn5C)cn4C)cn3C)cn2C)cc1C(=O)O]

### Step 1: Synthesis of ethyl 4-[4-[(tert-butoxycarbonyl)amino)-1-methylpyrrole-2-amido)-1-methylimidazole-2-carboxylate

To a stirred solution of 4-[(tert-butoxycarbonyl)amino]-1-methylpyrrole-2-carboxylic acid (11.50 g, 47.87 mmol, 1.00 equiv) in DMF (200.00 mL) was added EDCI (22.94 g, 119.66 mmol, 2.50 equiv), ethyl 4-amino-1-methylimidazole-2-carboxylate (8.10 g, 47.87 mmol, 1.00 equiv) and DMAP (14.62 g, 119.66 mmol, 2.50 equiv) at 0 degrees C. The resulting mixture was stirred for 17.0 h at 35 degrees C. After reaction, the reaction was poured into 500 mL ice/water. The precipitated solids were collected by filtration and washed with water (3×50 mL), dried under vacuum. This resulted in ethyl 4-{4-[(tert-butoxycarbonyl)amino]-1-methylpyrrole-2-amido}-1-methylimidazole-2-carboxylate (16.00 g, 85.48% yield) as light yellow solid. LC/MS: mass calcd. For C₁₈H₂₅N₅O₅: 391.19, found: 392.30 [M+H]⁺.

### Step 2: Synthesis of 4-[4-[(tert-butoxycarbonyl)amino)-1-methylpyrrole-2-amido)-1-methylimidazole-2-carboxylic Acid

The procedure was the same as 4-[3-[(tert-butoxycarbonyl)amino] propanamido]-1-methylimidazole-2-carboxylic acid (Example 1 Step 1), but the reaction temperature was room temperature and the reaction time was 1.0 h. 970.00 mg of ethyl 4-[4-[(tert-butoxycarbonyl)amino]-1-methylpyrrole-2-amido]-1-methylimidazole-2-carboxylate was used, 638.00 mg of 4-[4-[(tert-butoxycarbonyl) amino]-1-methylpyrrole-2-amido]-1-methylimidazole-2-carboxylic acid was obtained as yellow solid (64.36% yield).

LC/MS: mass calcd. For C₁₆H₂₁N₅O₅: 363.15, found: 364.15 [M+H]⁺.

### Step 3: Synthesis of methyl 4-(4-[4-[(tert-butoxycarbonyl)amino]-1-methylpyrrole-2-amido}-1-methylimidazole-2-amido)-1-methylpyrrole-2-carboxylate

4-{4-[(tert-butoxycarbonyl)amino]-1-methylpyrrole-2-amido}-1-methylimidazole-2-carboxylic acid (6.00 g, 16.51 mmol, 1.00 equiv) was dissolved in DMF (60.00 mL). PyBOP (8.59 g, 16.51 mmol, 1.00 equiv), methyl 4-amino-1-methylpyrrole-2-carboxylate (2.55 g, 16.51 mmol, 1.00 equiv) and DIEA (6.40 g, 49.536 mmol, 3.00 equiv) were added in turn to the solution at 0 degrees C. The mixture was allowed to warm to room temperature and stirred for 1.0 h. After the reaction was completed, the mixture was added to the ice water (150 mL) dropwise. The solid was generated, filtered out, washed by water (2×15 mL) and dried under vacuum to afford methyl 4-(4-{4-[(tert-butoxycarbonyl)amino]-1-methylpyrrole-2-amido}-1-methylimidazole-2-amido)-1-methylpyrrole-2-carboxylate (7.10 g, 86.08%) as reddish brown solid. LC/MS: mass calcd. for C₂₃H₂₉N₇O₆: 499.21, found: 500.15 [M+H]⁺.

### Step 4: Synthesis of methyl 4-[4-(4-amino-1-methylpyrrole-2-amido)-1-methylimidazole-2-amido]-1-methylpyrrole-2-carboxylate

To a stirred solution of methyl 4-(4-{4-[(tert-butoxycarbonyl)amino]-1-methylpyrrole-2-amido}-1-methylimidazole-2-amido)-1-methylpyrrole-2-carboxylate (250.00 mg, 0.500 mmol, 1.00 equiv) in DCM (2.50 mL) was added TFA (0.50 mL) dropwise at room temperature. The resulting mixture was stirred for 1.0 h at room temperature. The resulting mixture was concentrated under vacuum. Methyl 4-[4-(4-amino-1-methylpyrrole-2-amido)-1-methylimidazole-2-amido]-1-methylpyrrole-2-carboxylate (250.00 mg, crude) was obtained as brown-yellow oil. LC/MS: mass calcd. For C₁₈H₂₁N₇O₄: 399.17, found: 400.35 [M+H]⁺.

### Step 5: Synthesis of methyl 1-methyl-4-(1-methyl-4-[1-methyl-4-[1-methyl-4-(1-methylimidazole-2-amido)pyrrole-2-amido)pyrrole-2-amido}imidazole-2-amido)pyrrole-2-carboxylate

To a stirred solution of 1-methyl-4-(1-methylimidazole-2-amido)pyrrole-2-carboxylic acid (156.62 mg, 0.63 mmol, 0.90 equiv) in DMF (2.00 mL) was added PyBOP (361.16 mg, 0.69 mmol, 1.00 equiv), methyl 4-[4-(4-amino-1-methylpyrrole-2-amido)-1-methylimidazole-2-amido]-1-methylpyrrole-2-carboxylate (280.00 mg, 0.70 mmol, 1.00 equiv) and DIEA (453.02 mg, 3.51 mmol, 5.00 equiv) in portions at 0 degrees C. The resulting mixture was stirred for 1.0 h at room temperature. The reaction mixture was purified by reverse phase column directly with the following conditions: column, C18 column; mobile phase, ACN in water (0.05% TFA), 5% to 70% gradient in 50 min; detector, UV 254 n. The fractions were combined and concentrated. Methyl 1-methyl-4-(1-methyl-4-{1-methyl-4-[1-methyl-4-(1-methylimidazole-2-amido)pyrrole-2-amido]pyrrole-2-amido}imidazole-2-amido)pyrrole-2-carboxylate (240.00 mg, 51.65% yield) was obtained as white solid. LC/MS: mass calcd. For C₂₉H₃₁N₁₁O₆: 629.25, found: 630.25 [M+H]⁺.

### Step 6: Synthesis of 1-Methyl-4-(1-methyl-4-[1-methyl-4-[1-methyl-4-(1-methylimidazole-2-amido)pyrrole-2-amido)pyrrole-2-amido}imidazole-2-amido)pyrrole-2-carboxylic acid (PA-047)

The procedure was the same as 4-[3-[(tert-butoxycarbonyl)amino] propanamido]-1-methylimidazole-2-carboxylic acid (Example 1 Step 3). 240.00 mg of methyl 1-methyl-4-(1-methyl-4-{1-methyl-4-[1-methyl-4-(1-methylimidazole-2-amido)pyrrole-2-amido]pyrrole-2-amido}imidazole-2-amido)pyrrole-2-carboxylate was used, 178.00 mg of 1-methyl-4-(1-methyl-4-{1-methyl-4-[1-methyl-4-(1-methylimidazole-2-amido)pyrrole-2-amido]pyrrole-2-amido}imidazole-2-amido)pyrrole-2-carboxylic acid was obtained as white solid (62.96% yield). LC/MS: mass calcd. For C₂₈H₂₉N₁₁O₆: 615.23, found: 616.25 [M+H]⁺.

### Example 3. Synthesis of 1-methyl-4-(1-methyl-4-{1-methyl-4-[2-(1-methylimidazol-2-yl)-3H-1,3-benzodiazole-5-aminopyrrole-2-amido}imidazole-2-amido)pyrrole-2-carboxylic acid (PA-047-P2BNZ)

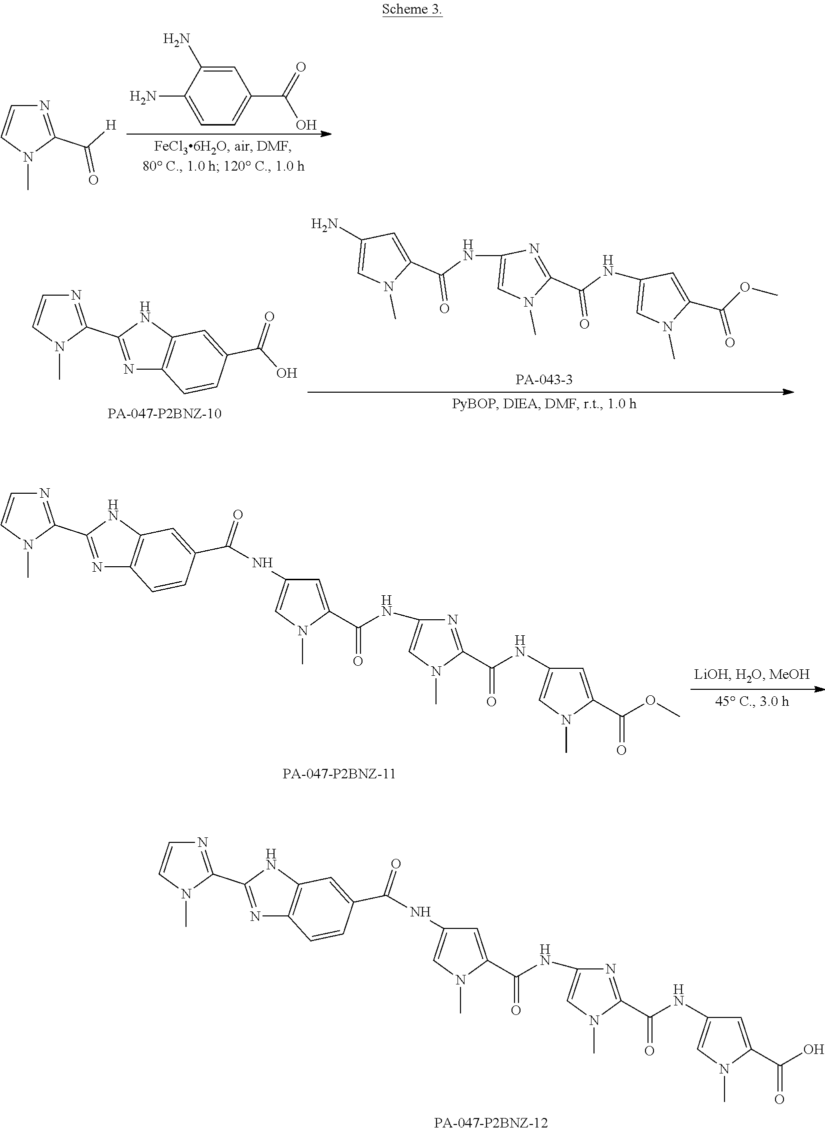

### Step 1: Synthesis of 2-(1-methylimidazol-2-yl)-3H-1,3-benzodiazole-5-carboxylic Acid

To a stirred solution of 3,4-diaminobenzoic acid (1.00 g, 6.57 mmol, 1.00 equiv) in DMF (15.00 mL) was added 1-methylimidazole-2-carbaldehyde (723.00 mg, 6.57 mmol, 1.00 equiv). The resulting mixture was stirred at 80 degrees C. for 1.0 h. FeCl₃·6H₂O (53.00 mg, 0.20 mmol, 0.03 equiv) was added in portions. The resulting mixture was stirred at 120 degrees C. for 1.0 h under air atmosphere. The resulting mixture was concentrated under vacuum. The residue was purified by silica gel column chromatography (0-10% EA/MeOH) to afford 2-(1-methylimidazol-2-yl)-3H-1,3-benzodiazole-5-carboxylic acid (130.00 mg, 8.17% yield) as yellow solid. LC/MS: mass calcd. For C₁₂H₁₀N₄O₂: 242.08, found: 243.10 [M+H]⁺.

### Step 2: Synthesis of methyl 1-methyl-4-(1-methyl-4-{1-methyl-4-[2-(1-methylimidazol-2-yl)-3H-1,3-benzodiazole-5-amido]pyrrole-2-amido}imidazole-2-amido)pyrrole-2-carboxylate

The procedure was the same as methyl 4-(4-{4-[(tert-butoxycarbonyl)amino]-1-methylpyrrole-2-amido}-1-methylimidazole-2-amido)-1-methylpyrrole-2-carboxylate (Example 2 Step 3). 120.00 mg of 2-(1-methylimidazol-2-yl)-3H-1,3-benzodiazole-5-carboxylic acid was used, 290.00 mg of methyl 1-methyl-4-(1-methyl-4-{1-methyl-4-[2-(1-methylimidazol-2-yl)-3H-1,3-benzodiazole-5-amido]pyrrole-2-amido}imidazole-2-amido)pyrrole-2-carboxylate was obtained as yellow solid (93.87% yield). LC/MS: mass calcd. For C₃₀H₂₉N₁₁O₅: 623.23, found: 624.50 [M+H]⁺.

### Step 3: Synthesis of 1-methyl-4-(1-methyl-4-{1-methyl-4-[2-(1-methylimidazol-2-yl)-3H-1,3-benzodiazole-5-amido]pyrrole-2-amido}imidazole-2-amido)pyrrole-2-carboxylic Acid

The procedure was the same as 4-[3-[(tert-butoxycarbonyl)amino] propanamido]-1-methylimidazole-2-carboxylic acid (Example 1 Step 3), but the reaction time was 3.0 h. 280.00 mg of methyl 1-methyl-4-(1-methyl-4-{1-methyl-4-[2-(1-methylimidazol-2-yl)-3H-1,3-benzodiazole-5-amido]pyrrole-2-amido}imidazole-2-amido)pyrrole-2-carboxylate was used, 210.00 mg of 1-methyl-4-(1-methyl-4-{1-methyl-4-[2-(1-methylimidazol-2-yl)-3H-1,3-benzodiazole-5-amido]pyrrole-2-amido}imidazole-2-amido)pyrrole-2-carboxylic acid was obtained as yellow solid (76.73% yield). LC/MS: mass calcd. For C₂₉H₂₇N₁₁O₅: 609.21, found: 610.45 [M+H]⁺.

### Example 4. Synthesis of 1-methyl-4-[3-({1-methyl-4-[1-methyl-4-(3-{[1-methyl-4-(1-methylimidazole-2-amido)pyrrol-2-yl]formamido]propanamido)imidazole-2-amido]pyrrol-2-yl]formamido)propanamido]imidazole-2-carboxylic Acid (PA-048)

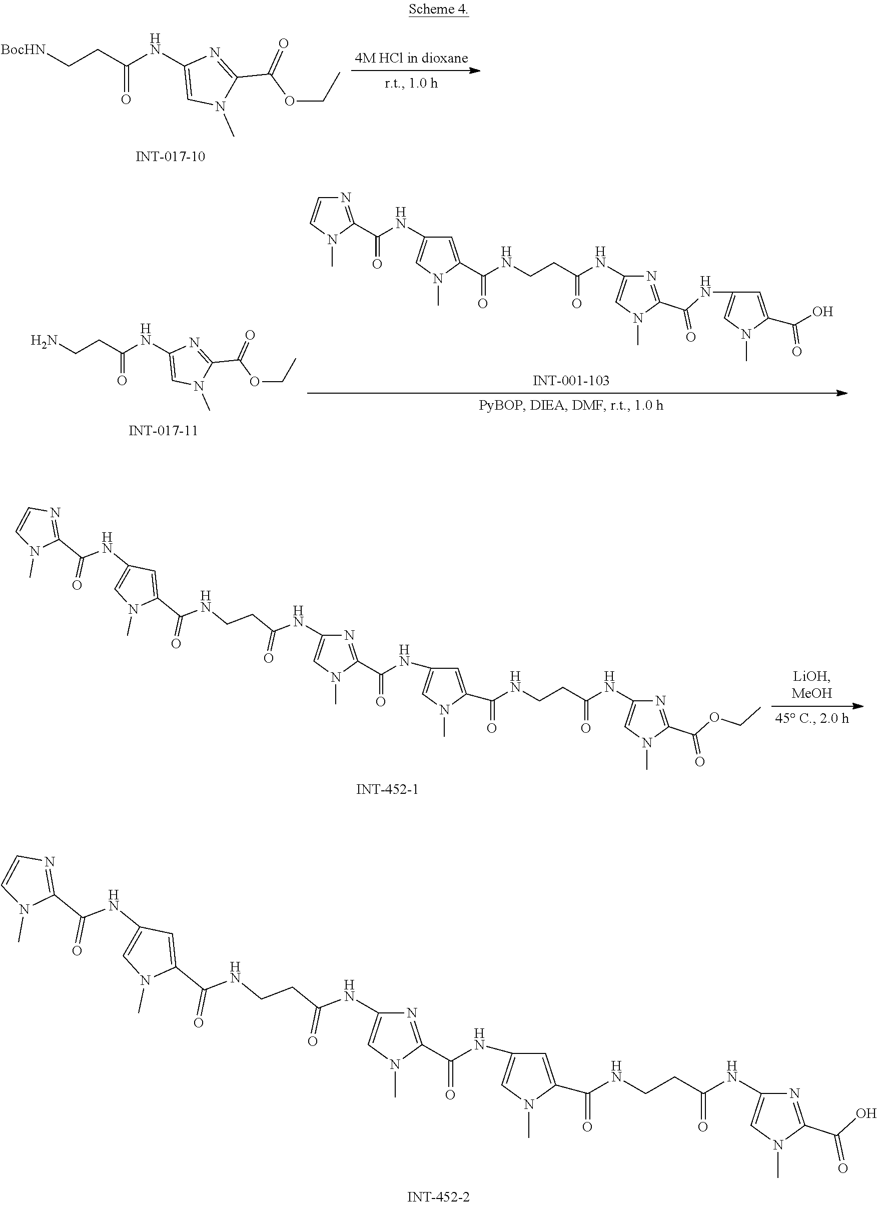 [SMILES: CCOC(=O)c1nc(NC(=O)CCNC(=O)OC(C)(C)C)cn1C ; CCOC(=O)c1nc(NC(=O)CCN)cn1C ; Cn1cc(NC(=O)c2nc(NC(=O)CCNC(=O)c3cc(NC(=O)c4nccn4C)cn3C)cn2C)cc1C(=O)O ; CCOC(=O)c1nc(NC(=O)CCNC(=O)c2cc(NC(=O)c3nc(NC(=O)CCNC(=O)c4cc(NC(=O)c5nccn5C)cn4C)cn3C)cn2C)cn1C ; Cn1cc(NC(=O)c2nc(NC(=O)CCNC(=O)c3cc(NC(=O)c4nccn4C)cn3C)cn2C)cc1C(=O)NCCC(=O)Nc1cn(C)c(C(=O)O)n1]

### Step 1: Synthesis of 2-(1-methylimidazol-2-yl)-3H-1,3-benzodiazole-5-carboxylic Acid

The procedure was the same as (Example 1 Step 7), but the reaction time was 1.0 h. 2.00 g of ethyl 4-[3-[(tert-butoxycarbonyl)amino]propanamido]-1-methylimidazole-2-carboxylate was used, 2.00 g crude of ethyl 4-(3-aminopropanamido)-1-methyl-1H-imidazole-2-carboxylate was obtained as off-white solid. LC/MS: mass calcd. For C₁₀H₁₆N₄O₃: 240.12, found: 241.10 [M+H]⁺.

### Step 2: Synthesis of ethyl 1-methyl-4-[3-({1-methyl-4-[1-methyl-4-(3-{[1-methyl-4-(1-methylimidazole-2-amido)pyrrol-2-yl]formamido}propanamido)imidazole-2-amido)pyrrol-2-yl}formamido)propanamido]imidazole-2-carboxylate

The procedure was the same as methyl 4-(4-{4-[(tert-butoxycarbonyl)amino]-1-methylpyrrole-2-amido}-1-methylimidazole-2-amido)-1-methylpyrrole-2-carboxylate (Example 2 Step 3). 270.00 mg of 1-methyl-4-[1-methyl-4-(3-{[1-methyl-4-(1-methylimidazole-2-amido)pyrrol-2-yl]formamido}propanamido)imidazole-2-amido]pyrrole-2-carboxylic acid was used, 460.00 mg of ethyl 1-methyl-4-[3-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)propanamido]imidazole-2-carboxylate was obtained as off-white solid (96.45% yield). LC/MS: mass calcd. For C₃₅H₄₂N₁₄O₈: 786.33, found: 809.60 [M+Na]+.

### Step 3: Synthesis of 1-methyl-4-[3-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)propanamido]imidazole-2-carboxylic Acid

The procedure was the same as 4-[3-[(Tert-butoxycarbonyl)amino] propanamido]-1-methylimidazole-2-carboxylic acid (Example 1 Step 3). 470.00 mg of ethyl 1-methyl-4-[3-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)propanamido]imidazole-2-carboxylate was used, 400.00 mg of 1-methyl-4-[3-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)propanamido]imidazole-2-carboxylic acid was obtained as off-white solid (74.41% yield). LC/MS: mass calcd. For C₃₃H₃₈N₁₄O₈:758.30, found: 759.55 [M+H]⁺.

### Example 5. Synthesis of 1-methyl-4-[2-(1-{1-methyl-4-[1-methyl-4-(3-{[1-methyl-4-(1-methylimidazole-2-amido)pyrrol-2-yl]formamido]propanamido)imidazole-2-amido]pyrrole-2-amido}cyclopropyl)acetamido]imidazole-2-carboxylic acid (PA-048-P6CP)

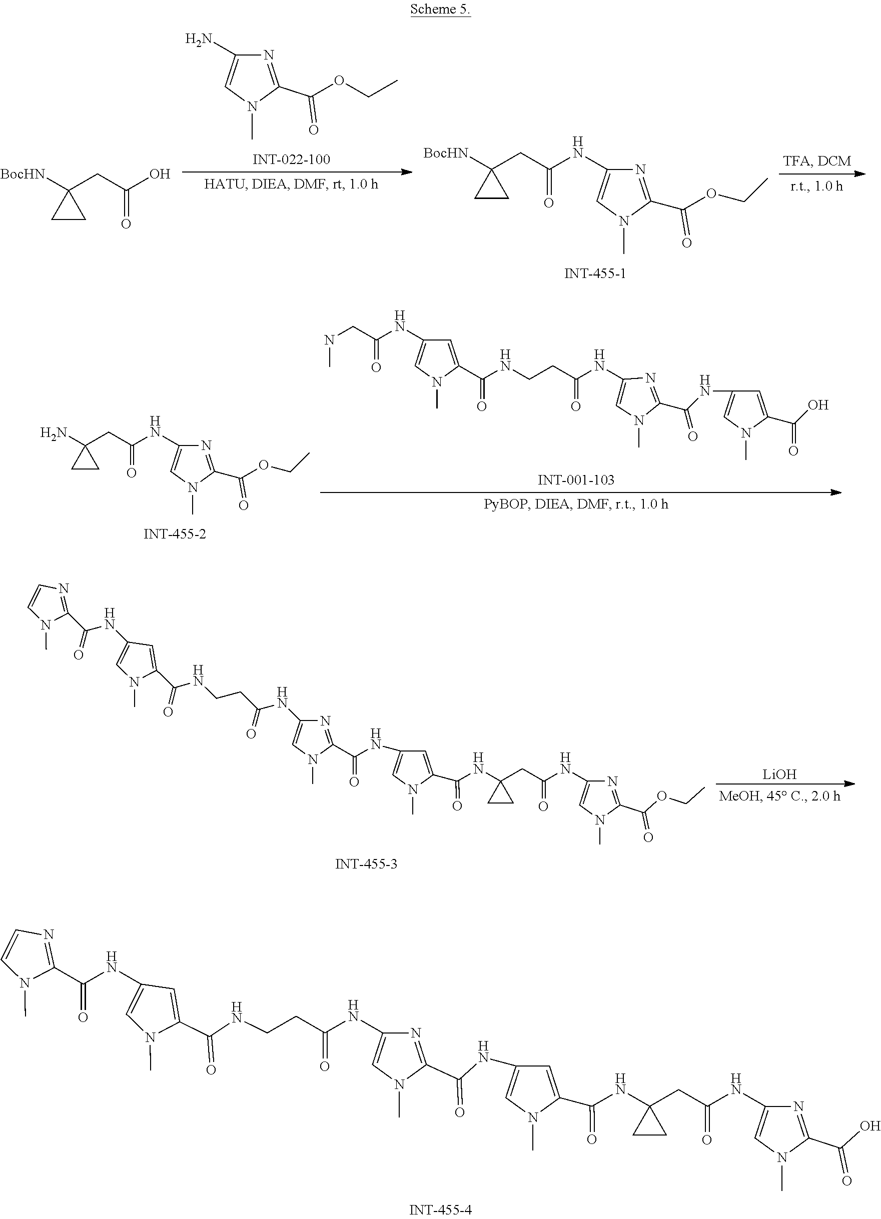

### Step 1: Synthesis of ethyl 4-(2-[1-[(tert-butoxycarbonyl)amino]cyclopropyl}acetamido)-1-methylimidazole-2-carboxylate

The procedure was the same as ethyl 3-[(4-[3-[(tert-butoxycarbonyl)amino] propanamido]-1-methylimidazol-2-yl)formamido]propanoate. 260.00 mg of {1-[(tert-butoxycarbonyl)amino]cyclopropyl}acetic acid was used, 320.00 mg of ethyl 4-(2-{1-[(tert-butoxycarbonyl)amino]cyclopropyl}acetamido)-1-methylimidazole-2-carboxylate was obtained as white solid (68.69% yield). LC/MS: mass calcd. For C₁₇H₂₆N₄O₅:366.19, found: 367.10 [M+H]⁺.

### Step 2: Synthesis of ethyl 4-[2-(1-aminocyclopropyl)acetamido]-1-methylimidazole-2-carboxylate

The procedure was the same as methyl 4-[4-(4-amino-1-methylpyrrole-2-amido)-1-methylimidazole-2-amido]-1-methylpyrrole-2-carboxylate (Example 2 Step 4). 260.00 mg of ethyl 4-(2-{1-[(tert-butoxycarbonyl)amino]cyclopropyl}acetamido)-1-methylimidazole-2-carboxylate was used, 260.00 mg crude of ethyl 4-[2-(1-aminocyclopropyl)acetamido]-1-methylimidazole-2-carboxylate was obtained as yellow oil. LC/MS: mass calcd. For C₁₂H₁₈N₄O₃: 266.14, found: 267.05[M+H]⁺.

### Step 3: Synthesis of ethyl 1-methyl-4-[2-(1-{1-methyl-4-[1-methyl-4-(3-{[1-methyl-4-(1-methylimidazole-2-amido)pyrrol-2-yl]formamido}propanamido)imidazole-2-amido]pyrrole-2-amido}cyclopropyl)acetamido]imidazole-2-carboxylate

The procedure was the same as methyl 4-(4-{4-[(tert-butoxycarbonyl)amino]-1-methylpyrrole-2-amido}-1-methylimidazole-2-amido)-1-methylpyrrole-2-carboxylate (Example 2 Step 3). 150.00 mg of ethyl 4-[2-(1-aminocyclopropyl)acetamido]-1-methylimidazole-2-carboxylate was used, 220.00 mg of ethyl 1-methyl-4-[2-(1-{1-methyl-4-[1-methyl-4-(3-{[1-methyl-4-(1-methylimidazole-2-amido)pyrrol-2-yl]formamido}propanamido)imidazole-2-amido]pyrrole-2-amido}cyclopropyl)acetamido]imidazole-2-carboxylate was obtained as yellow solid (37.48% yield). LC/MS: mass calcd. For C₃₇H₄₄N₁₄O₈: 812.35, found: 813.35 [M+H]⁺.

### Step 4: Synthesis of 1-methyl-4-[2-(1-{1-methyl-4-[1-methyl-4-(3-{[1-methyl-4-(1-methylimidazole-2-amido)pyrrol-2-yl]formamido}propanamido)imidazole-2-amido]pyrrole-2-amido}cyclopropyl)acetamido]imidazole-2-carboxylic Acid

The procedure was the same as 4-[3-[(tert-butoxycarbonyl)amino] propanamido]-1-methylimidazole-2-carboxylic acid (Example 1 Step 3). 240.00 mg of ethyl 1-methyl-4-[2-(1-{1-methyl-4-[1-methyl-4-(3-{[1-methyl-4-(1-methylimidazole-2-amido)pyrrol-2-yl]formamido}propanamido)imidazole-2-amido]pyrrole-2-amido}cyclopropyl)acetamido]imidazole-2-carboxylate was used, 155.00 mg of 1-methyl-4-[2-(1-{1-methyl-4-[1-methyl-4-(3-{[1-methyl-4-(1-methylimidazole-2-amido)pyrrol-2-yl]formamido}propanamido)imidazole-2-amido]pyrrole-2-amido}cyclopropyl)acetamido]imidazole-2-carboxylic acid was obtained as white solid (54.85% yield). LC/MS: mass calcd. For C₃₅H₄₀N₁₄O₈: 784.32, found: 785.55 [M+H]⁺.

### Example 6. Synthesis of 1-methyl-4-[3-methyl-3-({1-methyl-4-[1-methyl-4-(3-{[1-methyl-4-(1-methylimidazole-2-amido)pyrrol-2-yl]formamido]propanamido)imidazole-2-amido]pyrrol-2-yl}formamido)butanamido]imidazole-2-carboxylic acid (PA-048-P6GM)

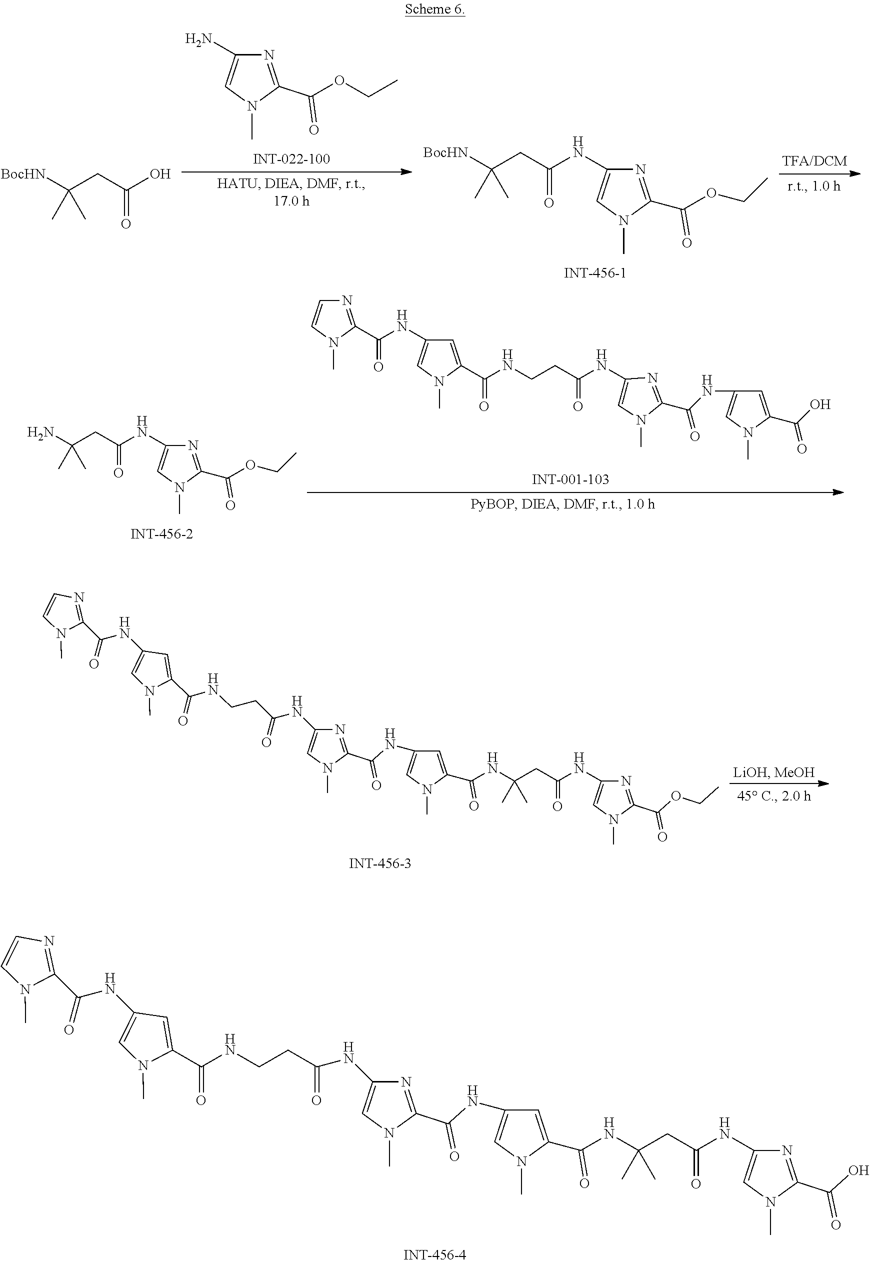

### Step 1: Synthesis of ethyl 4-[3-[(tert-butoxycarbonyl)amino)-3-methylbutanamido}-1-methylimidazole-2-carboxylate

The procedure was the same as methyl 3-[(4-[3-[(tert-butoxycarbonyl)amino] propanamido]-1-methylimidazol-2-yl)formamido]propanoate, but the reaction time was 17.0 h and the crude product without purification by silica gel column. 385.00 mg of 3-[(tert-butoxycarbonyl)amino]-3-methylbutanoic acid was used, 540.00 mg crude of ethyl 4-{3-[(tert-butoxycarbonyl)amino]-3-methylbutanamido}-1-methylimidazole-2-carboxylate was obtained as white solid. LC/MS: mass calcd. For C₁H₂₈N₄O₅: 368.21, found: 369.15 [M+H]⁺.

### Step 2: Synthesis of ethyl 4-(3-amino-3-methylbutanamido)-1-methylimidazole-2-carboxylate

The procedure was the same as methyl 4-[4-(4-amino-1-methylpyrrole-2-amido)-1-methylimidazole-2-amido]-1-methylpyrrole-2-carboxylate (Example 2 Step 3). 540.00 mg of ethyl 4-{3-[(tert-butoxycarbonyl)amino]-3-methylbutanamido}-1-methylimidazole-2-carboxylate was used, 540.00 mg crude of ethyl 4-(3-amino-3-methylbutanamido)-1-methylimidazole-2-carboxylate was obtained as yellow oil. LC/MS: mass calcd. For C₁₂H₂₀N₄O₃: 268.15, found: 269.30 [M+H]⁺.

### Step 3: Synthesis of ethyl 1-methyl-4-[3-methyl-3-({1-methyl-4-[1-methyl-4-(3-{]1-methyl-4-(1-methylimidazole-2-amido)pyrrol-2-yl]formamido}propanamido)imidazole-2-amido]pyrrol-2-yl}formamido)butanamido)imidazole-2-carboxylate

To a stirred solution of 1-methyl-4-[1-methyl-4-(3-{[1-methyl-4-(1-methylimidazole-2-amido)pyrrol-2-yl]formamido}propanamido)imidazole-2-amido]pyrrole-2-carboxylic acid (600.00 mg, 1.06 mmol, 1.00 equiv) in DMF (5.00 mL) was added DIEA (412.07 mg, 3.19 mmol, 3.00 equiv), ethyl 4-(3-amino-3-methylbutanamido)-1-methylimidazole-2-carboxylate (313.67 mg, 1.17 mmol, 1.10 equiv) and PyBOP (1106.11 mg, 2.13 mmol, 2.00 equiv) in portions at 0 degrees C. The resulting mixture was stirred for 17.0 h at room temperature. The reaction was poured into ice/water (15 mL). The precipitated solids were collected by filtration and washed with H₂O (3×2 mL), dried under vacuum. The reaction mixture was purified by silica gel column chromatography, eluted with DCM/MeOH (5:1) to afford ethyl1-methyl-4-[3-methyl-3-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-carboxylate (800.00 mg, 77.49% yield) as yellow oil. LC/MS: mass calcd. For C₃₇H₄₆N₁₄O₈: 814.36, found: 408.50 [M/2/+H]+.

### Step 4: Synthesis of 1-methyl-4-[3-methyl-3-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-carboxylic Acid

To a stirred solution of ethyl 1-methyl-4-(3-methyl-3-{1-methyl-4-[1-methyl-4-(3-{[1-methyl-4-(1-methylimidazole-2-amido)pyrrol-2-yl]formamido}propanamido)imidazole-2-amido]pyrrole-2-carbonyloxy}butanamido)imidazole-2-carboxylate (725.00 mg, 0.89 mmol, 1.00 equiv) in MeOH (8.00 mL) was added LiOH solution (2M in H₂O, 1.80 mL, 4.00 equiv) at room temperature. The resulting mixture was stirred for 2.0 h at 45 degrees C. The resulting mixture was concentrated under reduced pressure. The residue was dissolved in H₂O (9 mL). The mixture was acidified to pH 3˜5 with 2M HCl. Then the mixture was concentrated and the residue was purified by reverse phase column directly with the following conditions: column, C18 silica gel; mobile phase, ACN in water (0.05% TFA), 10% to 50% gradient in 50 min; detector, UV 254 nm. The fractions were combined and concentrated. 1-Methyl-4-[3-methyl-3-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-carboxylic acid (700.00 mg, 95.88% yield) was obtained as yellow solid. LC/MS: mass calcd. For C₃₅H₄₂N₁₄O₈: 786.33, found: 394.40 [M/2+H]⁺.

### Example 7. Synthesis of 1-methyl-4-[(1r, 3r)-3-{1-methyl-4-[1-methyl-4-(3-{[1-methyl-4-(1-methylimidazole-2-amido)pyrrol-2-yl]formamido}propanamido)imidazole-2-amido]pyrrole-2-amido}cyclobutaneamido]imidazole-2-carboxylic Acid (PA-048-P6CB)

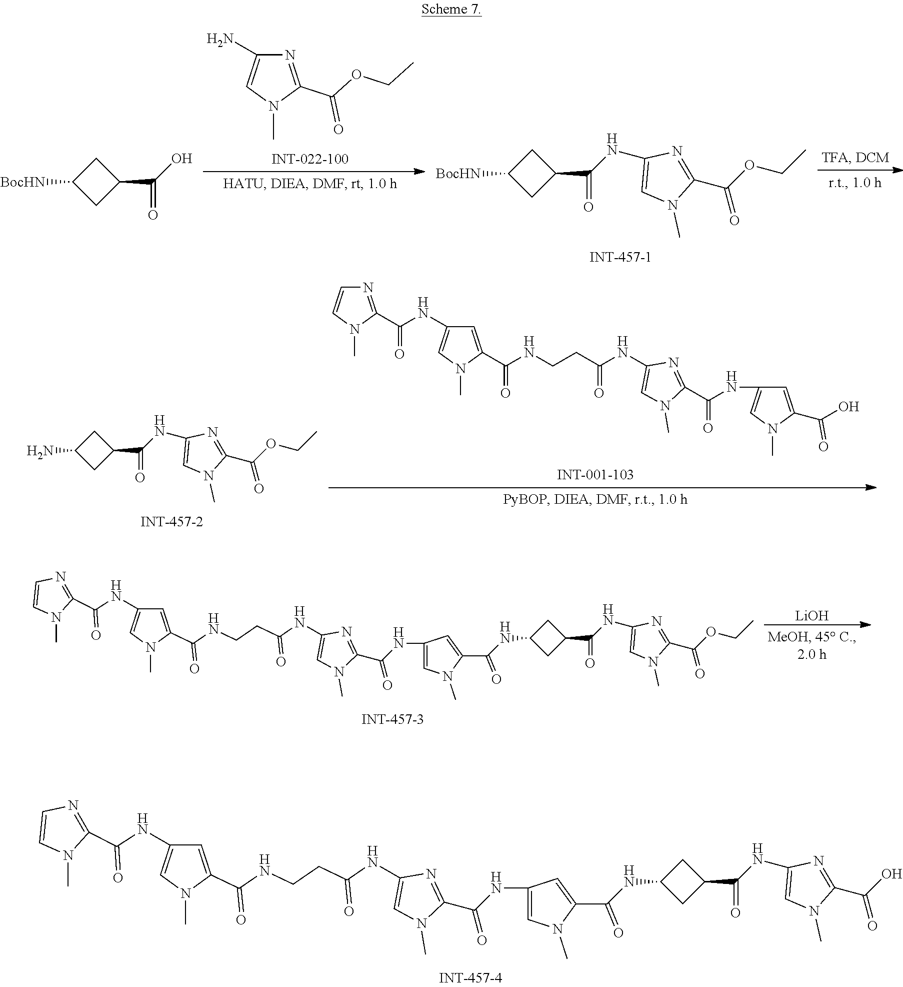

### Step 1: Synthesis of ethyl 1-methyl-4-[(1r, 3r)-3-[(tert-butoxycarbonyl)amino]cyclobutaneamido]imidazole-2-carboxylate

The procedure was the same as methyl 3-[(4-[3-[(tert-butoxycarbonyl)amino] propanamido]-1-methylimidazol-2-yl)formamido]propanoate. 200.00 mg of (1r, 3r)-3-[(tert-butoxycarbonyl)amino]cyclobutane-1-carboxylic acid was used, 330.00 mg of ethyl 1-methyl-4-[(1r, 3r)-3-[(tert-butoxycarbonyl)amino] cyclobutaneamido]imidazole-2-carboxylate was obtained as orange solid (96.93% yield). LC/MS: mass calcd. For C₁₇H₂₆N₄O₅: 366.19, found: 367.25 [M+H]⁺.

### Step 2: Synthesis of ethyl 1-methyl-4-[(1r, 3r)-3-aminocyclobutaneamido] imidazole-2-carboxylate

The procedure was the same as methyl 4-[4-(4-amino-1-methylpyrrole-2-amido)-1-methylimidazole-2-amido]-1-methylpyrrole-2-carboxylate (Example 2 Step 3). 145.00 mg of ethyl 1-methyl-4-[(1r, 3r)-3-[(tert-butoxycarbonyl)amino]cyclobutaneamido]imidazole-2-carboxylate was used, 145.00 mg crude of ethyl 1-methyl-4-[(1r, 3r)-3-aminocyclobutaneamido]imidazole-2-carboxylate was obtained as yellow oil. LC/MS: mass calcd. For C₁₂H₁i₈N₄O₃: 266.14, found: 267.10 [M+H]⁺.

### Step 3: Synthesis of ethyl 1-methyl-4-[(1r, 3r)-3-[1-methyl-4-[1-methyl-4-(3-{[1-methyl-4-(1-methylimidazole-2-amido)pyrrol-2-yl]formamido}propanamido)imidazole-2-amido]pyrrole-2-amido}cyclobutaneamido]imidazole-2-carboxylate

The procedure was the same as methyl 4-(4-{4-[(tert-butoxycarbonyl)amino]-1-methylpyrrole-2-amido}-1-methylimidazole-2-amido)-1-methylpyrrole-2-carboxylate (Example 2 Step 3). 105.00 mg of ethyl 1-methyl-4-[(1r, 3r)-3-aminocyclobutaneamido]imidazole-2-carboxylate was used, 250.00 mg of ethyl 1-methyl-4-[(1r, 3r)-3-{1-methyl-4-[1-methyl-4-(3-{[1-methyl-4-(1-methylimidazole-2-amido)pyrrol-2-yl]formamido}propanamido)imidazole-2-amido]pyrrole-2-amido}cyclobutaneamido]imidazole-2-carboxylate was obtained as light yellow solid (78.15% yield). LC/MS: mass calcd. For C₃₇H₄₄N₁₄O₈: 812.35, found: 813.50 [M+H]⁺.

### Step 4: Synthesis of 1-methyl-4-[(1r, 3r)-3-{1-methyl-4-[1-methyl-4-(3-{[1-methyl-4-(1-methylimidazole-2-amido)pyrrol-2-yl]formamido}propanamido)imidazole-2-amido]pyrrole-2-amido}cyclobutaneamido]imidazole-2-carboxylic Acid

The procedure was the same as 4-[3-[(tert-butoxycarbonyl)amino] propanamido]-1-methylimidazole-2-carboxylic acid (Example 1 Step 3). 250.00 mg of ethyl 1-methyl-4-[(1r, 3r)-3-{1-methyl-4-[1-methyl-4-(3-{[1-methyl-4-(1-methylimidazole-2-amido)pyrrol-2-yl]formamido}propanamido)imidazole-2-amido]pyrrole-2-amido}cyclobutaneamido]imidazole-2-carboxylate was used, 210.00 mg of 1-methyl-4-[(1r, 3r)-3-{1-methyl-4-[1-methyl-4-(3-{[1-methyl-4-(1-methylimidazole-2-amido)pyrrol-2-yl]formamido}propanamido)imidazole-2-amido]pyrrole-2-amido}cyclobutaneamido]imidazole-2-carboxylic acid was obtained as light yellow solid (87.00% yield).

LC/MS: mass calcd. For C₃₅H₄₀N₁₄O₈: 784.32, found: 785.40 [M+H]⁺.

### Example 8. Synthesis of 1-Methyl-4-(1-{1-methyl-4-[1-methyl-4-(3-{[1-methyl-4-(1-methylimidazole-2-amido)pyrrol-2-yl]formamido]propanamido)imidazole-2-amido]pyrrole-2-carbonyl}azetidine-3-amido)imidazole-2-carboxylic acid (PA-048-P6AZ)

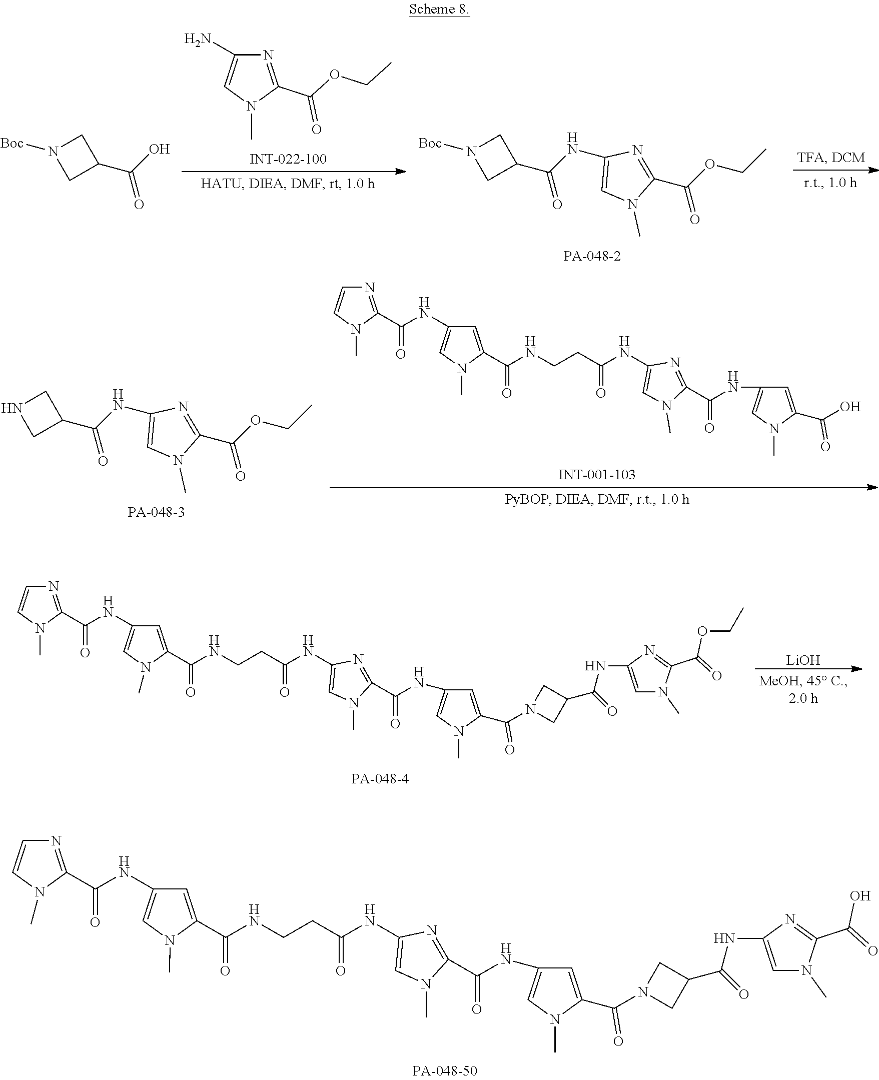

### Step 1: Synthesis of ethyl 4-[1-(tert-butoxycarbonyl)azetidine-3-amido]-1-methylimidazole-2-carboxylate

The procedure was the same as methyl 3-[(4-[3-[(tert-butoxycarbonyl)amino] propanamido]-1-methylimidazol-2-yl)formamido]propanoate (Example 1 Step 6). 300.00 mg of ethyl 4-amino-1-methylimidazole-2-carboxylate was used, 450.00 mg of ethyl 4-[1-(tert-butoxycarbonyl)azetidine-3-amido]-1-methylimidazole-2-carboxylate was obtained as white solid (72.02% yield). LC/MS: mass calcd. For C₁₆H₂₄N₄O₅:352.17, found: 353.10 [M+H]⁺.

### Step 2: Synthesis of ethyl 4-(azetidine-3-amido)-1-methylimidazole-2-carboxylate

The procedure was the same as methyl 4-[4-(4-amino-1-methylpyrrole-2-amido)-1-methylimidazole-2-amido]-1-methylpyrrole-2-carboxylate (Example 2 Step 4). 140.00 mg of ethyl 4-[1-(tert-butoxycarbonyl)azetidine-3-amido]-1-methylimidazole-2-carboxylate was used, 140.00 mg crude of ethyl 4-(azetidine-3-amido)-1-methylimidazole-2-carboxylate was obtained as yellow oil. LC/MS: mass calcd. For C₁₁H₁₆N₄O₃: 252.12, found: 253.10 [M+H]⁺.

### Step 3: Synthesis of ethyl 1-methyl-4-(1-{1-methyl-4-[1-methyl-4-(3-{[1-methyl-4-(1-methylimidazole-2-amido)pyrrol-2-yl]formamido}propanamido)imidazole-2-amido]pyrrole-2-carbonyl}azetidine-3-amido)imidazole-2-carboxylate

The procedure was the same as methyl 4-(4-{4-[(tert-butoxycarbonyl)amino]-1-methylpyrrole-2-amido}-1-methylimidazole-2-amido)-1-methylpyrrole-2-carboxylate (Example 2 Step 3). 140.00 mg of ethyl 4-(azetidine-3-amido)-1-methylimidazole-2-carboxylate was used, 260.00 mg of ethyl 1-methyl-4-(1-{1-methyl-4-[1-methyl-4-(3-{[1-methyl-4-(1-methylimidazole-2-amido)pyrrol-2-yl]formamido}propanamido)imidazole-2-amido]pyrrole-2-carbonyl}azetidine-3-amido)imidazole-2-carboxylate was obtained as white solid (53.96% yield). LC/MS: mass calcd. For C₃₆H₄₂N₁₄O₈: 798.33, found: 799.45. [M+H]⁺.

### Step 4: Synthesis of 1-methyl-4-(1-{1-methyl-4-[1-methyl-4-(3-{[1-methyl-4-(1-methylimidazole-2-amido)pyrrol-2-yl]formamido}propanamido)imidazole-2-amido]pyrrole-2-carbonyl}azetidine-3-amido)imidazole-2-carboxylic Acid

The procedure was the same as 4-[3-[(tert-butoxycarbonyl)amino] propanamido]-1-methylimidazole-2-carboxylic acid (Example 1 Step 3). 250.00 mg of ethyl 1-methyl-4-(1-{1-methyl-4-[1-methyl-4-(3-{[1-methyl-4-(1-methylimidazole-2-amido)pyrrol-2-yl]formamido}propanamido)imidazole-2-amido]pyrrole-2-carbonyl}azetidine-3-amido)imidazole-2-carboxylate was used, 160.00 mg of 1-methyl-4-(1-{1-methyl-4-[1-methyl-4-(3-{[1-methyl-4-(1-methylimidazole-2-amido)pyrrol-2-yl]formamido}propanamido)imidazole-2-amido]pyrrole-2-carbonyl}azetidine-3-amido)imidazole-2-carboxylic acid was obtained as yellow solid (62.08% yield). LC/MS: mass calcd. For C₃₄H₃₈N₁₄O₈: 770.30, found: 771.60 [M+H]⁺.

### Example 9. Synthesis of 3-({1-methyl-4-[3-({4-[1-methyl-4-(3-{[1-methyl-4-(1-methylimidazole-2-amido)pyrrol-2-yl]formamido]propanamido)imidazole-2-amido]-1-[14-(2,5,8,11-tetraoxatridecan-13-yl)-2,5,8,11-tetraoxa-14-azanonadecan-19-yl]pyrrol-2-yl}formamido)propanamido]imidazol-2-yl}formamido)propanoic acid (PA-035-Bis(mPEG4)

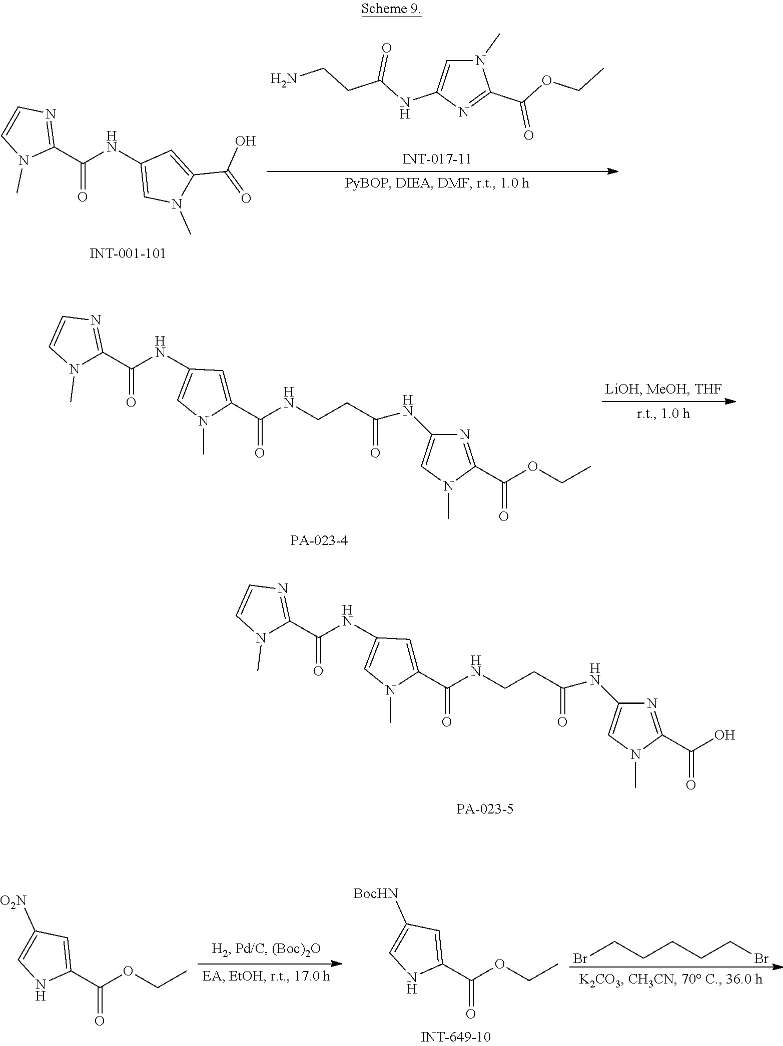

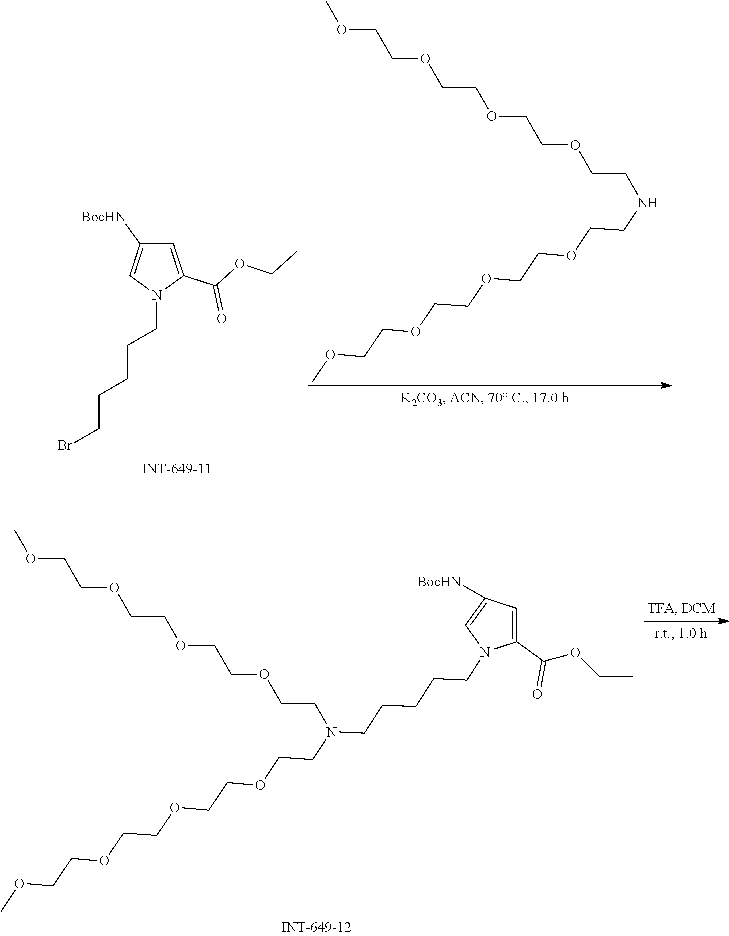 [SMILES: CCOC(=O)c1cc(NC(=O)OC(C)(C)C)cn1CCCCCBr ; COCCOCCOCCOCCNCCOCCOCCOCCOC ; CCOC(=O)c1cc(NC(=O)OC(C)(C)C)cn1CCCCCN(CCOCCOCCOCCOC)CCOCCOCCOCCOC]

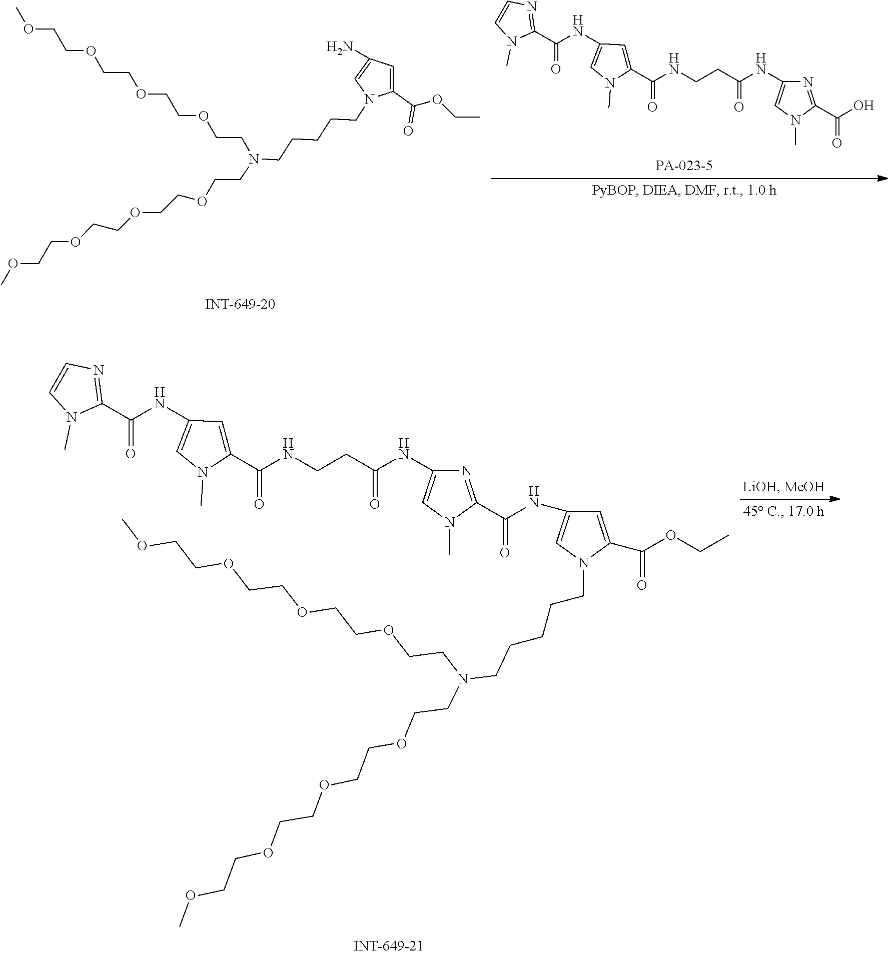

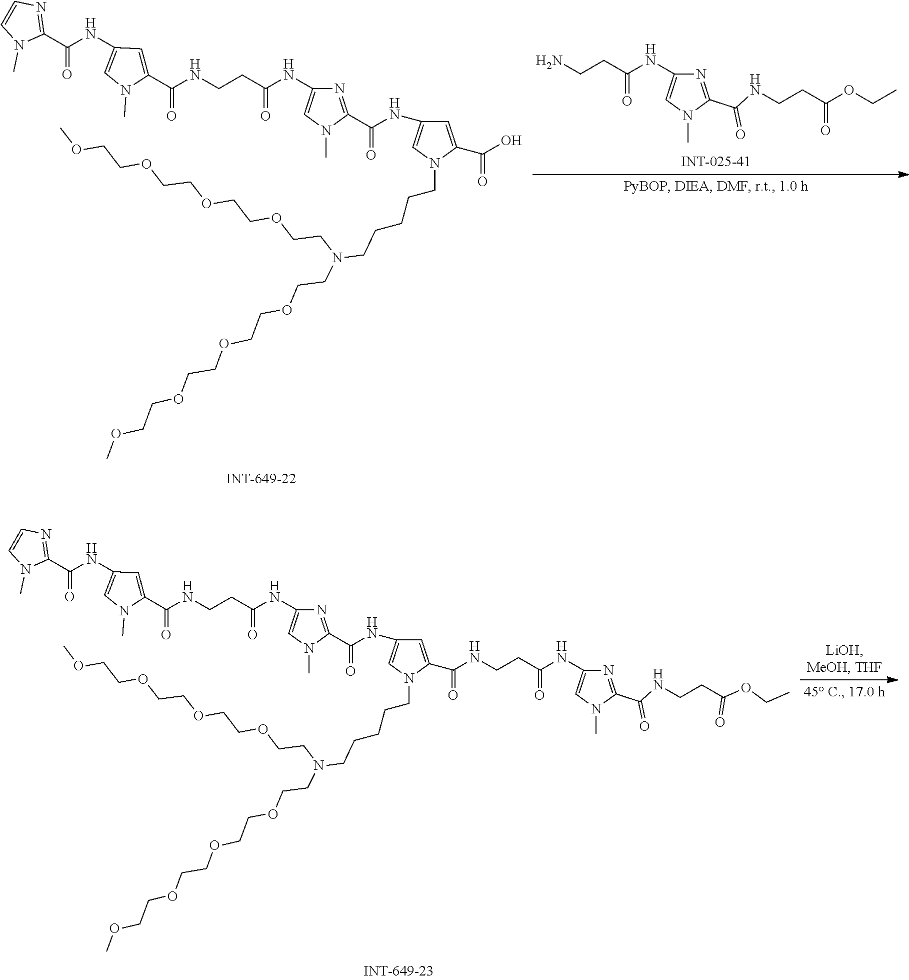

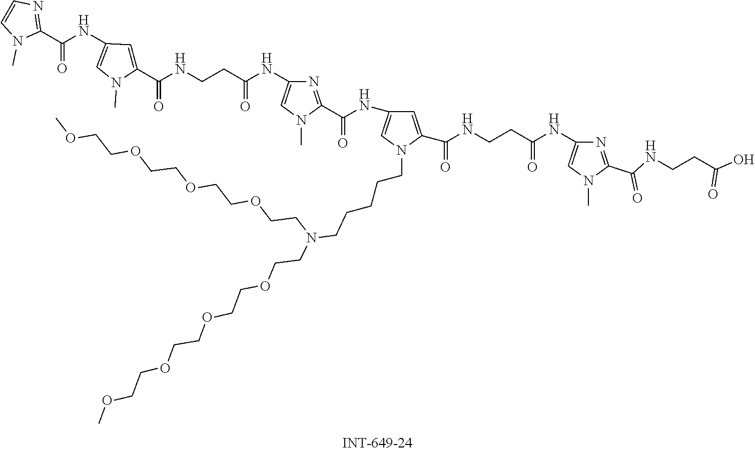

### Step 1: Synthesis of ethyl 1-methyl-4-(3-{[1-methyl-4-(1-methylimidazole-2-amido)pyrrol-2-yl]formamido}propanamido)imidazole-2-carboxylate

The procedure was the same as methyl 4-(4-{4-[(tert-butoxycarbonyl)amino]-1-methylpyrrole-2-amido}-1-methylimidazole-2-amido)-1-methylpyrrole-2-carboxylate (Example 2 Step 3). 2.00 g of 1-methyl-4-(1-methylimidazole-2-amido)pyrrole-2-carboxylic acid was used, 4.00 g of ethyl 1-methyl-4-(3-{[1-methyl-4-(1-methylimidazole-2-amido)pyrrol-2-yl]formamido}propanamido)imidazole-2-carboxylate was obtained as off-white solid. LC/MS: mass calcd. For C₂₁H₂₆N₈O₅: 470.20, found: 471.15 [M+H]⁺.

### Step 2: Synthesis of ethyl 1-methyl-4-(3-{[1-methyl-4-(1-methylimidazole-2-amido)pyrrol-2-yl]formamido}propanamido)imidazole-2-carboxylate

The procedure was the same as 4-[3-[(tert-butoxycarbonyl)amino] propanamido]-1-methylimidazole-2-carboxylic acid (Example 1 Step 3), but the reaction solvent was MeOH/THF (2:1), the reaction temperature was room temperature and the reaction time was 1.0 h. 4.00 g of ethyl 1-methyl-4-(3-{[1-methyl-4-(1-methylimidazole-2-amido)pyrrol-2-yl]formamido}propanamido)imidazole-2-carboxylate was used, 2.85 g of 1-methyl-4-(3-{[1-methyl-4-(1-methylimidazole-2-amido)pyrrol-2-yl]formamido}propanamido)imidazole-2-carboxylic acid was obtained as off-white solid. LC/MS: mass calcd. For C₁₉H₂₂N₈O₅: 442.17, found: 443.30 [M+H]⁺.

### Step 3: Synthesis of ethyl 4-[(tert-butoxycarbonyl)amino]-1H-pyrrole-2-carboxylate

To a solution of ethyl 4-nitro-1H-pyrrole-2-carboxylate (5.00 g, 27.15 mmol, 1.00 equiv) in EtOH (50.00 mL) and EA (50.00 mL) was added Pd/C (1.00 g, 20% w/w) and (Boc)₂0 (11.85 g, 54.304 mmol, 2.00 equiv). Then the reaction was stirred for 17.0 h at room temperature under H₂ atmosphere. The mixture was filtrated and the filtrate was concentrated to afford ethyl 4-[(tert-butoxycarbonyl)amino]-1H-pyrrole-2-carboxylate (5.00 g, 71.54% yield) as white solid. LC/MS: mass calcd. For C₁₂H₁₈N₂O₄: 254.13, found: 255.25 [M+H]⁺.

### Step 4: Synthesis of ethyl 1-(5-bromopentyl)-4-[(tert-butoxycarbonyl)amino] pyrrole-2-carboxylate

To a solution of ethyl 4-[(tert-butoxycarbonyl)amino]-1H-pyrrole-2-carboxylate (4.00 g, 15.73 mmol, 1.00 equiv) in MeCN (70.00 mL) was added K₂CO₃ (6.52 g, 47.19 mmol, 3.00 equiv) and 1,5-dibromopentane (36.17 g, 157.30 mmol, 10.00 equiv). Then the reaction was stirred for 36.0 h at 70 degrees C. The reaction mixture was filtered and the filtrate was concentrated. The residue was purified by silica gel column chromatography, eluted with PE/EA (5:1) to afford ethyl 1-(5-bromopentyl)-4-[(tert-butoxycarbonyl)amino]pyrrole-2-carboxylate (4.82 g, 67.43% yield) as orange solid. LC/MS: mass calcd. For C₁₇H₂₇BrN₂O₄: 402.12, found: 403.00 [M+H]⁺.

### Step 5: Synthesis of ethyl 4-[(tert-butoxycarbonyl)amino]-1-[14-(2,5,8,11-tetraoxatridecan-13-yl)-2,5,8,11-tetraoxa-14-azanonadecan-19-yl]pyrrole-2-carboxylate

The procedure was the same as ethyl 1-(5-bromopentyl)-4-[(tert-butoxycarbonyl)amino]pyrrole-2-carboxylate (Example 9 Step 4), but the reaction time was 17.0 h and the crude product was purified by reverse phase column. 1.00 g of ethyl 1-(5-bromopentyl)-4-[(tert-butoxycarbonyl)amino]pyrrole-2-carboxylate was used, 1.00 g of ethyl 4-[(tert-butoxycarbonyl)amino]-1-[14-(2,5,8,11-tetraoxatridecan-13-yl)-2,5,8,11-tetraoxa-14-azanonadecan-19-yl]pyrrole-2-carboxylate was obtained as brown-yellow oil (61.29% yield). LC/MS: mass calcd. For C₃₅H₆₅N₃O₁₂: 719.46, found: 720.45 [M+H]⁺.

### Step 6: Synthesis of ethyl 4-amino-1-[14-(2,5,8,11-tetraoxatridecan-13-yl)-2,5,8,11-tetraoxa-14-azanonadecan-19-yl]pyrrole-2-carboxylate

The procedure was the same as methyl 4-[4-(4-amino-1-methylpyrrole-2-amido)-1-methylimidazole-2-amido]-1-methylpyrrole-2-carboxylate (Example 2 Step 4). 350.00 mg of ethyl 4-[(tert-butoxycarbonyl)amino]-1-[14-(2,5,8,11-tetraoxatridecan-13-yl)-2,5,8,11-tetraoxa-14-azanonadecan-19-yl]pyrrole-2-carboxylate was used, 350.00 mg crude of ethyl 4-amino-1-[14-(2,5,8,11-tetraoxatridecan-13-yl)-2,5,8,11-tetraoxa-14-azanonadecan-19-yl]pyrrole-2-carboxylate was obtained as yellow oil. LC/MS: mass calcd. For C₃₀H₅₇N₃O₁₀: 619.40, found: 620.60 [M+H]⁺.

### Step 7: Synthesis of ethyl 4-[1-methyl-4-(3-{[1-methyl-4-(1-methylimidazole-2-amido)pyrrol-2-yl]formamido}propanamido)imidazole-2-amido)-1-[14-(2,5,8,11-tetraoxatridecan-13-yl)-2,5,8,11-tetraoxa-14-azanonadecan-19-yl]pyrrole-2-carboxylate

The procedure was the same as methyl 1-methyl-4-(1-methyl-4-{1-methyl-4-[1-methyl-4-(1-methylimidazole-2-amido)pyrrole-2-amido]pyrrole-2-amido}imidazole-2-amido)pyrrole-2-carboxylate (INT-450-11). 220.00 mg of 1-methyl-4-(3-{[1-methyl-4-(1-methylimidazole-2-amido)pyrrol-2-yl]formamido}propanamido)imidazole-2-carboxylic acid was used, 460.00 mg of ethyl 4-[1-methyl-4-(3-{[1-methyl-4-(1-methylimidazole-2-amido)pyrrol-2-yl]formamido}propanamido)imidazole-2-amido]-1-[14-(2,5,8,11-tetraoxatridecan-13-yl)-2,5,8,11-tetraoxa-14-azanonadecan-19-yl]pyrrole-2-carboxylate was obtained as yellow oil (88.59% yield). LC/MS: mass calcd. For C₄₉H₇₇Nn₁O₁₄: 1043.57, found: 1044.60 [M+H]⁺.

### Step 8: Synthesis of 4-[1-methyl-4-(3-{[1-methyl-4-(1-methylimidazole-2-amido)pyrrol-2-yl]formamido}propanamido)imidazole-2-amido)-1-[14-(2,5,8,11-tetraoxatridecan-13-yl)-2,5,8,11-tetraoxa-14-azanonadecan-19-yl]pyrrole-2-carboxylic Acid

The procedure was the same as 1-methyl-4-[3-methyl-3-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-carboxylic acid, but the reaction time was 17.0 h. 450.00 mg of ethyl 4-[1-methyl-4-(3-{[1-methyl-4-(1-methylimidazole-2-amido)pyrrol-2-yl]formamido}propanamido)imidazole-2-amido]-1-[14-(2,5,8,11-tetraoxatridecan-13-yl)-2,5,8,11-tetraoxa-14-azanonadecan-19-yl]pyrrole-2-carboxylate was used, 400.00 mg of 4-[1-methyl-4-(3-{[1-methyl-4-(1-methylimidazole-2-amido)pyrrol-2-yl]formamido}propanamido)imidazole-2-amido]-1-[14-(2,5,8,11-tetraoxatridecan-13-yl)-2,5,8,11-tetraoxa-14-azanonadecan-19-yl]pyrrole-2-carboxylic acid was obtained as yellow oil (91.34% yield). LC/MS: mass calcd. For C₄₇H₇₃N₁₁O₁₄: 1015.53, found: 1039.05[M+Na]+.

### Step 9: Synthesis of ethyl 3-({1-methyl-4-[3-({4-[1-methyl-4-(3-{[1-methyl-4-(1-methylimidazole-2-amido)pyrrol-2-yl]formamido}propanamido)imidazole-2-amido)-1-[14-(2,5,8,11-tetraoxatridecan-13-yl)-2,5,8,11-tetraoxa-14-azanonadecan-19-yl]pyrrol-2-yl}formamido)propanamido)imidazol-2-yl}formamido)propanoate

The procedure was the same as methyl 1-methyl-4-(1-methyl-4-{1-methyl-4-[1-methyl-4-(1-methylimidazole-2-amido)pyrrole-2-amido]pyrrole-2-amido}imidazole-2-amido)pyrrole-2-carboxylate (INT-450-11). 390.00 mg of 4-[1-methyl-4-(3-{[1-methyl-4-(1-methylimidazole-2-amido)pyrrol-2-yl]formamido}propanamido)imidazole-2-amido]-1-[14-(2,5,8,11-tetraoxatridecan-13-yl)-2,5,8,11-tetraoxa-14-azanonadecan-19-yl]pyrrole-2-carboxylic acid was used, 180.00 mg of ethyl 3-({1-methyl-4-[3-({4-[1-methyl-4-(3-{[1-methyl-4-(1-methylimidazole-2-amido)pyrrol-2-yl]formamido}propanamido)imidazole-2-amido]-1-[14-(2,5,8,11-tetraoxatridecan-13-yl)-2,5,8,11-tetraoxa-14-azanonadecan-19-yl]pyrrol-2-yl}formamido)propanamido]imidazol-2-yl}formamido)propanoate was obtained as yellow oil (35.82% yield). LC/MS: mass calcd. For C₆₀H₉₂N₁₆O₁₇: 1308.68, found: 1309.90 [M+H]⁺.

### Step 10: Synthesis of 3-({1-methyl-4-[3-({4-[1-methyl-4-(3-{[1-methyl-4-(1-methylimidazole-2-amido)pyrrol-2-yl]formamido}propanamido)imidazole-2-amido]-1-[14-(2,5,8,11-tetraoxatridecan-13-yl)-2,5,8,11-tetraoxa-14-azanonadecan-19-yl]pyrrol-2-yl}formamido)propanamido]imidazol-2-yl}formamido)propanoic acid (PA-035-Bis(mPEG4))

The procedure was the same as 1-methyl-4-[3-methyl-3-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazole-2-carboxylic acid, but the reaction solvent was MeOH/THF (1:1) and the reaction time was 17.0 h. 170.00 mg of ethyl 3-({1-methyl-4-[3-({4-[1-methyl-4-(3-{[1-methyl-4-(1-methylimidazole-2-amido)pyrrol-2-yl]formamido}propanamido)imidazole-2-amido]-1-[14-(2,5,8,11-tetraoxatridecan-13-yl)-2,5,8,11-tetraoxa-14-azanonadecan-19-yl]pyrrol-2-yl}formamido)propanamido]imidazol-2-yl}formamido)propanoate was used, 80.00 mg of 3-({1-methyl-4-[3-({4-[1-methyl-4-(3-{[1-methyl-4-(1-methylimidazole-2-amido)pyrrol-2-yl]formamido}propanamido)imidazole-2-amido]-1-[14-(2,5,8,11-tetraoxatridecan-13-yl)-2,5,8,11-tetraoxa-14-azanonadecan-19-yl]pyrrol-2-yl}formamido)propanamido]imidazol-2-yl}formamido)propanoic acid was obtained as yellow oil (48.09% yield). LC/MS: mass calcd. For C₅₈H₈₈N₁₆O₁₇: 1280.65, found: 641.90 [M/2+H]⁺.

### Example 10. Synthesis of 3-{[4-(3-{[1-(6-azidohexyl)-4-[1-methyl-4-(3-{[1-methyl-4-(1-methylimidazole-2-amido)pyrrol-2-yl]formamido}propanamido]imidazole-2-amido]pyrrol-2-yl]formamido]propanamido)-1-methylimidazol-2-yl]formamido}propanoic acid (PA-001-P5C6N3)

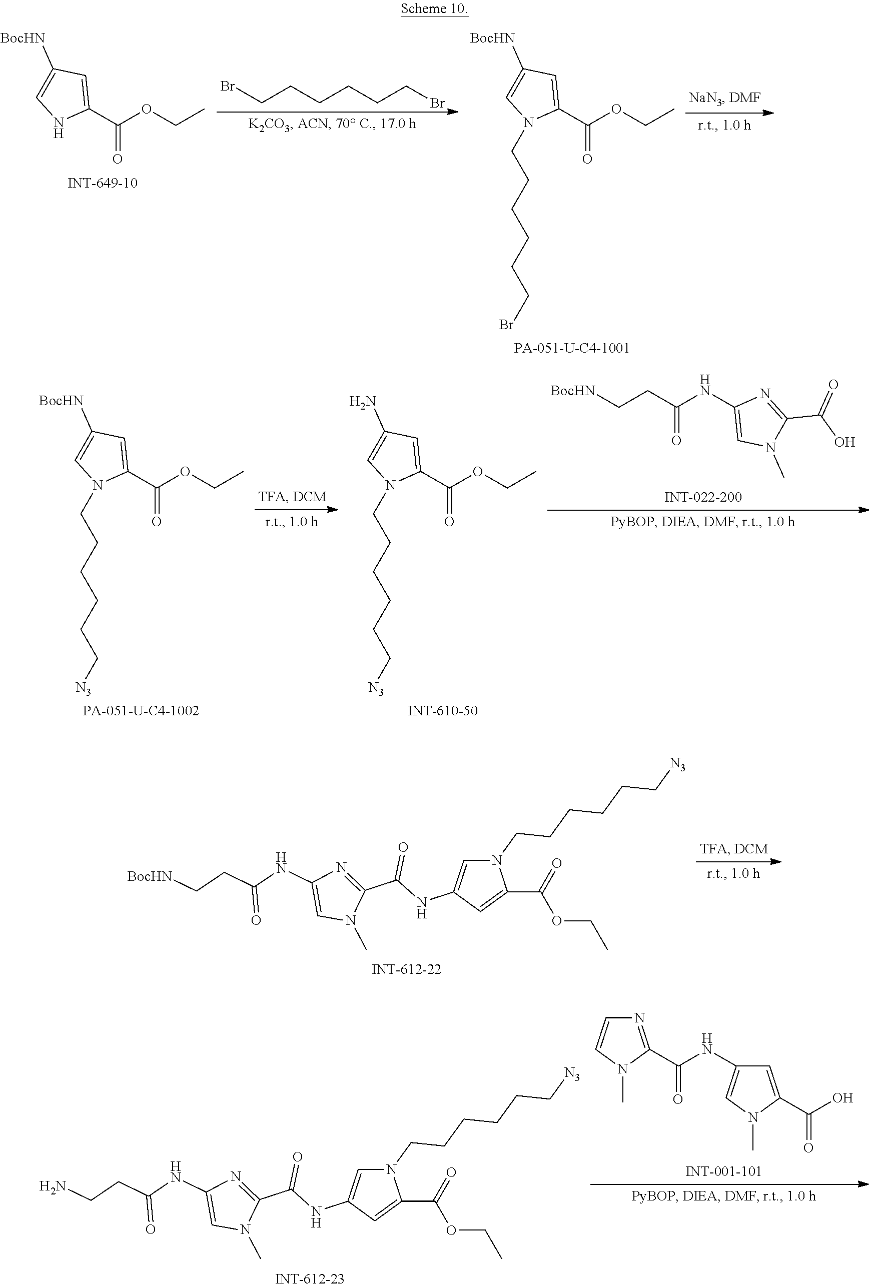

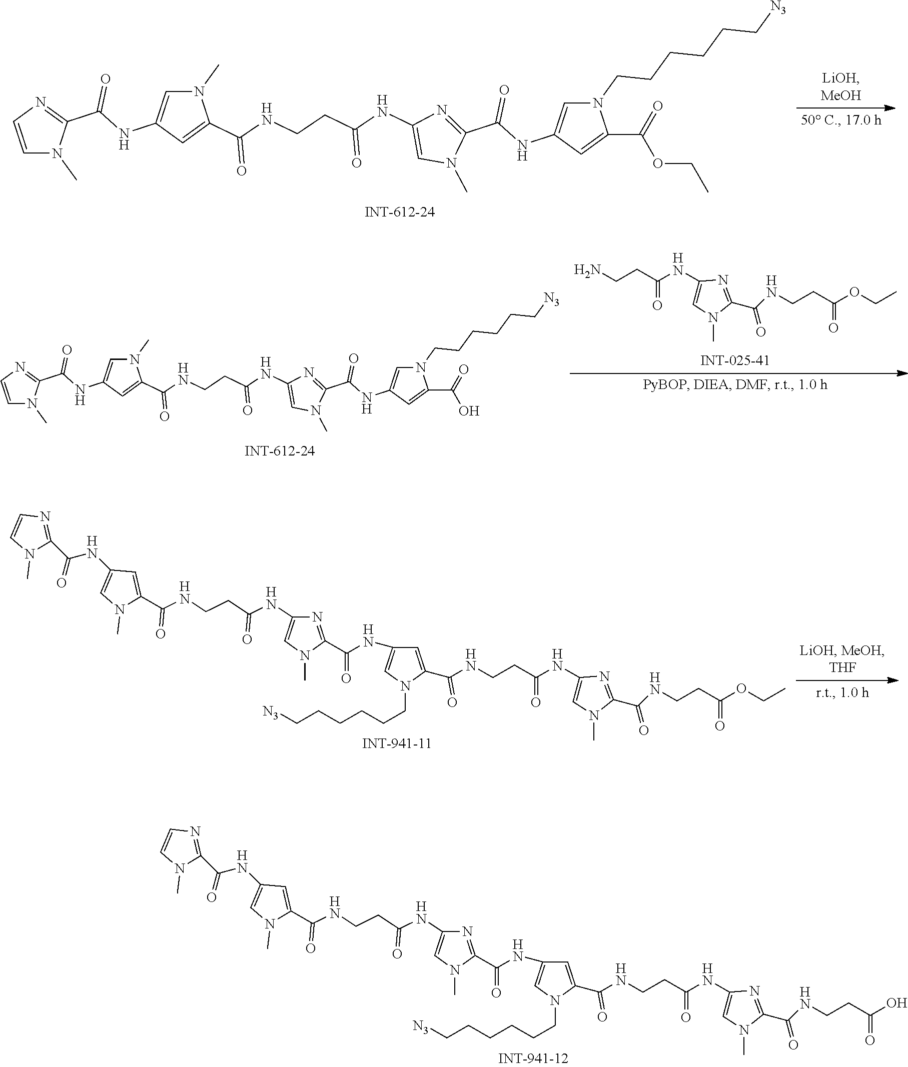 [SMILES: CCOC(=O)c1cc(NC(=O)c2nc(NC(=O)CCNC(=O)c3cc(NC(=O)c4nccn4C)cn3C)cn2C)cn1CCCCCCN=[N+]=[N-] ; Cn1cc(NC(=O)c2nccn2C)cc1C(=O)NCCC(=O)Nc1cn(C)c(C(=O)Nc2cc(C(=O)O)n(CCCCCCN=[N+]=[N-])c2)n1 ; CCOC(=O)CCNC(=O)c1nc(NC(=O)CCN)cn1C ; CCOC(=O)CCNC(=O)c1nc(NC(=O)CCNC(=O)c2cc(NC(=O)c3nc(NC(=O)CCNC(=O)c4cc(NC(=O)c5nccn5C)cn4C)cn3C)cn2CCCCCCN=[N+]=[N-])cn1C ; Cn1cc(NC(=O)c2nccn2C)cc1C(=O)NCCC(=O)Nc1cn(C)c(C(=O)Nc2cc(C(=O)NCCC(=O)Nc3cn(C)c(C(=O)NCCC(=O)O)n3)n(CCCCCCN=[N+]=[N-])c2)n1]

### Step 1: Synthesis of ethyl 1-(6-bromohexyl)-4-[(tert-butoxycarbonyl) amino]pyrrole-2-carboxylate

The procedure was the same as ethyl 1-(5-bromopentyl)-4-[(tert-butoxycarbonyl)amino]pyrrole-2-carboxylate (Example 9 Step 4), but the reaction time was 17.0 h. 8.10 g of ethyl 4-[(tert-butoxycarbonyl)amino]-1H-pyrrole-2-carboxylate was used, 10.70 g of ethyl 1-(6-bromohexyl)-4-[(tert-butoxycarbonyl)amino]pyrrole-2-carboxylate was obtained as white solid (80.49% yield). LC/MS: mass calcd. For C₁₈H₂₉BrN₂O₄: 416.13, found: 417.20, 419.20 [M+H, M+2+H]⁺.

### Step 2: Synthesis of ethyl 1-(6-azidohexyl)-4-[(tert-butoxycarbonyl)amino] pyrrole-2-carboxylate

Into a 250 mL flask was added ethyl 1-(6-bromohexyl)-4-[(tert-butoxycarbonyl)amino]pyrrole-2-carboxylate (10.48 g, 25.11 mmol, 1.00 equiv), DMF (100.00 mL), NaN₃ (2.15 g, 33.15 mmol, 1.32 equiv), the reaction was stirred at room temperature for 1.0 h. The reaction was quenched by the addition of sat. NH₄Cl (aq.) (100 mL) at 0° C. The resulting mixture was extracted with EtOAc (3×100 mL). The combined organic layers were washed with water (1×100 mL), NaCl solution (1×100 mL), dried over anhydrous Na₂SO₄. After filtration, the filtrate was concentrated under reduced pressure. This resulted in ethyl 1-(6-azidohexyl)-4-[(tert-butoxycarbonyl)amino]pyrrole-2-carboxylate (9.00 g, crude) as yellow oil. LC/MS: mass calcd. For C₁₈H₂₉N₅O₄: 379.22, found: 402.40 [M+Na]⁺.

### Step 3: Synthesis of ethyl 4-amino-1-(6-azidohexyl)pyrrole-2-carboxylate

The procedure was the same as methyl 4-[4-(4-amino-1-methylpyrrole-2-amido)-1-methylimidazole-2-amido]-1-methylpyrrole-2-carboxylate (Example 2 Step 4). 300.00 mg of ethyl 1-(6-azidohexyl)-4-[(tert-butoxycarbonyl)amino] pyrrole-2-carboxylate was used, 300.00 mg crude of ethyl 4-amino-1-(6-azidohexyl)pyrrole-2-carboxylate was obtained as yellow oil. LC/MS: mass calcd. For C₁₃H₂₁N₅O₂: 279.17, found: 280.35 [M+H]⁺.

### Step 4: Synthesis of ethyl 1-(6-azidohexyl)-4-(4-[3-[(tert-butoxycarbonyl)amino]propanamido}-1-methylimidazole-2-amido)pyrrole-2-carboxylate

The procedure was the same as ethyl 4-(1-methylimidazole-2-amido)-1H-pyrrole-2-carboxylate (INT-459-2). 300.00 mg of ethyl 4-amino-1-(6-azidohexyl)pyrrole-2-carboxylate was used, 345.00 mg of ethyl 1-(6-azidohexyl)-4-(4-{3-[(tert-butoxycarbonyl)amino]propanamido}-1-methylimidazole-2-amido)pyrrole-2-carboxylate was obtained as pink solid (56.00% yield). LC/MS: mass calcd. For C₂₆H₃₉N₉O₆ Exact Mass: 573.30, found: 574.55 [M+H]⁺.

### Step 5: Synthesis of ethyl 1-(6-azidohexyl)-4-(4-{3-[(tert-butoxycarbonyl) amino]propanamido}-1-methylimidazole-2-amido)pyrrole-2-carboxylate

The procedure was the same as methyl 4-[4-(4-amino-1-methylpyrrole-2-amido)-1-methylimidazole-2-amido]-1-methylpyrrole-2-carboxylate (Example 2 Step 4). 340.00 mg of ethyl 1-(6-azidohexyl)-4-(4-{3-[(tert-butoxycarbonyl)amino]propanamido}-1-methylimidazole-2-amido)pyrrole-2-carboxylate was used, 340.00 mg crude of ethyl 4-[4-(3-aminopropanamido)-1-methylimidazole-2-amido]-1-(6-azidohexyl)pyrrole-2-carboxylate was obtained as yellow oil (56.00% yield). LC/MS: mass calcd. For C₂₁H₃₁N₉O₄ Exact Mass: 473.25, found: 474.40 [M+H]⁺.

### Step 6: Synthesis of ethyl 1-(6-azidohexyl)-4-[1-methyl-4-(3-{[1-methyl-4-(1-methylimidazole-2-amido)pyrrol-2-yl]formamido}propanamido)imidazole-2-amido]pyrrole-2-carboxylate

The procedure was the same as ethyl 1-methyl-4-[3-methyl-3-({1-methyl-4-[1-methyl-4-(3-{[1-methyl-4-(1-methylimidazole-2-amido)pyrrol-2-yl] formamido}propanamido)imidazole-2-amido]pyrrol-2-yl}formamido)butanamido]imidazole-2-carboxylate (Example 6 Step 3). 140.00 mg of 1-methyl-4-(1-methylimidazole-2-amido)pyrrole-2-carboxylic acid was used, 320.00 mg of ethyl 1-(6-azidohexyl)-4-[1-methyl-4-(3-{[1-methyl-4-(1-methylimidazole-2-amido)pyrrol-2-yl]formamido}propanamido)imidazole-2-amido]pyrrole-2-carboxylate was obtained as yellow oil (80.62% yield). LC/MS: mass calcd. For C₃₂H₄₁N₁₃O₆: 703.33, found: 704.55 [M+H]⁺.

### Step 7: Synthesis of 1-(6-azidohexyl)-4-[1-methyl-4-(3-{[1-methyl-4-(1-methylimidazole-2-amido)pyrrol-2-yl]formamido}propanamido)imidazole-2-amido]pyrrole-2-carboxylic Acid

The procedure was the same as 4-[3-[(tert-butoxycarbonyl)amino] propanamido]-1-methylimidazole-2-carboxylic acid (Example 1 Step 3), but the reaction temperature was 50 degrees C. and the reaction time was 17.0 h. 310.00 mg of ethyl 1-(6-azidohexyl)-4-[1-methyl-4-(3-{[1-methyl-4-(1-methylimidazole-2-amido)pyrrol-2-yl]formamido}propanamido)imidazole-2-amido]pyrrole-2-carboxylate was used, 270.00 mg of 1-(6-azidohexyl)-4-[1-methyl-4-(3-{[1-methyl-4-(1-methylimidazole-2-amido)pyrrol-2-yl]formamido}propanamido)imidazole-2-amido]pyrrole-2-carboxylic acid was obtained as yellow solid (90.90% yield). LC/MS: mass calcd. For C₃₀H₃₇N₁₃O₆ Exact Mass: 675.30, found: 676.60 [M+H]⁺.

### Step 8: Synthesis of ethyl 3-{[4-(3-{[1-(6-azidohexyl)-4-[1-methyl-4-(3-{[1-methyl-4-(1-methylimidazole-2-amido)pyrrol-2-yl]formamido}propanamido)imidazole-2-amido]pyrrol-2-yl]formamido}propanamido)-1-methylimidazol-2-yl]formamido}propanoate

The procedure was the same as methyl 4-(4-{4-[(tert-butoxycarbonyl)amino]-1-methylpyrrole-2-amido}-1-methylimidazole-2-amido)-1-methylpyrrole-2-carboxylate (Example 2 Step 3). 200.00 mg of 1-(6-azidohexyl)-4-[1-methyl-4-(3-{[1-methyl-4-(1-methylimidazole-2-amido)pyrrol-2-yl]formamido}propanamido)imidazole-2-amido]pyrrole-2-carboxylic acid was used, 290.00 mg crude of ethyl 3-{[4-(3-{[1-(6-azidohexyl)-4-[1-methyl-4-(3-{[1-methyl-4-(1-methylimidazole-2-amido)pyrrol-2-yl]formamido}propanamido)imidazole-2-amido]pyrrol-2-yl]formamido}propanamido)-1-methylimidazol-2-yl]formamido}propanoate was obtained as white solid. LC/MS: mass calcd. For C₄₃H₅₆N₁₈O₉: 968.45, found: 969.30 [M+H]⁺.

### Step 9: Synthesis of 3-{[4-(3-{[1-(6-azidohexyl)-4-[1-methyl-4-(3-{[1-methyl-4-(1-methylimidazole-2-amido)pyrrol-2-yl]formamido}propanamido)imidazole-2-amido]pyrrol-2-yl]formamido}propanamido)-1-methylimidazol-2-yl]formamido}propanoic Acid

The procedure was the same as 4-[3-[(tert-butoxycarbonyl)amino] propanamido]-1-methylimidazole-2-carboxylic acid (Example 1 Step 3), but the reaction solvent was MeOH/THF (1:1), the reaction temperature was room temperature and the reaction time was 1.0 h. 280.00 mg of ethyl 3-{[4-(3-{[1-(6-azidohexyl)-4-[1-methyl-4-(3-{[1-methyl-4-(1-methylimidazole-2-amido)pyrrol-2-yl]formamido}propanamido)imidazole-2-amido]pyrrol-2-yl]formamido}propanamido)-1-methylimidazol-2-yl]formamido}propanoate was used, 240.00 mg of 3-{[4-(3-{[1-(6-azidohexyl)-4-[1-methyl-4-(3-{[1-methyl-4-(1-methylimidazole-2-amido)pyrrol-2-yl]formamido}propanamido)imidazole-2-amido]pyrrol-2-yl]formamido}propanamido)-1-methylimidazol-2-yl]formamido}propanoic acid was obtained as white solid (88.27% yield). LC/MS: mass calcd. For C₄₁H₅₂N₁₈O₉: 940.42, found: 941.20 [M+H]⁺

### Synthesis of Representative Ligands

### Example 11. Synthesis of 4-[2-(4-fluoro-2,6-dimethylphenoxy)-5-(2-hydroxypropan-2-yl)phenyl]-6-methyl-7-oxo-1H-pyrrolo[2,3-c]pyridine-2-carboxylic Acid

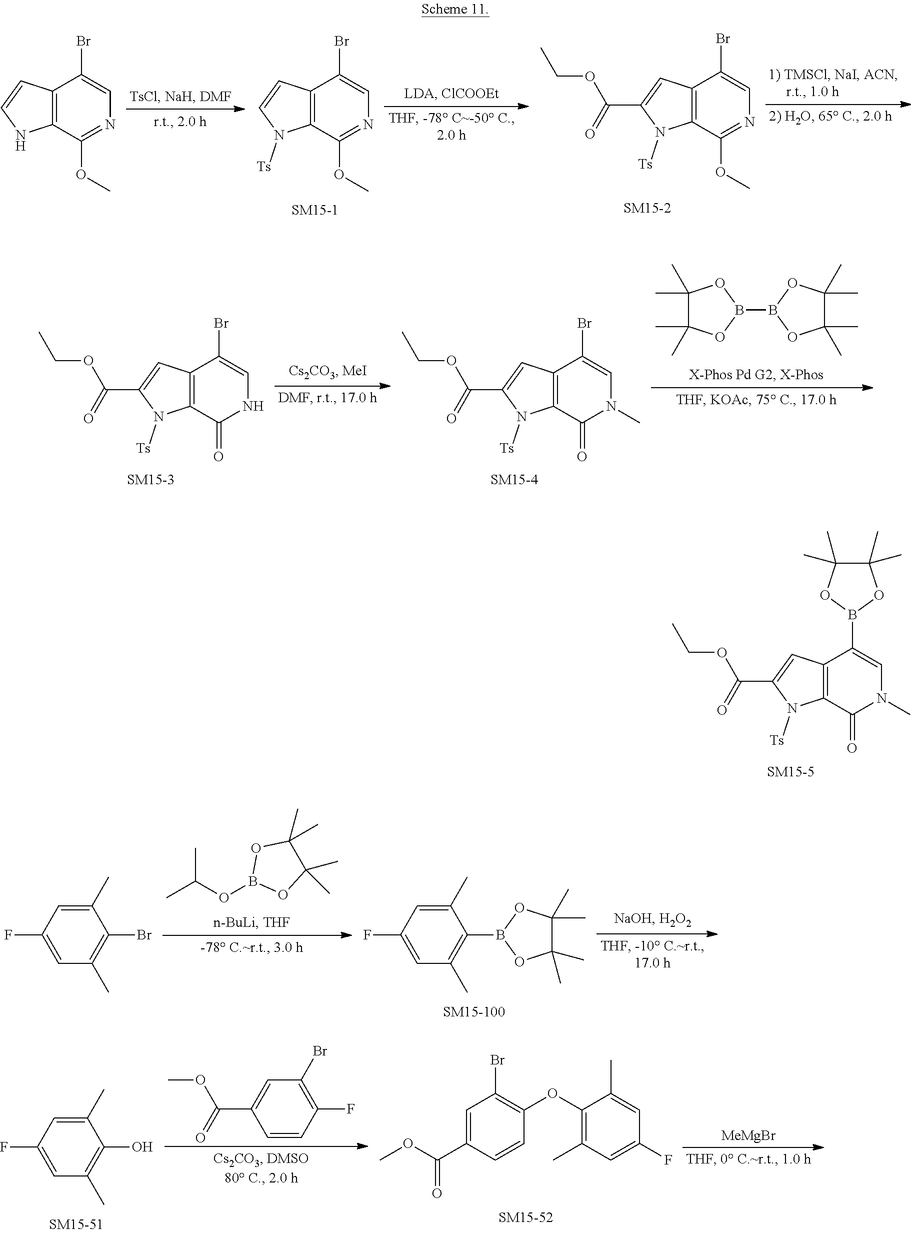

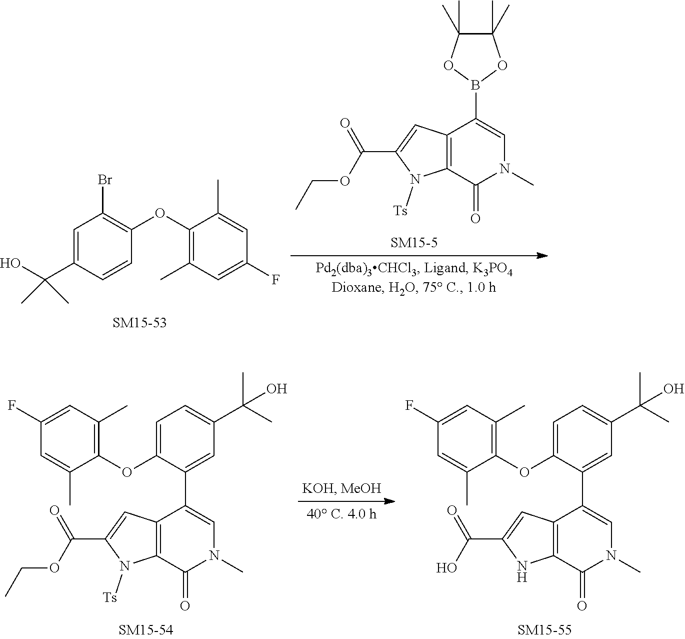 [SMILES: Cc1cc(F)cc(C)c1Oc1ccc(C(C)(C)O)cc1Br ; CCOC(=O)c1cc2c(B3OC(C)(C)C(C)(C)O3)cn(C)c(=O)c2n1S(=O)(=O)c1ccc(C)cc1 ; CCOC(=O)c1cc2c(-c3cc(C(C)(C)O)ccc3Oc3c(C)cc(F)cc3C)cn(C)c(=O)c2n1S(=O)(=O)c1ccc(C)cc1 ; Cc1cc(F)cc(C)c1Oc1ccc(C(C)(C)O)cc1-c1cn(C)c(=O)c2[nH]c(C(=O)O)cc12]

### Step 1: Synthesis of 4-bromo-7-methoxy-1-(4-methylbenzenesulfonyl)pyrrolo [2,3-c]pyridine

To a solution of 4-bromo-7-methoxy-1H-pyrrolo[2,3-c]pyridine (5.00 g, 22.12 mmol, 1.00 equiv) in DMF (20.00 mL) was added NaH (60%, 796.46 mg, 33.19 mmol, 1.50 equiv) in portions at 0 degrees C. Then the reaction was stirred for 15.0 min followed by addition of TsCl (6.30 g, 33.19 mmol, 1.50 equiv) at 0 degrees C. The resulting mixture was stirred for additional 2.0 h at room temperature. The mixture was poured into ice and water (60 mL). The solid was filtrated out, washed with H₂O (10 mL) and dried to afford 4-bromo-7-methoxy-1-(4-methylbenzenesulfonyl)pyrrolo[2,3-c] pyridine (7.50 g, 83.98% yield) as white solid. LCMS: mass calcd. For C₁₅H₁₃BrN₂O₃S: 379.98, found: 380.95, 382.95 [M+H, M+2+H]⁺.

### Step 2: Synthesis of ethyl 4-bromo-7-methoxy-1-tosyl-1H-pyrrolo[2,3-c]pyridine-2-carboxylate

To a solution of 4-bromo-7-methoxy-1-tosyl-1H-pyrrolo[2,3-c]pyridine (6.30 g, 16.58 mmol, 1.00 equiv) in THF (80.00 mL) was added LDA (2M in THF, 12.50 mL, 24.87 mmol, 1.50 equiv) dropwise at −78 degrees C. and the mixture stirred at −78 degrees C. to −50 degrees C. for 1.0 h, followed by dropwise addition of CICOOEt (2.69 g, 24.87 mmol, 1.50 equiv). After 2.0 h, the reaction mixture was quenched with saturated NH₄Cl (aq), and the residue was extracted with EA (3×300 mL). The organic phases were combined and dried over Na₂SO₄, filtrated and concentrated. The residue was purified by silica gel column chromatography, eluted with PE/EA=10:1 to afford ethyl 4-bromo-7-methoxy-1-tosyl-1H-pyrrolo[2,3-c]pyridine-2-carboxylate (7.00 g, 90.11% yield) as white solid. LCMS: mass calcd. For C₁₈H17BrN₂O₅S: 452.00, found: 453.00, 455.00 [M+H, M+2+H]⁺.

### Step 3: Synthesis of Ethyl 4-bromo-7-oxo-1-tosyl-6,7-dihydro-1H-pyrrolo[2,3-c]pyridine-2-carboxylate

To a stirred solution of ethyl 4-bromo-7-methoxy-1-tosyl-1H-pyrrolo[2,3-c] pyridine-2-carboxylate (4.00 g, 8.850 mmol, 1.00 equiv) in CH₃CN (80.00 mL) was added TMSCl (1.45 g, 13.28 mmol, 1.50 equiv) and NaI (2.00 g, 13.28 mmol, 1.50 equiv) in portions at room temperature under N₂ atmosphere. The mixture was stirred for 1.0 h at room temperature, then H₂O (238.95 mg, 13.28 mmol, 1.50 equiv) was added dropwise at 65 degrees C. The mixture was stirred for 2.0 h at 65 degrees C. The reaction mixture was cooled to room temperature. The precipitate was filtered, washed with water (50 mL), dried over vacuum. Ethyl 4-bromo-7-oxo-1-tosyl-6,7-dihydro-1H-pyrrolo[2,3-c]pyridine-2-carboxylate (4.30 g, crude) was obtained as brown solid. LCMS: mass calcd. For C₁₇H₁₅BrN₂O₅S: 437.99, found: 438.95, 440.95 [M+H, M+2+H]⁺.

### Step 4: Synthesis of ethyl 4-bromo-6-methyl-1-(4-methylbenzenesulfonyl)-7-oxopyrrolo[2,3-c]pyridine-2-carboxylate

To a solution of ethyl 4-bromo-7-oxo-1-tosyl-6,7-dihydro-1H-pyrrolo[2,3-c] pyridine-2-carboxylate (4.30 g, 9.82 mmol, 1.00 equiv) in DMF (20.00 mL) was added C₈₂CO₃ (3.83 g, 11.78 mmol, 1.20 equiv), Mel (1.67 g, 11.78 mmol, 1.20 equiv) was added dropwise into this reaction. The reaction mixture was stirred for 17.0 h at room temperature under N₂ atmosphere. The mixture was poured into ice water (60 mL). The solid was filtrated out, washed with H₂O (10 mL) and dried to afford ethyl 4-bromo-6-methyl-1-(4-methylbenzenesulfonyl)-7-oxopyrrolo[2,3-c]pyridine-2-carboxylate (4.30 g, crude) as brown solid. LCMS: mass calcd. For C₁₈H₁₇BrN₂O₅S: 452.00, found: 453.15, 455.15 [M+H, M+2+H]⁺.

### Step 5: Synthesis of ethyl 6-methyl-1-(4-methylbenzenesulfonyl)-7-oxo-4-(4,4,5,5-tetramethyl-1,3,2-dioxaborolan-2-yl)pyrrolo[2,3-c]pyridine-2-carboxylate

To a solution of ethyl 4-bromo-6-methyl-1-(4-methylbenzenesulfonyl)-7-oxopyrrolo[2,3-c]pyridine-2-carboxylate (1.00 g, 2.21 mmol, 1.00 equiv) in THF (30.00 mL) was added bis(pinacolato)diboron (1.12 g, 4.41 mmol, 2.00 equiv), KOAc (650.00 mg, 6.62 mmol, 3.00 equiv), X-Phos Pd G2 (175.00 mg, 0.22 mmol, 0.10 equiv) and X-Phos (106.00 mg, 0.22 mmol, 0.10 equiv) at room temperature under N₂ atmosphere. The resulting mixture was stirred for 17.0 h at 75 degrees C. under N₂ atmosphere. The mixture was concentrated, 40 mL H₂O was added to the residue, then the mixture was extracted with EA (3×40 mL), the organic phases were combined and washed with NaCl solution (40 mL), dried over Na₂SO₄. The solid was filtrated out and the filtrate was concentrated. Ethyl 6-methyl-1-(4-methylbenzenesulfonyl)-7-oxo-4-(4,4,5,5-tetramethyl-1,3,2-dioxaborolan-2-yl)pyrrolo[2,3-c]pyridine-2-carboxylate (2.20 g, crude) was obtained as yellow solid. The crude was used for next step directly. LCMS: mass calcd. For C₂₁H₂₅BN₂O₅S: 500.18, found: 501.10 [M+H]⁺.

### Step 6: Synthesis of 2-(4-fluoro-2,6-dimethylphenyl)-4,4,5,5-tetramethyl-1,3,2-dioxaborolane

A solution of 2-bromo-5-fluoro-1,3-dimethylbenzene (5.00 g, 24.62 mmol, 1.00 equiv) in THF(15.00 mL) was added n-BuLi (2.5 M, 14.77 mL, 36.94 mmol, 1.50 equiv) dropwise at −78 degrees C. under N₂ atmosphere. The mixture was stirred for 3.0 h at −78 degrees C., then 2-isopropoxy-4,4,5,5-tetramethyl-1,3,2-dioxaborolane (6.87 g, 36.94 mmol, 1.50 equiv) was added dropwise at −78 degrees C. The resulting mixture was warmed to room temperature naturally and stirred for 3.0 h. After reaction, the reaction was quenched with water (20 mL) at 0 degrees C. The resulting mixture was extracted with EtOAc (3×20 mL). The combined organic layers were dried over anhydrous Na₂SO₄. After filtration, the filtrate was concentrated under reduced pressure to afford 2-(4-fluoro-2,6-dimethylphenyl)-4,4,5,5-tetramethyl-1,3,2-dioxaborolane (6.70 g, crude) as a light yellow oil. The crude product was used in the next step directly without further purification. LC/MS: mass calcd. For C₁₄H₂₀BFO₂: 250.15, found: 251.30 [M+1]+.

### Step 7: Synthesis of 4-fluoro-2,6-dimethylphenol

To a stirred solution of 2-(4-fluoro-2,6-dimethylphenyl)-4,4,5,5-tetramethyl-1,3,2-dioxaborolane (6.70 g, 26.79 mmol, 1.00 equiv) in THF (20.00 mL) were added NaOH (1.61 g, 40.25 mmol, 1.50 equiv) and H₂O₂(9.99 mL, 428.59 mmol, 16.00 equiv) dropwise at −10 degrees C. under N₂ atmosphere. The resulting mixture was stirred for 17.0 h at room temperature. After reaction, the mixture was acidified to pH=1 with HCl (aq. 2M). The resulting mixture was extracted with EtOAc (3×20 mL). The combined organic layers were washed with saturated NaHCO₃ (aq.) (1×10 mL) and saturated Na₂S₂O₃(aq.) (1×10 mL), dried over anhydrous Na₂SO₄. After filtration, the filtrate was concentrated under reduced pressure. The residue was purified by silica gel column chromatography, eluted with hexane/EtOAc (12:1) to afford 4-fluoro-2,6-dimethylphenol (2.70 g, 66.88% yield) as white solid. ¹HNMR (400 MHz, DMSO) 5: 8.12 (s, 1H), 6.73 (d, J=9.3 Hz, 2H), 2.16 (s, 6H).

### Step 8: Synthesis of methyl3-bromo-4-(4-fluoro-2,6-dimethylphenoxy)benzoate

To a stirred solution of 4-fluoro-2,6-dimethylphenol (2.70 g, 19.26 mmol, 1.00 equiv) and methyl 3-bromo-4-fluorobenzoate (4.94 g, 21.20 mmol, 1.10 equiv) in DMSO (20.00 mL) were added C₈₂CO₃ (9.41 g, 28.90 mmol, 1.50 equiv) at room temperature. The resulting mixture was stirred for 2.0 h at 80 degrees C. After reaction, the reaction was quenched by the addition of water (30 mL) at room temperature. The resulting mixture was extracted with EtOAc (3×20 mL). The combined organic layers were combined and dried over anhydrous Na₂SO₄. After filtration, the filtrate was concentrated under reduced pressure. The residue was purified by silica gel column chromatography, eluted with hexane/EtOAc (12:1) to afford methyl 3-bromo-4-(4-fluoro-2,6-dimethylphenoxy)benzoate (6.80 g, 94.95% yield) as white solid. LC/MS: mass calcd. For C₁₆H₁₄BrFO₃: 352.01, found: 353.15 [M+H]⁺.

### Step 9: Synthesis of 2-[3-bromo-4-(4-fluoro-2,6-dimethylphenoxy)phenyl]propan-2-ol

To a stirred solution of methyl 3-bromo-4-(4-fluoro-2,6-dimethylphenoxy) benzoate (2.00 g, 5.66 mmol, 1.00 equiv) in THF (10.00 mL) were added bromo (methyl)magnesium (3.00 M in 2-Me-THF, 11.33 mL, 33.98 mmol, 6.00 equiv) at 0 degrees C. under N₂ atmosphere. The resulting mixture was stirred for 1.0 h at 0 degrees C. under N₂ atmosphere. After reaction, the reaction was quenched by the addition of sat. NH₄Cl (aq.) (10 mL) at 0 degrees C. The resulting mixture was extracted with EtOAc (3×10 mL). The combined organic layers were combined and dried over anhydrous Na₂SO₄. After filtration, the filtrate was concentrated under reduced pressure. The residue was purified by silica gel column chromatography, eluted with hexane/EtOAc (10:1) to afford 2-[3-bromo-4-(4-fluoro-2,6-dimethyl phenoxy)phenyl]propan-2-ol (1.80 g, 77.40% yield) as white solid. LC/MS: mass calcd. For C₁₇H₁₈BrFO₂: 352.05, found: 335.00 [M-OH]⁺.

### Step 10: Synthesis of ethyl4-[2-(4-fluoro-2,6-dimethylphenoxy)-5-(2-hydroxypropan-2-yl)phenyl]-6-methyl-1-(4-methylbenzenesulfonyl)-7-oxopyrrolo[2,3-c]pyridine-2-carboxylate

To a stirred solution of 2-[3-bromo-4-(4-fluoro-2,6-dimethylphenoxy)phenyl] propan-2-ol (500.00 mg, 1.42 mmol, 1.00 equiv) and ethyl 6-methyl-1-(4-methylbenzenesulfonyl)-7-oxo-4-(4,4,5,5-tetramethyl-1,3,2-dioxaborolan-2-yl)pyrrolo[2,3-c]pyridine-2-carboxylate (1.42 g, 2.83 mmol, 2.00 equiv) in dioxane (16.00 mL) and H₂O (4.00 mL) were added Pd₂(dba)₃·CHCl₃ (129.62 mg, 0.14 mmol, 0.10 equiv), K₃PO₄ (901.39 mg, 4.25 mmol, 3.00 equiv) and 1,3,5,7-Tetramethyl-2,4,8-trioxa-6-phenyl-6-phosphaadamantane (82.00 mg, 0.28 mmol, 0.20 equiv) at room temperature under N₂ atmosphere. The resulting mixture was stirred for 1.0 h at 75 degrees C. under N₂ atmosphere. The reaction was quenched with water at room temperature. The resulting mixture was extracted with EtOAc (3×20 mL). The combined organic layers were dried over anhydrous Na₂SO₄. After filtration, the filtrate was concentrated under reduced pressure. The residue was purified by silica gel column chromatography, eluted with hexane/EtOAc (1:1) to afford ethyl 4-[2-(4-fluoro-2,6-dimethylphenoxy)-5-(2-hydroxypropan-2-yl)phenyl]-6-methyl-1-(4-methylbenzenesulfonyl)-7-oxopyrrolo[2,3-c]pyridine-2-carboxylate (620.00 mg, 48.76% yield) as white solid. LC/MS: mass calcd. For C₃₅H₃₅FN₂O₇S: 646.21, found: 647.20 [M+H]⁺.

### Step 11: Synthesis of 4-[2-(4-fluoro-2,6-dimethylphenoxy)-5-(2-hydroxypropan-2-yl)phenyl]-6-methyl-7-oxo-1H-pyrrolo[2,3-c]pyridine-2-carboxylic Acid

To a stirred solution/mixture of ethyl 4-[2-(4-fluoro-2,6-dimethylphenoxy)-5-(2-hydroxypropan-2-yl)phenyl]-6-methyl-1-(4-methylbenzenesulfonyl)-7-oxopyrrolo[2,3-c]pyridine-2-carboxylate (600.00 mg, 0.93 mmol, 1.00 equiv) in MeOH (15.00 mL) were added KOH (2M, 3.71 mL, 7.42 mmol, 8.00 equiv) at room temperature. The resulting mixture was stirred for 4.0 h at 40 degrees C. After reaction, the resulting mixture was concentrated under vacuum. Then the residue was dissolved in water (10 mL) and acidified to pH 3 with HCl (2M aq.). The precipitated solids were collected by filtration and washed with water (3×10 mL). The solid was concentrated under vacuum to afford 4-[2-(4-fluoro-2,6-dimethylphenoxy)-5-(2-hydroxypropan-2-yl) phenyl]-6-methyl-7-oxo-1H-pyrrolo[2,3-c]pyridine-2-carboxylic acid (390.00 mg, 64.26% yield) as white solid. LC/MS: mass calcd. For C₂₆H₂₅FN₂O₅: 464.17, found: 465.15 [M+H]⁺.

### Example 12. Synthesis of 1-ethyl-4-[2-(4-fluoro-2,6-dimethylphenoxy)-5-(2-hydroxypropan-2-yl)phenyl]-6-methyl-7-oxopyrrolo[2,3-c]pyridine-2-carboxylic Acid

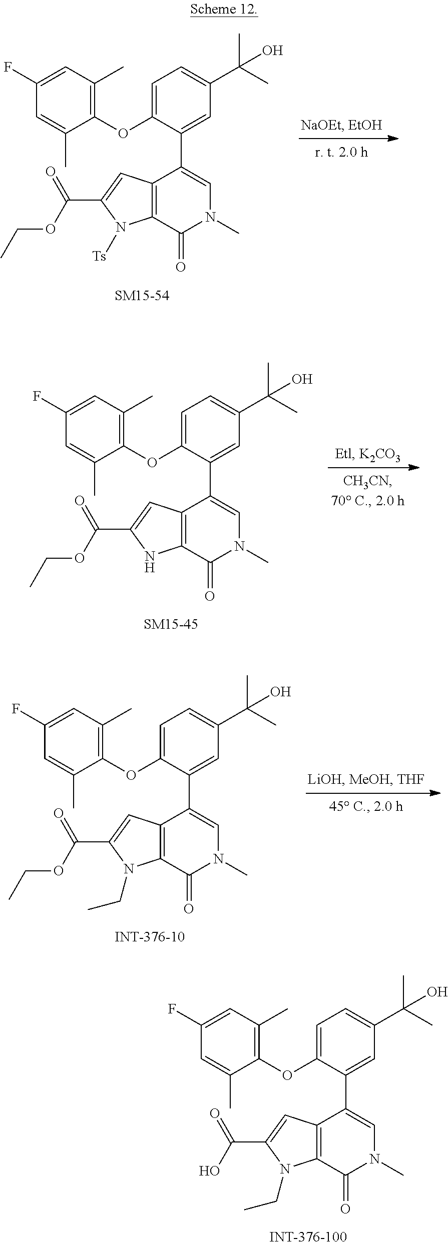 [SMILES: CCOC(=O)c1cc2c(-c3cc(C(C)(C)O)ccc3Oc3c(C)cc(F)cc3C)cn(C)c(=O)c2n1S(=O)(=O)c1ccc(C)cc1 ; CCOC(=O)c1cc2c(-c3cc(C(C)(C)O)ccc3Oc3c(C)cc(F)cc3C)cn(C)c(=O)c2[nH]1 ; CCOC(=O)c1cc2c(-c3cc(C(C)(C)O)ccc3Oc3c(C)cc(F)cc3C)cn(C)c(=O)c2n1CC ; CCn1c(C(=O)O)cc2c(-c3cc(C(C)(C)O)ccc3Oc3c(C)cc(F)cc3C)cn(C)c(=O)c21]

### Step 1: Synthesis of ethyl 4-[2-(4-fluoro-2,6-dimethylphenoxy)-5-(2-hydroxypropan-2-yl)phenyl]-6-methyl-7-oxo-1H-pyrrolo[2,3-c]pyridine-2-carboxylate

To a stirred solution of ethyl 4-[2-(4-fluoro-2,6-dimethylphenoxy)-5-(2-hydroxypropan-2-yl)phenyl]-6-methyl-1-(4-methylbenzenesulfonyl)-7-oxopyrrolo[2,3-c]pyridine-2-carboxylate (3.40 g, 5.26 mmol, 1.00 equiv) in ethyl alcohol (50.00 mL) was added sodium ethoxide (894.40 mg, 13.14 mmol, 2.50 equiv) at room temperature. The resulting mixture was stirred for 2.0 h at room temperature. After reaction, the reaction was poured into citric acid solution (3.32 g, 3.00 equiv, 125 mL). Then the resulting mixture was extracted with EtOAc (3×150 mL). The combined organic layers were washed with water (2×50 mL), dried over anhydrous Na2SO4. After filtration, the filtrate was concentrated under reduced pressure. The residue was washed with diethyl ether (3×10 mL) to afford ethyl 4-[2-(4-fluoro-2,6-dimethylphenoxy)-5-(2-hydroxypropan-2-yl)phenyl]-6-methyl-7-oxo-1H-pyrrolo[2,3-c]pyridine-2-carboxylate (2.00 g, 71.78% yield) as white solid. LCMS: mass calcd. For C₂₈H₂₉FN₂O₅: 492.21, found: 493.40 [M+H]⁺.

### Step 2: Synthesis of ethyl 1-ethyl-4-[2-(4-fluoro-2,6-dimethylphenoxy)-5-(2-hydroxypropan-2-yl)phenyl]-6-methyl-7-oxopyrrolo[2,3-c]pyridine-2-carboxylate

The procedure was the same as ethyl 1-(5-bromopentyl)-4-[(tert-butoxycarbonyl)amino]pyrrole-2-carboxylate, but the reaction time was 2.0 h. 500.00 mg of ethyl 4-[2-(4-fluoro-2,6-dimethylphenoxy)-5-(2-hydroxypropan-2-yl)phenyl]-6-methyl-7-oxo-1H-pyrrolo[2,3-c]pyridine-2-carboxylate was used, 500.00 mg of ethyl 1-ethyl-4-[2-(4-fluoro-2,6-dimethylphenoxy)-5-(2-hydroxypropan-2-yl)phenyl]-6-methyl-7-oxopyrrolo[2,3-c]pyridine-2-carboxylate was obtained as white solid (94.61% yield). LC/MS: mass calcd. For C₃₀H₃₃FN₂O₅: 520.24, found: 521.35 [M+H]⁺.

### Step 3: Synthesis of 1-ethyl-4-[2-(4-fluoro-2,6-dimethylphenoxy)-5-(2-hydroxypropan-2-yl)phenyl]-6-methyl-7-oxopyrrolo[2,3-c]pyridine-2-carboxylic Acid

The procedure was the same as 4-[3-[(tert-butoxycarbonyl)amino] propanamido]-1-methylimidazole-2-carboxylic acid, but the reaction solvent was MeOH/THF (1:5). 500.00 mg of ethyl 1-ethyl-4-[2-(4-fluoro-2,6-dimethylphenoxy)-5-(2-hydroxypropan-2-yl)phenyl]-6-methyl-7-oxopyrrolo[2,3-c]pyridine-2-carboxylate was used, 514.00 mg crude of 1-ethyl-4-[2-(4-fluoro-2,6-dimethylphenoxy)-5-(2-hydroxypropan-2-yl)phenyl]-6-methyl-7-oxopyrrolo[2,3-c]pyridine-2-carboxylic acid was obtained as light yellow solid. LC/MS: mass calcd. For C₂₈H₂₉FN₂O₅: 492.21, found: 493.15 [M+H]⁺.

### Example 13. Synthesis of 4-[2-(4-fluoro-2,6-dimethylphenoxy)-5-methanesulfonylphenyl]-6-methyl-7-oxo-1H-pyrrolo[2,3-c]pyridine-2-carboxylic Acid

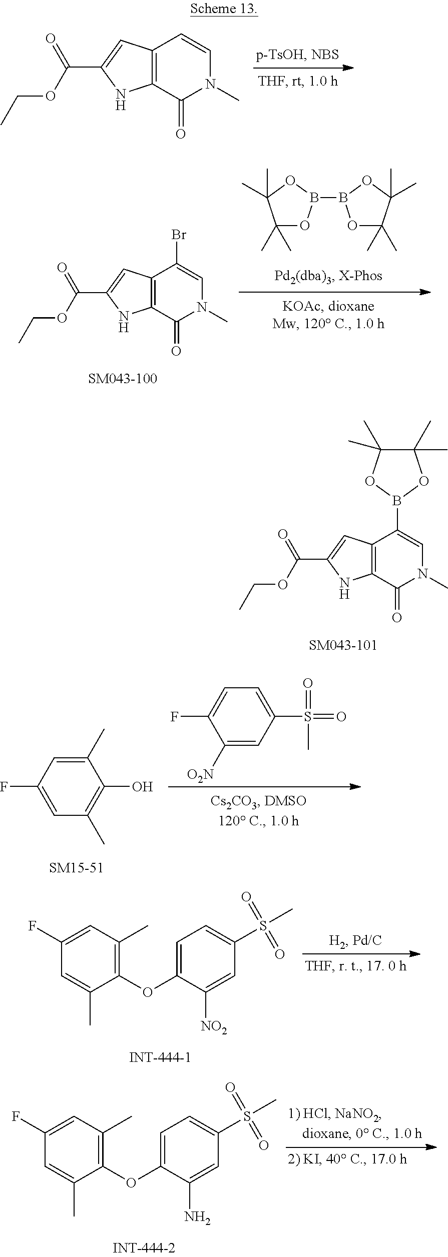

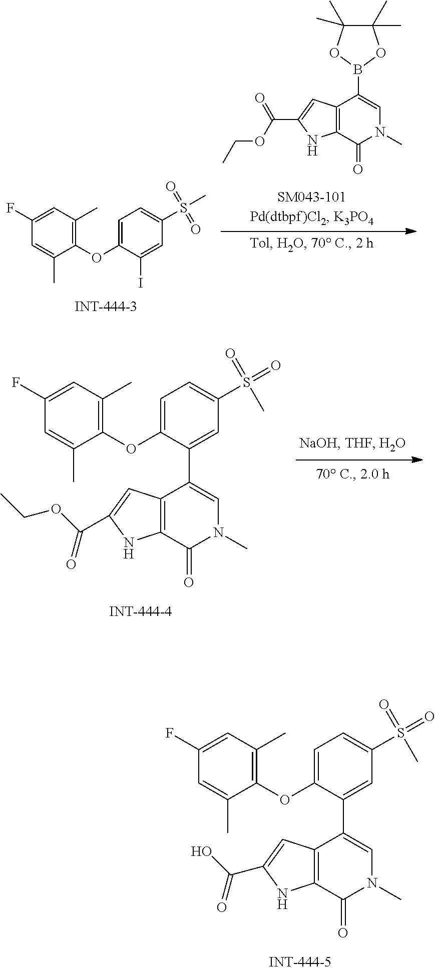

### Step 1: Synthesis of ethyl 4-bromo-6-methyl-7-oxo-1H-pyrrolo[2,3-c]pyridine-2-carboxylate

To a stirred solution of ethyl 6-methyl-7-oxo-1H-pyrrolo[2,3-c]pyridine-2-carboxylate (10.00 g, 45.41 mmol, 1.00 equiv) in tetrahydrofuran (150.00 mL) was added NBS (8.08 g, 45.41 mmol, 1.00 equiv) and p-TsOH (3.91 g, 22.70 mmol, 0.50 equiv). The resulting mixture was stirred at room temperature for 1.0 h. The resulting mixture was concentrated under vacuum. The residue was purified by silica gel column chromatography (0-10% MeOH/DCM) to afford ethyl 4-bromo-6-methyl-7-oxo-1H-pyrrolo[2,3-c]pyridine-2-carboxylate (13.00 g, 95.71% yield) as yellow solid. LC/MS: mass calcd. For C₁₁H₁₁BrN₂O₃: 298.00, found: 299.00, 301.00 [M+H, M+2+H]⁺.

### Step 2: Synthesis of ethyl 6-methyl-7-oxo-4-(4,4,5,5-tetramethyl-1,3,2-dioxaborolan-2-yl)-1H-pyrrolo[2,3-c]pyridine-2-carboxylate

To a stirred solution of ethyl 4-bromo-6-methyl-7-oxo-1H-pyrrolo[2,3-c] pyridine-2-carboxylate (13.00 g, 43.46 mmol, 1.00 equiv) in dioxane (150.00 mL) was added bis(pinacolato)diboron (22.07 g, 86.92 mmol, 2.00 equiv), Pd₂(dba)₃·CHCl₃ (4.00 g, 4.36 mmol, 0.10 equiv) and AcOK (8.53 g, 86.92 mmol, 2.00 equiv). The final reaction mixture was irradiated with microwave radiation for 1.0 h at 120 degrees C. The reaction was proceeded on 1.0 g scale and 13 times were repeated. Then the reaction mixtures were combined and worked up together. 150 mL H₂O was added, the resulting mixture was extracted with EA (3×150 mL). The organic layers were combined, dried over anhydrous sodium sulfate, filtered and concentrated. The residue was purified by silica gel column chromatography (50-70% EA/PE) to afford ethyl 6-methyl-7-oxo-4-(4,4,5,5-tetramethyl-1,3,2-dioxaborolan-2-yl)-1H-pyrrolo[2,3-c]pyridine-2-carboxylate (10.00 g, 66.47% yield) as yellow solid. LC/MS: mass calcd. For C₇H₂₃BN₂O₅: 346.17, found: 347.20 [M+H]⁺.

### Step 3: Synthesis of 5-fluoro-2-(4-methanesulfonyl-2-nitrophenoxy)-1,3-dimethylbenzene

The procedure was the same as methyl 3-bromo-4-(4-fluoro-2,6-dimethylphenoxy)benzoate (SM15-52), but the reaction temperature was 120 degrees C. and the reaction time was 1.0 h. 2.00 g of 4-fluoro-2,6-dimethylphenol was used, 4.60 g of desired product was obtained as off-white solid (94.05% yield). ¹H NMR (300 MHz, DMSO-d6) δ: 8.60 (s, 1H), 8.07 (d, J=9.0 Hz, 1H), 7.16 (d, J=9.0 Hz, 2H), 6.88 (d, J=9.0 Hz, 1H), 3.31 (s, 3H), 2.09 (s, 6H).

### Step 4: Synthesis of 2-(2,4-difluorophenoxy)-5-methanesulfonylaniline

To a stirred solution of 1-(2,4-difluorophenoxy)-4-methanesulfonyl-2-nitrobenzene (500.00 mg, 1.52 mmol, 1.00 equiv) in THF (10.00 mL) was added Pd/C (100.00 mg, 20% w/w). The mixture was hydrogenated at room temperature for 17.0 h under H₂ atmosphere using a hydrogen balloon. The resulting mixture was filtered, the filter cake was washed with EA (3×20 mL). The filtrate was concentrated under reduced pressure to afford 2-(2,4-difluorophenoxy)-5-methanesulfonylaniline (450.00 mg, crude) as light yellow oil. The crude product was used in the next step directly without further purification. LC/MS: mass calcd. C₁₅H₁₆FNO₃S: 309.08 found: 310.10 [M+H]⁺.

### Step 5: Synthesis of 5-fluoro-2-(2-iodo-4-methanesulfonylphenoxy)-1,3-dimethylbenzene

To a stirred solution of 2-(4-fluoro-2,6-dimethylphenoxy)-5-methanesulfonylaniline (500.00 mg, 1.62 mmol, 1.00 equiv) in dioxane (5.00 mL) was added concentrated hydrogen chloride (1.00 mL) dropwise at 0 degrees C. The resulting mixture was stirred for 10.0 min at 0 degrees C. To the above mixture was added sodium nitrite (133.81 mg, 1.94 mmol, 1.20 equiv) at 0 degrees C. The resulting mixture was stirred for additional 1.0 h at 0 degrees C. To the above mixture was added KI (536.60 mg, 3.23 mmol, 2.00 equiv) at 0 degrees C. The resulting mixture was stirred for additional 17.0 h at 40 degrees C. After reaction, the reaction was quenched with water (5 mL) at room temperature. The resulting mixture was extracted with EtOAc (3×10 mL). The combined organic layers were washed with water (1×10 mL), dried over anhydrous Na₂SO₄. After filtration, the filtrate was concentrated under reduced pressure. The residue was purified by Prep-TLC (PE/EA 5:1) to afford 5-fluoro-2-(2-iodo-4-methanesulfonylphenoxy)-1,3-dimethylbenzene (250.00 mg, 31.65% yield) as light yellow oil. LC/MS: mass calcd. C₁₅H₁₄FIO₃S: 419.97, found: 442.95 [M+Na]+.

### Step 6: Synthesis of ethyl 4-[2-(4-fluoro-2,6-dimethylphenoxy)-5-methanesulfonylphenyl]-6-methyl-7-oxo-1H-pyrrolo[2,3-c]pyridine-2-carboxylate

To a stirred solution of 5-fluoro-2-(2-iodo-4-methanesulfonylphenoxy)-1,3-dimethylbenzene (380.00 mg, 0.90 mmol, 1.00 equiv) in toluene (6.00 mL) and water (1.50 mL) was added ethyl 6-methyl-7-oxo-4-(4,4,5,5-tetramethyl-1,3,2-dioxaborolan-2-yl)-1H-pyrrolo[2,3-c]pyridine-2-carboxylate (469.56 mg, 1.36 mmol, 1.50 equiv), K₃PO₄ (383.88 mg, 1.81 mmol, 2.00 equiv) and Pd(dtbpf)Cl₂ (58.93 mg, 0.09 mmol, 0.10 equiv) at room temperature under N₂ atmosphere. The resulting mixture was stirred for 2.0 h at 70 degrees C. under N₂ atmosphere. After reaction, the reaction was quenched with water (10 mL) at room temperature. The resulting mixture was extracted with EtOAc (3×10 mL). The combined organic layers were washed with water (1×5 mL), dried over anhydrous Na₂SO₄. After filtration, the filtrate was concentrated under reduced pressure. The residue was purified by silica gel column chromatography, eluted with PE/EA (0˜100%) to afford ethyl 4-[2-(4-fluoro-2,6-dimethylphenoxy)-5-methanesulfonylphenyl]-6-methyl-7-oxo-1H-pyrrolo[2,3-c]pyridine-2-carboxylate (250.00 mg, 52.86% yield) as dark yellow solid. LC/MS: mass calcd. C₂₆H₂₅FN₂O₆S: 512.14, found: 513.30 [M+H]⁺.

### Step 7: Synthesis of 4-[2-(4-fluoro-2,6-dimethylphenoxy)-5-methanesulfonylphenyl]-6-methyl-7-oxo-JH-pyrrolo[2,3-c]pyridine-2-carboxylic Acid

To a stirred solution of ethyl 4-[2-(4-fluoro-2,6-dimethylphenoxy)-5-methanesulfonylphenyl]-6-methyl-7-oxo-1H-pyrrolo[2,3-c]pyridine-2-carboxylate (240.00 mg, 0.47 mmol, 1.00 equiv) in tetrahydrofuran (1.00 mL) and water (5.00 mL) was added caustic soda (74.91 mg, 1.87 mmol, 4.00 equiv) at room temperature. The resulting mixture was stirred for 2.0 h at 70 degrees C. After reaction, the resulting mixture was concentrated under reduced pressure. The residue was dissolved in water (5 mL). The mixture was acidified to pH 4 with HCl (aq. 2M). The precipitated solids were collected by filtration and washed with water (3×5 mL), dried under vacuum. This resulted in 4-[2-(4-fluoro-2,6-dimethylphenoxy)-5-methanesulfonylphenyl]-6-methyl-7-oxo-1H-pyrrolo[2,3-c]pyridine-2-carboxylic acid (170.00 mg, 73.44% yield) as light yellow solid. LC/MS: mass calcd. For C₂₄H₂₁FN₂O₆S: 484.11, found: 485.10 [M+H]⁺.

### Example 14. Synthesis of 4-[5-(ethanesulfonyl)-2-(4-fluoro-2,6-dimethylphenoxy)phenyl]-6-methyl-7-oxo-1H-pyrrolo[2,3-c]pyridine-2-carboxylic Acid

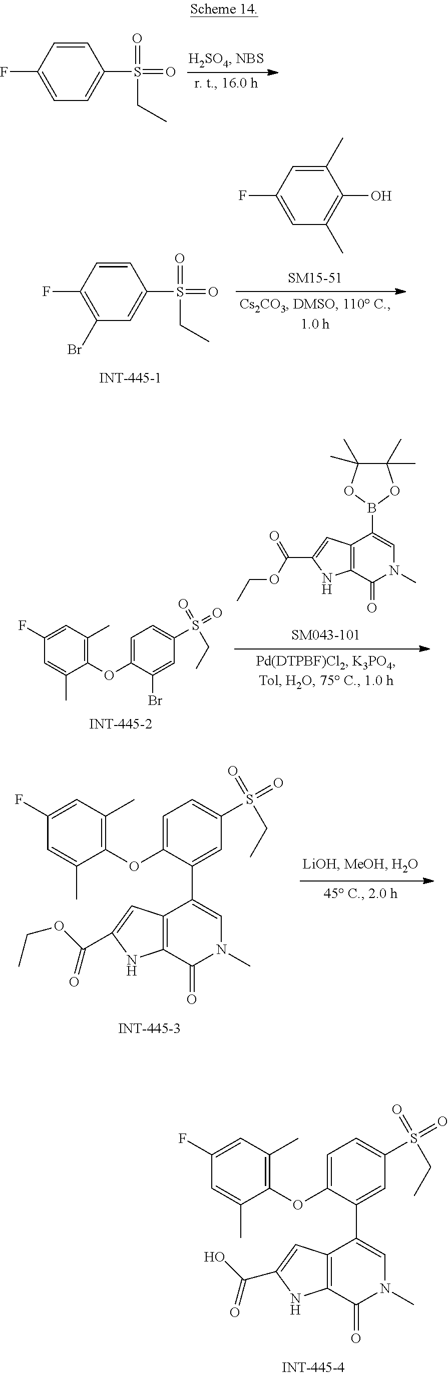

### Step 1: Synthesis of 2-bromo-4-(ethanesulfonyl)-1-fluorobenzene

To a stirred solution of fluoresone (1.00 g, 5.31 mmol, 1.00 equiv) in H₂SO₄ (6.00 mL) was added NBS (1.04 g, 5.84 mmol, 1.10 equiv). The resulting mixture was stirred at room temperature for 16.0 h. The resulting mixture was poured into ice water (20 mL). The precipitated solids were collected by filtration, washed with PE (50 mL) and dried to afford 2-bromo-4-(ethanesulfonyl)-1-fluorobenzene (890.00 mg, 62.71% yield) as yellow solid. LC/MS: mass calcd. For C₈H₈BrFO₂S: 265.94, 267.05, 268.95[M+H, M+H⁺²].

### Step 2: Synthesis of 2-[2-bromo-4-(ethanesulfonyl)phenoxy]-5-fluoro-1,3-dimethylbenzene

The procedure was the same as methyl 3-bromo-4-(4-fluoro-2,6-dimethylphenoxy)benzoate (SM15-52), but the reaction temperature was 110 degrees C., the reaction time was 1.0 h and the crude product was used for next step without purification. 870.00 mg of 1,3-dibromo-5-(ethanesulfonyl)-2-fluorobenzene was used, 950.00 mg of 2-[2-bromo-4-(ethanesulfonyl) phenoxy]-5-fluoro-1,3-dimethylbenzene was obtained as yellow solid (97.56% yield). LC/MS: mass calcd. C₁₆H₁₆BrFO₃S: 386.00, found: 387.05, 389.05 [M+H, M+2+H]⁺.

### Step 3: Synthesis of 2-[2-bromo-4-(ethanesulfonyl)phenoxy]-5-fluoro-1,3-dimethylbenzene

The procedure was the same as ethyl 4-[2-(4-fluoro-2,6-dimethylphenoxy)-5-methanesulfonylphenyl]-6-methyl-7-oxo-1H-pyrrolo[2,3-c]pyridine-2-carboxylate (INT-444-4), but the reaction temperature was 75 degrees C. and the reaction time was 1.0 h. 950.00 mg of 2-[2-bromo-4-(ethanesulfonyl)phenoxy]-5-fluoro-1,3-dimethylbenzene was used, 870.00 mg of ethyl 4-[5-(ethanesulfonyl)-2-(4-fluoro-2,6-dimethylphenoxy)phenyl]-6-methyl-7-oxo-1H-pyrrolo[2,3-c]pyridine-2-carboxylate was obtained as yellow solid (67.35% yield). LC/MS: mass calcd. C₂₇H₂₇FN₂O₆S: 526.15, found: 527.35 [M+H]⁺.

### Step 4: Synthesis of 4-[5-(ethanesulfonyl)-2-(4-fluoro-2,6-dimethylphenoxy) phenyl]-6-methyl-7-oxo-1H-pyrrolo[2,3-c]pyridine-2-carboxylic Acid

The procedure was the same as 4-[3-[(tert-butoxycarbonyl)amino] propanamido]-1-methylimidazole-2-carboxylic acid. 860.00 mg of ethyl 4-[5-(ethanesulfonyl)-2-(4-fluoro-2,6-dimethylphenoxy)phenyl]-6-methyl-7-oxo-1H-pyrrolo[2,3-c]pyridine-2-carboxylate was used, 590.00 mg of 4-[5-(ethanesulfonyl)-2-(4-fluoro-2,6-dimethylphenoxy)phenyl]-6-methyl-7-oxo-1H-pyrrolo[2,3-c]pyridine-2-carboxylic acid was obtained as yellow solid (72.46% yield). LC/MS: mass calcd. C₂₅H₂₃FN₂O₆S: 498.12, found: 499.25 [M+H]⁺.

### Example 15. Synthesis of (R)-2-(4-(4-chlorophenyl)-2,3,9-trimethyl-6H-thieno[3,2-f][1,2,4]triazolo[4,3-a][1,4]diazepin-6-yl)acetic Acid

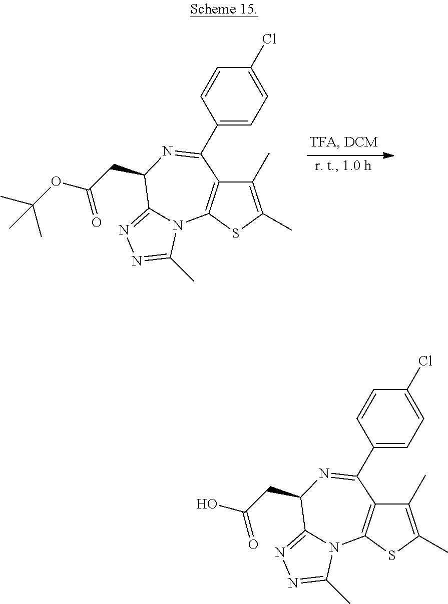 [SMILES: Cc1sc2c(c1C)C(c1ccc(Cl)cc1)=N[C@H](CC(=O)OC(C)(C)C)c1nnc(C)n1-2 ; Cc1sc2c(c1C)C(c1ccc(Cl)cc1)=N[C@H](CC(=O)O)c1nnc(C)n1-2]

The procedure was the same as methyl 4-[4-(4-amino-1-methylpyrrole-2-amido)-1-methylimidazole-2-amido]-1-methylpyrrole-2-carboxylate (Example 2 Step 3). 500.00 mg of tert-butyl (R)-2-(4-(4-chlorophenyl)-2,3,9-trimethyl-6H-thieno[3,2-f][1,2,4]triazolo[4,3-a][1,4]diazepin-6-yl)acetate was used, 500.00 mg crude of desired product was obtained as yellow oil. LC/MS: mass calcd. C₁₉H₁₇ClN₄O₂S: 400.08, found: 400.95 [M+H]⁺.

### Example 16. Synthesis of (S)-2-(4-(4-(16-((2-(1H-indol-3-yl)ethyl)(((9H-fluoren-9-yl)methoxy)carbonyl)amino)hexadecanamido)phenyl)-2,3,9-trimethyl-6H-thieno[3,2-f][1,2,4]triazolo[4,3-a][1,4]diazepin-6-yl)acetic Acid

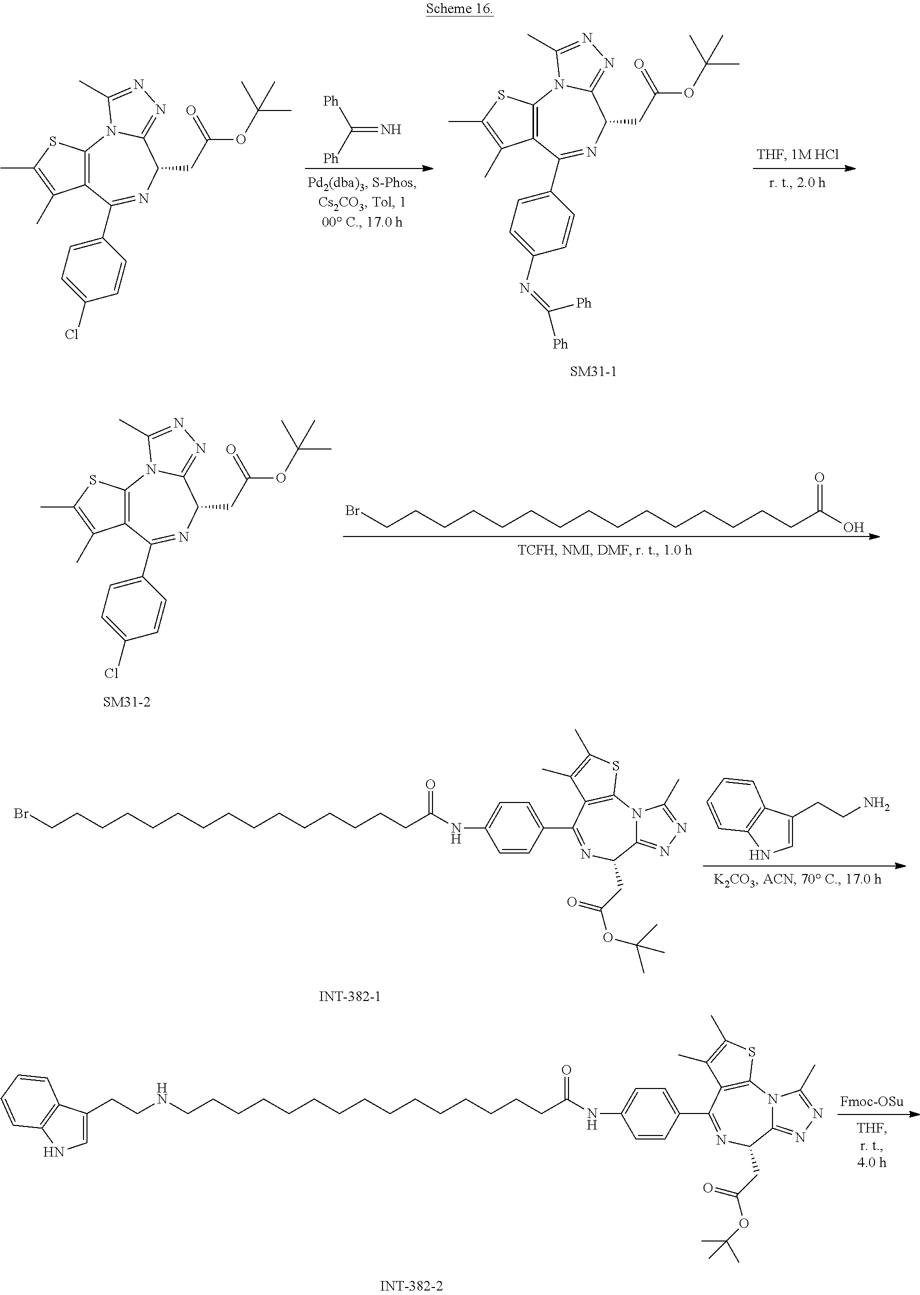

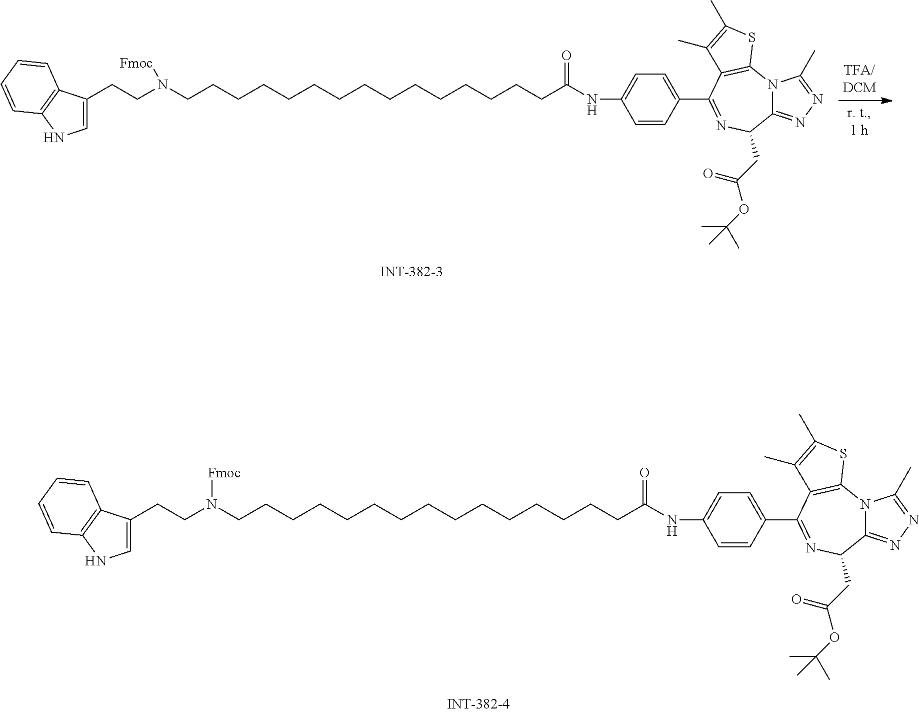

### Step 1: Synthesis of tert-butyl (S)-2-(4-(4-((diphenylmethylene) amino)phenyl)-2,3,9-trimethyl-6H-thieno[3,2-f][1,2,4]triazolo[4,3-a][1,4]diazepin-6-yl)acetate

To a solution of tert-butyl (S)-2-(4-(4-chlorophenyl)-2,3,9-trimethyl-6H-thieno[3,2-f][1,2,4]triazolo[4,3-a][1,4]diazepin-6-yl)acetate (1000.00 mg, 2.19 mmol, 1.00 equiv) in toluene (30.00 mL) was added diphenylmethanimine (594.89 mg, 3.28 mmol, 1.50 equiv), C₈₂CO₃ (2138.91 mg, 6.56 mmol, 3.00 equiv), Pd₂(dba)₃·CHCl₃ (200.38 mg, 0.22 mmol, 0.10 equiv) and S-Phos (89.83 mg, 0.22 mmol, 0.10 equiv) at room temperature under N₂ atmosphere. The resulting mixture was stirred for 17.0 h at 110 degrees C. under N₂ atmosphere. After cooling down to room temperature, the reaction solvent was removed under reduced pressure. The residue obtained was purified by silica gel chromatography (40-50% EA/PE) to afford desired product (1180.00 mg, 80.65% yield) as yellow solid. LC/MS: mass calcd. for C₃₆H₃₅N₅O₂S: 601.25, found: 602.35 [M+H]⁺.

### Step 2: Synthesis of tert-butyl (S)-2-(4-(4-aminophenyl)-2,3,9-trimethyl-6H-thieno[3,2-f][1,2,4]triazolo[4,3-a][1,4]diazepin-6-yl)acetate

To a stirred solution of tert-butyl (S)-2-(4-(4-((diphenylmethylene)amino)phenyl)-2,3,9-trimethyl-6H-thieno[3,2-f][1,2,4]triazolo[4,3-a][1,4]diazepin-6-yl)acetate (1180.00 mg, 1.96 mmol, 1.00 equiv) in THF (10.00 mL) was added 1 M HCl (2.00 mL) dropwise at room temperature. The resulting mixture was stirred for 2.0 h at room temperature. The mixture was diluted with EA (100 mL) and the organic layer was separated. The organic layer was concentrated and purified by Prep-HPLC with the following conditions: Column: CHIRALPAK 1H-3, 3.0*50 mm, 3 μm; Mobile Phase, MeOH (0.1% DEA; Flow rate: 2 mL/min; Gradient: 10% B; 220 n. The fractions were combined and lyophilized directly. Tert-butyl (S)-2-(4-(4-aminophenyl)-2,3,9-trimethyl-6H-thieno[3,2-f][1,2,4]triazolo[4,3-a][1,4]diazepin-6-yl)acetate (600.00 mg, 67.83%) was obtained as white solid. LC/MS: mass calcd. for C₂₃H₂₇N₅O₂S: 437.19, found: 438.10 [M+H]⁺.

### Step 3: Synthesis of tert-butyl (S)-2-(4-(4-(16-bromohexadecanamido)phenyl)-2,3,9-trimethyl-6H-thieno[3,2-f][1,2,4]triazolo[4,3-a][1,4]diazepin-6-yl)acetate

16-bromohexadecanoic acid (191.59 mg, 0.57 mmol, 1.00 equiv) was dissolved in DMF (3.00 mL). NMI (93.82 mg, 1.14 mmol, 2.00 equiv), TCFH (320.62 mg, 1.14 mmol, 2.00 equiv) and tert-butyl (S)-2-(4-(4-aminophenyl)-2,3,9-trimethyl-6H-thieno[3,2-f][1,2,4]triazolo[4,3-a][1,4]diazepin-6-yl)acetate (250.00 mg, 0.57 mmol, 1.00 equiv) were added in turn to the solution at 0 degrees C. The mixture was allowed to warm to room temperature and stirred for 1.0 h at room temperature. After the reaction was completed, the mixture was added to the ice water (7 mL) dropwise. The solid was generated, filtered out, washed by water (2×5 mL), and dried under vacuum to afford tert-butyl (S)-2-(4-(4-(16-bromohexadecanamido)phenyl)-2,3,9-trimethyl-6H-thieno[3,2-f][1,2,4]triazolo[4,3-a][1,4]diazepin-6-yl)acetate (450.00 mg, 90.77%) as light yellow solid. LC/MS: mass calcd. For C₃₉H₅₆BrN₅O₃S: 753.32, found: 754.55, 756.55 [M+H, M+2+H]⁺.

### Step 4: Synthesis of tert-butyl (S)-2-(4-(4-(16-((2-(1H-indol-3-yl)ethyl)amino) hexadecanamido)phenyl)-2,3,9-trimethyl-6H-thieno[3,2-f][1,2,4]triazolo[4,3-a][1,4]diazepin-6-yl)acetate

To a solution of tert-butyl (S)-2-(4-(4-(16-bromohexadecanamido)phenyl)-2,3,9-trimethyl-6H-thieno[3,2-f][1,2,4]triazolo[4,3-a][1,4]diazepin-6-yl)acetate (480.00 mg, 0.64 mmol, 1.00 equiv) and tryptamine (101.88 mg, 0.64 mmol, 1.00 equiv) in ACN (3.00 mL), K₂CO₃ (527.29 mg, 3.82 mmol, 6.00 equiv) was added and the resulting mixture was stirred for 17.0 h at 70 degrees C. The resulting mixture was filtered, the filter cake was washed with CH₃CN (3×5 mL). The filtrate was concentrated under reduced pressure and purified by reverse flash chromatography with the following conditions: column, C₁₈ silica gel; mobile phase, 0.05% NH₄HCO₃ in water and CH₃CN, 50% to 60% gradient in 15 min; detector, UV 254 and 220 nm. The fractions were combined and concentrated to afford tert-butyl (S)-2-(4-(4-(16-((2-(1H-indol-3-yl)ethyl)amino)hexadecanamido)phenyl)-2,3,9-trimethyl-6H-thieno[3,2-f][1,2,4]triazolo[4,3-a][1,4]diazepin-6-yl)acetate (100.00 mg, 15.08% yield) as white solid. LC/MS: mass calcd. For C₄₉H₆₇N₇O₃S: 833.50, found: 834.75 [M+H]⁺.

### Step 5: Synthesis of tert-butyl (S)-2-(4-(4-(16-((2-(1H-indol-3-yl)ethyl)(((9H-fluoren-9-yl)methoxy)carbonyl)amino)hexadecanamido)phenyl)-2,3,9-trimethyl-6H-thieno[3,2-f][1,2,4]triazolo[4,3-a][1,4]diazepin-6-yl)acetate

To a stirred solution of tert-butyl (S)-2-(4-(4-(16-((2-(1H-indol-3-yl)ethyl)amino)hexadecanamido)phenyl)-2,3,9-trimethyl-6H-thieno[3,2-f][1,2,4]triazolo[4,3-a][1,4]diazepin-6-yl)acetate (90.00 mg, 0.11 mmol, 1.00 equiv) in THF (2.00 mL), DIEA (41.83 mg, 0.32 mmol, 3.00 equiv) and 2,5-dioxopyrrolidin-1-yl 9H-fluoren-9-ylmethyl carbonate (36.39 mg, 0.11 mmol, 1.00 equiv) were added and the mixture was stirred for 4.0 h at room temperature. The resulting mixture was concentrated under vacuum and the residue was purified by Prep-TLC (DCM: MeOH=10:1) to afford tert-butyl (S)-2-(4-(4-(16-((2-(1H-indol-3-yl)ethyl)(((9H-fluoren-9-yl)methoxy)carbonyl)amino)hexadecanamido)phenyl)-2,3,9-trimethyl-6H-thieno[3,2-f][1,2,4]triazolo[4,3-a][1,4]diazepin-6-yl)acetate (80.00 mg, 56.15% yield) as white solid. LC/MS: mass calcd. For C₆₄H₇₇N₇O₅S: 1055.57, found: 1056.50 [M+H]⁺.

### Step 6: Synthesis of (S)-2-(4-(4-(16-((2-(1H-indol-3-yl)ethyl)(((9H-fluoren-9-yl) methoxy)carbonyl)amino)hexadecanamido)phenyl)-2,3,9-trimethyl-6H-thieno[3,2-f][1,2,4]triazolo[4,3-a][1,4]diazepin-6-yl)acetic Acid

The procedure was the same as methyl 4-[4-(4-amino-1-methylpyrrole-2-amido)-1-methylimidazole-2-amido]-1-methylpyrrole-2-carboxylate (Example 2 Step 4). 70.00 mg of tert-butyl (S)-2-(4-(4-(16-((2-(1H-indol-3-yl)ethyl)(((9H-fluoren-9-yl)methoxy)carbonyl)amino)hexadecanamido)phenyl)-2,3,9-trimethyl-6H-thieno[3,2-f][1,2,4]triazolo[4,3-a][1,4]diazepin-6-yl)acetate was used, 70.00 mg crude of desired product was obtained as light yellow oil. LC/MS: mass calcd. For C₆₀H₆₉N₇O₅S: 999.50, found: 1000.80 [M+H]⁺.

### Example 17. Synthesis of (S)-2-(2,3,9-trimethyl-4-(4-octanamidophenyl)-6H-thieno[3,2-f][1,2,4]triazolo[4,3-a][1,4]diazepin-6-yl]acetic Acid

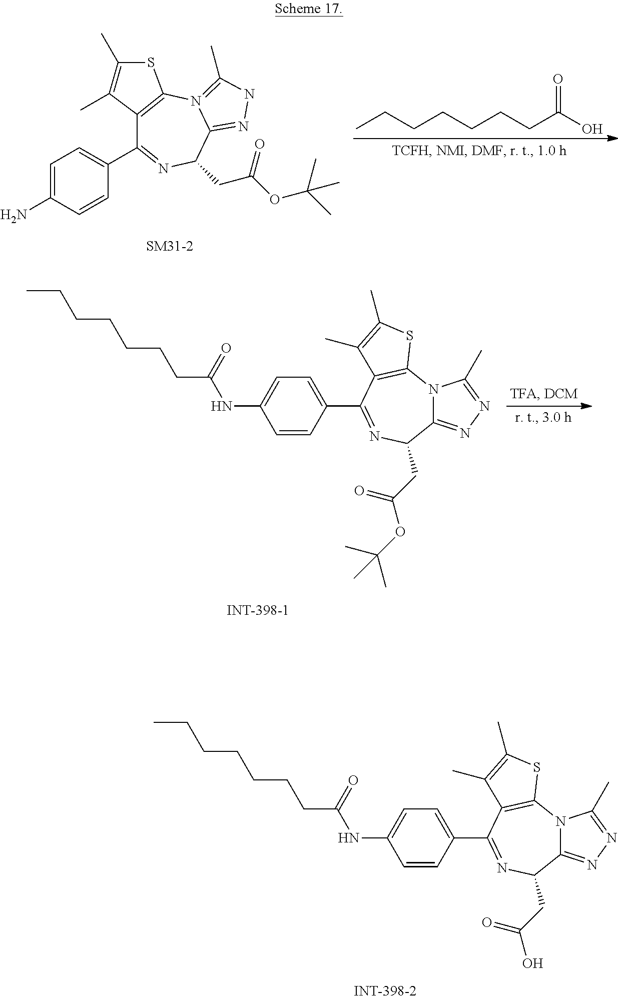

### Step 1: Synthesis of tert-butyl (S)-2-(2,3,9-trimethyl-4-(4-octanamidophenyl)-6H-thieno[3,2-f][1,2,4]triazolo[4,3-a][1,4]diazepin-6-yl)acetate

The procedure was the same as tert-butyl (S)-2-(4-(4-(16-bromohexadecanamido)phenyl)-2,3,9-trimethyl-6H-thieno[3,2-f][1,2,4]triazolo[4,3-a][1,4]diazepin-6-yl)acetate. 100.00 mg of tert-butyl (S)-2-(4-(4-aminophenyl)-2,3,9-trimethyl-6H-thieno[3,2-f][1,2,4]triazolo[4,3-a][1,4]diazepin-6-yl)acetate was used, 117.00 mg crude of desired product was obtained as yellow solid. LC/MS: mass calcd. For C₃₁H₄₁N₅O₃S: 563.29, found: 564.45 [M+H]⁺.

### Step 2: Synthesis of (S)-2-(2,3,9-trimethyl-4-(4-octanamidophenyl)-6H-thieno[3,2-f][1,2,4]triazolo[4,3-a][1,4]diazepin-6-yl)acetic Acid

The procedure was the same as methyl 4-[4-(4-amino-1-methylpyrrole-2-amido)-1-methylimidazole-2-amido]-1-methylpyrrole-2-carboxylate (Example 2 Step 4). 110.00 mg of tert-butyl (S)-2-(2,3,9-trimethyl-4-(4-octanamidophenyl)-6H-thieno[3,2-f][1,2,4]triazolo[4,3-a][1,4]diazepin-6-yl)acetate was used, 110.00 mg crude of desired product was obtained as yellow oil. LC/MS: mass calcd. For C₂₇H₃₃N₅O₃S: 507.23, found: 508.35 [M+H]⁺.

### Example 18. Synthesis of (S)-2-(2,3,9-trimethyl-4-(4-palmitamidophenyl)-6H-thieno[3,2-f][1,2,4]triazolo[4,3-a][1,4]diazepin-6-yl]acetic Acid

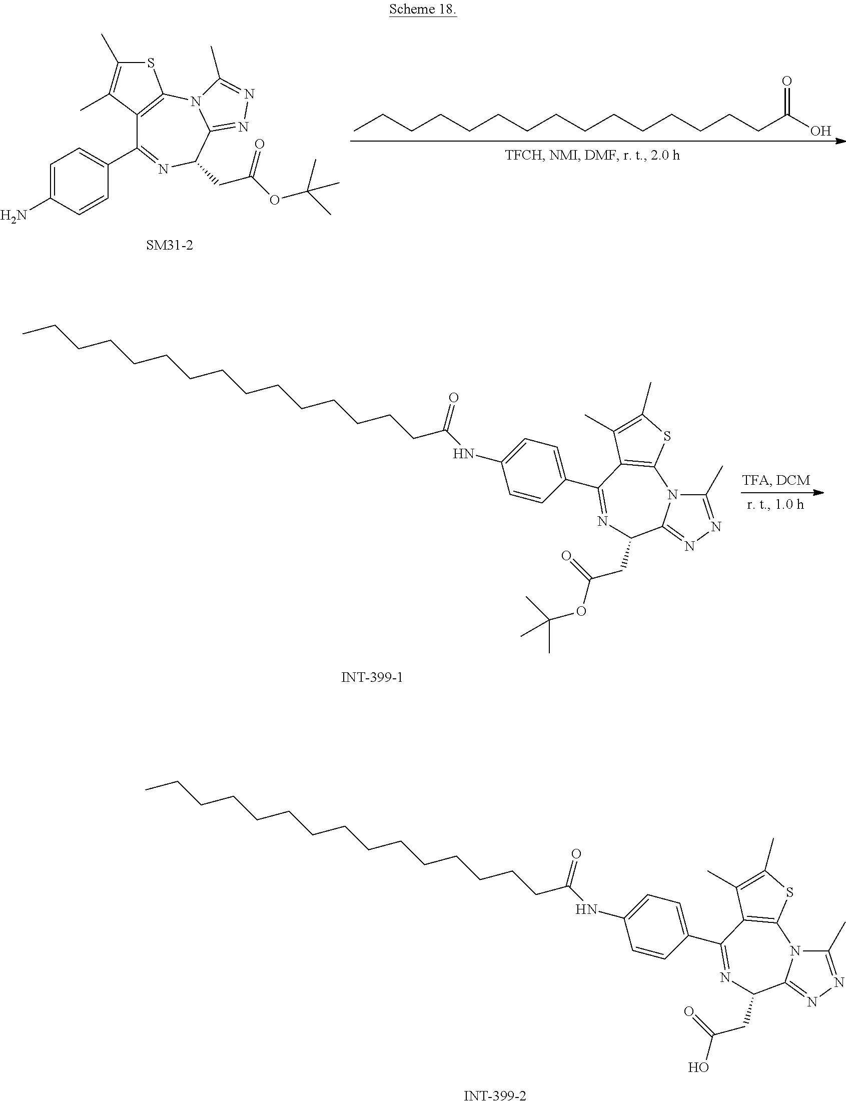

### Step 1: Synthesis of tert-butyl (S)-2-(2,3,9-trimethyl-4-(4-palmitamidophenyl)-6H-thieno[3,2-f][1,2,4]triazolo[4,3-a][1,4]diazepin-6-yl)acetate

The procedure was the same as ethyl 4-(1-methylimidazole-2-amido)-1-[26-(2,5,8,11,14,17,20,23-octaoxapentacosan-25-yl)-2,5,8,11,14,17,20,23-octaoxa-26-azahentriacontan-31-yl]pyrrole-2-carboxylate, but the reaction time was 2.0 h. 60.00 mg of palmitic acid was used, 120.00 mg of desired product was obtained as yellow oil (75.87% yield). LC/MS: mass calcd. For C₃₉H₅₇N₅O₃S: 675.42, found: 676.65 [M+H]⁺.

### Step 2: Synthesis of (S)-2-(2,3,9-trimethyl-4-(4-palmitamidophenyl)-6H-thieno[3,2-f][1,2,4]triazolo[4,3-a][1,4]diazepin-6-yl)acetic Acid

The procedure was the same as methyl 4-[4-(4-amino-1-methylpyrrole-2-amido)-1-methylimidazole-2-amido]-1-methylpyrrole-2-carboxylate (Example 2 Step 4). 110.00 mg of tert-butyl (S)-2-(2,3,9-trimethyl-4-(4-palmitamidophenyl)-6H-thieno[3,2-f][1,2,4]triazolo[4,3-a][1,4]diazepin-6-yl)acetate was used, 110.00 mg crude of desired product was obtained as yellow oil. LC/MS: mass calcd. For C₃₅H₄₉N₅O₃S: 619.36, found: 620.50 [M+H]⁺.

### Example 19. Synthesis of 4-[2-(2,4-difluorophenoxy)-5-methanesulfonylphenyl]-6-methyl-7-oxo-1H-pyrrolo[2,3-c]pyridine-2-carboxylic Acid

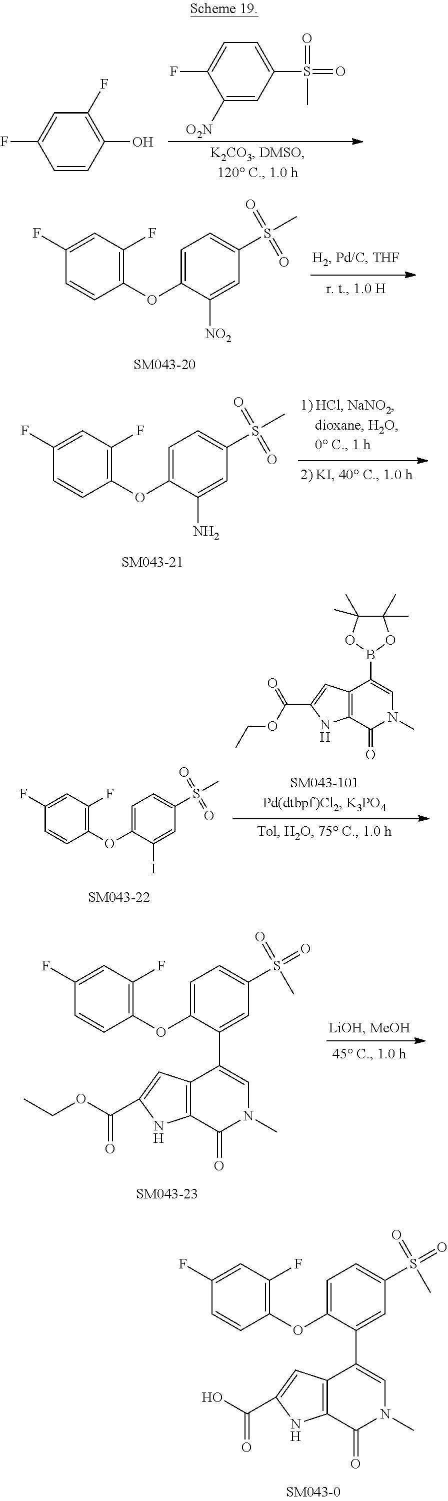

### Step 2: Synthesis of 2-(2,4-difluorophenoxy)-5-methanesulfonylaniline

The procedure was the same as 2-(2,4-difluorophenoxy)-5-methanesulfonylaniline (INT-444-2), but the reaction time was 1.0 h. 500.00 mg of 1-(2,4-difluorophenoxy)-4-methanesulfonyl-2-nitrobenzene was used, 420.00 mg of 2-(2,4-difluorophenoxy)-5-methanesulfonylaniline was obtained as colorless oil.

LC/MS: mass calcd. For C₁₃H₁₁F₂NO₃S: 299.04, found: 300.05 [M+H]⁺.

### Step 3: Synthesis of 1-(2,4-difluorophenoxy)-2-iodo-4-methanesulfonylbenzene

The procedure was the same as 5-fluoro-2-(2-iodo-4-methanesulfonylphenoxy)-1,3-dimethylbenzene (Example 13 Step 5), but the reaction time was 1.0 h after KI was added. 420.00 mg of 5-fluoro-2-(2-iodo-4-methanesulfonylphenoxy)-1,3-dimethylbenzene was used, 440.00 mg of 1-(2,4-difluorophenoxy)-2-iodo-4-methanesulfonylbenzene was obtained as yellow solid (78.57% yield). ¹H NMR (400 MHz, DMSO-d₆) δ: 8.38 (s, 1H), 7.86 d, J=8.4 Hz, 1H), 7.50-7.61 (m, 1H), 7.41-7.49 (m, 1H), 7.15-7.25 (m, 1H), 6.90 (d, J=8.8 Hz, 1H), 3.26 (s, 3H).

### Step 4: Synthesis of ethyl 4-[2-(2,4-difluorophenoxy)-5-methanesulfonylphenyl]-6-methyl-7-oxo-1H-pyrrolo[2,3-c]pyridine-2-carboxylate

The procedure was the same as ethyl 4-[2-(4-fluoro-2,6-dimethylphenoxy)-5-methanesulfonylphenyl]-6-methyl-7-oxo-1H-pyrrolo[2,3-c]pyridine-2-carboxylate (INT-444-4), but the reaction temperature was 75° C. and reaction time was 1.0 h. 420.00 mg of 1-(2,4-difluorophenoxy)-2-iodo-4-methanesulfonylbenzene was used, 340.00 mg of ethyl 4-[2-(2,4-difluorophenoxy)-5-methanesulfonylphenyl]-6-methyl-7-oxo-1H-pyrrolo[2,3-c]pyridine-2-carboxylate was obtained as white solid (62.11% yield). LC/MS: mass calcd. For C₂₄H₂₀F₂N₂O₆S: 502.10, found: 503.25 [M+H]⁺.

### Step 5: Synthesis of 4-[2-(2,4-difluorophenoxy)-5-methanesulfonylphenyl]-6-methyl-7-oxo-1H-pyrrolo[2,3-c]pyridine-2-carboxylic Acid

The procedure was the same as 4-[3-[(tert-butoxycarbonyl)amino] propanamido]-1-methylimidazole-2-carboxylic acid (Example 1 Step 3), but the reaction time was 1.0 h. 320.00 mg of ethyl 4-[2-(2,4-difluorophenoxy)-5-methanesulfonylphenyl]-6-methyl-7-oxo-1H-pyrrolo[2,3-c]pyridine-2-carboxylate was used, 290.00 mg of 4-[2-(2,4-difluorophenoxy)-5-methanesulfonylphenyl]-6-methyl-7-oxo-1H-pyrrolo[2,3-c]pyridine-2-carboxylic acid was obtained as white solid (92.15% yield). LC/MS: mass calcd. For C₂₂H₁₆F₂N₂O₆S: 474.07, found: 475.20 [M+H]⁺.

### Synthesis of Representative Compounds of the Disclosure

### Example 20. Synthesis of N-(5-{[2-({2-[(2-{[26-(4-{1-ethyl-4-[2-(4-fluoro-2,6-dimethylphenoxy)-5-(2-hydroxypropan-2-yl)phenyl]-6-methyl-7-oxopyrrolo[2,3-c]pyridine-2-amido}phenoxy)-3,6,9,12,15,18,21,24-octaoxahexacosan-1-yl]carbamoyl]ethyl)carbamoyl]-1-methylimidazol-4-yl]carbamoyl)ethyl]carbamoyl]-1-methylpyrrol-3-yl)-1-methyl-4-(3-{[1-methyl-4-(1-methylimidazole-2-amido)pyrrol-2-yl]formamido]propanamido)imidazole-2-carboxamid (Compound 196)

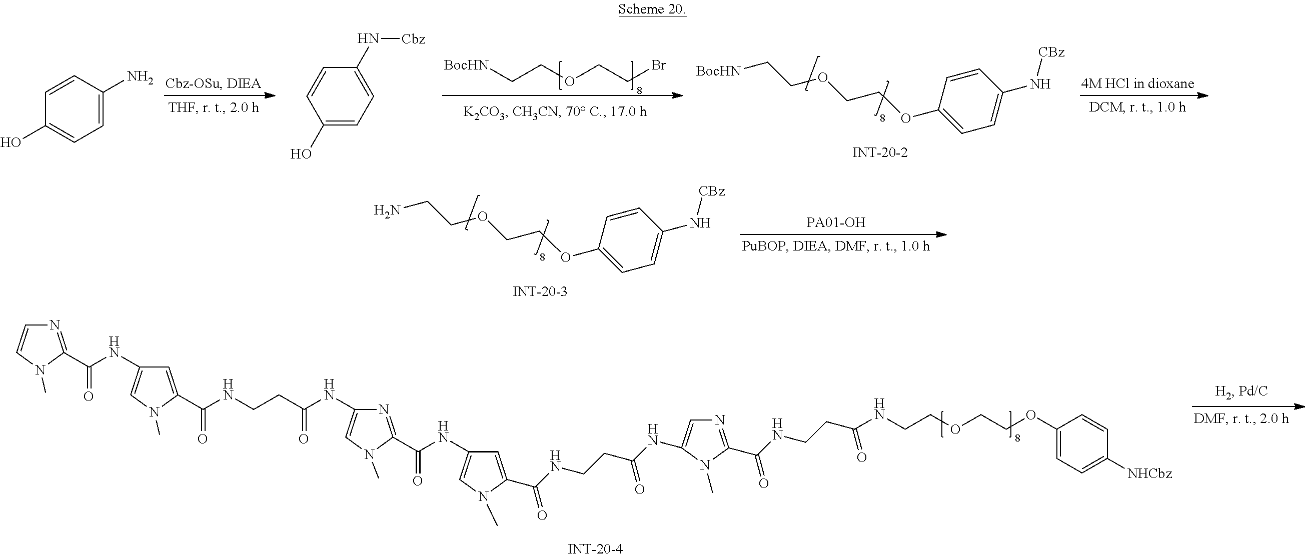

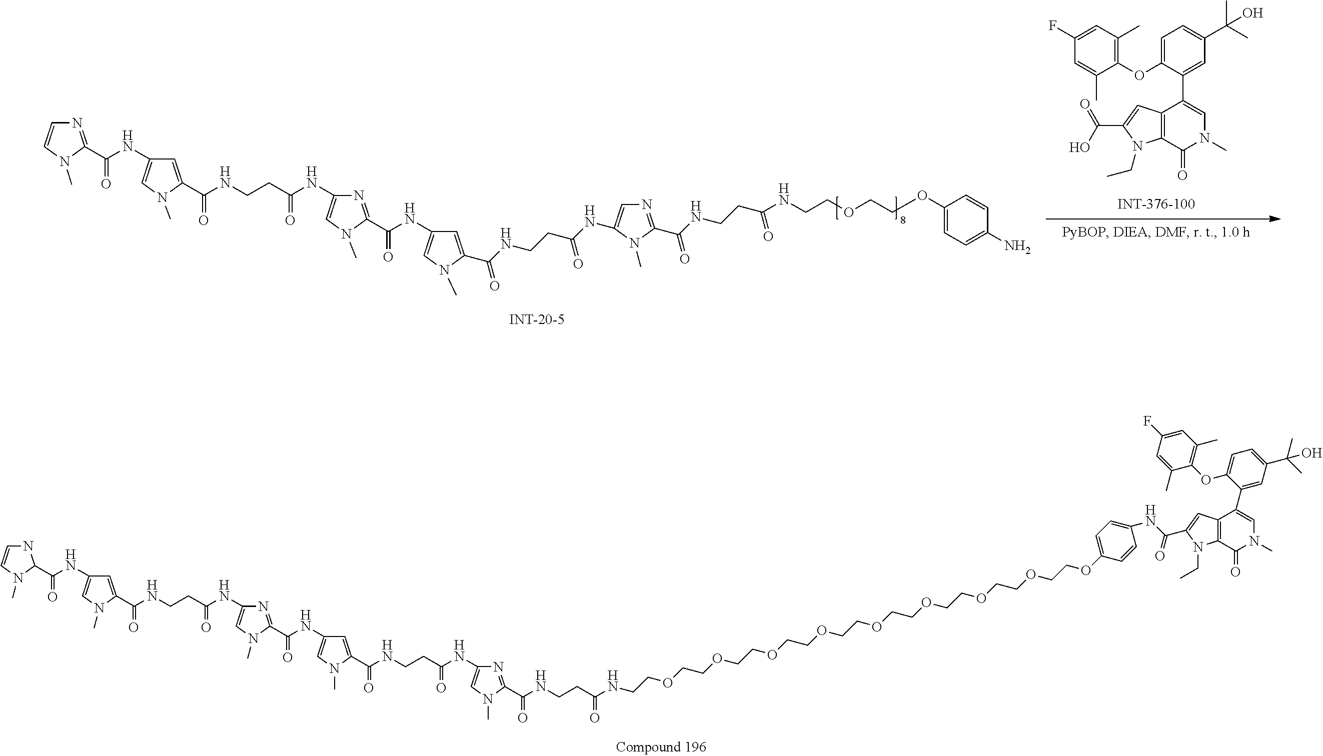

### Step 1: Synthesis of benzyl N-(4-hydroxyphenyl)carbamate

A solution of aminophenol (10.00 g, 91.64 mmol, 1.00 equiv) and benzyl 2,5-dioxopyrrolidin-1-yl carbonate (27.00 g, 108.34 mmol, 1.18 equiv), DIEA (29.02 g, 224.51 mmol, 2.45 equiv) in THF (60.00 mL) was stirred for 2.0 h at room temperature. The resulting mixture was extracted with EtOAc (3×150 mL). The combined organic layers were washed with brine (3×50 mL), dried over anhydrous Na₂SO₄. After filtration, the filtrate was concentrated under reduced pressure. The resulting pink solid was washed with 3×30 mL of ACN. This resulted in benzyl N-(4-hydroxyphenyl)carbamate (15.00 g, 63.93% yield) as white solid. LC/MS: mass calcd. For C₁₄H₁₃NO₃: 243.09, found: 244.10 [M+H]⁺.

### Step 2: Synthesis of benzyl N-[4-({26-[(tert-butoxycarbonyl) amino]-3,6,9,12,15,18,21,24-octaoxahexacosan-1-yl}oxy)phenyl]carbamate

The procedure was the same as ethyl 1-(5-bromopentyl)-4-[(tert-butoxycarbonyl)amino]pyrrole-2-carboxylate, but the reaction time was 17.0 h. 2.80 g of benzyl N-(4-hydroxyphenyl)carbamate was used, 7.00 g of benzyl N-[4-({26-[(tert-butoxycarbonyl)amino]-3,6,9,12,15,18,21,24-octaoxahexacosan-1-yl}oxy)phenyl]carbamate was obtained as colorless oil (82.31% yield). LC/MS: mass calcd. For C₃₇H₅₈N₂O₁₃: 738.39, found: 756.55 [M+H₂O]+.

### Step 3: Synthesis of benzyl N-{4-[(26-amino-3,6,9,12,15,18,21,24-octaoxahexacosan-1-yl)oxy]phenyl}carbamate

The procedure was the same as methyl 4-[4-(3-aminopropanamido)-1-methylimidazole-2-amido]-1-methylpyrrole-2-carboxylate hydrochloride, but the reaction solvent was 4M HCl in dioxane/DCM (1:1). 6.50 g of benzyl N-[4-({26-[(tert-butoxycarbonyl)amino]-3,6,9,12,15,18,21,24-octaoxahexacosan-1-yl}oxy)phenyl]carbamate was used, 6.50 g crude of benzyl N-{4-[(26-amino-3,6,9,12,15,18,21,24-octaoxahexacosan-1-yl)oxy]phenyl}carbamate was obtained as colorless oil. LC/MS: mass calcd. For C₃₂H₅₈N₂O₁₃: 638.34, found: 639.40 [M+H]⁺.

### Step 4: Synthesis of benzyl N-[4-({26-[3-({1-methyl-4-[3-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)propanamido]imidazol-2-yl}formamido)propanamido]-3,6,9,12,15,18,21,24-octaoxahexacosan-1-yl}oxy)phenyl]carbamate

The procedure was the same as methyl 4-(4-{4-[(tert-butoxycarbonyl)amino]-1-methylpyrrole-2-amido}-1-methylimidazole-2-amido)-1-methylpyrrole-2-carboxylate (Example 2 Step 3). 9.00 g of benzyl N-{4-[(26-amino-3,6,9,12,15,18,21,24-octaoxahexacosan-1-yl)oxy]phenyl}carbamate was used, 15.50 g of benzyl N-[4-({26-[3-({1-methyl-4-[3-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)propanamido]imidazol-2-yl}formamido)propanamido]-3,6,9,12,15,18,21,24-octaoxahexacosan-1-yl}oxy)phenyl]carbamate was obtained as yellow solid (75.84% yield). LC/MS: mass calcd. For C₆₈H₉₁N₁₇O₁₉: 1449.67, found: 1450.90 [M+H]⁺.

### Step 5: Synthesis of N-(5-{[2-({2-[(2-{[26-(4-aminophenoxy)-3,6,9,12,15,18,21,24-octaoxahexacosan-1-yl]carbamoyl}ethyl)carbamoyl]-1-methylimidazol-4-yl}carbamoyl)ethyl]carbamoyl}-1-methylpyrrol-3-yl)-1-methyl-4-(3-{[1-methyl-4-(1-methylimidazole-2-amido)pyrrol-2-yl]formamido}propanamido)imidazole-2-carboxamide

To a solution of benzyl N-[4-({26-[3-({1-methyl-4-[3-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido) propanamido]imidazol-2-yl}formamido)propanamido]-3,6,9,12,15,18,21,24-octaoxahexacosan-1-yl}oxy) phenyl]carbamate (5.50 g, 3.792 mmol, 1.00 equiv) in DMF (40.00 mL) was added Pd/C (1.10 g, 20% w/w). Then H₂ was exchanged by three times. The mixture was stirred at room temperature for 2.0 h under H₂ atmosphere. The Pd/C was filtered out and washed by MeOH, the filtration was concentrated and lyophilized. The resulting mixture was poured into Water/Ice(60 mL). The precipitated solids were collected by filtration, washed with H₂O (3×20 mL) and lyophilized. N-(5-{[2-({2-[(2-{[26-(4-aminophenoxy)-3,6,9,12,15,18,21,24-octaoxahexacosan-1-yl]carbamoyl}ethyl)carbamoyl]-1-methylimidazol-4-yl}carbamoyl)ethyl]carbamoyl}-1-methylpyrrol-3-yl)-1-methyl-4-(3-{[1-methyl-4-(1-methylimidazole-2-amido)pyrrol-2-yl]formamido}propanamido)imidazole-2-carboxamide (4.08 g, 81.74%) was obtained as white solid. LC/MS: mass calcd. For C₆₀H₈₅N₁₇O: 1315.63, found: 1317.10 [M+H]⁺.

### Step 6: Synthesis of Compound 196

Into a 25 mL flask was added 1-ethyl-4-[2-(4-fluoro-2,6-dimethylphenoxy)-5-(2-hydroxypropan-2-yl)phenyl]-6-methyl-7-oxopyrrolo[2,3-c]pyridine-2-carboxylic acid (280.00 mg, 0.57 mmol, 1.00 equiv) and DMF (3.00 mL). The mixture was cooled to 0 degrees C., then PyBOP (443.74 mg, 0.85 mmol, 1.50 equiv) and N-(5-{[2-({2-[(2-{[26-(4-aminophenoxy)-3,6,9,12,15,18,21,24-octaoxahexacosan-1-yl]carbamoyl}ethyl)carbamoyl]-1-methylimidazol-4-yl}carbamoyl)ethyl]carbamoyl}-1-methylpyrrol-3-yl)-1-methyl-4-(3-{[1-methyl-4-(1-methylimidazole-2-amido)pyrrol-2-yl]formamido}propanamido)imidazole-2-carboxamide (748.36 mg, 0.57 mmol, 1.00 equiv) were added followed by addition of DIEA (220.41 mg, 1.70 mmol, 3.00 equiv) in portions. The reaction was stirred at room temperature for 1.0 h. The reaction mixture was filtered and the filtration in DMF (3.00 mL) was purified by Prep-HPLC with the following conditions: Column: XSelect CSH Prep C18 OBD Column, 19*250 mm, 5 μm; Mobile Phase A: Water(10 mmol/L NH₄HCO₃+0.1% NH₃·H₂O), Mobile Phase B: ACN; Flow rate: 25 mL/min; Gradient: 48% B to 56% B in 10 min, 56% B; Wave Length: 254 nm; RT1(min): 9.02; Number Of Runs: 0. The fractions were combined and lyophilized directly. This result in N-(5-{[2-({2-[(2-{[26-(4-{1-ethyl-4-[2-(4-fluoro-2,6-dimethylphenoxy)-5-(2-hydroxypropan-2-yl)phenyl]-6-methyl-7-oxopyrrolo[2,3-c]pyridine-2-amido}phenoxy)-3,6,9,12,15,18,21,24-octaoxahexacosan-1-yl]carbamoyl}ethyl)carbamoyl]-1-methylimidazol-4-yl}carbamoyl)ethyl]carbamoyl}-1-methylpyrrol-3-yl)-1-methyl-4-(3-{[1-methyl-4-(1-methylimidazole-2-amido)pyrrol-2-yl]formamido}propanamido)imidazole-2-carboxamide (171.10 mg, 16.53% yield) as white solid. HRMS: mass calcd. For C88H₁₁₂FN₁₉O₂₁: 1789.8264, found: 1790.8420 [M+H]⁺.

Examples 21-27 were made by the procedures of Example 20.

### Example 21. Synthesis of N-(5-{[2-({2-[(2-{[26-(4-{14-[2-(4-fluoro-2,6-dimethylphenoxy)-5-(prop-1-en-2-yl)phenyl]-6-methyl-7-oxo-1H-pyrrolo[2,3-c]pyridine-2-amido}phenoxy)-3,6,9,12,15,18,21,24-octaoxahexacosan-1-yl]carbamoyl]ethyl)carbamoyl]-1-methylimidazol-4-yl]carbamoyl)ethyl]carbamoyl]-1-methylpyrrol-3-yl)-1-methyl-4-(3-{[1-methyl-4-(1-methylimidazole-2-amido)pyrrol-2-yl]formamido]propanamido)imidazole-2-carboxamide (Compound 197)

300.00 mg of N-(5-{[2-({2-[(2-{[26-(4-aminophenoxy)-3,6,9,12,15,18,21,24-octaoxahexacosan-1-yl]carbamoyl}ethyl)carbamoyl]-1-methylimidazol-4-yl}carbamoyl)ethyl]carbamoyl}-1-methylpyrrol-3-yl)-1-methyl-4-(3-{[1-methyl-4-(1-methylimidazole-2-amido)pyrrol-2-yl]formamido}propanamido)imidazole-2-carboxamide was used, 232.40 mg of desired product was obtained as white solid (56.28% yield). HRMS: mass calcd. for C86H₁₀₆FN₁₉O₂₀: 1743.7845, found: 1744.7899 [M+H]⁺.

### Example 22. Synthesis of 1-methyl-4-(3-{[1-methyl-4-(1-methylimidazole-2-amido)pyrrol-2-yl]formamido}propanamido)-N-(1-methyl-5-{[2-({1-methyl-2-[(2-{[26-(4-{6-methyl-7-oxo-1H-pyrrolo[2,3-c]pyridine-2-amido}phenoxy)-3,6,9,12,15,18,21,24-octaoxahexacosan-1-yl]carbamoyl]ethyl)carbamoyl]imidazol-4-yl]carbamoyl)ethyl]carbamoyl]pyrrol-3-yl)imidazole-2-carboxamide (Compound 198)

400.00 mg of N-(5-{[2-({2-[(2-{[26-(4-aminophenoxy)-3,6,9,12,15,18,21,24-octaoxahexacosan-1-yl]carbamoyl}ethyl)carbamoyl]-1-methylimidazol-4-yl}carbamoyl)ethyl]carbamoyl}-1-methylpyrrol-3-yl)-1-methyl-4-(3-{[1-methyl-4-(1-methylimidazole-2-amido)pyrrol-2-yl]formamido}propanamido)imidazole-2-carboxamide was used, 242.00 mg of desired product was obtained as white solid (51.22% yield). HRMS: mass calcd. for C69H₉₁N₁₉O₁₉: 1489.6738, found: 1490.6778 [M+H]⁺.

### Example 23. Synthesis of (S)-1-methyl-4-(3-(1-methyl-4-(1-methyl-1H-imidazole-2-carboxamido)-1H-pyrrole-2-carboxamido)propanamido)-N-(1-methyl-5-((3-((1-methyl-2-((28-oxo-1-(4-(2-(2,3,9-trimethyl-4-(4-octanamidophenyl)-6H-thieno[3,2-f][1,2,4]triazolo[4,3-a][1,4]diazepin-6-yl)acetamido)phenoxy)-3,6,9,12,15,18,21,24-octaoxa-27-azatriacontan-30-yl)carbamoyl)-1H-imidazol-4-yl)amino)-3-oxopropyl)carbamoyl)-1H-pyrrol-3-yl)-1H-imidazole-2-carboxamide (Compound 203)

50.00 mg of (S)-2-(2,3,9-trimethyl-4-(4-octanamidophenyl)-6H-thieno[3,2-f][1,2,4]triazolo[4,3-a][1,4]diazepin-6-yl)acetic acid was used, 39.70 mg of desired product was obtained as white solid (21.94% yield). HRMS: mass calcd. for C₈₇H₁₁₆N₂₂O₁₉S Exact Mass: 1804.8508, found: 1805.8650 [M+H]⁺.

### Example 24. Synthesis of (S)-1-methyl-4-(3-(1-methyl-4-(1-methyl-1H-imidazole-2-carboxamido)-1H-pyrrole-2-carboxamido)propanamido)-N-(1-methyl-5-((3-((1-methyl-2-((28-oxo-1-(4-(2-(2,3,9-trimethyl-4-(4-palmitamidophenyl)-6H-thieno[3,2-f][1,2,4]triazolo[4,3-a][1,4]diazepin-6-yl)acetamido)phenoxy)-3,6,9,12,15,18,21,24-octaoxa-27-azatriacontan-30-yl)carbamoyl)-1H-imidazol-4-yl)amino)-3-oxopropyl)carbamoyl)-1H-pyrrol-3-yl)-1H-imidazole-2-carboxamide (Compound 204)

110.00 mg of (S)-2-(2,3,9-trimethyl-4-(4-palmitamidophenyl)-6H-thieno[3,2-f][1,2,4]triazolo[4,3-a][1,4]diazepin-6-yl)acetic acid was used, 61.10 mg of desired product was obtained as white solid (17.25% yield). HRMS: mass calcd. for C95H₁₃₂N₂₂O₁₉S: 1916.9760, found: 1917.9832 [M+H]⁺.

### Example 25. Synthesis of N-(5-{[2-({2-[(2-{[26-(4-{14-[2-(2,4-difluorophenoxy)-5-methanesulfonylphenyl]-6-methyl-7-oxo-1H-pyrrolo[2,3-c]pyridine-2-amido}phenoxy)-3,6,9,12,15,18,21,24-octaoxahexacosan-1-yl]carbamoyl]ethyl)carbamoyl]-1-methylimidazol-4-yl}carbamoyl)ethyl]carbamoyl}-1-methylpyrrol-3-yl)-1-methyl-4-(3-{[1-methyl-4-(1-methylimidazole-2-amido)pyrrol-2-yl]formamido]propanamido)imidazole-2-carboxamide (Compound 207)

150.00 mg of N-(5-{[2-({2-[(2-{[26-(4-aminophenoxy)-3,6,9,12,15,18,21,24-octaoxahexacosan-1-yl]carbamoyl}ethyl)carbamoyl]-1-methylimidazol-4-yl}carbamoyl)ethyl]carbamoyl}-1-methylpyrrol-3-yl)-1-methyl-4-(3-{[1-methyl-4-(1-methylimidazole-2-amido)pyrrol-2-yl]formamido}propanamido)imidazole-2-carboxamide was used, 42.30 mg of desired product was obtained as white solid (20.40% yield). HRMS: mass calcd. for C82H₉₉F₂N₁₉O₂₂S Exact Mass: 1771.6900, found: 1772.6914 [M+H]⁺.

### Example 26. Synthesis of (R)—N-(5-((3-((2-((1-(4-(2-(4-(4-chlorophenyl)-2,3,9-trimethyl-6H-thieno[3,2-f[1,2,4]triazolo[4,3-a][1,4]diazepin-6-yl)acetamido)phenoxy)-28-oxo-3,6,9,12,15,18,21,24-octaoxa-27-azatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide (Compound 231)

87.40 mg of N-(5-{[2-({2-[(2-{[26-(4-aminophenoxy)-3,6,9,12,15,18,21,24-octaoxahexacosan-1-yl]carbamoyl}ethyl)carbamoyl]-1-methylimidazol-4-yl}carbamoyl)ethyl]carbamoyl}-1-methylpyrrol-3-yl)-1-methyl-4-(3-{[1-methyl-4-(1-methylimidazole-2-amido)pyrrol-2-yl]formamido}propanamido)imidazole-2-carboxamide was used, 43.70 mg of desired product was obtained as white solid (38.51% yield). HRMS: mass calcd. for C79H₁₀₀ClN₂₁O₁₈: 1697.6964, found: 1698.7020 [M+H]⁺.

### Example 27. Synthesis of (S)—N-(5-((3-((2-((1-(4-(2-(4-(4-(16-((2-(1H-indol-3-yl)ethyl)amino)hexadecanamido)phenyl)-2,3,9-trimethyl-6H-thieno[3,2-f[1,2,4]triazolo[4,3-a][1,4]diazepin-6-yl)acetamido)phenoxy)-28-oxo-3,6,9,12,15,18,21,24-octaoxa-27-azatriacontan-30-yl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide (Compound 200)

To a stirred solution of (S)-2-(4-(4-(16-((2-(1H-indol-3-yl)ethyl)(((9H-fluoren-9-yl)methoxy)carbonyl)amino)hexadecanamido)phenyl)-2,3,9-trimethyl-6H-thieno[3,2-f][1,2,4]triazolo[4,3-a][1,4]diazepin-6-yl)acetic acid (60.00 mg, 0.06 mmol, 1.00 equiv) in DMF (2.00 mL), PyBOP (31.21 mg, 0.06 mmol, 1.00 equiv), N-(5-{[2-({2-[(2-{[26-(4-aminophenoxy)-3,6,9,12,15,18,21,24-octaoxahexacosan-1-yl]carbamoyl}ethyl)carbamoyl]-1-methylimidazol-4-yl}carbamoyl)ethyl]carbamoyl}-1-methylpyrrol-3-yl)-1-methyl-4-(3-{[1-methyl-4-(1-methylimidazole-2-amido)pyrrol-2-yl]formamido}propanamido)imidazole-2-carboxamide (78.96 mg, 0.06 mmol, 1.00 equiv) and DIEA (23.26 mg, 0.180 mmol, 3.00 equiv) were added in turn to the solution at 0 degrees C. The mixture was allowed to warm to room temperature and stirred for 1.0 h. Then piperidine (0.20 mL) (the volume ratio of Piperidine and DMF=1:10) was added and the mixture was stirred for 1.0 h at room temperature. After the reaction was completed, the mixture was added to the ice water (5 mL) dropwise. The solid was generated, filtered out, washed by water (2×3 mL), and dried under vacuum. The crude product (80.00 mg) was dissolved in DMF (2.00 mL), the resulting mixture was filtered and the filtration was purified by Prep-HPLC with the following conditions: Column: XBridge Shield RP18 OBD Column, 19*250 mm, 10 μm; Mobile Phase A: Water (0.05% TFA), Mobile Phase B: ACN; Flow rate: 25 mL/min; Gradient: 40% B to 53% B in 10 min, 53% B; Wave Length: 254 nm; RT1(min): 9.83; Number Of Runs: 0. The fractions were combined and lyophilized directly to afford desired product (15.00 mg, 20.13%) as white solid. HRMS: mass calcd. for C₁₀₅H₁₄₂N₂₄O₁₉S: 2075.0603, found: 2076.0676 [M+H]⁺.

### Example 28. Synthesis of N-(5-{[2-({2-[(2-{[26-(4-{4-[2-(4-fluoro-2,6-dimethylphenoxy)-5-(2-hydroxypropan-2-yl)phenyl]-6-methyl-7-oxo-1H-pyrrolo[2,3-c]pyridine-2-amido}phenoxy)-3,6,9,12,15,18,21,24-octaoxahexacosan-1-yl]carbamoyl]ethyl)carbamoyl]-1-methylimidazol-4-yl}carbamoyl)ethyl]carbamoyl]-1-[14-(2,5,8,11-tetraoxatridecan-13-yl)-2,5,8,11-tetraoxa-14-azanonadecan-19-yl]pyrrol-3-yl)-1-methyl-4-(3-{[1-methyl-4-(1-methylimidazole-2-amido)pyrrol-2-yl]formamido]propanamido)imidazole-2-carboxamide (Compound 241)

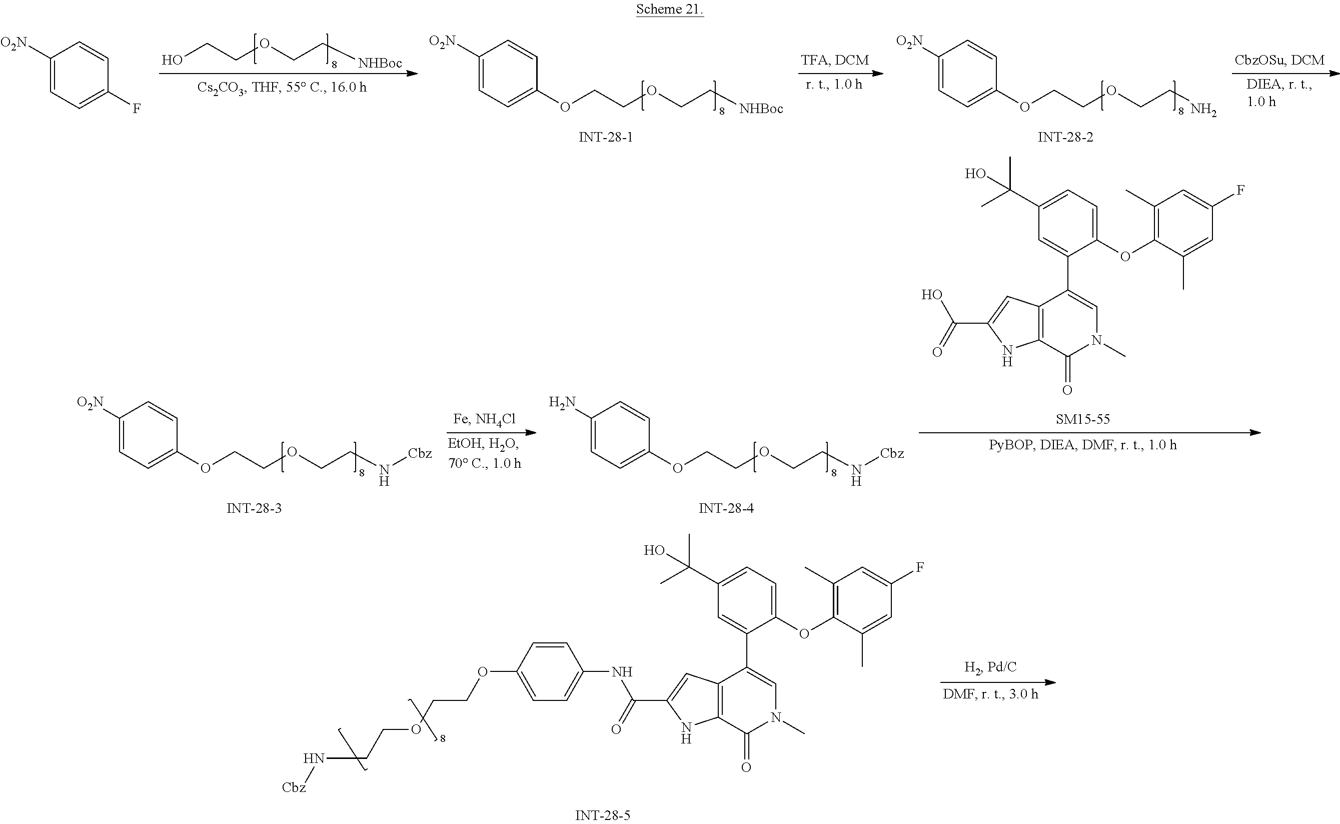

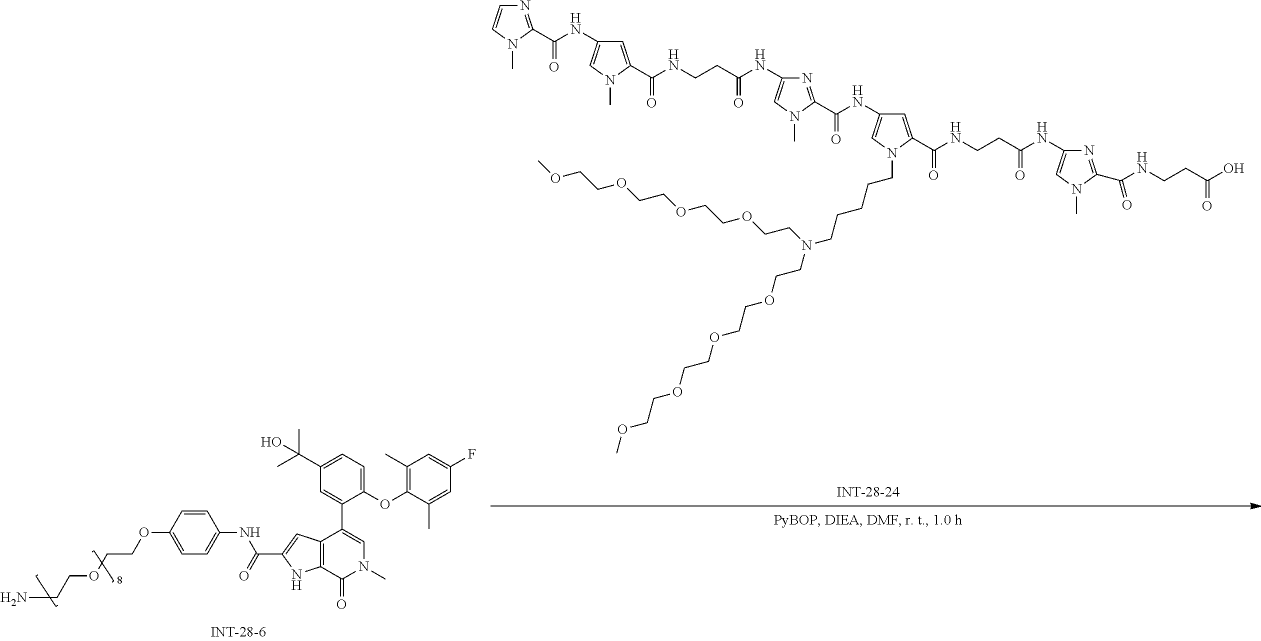

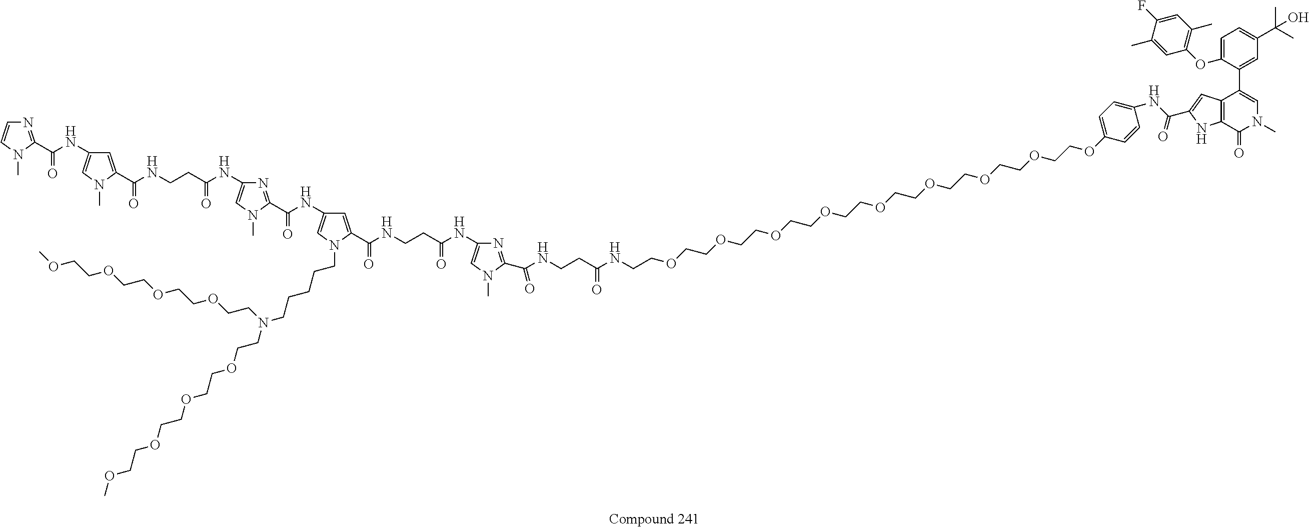 [SMILES: COCCOCCOCCOCCN(CCCCCn1cc(NC(=O)c2nc(NC(=O)CCNC(=O)c3cc(NC(=O)c4nccn4C)cn3C)cn2C)cc1C(=O)NCCC(=O)Nc1cn(C)c(C(=O)NCCC(=O)NCCOCCOCCOCCOCCOCCOCCOCCOCCOc2ccc(NC(=O)c3cc4c(-c5cc(C(C)(C)O)ccc5Oc5cc(C)c(F)cc5C)cn(C)c(=O)c4[nH]3)cc2)n1)CCOCCOCCOCCOC]

### Step 1: Synthesis of tert-butyl N-[26-(4-nitrophenoxy)-3,6,9,12,15,18,21,24-octaoxahexacosan-1-yl]carbamate

To a stirred solution of 4-fluoronitrobenzene (329.66 mg, 2.34 mmol, 1.20 equiv) in THF (15.00 mL) was added tert-butyl N-(26-hydroxy-3,6,9,12,15,18,21,24-octaoxahexacosan-1-yl)carbamate (1.00 g, 1.95 mmol, 1.00 equiv) and C₈₂CO₃ (1.90 g, 5.84 mmol, 3.00 equiv). The resulting mixture was stirred at 55 degrees C. for 16.0 h. The resulting mixture was concentrated under vacuum. The residue was purified by silica gel column chromatography (0-10% MeOH/DCM) to afford tert-butyl N-[26-(4-nitrophenoxy)-3,6,9,12,15,18,21,24-octaoxahexacosan-1-yl]carbamate (1.10 g, 89.01% yield) as yellow oil. LC/MS: mass calcd. For C29H₅₀N₂O₁₃: 634.33, found: 635.55 [M+H]⁺.

### Step 2: Synthesis of 26-(4-nitrophenoxy)-3,6,9,12,15,18,21,24-octaoxahexacosan-1-amine

The procedure was the same as methyl 4-[4-(4-amino-1-methylpyrrole-2-amido)-1-methylimidazole-2-amido]-1-methylpyrrole-2-carboxylate (Example 2 Step 4). 1.10 g of tert-butyl N-[26-(4-nitrophenoxy)-3,6,9,12,15,18,21,24-octaoxahexacosan-1-yl]carbamate was used, 920.0 mg crude of 26-(4-nitrophenoxy)-3,6,9,12,15,18,21,24-octaoxahexacosan-1-amine was obtained as yellow oil. LC/MS: mass calcd. for C24H₄₂N₂O₁: 534.27, found: 535.45 [M+H]⁺.

### Step 3: Synthesis of benzyl N-[26-(4-nitrophenoxy)-3,6,9,12,15,18,21,24-octaoxahexacosan-1-yl]carbamate

To a stirred solution of 26-(4-nitrophenoxy)-3,6,9,12,15,18,21,24-octaoxahexacosan-1-amine (920.00 mg, 1.72 mmol, 1.00 equiv) in DCM (6.00 mL) was added benzyl 2,5-dioxopyrrolidin-1-yl carbonate (514.66 mg, 2.07 mmol, 1.20 equiv) and DIEA (667.25 mg, 5.16 mmol, 3.00 equiv). The resulting mixture was stirred at room temperature for 1.0 h. The resulting mixture was concentrated under vacuum. The residue was purified by silica gel column chromatography (0-10% MeOH/DCM) to afford benzyl N-[26-(4-nitrophenoxy)-3,6,9,12,15,18,21,24-octaoxahexacosan-1-yl]carbamate (1.10 g, 95.58% yield) as yellow oil. LC/MS: mass calcd. For C32H₄₈N₂O₁₃: 668.31, found: 669.25 [M+H]⁺.

### Step 4: Synthesis of benzyl N-[26-(4-aminophenoxy)-3,6,9,12,15,18,21,24-octaoxahexacosan-1-yl]carbamate

To a stirred solution of benzyl N-[26-(4-nitrophenoxy)-3,6,9,12,15,18,21,24-octaoxahexacosan-1-yl]carbamate (1.10 g, 1.65 mmol, 1.00 equiv) in EtOH (6.00 mL) and H₂O(2.00 mL) was added Fe (1.38 g, 24.67 mmol, 15.00 equiv) and NH₄Cl (1.32 g, 24.68 mmol, 15.00 equiv). The resulting mixture was stirred at 70 degrees C. for 1.0 h. The resulting mixture was filtered, the filtrate was concentrated under reduced pressure. The residue was purified by silica gel column chromatography (0-10% MeOH/DCM) to afford benzyl N-[26-(4-aminophenoxy)-3,6,9,12,15,18,21,24-octaoxahexacosan-1-yl]carbamate (960.00 mg, 91.37% yield) as yellow oil. LC/MS: mass calcd. For C32H₅₀N₂O₁₁: 638.34, found: 639.35 [M+H]⁺.

### Step 5: Synthesis of benzyl N-[26-(4-{4-[2-(4-fluoro-2,6-dimethylphenoxy)-5-(2-hydroxypropan-2-yl)phenyl]-6-methyl-7-oxo-1H-pyrrolo[2,3-c]pyridine-2-amido}phenoxy)-3,6,9,12,15,18,21,24-octaoxahexacosan-1-yl]carbamate

The procedure was the same as methyl 4-(4-{4-[(tert-butoxycarbonyl)amino]-1-methylpyrrole-2-amido}-1-methylimidazole-2-amido)-1-methylpyrrole-2-carboxylate (Example 2 Step 3). 930.00 mg of benzyl N-[26-(4-aminophenoxy)-3,6,9,12,15,18,21,24-octaoxahexacosan-1-yl]carbamate was used, 1.50 g of benzyl N-[26-(4-{4-[2-(4-fluoro-2,6-dimethylphenoxy)-5-(2-hydroxypropan-2-yl)phenyl]-6-methyl-7-oxo-1H-pyrrolo[2,3-c]pyridine-2-amido}phenoxy)-3,6,9,12,15,18,21,24-octaoxahexacosan-1-yl]carbamate was obtained as yellow oil (94.93% yield). LC/MS: mass calcd. for C58H₇₃FN₄O₁₅: 1084.50, found: 1086.05 [M+H]⁺.

### Step 6: Synthesis of N-{4-[(26-amino-3,6,9,12,15,18,21,24-octaoxahexacosan-1-yl)oxy]phenyl}-4-[2-(4-fluoro-2,6-dimethylphenoxy)-5-(2-hydroxypropan-2-yl)phenyl]-6-methyl-7-oxo-1H-pyrrolo[2,3-c]pyridine-2-carboxamide

To a stirred solution of benzyl N-[26-(4-{4-[2-(4-fluoro-2,6-dimethylphenoxy)-5-(2-hydroxypropan-2-yl)phenyl]-6-methyl-7-oxo-1H-pyrrolo[2,3-c]pyridine-2-amido}phenoxy)-3,6,9,12,15,18,21,24-octaoxahexacosan-1-yl]carbamate (1.50 g, 1.38 mmol, 1.00 equiv) in DMF (8.00 mL) was added Pd/C (150.00 mg, 10% w/w). The resulting mixture was stirred at room temperature for 3.0 h under H₂ atmosphere. The resulting mixture was filtered and lyophilized directly to afford N-{4-[(26-amino-3,6,9,12,15,18,21,24-octaoxahexacosan-1-yl)oxy]phenyl}-4-[2-(4-fluoro-2,6-dimethylphenoxy)-5-(2-hydroxypropan-2-yl)phenyl]-6-methyl-7-oxo-1H-pyrrolo[2,3-c]pyridine-2-carboxamide (1.10 g, 83.68% yield) as yellow solid. LC/MS: mass calcd. For C₅₀H₆₇FN₄O₁₃: 950.46, found: 951.40 [M+H]⁺.

### Step 7: Synthesis of Compound 241

The procedure was the same as N-(5-{[2-({2-[(2-{[26-(4-{1-ethyl-4-[2-(4-fluoro-2,6-dimethylphenoxy)-5-(2-hydroxypropan-2-yl)phenyl]-6-methyl-7-oxopyrrolo[2,3-c]pyridine-2-amido}phenoxy)-3,6,9,12,15,18,21,24-octaoxahexacosan-1-yl]carbamoyl}ethyl)carbamoyl]-1-methylimidazol-4-yl}carbamoyl)ethyl]carbamoyl}-1-methylpyrrol-3-yl)-1-methyl-4-(3-{[1-methyl-4-(1-methylimidazole-2-amido)pyrrol-2-yl]formamido}propanamido)imidazole-2-carboxamide. 100.00 mg of 3-({1-methyl-4-[3-({4-[1-methyl-4-(3-{[1-methyl-4-(1-methylimidazole-2-amido)pyrrol-2-yl]formamido}propanamido)imidazole-2-amido]-1-[14-(2,5,8,11-tetraoxatridecan-13-yl)-2,5,8,11-tetraoxa-14-azanonadecan-19-yl]pyrrol-2-yl}formamido)propanamido]imidazol-2-yl}formamido)propanoic acid was used, 16.90 mg of desired product was obtained as white solid (9.43% yield). HRMS: mass calcd. for C₁₀₈H₁₅₃FN₂₀O₂₉: 2213.1096, found: 2214.1118 [M+H]⁺.

### Example 29. Synthesis of N-(5-{[2-({2-[(2-{[26-(4-{4-[2-(4-fluoro-2,6-dimethylphenoxy)-5-(2-hydroxypropan-2-yl)phenyl]-6-methyl-7-oxo-1H-pyrrolo[2,3-c]pyridine-2-amido}phenoxy)-3,6,9,12,15,18,21,24-octaoxahexacosan-1-yl]carbamoyl]ethyl)carbamoyl]-1-methylimidazol-4-yl}carbamoyl)ethyl]carbamoyl}-1-[26-(2,5,8,11,14,17,20,23-octaoxapentacosan-25-yl)-2,5,8,11,14,17,20,23-octaoxa-26-azahentriacontan-31-yl]pyrrol-3-yl)-1-methyl-4-(3-{[1-methyl-4-(1-methylimidazole-2-amido)pyrrol-2-yl]formamido]propanamido)imidazole-2-carboxamide (Compound 242)

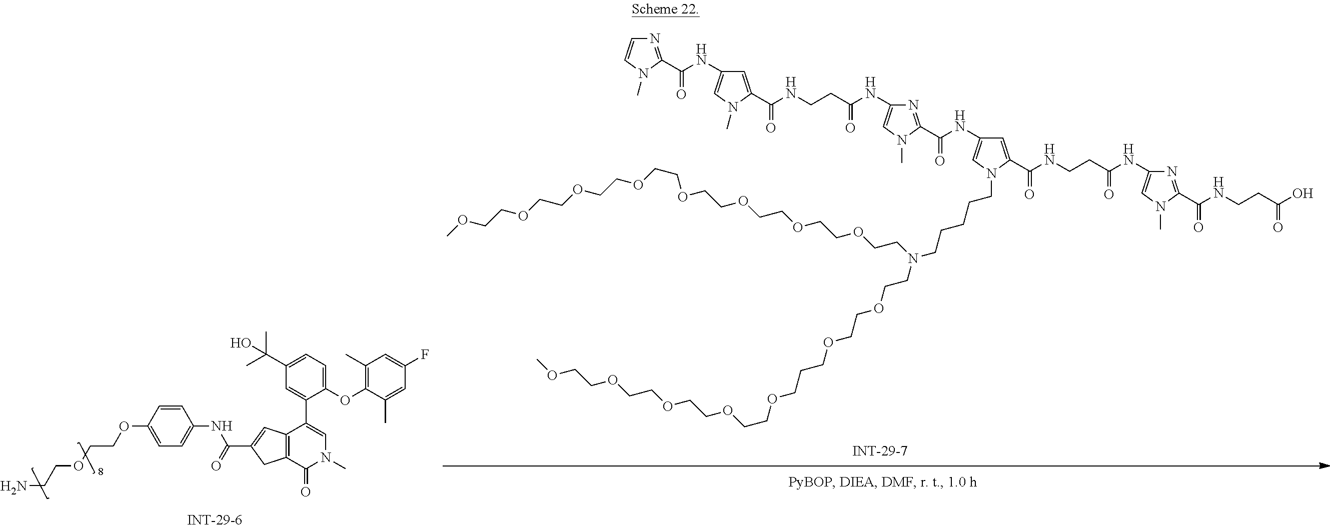

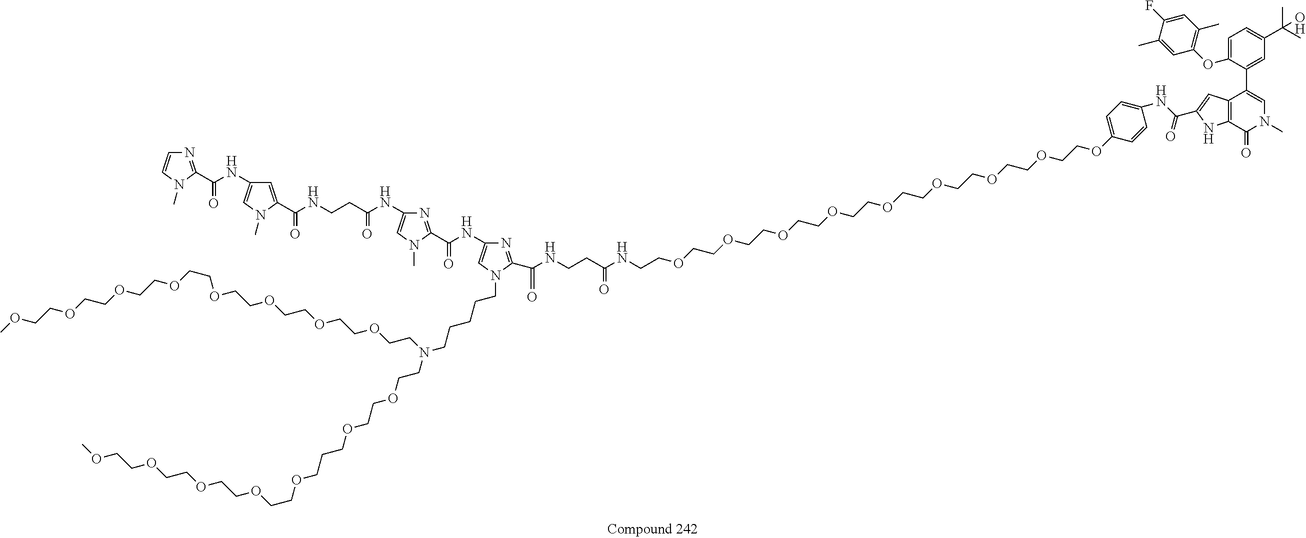 [SMILES: COCCOCCOCCOCCOCCCOCCOCCN(CCCCCn1cc(NC(=O)c2nc(NC(=O)CCNC(=O)c3cc(NC(=O)c4nccn4C)cn3C)cn2C)nc1C(=O)NCCC(=O)NCCOCCOCCOCCOCCOCCOCCOCCOCCOc1ccc(NC(=O)c2cc3c(-c4cc(C(C)(C)O)ccc4Oc4cc(C)c(F)cc4C)cn(C)c(=O)c3[nH]2)cc1)CCOCCOCCOCCOCCOCCOCCOCCOC]

The procedure was the same as N-(5-{[2-({2-[(2-{[26-(4-{1-ethyl-4-[2-(4-fluoro-2,6-dimethylphenoxy)-5-(2-hydroxypropan-2-yl)phenyl]-6-methyl-7-oxopyrrolo[2,3-c]pyridine-2-amido}phenoxy)-3,6,9,12,15,18,21,24-octaoxahexacosan-1-yl]carbamoyl}ethyl)carbamoyl]-1-methylimidazol-4-yl}carbamoyl)ethyl]carbamoyl}-1-methylpyrrol-3-yl)-1-methyl-4-(3-{[1-methyl-4-(1-methylimidazole-2-amido)pyrrol-2-yl]formamido}propanamido)imidazole-2-carboxamide (Example 20). 100.00 mg of N-{4-[(26-amino-3,6,9,12,15,18,21,24-octaoxahexacosan-1-yl)oxy]phenyl}-4-[2-(4-fluoro-2,6-dimethylphenoxy)-5-(2-hydroxypropan-2-yl)phenyl]-6-methyl-7-oxo-1H-pyrrolo[2,3-c]pyridine-2-carboxamide was used, 51.90 mg of desired product was obtained as white solid (17.84% yield). HRMS: mass calcd. for C₁₂₄H₁₈₅FN₂₀O₃₇: 2565.3194, found: 2566.3198 [M+H]⁺.

### Example 30. Synthesis of N-(1-{4-[bis(2,5,8,11-tetraoxatridecan-13-yl)carbamoyl]butyl]-5-{[2-({2-[(2-{[26-(4-{4-[2-(4-fluoro-2,6-dimethylphenoxy)-5-(2-hydroxypropan-2-yl)phenyl]-6-methyl-7-oxo-1H-pyrrolo[2,3-c]pyridine-2-amido}phenoxy)-3,6,9,12,15,18,21,24-octaoxahexacosan-1-yl]carbamoyl]ethyl)carbamoyl]-1-methylimidazol-4-yl]carbamoyl)ethyl]carbamoyl]pyrrol-3-yl)-1-methyl-4-(3-{[1-methyl-4-(1-methylimidazole-2-amido)pyrrol-2-yl]formamido]propanamido)imidazole-2-carboxamide (Compound 243) and N-(1-{4-[bis(2,5,8,11-tetraoxatridecan-13-yl) carbamoyl]butyl]-5-{[2-({2-[(2-{[26-(4-{4-[2-(4-fluoro-2,6-dimethylphenoxy)-5-(prop-1-en-2-yl)phenyl]-6-methyl-7-oxo-1H-Pyrrolo[2,3-c]pyridine-2-amido}phenoxy)-3,6,9,12,15,18,21,24-octaoxahexacosan-1-yl]carbamoyl]ethyl)carbamoyl]-1-methylimidazol-4-yl}carbamoyl)ethyl]carbamoyl]pyrrol-3-yl)-1-methyl-4-(3-{[1-methyl-4-(1-methylimidazole-2-amido)pyrrol-2-yl]formamido]propanamido)imidazole-2-carboxamide (Compound 249)

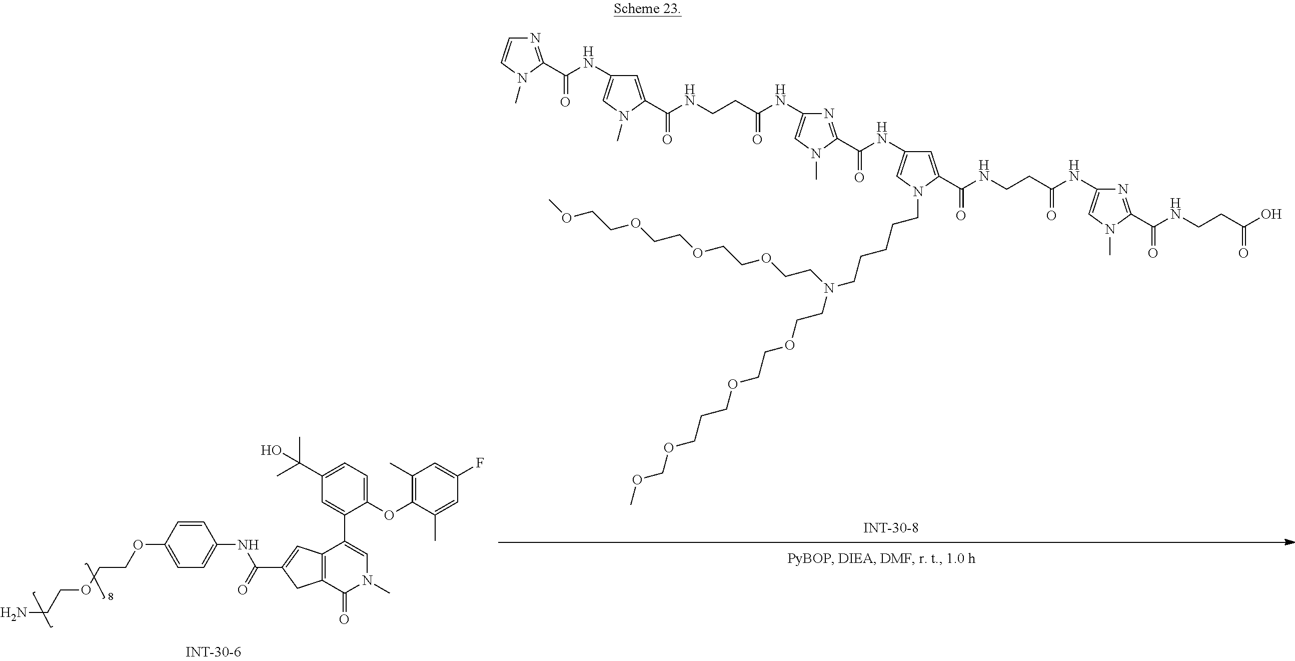

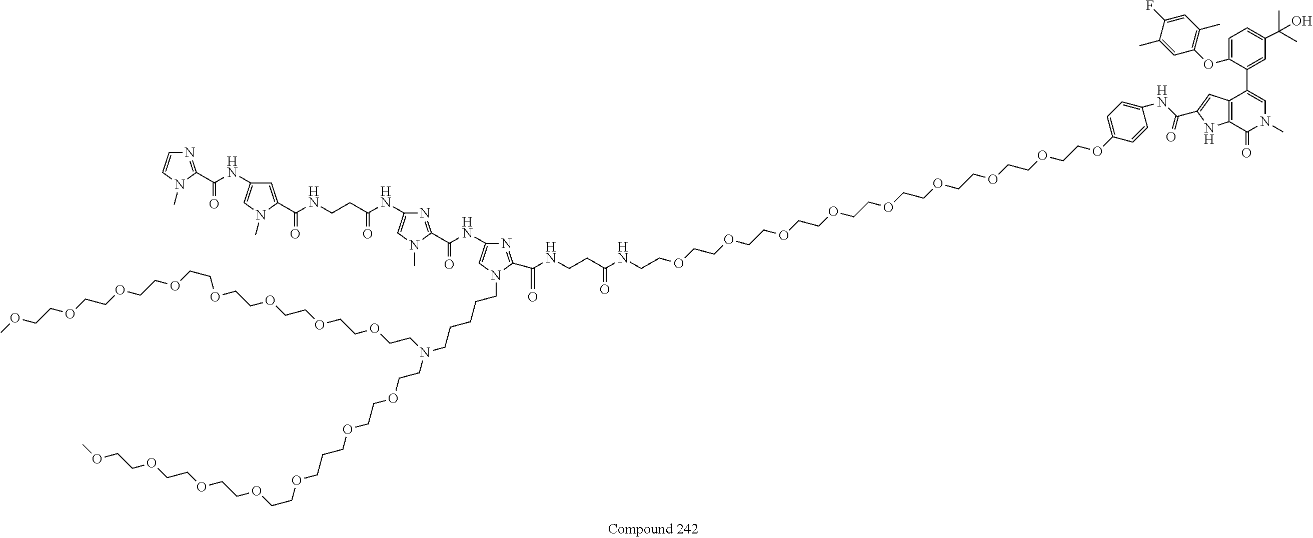 [SMILES: COCCOCCOCCOCCOCCCOCCOCCN(CCCCCn1cc(NC(=O)c2nc(NC(=O)CCNC(=O)c3cc(NC(=O)c4nccn4C)cn3C)cn2C)nc1C(=O)NCCC(=O)NCCOCCOCCOCCOCCOCCOCCOCCOCCOc1ccc(NC(=O)c2cc3c(-c4cc(C(C)(C)O)ccc4Oc4cc(C)c(F)cc4C)cn(C)c(=O)c3[nH]2)cc1)CCOCCOCCOCCOCCOCCOCCOCCOC]

To a stirred solution of 3-[(4-{3-[(1-{4-[bis(2,5,8,11-tetraoxatridecan-13-yl)carbamoyl]butyl}-4-[1-methyl-4-(3-{[1-methyl-4-(1-methylimidazole-2-amido)pyrrol-2-yl]formamido}propanamido)imidazole-2-amido]pyrrol-2-yl)formamido]propanamido}-1-methylimidazol-2-yl)formamido]propanoic acid (130.00 mg, 0.10 mmol, 1.00 equiv) in DMF (3.00 mL) was added PyBOP (78.34 mg, 0.15 mmol, 1.50 equiv), DIEA (38.91 mg, 0.30 mmol, 3.00 equiv) and N-{4-[(26-amino-3,6,9,12,15,18,21,24-octaoxahexacosan-1-yl)oxy]phenyl}-4-[2-(4-fluoro-2,6-dimethylphenoxy)-5-(2-hydroxypropan-2-yl)phenyl]-6-methyl-7-oxo-1H-pyrrolo[2,3-c]pyridine-2-carboxamide (104.99 mg, 0.11 mmol, 1.10 equiv) in portions at 0 degrees C. The resulting mixture was stirred for 1.0 h at room temperature. The resulting mixture was purified by reverse phase column directly with the following conditions: column, C18 silica gel; mobile phase, ACN in water (0.05% TFA), 5% to 93% gradient in 10 min; detector, UV 254 nm. The fractions were combined and concentrated. 150.00 mg of N-(1-{4-[bis(2,5,8,11-tetraoxatridecan-13-yl)carbamoyl]butyl}-5-{[2-({2-[(2-{[26-(4-{4-[2-(4-fluoro-2,6-dimethylphenoxy)-5-(2-hydroxypropan-2-yl)phenyl]-6-methyl-7-oxo-1H-pyrrolo[2,3-c]pyridine-2-amido}phenoxy)-3,6,9,12,15,18,21,24-octaoxahexacosan-1-yl]carbamoyl}ethyl)carbamoyl]-1-methylimidazol-4-yl}carbamoyl)ethyl]carbamoyl}pyrrol-3-yl)-1-methyl-4-(3-{[1-methyl-4-(1-methylimidazole-2-amido)pyrrol-2-yl]formamido}propanamido)imidazole-2-carboxamide and N-(1-{4-[bis(2,5,8,11-tetraoxatridecan-13-yl)carbamoyl]butyl}-5-{[2-({2-[(2-{[26-(4-{4-[2-(4-fluoro-2,6-dimethylphenoxy)-5-(prop-1-en-2-yl)phenyl]-6-methyl-7-oxo-1H-pyrrolo[2,3-c]pyridine-2-amido}phenoxy)-3,6,9,12,15,18,21,24-octaoxahexacosan-1-yl]carbamoyl}ethyl)carbamoyl]-1-methylimidazol-4-yl}carbamoyl)ethyl]carbamoyl}pyrrol-3-yl)-1-methyl-4-(3-{[1-methyl-4-(1-methylimidazole-2-amido)pyrrol-2-yl]formamido}propanamido)imidazole-2-carboxamide was obtained. Both of them were dissolved in DMF (2.0 mL), filtered and the filtrates in DMF were purified by Prep-HPLC with the following conditions: Column: XBridge Prep Phenyl OBD Column, 19*250 mm, 5 μm; Mobile Phase A: Water (10 mmol/L NH₄HCO₃+0.1% NH₃·H₂O), Mobile Phase B: ACN; Flow rate: 25 mL/min; Gradient: 32% B to 57% B in 18 min, 57% B; Wave Length: 254 n; RT1(min): 13.3, 16.97(min). The fractions were combined and lyophilized directly. N-(1-{4-[bis(2,5,8,11-tetraoxatridecan-13-yl)carbamoyl]butyl}-5-{[2-({2-[(2-{[26-(4-{4-[2-(4-fluoro-2,6-dimethylphenoxy)-5-(2-hydroxypropan-2-yl)phenyl]-6-methyl-7-oxo-1H-pyrrolo[2,3-c]pyridine-2-amido}phenoxy)-3,6,9,12,15,18,21,24-octaoxahexacosan-1-yl]carbamoyl}ethyl)carbamoyl]-1-methylimidazol-4-yl}carbamoyl)ethyl]carbamoyl}pyrrol-3-yl)-1-methyl-4-(3-{[1-methyl-4-(1-methylimidazole-2-amido)pyrrol-2-yl]formamido}propanamido)imidazole-2-carboxamide (13.70 mg, 5.96%) was obtained as white solid. HRMS: mass calcd. for C₁₀₈H₁₅₁FN₂₀O₃₀: 2227.0889, found: 2228.0912 [M+H]⁺. N-(1-{4-[bis(2,5,8,11-tetraoxatridecan-13-yl)carbamoyl]butyl}-5-{[2-({2-[(2-{[26-(4-{4-[2-(4-fluoro-2,6-dimethylphenoxy)-5-(prop-1-en-2-yl)phenyl]-6-methyl-7-oxo-1H-pyrrolo[2,3-c]pyridine-2-amido}phenoxy)-3,6,9,12,15,18,21,24-octaoxahexacosan-1-yl]carbamoyl}ethyl)carbamoyl]-1-methylimidazol-4-yl}carbamoyl)ethyl]carbamoyl}pyrrol-3-yl)-1-methyl-4-(3-{[1-methyl-4-(1-methylimidazole-2-amido)pyrrol-2-yl]formamido}propanamido)imidazole-2-carboxamide (26.40 mg, 32.97% yield) was obtained as white solid. HRMS: mass calcd. for C₁₀₈H₁₄₉FN₂₀O₂₉: 2209.0783, found: 2210.0817 [M+H]⁺.

Examples 31-33 were synthesized according to the procedure of Example 30.

### Example 31. Synthesis of N-(5-{[2-({2-[(2-{[26-(4-{4-[2-(4-fluoro-2,6-dimethylphenoxy)-5-(2-hydroxypropan-2-yl)phenyl]-6-methyl-7-oxo-1H-pyrrolo[2,3-c]pyridine-2-amido}phenoxy)-3,6,9,12,15,18,21,24-octaoxahexacosan-1-yl]carbamoyl]ethyl)carbamoyl]-1-methylimidazol-4-yl]carbamoyl)ethyl]carbamoyl]-1-methylpyrrol-3-yl)-1-methyl-4-(3-{[4-(1-methylimidazole-2-amido)-1-[14-(2,5,8,11-tetraoxatridecan-13-yl)-2,5,8,11-tetraoxa-14-azanonadecan-19-yl]pyrrol-2-yl]formamido]propanamido)imidazole-2-carboxamide (Compound 245)

100.00 mg of N-{4-[(26-amino-3,6,9,12,15,18,21,24-octaoxahexacosan-1-yl)oxy]phenyl}-4-[2-(4-fluoro-2,6-dimethylphenoxy)-5-(2-hydroxypropan-2-yl)phenyl]-6-methyl-7-oxo-1H-pyrrolo[2,3-c]pyridine-2-carboxamide was used, 17.30 mg of desired product was obtained as white solid (6.88% yield). HRMS: mass calcd. for C₁₀₈H₁₅₃FN₂₀O₂₉: 2213.1096, found: 2214.1126 [M+H]⁺.

### Example 32. Synthesis of N-(5-{[2-({2-[(2-{[26-(4-{4-[2-(4-fluoro-2,6-dimethylphenoxy)-5-(2-hydroxypropan-2-yl])phenyl]-6-methyl-7-oxo-1H-pyrrolo[2,3-c]pyridine-2-amido}phenoxy)-3,6,9,12,15,18,21,24-octaoxahexacosan-1-yl]carbamoyl]ethyl)carbamoyl]-1-methylimidazol-4-yl}carbamoyl)ethyl]carbamoyl]-1-methylpyrrol-3-yl)-1-methyl-4-(3-{[4-(1-methylimidazole-2-amido)-1-[26-(2,5,8,11,14,17,20,23-octaoxapentacosan-25-yl)-2,5,8,11,14,17,20,23-octaoxa-26-azahentriacontan-31-yl]pyrrol-2-yl]formamido}propanamido)imidazole-2-carboxamide (Compound 246)

60.00 mg of 3-({1-methyl-4-[3-({1-methyl-4-[1-methyl-4-(3-{[4-(1-methylimidazole-2-amido)-1-[26-(2,5,8,11,14,17,20,23-octaoxapentacosan-25-yl)-2,5,8,11,14,17,20,23-octaoxa-26-azahentriacontan-31-yl]pyrrol-2-yl]formamido}propanamido)imidazole-2-amido]pyrrol-2-yl}formamido)propanamido]imidazol-2-yl}formamido)propanoic acid was used, 2.80 mg of desired product was obtained as yellow solid (2.73% yield). HRMS: mass calcd. for C₁₂₄H₁₈₅FN₂₀O₃₇: 2565.3194, found: 2566.3151 [M+H]⁺.

### Example 33. Synthesis of N-[1-(6-azidohexyl)-5-{[2-({2-[(2-{[26-(4-{4-[2-(4-fluoro-2,6-dimethylphenoxy)-5-(2-hydroxypropan-2-yl)phenyl]-6-methyl-7-oxo-1H-pyrrolo[2,3-c]pyridine-2-amido}phenoxy)-3,6,9,12,15,18,21,24-octaoxahexacosan-1-yl]carbamoyl]ethyl)carbamoyl]-1-methylimidazol-4-yl}carbamoyl)ethyl]carbamoyl}pyrrol-3-yl]-1-methyl-4-(3-{[1-methyl-4-(1-methylimidazole-2-amido)pyrrol-2-yl]formamido}propanamido)imidazole-2-carboxamide (Compound 263)

230.00 mg of 3-{[4-(3-{[1-(6-azidohexyl)-4-[1-methyl-4-(3-{[1-methyl-4-(1-methylimidazole-2-amido)pyrrol-2-yl]formamido}propanamido)imidazole-2-amido]pyrrol-2-yl]formamido}propanamido)-1-methylimidazol-2-yl]formamido}propanoic acid was used, 150.00 mg of desired product was obtained as yellow oil. 50.00 mg of it was purified by Prep-HPLC, 11.30 mg of desired product was obtained as white solid. HRMS: mass calcd. For C₉₁H₁₁₇FN₂₂O₂₁: 1872.8748, found: 1873.8771 [M+H]⁺.

### Example 34. Synthesis of N-[1-(6-aminohexyl)-5-{[2-({2-[(2-{[26-(4-{4-[2-(4-fluoro-2,6-dimethylphenoxy)-5-(2-hydroxypropan-2-yl)phenyl]-6-methyl-7-oxo-1H-pyrrolo[2,3-c]pyridine-2-amido}phenoxy)-3,6,9,12,15,18,21,24-octaoxahexacosan-1-yl]carbamoyl]ethyl)carbamoyl]-1-methylimidazol-4-yl]carbamoyl)ethyl]carbamoyl]}pyrrol-3-yl]-1-methyl-4-(3-{[1-methyl-4-(1-methylimidazole-2-amido)pyrrol-2-yl]formamido}propanamido)imidazole-2-carboxamide (Compound 264)

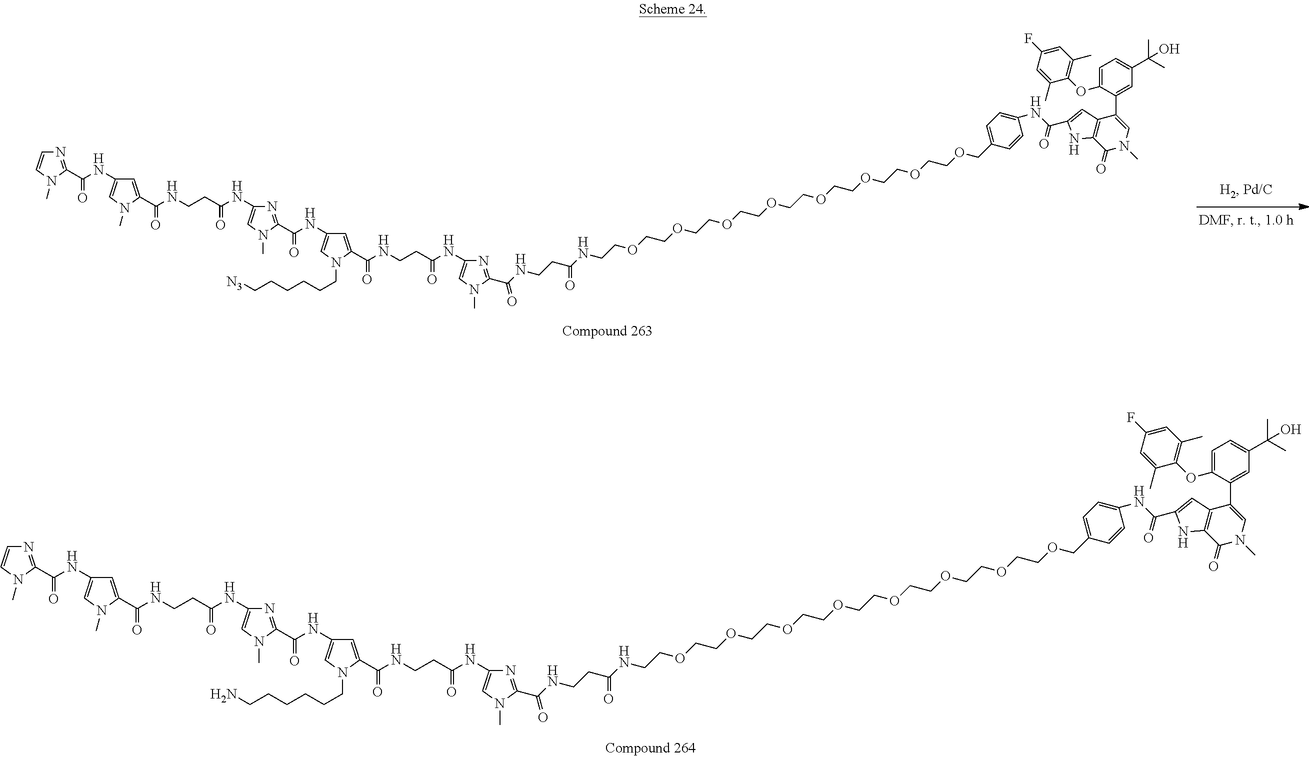 [SMILES: Cc1cc(F)cc(C)c1Oc1ccc(C(C)(C)O)cc1-c1cn(C)c(=O)c2[nH]c(C(=O)Nc3ccc(COCCOCCOCCOCCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5CCCCCCN=[N+]=[N-])cn4C)cc3)cc12 ; Cc1cc(F)cc(C)c1Oc1ccc(C(C)(C)O)cc1-c1cn(C)c(=O)c2[nH]c(C(=O)Nc3ccc(COCCOCCOCCOCCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5CCCCCCN)cn4C)cc3)cc12]

To a solution of N-[1-(6-azidohexyl)-5-{[2-({2-[(2-{[26-(4-{4-[2-(4-fluoro-2,6-dimethylphenoxy)-5-(2-hydroxypropan-2-yl)phenyl]-6-methyl-7-oxo-1H-pyrrolo[2,3-c]pyridine-2-amido}phenoxy)-3,6,9,12,15,18,21,24-octaoxahexacosan-1-yl]carbamoyl}ethyl)carbamoyl]-1-methylimidazol-4-yl}carbamoyl)ethyl]carbamoyl}pyrrol-3-yl]-1-methyl-4-(3-{[1-methyl-4-(1-methylimidazole-2-amido)pyrrol-2-yl]formamido}propanamido)imidazole-2-carboxamide (90.00 mg, 0.05 mmol, 1.00 equiv) in DMF (4.00 mL) was added Pd/C (30.00 mg, 33.3% w/w). Then the reaction was stirred for 1.0 h at room temperature under H₂ atmosphere. The mixture was filtrated and the filtrate was concentrated under vacuum. The residue was dissolved in DMF (3.00 mL) and filtered and the filtrate in DMF was purified by Prep-HPLC with the following conditions: Column: XBridge Prep Phenyl OBD Column, 19*250 mm, 5 μm; Mobile Phase A: Water(10 mmol/L NH₄HCO₃+0.1% NH₃·H₂O), Mobile Phase B: ACN; Flow rate: 25 mL/min; Gradient: 35% B to 60% B in 15 min, 60% B; Wave Length: 254 n; RT1(min): 13. The fractions were combined and lyophilized directly. N-[1-(6-aminohexyl)-5-{[2-({2-[(2-{[26-(4-{4-[2-(4-fluoro-2,6-dimethylphenoxy)-5-(2-hydroxypropan-2-yl)phenyl]-6-methyl-7-oxo-1H-pyrrolo[2,3-c]pyridine-2-amido}phenoxy)-3,6,9,12,15,18,21,24-octaoxahexacosan-1-yl]carbamoyl}ethyl)carbamoyl]-1-methylimidazol-4-yl}carbamoyl)ethyl]carbamoyl}pyrrol-3-yl]-1-methyl-4-(3-{[1-methyl-4-(1-methylimidazole-2-amido)pyrrol-2-yl]formamido}propanamido)imidazole-2-carboxamide (10.00 mg, 10.87% yield) was obtained as white solid. HRMS: mass calcd. For C₉₁H₁₁₉FN₂₀O₂₁: 1846.8843, found: 1847.8887 [M+H]⁺.

### Example 35. Synthesis of 4-(3-aminopropanamido)-N-(5-{[2-({2-[(2-[26-(4-{4-[2-(4-fluoro-2,6-dimethylphenoxy)-5-(2-hydroxypropan-2-yl)phenyl]-6-methyl-7-oxo-1H-pyrrolo[2,3-c]pyridine-2-amido}phenoxy)-3,6,9,12,15,18,21,24-octaoxahexacosan-1-yl]carbamoyl]ethyl)carbamoyl]-1-methylimidazol-4-yl]carbamoyl)ethyl]carbamoyl]-1-methylpyrrol-3-yl)-1-methylimidazole-2-carboxamide (Compound 199)

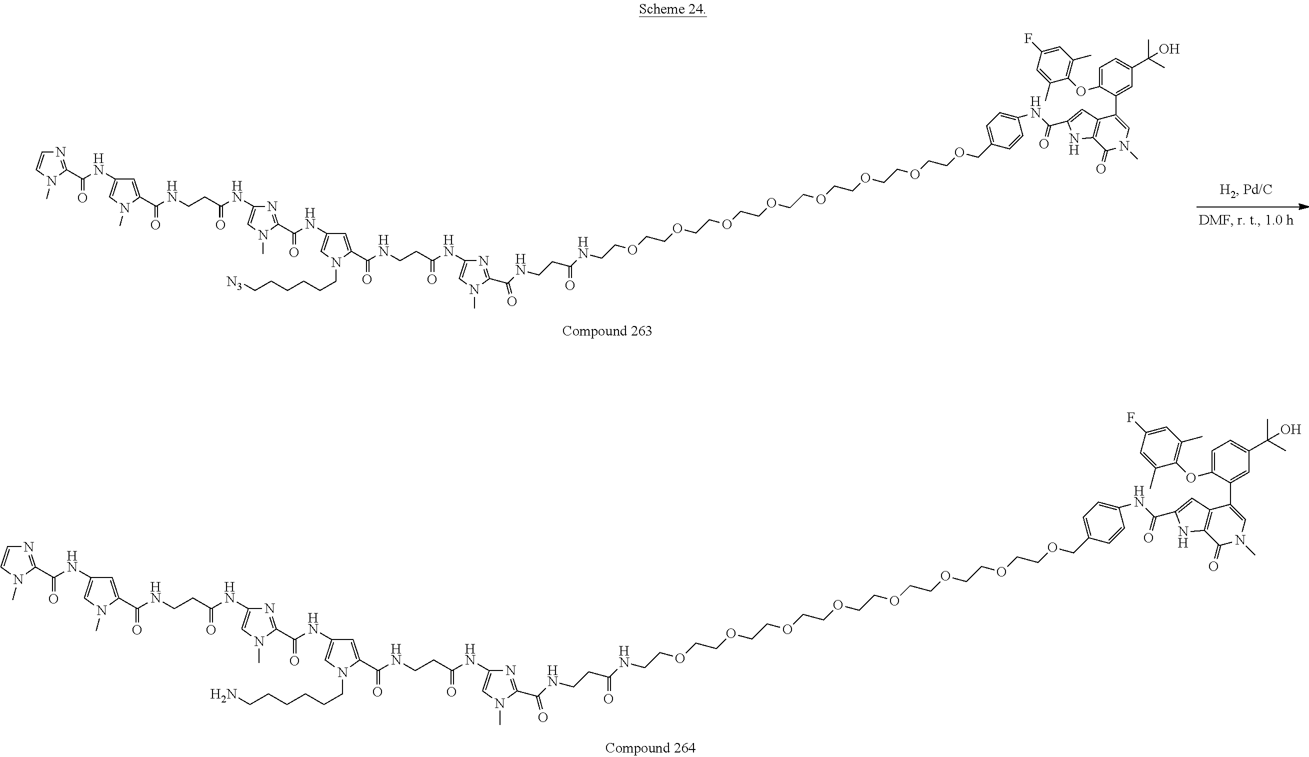 [SMILES: Cc1cc(F)cc(C)c1Oc1ccc(C(C)(C)O)cc1-c1cn(C)c(=O)c2[nH]c(C(=O)Nc3ccc(COCCOCCOCCOCCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5CCCCCCN=[N+]=[N-])cn4C)cc3)cc12 ; Cc1cc(F)cc(C)c1Oc1ccc(C(C)(C)O)cc1-c1cn(C)c(=O)c2[nH]c(C(=O)Nc3ccc(COCCOCCOCCOCCOCCOCCOCCOCCNC(=O)CCNC(=O)c4nc(NC(=O)CCNC(=O)c5cc(NC(=O)c6nc(NC(=O)CCNC(=O)c7cc(NC(=O)c8nccn8C)cn7C)cn6C)cn5CCCCCCN)cn4C)cc3)cc12]

### Step 1: Synthesis of methyl 3-{[4-(3-{[4-(4-{3-[(tert-butoxycarbonyl)amino] propanamido}-1-methylimidazole-2-amido)-1-methylpyrrol-2-yl]formamido}propanamido)-1-methylimidazol-2-yl]formamido}propanoate

The procedure was the same as ethyl 4-(1-methylimidazole-2-amido)-1H-pyrrole-2-carboxylate (INT-459-2). 380.00 mg of methyl 3-{[4-(3-aminopropanamido)-1-methylimidazol-2-yl]formamido}propanoate was used, 790.00 mg of methyl 3-{[4-(3-{[4-(4-{3-[(tert-butoxycarbonyl)amino]propanamido}-1-methylimidazole-2-amido)-1-methylpyrrol-2-yl]formamido}propanamido)-1-methylimidazol-2-yl]formamido}propanoate was obtained as light yellow solid (82.27% yield). LC/MS: mass calcd. for C₃₁H₄₃N₁₁O₉: 713.32, found: 714.55 [M+H]⁺.

### Step 2: Synthesis of 3-{[4-(3-{[4-(4-{3-[(tert-butoxycarbonyl)amino] propanamido}-1-methylimidazole-2-amido)-1-methylpyrrol-2-yl]formamido}propanamido)-1-methylimidazol-2-yl]formamido}propanoic Acid

The procedure was the same as 4-[3-[(tert-butoxycarbonyl)amino] propanamido]-1-methylimidazole-2-carboxylic acid (Example 1 Step 3), but the reaction temperature was room temperature. 790.00 mg of methyl 3-{[4-(3-{[4-(4-{3-[(tert-butoxycarbonyl)amino]propanamido}-1-methylimidazole-2-amido)-1-methylpyrrol-2-yl]formamido}propanamido)-1-methylimidazol-2-yl]formamido}propanoate was used, 700.00 mg of 3-{[4-(3-{[4-(4-{3-[(tert-butoxycarbonyl)amino]propanamido}-1-methylimidazole-2-amido)-1-methylpyrrol-2-yl]formamido}propanamido)-1-methylimidazol-2-yl]formamido}propanoic acid was obtained as light yellow solid (86.77% yield). LC/MS: mass calcd. for C₃₀H₄₁N₁₁O₉: 699.31, found: 701.50 [M+H]⁺.

### Step 3: Synthesis of 3-({4-[3-({4-[4-(3-aminopropanamido)-1-methylimidazole-2-amido)-1-methylpyrrol-2-yl}formamido)propanamido]-1-methylimidazol-2-yl}formamido)propanoic Acid

The procedure was the same as methyl 4-[4-(3-aminopropanamido)-1-methylimidazole-2-amido]-1-methylpyrrole-2-carboxylate hydrochloride (Example 4 Step 1), but the reaction solvent was 4M HCl in dioxane/DCM (1:1) and the reaction time was 1.0 h. 650.00 mg of 3-{[4-(3-{[4-(4-{3-[(tert-butoxycarbonyl)amino]propanamido}-1-methylimidazole-2-amido)-1-methylpyrrol-2-yl]formamido}propanamido)-1-methylimidazol-2-yl]formamido}propanoic acid was used, 650.00 mg crude of desired product was obtained as yellow oil. LC/MS: mass calcd. for C₂₅H₃₃N₁₁O₇: 599.26, found: 600.25 [M+H]⁺.

### Step 4: Synthesis of 3-({4-[3-({4-[4-(3-{[(9H-fluoren-9-ylmethoxy) carbonyl]amino}propanamido)-1-methylimidazole-2-amido)-1-methylpyrrol-2-yl}formamido)propanamido]-1-methylimidazol-2-yl}formamido)propanoic Acid

To a stirred solution of 3-({4-[3-({4-[4-(3-aminopropanamido)-1-methylimidazole-2-amido]-1-methylpyrrol-2-yl}formamido)propanamido]-1-methylimidazol-2-yl}formamido)propanoic acid (600.00 mg, 1.00 mmol, 1.00 equiv) in THF (10.00 mL) and H₂O (2.00 mL) was added 2,5-dioxopyrrolidin-1-yl 9H-fluoren-9-ylmethyl carbonate (337.55 mg, 1.00 mmol, 1.00 equiv) and NaHCO₃ (420.30 mg, 5.00 mmol, 5.00 equiv) at room temperature. The resulting mixture was stirred for 2.0 h at room temperature. After reaction, the resulting mixture was concentrated under reduced pressure. The pH was adjusted to 3˜4, then extracted by EA (3×20 mL), the organic phases were combined and concentrated. This resulted in 3-({4-[3-({4-[4-(3-{[(9H-fluoren-9-ylmethoxy)carbonyl]amino}propanamido)-1-methylimidazole-2-amido]-1-methylpyrrol-2-yl}formamido)propanamido]-1-methylimidazol-2-yl}formamido)propanoic acid (800.00 mg, crude) as light yellow solid. The crude product was used in the next step directly without further purification. LC/MS: mass calcd. For C₄₀H₄₃N₁₁O₉: 821.32, found: 822.55 [M+H]⁺.

### Step 5: Synthesis of 9H-fluoren-9-ylmethyl N-[2-({2-[(5-{[2-({2-[(2-{[26-(4-{[(benzyloxy)carbonyl]amino}phenoxy)-3,6,9,12,15,18,21,24-octaoxahexacosan-1-yl]carbamoyl}ethyl)carbamoyl]-1-methylimidazol-4-yl}carbamoyl)ethyl]carbamoyl}-1-methylpyrrol-3-yl)carbamoyl]-1-methylimidazol-4-yl}carbamoyl)ethyl]carbamate

The procedure was the same as ethyl 1-methyl-4-[3-methyl-3-({1-methyl-4-[1-methyl-4-(3-{[1-methyl-4-(1-methylimidazole-2-amido)pyrrol-2-yl] formamido}propanamido)imidazole-2-amido]pyrrol-2-yl}formamido)butanamido]imidazole-2-carboxylate, but the reaction time was 1.0 h. 650.00 mg of 3-({4-[3-({4-[4-(3-{[(9H-fluoren-9-ylmethoxy)carbonyl]amino}propanamido)-1-methylimidazole-2-amido]-1-methylpyrrol-2-yl}formamido)propanamido]-1-methylimidazol-2-yl}formamido)propanoic acid was used, 740.00 mg of desired product was obtained as light yellow oil (57.72% yield). LC/MS: mass calcd. for C₇₂H₉₁N₁₃O₁₉: 1441.66, found: 722.40 [M/2+H]⁺.

### Step 6: Synthesis of 9H-fluoren-9-ylmethyl N-[2-({2-[(5-{[2-({2-[(2-{[26-(4-aminophenoxy)-3,6,9,12,15,18,21,24-octaoxahexacosan-1-yl]carbamoyl}ethyl)carbamoyl]-1-methylimidazol-4-yl}carbamoyl)ethyl]carbamoyl}-1-methylpyrrol-3-yl)carbamoyl]-1-methylimidazol-4-yl}carbamoyl)ethyl]carbamate

To a solution of 9H-fluoren-9-ylmethyl N-[2-({2-[(5-{[2-({2-[(2-{[26-(4-{[(benzyloxy)carbonyl]amino}phenoxy)-3,6,9,12,15,18,21,24-octaoxahexacosan-1-yl]carbamoyl}ethyl) carbamoyl]-1-methylimidazol-4-yl}carbamoyl)ethyl]carbamoyl}-1-methylpyrrol-3-yl)carbamoyl]-1-methylimidazol-4-yl}carbamoyl)ethyl]carbamate (730.00 mg, 0.51 mmol, 1.00 equiv) in DMF (8.00 mL) was added Pd/C (146.00 mg, 20% w/w) in a 50 mL round-bottom flask. The mixture was hydrogenated at room temperature for 2.0 h under H₂ atmosphere using a hydrogen balloon. The resulting mixture was filtered, the filter cake was washed with MeOH (3×5 mL). The filtrate was concentrated under reduced pressure and lyophilized to afford 9H-fluoren-9-ylmethyl N-[2-({2-[(5-{[2-({2-[(2-{[26-(4-aminophenoxy)-3,6,9,12,15,18,21,24-octaoxahexacosan-1-yl]carbamoyl}ethyl)carbamoyl]-1-methylimidazol-4-yl}carbamoyl)ethyl]carbamoyl}-1-methylpyrrol-3-yl)carbamoyl]-1-methylimidazol-4-yl}carbamoyl)ethyl]carbamate (600.00 mg, crude) as dark yellow oil. The crude product was used in the next step directly without further purification. LC/MS: mass calcd. For C₆₄H₈₅N₁₃O₁₇: 1307.62, found: 655.40 [M/2+H]⁺.

### Step 7: Synthesis of 9H-fluoren-9-ylmethyl N-[2-({2-[(5-{[2-({2-[(2-{[26-(4-{4-[2-(4-fluoro-2,6-dimethylphenoxy)-5-(2-hydroxypropan-2-yl)phenyl]-6-methyl-7-oxo-1H-pyrrolo[2,3-c]pyridine-2-amido}phenoxy)-3,6,9,12,15,18,21,24-octaoxahexacosan-1-yl]carbamoyl}ethyl)carbamoyl]-1-methylimidazol-4-yl}carbamoyl)ethyl]carbamoyl}-1-methylpyrrol-3-yl)carbamoyl]-1-methylimidazol-4-yl}carbamoyl)ethyl]carbamate

The procedure was the same as ethyl 4-(1-methylimidazole-2-amido)-1H-pyrrole-2-carboxylate (INT-459-2). 500.00 mg of 9H-fluoren-9-ylmethyl N-[2-({2-[(5-{[2-({2-[(2-{[26-(4-aminophenoxy)-3,6,9,12,15,18,21,24-octaoxahexacosan-1-yl]carbamoyl}ethyl)carbamoyl]-1-methylimidazol-4-yl}carbamoyl)ethyl]carbamoyl}-1-methylpyrrol-3-yl)carbamoyl]-1-methylimidazol-4-yl}carbamoyl)ethyl]carbamate was used, 420.00 mg of 9H-fluoren-9-ylmethyl N-[2-({2-[(5-{[2-({2-[(2-{[26-(4-{4-[2-(4-fluoro-2,6-dimethylphenoxy)-5-(2-hydroxypropan-2-yl)phenyl]-6-methyl-7-oxo-1H-pyrrolo[2,3-c]pyridine-2-amido}phenoxy)-3,6,9,12,15,18,21,24-octaoxahexacosan-1-yl]carbamoyl}ethyl)carbamoyl]-1-methylimidazol-4-yl}carbamoyl)ethyl] carbamoyl}-1-methylpyrrol-3-yl)carbamoyl]-1-methylimidazol-4-yl}carbamoyl)ethyl]carbamate was obtained as light yellow solid (50.10% yield). LC/MS: mass calcd. for C₉₀H₁₀₈FN₁₅O₂₁: 1753.78, found: 869.65 [(M-OH)/2+H]⁺.

### Step 8: Synthesis of Compound 199

To a stirred solution of 9H-fluoren-9-ylmethyl N-[2-({2-[(5-{[2-({2-[(2-{[26-(4-{4-[2-(4-fluoro-2,6-dimethylphenoxy)-5-(2-hydroxypropan-2-yl)phenyl]-6-methyl-7-oxo-1H-pyrrolo[2,3-c]pyridine-2-amido}phenoxy)-3,6,9,12,15,18,21,24-octaoxahexacosan-1-yl]carbamoyl}ethyl)carbamoyl]-1-methylimidazol-4-yl}carbamoyl)ethyl]carbamoyl}-1-methylpyrrol-3-yl)carbamoyl]-1-methylimidazol-4-yl}carbamoyl)ethyl]carbamate (400.00 mg, 0.23 mmol, 1.00 equiv) in DMF (3.00 mL) was added piperidine (0.30 mL) dropwise at room temperature. The resulting mixture was stirred for 1.0 h at room temperature. After reaction, the reaction mixture was filtered and the filtration in DMF (3.00 mL) was purified by Perp-HPLC: Column: XBridge Shield RP18 OBD Column, 19*250 mm, 10 μm; Mobile Phase A: Water (0.05% TFA), Mobile Phase B: ACN; Flow rate: 25 mL/min; Gradient: 40% B to 50% B in 10 min, 50% B; Wave Length: 254 nm; RT1(min): 9.15; Number Of Runs: 0. The fractions were combined and lyophilized directly. This resulted in 4-(3-aminopropanamido)-N-(5-{[2-({2-[(2-{[26-(4-{4-[2-(4-fluoro-2,6-dimethylphenoxy)-5-(2-hydroxypropan-2-yl)phenyl]-6-methyl-7-oxo-1H-pyrrolo[2,3-c]pyridine-2-amido}phenoxy)-3,6,9,12,15,18,21,24-octaoxahexacosan-1-yl]carbamoyl}ethyl)carbamoyl]-1-methylimidazol-4-yl}carbamoyl)ethyl]carbamoyl}-1-methylpyrrol-3-yl)-1-methylimidazole-2-carboxamide (121.60 mg, 33.67% yield) as white solid. HRMS: mass calcd. For C₇₅H₉₈FN₁₅O₁₉: 1531.7147, found: 1532.7252 [M+H]⁺.

### Example 36. Synthesis of N-(5-{[26-(4-{4-[2-(4-fluoro-2,6-dimethylphenoxy)-5-(2-hydroxypropan-2-yl)phenyl]-6-methyl-7-oxo-1H-pyrrolo[2,3-c]pyridine-2-amido}phenoxy)-3,6,9,12,15,18,21,24-octaoxahexacosan-1-yl]carbamoyl]-1-methylpyrrol-3-yl)-1-methyl-4-{1-methyl-4-[1-methyl-4-(1-methylimidazole-2-amido)pyrrole-2-amido]pyrrole-2-amido}imidazole-2-carboxamide (Compound 214)

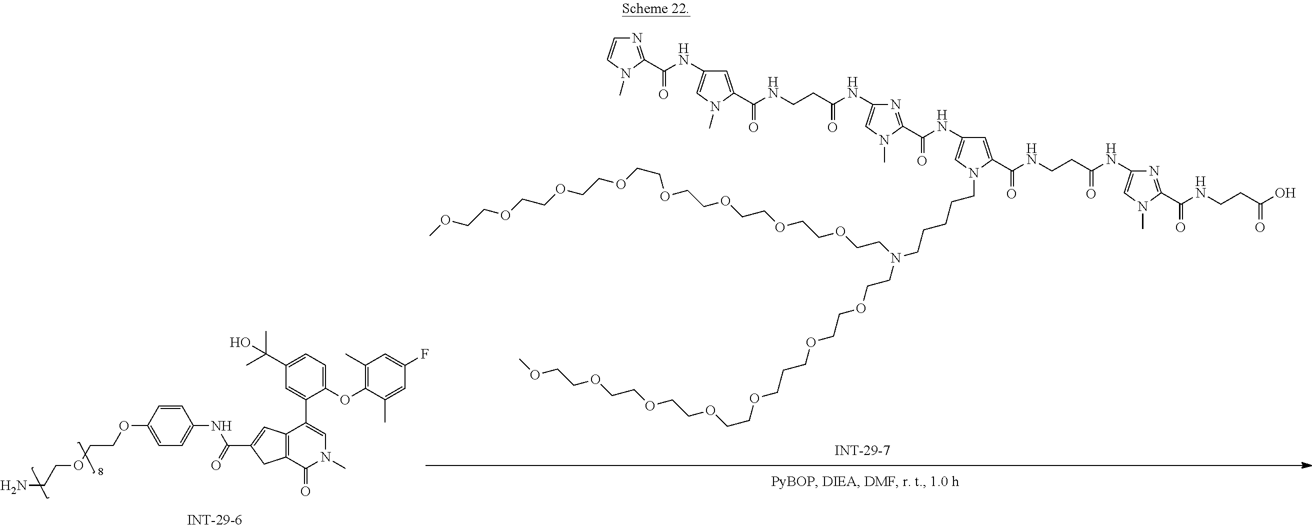

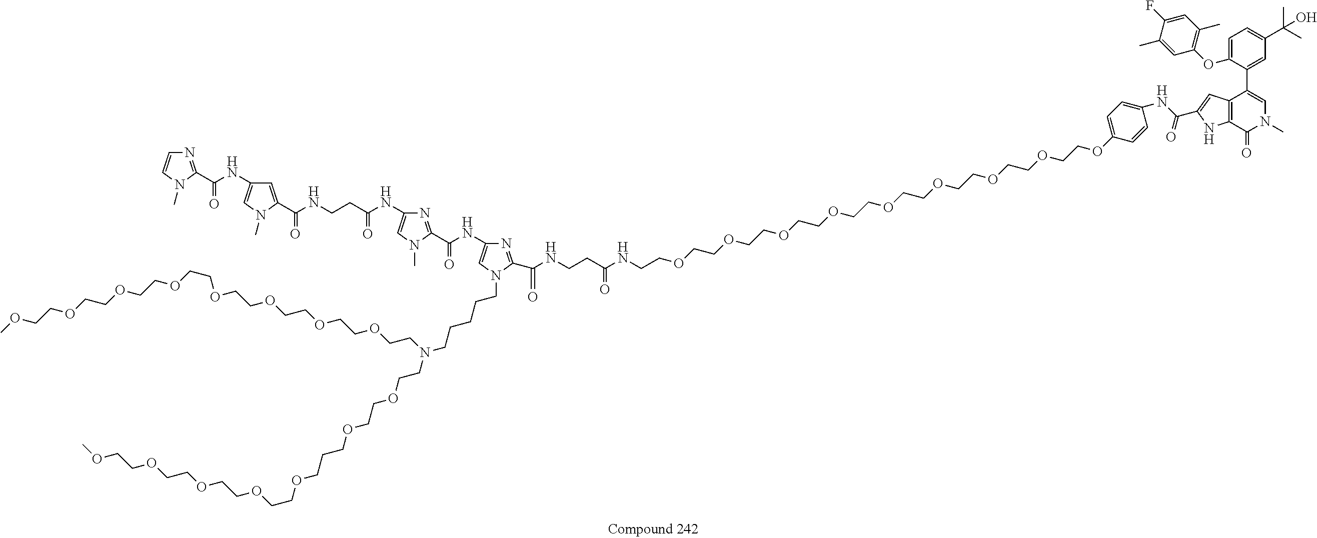 [SMILES: COCCOCCOCCOCCOCCCOCCOCCN(CCCCCn1cc(NC(=O)c2nc(NC(=O)CCNC(=O)c3cc(NC(=O)c4nccn4C)cn3C)cn2C)nc1C(=O)NCCC(=O)NCCOCCOCCOCCOCCOCCOCCOCCOCCOc1ccc(NC(=O)c2cc3c(-c4cc(C(C)(C)O)ccc4Oc4cc(C)c(F)cc4C)cn(C)c(=O)c3[nH]2)cc1)CCOCCOCCOCCOCCOCCOCCOCCOC]

### Step 1: Synthesis of benzyl N-{4-[(26-{[1-methyl-4-(1-methyl-4-{1-methyl-4-[1-methyl-4-(1-methylimidazole-2-amido)pyrrole-2-amido)pyrrole-2-amido}imidazole-2-amido)pyrrol-2-yl]formamido}-3,6,9,12,15,18,21,24-octaoxahexacosan-1-yl)oxy]phenyl}carbamate

The procedure was the same as methyl 1-methyl-4-(1-methyl-4-{1-methyl-4-[1-methyl-4-(1-methylimidazole-2-amido)pyrrole-2-amido]pyrrole-2-amido}imidazole-2-amido)pyrrole-2-carboxylate (INT-450-11). 170.00 mg of benzyl N-{4-[(26-amino-3,6,9,12,15,18,21,24-octaoxahexacosan-1-yl)oxy]phenyl}carbamate was used, 150.00 mg of benzyl N-{4-[(26-{[1-methyl-4-(1-methyl-4-{1-methyl-4-[1-methyl-4-(1-methylimidazole-2-amido)pyrrole-2-amido]pyrrole-2-amido}imidazole-2-amido)pyrrol-2-yl]formamido}-3,6,9,12,15,18,21,24-octaoxahexacosan-1-yl)oxy]phenyl}carbamate was obtained as yellow solid (43.31% yield). LC/MS: mass calcd. for C₆₀H₇₇N₁₃O₁₆: 1235.56, found: 619.25 [M/2+H]⁺.

### Step 2: Synthesis of N-(5-{[26-(4-aminophenoxy)-3,6,9,12,15,18,21,24-octaoxahexacosan-1-yl]carbamoyl}-1-methylpyrrol-3-yl)-1-methyl-4-[1-methyl-4-[1-methyl-4-(1-methylimidazole-2-amido)pyrrole-2-amido)pyrrole-2-amido}imidazole-2-carboxamide

The procedure was the same as 9H-fluoren-9-ylmethyl N-[2-({2-[(5-{[2-({2-[(2-{[26-(4-aminophenoxy)-3,6,9,12,15,18,21,24-octaoxahexacosan-1-yl]carbamoyl}ethyl)carbamoyl]-1-methylimidazol-4-yl}carbamoyl)ethyl]carbamoyl}-1-methylpyrrol-3-yl)carbamoyl]-1-methylimidazol-4-yl}carbamoyl)ethyl]carbamate (Example 35 Step 6). 150.00 mg of benzyl N-{4-[(26-{[1-methyl-4-(1-methyl-4-{1-methyl-4-[1-methyl-4-(1-methylimidazole-2-amido)pyrrole-2-amido]pyrrole-2-amido}imidazole-2-amido)pyrrol-2-yl]formamido}-3,6,9,12,15,18,21,24-octaoxahexacosan-1-yl)oxy]phenyl}carbamate was used, 110.00 mg crude of N-(5-{[26-(4-aminophenoxy)-3,6,9,12,15,18,21,24-octaoxahexacosan-1-yl]carbamoyl}-1-methylpyrrol-3-yl)-1-methyl-4-{1-methyl-4-[1-methyl-4-(1-methylimidazole-2-amido)pyrrole-2-amido]pyrrole-2-amido}imidazole-2-carboxamide was obtained as yellow oil. LC/MS: mass calcd. for C₅₂H₇₁N₁₃O₁₄: 1101.52, found: 552.25 [M/2+H]⁺.

### Step 3: Synthesis of Compound 214

The procedure was the same as N-(5-{[2-({2-[(2-{[26-(4-{1-ethyl-4-[2-(4-fluoro-2,6-dimethylphenoxy)-5-(2-hydroxypropan-2-yl)phenyl]-6-methyl-7-oxopyrrolo[2,3-c]pyridine-2-amido}phenoxy)-3,6,9,12,15,18,21,24-octaoxahexacosan-1-yl]carbamoyl}ethyl)carbamoyl]-1-methylimidazol-4-yl}carbamoyl)ethyl]carbamoyl}-1-methylpyrrol-3-yl)-1-methyl-4-(3-{[1-methyl-4-(1-methylimidazole-2-amido)pyrrol-2-yl]formamido}propanamido)imidazole-2-carboxamide (Example 20). 100.00 mg of N-(5-{[26-(4-aminophenoxy)-3,6,9,12,15,18,21,24-octaoxahexacosan-1-yl]carbamoyl}-1-methylpyrrol-3-yl)-1-methyl-4-{1-methyl-4-[1-methyl-4-(1-methylimidazole-2-amido)pyrrole-2-amido]pyrrole-2-amido}imidazole-2-carboxamide was used, 41.20 mg of desired product was obtained as light yellow solid (29.33% yield). HRMS: mass calcd. for C₇₈H₉₄FN₁₅O₁₈: 1547.6885, found: 1548.7098 [M+H]⁺.

### Example 37. Synthesis of N-[5-({2-[(5-{[26-(4-{4-[2-(4-fluoro-2,6-dimethylphenoxy)-5-(2-hydroxypropan-2-yl)phenyl]-6-methyl-7-oxo-1H-pyrrolo[2,3-c]pyridine-2-amido}phenoxy)-3,6,9,12,15,18,21,24-octaoxahexacosan-1-yl]carbamoyl}-1-methylpyrrol-3-yl)carbamoyl]-1-methylimidazol-4-yl]carbamoyl)-1-methylpyrrol-3-yl]-2-(1-methylimidazol-2-yl)-3H-1,3-benzodiazole-5-carboxamide (Compound 215)

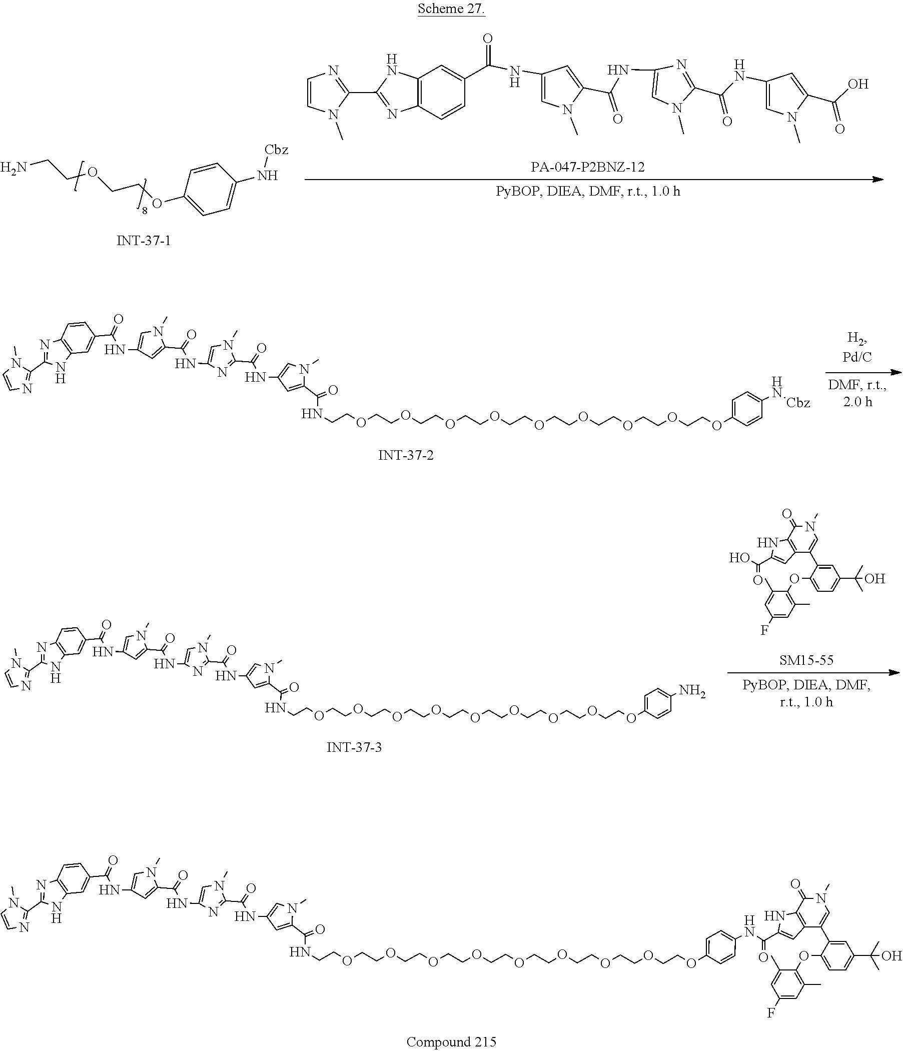

### Step 1: Synthesis of benzyl N-{4-[(26-{[1-methyl-4-(1-methyl-4-{1-methyl-4-[2-(1-methylimidazol-2-yl)-3H-1,3-benzodiazole-5-amido]pyrrole-2-amido}imidazole-2-amido)pyrrol-2-yl]formamido}-3,6,9,12,15,18,21,24-octaoxahexacosan-1-yl)oxy]phenyl}carbamate

The procedure was the same as methyl 4-(4-{4-[(tert-butoxycarbonyl)amino]-1-methylpyrrole-2-amido}-1-methylimidazole-2-amido)-1-methylpyrrole-2-carboxylate (Example 2 Step 3). 210.00 mg of benzyl N-{4-[(26-amino-3,6,9,12,15,18,21,24-octaoxahexacosan-1-yl)oxy]phenyl}carbamate was used, 320.00 mg of desired product was obtained as yellow solid (79.11% yield). LC/MS: mass calcd. for C₆₁H₇₅N₁₃O₁₅: 1229.55, found: 1231.10 [M+H]⁺.

### Step 2: Synthesis of N-[5-({2-[(5-{[26-(4-aminophenoxy)-3,6,9,12,15,18,21,24-octaoxahexacosan-1-yl]carbamoyl}-1-methylpyrrol-3-yl)carbamoyl]-1-methylimidazol-4-yl}carbamoyl)-1-methylpyrrol-3-yl]-2-(1-methylimidazol-2-yl)-3H-1,3-benzodiazole-5-carboxamide

The procedure was the same as 9H-fluoren-9-ylmethyl N-[2-({2-[(5-{[2-({2-[(2-{[26-(4-aminophenoxy)-3,6,9,12,15,18,21,24-octaoxahexacosan-1-yl]carbamoyl}ethyl)carbamoyl]-1-methylimidazol-4-yl}carbamoyl)ethyl]carbamoyl}-1-methylpyrrol-3-yl)carbamoyl]-1-methylimidazol-4-yl}carbamoyl)ethyl]carbamate (Example 35 Step 6). 150.00 mg of benzyl N-{4-[(26-{[1-methyl-4-(1-methyl-4-{1-methyl-4-[2-(1-methylimidazol-2-yl)-3H-1,3-benzodiazole-5-amido]pyrrole-2-amido}imidazole-2-amido)pyrrol-2-yl]formamido}-3,6,9,12,15,18,21,24-octaoxahexacosan-1-yl)oxy]phenyl}carbamate was used, 120.00 mg crude of desired product was obtained as yellow oil. LC/MS: mass calcd. for C₅₃H₆₉N₁₃O₁₃: 1095.51, found: 1097.00 [M+H]⁺.

### Step 3: Synthesis of Compound 215

The procedure was the same as N-(5-{[2-({2-[(2-{[26-(4-{1-ethyl-4-[2-(4-fluoro-2,6-dimethylphenoxy)-5-(2-hydroxypropan-2-yl)phenyl]-6-methyl-7-oxopyrrolo[2,3-c]pyridine-2-amido}phenoxy)-3,6,9,12,15,18,21,24-octaoxahexacosan-1-yl]carbamoyl}ethyl)carbamoyl]-1-methylimidazol-4-yl}carbamoyl)ethyl]carbamoyl}-1-methylpyrrol-3-yl)-1-methyl-4-(3-{[1-methyl-4-(1-methylimidazole-2-amido)pyrrol-2-yl]formamido}propanamido)imidazole-2-carboxamide (Example 20). 120.00 mg of N-[5-({2-[(5-{[26-(4-aminophenoxy)-3,6,9,12,15,18,21,24-octaoxahexacosan-1-yl]carbamoyl}-1-methylpyrrol-3-yl)carbamoyl]-1-methylimidazol-4-yl}carbamoyl)-1-methylpyrrol-3-yl]-2-(1-methylimidazol-2-yl)-3H-1,3-benzodiazole-5-carboxamide was used, 7.10 mg of desired product was obtained as white solid (4.09% yield). HRMS: mass calcd. For C₇₉H₉₂FN₁₅O₁₇: 1541.6779, found: 1542.6900 [M+H]⁺.

### Example 38. Synthesis of N-[26-(4-{4-[2-(4-fluoro-2,6-dimethylphenoxy)-5-(2-hydroxypropan-2-yl)phenyl]-6-methyl-7-oxo-1H-pyrrolo[2,3-c]pyridine-2-amido}phenoxy)-3,6,9,12,15,18,21,24-octaoxahexacosan-1-yl]-1-methyl-4-[2-(1-{1-methyl-4-[1-methyl-4-(3-{[1-methyl-4-(1-methylimidazole-2-amido)pyrrol-2-yl]formamido}propanamido)imidazole-2-amido]pyrrole-2-amido}cyclopropyl)acetamido]imidazole-2-carboxamide (Compound 219)

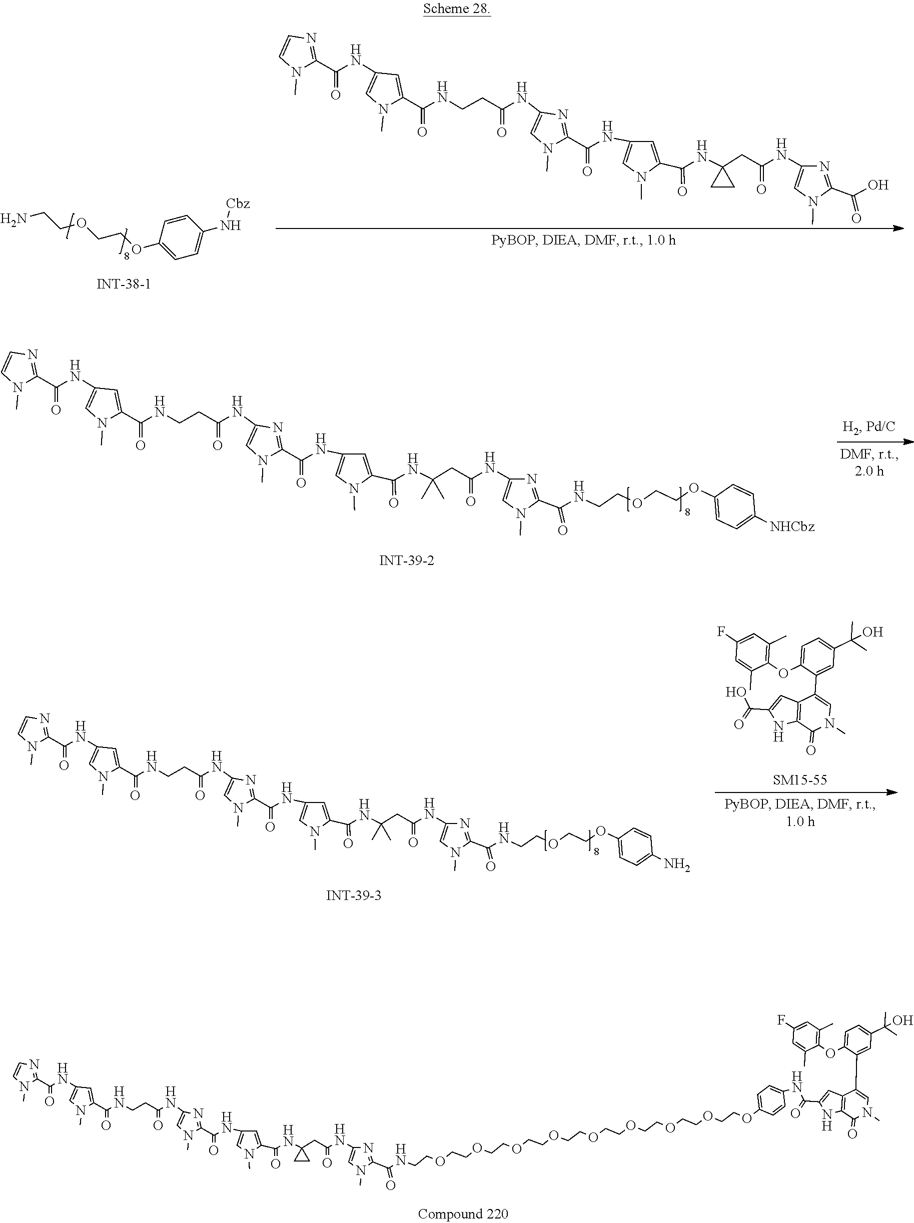

### Step 1: Synthesis of benzyl N-(4-{[26-({1-methyl-4-[2-(1-{1-methyl-4-[1-methyl-4-(3-{[1-methyl-4-(1-methylimidazole-2-amido)pyrrol-2-yl]formamido}propanamido)imidazole-2-amido)pyrrole-2-amido}cyclopropyl)acetamido]imidazol-2-yl}formamido)-3,6,9,12,15,18,21,24-octaoxahexacosan-1-yl]oxy}phenyl)carbamate

The procedure was the same as methyl 1-methyl-4-(1-methyl-4-{1-methyl-4-[1-methyl-4-(1-methylimidazole-2-amido)pyrrole-2-amido]pyrrole-2-amido}imidazole-2-amido)pyrrole-2-carboxylate. 155.00 mg of 1-methyl-4-[2-(1-{1-methyl-4-[1-methyl-4-(3-{[1-methyl-4-(1-methylimidazole-2-amido)pyrrol-2-yl]formamido}propanamido)imidazole-2-amido]pyrrole-2-amido}cyclopropyl)acetamido]imidazole-2-carboxylic acid was used, 172.00 mg of desired product was obtained as white solid (58.86% yield). LC/MS: mass calcd. for C₆₇H₈₈N₁₆O₁₈: 1404.65, found: 703.95 [M/2+H]⁺.

### Step 2: Synthesis of N-[26-(4-aminophenoxy)-3,6,9,12,15,18,21,24-octaoxahexacosan-1-yl]-1-methyl-4-[2-(1-[1-methyl-4-[1-methyl-4-(3-{[1-methyl-4-(1-methylimidazole-2-amido)pyrrol-2-yl]formamido}propanamido)imidazole-2-amido]pyrrole-2-amido}cyclopropyl)acetamido]imidazole-2-carboxamide

The procedure was the same as 9H-fluoren-9-ylmethyl N-[2-({2-[(5-{[2-({2-[(2-{[26-(4-aminophenoxy)-3,6,9,12,15,18,21,24-octaoxahexacosan-1-yl]carbamoyl}ethyl)carbamoyl]-1-methylimidazol-4-yl}carbamoyl)ethyl]carbamoyl}-1-methylpyrrol-3-yl)carbamoyl]-1-methylimidazol-4-yl}carbamoyl)ethyl]carbamate. 172.00 mg of benzyl N-(4-{[26-({1-methyl-4-[2-(1-{1-methyl-4-[1-methyl-4-(3-{[1-methyl-4-(1-methylimidazole-2-amido)pyrrol-2-yl]formamido}propanamido)imidazole-2-amido]pyrrole-2-amido}cyclopropyl)acetamido]imidazol-2-yl}formamido)-3,6,9,12,15,18,21,24-octaoxahexacosan-1-yl]oxy}phenyl)carbamate was used, 140.00 mg crude of desired product was obtained as yellow oil. LC/MS: mass calcd. for C₅₉H₈₂N₁₆O₁₆: 1270.61, found: 636.90 [M/2+H]⁺.

### Step 3: Synthesis of Compound 219

The procedure was the same as N-(5-{[2-({2-[(2-{[26-(4-{1-ethyl-4-[2-(4-fluoro-2,6-dimethylphenoxy)-5-(2-hydroxypropan-2-yl)phenyl]-6-methyl-7-oxopyrrolo[2,3-c]pyridine-2-amido}phenoxy)-3,6,9,12,15,18,21,24-octaoxahexacosan-1-yl]carbamoyl}ethyl)carbamoyl]-1-methylimidazol-4-yl}carbamoyl)ethyl]carbamoyl}-1-methylpyrrol-3-yl)-1-methyl-4-(3-{[1-methyl-4-(1-methylimidazole-2-amido)pyrrol-2-yl]formamido}propanamido)imidazole-2-carboxamide (Example 20). 140.00 mg of N-[26-(4-aminophenoxy)-3,6,9,12,15,18,21,24-octaoxahexacosan-1-yl]-1-methyl-4-[2-(1-{1-methyl-4-[1-methyl-4-(3-{[1-methyl-4-(1-methylimidazole-2-amido)pyrrol-2-yl]formamido}propanamido)imidazole-2-amido]pyrrole-2-amido}cyclopropyl)acetamido]imidazole-2-carboxamide was used, 36.40 mg of desired product was obtained as white solid (17.64% yield). HRMS: mass calcd. For C₈₅H₁₀₅FN₁₈O₂₀: 1716.7737, found: 1717.7741 [M+H]⁺.

### Example 39. Synthesis of N-[26-(4-{4-[2-(4-fluoro-2,6-dimethylphenoxy)-5-(2-hydroxypropan-2-yl)phenyl]-6-methyl-7-oxo-1H-Pyrrolo[2,3-c]pyridine-2-amido}phenoxy)-3,6,9,12,15,18,21,24-octaoxahexacosan-1-yl]-1-methyl-4-[3-methyl-3-({1-methyl-4-[1-methyl-4-(3-{[1-methyl-4-(1-methylimidazole-2-amido)pyrrol-2-yl]formamido}propanamido)imidazole-2-amido]pyrrol-2-yl]formamido)butanamido]imidazole-2-carboxamide (Compound 220)

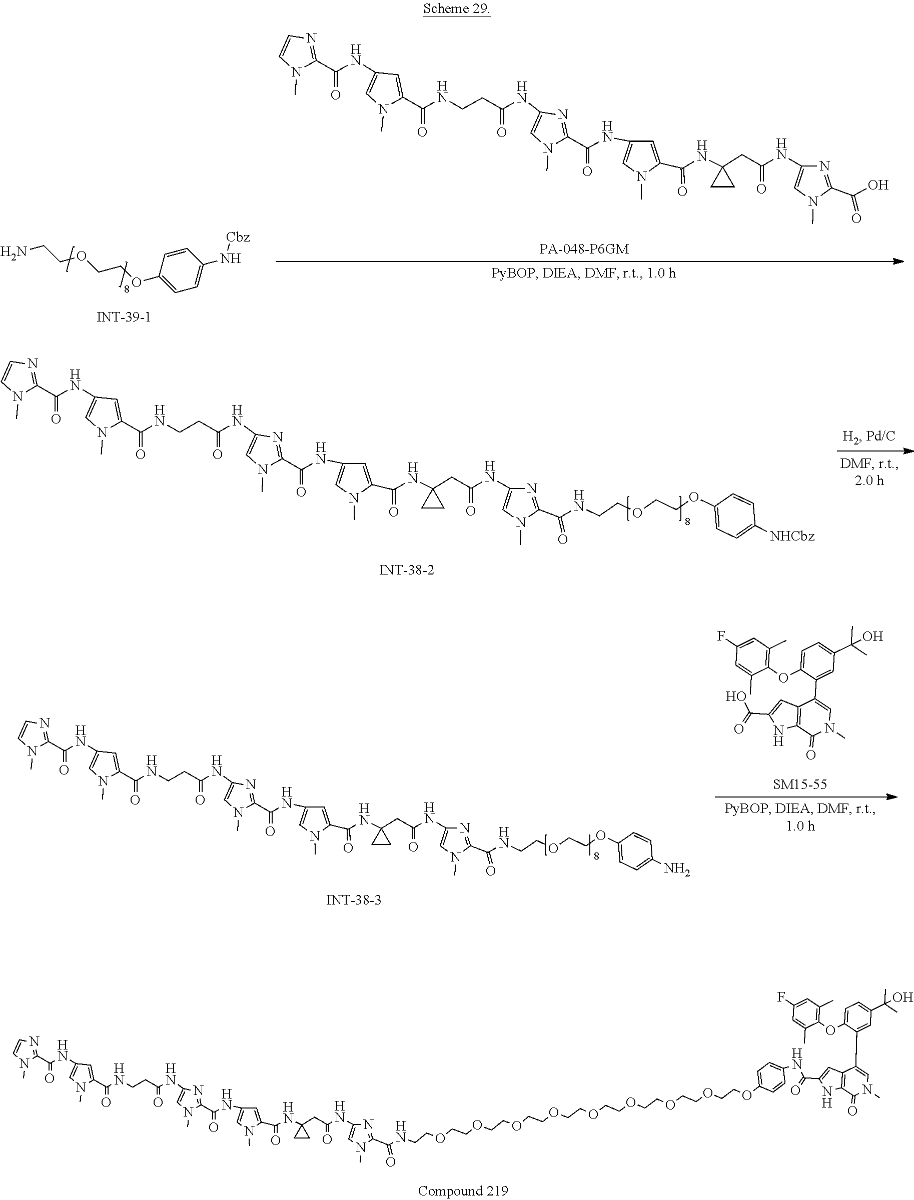

### Step 1: Synthesis of benzyl N-(4-{[26-([1-methyl-4-[3-methyl-3-([1-methyl-4-[1-methyl-4-(3-{[1-methyl-4-(1-methylimidazole-2-amido)pyrrol-2-yl]formamido}propanamido)imidazole-2-amido)pyrrol-2-yl}formamido)butanamido)imidazol-2-yl}formamido)-3,6,9,12,15,18,21,24-octaoxahexacosan-1-yl]oxy}phenyl)carbamate

The procedure was the same as methyl 1-methyl-4-(1-methyl-4-{1-methyl-4-[1-methyl-4-(1-methylimidazole-2-amido)pyrrole-2-amido]pyrrole-2-amido}imidazole-2-amido)pyrrole-2-carboxylate. 190.00 mg of 1-methyl-4-[3-methyl-3-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido) butanamido]imidazole-2-carboxylic acid was used, 270.00 mg of desired product was obtained as yellow oil (76.12% yield). LC/MS: mass calcd. for C₆₇H₉₀N₁₆O₁₈: 1406.66, found: 704.80 [M/2+H]⁺.

### Step 2: Synthesis of N-[26-(4-aminophenoxy)-3,6,9,12,15,18,21,24-octaoxahexacosan-1-yl]-1-methyl-4-[3-methyl-3-([1-methyl-4-[1-methyl-4-(3-{[1-methyl-4-(1-methylimidazole-2-amido)pyrrol-2-yl]formamido}propanamido)imidazole-2-amido)pyrrol-2-yl}formamido) butanamido]imidazole-2-carboxamide

The procedure was the same as 9H-fluoren-9-ylmethyl N-[2-({2-[(5-{[2-({2-[(2-{[26-(4-aminophenoxy)-3,6,9,12,15,18,21,24-octaoxahexacosan-1-yl]carbamoyl}ethyl)carbamoyl]-1-methylimidazol-4-yl}carbamoyl)ethyl]carbamoyl}-1-methylpyrrol-3-yl)carbamoyl]-1-methylimidazol-4-yl}carbamoyl)ethyl]carbamate (Example 35 Step 6). 250.00 mg of benzyl N-(4-{[26-({1-methyl-4-[3-methyl-3-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)butanamido]imidazol-2-yl}formamido)-3,6,9,12,15,18,21,24-octaoxahexacosan-1-yl]oxy}phenyl)carbamate was used, 250.00 mg crude of desired product was obtained as yellow oil. LC/MS: mass calcd. for C₅₉H₈₄N₁₆O₁₆: 1272.63, found: 637.55 [M/2+H]⁺.

### Step 3: Synthesis of Compound 220

The procedure was the same as N-(5-{[2-({2-[(2-{[26-(4-{1-ethyl-4-[2-(4-fluoro-2,6-dimethylphenoxy)-5-(2-hydroxypropan-2-yl)phenyl]-6-methyl-7-oxopyrrolo[2,3-c]pyridine-2-amido}phenoxy)-3,6,9,12,15,18,21,24-octaoxahexacosan-1-yl]carbamoyl}ethyl)carbamoyl]-1-methylimidazol-4-yl}carbamoyl)ethyl]carbamoyl}-1-methylpyrrol-3-yl)-1-methyl-4-(3-{[1-methyl-4-(1-methylimidazole-2-amido)pyrrol-2-yl]formamido}propanamido)imidazole-2-carboxamide (Example 20). 196.00 mg of N-[26-(4-aminophenoxy)-3,6,9,12,15,18,21,24-octaoxahexacosan-1-yl]-1-methyl-4-[3-methyl-3-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido) butanamido]imidazole-2-carboxamide was used, 69.00 mg of desired product was obtained as white solid (24.88% yield). HRMS: mass calcd. For C₈₅H₁₀₇FN₁₈O₂₀: 1718.7893, found: 1719.7988 [M+H]⁺

### Example 40. Synthesis of N-[26-(4-{4-[2-(4-fluoro-2,6-dimethylphenoxy)-5-(2-hydroxypropan-2-yl)penyl]-6-methyl-7-oxo-1H-pyrrolo[2,3-c]pyridine-2-amido}phenoxy)-3,6,9,12,15,18,21,24-octaoxahexacosan-1-yl]-1-methyl-4-(1-{1-methyl-4-[1-methyl-4-(3-{[1-methyl-4-(1-methylimidazole-2-amido)pyrrol-2-yl]formamido]propanamido)imidazole-2-amido]pyrrole-2-carbonyl}azetidine-3-amido)imidazole-2-carboxamide (Compound 222)

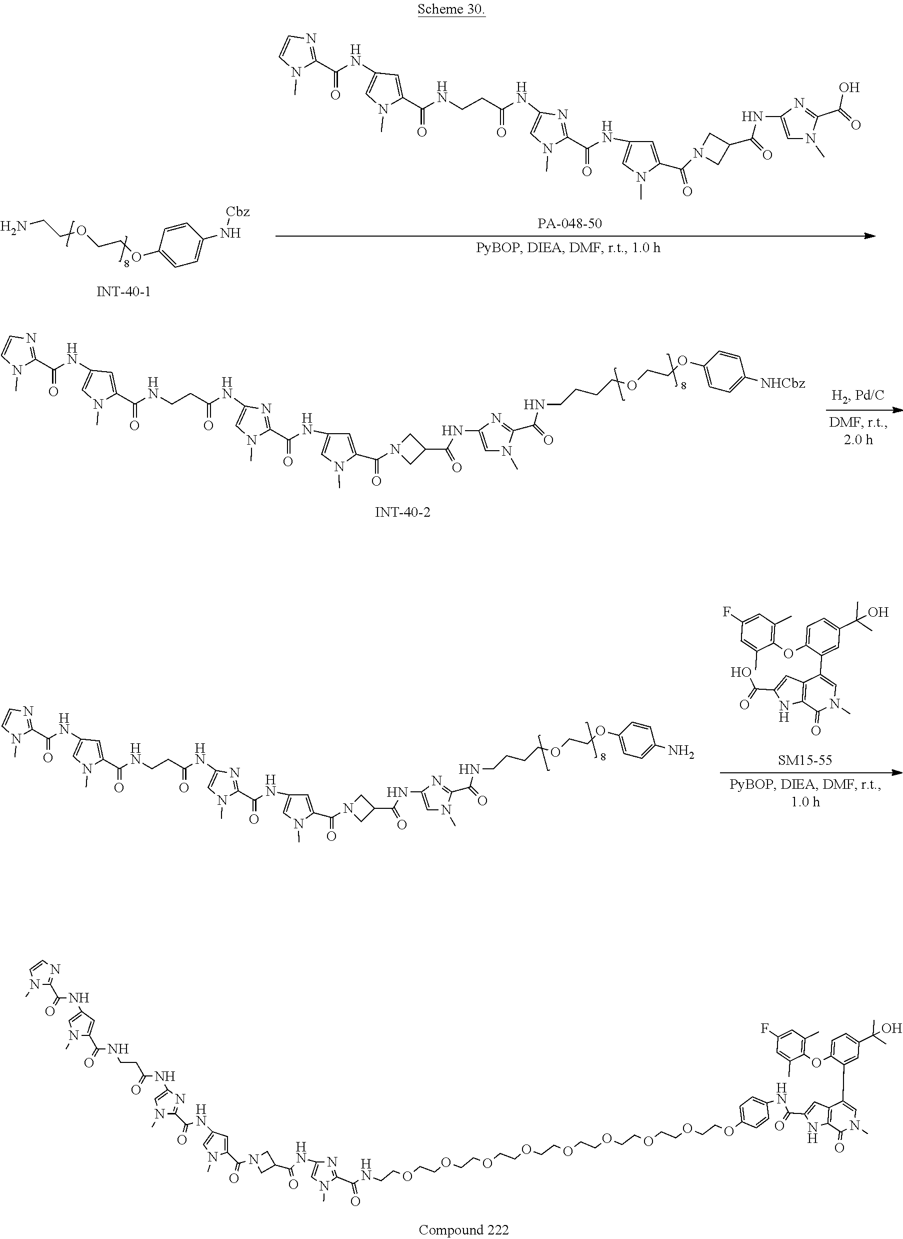

### Step 1: Synthesis of benzyl N-4-[(26-{[1-methyl-4-(1-[1-methyl-4-[1-methyl-4-(3-{[1-methyl-4-(1-methylimidazole-2-amido)pyrrol-2-yl]formamido}propanamido)imidazole-2-amido]pyrrole-2-carbonyl}azetidine-3-amido)imidazol-2-yl]formamido}-3,6,9,12,15,18,21,24-octaoxahexacosan-1-yl)oxy]phenyl}carbamate

The procedure was the same as methyl 1-methyl-4-(1-methyl-4-{1-methyl-4-[1-methyl-4-(1-methylimidazole-2-amido)pyrrole-2-amido]pyrrole-2-amido}imidazole-2-amido)pyrrole-2-carboxylate. 140.00 mg of 1-methyl-4-(1-{1-methyl-4-[1-methyl-4-(3-{[1-methyl-4-(1-methylimidazole-2-amido)pyrrol-2-yl]formamido}propanamido)imidazole-2-amido]pyrrole-2-carbonyl}azetidine-3-amido)imidazole-2-carboxylic acid was used, 137.00 mg of desired product was obtained as white solid (52.04% yield). LC/MS: mass calcd. for C₆₆H₈₆N₁₆O₁₈: 1390.63, found: 696.95 [M/2+H]⁺.

### Step 2: Synthesis of N-[26-(4-aminophenoxy)-3,6,9,12,15,18,21,24-octaoxahexacosan-1-yl]-1-methyl-4-(1-[1-methyl-4-[1-methyl-4-(3-{[1-methyl-4-(1-methylimidazole-2-amido)pyrrol-2-yl]formamido}propanamido)imidazole-2-amido)pyrrole-2-carbonyl}azetidine-3-amido)imidazole-2-carboxamide

The procedure was the same as 9H-fluoren-9-ylmethyl N-[2-({2-[(5-{[2-({2-[(2-{[26-(4-aminophenoxy)-3,6,9,12,15,18,21,24-octaoxahexacosan-1-yl]carbamoyl}ethyl)carbamoyl]-1-methylimidazol-4-yl}carbamoyl)ethyl]carbamoyl}-1-methylpyrrol-3-yl)carbamoyl]-1-methylimidazol-4-yl}carbamoyl)ethyl]carbamate (Example 35 Step 6). 137.00 mg of benzyl N-{4-[(26-{[1-methyl-4-(1-{1-methyl-4-[1-methyl-4-(3-{[1-methyl-4-(1-methylimidazole-2-amido)pyrrol-2-yl]formamido}propanamido)imidazole-2-amido]pyrrole-2-carbonyl}azetidine-3-amido)imidazol-2-yl]formamido}-3,6,9,12,15,18,21,24-octaoxahexacosan-1-yl)oxy]phenyl}carbamate was used, 90.00 mg crude of desired product was obtained as yellow oil. LC/MS: mass calcd. for C₅₈H₈N₁₆O₁₆: 1256.59, found: 629.80 [M/2+H]⁺.

### Step 3: Synthesis of Compound 222

The procedure was the same as N-(5-{[2-({2-[(2-{[26-(4-{1-ethyl-4-[2-(4-fluoro-2,6-dimethylphenoxy)-5-(2-hydroxypropan-2-yl)phenyl]-6-methyl-7-oxopyrrolo[2,3-c]pyridine-2-amido}phenoxy)-3,6,9,12,15,18,21,24-octaoxahexacosan-1-yl]carbamoyl}ethyl)carbamoyl]-1-methylimidazol-4-yl}carbamoyl)ethyl]carbamoyl}-1-methylpyrrol-3-yl)-1-methyl-4-(3-{[1-methyl-4-(1-methylimidazole-2-amido)pyrrol-2-yl]formamido}propanamido)imidazole-2-carboxamide (Example 20). 70.00 mg of N-[26-(4-aminophenoxy)-3,6,9,12,15,18,21,24-octaoxahexacosan-1-yl]-1-methyl-4-(1-{1-methyl-4-[1-methyl-4-(3-{[1-methyl-4-(1-methylimidazole-2-amido)pyrrol-2-yl]formamido}propanamido)imidazole-2-amido]pyrrole-2-carbonyl}azetidine-3-amido)imidazole-2-carboxamide was used, 6.90 mg of desired product was obtained as white solid (6.94% yield). HRMS: mass calcd. For C₈₄H₁₀₃FN₁₈O₂₀: 1702.7580, found: 1703.7563 [M+H]⁺.

### Example 41. Synthesis of N-(5-{[2-({2-[(2-{[26-(4-{4-[2-(4-fluoro-2,6-dimethylphenoxy)-5-(2-hydroxypropan-2-yl)phenyl]-6-methyl-7-oxo-1H-pyrrolo[2,3-c]pyridine-2-amido}phenoxy)-3,6,9,12,15,18,21,24-octaoxahexacosan-1-yl]carbamoyl]ethyl)carbamoyl]-1-methylimidazol-4-yl]carbamoyl)ethyl]carbamoyl]-1H-pyrrol-3-yl)-1-methyl-4-(3-{[4-(1-methylimidazole-2-amido)-1H-pyrrol-2-yl]formamido]propanamido)imidazole-2-carboxamide (Compound 224)

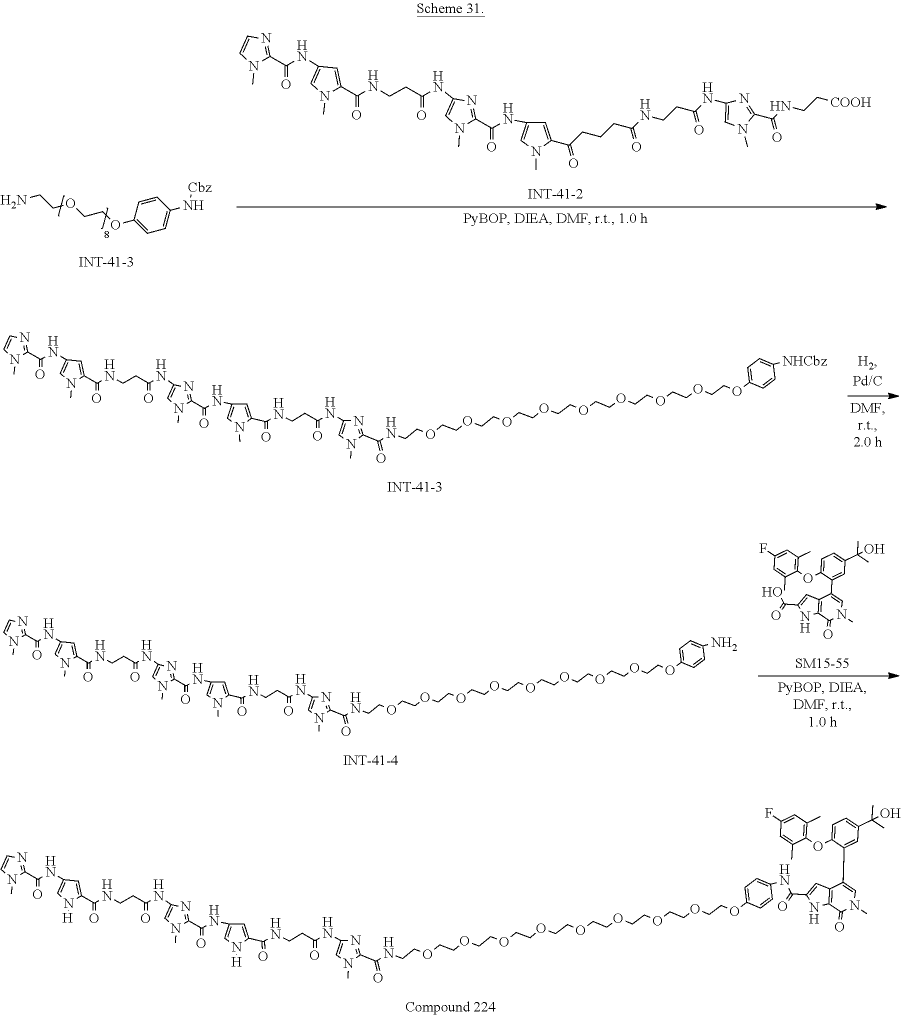

### Step 1: Synthesis of benzyl N-[4-([26-[3-({I-methyl-4-[3-([4-[I-methyl-4-(3-{[4-(1-methylimidazole-2-amido)-1H-pyrrol-2-yl]formamido}propanamido)imidazole-2-amido]-1H-pyrrol-2-yl}formamido)propanamido]imidazol-2-yl}formamido)propanamido]-3,6,9,12,15,18,21,24-octaoxahexacosan-1-yl}oxy)phenyl]carbamate

The procedure was the same as methyl 4-(4-{4-[(tert-butoxycarbonyl)amino]-1-methylpyrrole-2-amido}-1-methylimidazole-2-amido)-1-methylpyrrole-2-carboxylate (Example 2 Step 3). 265.00 mg of 3-({1-methyl-4-[3-({4-[1-methyl-4-(3-{[4-(1-methylimidazole-2-amido)-1H-pyrrol-2-yl]formamido}propanamido)imidazole-2-amido]-1H-pyrrol-2-yl}formamido)propanamido]imidazol-2-yl}formamido)propanoic acid was used, 383.50 mg of desired product was obtained as brown solid (60.71% yield). LC/MS: mass calcd. for C₆₆H₈₇N₁₇O₁₉: 1421.64, found: 712.40 [M/2+H]⁺.

### Step 2: Synthesis of N-(5-{[2-({2-[(2-{[26-(4-aminophenoxy)-3,6,9,12,15,18,21,24-octaoxahexacosan-1-yl]carbamoyl}ethyl)carbamoyl]-1-methylimidazol-4-yl}carbamoyl)ethyl]carbamoyl}-1H-pyrrol-3-yl)-1-methyl-4-(3-{[4-(1-methylimidazole-2-amido)-1H-pyrrol-2-yl]formamido}propanamido)imidazole-2-carboxamide

The procedure was the same as 9H-fluoren-9-ylmethyl N-[2-({2-[(5-{[2-({2-[(2-{[26-(4-aminophenoxy)-3,6,9,12,15,18,21,24-octaoxahexacosan-1-yl]carbamoyl}ethyl)carbamoyl]-1-methylimidazol-4-yl}carbamoyl)ethyl]carbamoyl}-1-methylpyrrol-3-yl)carbamoyl]-1-methylimidazol-4-yl}carbamoyl)ethyl]carbamate, but the reaction time was 4.0 h. 373.50 mg of benzyl N-[4-({26-[3-({1-methyl-4-[3-({4-[1-methyl-4-(3-{[4-(1-methylimidazole-2-amido)-1H-pyrrol-2-yl]formamido}propanamido)imidazole-2-amido]-1H-pyrrol-2-yl}formamido)propanamido]imidazol-2-yl}formamido)propanamido]-3,6,9,12,15,18,21,24-octaoxahexacosan-1-yl}oxy)phenyl]carbamate was used, 326.20 mg crude of desired product was obtained as brown solid. LC/MS: mass calcd. for C₅₈H₈₁N₁₇O₁₇: 1287.60, found: 645.35 [M/2+H]⁺.

### Step 3: Synthesis of Compound 224

The procedure was the same as N-(5-{[2-({2-[(2-{[26-(4-{1-ethyl-4-[2-(4-fluoro-2,6-dimethylphenoxy)-5-(2-hydroxypropan-2-yl)phenyl]-6-methyl-7-oxopyrrolo[2,3-c]pyridine-2-amido}phenoxy)-3,6,9,12,15,18,21,24-octaoxahexacosan-1-yl]carbamoyl}ethyl)carbamoyl]-1-methylimidazol-4-yl}carbamoyl)ethyl]carbamoyl}-1-methylpyrrol-3-yl)-1-methyl-4-(3-{[1-methyl-4-(1-methylimidazole-2-amido)pyrrol-2-yl]formamido}propanamido)imidazole-2-carboxamide. 150.00 mg of N-(5-{[2-({2-[(2-{[26-(4-aminophenoxy)-3,6,9,12,15,18,21,24-octaoxahexacosan-1-yl]carbamoyl}ethyl)carbamoyl]-1-methylimidazol-4-yl}carbamoyl)ethyl]carbamoyl}-1H-pyrrol-3-yl)-1-methyl-4-(3-{[4-(1-methylimidazole-2-amido)-1H-pyrrol-2-yl]formamido}propanamido)imidazole-2-carboxamide was used, 31.00 mg of desired product was obtained as white solid (14.90% yield). HRMS: mass calcd. For C₈₄H₁₀₄FN₁₉O₂₁: 1733.7638, found: 1734.7781 [M+H]⁺.

### Example 42. Synthesis of N-(5-{[2-({2-[(2-{[29-(4-{4-[2-(4-fluoro-2,6-dimethylphenoxy)-5-(2-hydroxypropan-2-yl)phenyl]-6-methyl-7-oxo-1H-pyrrolo[2,3-c]pyridine-2-amido}phenoxy)-3,6,9,12,15,18,21,24,27-nonaoxanonacosan-1-yl]carbamoyl}ethyl)carbamoyl]-1-methylimidazol-4-yl]carbamoyl)ethyl]carbamoyl]-1-methylpyrrol-3-yl)-1-methyl-4-(3-{[1-methyl-4-(1-methylimidazole-2-amido)pyrrol-2-yl]formamido]propanamido)imidazole-2-carboxamide (Compound 195)

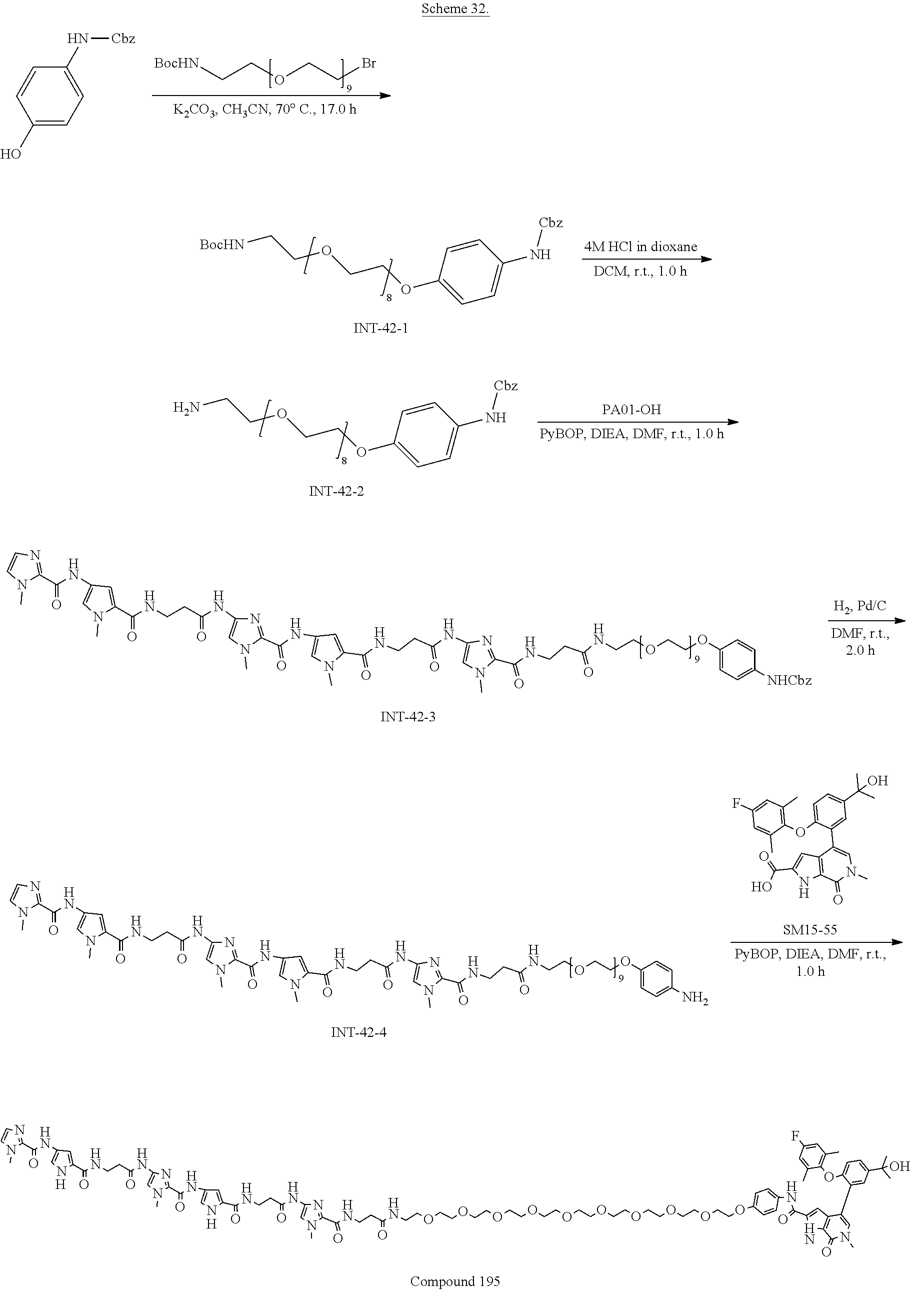

### Step 1: Synthesis of benzyl N-[4-({29-[(tert-butoxycarbonyl)amino]-3,6,9,12,15,18,21,24,27-nonaoxanonacosan-1-yl}oxy)phenyl]carbamate

The procedure was the same as ethyl 1-(5-bromopentyl)-4-[(tert-butoxycarbonyl)amino]pyrrole-2-carboxylate, but the reaction time was 17.0 h. 100.00 mg of benzyl N-(4-hydroxyphenyl)carbamate was used, 180.00 mg of desired product was obtained as yellow oil (55.93% yield). LC/MS: mass calcd. for C₃₉H₆₂N₂O₁₄: 782.42, found: 800.55 [M+H₂O]⁺.

### Step 2: Synthesis of benzyl N-{4-[(29-amino-3,6,9,12,15,18,21,24,27-nonaoxanonacosan-1-yl)oxy]phenyl}carbamate

The procedure was the same as methyl 4-[4-(3-aminopropanamido)-1-methylimidazole-2-amido]-1-methylpyrrole-2-carboxylate hydrochloride, but the reaction solvent was 4M HCl in dioxane/DCM (1:1) and the reaction time was 1.0 h. 170.00 mg of benzyl N-[4-({29-[(tert-butoxycarbonyl)amino]-3,6,9,12,15,18,21,24,27-nonaoxanonacosan-1-yl}oxy)phenyl]carbamate was used, 170.00 mg crude of desired product was obtained as colorless oil. LC/MS: mass calcd. for C₃₄H₅₄N₂O₁₂: 682.37, found: 683.35 [M+H]⁺.

### Step 3: Synthesis of benzyl N-[4-({29-[3-({1-methyl-4-[3-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)propanamido]imidazol-2-yl}formamido)propanamido]-3,6,9,12,15,18,21,24,27-nonaoxanonacosan-1-yl}oxy)phenyl]carbamate

The procedure was the same as ethyl 1-methyl-4-[3-methyl-3-({1-methyl-4-[1-methyl-4-(3-{[1-methyl-4-(1-methylimidazole-2-amido)pyrrol-2-yl] formamido}propanamido)imidazole-2-amido]pyrrol-2-yl}formamido)butanamido]imidazole-2-carboxylate. 170.00 mg of benzyl N-{4-[(29-amino-3,6,9,12,15,18,21,24,27-nonaoxanonacosan-1-yl)oxy]phenyl}carbamate was used, 320.00 mg of crude product was obtained as yellow solid (85.99% yield). LC/MS: mass calcd. for C₇₀H₉₅N₁₇O₂₀: 1493.69, found: 1494.90 [M+H]⁺.

### Step 4: Synthesis of N-(5-{[2-({2-[(2-{[29-(4-aminophenoxy)-3,6,9,12,15,18,21,24,27-nonaoxanonacosan-1-yl]carbamoyl}ethyl)carbamoyl]-1-methylimidazol-4-yl}carbamoyl)ethyl]carbamoyl}-1-methylpyrrol-3-yl)-1-methyl-4-(3-{[1-methyl-4-(1-methylimidazole-2-amido)pyrrol-2-yl]formamido}propanamido)imidazole-2-carboxamide

The procedure was the same as N-(5-{[2-({2-[(2-{[26-(4-aminophenoxy)-3,6,9,12,15,18,21,24-octaoxahexacosan-1-yl]carbamoyl}ethyl)carbamoyl]-1-methylimidazol-4-yl}carbamoyl)ethyl]carbamoyl}-1-methylpyrrol-3-yl)-1-methyl-4-(3-{[1-methyl-4-(1-methylimidazole-2-amido)pyrrol-2-yl]formamido}propanamido)imidazole-2-carboxamide, but the reaction time was 4.0 h. 300.00 mg of benzyl N-[4-({29-[3-({1-methyl-4-[3-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)propanamido]imidazol-2-yl}formamido)propanamido]-3,6,9,12,15,18,21,24,27-nonaoxanonacosan-1-yl}oxy)phenyl]carbamate was used, 230.00 mg of desired product was obtained as white solid (84.23% yield). LC/MS: mass calcd. for C₆₂H₈₉N₁₇O₁₃: 1359.66, found: 1360.90 [M+H]⁺.

### Step 5: Synthesis of Compound 195

The procedure was the same as N-(5-{[2-({2-[(2-{[26-(4-{1-ethyl-4-[2-(4-fluoro-2,6-dimethylphenoxy)-5-(2-hydroxypropan-2-yl)phenyl]-6-methyl-7-oxopyrrolo[2,3-c]pyridine-2-amido}phenoxy)-3,6,9,12,15,18,21,24-octaoxahexacosan-1-yl]carbamoyl}ethyl)carbamoyl]-1-methylimidazol-4-yl}carbamoyl)ethyl]carbamoyl}-1-methylpyrrol-3-yl)-1-methyl-4-(3-{[1-methyl-4-(1-methylimidazole-2-amido)pyrrol-2-yl]formamido}propanamido)imidazole-2-carboxamide (Example 20). 210.00 mg of N-(5-{[2-({2-[(2-{[29-(4-aminophenoxy)-3,6,9,12,15,18,21,24,27-nonaoxanonacosan-1-yl]carbamoyl}ethyl)carbamoyl]-1-methylimidazol-4-yl}carbamoyl)ethyl]carbamoyl}-1-methylpyrrol-3-yl)-1-methyl-4-(3-{[1-methyl-4-(1-methylimidazole-2-amido)pyrrol-2-yl]formamido}propanamido)imidazole-2-carboxamide was used, 32.50 mg of desired product was obtained as white solid (11.42% yield). HRMS: mass calcd. for C₈₈H₁₁₂FN₁₉O₂₂: 1805.8213, found: 1806.8270 [M+H]⁺.

### Example 43. Synthesis of N-(5-{[2-({2-[(2-{[23-(4-{4-[2-(4-fluoro-2,6-dimethylphenoxy)-5-(2-hydroxypropan-2-yl)phenyl]-6-methyl-7-oxo-1H-pyrrolo[2,3-c]pyridine-2-amido}phenoxy)-3,6,9,12,15,18,21-heptaoxatricosan-1-yl]carbamoyl]ethyl)carbamoyl]-1-methylimidazol-4-yl]carbamoyl)ethyl]carbamoyl]-1-methylpyrrol-3-yl)-1-methyl-4-(3-{[1-methyl-4-(1-methylimidazole-2-amido)pyrrol-2-yl]formamido}propanamido)imidazole-2-carboxamide (Compound 205)

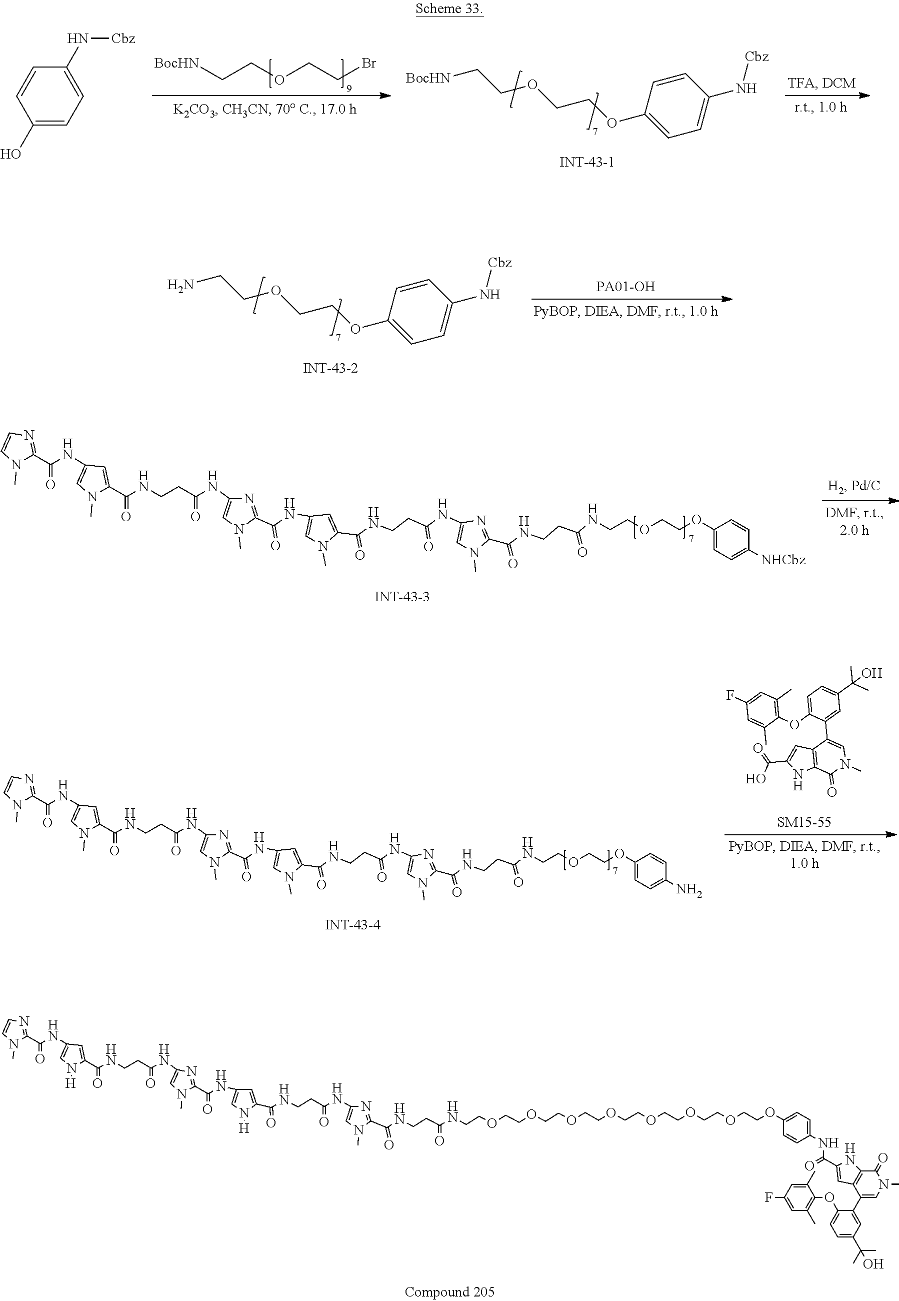

### Step 1: Synthesis of benzyl N-[4-({23-[(tert-butoxycarbonyl)amino]-3,6,9,12,15,18,21-heptaoxatricosan-1-yl}oxy)phenyl]carbamate

The procedure was the same as ethyl 1-(5-bromopentyl)-4-[(tert-butoxycarbonyl)amino]pyrrole-2-carboxylate, but the reaction time was 17.0 h. 300.00 mg of benzyl N-(4-hydroxyphenyl)carbamate was used, 760.00 mg of benzyl N-[4-({23-[(tert-butoxycarbonyl)amino]-3,6,9,12,15,18,21-heptaoxatricosan-1-yl}oxy)phenyl]carbamate was obtained as yellow oil (77.16% yield). LC/MS: mass calcd. for C₃₅H₅₄N₂O₁₂: 694.37, found: 695.55[M+H]⁺.

### Step 2: Synthesis of benzyl N-{4-[(23-amino-3,6,9,12,15,18,21-heptaoxatricosan-1-yl)oxy]phenyl}carbamate

The procedure was the same as methyl 4-[4-(4-amino-1-methylpyrrole-2-amido)-1-methylimidazole-2-amido]-1-methylpyrrole-2-carboxylate (Example 2 Step 4). 760.00 mg of benzyl N-[4-({23-[(tert-butoxycarbonyl)amino]-3,6,9,12,15,18,21-heptaoxatricosan-1-yl}oxy)phenyl]carbamate was used, 760.00 mg crude of benzyl N-{4-[(23-amino-3,6,9,12,15,18,21-heptaoxatricosan-1-yl)oxy]phenyl}carbamate was obtained as yellow oil. LC/MS: mass calcd. for C₃₀H₄₆N₂O₁₀: 594.31, found: 595.45[M+H]⁺.

### Step 3: Synthesis of benzyl N-[4-({23-[3-({1-methyl-4-[3-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)propanamido]imidazol-2-yl}formamido)propanamido]-3,6,9,12,15,18,21-heptaoxatricosan-1-yl}oxy)phenyl]carbamate

The procedure was the same as ethyl 1-methyl-4-[3-methyl-3-({1-methyl-4-[1-methyl-4-(3-{[1-methyl-4-(1-methylimidazole-2-amido)pyrrol-2-yl] formamido}propanamido)imidazole-2-amido]pyrrol-2-yl}formamido)butanamido]imidazole-2-carboxylate. 650.00 mg of benzyl N-{4-[(23-amino-3,6,9,12,15,18,21-heptaoxatricosan-1-yl)oxy]phenyl}carbamate was used, 870.00 mg of desired product was obtained as yellow solid (45.27% yield). LC/MS: mass calcd. for C₆₆H₈₇N₁₇O₁₃: 1405.64, found: 704.35 [M/2+H]⁺.

### Step 4: Synthesis of N-(5-{[2-({2-[(2-{[23-(4-aminophenoxy)-3,6,9,12,15,18,21-heptaoxatricosan-1-yl]carbamoyl}ethyl)carbamoyl]-1-methylimidazol-4-yl}carbamoyl)ethyl]carbamoyl}-1-methylpyrrol-3-yl)-1-methyl-4-(3-{[1-methyl-4-(1-methylimidazole-2-amido)pyrrol-2-yl]formamido}propanamido)imidazole-2-carboxamide

The procedure was the same as N-(5-{[2-({2-[(2-{[26-(4-aminophenoxy)-3,6,9,12,15,18,21,24-octaoxahexacosan-1-yl]carbamoyl}ethyl)carbamoyl]-1-methylimidazol-4-yl}carbamoyl)ethyl]carbamoyl}-1-methylpyrrol-3-yl)-1-methyl-4-(3-{[1-methyl-4-(1-methylimidazole-2-amido)pyrrol-2-yl]formamido}propanamido)imidazole-2-carboxamide (INT-216-5). 820.00 mg of benzyl N-[4-({23-[3-({1-methyl-4-[3-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)propanamido]imidazol-2-yl}formamido)propanamido]-3,6,9,12,15,18,21-heptaoxatricosan-1-yl}oxy)phenyl]carbamate was used, 650.00 mg crude of desired product was obtained as yellow solid. LC/MS: mass calcd. for C₅₈H₈₁N₁₇O₁₆: 1271.60, found: 637.20 [M/2+H]⁺.

### Step 5: Synthesis of Compound 205

Into a 50 mL flask was added 4-[2-(4-fluoro-2,6-dimethylphenoxy)-5-(2-hydroxypropan-2-yl)phenyl]-6-methyl-7-oxo-1H-pyrrolo[2,3-c]pyridine-2-carboxylic acid (166.00 mg, 0.36 mmol, 1.01 equiv), DMF (8.00 mL), N-(5-{[2-({2-[(2-{[23-(4-aminophenoxy)-3,6,9,12,15,18,21-heptaoxatricosan-1-yl]carbamoyl}ethyl)carbamoyl]-1-methylimidazol-4-yl}carbamoyl)ethyl]carbamoyl}-1-methylpyrrol-3-yl)-1-methyl-4-(3-{[1-methyl-4-(1-methylimidazole-2-amido)pyrrol-2-yl]formamido}propanamido)imidazole-2-carboxamide (450.00 mg, 0.35 mmol, 1.00 equiv), PyBOP (240.00 mg, 0.46 mmol, 1.30 equiv), the mixture was stirred at room temperature for 5.0 mins, then DIEA (184.00 mg, 1.42 mmol, 4.03 equiv) was added, the reaction was stirred at room temperature for 1.0 h. The reaction was poured into ice water (30 mL). The precipitated solids were collected by filtration and washed with water (3×5 mL), dried under vacuum. The residue was purified by silica gel column chromatography, eluted with CH₂C₁₂/MeOH (8:1). The fractions were combined and concentrated. The residue was purified by reverse flash chromatography with the following conditions: column, silica gel; mobile phase, MeCN in water (0.05% TFA), 10% to 50% gradient in 30.0 min; detector, UV 254 nm. The fractions were combined and lyophilized directly. This resulted in N-(5-{[2-({2-[(2-{[23-(4-{4-[2-(4-fluoro-2,6-dimethylphenoxy)-5-(2-hydroxypropan-2-yl)phenyl]-6-methyl-7-oxo-1H-pyrrolo[2,3-c]pyridine-2-amido}phenoxy)-3,6,9,12,15,18,21-heptaoxatricosan-1-yl]carbamoyl}ethyl)carbamoyl]-1-methylimidazol-4-yl}carbamoyl)ethyl]carbamoyl}-1-methylpyrrol-3-yl)-1-methyl-4-(3-{[1-methyl-4-(1-methylimidazole-2-amido)pyrrol-2-yl]formamido}propanamido)imidazole-2-carboxamide (215.10 mg, 34.80% yield) as white solid. HRMS: mass calcd. For C₈₄H₁₀₄FN₁₉O₂₀: 1717.7689, found: 1718.7775 [M+H]⁺.

### Example 44. Synthesis of 5-{4-[2-(4-fluoro-2,6-dimethylphenoxy)-5-(2-hydroxypropan-2-yl)phenyl]-6-methyl-7-oxo-1H-pyrrolo[2,3-c]pyridine-2-amido]-2-({26-[3-({1-methyl-4-[3-({1-methyl-4-[1-methyl-4-(3-{[1-methyl-4-(1-methylimidazole-2-amido)pyrrol-2-yl]formamido}propanamido)imidazole-2-amido)pyrrol-2-yl}formamido)propanamido]imidazol-2-yl]formamido)propanamido]-3,6,9,12,15,18,21,24-octaoxahexacosan-1-yl]oxy)phenyl 4-[2-(4-fluoro-2,6-dimethylphenoxy)-5-(2-hydroxypropan-2-yl)phenyl]-6-methyl-7-oxo-1H-pyrrolo[2,3-c]pyridine-2-carboxylate (Compound 240)

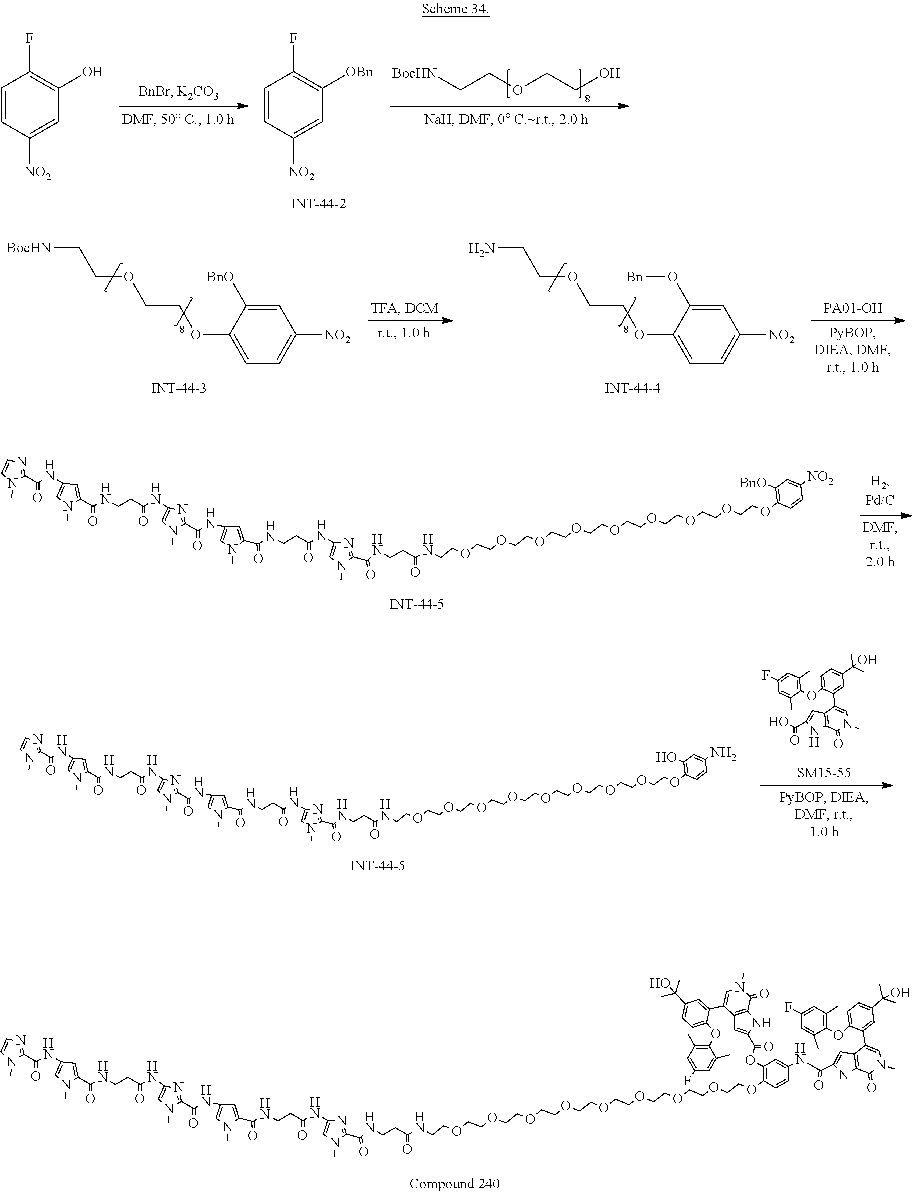

### Step 1: Synthesis of 2-(benzyloxy)-1-fluoro-4-nitrobenzene

To a stirred solution of 2-fluoro-5-nitrophenol (1.00 g, 6.365 mmol, 1.00 equiv) in DMF (15.00 mL) was added benzyl bromide (1.63 g, 9.547 mmol, 1.50 equiv) and K2CO3 (2.64 g, 19.095 mmol, 3.00 equiv). The resulting mixture was stirred at 50 degrees C. for 1.0 h. The reaction mixture was poured into ice-water (50 mL), extracted with EA (3×80 mL). The organic phases were combined and washed with H₂O (50 mL) and NaCl (50 mL), dried over anhydrous Na2SO4. The solid was filtered out and the filtrate was concentrated. The residue was purified by silica gel column chromatography (0-10% EA/PE) to afford 2-(benzyloxy)-1-fluoro-4-nitrobenzene (1.50 g, 95.32%) as yellow solid. LC/MS: mass calcd. For C₁₃H₁₀FNO₃: 247.06, found: 222.15 [M+H]⁺.

### Step 2: Synthesis of 2-(benzyloxy)-1-fluoro-4-nitrobenzene

The procedure was the same as 2-(benzyloxy)-5-nitrophenol, but the reaction temperature was 0 degrees C. to room temperature and the reaction time was 2.0 h. 100.00 mg of 2-(benzyloxy)-1-fluoro-4-nitrobenzene was used, 290.00 mg of tert-butyl N-{26-[2-(benzyloxy)-4-nitrophenoxy]-3,6,9,12,15,18,21,24-octaoxahexacosan-1-yl}carbamate was obtained as yellow oil (96.78% yield). LC/MS: mass calcd. for C₃₆H₅₆N₂O₁₄: 740.37, found: 741.50 [M+H]⁺.

### Step 3: Synthesis of 26-[2-(benzyloxy)-4-nitrophenoxy]-3,6,9,12,15,18,21,24-octaoxahexacosan-1-amine

The procedure was the same as methyl 4-[4-(4-amino-1-methylpyrrole-2-amido)-1-methylimidazole-2-amido]-1-methylpyrrole-2-carboxylate (Example 2 Step 3). 240.00 mg of tert-butyl N-{26-[2-(benzyloxy)-4-nitrophenoxy]-3,6,9,12,15,18,21,24-octaoxahexacosan-1-yl}carbamate was used, 240.00 mg crude of 26-[2-(benzyloxy)-4-nitrophenoxy]-3,6,9,12,15,18,21,24-octaoxahexacosan-1-amine was obtained as yellow oil. LC/MS: mass calcd. for C₃₁H₄₈N₂O₁₂: 640.32, found: 641.55 [M+H]⁺.

### Step 4: Synthesis of N-[5-({2-[(2-{[2-({26-[2-(benzyloxy)-4-nitrophenoxy]-3,6,9,12,15,18,21,24-octaoxahexacosan-1-yl}carbamoyl)ethyl]carbamoyl}-1-methylimidazol-4-yl)carbamoyl]ethyl}carbamoyl)-1-methylpyrrol-3-yl]-1-methyl-4-(3-{[I-methyl-4-(1-methylimidazole-2-amido)pyrrol-2-yl]formamido}propanamido)imidazole-2-carboxamide

The procedure was the same as methyl 4-(4-{4-[(tert-butoxycarbonyl)amino]-1-methylpyrrole-2-amido}-1-methylimidazole-2-amido)-1-methylpyrrole-2-carboxylate (Example 2 Step 3). 240.00 mg of 26-[2-(benzyloxy)-4-nitrophenoxy]-3,6,9,12,15,18,21,24-octaoxahexacosan-1-amine was used, 500.00 mg of N-[5-({2-[(2-{[2-({26-[2-(benzyloxy)-4-nitrophenoxy]-3,6,9,12,15,18,21,24-octaoxahexacosan-1-yl}carbamoyl)ethyl]carbamoyl}-1-methylimidazol-4-yl)carbamoyl]ethyl}carbamoyl)-1-methylpyrrol-3-yl]-1-methyl-4-(3-{[1-methyl-4-(1-methylimidazole-2-amido)pyrrol-2-yl]formamido}propanamido)imidazole-2-carboxamide was obtained as yellow solid (91.90% yield). LC/MS: mass calcd. for C₆₇H₈₉N₁₇O₂₀: 1451.64, found: 727.45 [M/2+H]⁺.

### Step 5: Synthesis of N-(5-{[2-({2-[(2-{[26-(4-amino-2-hydroxyphenoxy)-3,6,9,12,15,18,21,24-octaoxahexacosan-1-yl]carbamoyl}ethyl)carbamoyl]-1-methylimidazol-4-yl}carbamoyl)ethyl]carbamoyl}-1-methylpyrrol-3-yl)-1-methyl-4-(3-{[I-methyl-4-(1-methylimidazole-2-amido)pyrrol-2-yl]formamido}propanamido)imidazole-2-carboxamide

The procedure was the same as 9H-fluoren-9-ylmethyl N-[2-({2-[(5-{[2-({2-[(2-{[26-(4-aminophenoxy)-3,6,9,12,15,18,21,24-octaoxahexacosan-1-yl]carbamoyl}ethyl)carbamoyl]-1-methylimidazol-4-yl}carbamoyl)ethyl]carbamoyl}-1-methylpyrrol-3-yl)carbamoyl]-1-methylimidazol-4-yl}carbamoyl)ethyl]carbamate, but the reaction time was 3.0 h. 250.00 mg of N-[5-({2-[(2-{[2-({26-[2-(benzyloxy)-4-nitrophenoxy]-3,6,9,12,15,18,21,24-octaoxahexacosan-1-yl}carbamoyl)ethyl]carbamoyl}-1-methylimidazol-4-yl)carbamoyl]ethyl}carbamoyl)-1-methylpyrrol-3-yl]-1-methyl-4-(3-{[1-methyl-4-(1-methylimidazole-2-amido)pyrrol-2-yl]formamido}propanamido)imidazole-2-carboxamide was used, 250.00 mg crude of N-(5-{[2-({2-[(2-{[26-(4-amino-2-hydroxyphenoxy)-3,6,9,12,15,18,21,24-octaoxahexacosan-1-yl]carbamoyl}ethyl)carbamoyl]-1-methylimidazol-4-yl}carbamoyl)ethyl]carbamoyl}-1-methylpyrrol-3-yl)-1-methyl-4-(3-{[1-methyl-4-(1-methylimidazole-2-amido)pyrrol-2-yl]formamido}propanamido)imidazole-2-carboxamide was obtained as brown oil. LC/MS: mass calcd. for C₆₀H₈₅N₁₇O₁₈: 1331.62, found: 667.30 [M/2+H]⁺.

### Step 6: Synthesis of Compound 240

The procedure was the same as methyl 1-methyl-4-(1-methyl-4-{1-methyl-4-[1-methyl-4-(1-methylimidazole-2-amido)pyrrole-2-amido]pyrrole-2-amido}imidazole-2-amido)pyrrole-2-carboxylate (Example 2 Step 5). 130.00 mg of N-(5-{[2-({2-[(2-{[26-(4-amino-2-hydroxyphenoxy)-3,6,9,12,15,18,21,24-octaoxahexacosan-1-yl]carbamoyl}ethyl)carbamoyl]-1-methylimidazol-4-yl}carbamoyl)ethyl]carbamoyl}-1-methylpyrrol-3-yl)-1-methyl-4-(3-{[1-methyl-4-(1-methylimidazole-2-amido)pyrrol-2-yl]formamido}propanamido)imidazole-2-carboxamide was used, 70.00 mg of 5-{4-[2-(4-fluoro-2,6-dimethylphenoxy)-5-(2-hydroxypropan-2-yl)phenyl]-6-methyl-7-oxo-1H-pyrrolo[2,3-c]pyridine-2-amido}-2-({26-[3-({1-methyl-4-[3-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)propanamido]imidazol-2-yl}formamido)propanamido]-3,6,9,12,15,18,21,24-octaoxahexacosan-1-yl}oxy)phenyl 4-[2-(4-fluoro-2,6-dimethylphenoxy)-5-(2-hydroxypropan-2-yl)phenyl]-6-methyl-7-oxo-1H-pyrrolo[2,3-c]pyridine-2-carboxylate was obtained as white solid (32.24% yield).

LC/MS: mass calcd. For C₁₁₂H₁₃₁F₂N₂₁O₂₆: 2223.95, found: 1113.45 [M/2+H]⁺.

### Example 45. Synthesis of N-({1-[23-(4-{14-[2-(4-fluoro-2,6-dimethylphenoxy)-5-(2-hydroxypropan-2-yl)phenyl]-6-methyl-7-oxo-1H-pyrrolo[2,3-c]pyridine-2-amido}phenoxy)-3,6,9,12,15,18,21-heptaoxatricosan-1-yl]-1,2,3-triazol-4-yl]methyl)-1-methyl-4-[3-({1-methyl-4-[1-methyl-4-(3-{[1-methyl-4-(1-methylimidazole-2-amido)pyrrol-2-yl]formamido]propanamido)imidazole-2-amido]pyrrol-2-yl]formamido)propanamido]imidazole-2-carboxamide (Compound 217)

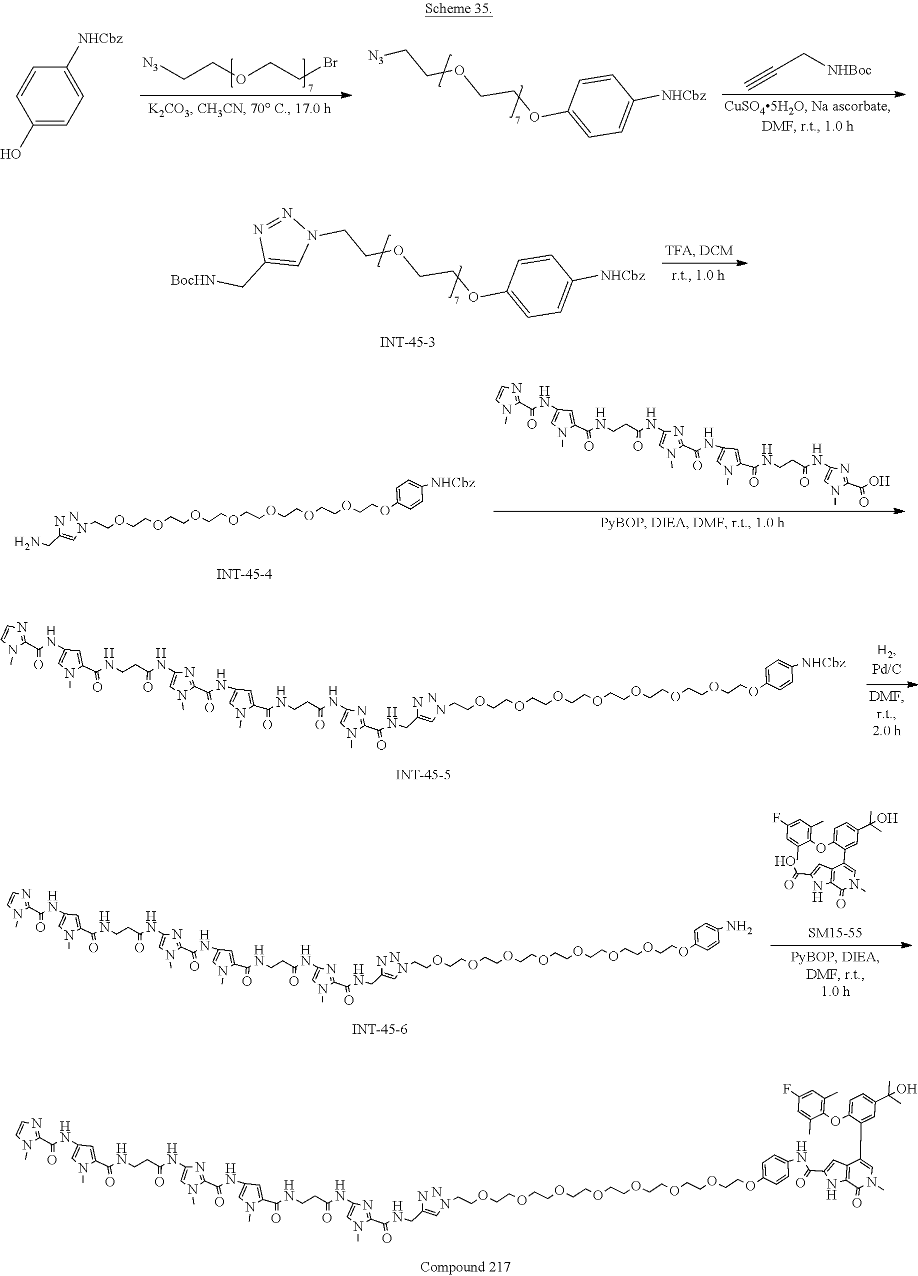

### Step 1: Synthesis of benzyl N-{4-[(23-azido-3,6,9,12,15,18,21-heptaoxatricosan-1-yl)oxy]phenyl}carbamate

The procedure was the same as ethyl 1-(5-bromopentyl)-4-[(tert-butoxycarbonyl)amino]pyrrole-2-carboxylate, but the reaction time was 17.0 h. 200.00 mg of benzyl N-(4-hydroxyphenyl)carbamate was used, 280.00 mg of benzyl N-{4-[(23-azido-3,6,9,12,15,18,21-heptaoxatricosan-1-yl)oxy]phenyl}carbamate was obtained as light brown oil (54.87% yield). LC/MS: mass calcd. For C₃₀H₄₄N₄O₁₀: 620.30, found: 621.55 [M+H]⁺.

### Step 2: Synthesis of benzyl N-(4-{[23-(4-{[(tert-butoxycarbonyl)amino]methyl}-1,2,3-triazol-1-yl)-3,6,9,12,15,18,21-heptaoxatricosan-1-yl]oxy}phenyl)carbamate

To a stirred solution of tert-butyl N-(prop-2-yn-1-yl)carbamate (70.01 mg, 0.451 mmol, 1.00 equiv) in DMF (3.00 mL), benzyl N-{4-[(23-azido-3,6,9,12,15,18,21-heptaoxatricosan-1-yl)oxy]phenyl}carbamate (280.00 mg, 0.451 mmol, 1.00 equiv), sodium ascorbate (44.91 mg, 0.226 mmol, 0.50 equiv) and CuSO4·5H₂O (56.32 mg, 0.226 mmol, 0.50 equiv) were added in turn at room temperature and the resulting mixture was stirred for 1.0 h at room temperature. The resulting mixture was poured into water (150 mL) and extracted with DCM (2×150 mL). The combined organic layers were washed with H₂O (3×100 mL), dried over anhydrous Na2SO4. After filtration, the filtrate was concentrated under reduced pressure. Then the crude product was purified by TLC-plate (DCM:MeOH=10:1) to afford benzyl N-(4-{[23-(4-{[(tert-butoxycarbonyl)amino]methyl}-1,2,3-triazol-1-yl)-3,6,9,12,15,18,21-heptaoxatricosan-1-yl]oxy}phenyl)carbamate (260.00 mg, 74.28%) as light brown oil. LC/MS: mass calcd. For C₃₈H₅₇N₅O₁₂: 775.40, found: 776.70 [M+H]⁺.

### Step 3: Synthesis of benzyl N-[4-({23-[4-(aminomethyl)-1,2,3-triazol-1-yl]-3,6,9,12,15,18,21-heptaoxatricosan-1-yl}oxy)phenyl]carbamate

The procedure was the same as methyl 4-[4-(4-amino-1-methylpyrrole-2-amido)-1-methylimidazole-2-amido]-1-methylpyrrole-2-carboxylate (Example 2 Step 4). 180.00 mg of benzyl N-(4-{[23-(4-{[(tert-butoxycarbonyl)amino]methyl}-1,2,3-triazol-1-yl)-3,6,9,12,15,18,21-heptaoxatricosan-1-yl]oxy}phenyl)carbamate was used, 180.00 mg crude of benzyl N-[4-({23-[4-(aminomethyl)-1,2,3-triazol-1-yl]-3,6,9,12,15,18,21-heptaoxatricosan-1-yl}oxy)phenyl]carbamate was obtained as light yellow oil.

LC/MS: mass calcd. For C₃₃H₄₉N₅O₁₀: 675.34, found: 676.30 [M+H]⁺.

### Step 4: Synthesis of benzyl N-{4-[(23-{4-[({1-methyl-4-[3-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)propanamido]imidazol-2-yl}formamido)methyl]-1,2,3-triazol-1-yl}-3,6,9,12,15,18,21-heptaoxatricosan-1-yl)oxy]phenyl}carbamate

The procedure was the same as ethyl 1-methyl-4-[3-methyl-3-({1-methyl-4-[1-methyl-4-(3-{[1-methyl-4-(1-methylimidazole-2-amido)pyrrol-2-yl] formamido}propanamido)imidazole-2-amido]pyrrol-2-yl}formamido)butanamido]imidazole-2-carboxylate, but the reaction time was 1.0 h. 150.00 mg of 1-methyl-4-[3-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)propanamido]imidazole-2-carboxylic acid was used, 112.00 mg of benzyl N-{4-[(23-{4-[({1-methyl-4-[3-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)propanamido]imidazol-2-yl}formamido)methyl]-1,2,3-triazol-1-yl}-3,6,9,12,15,18,21-heptaoxatricosan-1-yl)oxy]phenyl}carbamate was obtained as light yellow solid (40.00% yield). LC/MS: mass calcd. For C₆₆H₈₅N₁₉O₁₇: 1415.63, found: 709.45 [M/2+H]⁺.

### Step 5: Synthesis of N-([1-[23-(4-aminophenoxy)-3,6,9,12,15,18,21-heptaoxatricosan-1-yl]-1,2,3-triazol-4-yl}methyl)-1-methyl-4-[3-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)propanamido]imidazole-2-carboxamide

The procedure was the same as 9H-fluoren-9-ylmethyl N-[2-({2-[(5-{[2-({2-[(2-{[26-(4-aminophenoxy)-3,6,9,12,15,18,21,24-octaoxahexacosan-1-yl]carbamoyl}ethyl)carbamoyl]-1-methylimidazol-4-yl}carbamoyl)ethyl]carbamoyl}-1-methylpyrrol-3-yl)carbamoyl]-1-methylimidazol-4-yl}carbamoyl)ethyl]carbamate (Example 35 Step 6), but the reaction time was 17.0 h. 110.00 mg of benzyl N-{4-[(23-{4-[({1-methyl-4-[3-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)propanamido]imidazol-2-yl}formamido)methyl]-1,2,3-triazol-1-yl}-3,6,9,12,15,18,21-heptaoxatricosan-1-yl)oxy]phenyl}carbamate was used, 100.00 mg crude of N-({1-[23-(4-aminophenoxy)-3,6,9,12,15,18,21-heptaoxatricosan-1-yl]-1,2,3-triazol-4-yl}methyl)-1-methyl-4-[3-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)propanamido]imidazole-2-carboxamide was obtained as brown solid.

LC/MS: mass calcd. For C₅₈H₇₉N₁₉O₁₅: 1281.60, found: 1282.55 [M+H]⁺.

### Step 6: Synthesis of Compound 217

The procedure was the same as N-(5-{[2-({2-[(2-{[26-(4-{1-ethyl-4-[2-(4-fluoro-2,6-dimethylphenoxy)-5-(2-hydroxypropan-2-yl)phenyl]-6-methyl-7-oxopyrrolo[2,3-c]pyridine-2-amido}phenoxy)-3,6,9,12,15,18,21,24-octaoxahexacosan-1-yl]carbamoyl}ethyl)carbamoyl]-1-methylimidazol-4-yl}carbamoyl)ethyl]carbamoyl}-1-methylpyrrol-3-yl)-1-methyl-4-(3-{[1-methyl-4-(1-methylimidazole-2-amido)pyrrol-2-yl]formamido}propanamido)imidazole-2-carboxamide (Example 20). 100.00 mg of N-({1-[23-(4-aminophenoxy)-3,6,9,12,15,18,21-heptaoxatricosan-1-yl]-1,2,3-triazol-4-yl}methyl)-1-methyl-4-[3-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)propanamido]imidazole-2-carboxamide was used, 29.00 mg of N-({1-[23-(4-{4-[2-(4-fluoro-2,6-dimethylphenoxy)-5-(2-hydroxypropan-2-yl)phenyl]-6-methyl-7-oxo-1H-pyrrolo[2,3-c]pyridine-2-amido}phenoxy)-3,6,9,12,15,18,21-heptaoxatricosan-1-yl]-1,2,3-triazol-4-yl}methyl)-1-methyl-4-[3-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)propanamido]imidazole-2-carboxamide was obtained as white solid (21.47% yield). HRMS: mass calcd. For C₈₄H₁₀₂FN₂₁O₁₉: 1727.7644, found: 1728.7740 [M+H]⁺.

Example 46. Synthesis of N-(5-{[2-(2-[(2-{[20-(4-˜4-[2-(4-fluoro-2,6-dimethylphenoxy)-5-(2-hydroxypropan-2-yl)phenyl]-6-methyl-7-oxo-1H-pyrrolo[2,3-c]pyridine-2-amido}phenoxy)-3,6,9,12,15,18-hexaoxaicosan-1-yl]carbamoyl]ethyl)carbamoyl]-1-methylimidazol-4-yl]carbamoyl)ethyl]carbamoyl]-1-[14-(2,5,8,11-tetraoxatridecan-13-yl)-2,5,8,11-tetraoxa-14-azanonadecan-19-yl]pyrrol-3-yl)-1-methyl-4-(3-{[1-methyl-4-(1-methylimidazole-2-amido)pyrrol-2-yl]formamido}propanamido)imidazole-2-carboxamide (Compound 251)

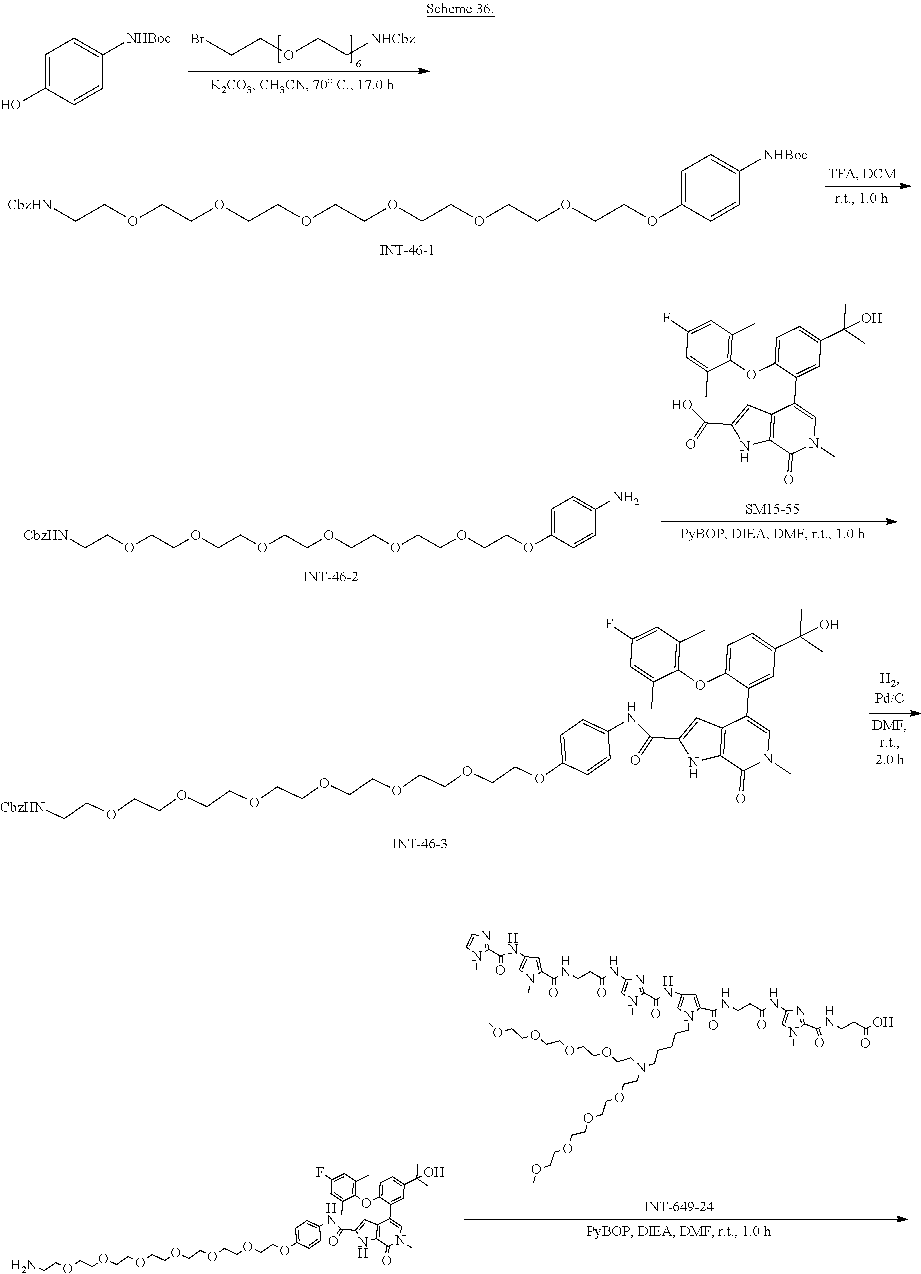

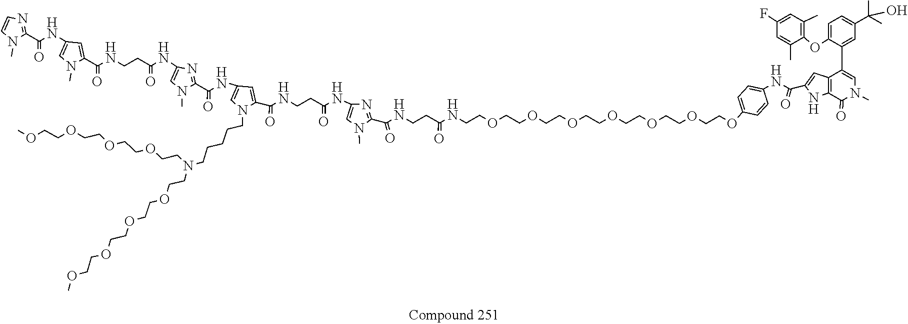 [SMILES: COCCOCCOCCOCCN(CCCCCn1cc(NC(=O)c2nc(NC(=O)CCNC(=O)c3cc(NC(=O)c4nccn4C)cn3C)cn2C)cc1C(=O)NCCC(=O)Nc1cn(C)c(C(=O)NCCC(=O)NCCOCCOCCOCCOCCOCCOCCOc2ccc(NC(=O)c3cc4c(-c5cc(C(C)(C)O)ccc5Oc5c(C)cc(F)cc5C)cn(C)c(=O)c4[nH]3)cc2)n1)CCOCCOCCOCCOC]

### Step 1: Synthesis of benzyl N-(20-[4-[(tert-butoxycarbonyl)amino)phenoxy]-3,6,9,12,15,18-hexaoxaicosan-1-yl)carbamate

The procedure was the same as ethyl 1-(5-bromopentyl)-4-[(tert-butoxycarbonyl)amino]pyrrole-2-carboxylate, but the reaction time was 17.0 h. 200.00 mg of tert-butyl N-(4-hydroxyphenyl)carbamate was used, 1.30 g of benzyl N-(20-{4-[(tert-butoxycarbonyl)amino]phenoxy}-3,6,9,12,15,18-hexaoxaicosan-1-yl)carbamate was obtained as yellow oil (crude). LC/MS: mass calcd. For C₃₃H₅₀N₂On₁: 650.34, found: 651.25 [M+H]⁺.

### Step 2: Synthesis of benzyl N-[20-(4-aminophenoxy)-3,6,9,12,15,18-hexaoxaicosan-1-yl]carbamate

The procedure was the same as methyl 4-[4-(4-amino-1-methylpyrrole-2-amido)-1-methylimidazole-2-amido]-1-methylpyrrole-2-carboxylate (Example 2 Step 4). 1.30 g of benzyl N-(20-{4-[(tert-butoxycarbonyl)amino]phenoxy}-3,6,9,12,15,18-hexaoxaicosan-1-yl)carbamate was used, 1.30 g crude of benzyl N-[20-(4-aminophenoxy)-3,6,9,12,15,18-hexaoxaicosan-1-yl]carbamate was obtained as yellow oil. LC/MS: mass calcd. For C₂₈H₄₂N₂O₉: 550.29, found: 551.40 [M+H]⁺.

### Step 3: Synthesis of benzyl N-[20-(4-{4-[2-(4-fluoro-2,6-dimethylphenoxy)-5-(2-hydroxypropan-2-yl)phenyl]-6-methyl-7-oxo-1H-pyrrolo[2,3-c]pyridine-2-amido}phenoxy)-3,6,9,12,15,18-hexaoxaicosan-1-yl]carbamate

The procedure was the same as methyl 1-methyl-4-(1-methyl-4-{1-methyl-4-[1-methyl-4-(1-methylimidazole-2-amido)pyrrole-2-amido]pyrrole-2-amido}imidazole-2-amido)pyrrole-2-carboxylate. 900.00 mg of 4-[2-(4-fluoro-2,6-dimethylphenoxy)-5-(2-hydroxypropan-2-yl)phenyl]-6-methyl-7-oxo-1H-pyrrolo[2,3-c]pyridine-2-carboxylic acid was used, 1.40 g of benzyl N-[20-(4-{4-[2-(4-fluoro-2,6-dimethylphenoxy)-5-(2-hydroxypropan-2-yl)phenyl]-6-methyl-7-oxo-1H-pyrrolo[2,3-c]pyridine-2-amido}phenoxy)-3,6,9,12,15,18-hexaoxaicosan-1-yl]carbamate was obtained as white solid (72.46% yield).

LC/MS: mass calcd. For C₅₄H₆₅FN₄O₁₃: 996.45, found: 1019.30 [M+Na]⁺.

### Step 4: Synthesis of N-{4-[(20-amino-3,6,9,12,15,18-hexaoxaicosan-1-yl)oxy]phenyl}-4-[2-(4-fluoro-2,6-dimethylphenoxy)-5-(2-hydroxypropan-2-yl)phenyl]-6-methyl-7-oxo-1H-pyrrolo[2,3-c]pyridine-2-carboxamide

To a solution of benzyl N-[20-(4-{4-[2-(4-fluoro-2,6-dimethylphenoxy)-5-(2-hydroxypropan-2-yl)phenyl]-6-methyl-7-oxo-1H-pyrrolo[2,3-c]pyridine-2-amido}phenoxy)-3,6,9,12,15,18-hexaoxaicosan-1-yl]carbamate (1400.00 mg, 1.40 mmol, 1.00 equiv) in DMF (50.00 mL) was added Pd/C (400.00 mg, 28% w/w). Then the reaction was stirred for 17.0 h at room temperature under H₂ atmosphere. The mixture was filtrated and the filtrate was concentrated. Then the reaction mixture was purified by reverse flash chromatography with the following conditions: column, C18 silica gel; mobile phase, ACN in water (0.05% NH₄HCO₃), 40% to 50% gradient in 20.0 min; detector, UV 254 n. The fractions were combined and concentrated. N-{4-[(20-amino-3,6,9,12,15,18-hexaoxaicosan-1-yl)oxy]phenyl}-4-[2-(4-fluoro-2,6-dimethylphenoxy)-5-(2-hydroxypropan-2-yl)phenyl]-6-methyl-7-oxo-1H-pyrrolo[2,3-c]pyridine-2-carboxamide (1000.00 mg, 82.53%) was obtained as yellow oil. LC/MS: mass calcd. For C₄₆H₅₉FN₄O₁₁: 862.42, found: 863.30 [M+H]⁺.

### Step 5: Synthesis of Compound 251

The procedure was the same as N-(5-{[2-({2-[(2-{[26-(4-{1-ethyl-4-[2-(4-fluoro-2,6-dimethylphenoxy)-5-(2-hydroxypropan-2-yl)phenyl]-6-methyl-7-oxopyrrolo[2,3-c]pyridine-2-amido}phenoxy)-3,6,9,12,15,18,21,24-octaoxahexacosan-1-yl]carbamoyl}ethyl)carbamoyl]-1-methylimidazol-4-yl}carbamoyl)ethyl]carbamoyl}-1-methylpyrrol-3-yl)-1-methyl-4-(3-{[1-methyl-4-(1-methylimidazole-2-amido)pyrrol-2-yl]formamido}propanamido)imidazole-2-carboxamide (Example 20). 60.00 mg of 3-({1-methyl-4-[3-({4-[1-methyl-4-(3-{[1-methyl-4-(1-methylimidazole-2-amido)pyrrol-2-yl]formamido}propanamido)imidazole-2-amido]-1-[14-(2,5,8,11-tetraoxatridecan-13-yl)-2,5,8,11-tetraoxa-14-azanonadecan-19-yl]pyrrol-2-yl}formamido)propanamido]imidazol-2-yl}formamido)propanoic acid was used, 1.20 mg of N-(5-{[2-({2-[(2-{[20-(4-{4-[2-(4-fluoro-2,6-dimethylphenoxy)-5-(2-hydroxypropan-2-yl)phenyl]-6-methyl-7-oxo-1H-pyrrolo[2,3-c]pyridine-2-amido}phenoxy)-3,6,9,12,15,18-hexaoxaicosan-1-yl]carbamoyl}ethyl)carbamoyl]-1-methylimidazol-4-yl}carbamoyl)ethyl]carbamoyl}-1-[14-(2,5,8,11-tetraoxatridecan-13-yl)-2,5,8,11-tetraoxa-14-azanonadecan-19-yl]pyrrol-3-yl)-1-methyl-4-(3-{[1-methyl-4-(1-methylimidazole-2-amido)pyrrol-2-yl]formamido}propanamido)imidazole-2-carboxamide was obtained as white solid (1.18% yield). HRMS: mass calcd. For C₁₀₄H₁₄₅FN₂00n: 2125.0572, found: 2126.0606 [M+H]⁺.

### Example 47. Synthesis of N-(5-{[2-({2-[(2-{[26-(4-{2-[(9S)-7-(4-chlorophenyl)-4,5,13-trimethyl-3-thia-1,8,11,12-tetraazatricyclo[8.3.0.0{2,6}]trideca-2(6), 4,7,10,12-pentaen-9-yl]acetamido}-2-(2,5,8,11,14,17-hexaoxanonadecan-19-yloxy)phenoxy)-3,6,9,12,15,18,21,24-octaoxahexacosan-1-yl]carbamoyl}ethyl)carbamoyl]-1-methylimidazol-4-yl]carbamoyl)ethyl]carbamoyl]-1-methylpyrrol-3-yl)-1-methyl-4-(3-{[1-methyl-4-(1-methylimidazole-2-amido)pyrrol-2-yl]formamido]propanamido)imidazole-2-carboxamide (Compound 211)

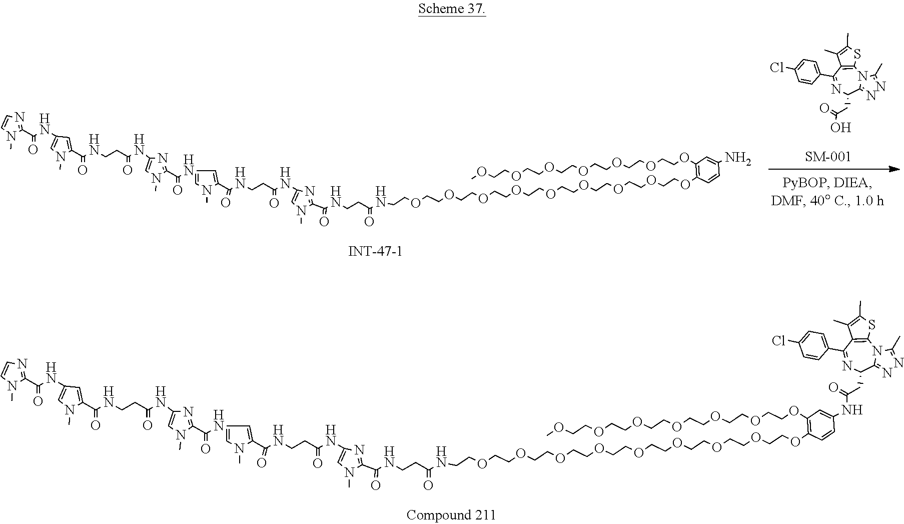 [SMILES: C=CN(C)/C(=C\NC(=O)c1nc(NC(=O)CCNC(=O)c2cc(NC(=O)c3nccn3C)cn2C)cn1C)C(=O)NCCC(=O)Nc1cn(C)c(C(=O)NCCC(=O)NCCOCCOCCOCCOCCOCCOCCOCCOCCOc2ccc(N)cc2OCCOCCOCCOCCOCCOCCOC)n1 ; Cc1sc2c(c1C)C(c1ccc(Cl)cc1)=N[C@@H](CC(=O)O)c1nnc(C)n1-2 ; C=CN(C)/C(=C\NC(=O)c1nc(NC(=O)CCNC(=O)c2cc(NC(=O)c3nccn3C)cn2C)cn1C)C(=O)NCCC(=O)Nc1cn(C)c(C(=O)NCCC(=O)NCCOCCOCCOCCOCCOCCOCCOCCOCCOc2ccc(NC(=O)C[C@@H]3N=C(c4ccc(Cl)cc4)c4c(sc(C)c4C)-n4c(C)nnc43)cc2OCCOCCOCCOCCOCCOCCOC)n1]

The procedure was the same as N-(5-{[2-({2-[(2-{[26-(4-{1-ethyl-4-[2-(4-fluoro-2,6-dimethylphenoxy)-5-(2-hydroxypropan-2-yl)phenyl]-6-methyl-7-oxopyrrolo[2,3-c]pyridine-2-amido}phenoxy)-3,6,9,12,15,18,21,24-octaoxahexacosan-1-yl]carbamoyl}ethyl)carbamoyl]-1-methylimidazol-4-yl}carbamoyl)ethyl]carbamoyl}-1-methylpyrrol-3-yl)-1-methyl-4-(3-{[1-methyl-4-(1-methylimidazole-2-amido)pyrrol-2-yl]formamido}propanamido)imidazole-2-carboxamide (Example 20). 200.00 mg of N-[5-({2-[(2-{[2-({26-[4-amino-2-(2,5,8,11,14,17-hexaoxanonadecan-19-yloxy)phenoxy]-3,6,9,12,15,18,21,24-octaoxahexacosan-1-yl}carbamoyl)ethyl]carbamoyl}-1-methylimidazol-4-yl)carbamoyl]ethyl}carbamoyl)-1-methylpyrrol-3-yl]-1-methyl-4-(3-{[1-methyl-4-(1-methylimidazole-2-amido)pyrrol-2-yl]formamido}propanamido)imidazole-2-carboxamide was used, 40.20 mg of N-(5-{[2-({2-[(2-{[26-(4-{2-[(9S)-7-(4-chlorophenyl)-4,5,13-trimethyl-3-thia-1,8,11,12-tetraazatricyclo[8.3.0.0{2,6}]trideca-2(6), 4,7,10,12-pentaen-9-yl]acetamido}-2-(2,5,8,11,14,17-hexaoxanonadecan-19-yloxy)phenoxy)-3,6,9,12,15,18,21,24-octaoxahexacosan-1-yl]carbamoyl}ethyl)carbamoyl]-1-methylimidazol-4-yl}carbamoyl)ethyl]carbamoyl}-1-methylpyrrol-3-yl)-1-methyl-4-(3-{[1-methyl-4-(1-methylimidazole-2-amido)pyrrol-2-yl]formamido}propanamido)imidazole-2-carboxamide was obtained as white solid (15.92% yield). HRMS: mass calcd. For C₉₂H₁₂₆ClN₂₁O₂₅S: 1991.8642, found: 1992.8556 [M+H]⁺.

### Example 48. Synthesis of N-(5-{[2-({2-[(2-{[26-(4-{2-[(9S)-7-(4-chlorophenyl)-4,5,13-trimethyl-3-thia-1,8,11,12-tetraazatricyclo[8.3.0.0{2,6}]trideca-2(6), 4,7,10,12-pentaen-9-yl]acetamido}phenoxy)-3,6,9,12,15,18,21,24-octaoxahexacosan-1-yl]carbamoyl]ethyl)carbamoyl]-1-methylimidazol-4-yl]carbamoyl)ethyl]carbamoyl]-1H-pyrrol-3-yl)-1-methyl-4-(3-{[4-(1-methylimidazole-2-amido)-1H-pyrrol-2-yl]formamido]propanamido)imidazole-2-carboxamide (Compound 239)

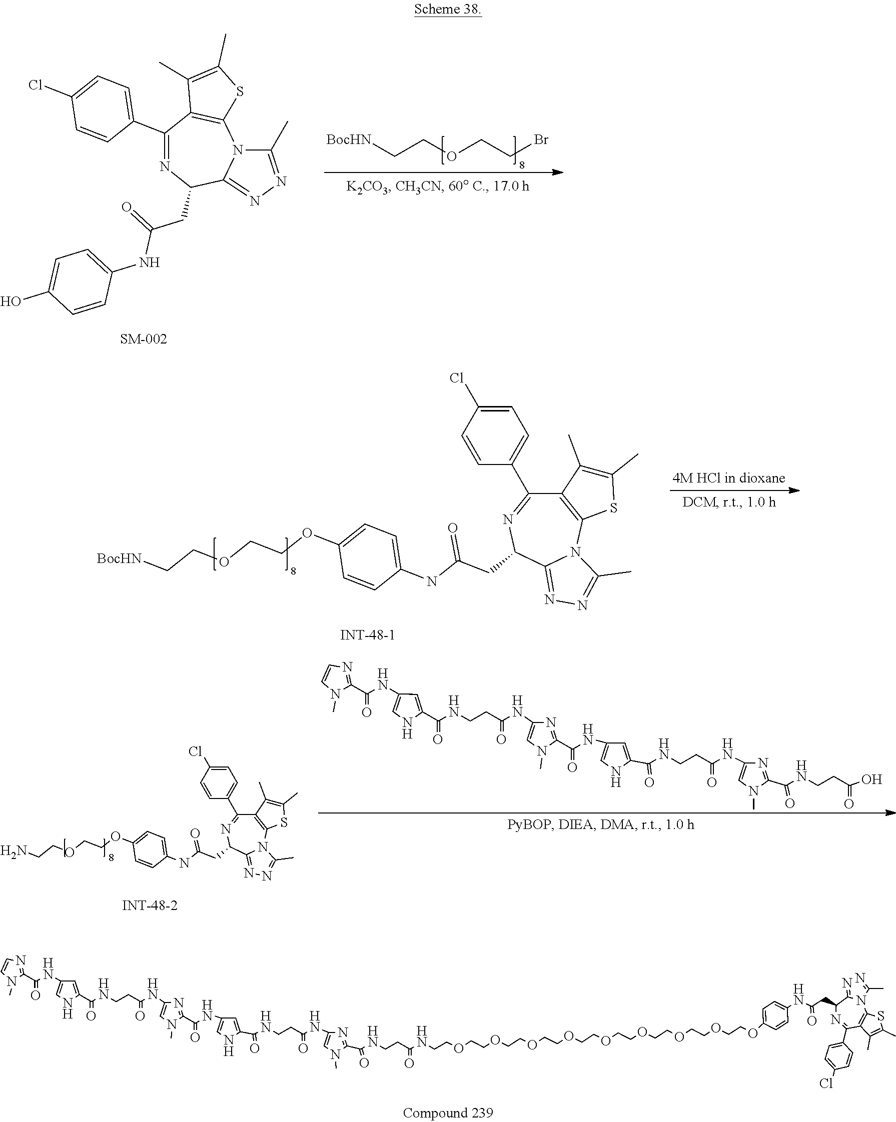

### Step 1: Synthesis of tert-butyl (S)-(26-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15,18,21,24-octaoxahexacosyl)carbamate

The procedure was the same as ethyl 1-(5-bromopentyl)-4-[(tert-butoxycarbonyl)amino]pyrrole-2-carboxylate (Example 9 Step 4), but the reaction temperature was 60 degrees C. and the reaction time was 17.0 h. 150.00 mg of (S)-2-(4-(4-chlorophenyl)-2,3,9-trimethyl-6H-thieno[3,2-f][1,2,4]triazolo[4,3-a][1,4]diazepin-6-yl)-N-(4-hydroxyphenyl)acetamide was used, 350.00 mg of tert-butyl (S)-(26-(4-(2-(4-(4-chlorophenyl)-2,3,9-trimethyl-6H-thieno[3,2-f][1,2,4]triazolo[4,3-a][1,4]diazepin-6-yl)acetamido)phenoxy)-3,6,9,12,15,18,21,24-octaoxahexacosyl)carbamate was obtained as white solid (87% purity, 100% yield).

LC/MS: mass calcd. For C₄₈H₆₇ClN₆O₁₂S: 986.42, found: 1009.60 [M+Na]⁺.

### Step 2: Synthesis of N-{4-[(26-amino-3,6,9,12,15,18,21,24-octaoxahexacosan-1-yl)oxy]phenyl}-2-[(9S)-7-(4-chlorophenyl)-4,5,13-trimethyl-3-thia-1,8,11,12-tetraazatricyclo[8.3.0.0{2,6}]trideca-2(6), 4,7,10,12-pentaen-9-yl]acetamide (INT-055-102)

The procedure was the same as methyl 4-[4-(3-aminopropanamido)-1-methylimidazole-2-amido]-1-methylpyrrole-2-carboxylate hydrochloride (Example 4 Step 1), but the reaction solvent was 4M HCl in dioxane/DCM (1:1) and the reaction time was 1.0 h. 80.00 mg of tert-butyl N-[26-(4-{2-[(9S)-7-(4-chlorophenyl)-4,5,13-trimethyl-3-thia-1,8,11,12-tetraazatricyclo[8.3.0.0{2,6}]trideca-2(6), 4,7,10,12-pentaen-9-yl]acetamido}phenoxy)-3,6,9,12,15,18,21,24-octaoxahexacosan-1-yl]carbamate was used, 80.00 mg crude of N-{4-[(26-amino-3,6,9,12,15,18,21,24-octaoxahexacosan-1-yl)oxy]phenyl}-2-[(9S)-7-(4-chlorophenyl)-4,5,13-trimethyl-3-thia-1,8,11,12-tetraazatricyclo[8.3.0.0{2,6}]trideca-2(6), 4,7,10,12-pentaen-9-yl]acetamide was obtained as yellow oil. LC/MS: mass calcd. For C₄₃H₅₉ClN₆O₁₀S: 886.37, found: 444.45 [M/2+H]⁺.

### Step 3: Synthesis of Compound 239

The procedure was the same as N-(5-{[2-({2-[(2-{[26-(4-{1-ethyl-4-[2-(4-fluoro-2,6-dimethylphenoxy)-5-(2-hydroxypropan-2-yl)phenyl]-6-methyl-7-oxopyrrolo[2,3-c]pyridine-2-amido}phenoxy)-3,6,9,12,15,18,21,24-octaoxahexacosan-1-yl]carbamoyl}ethyl)carbamoyl]-1-methylimidazol-4-yl}carbamoyl)ethyl]carbamoyl}-1-methylpyrrol-3-yl)-1-methyl-4-(3-{[1-methyl-4-(1-methylimidazole-2-amido)pyrrol-2-yl]formamido}propanamido)imidazole-2-carboxamide (Example 20), but the reaction solvent was DMA. 70.00 mg of N-{4-[(26-amino-3,6,9,12,15,18,21,24-octaoxahexacosan-1-yl)oxy]phenyl}-2-[(9S)-7-(4-chlorophenyl)-4,5,13-trimethyl-3-thia-1,8,11,12-tetraazatricyclo[8.3.0.0{2,6}]trideca-2(6), 4,7,10,12-pentaen-9-yl]acetamide was used, 20.80 mg of N-(5-{[2-({2-[(2-{[26-(4-{2-[(9S)-7-(4-chlorophenyl)-4,5,13-trimethyl-3-thia-1,8,11,12-tetraazatricyclo[8.3.0.0\hat{ }{2,6}]trideca-2(6), 4,7,10,12-pentaen-9-yl]acetamido}phenoxy)-3,6,9,12,15,18,21,24-octaoxahexacosan-1-yl]carbamoyl}ethyl)carbamoyl]-1-methylimidazol-4-yl}carbamoyl)ethyl]carbamoyl}-1H-pyrrol-3-yl)-1-methyl-4-(3-{[4-(1-methylimidazole-2-amido)-1H-pyrrol-2-yl]formamido}propanamido)imidazole-2-carboxamide was obtained as white solid (15.53% yield).

HRMS: mass calcd. For C₇₇H₉₆ClN₂O₁₈S Exact Mass: 1669.6651, found: 1670.6690 [M+H]⁺.

### Example 49. Synthesis of (S)—N-(5-((3-((2-((22-(5-(4-(2-(4-(4-chlorophenyl)-2,3,9-trimethyl-6H-thieno[3,2-f][1,2,4]triazolo[4,3-a][1,4diazepin-6-yl)acetamido)phenyl)penta-2,4-diyn-1-yl)-3,23-dioxo-7,10,13,16,19-pentaoxa-4,22-diazaoctatriacontyl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide (Compound 202)

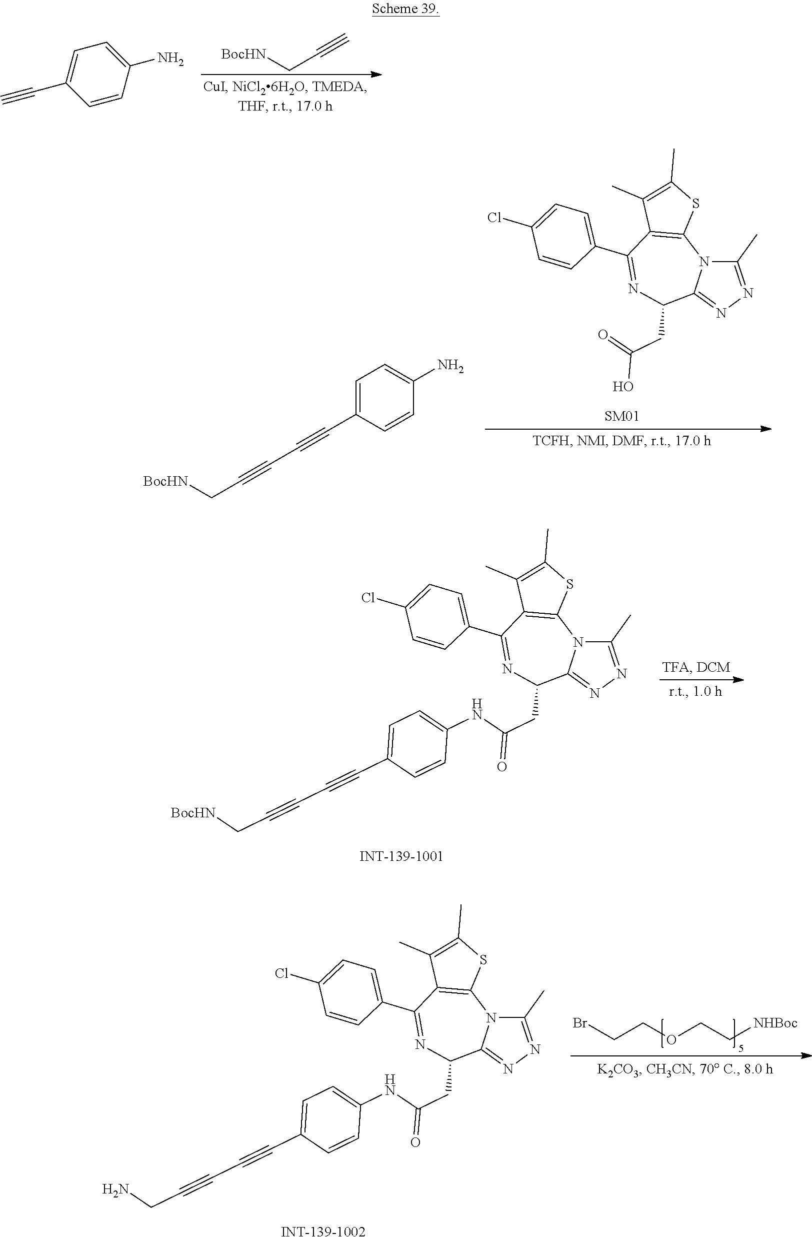

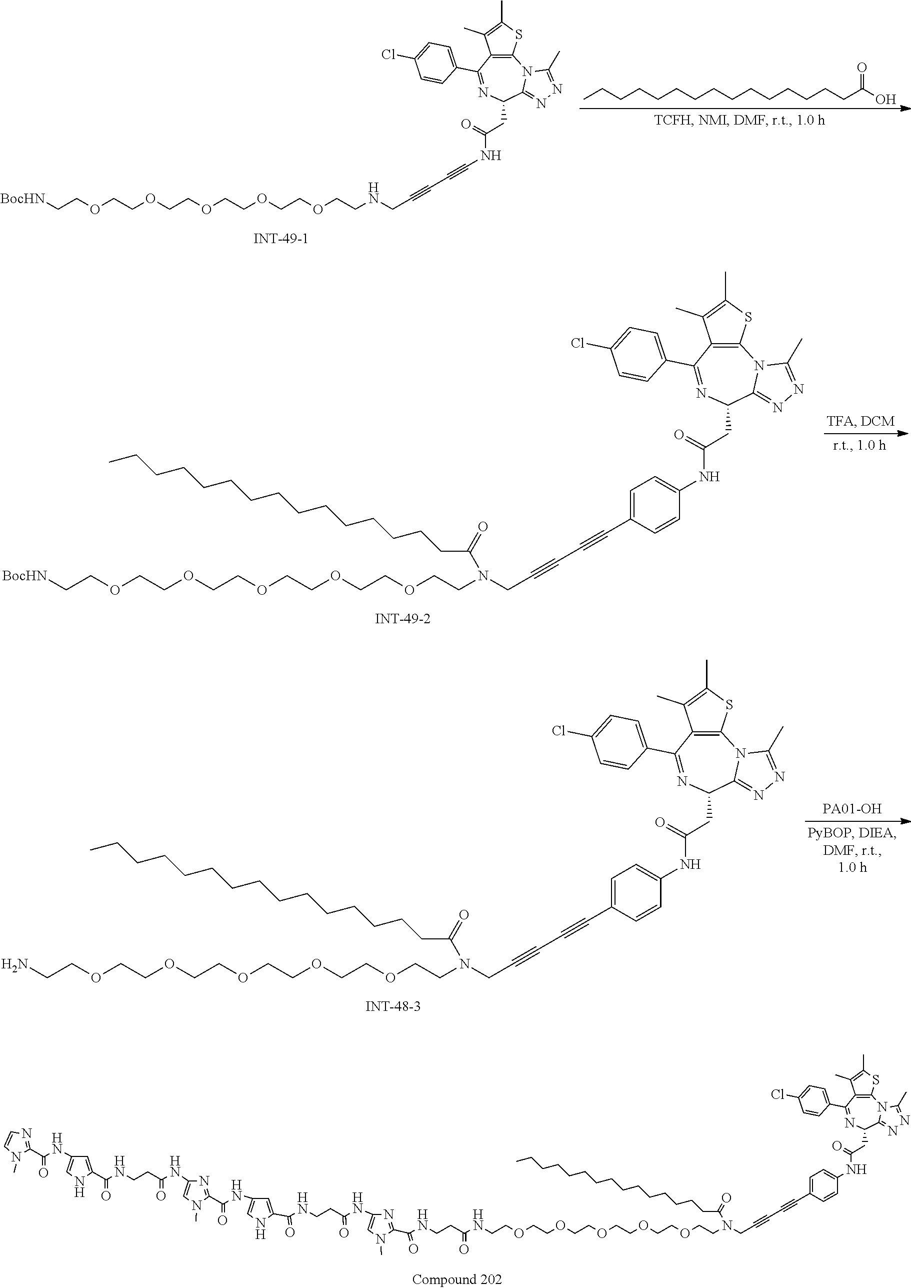

### Step 1: Synthesis of tert-butyl N-[5-(4-aminophenyl)penta-2,4-diyn-1-yl]carbamate

To a stirred solution of CuI (56.90 mg, 0.299 mmol, 0.05 equiv) and NiCl₂·6H₂O(71.01 mg, 0.299 mmol, 0.05 equiv) in THF (20.00 mL) was added TMEDA (138.87 mg, 1.195 mmol, 0.20 equiv) dropwise at room temperature under air atmosphere. The resulting mixture was stirred for 5.0 min at room temperature under air atmosphere. To the above mixture was added 4-ethynylaniline (700.00 mg, 5.975 mmol, 1.00 equiv) and tert-butyl N-(prop-2-yn-1-yl)carbamate (463.67 mg, 2.988 mmol, 0.50 equiv) in THF (10.00 mL) in portions at room temperature. The resulting mixture was stirred for additional 17.0 h at room temperature. The reaction was quenched by the addition of H₂O (50 mL) at room temperature. The resulting mixture was extracted with EA (3×80 mL). The combined organic layers were washed with brine (1×50 mL), dried over anhydrous Na2SO4. After filtration, the filtrate was concentrated under reduced pressure. The residue was purified by s ilica gel column chromatography, eluted with PE/EA (3:1) to afford tert-butyl N-[5-(4-aminophenyl)penta-2,4-diyn-1-yl]carbamate (600.00 mg, 37.15%) as yellow solid. LC/MS: mass calcd. For C₁₆H₁₈N₂O₂: 270.14, found: 541.30, 215.05 [2M+H, M-tBu+H]⁺.

### Step 2: Synthesis of tert-butyl (S)-(5-(4-(2-(4-(4-chlorophenyl)-2,3,9-trimethyl-6H-thieno[3,2-f][1,2,4]triazolo[4,3-a][1,4]diazepin-6-yl)acetamido)phenyl)penta-2,4-diyn-1-yl)carbamate

The procedure was the same as ethyl 4-(1-methylimidazole-2-amido)-1-[26-(2,5,8,11,14,17,20,23-octaoxapentacosan-25-yl)-2,5,8,11,14,17,20,23-octaoxa-26-azahentriacontan-31-yl]pyrrole-2-carboxylate, but the reaction time was 17.0 h. 360.00 mg of (S)-2-(4-(4-chlorophenyl)-2,3,9-trimethyl-6H-thieno[3,2-f][1,2,4]triazolo[4,3-a][1,4]diazepin-6-yl)acetic acid was used, 450.00 mg of tert-butyl (S)-(5-(4-(2-(4-(4-chlorophenyl)-2,3,9-trimethyl-6H-thieno[3,2-f][1,2,4]triazolo[4,3-a][1,4]diazepin-6-yl)acetamido)phenyl)penta-2,4-diyn-1-yl)carbamate was obtained as yellow solid (65.60% yield). LC/MS: mass calcd. For C₇₇H₉₆ClN₂₁O₁₈S: C₃₅H₃₃ClN₆O₃S: 652.20, found: 653.20 [M+H]⁺.

### Step 3: Synthesis of (S)—N-(4-(5-aminopenta-1,3-diyn-1-yl)phenyl)-2-(4-(4-chlorophenyl)-2,3,9-trimethyl-6H-thieno[3,2-f][1,2,4]triazolo[4,3-a][1,4]diazepin-6-yl)acetamide

The procedure was the same as methyl 4-[4-(4-amino-1-methylpyrrole-2-amido)-1-methylimidazole-2-amido]-1-methylpyrrole-2-carboxylate (Example 2 Step 3). 450.00 mg of tert-butyl (S)-(5-(4-(2-(4-(4-chlorophenyl)-2,3,9-trimethyl-6H-thieno[3,2-f][1,2,4]triazolo[4,3-a][1,4]diazepin-6-yl)acetamido)phenyl)penta-2,4-diyn-1-yl)carbamate was used, 450.00 mg crude of desired product was obtained as yellow oil. LC/MS: mass calcd. For C₃₀H₂₅ClN₆OS: 552.15, found: 553.20 [M+H]⁺.

### Step 4: Synthesis of tert-butyl (S)-(23-(4-(2-(4-(4-chlorophenyl)-2,3,9-trimethyl-6H-thieno[3,2-f][1,2,4]triazolo[4,3-a][1,4]diazepin-6-yl)acetamido)phenyl)-3,6,9,12,15-pentaoxa-18-azatricosa-20,22-diyn-1-yl)carbamate

The procedure was the same as tert-butyl (S)-2-(4-(4-(16-((2-(1H-indol-3-yl)ethyl)amino)hexadecanamido)phenyl)-2,3,9-trimethyl-6H-thieno[3,2-f][1,2,4]triazolo[4,3-a][1,4]diazepin-6-yl)acetate, but KI (1.50 equiv) was added and the reaction time was 8.0 h. 500.00 mg of (S)—N-(4-(5-aminopenta-1,3-diyn-1-yl)phenyl)-2-(4-(4-chlorophenyl)-2,3,9-trimethyl-6H-thieno[3,2-f][1,2,4]triazolo[4,3-a][1,4]diazepin-6-yl)acetamide was used, 150.00 mg of desired product was obtained as light yellow oil (18.10% yield). LC/MS: mass calcd. For C₄₇H₅₈ClN₇O₈S: 915.38, found: 938.30 [M+Na]⁺.

### Step 5: Synthesis of tert-butyl (S)-(18-(5-(4-(2-(4-(4-chlorophenyl)-2,3,9-trimethyl-6H-thieno[3,2-f][1,2,4]triazolo[4,3-a][1,4]diazepin-6-yl)acetamido)phenyl)penta-2,4-diyn-1-yl)-19-oxo-3,6,9,12,15-pentaoxa-18-azatetratriacontyl)carbamate

The procedure was the same as ethyl 4-(1-methylimidazole-2-amido)-1-[26-(2,5,8,11,14,17,20,23-octaoxapentacosan-25-yl)-2,5,8,11,14,17,20,23-octaoxa-26-azahentriacontan-31-yl]pyrrole-2-carboxylate. 60.00 mg of t-butyl (S)-(23-(4-(2-(4-(4-chlorophenyl)-2,3,9-trimethyl-6H-thieno[3,2-f][1,2,4]triazolo[4,3-a][1,4]diazepin-6-yl)acetamido)phenyl)-3,6,9,12,15-pentaoxa-18-azatricosa-20,22-diyn-1-yl)carbamate was used, 60.00 mg of desired product was obtained as light yellow solid (79.36% yield). LC/MS: mass calcd. For C₆₃H₈₈ClN₇O₉S: 1153.61, found: 1177.00 [M+Na]⁺.

### Step 6: Synthesis of (S)—N-(17-amino-3,6,9,12,15-pentaoxaheptadecyl)-N-(5-(4-(2-(4-(4-chlorophenyl)-2,3,9-trimethyl-6H-thieno[3,2-f][1,2,4]triazolo[4,3-a[1,4]diazepin-6-yl)acetamido)phenyl)penta-2,4-diyn-1-yl)palmitamide

The procedure was the same as methyl 4-[4-(4-amino-1-methylpyrrole-2-amido)-1-methylimidazole-2-amido]-1-methylpyrrole-2-carboxylate (Example 2 Step 4). 60.00 mg of tert-butyl (S)-(18-(5-(4-(2-(4-(4-chlorophenyl)-2,3,9-trimethyl-6H-thieno[3,2-f][1,2,4]triazolo[4,3-a][1,4]diazepin-6-yl)acetamido)phenyl)penta-2,4-diyn-1-yl)-19-oxo-3,6,9,12,15-pentaoxa-18-azatetratriacontyl)carbamate was used, 60.00 mg crude of desired product was obtained as yellow oil. LC/MS: mass calcd. For C₅₈H₈₀ ClN₇O₇S: 1053.55, found: 1054.55 [M+H]⁺.

### Step 7: Synthesis of Compound 202

The procedure was the same as N-(5-{[2-({2-[(2-{[26-(4-{1-ethyl-4-[2-(4-fluoro-2,6-dimethylphenoxy)-5-(2-hydroxypropan-2-yl)phenyl]-6-methyl-7-oxopyrrolo[2,3-c]pyridine-2-amido}phenoxy)-3,6,9,12,15,18,21,24-octaoxahexacosan-1-yl]carbamoyl}ethyl)carbamoyl]-1-methylimidazol-4-yl}carbamoyl)ethyl]carbamoyl}-1-methylpyrrol-3-yl)-1-methyl-4-(3-{[1-methyl-4-(1-methylimidazole-2-amido)pyrrol-2-yl]formamido}propanamido)imidazole-2-carboxamide (Example 20). 55.00 mg of (S)—N-(17-amino-3,6,9,12,15-pentaoxaheptadecyl)-N-(5-(4-(2-(4-(4-chlorophenyl)-2,3,9-trimethyl-6H-thieno[3,2-f][1,2,4]triazolo[4,3-a][1,4]diazepin-6-yl)acetamido)phenyl)penta-2,4-diyn-1-yl)palmitamide was used, 24.70 mg of desired product was obtained as white solid (24.52% yield). HRMS: mass calcd. For C₉₄H₁₂₁ClN₂₂O₁₅S: 1864.8791, found: 1865.8915 [M+H]⁺.

### Example 50. Synthesis of (S)—N-(5-((3-((2-((22-(5-(4-(2-(4-(4-chlorophenyl)-2,3,9-trimethyl-6H-thieno[3,2-f][1,2,4]triazolo[4,3-a][1,4diazepin-6-yl)acetamido)phenyl)penta-2,4-diyn-1-yl)-3,23-dioxo-7,10,13,16,19-pentaoxa-4,22-diazatriacontyl)carbamoyl)-1-methyl-1H-imidazol-4-yl)amino)-3-oxopropyl)carbamoyl)-1-methyl-1H-pyrrol-3-yl)-1-methyl-4-(3-(1-methyl-4-(1-methyl-1H-imidazole-2-carboxamido)-1H-pyrrole-2-carboxamido)propanamido)-1H-imidazole-2-carboxamide (Compound 201)

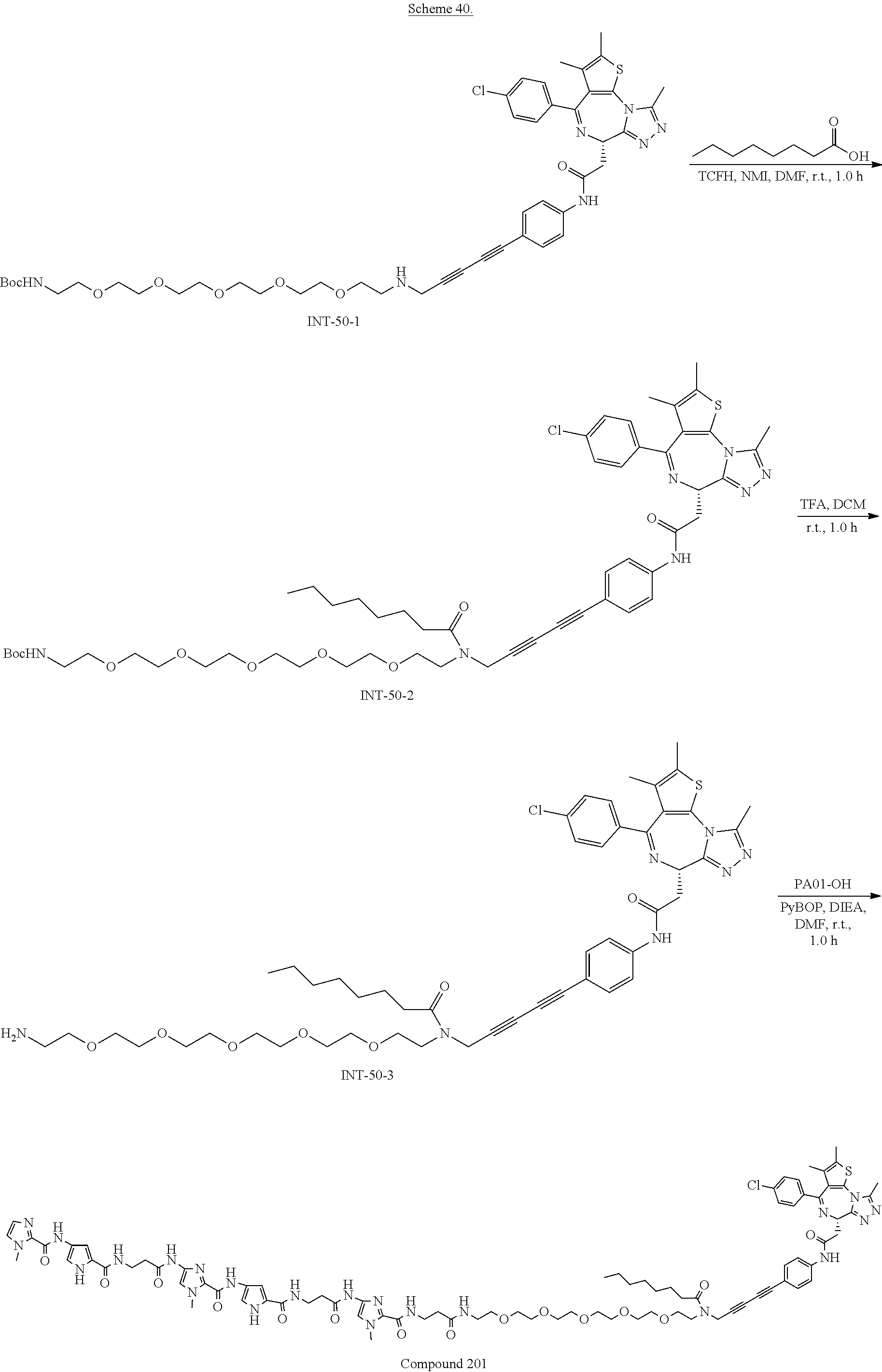

### Step 1: Synthesis of tert-butyl (S)-(18-(5-(4-(2-(4-(4-chlorophenyl)-2,3,9-trimethyl-6H-thieno[3,2-f][1,2,4]triazolo[4,3-a][1,4]diazepin-6-yl)acetamido)phenyl)penta-2,4-diyn-1-yl)-19-oxo-3,6,9,12,15-pentaoxa-18-azahexacosyl)carbamate

The procedure was the same as ethyl 4-(1-methylimidazole-2-amido)-1-[26-(2,5,8,11,14,17,20,23-octaoxapentacosan-25-yl)-2,5,8,11,14,17,20,23-octaoxa-26-azahentriacontan-31-yl]pyrrole-2-carboxylate. 70.00 mg of tert-butyl (S)-(23-(4-(2-(4-(4-chlorophenyl)-2,3,9-trimethyl-6H-thieno[3,2-f][1,2,4]triazolo[4,3-a][1,4]diazepin-6-yl)acetamido)phenyl)-3,6,9,12,15-pentaoxa-18-azatricosa-20,22-diyn-1-yl)carbamate was used, 80.00 mg of desired product was obtained as light yellow solid (94.43% yield). LC/MS: mass calcd. For C₅₅H₇₂ClN₇O₉S: 1041.48, found: 1064.75 [M+Na]⁺.

### Step 2: Synthesis of (S)—N-(17-amino-3,6,9,12,15-pentaoxaheptadecyl)-N-(5-(4-(2-(4-(4-chlorophenyl)-2,3,9-trimethyl-6H-thieno[3,2-f][1,2,4]triazolo[4,3-a][1,4]diazepin-6-yl)acetamido)phenyl)penta-2,4-diyn-1-yl)octanamide

The procedure was the same as methyl 4-[4-(4-amino-1-methylpyrrole-2-amido)-1-methylimidazole-2-amido]-1-methylpyrrole-2-carboxylate (Example 2 Step 4). 70.00 mg of tert-butyl (S)-(18-(5-(4-(2-(4-(4-chlorophenyl)-2,3,9-trimethyl-6H-thieno[3,2-f][1,2,4]triazolo[4,3-a][1,4]diazepin-6-yl)acetamido)phenyl)penta-2,4-diyn-1-yl)-19-oxo-3,6,9,12,15-pentaoxa-18-azahexacosyl)carbamate was used, 70.00 mg crude of desired product was obtained as yellow oil. LC/MS: mass calcd. For C₅₀H₆₄ClN₇O₇S: 941.43, found: 942.40 [M+H]⁺.

### Step 3: Synthesis of Compound 201

The procedure was the same as N-(5-{[2-({2-[(2-{[26-(4-{1-ethyl-4-[2-(4-fluoro-2,6-dimethylphenoxy)-5-(2-hydroxypropan-2-yl)phenyl]-6-methyl-7-oxopyrrolo[2,3-c]pyridine-2-amido}phenoxy)-3,6,9,12,15,18,21,24-octaoxahexacosan-1-yl]carbamoyl}ethyl)carbamoyl]-1-methylimidazol-4-yl}carbamoyl)ethyl]carbamoyl}-1-methylpyrrol-3-yl)-1-methyl-4-(3-{[1-methyl-4-(1-methylimidazole-2-amido)pyrrol-2-yl]formamido}propanamido)imidazole-2-carboxamide (Example 20). 50.20 mg of 3-({1-methyl-4-[3-({1-methyl-4-[1-methyl-4-(3-{[1-methyl-4-(1-methylimidazole-2-amido)pyrrol-2-yl]formamido}propanamido)imidazole-2-amido]pyrrol-2-yl}formamido)propanamido]imidazol-2-yl}formamido)propanoic acid was used, 30.60 mg of desired product was obtained as white solid (25.40% yield). HRMS: mass calcd. For C₈₆H₁₀₅ClN₂₂O₁₅S: 1752.7539, found: 1753.7599 [M+H]⁺.

### Example 51. General synthesis and purification of the compounds of the disclosure

Compounds of the disclosure were made by methods similar to Examples 1-50. The compounds were subsequently purified by HRMS methods A or B.

Method A: Instrument: Waters Acquity I Class UPLC with Xevo G2-XSQ Tof HRMS; Column: ACQUITY UPLC BEH-C18, 2.1×50 mm, 2.7 μm; mobile phase A: H₂O (0.1% HCOOH), mobile B, ACN (0.1% HCOOH); Flow rate: 0.4 mL/min; Gradient: 10% B to 95% B in 1.5 min, hold 95% for another 0.5 min, then down to 10% B in 0.3 min, hold 10% B for another 0.7 min; detector: 254 n.

Method B: Instrument: Waters AcquityI Class UPLC with Xevo G2-XS Q Tof HRMS; Column: ACQUITY UPLC BEH-C18, 2.1×50 mm, 2.7 μm; mobile phase A: H₂O (0.1% HCOOH), mobile B, ACN (0.1% HCOOH); Flow rate: 0.4 mL/min; Gradient: 5% B to 40% B in 2.0 min, to 95% in another 1.5 min, hold 95% for 1.5 min, then down to 5% B in 0.3 min, hold 5% B for another 0.7 min; detector: 254 n.

Experimental data for compounds 1-250 purified by Method A are provided in Table 7.

TABLE 7
LCMS analysis of compounds of the disclosure.

| Cmpd. | Observed [M + H]+ from |
|---|---|
| No. | TOF-HRMS [m/z] |
| 1 | 1759.7784 |
| 2 | 1722.757 |
| 3 | 1678.7284 |
| 4 | 1634.6936 |
| 5 | 1731.7936 |
| 6 | 1687.7572 |
| 7 | 1643.7393 |
| 8 | 1761.7981 |
| 9 | 1717.774 |
| 10 | 1673.7518 |
| 11 | 1701.7356 |
| 12 | 1657.7103 |
| 13 | 1613.6779 |
| 14 | 1671.6852 |
| 15 | 1627.6539 |
| 16 | 1583.6356 |
| 17 | 1694.8671 |
| 18 | 1806.8904 |
| 19 | 1762.866 |
| 20 | 1718.8354 |
| 21 | 1741.8245 |
| 22 | 1717.7799 |
| 23 | 1713.7932 |
| 24 | 1709.8192 |
| 25 | 1739.8094 |
| 26 | 1735.8337 |
| 27 | 1699.7806 |
| 28 | 1695.8016 |
| 29 | 1737.8431 |
| 30 | 1733.8743 |
| 31 | 1737.8524 |
| 32 | 1733.8651 |
| 33 | 1722.8333 |
| 34 | 1718.8568 |
| 35 | 1722.8303 |
| 39 | 1713.7972 |
| 40 | 1619.6885 |
| 41 | 1615.7177 |
| 42 | 1767.7472 |
| 43 | 1752.7794 |
| 46 | 1724.8149 |
| 47 | 1753.8478 |
| 48 | 1606.787 |
| 49 | 1762.7982 |
| 50 | 1670.7762 |
| 51 | 1670.7601 |
| 52 | 1666.7797 |
| 53 | 1670.7494 |
| 54 | 1666.7828 |
| 55 | 1728.8003 |
| 56 | 1724.8229 |
| 57 | 1684.7749 |
| 58 | 2374.0754 |
| 60 | 1629.7013 |
| 61 | 1625.7344 |
| 62 | 1642.7274 |
| 63 | 1642.7283 |
| 68 | 1806.8164 |
| 69 | 1806.8131 |
| 70 | 1842.8486 |
| 71 | 1842.8395 |
| 73 | 1924.8484 |
| 74 | 2589.2637 |
| 76 | 2369.1313 |
| 78 | 1663.7203 |
| 79 | 1575.6573 |
| 80 | 1531.6392 |
| 81 | 1784.8369 |
| 82 | 1784.8409 |
| 86 | 1741.8304 |
| 87 | 1717.7896 |
| 88 | 1689.7484 |
| 89 | 1700.7604 |
| 90 | 1728.7975 |
| 91 | 1713.8043 |
| 92 | 1702.7657 |
| 93 | 1701.7599 |
| 98 | 1714.7814 |
| 99 | 1728.7966 |
| 101 | 1675.7399 |
| 104 | 1890.8241 |
| 105 | 2702.1687 |
| 106 | 1713.8044 |
| 107 | 1711.8174 |
| 108 | 1713.8041 |
| 109 | 1711.8168 |
| 111 | 2206.9001 |
| 112 | 2285.0044 |
| 113 | 2026.8436 |
| 114 | 2114.9036 |
| 115 | 1686.8503 |
| 116 | 1672.8309 |
| 117 | 2452.0242 |
| 118 | 2255.9763 |
| 119 | 1610.7531 |
| 120 | 2137.0552 |
| 121 | 1980.0142 |
| 122 | 1767.7653 |
| 123 | 1758.8295 |
| 124 | 1762.7861 |
| 125 | 1762.7943 |
| 126 | 1573.6498 |
| 127 | 1661.694 |
| 130 | 1637.705 |
| 131 | 1784.818 |
| 132 | 1867.8894 |
| 133 | 2156.9954 |
| 134 | 1784.8109 |
| 135 | 1693.8136 |
| 136 | 1546.7422 |
| 138 | 1541.6638 |
| 139 | 1585.6895 |
| 140 | 1673.736 |
| 141 | 1717.7688 |
| 142 | 1761.7905 |
| 143 | 1805.8169 |
| 144 | 1575.7281 |
| 145 | 1663.7704 |
| 150 | 1751.8215 |
| 151 | 1730.7976 |
| 152 | 1704.7772 |
| 154 | 1852.8546 |
| 155 | 1570.6904 |
| 156 | 1614.7101 |
| 157 | 1790.8109 |
| 158 | 1766.869 |
| 159 | 1676.7404 |
| 160 | 1497.6263 |
| 161 | 1453.6089 |
| 162 | 1410.5599 |
| 163 | 1443.5833 |
| 164 | 1356.5173 |
| 165 | 1786.8582 |
| 166 | 1825.9041 |
| 167 | 1748.8741 |
| 168 | 1748.8748 |
| 169 | 1870.9155 |
| 170 | 1870.9175 |
| 171 | 1757.8241 |
| 172 | 1698.7817 |
| 175 | 1854.9087 |
| 176 | 1634.7877 |
| 177 | 1674.7563 |
| 178 | 1586.7029 |
| 179 | 1542.6744 |
| 180 | 1498.6475 |
| 186 | 1522.7229 |
| 195 | 1805.8213 |
| 196 | 1789.8264 |
| 197 | 1744.7899 |
| 198 | 1490/6778 |
| 199 | 1532.7252 |
| 200 | 2076.0676 |
| 201 | 1753.7599 |
| 202 | 1865.8915 |
| 203 | 1805.8650 |
| 204 | 1917.9832 |
| 205 | 1718.7775 |
| 207 | 1772.6914 |
| 211 | 1992.8556 |
| 214 | 1548.7017 |
| 215 | 1542.6900 |
| 217 | 1728.7740 |
| 219 | 17177741 |
| 220 | 1719.7988 |
| 222 | 1703.7563 |
| 223 | 1663.7394 |
| 224 | 1734.7781 |
| 231 | 1778.7906 |
| 240 | 1670.6690 |
| 241 | 2224.9542 |
| 242 | 2214.1118 |
| 244 | 2228.0912 |
| 250 | 2210.0817 |

### BIOLOGICAL EXAMPLES

### Example B1. Biological Activity Assays

Expression of a target gene containing CAG or CTG repeats will be assayed by techniques known in the field. These assays include, but are not limited to quantitative reverse transcription polymerase chain reaction (RT-PCR), microarray, or multiplexed RNA sequencing (RNA-seq, with the chosen assay measuring either total expression, or the allele specific expression of the target gene. Exemplary assays are found at: Freeman W M et al., “Quantitative RT-PCR: pitfalls and potential”, BioTechniques 1999, 26, 112-125; Dudley A M et al, “Measuring absolute expression with microarrays with a calibrated reference sample and an extended signal intensity range”, PNAS USA 2002, 99(11), 7554-7559; Wang Z et al., “RNA-Seq: a revolutionary tool for transcriptomics” Nature Rev. Genetics 2009, 10, 57-63.

Production of the translation product of the target gene will be assayed by techniques known in the field. These assays include, but are not limited to Western blot assay, with the chosen assay measuring either total protein expression, or allele specific expression of the target gene.

For use in assay, two tissue models and two animal models are contemplated.

### Example B2: ECso Assay

Cell culture: Cells were cultured in RPMI1640 medium+15% FBS. Cells were maintained at a density between 2e5/mL and 1e6/mL. Cells were centrifuged, resuspended in fresh medium, counted and plated at 150,000 cells per well in 100 uL in a non-coated, flat bottom tissue culture plate.

Compound treatment: 10 mM stock solution of FA GeneTAC was diluted 1:10 in DMSO followed by a 1:100 dilution in growth medium. Working solution was then further diluted to 10× desired final concentration of 150 nM. Compound was then diluted at a 1:3 ratio into growth medium containing 0.01% DMSO. 5-point, 3-fold dose response curve was generated. 11 μL of 1OX compound was added to wells containing 100 μL cell suspension of GM15850. 11 μL growth medium containing 0.01% DMSO was added to all wells not treated with FA GeneTAC. Cells were allowed to incubate for 48 hrs prior to cell lysis using guanidine isothiocyanate solution.

RNA isolation: Total RNA was isolated and purified in 384-well column filter plates using chaotropic salt.

qRT-PCR: qRT-PCR reactions were assembled using AgPath-ID reagents (Thermo Fisher) using 6 uL mastermix and 4 μL RNA. qRT-PCR TaqMan primer probe sets against human FXN (Assay ID Hs01075496_m1) and human GAPDH (Assay ID Hs00266705_g1) were used to measure the intended targets. qRT-PCR was run on the ThermoFisher QuantStudio 6 PRO instrument using the manufacturer's recommended cycling conditions.

Data analysis: qPCR data was analyzed using Thermo Fisher Design and Analysis software. Data was exported to Excel and hFXN expression was normalized to hGAPDH expression

Representative in vitro biochemical data is presented in Table 8. A<100 nM; B is 100 nM to 500 nM; C>500 nM.

TABLE 8
In vitro potency data.

| Cmpd. | EC₅₀ WT |
|---|---|
| No. | (nM, 48 hr) |
| 2 | A |
| 3 | A |
| 4 | A |
| 5 | B |
| 6 | B |
| 7 | B |
| 8 | A |
| 9 | A |
| 10 | A |
| 11 | A |
| 12 | A |
| 13 | A |
| 14 | B |
| 15 | A |
| 16 | A |
| 18 | C |
| 19 | C |
| 20 | C |
| 21 | C |
| 22 | A |
| 23 | B |
| 24 | B |
| 25 | C |
| 26 | C |
| 27 | C |
| 28 | C |
| 29 | C |
| 33 | C |
| 34 | C |
| 35 | B |
| 39 | C |
| 40 | A |
| 41 | A |
| 42 | A |
| 43 | B |
| 46 | C |
| 47 | A |
| 48 | C |
| 50 | A |
| 51 | B |
| 52 | C |
| 53 | B |
| 54 | C |
| 55 | B |
| 56 | B |
| 57 | B |
| 58 | B |
| 60 | A |
| 61 | A |
| 62 | B |
| 63 | B |
| 68 | A |
| 69 | A |
| 70 | B |
| 71 | B |
| 73 | B |
| 74 | C |
| 76 | B |
| 78 | A |
| 79 | A |
| 80 | A |
| 82 | C |
| 84 | C |
| 86 | B |
| 87 | A |
| 88 | A |
| 89 | A |
| 90 | B |
| 91 | A |
| 92 | A |
| 93 | B |
| 98 | B |
| 99 | B |
| 101 | C |
| 104 | B |
| 106 | A |
| 107 | B |
| 108 | A |
| 109 | B |
| 111 | A |
| 112 | B |
| 113 | A |
| 114 | A |
| 115 | C |
| 116 | C |
| 117 | A |
| 118 | A |
| 119 | A |
| 120 | B |
| 121 | B |
| 122 | B |
| 123 | A |
| 124 | A |
| 125 | A |
| 126 | A |
| 127 | A |
| 130 | A |
| 131 | B |
| 132 | B |
| 133 | B |
| 134 | B |
| 135 | B |
| 136 | C |
| 138 | A |
| 139 | A |
| 140 | A |
| 141 | A |
| 142 | A |
| 143 | A |
| 144 | A |
| 145 | A |
| 150 | A |
| 151 | A |
| 155 | A |
| 156 | A |
| 157 | A |
| 158 | A |
| 159 | A |
| 160 | A |
| 161 | A |
| 163 | A |
| 164 | C |
| 167 | C |
| 168 | B |
| 169 | B |
| 170 | C |
| 171 | A |
| 172 | A |
| 175 | A |
| 176 | A |
| 195 | A |
| 197 | B |
| 199 | C |
| 200 | C |
| 201 | C |
| 203 | B |
| 204 | C |
| 205 | A |
| 207 | A |
| 211 | B |
| 215 | C |
| 217 | A |
| 224 | A |
| 229 | A |
| 231 | A |
| 240 | A |
| 241 | C |
| 242 | A |
| 250 | C |

While preferred embodiments of the present invention have been shown and described herein, it will be obvious to those skilled in the art that such embodiments are provided by way of example only.

Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### ASPECTS

Aspect 1. A transcription modulator molecule having a first terminus, a second terminus, and an oligomeric backbone, wherein:
a) the first terminus comprises a DNA-binding moiety capable of noncovalently binding to a nucleotide repeat sequence GAA,
b) the second terminus comprises a protein-binding moiety binding to a regulatory molecule that modulates an expression of a gene comprising the nucleotide repeat sequence GAA; and
c) the oligomeric backbone comprising a linker between the first terminus and the second terminus.

Aspect 2. The transcription modulator molecule of Aspect 1, wherein the first terminus comprises a linear polyamide.

Aspect 3. The transcription modulator molecule of Aspect 1 or 2, wherein the polyamide is capable of binding the DNA with an affinity of less than 500 nM.

Aspect 4. The transcription modulator molecule of any one of Aspects 1-3, wherein the first terminus comprises a structure of Formula (A-2), or a pharmaceutically acceptable salt thereof:

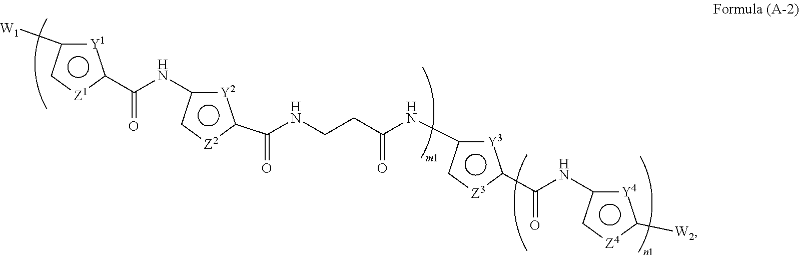

wherein;
m₁ is 1-4;
n₁ is 0-2;
each Y¹, Y², Y³, and Y⁴ is independently CH or N;
each Z¹, Z², Z³, and Z⁴ is independently O, S, or NR^{1D};
W₁ is hydrogen, optionally substituted C₁-C₆ alkyl, —NR^{1E}—C(O)—NR^{1E}R^{1F}, —C(O)—NR^{1E}R^{1F}, or (AA)₁₋₁₀;
W² is hydrogen, optionally substituted C₁-C₆ alkyl, —C(O)—NR^{1E}R^{1F}, or (AA)₁₋₁₀;
each R^{1D} and R^{1E} is independently hydrogen or C₁-C₆ alkyl;
R^{1F} is hydrogen, an optionally substituted C₁-C₁₀ alkyl, C₁-C₁₀ heteroalkyl, PEG₁₋₂₀, or one or more AA; and
each AA is an amino acid residue selected from β-alanine, lysine, and arginine.

Aspect 5. The transcription modulator molecule of Aspect 4, wherein the first terminus comprises a structure of Formula (A-3), or a pharmaceutically acceptable salt thereof:

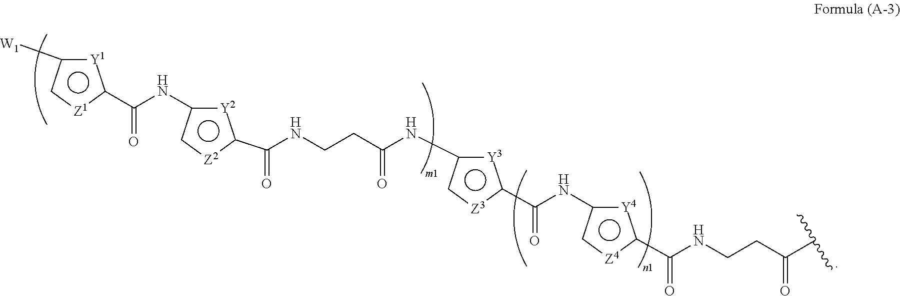

Aspect 6. The transcription modulator molecule of Aspect 4 or 5, or a pharmaceutically acceptable salt thereof, wherein each Z¹, Z², Z³, and Z⁴ is independently NR^{1D}, wherein R^{1D} is C₁-C₆ alkyl.

Aspect 7. The transcription modulator molecule of Aspect 6, or a pharmaceutically acceptable salt thereof, wherein each Z¹, Z², Z³, and Z⁴ is independently NCH₃.

Aspect 8. The transcription modulator molecule of Aspect 4 or 5, or a pharmaceutically acceptable salt thereof, wherein each Y¹ and Y³ are N; and each Y² and Y⁴ are independently CH or N.

Aspect 9. The transcription modulator molecule of Aspect 8, or a pharmaceutically acceptable salt thereof, wherein each Y² and Y⁴ is CH.

Aspect 10. The transcription modulator molecule of Aspect 4 or 5, wherein the first terminus comprises a structure of Formula (A-4), or a pharmaceutically acceptable salt thereof:

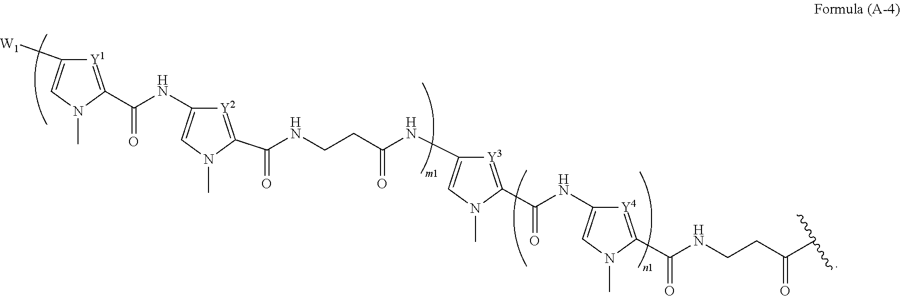

Aspect 11. The transcription modulator molecule of any one of Aspect 4-10, or a pharmaceutically acceptable salt thereof, wherein W₁ is optionally substituted C₁-C₆ alkyl, or —C(O)—NR^{1E}R^{1F}

Aspect 12. The transcription modulator molecule of Aspect 12, or a pharmaceutically acceptable salt thereof, wherein W₁ is —C(O)—NR^{1E}R^{1F}, wherein R^{1E} is hydrogen; and R^{1F} is hydrogen, optionally substituted C₁-C₁₀ alkyl, or PEG₁₋₂₀.

Aspect 13. The transcription modulator molecule of any one of Aspects 4-10, or a pharmaceutically acceptable salt thereof, wherein W₁ is hydrogen.

Aspect 14. The transcription modulator molecule of any one of Aspects 4-13, wherein m₁ is 2 or 3; and n₁ is 0 or 1.

Aspect 15. The transcription modulator molecule of any one of Aspects 1-14, or a pharmaceutically acceptable salt thereof, wherein the linker has a length of less than about 50 Angstroms.

Aspect 16. The transcription modulator molecule of any one of Aspects 1-14, or a pharmaceutically acceptable salt thereof, wherein the linker has a length of about 15 to 40 Angstroms.

Aspect 17. The transcription modulator molecule of any one of Aspects 1-14, or a pharmaceutically acceptable salt thereof, wherein the linker comprises between 5 and 50 chain atoms.

Aspect 18. The transcription modulator molecule of any one of Aspects 1-14, or a pharmaceutically acceptable salt thereof, wherein the linker comprises a multimer having from 2 to 50 spacing moieties, and wherein the spacing moiety is independently selected from the group consisting of —((CR^{3a}R^{3b})ₓ—O)_{y}—, —((CR^{3a}R^{3b})ₓ—NR^{4a})_{y}—, —((CR^{3a}R^{3b})ₓ—CH═CH—(CR^{3a}R^{3b})ₓ—O)_{y}—, optionally substituted —C₁₁₋₁₂ alkyl, optionally substituted C₂₋₁₀ alkenyl, optionally substituted C₂₋₁₀ alkynyl, optionally substituted C₆₋₁₀ arylene, optionally substituted C₃₋₇ cycloalkylene, optionally substituted 5- to 10-membered heteroarylene, optionally substituted 4- to 10-membered heterocycloalkylene, an amino acid residue, —O—, —C(O)NR^{4a}—, —NR^{4a}C(O)—, —C(O)—, —NR^{4a}—, and any combinations thereof; wherein
each x is independently 2-4;
each y is independently 1-10;
each R^{3a} and R^{3b} are independently selected from hydrogen, optionally substituted alkyl, optionally substituted alkenyl, optionally substituted alkynyl, optionally substituted alkoxy, optionally substituted amino, carboxyl, carboxyl ester, acyl, acyloxy, acyl amino, amino acyl, optionally substituted alkylamide, sulfonyl, optionally substituted thioalkoxy, optionally substituted aryl, optionally substituted heteroaryl, optionally substituted cycloalkyl, and optionally substituted heterocyclyl; and
each R^{4a} is independently a hydrogen or an optionally substituted C₁₋₆ alkyl.

Aspect 19. The transcription modulator molecule of any one of Aspects 1-14, or a pharmaceutically acceptable salt thereof, wherein the linker comprises -(T¹-V¹)ₐ-(T²-V²)_{b}-(T³-V³)_{c}-(T⁴-V⁴)ₙ-(T⁵-V⁵)ₑ—,
wherein a, b, c, d and e are each independently 0 or 1, and where the sum of a, b, c, d and e is 1 to 5;
T¹, T², T³, T⁴ and T⁵ are each independently selected from an optionally substituted (C₁-C₁₂) alkylene, optionally substituted alkenylene, optionally substituted alkynylene, (EA)W, (EDA)ₘ, (PEG)ₙ, (modified PEG)n, (AA)ₚ, —(CR^{2a}OH)ₕ—, optionally substituted (C₆-C₁₀) arylene, optionally substituted C₃₋₇ cycloalkylene, optionally substituted 5- to 10 membered heteroarylene, optionally substituted 4- to 10-membered heterocycloalkylene, a disulfide, a hydrazine, a carbohydrate, a beta-lactam, and an ester;
each m, p, and w are independently an integer from 1 to 20;
n is an integer from 1 to 30;
h is an integer from 1 to 12;
EA has the following structure:

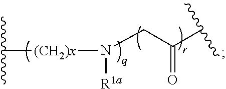

EDA has the following structure:

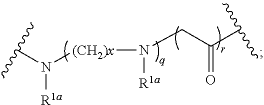

wherein each q is independently an integer from 1 to 6;
each x is independently an integer from 2 to 4 and
each r is independently 0 or 1; (PEG)n has the structure of —(CR^{2a}R^{2b}—CR^{2a}R^{2b}—O)—CR^{2a}R^{2b}_.
(modified PEG)ₙ has the structure of replacing at least one —(CR^{2a}R^{2b}—CR^{2a}R^{2b}—O)— in (PEG)n with —(CH₂—CR^{2a} ═CR^{2a}—CH₂—O)— or —(CR^{2a}R^{2b}—CR^{2a}R^{2b}—S)—;
AA is an amino acid residue;
V¹, V², V³, V⁴ and V⁵ are each independently selected from the group consisting of a bond, —CO—, —NR^{1a}—, —CONR^{1a}—, —NR^{1a}CO—, —CONR^{1a}C₁₋₄ alkyl-, and —NR^{1a}CO—C₁₋₄ alkyl-;
each R^{1a} is independently hydrogen or and optionally substituted C₁₋₆ alkyl; and each R^{2a} and R^{2b} are independently selected from hydrogen, alkyl, substituted alkyl, alkenyl, substituted alkenyl, alkynyl, substituted alkynyl, halogen, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl.

Aspect 20. The transcription modulator molecule of Aspect 19, or a pharmaceutically acceptable salt thereof, wherein T¹, T², T³, T⁴, and T⁵ are each independently selected from (C₁-C₁₂)alkyl, substituted (C₁-C₁₂)alkyl, (EA)_{W}, (EDA)ₘ, (PEG)ₙ, (modified PEG)n, (AA)ₚ, —(CR^{2a}OH)ₕ—, an optionally substituted phenyl, piperidin-4-amino (P4A), piperidine-3-amino, piperazine, pyrrolidin-3-amino, azetidine-3-amino, para-amino-benzyloxycarbonyl (PABC), meta-amino-benzyloxycarbonyl (MABC), para-amino-benzyloxy (PABO), meta-amino-benzyloxy (MABO), para-aminobenzyl, an acetal group, a disulfide, a hydrazine, a carbohydrate, a beta-lactam, an ester, (AA)ₚ-MABC-(AA)ₚ, (AA)ₚ-MABO-(AA)ₚ, (AA)ₚ-PABO-(AA)ₚ and (AA)ₚ-PABC-(AA)ₚ.

Aspect 21. The transcription modulator molecule of Aspect 20 or a pharmaceutically acceptable salt thereof, wherein T¹, T², T³, T⁴ and T⁵ are each independently selected from (C₁-C₁₂)alkyl, substituted (C₁-C₁₂)alkyl, (EA)_{w}, (EDA)ₘ, (PEG)ₙ, (modified PEG)ₙ, (AA)ₚ, —(CR^{2a}OH)ₕ—, optionally substituted (C₆-C₁₀) arylene, 4-10 membered heterocycloalkene, and optionally substituted 5-10 membered heteroarylene.

Aspect 22. The transcription modulator molecule of Aspect 20 or a pharmaceutically acceptable salt thereof, wherein T⁴ or T⁵ is an optionally substituted (C₆-C₁₀) arylene.

Aspect 23. The transcription modulator molecule of Aspect 20 or a pharmaceutically acceptable salt thereof, wherein T⁴ or T⁵ is an optionally substituted phenylene.

Aspect 24. The transcription modulator molecule of any one of Aspects 1-20 or a pharmaceutically acceptable salt thereof, wherein the linker comprises —N(R^{1a})(CH₂)ₓN(R^{1b})(CH₂)ₓN—, wherein R^{1a} and R^{1b} are each independently selected from hydrogen or optionally substituted C₁-C₆ alkyl; and each x is independently an integer in the range of 1-6.

Aspect 25. The transcription modulator molecule of any one of Aspects 1-20 or 24, or a pharmaceutically acceptable salt thereof, wherein the linker comprises —(CH₂—C(O)N(R″)—(CH₂)q-N(R′)—(CH₂)_{q}—N(R″)C(O)—(CH₂)ₓ—C(O)N(R″)-A-, —(CH₂)ₓ—C(O)N(R″)—(CH₂CH₂O)_{y}(CH₂)ₓ—C(O)N(R″)-A-, —C(O)N(R″)—(CH₂)_{q}—N(R′)—(CH₂)_{q}—N(R″)C(O)—(CH₂)ₓ-A-, —(CH₂)ₓ—O—(CH₂CH₂O)_{y}—(CH₂)ₓN(R″)C(O)—(CH₂)ₓ-A-, or —N(R″)C(O)—(CH₂)—C(O)N(R″)—(CH₂)ₓ—O(CH₂CH₂O)_{y}(CH₂)ₓ-A-; wherein R′ is methyl; R″ is hydrogen; each x and y are independently an integer from 1 to 10; each q is independently an integer from 2 to 10; and each A is independently selected from a bond, an optionally substituted C₁₋₁₂ alkyl, an optionally substituted C₆₋₁₀ arylene, optionally substituted C₃₋₇ cycloalkylene, optionally substituted 5- to 10-membered heteroarylene, and optionally substituted 4- to 10-membered heterocycloalkylene.

Aspect 26. The transcription modulator molecule of Aspect 25 or a pharmaceutically acceptable salt thereof, wherein the linker comprises —(CH₂CH₂—O)ₓ₁- or —(CH₂CH₂—O)ₓ₂-A²-(CH₂CH₂—O)ₓ₃—, wherein A² is an optionally substituted 4- to 10-membered heterocycloalkylene or spirocyclene, and each x1, x2, and x3 is independently an integer from 1-15.

Aspect 27. The transcription modulator molecule of Aspect 26 or a pharmaceutically acceptable salt thereof wherein A² is selected from

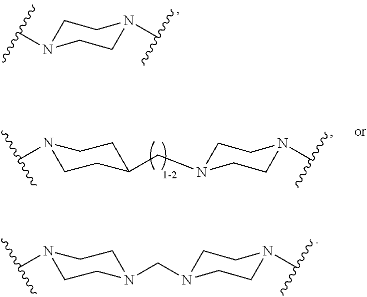

Aspect 28. The transcription modulator molecule of Aspect 26 or a pharmaceutically acceptable salt thereof, wherein A² comprises

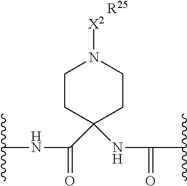

wherein,
X² is absent or —C(O)—; and
R¹⁵ is C₁₋₁₀ alkyl or C₁₋₁₀ heteroalkyl.

Aspect 29. The transcription modulator molecule of any one of Aspects 1-28, or a pharmaceutically acceptable salt thereof, wherein the linker is joined with the first terminus with a group selected from —CO—, —NR^{1a}—, C₁₋₁₂ alkyl, —CONR^{1a}—, and —NR^{1a}CO—; wherein each R^{1a} is independently a hydrogen or optionally substituted C₁₋₆ alkyl or optionally substituted —C₁₋₁₂ alkylene, optionally substituted C₂₋₁₀ alkenylene, optionally substituted C₂₋₁₀ alkynylene, optionally substituted C₆₋₁₀ arylene, optionally substituted C₃₇ cycloalkylene, optionally substituted 5- to 10-membered heteroarylene, and optionally substituted 4- to 10-membered heterocycloalkylene.

Aspect 30. The transcription modulator molecule of Aspect 29 or a pharmaceutically acceptable salt thereof, wherein the linker is joined with the second terminus with a group selected from optionally substituted 4- to 10-membered heterocycloalkylene.

Aspect 31. The transcription modulator molecule of Aspect of any one of Aspects 1-30, wherein the linker is joined with the second terminus comprises a structure of Formula (C-1), or a pharmaceutically acceptable salt thereof:

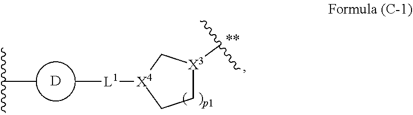

wherein,
Ring D is absent, arylene or heterocycloaklylene;
L¹ is absent, optionally substituted alkylene or alkenylene;
each X³ and X⁴ is independently CH or N;
p₁ is 0-3; and
** denotes attachment to the second terminus.

Aspect 32. The transcription modulator of Aspect 31, or a pharmaceutically acceptable salt thereof, wherein the Ring D is absent.

Aspect 33. The transcription modulator of Aspect 31 or a pharmaceutically acceptable salt thereof, wherein Ring D is 4 to 7-membered heterocyclene.

Aspect 34. The transcription modulator of any one of Aspects 31-33, or a pharmaceutically acceptable salt thereof, wherein p₁ is 0, 1, or 2

Aspect 35. The transcription modulator of any one of Aspects 31-34, or a pharmaceutically acceptable salt thereof, wherein X³ is N.

Aspect 36. The transcription modulator of any one of Aspects 31-35, or a pharmaceutically acceptable salt thereof, wherein L¹ is —(C^{R1G}R^{1G})ₓ-(alkylene)₂-(C^{R1G}R^{1G})_{y}—; wherein, x and y are each independently 0 or 1; and
each R^{1G} is hydrogen or C₁-C₃ alkyl.

Aspect 37. The transcription modulator molecule of Aspect 31, wherein the linker is joined with second terminus comprises a structure of Formula (C-2), or a pharmaceutically acceptable salt thereof:

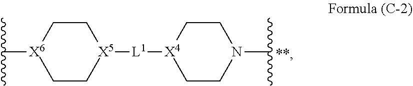

wherein each X⁵ and X⁶ is independently N or CH.

Aspect 38. The transcription modulator molecule of Aspect 37, or a pharmaceutically acceptable salt thereof, wherein each of X⁴ and X⁵ is independently N or CH; and X⁶ is N.

Aspect 39. The transcription modulator molecule of any one of Aspects 31-37, wherein L¹ is C₁-C₃ alkylene or C₁-C₃ alkenelene.

Aspect 40. The transcription modulator of Aspect 39, or a pharmaceutically acceptable salt thereof, wherein L¹ is —CH₂—, —CH₂CH₂—, —C≡C—, or —C≡C—C≡C—

Aspect 41. The transcription modulator molecule of Aspect of any one of Aspects 1-31, wherein the linker is joined with the second terminus comprises a structure of Formula (C-3), or a pharmaceutically acceptable salt thereof:

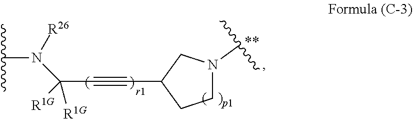

wherein,
p₁ is 0-3;
r₁ is 1-3;
R²⁶ is optionally substituted C₁₋₂₀ alkylene or heteroalkylene;
R^{1G} is hydrogen or C₁-C₃ alkyl; and
** denotes attachment to the second terminus.

Aspect 42. The transcription modulator molecule of any one of Aspects 31-41, or a pharmaceutically acceptable salt thereof, wherein the linker is joined with the second terminus with a group selected from:

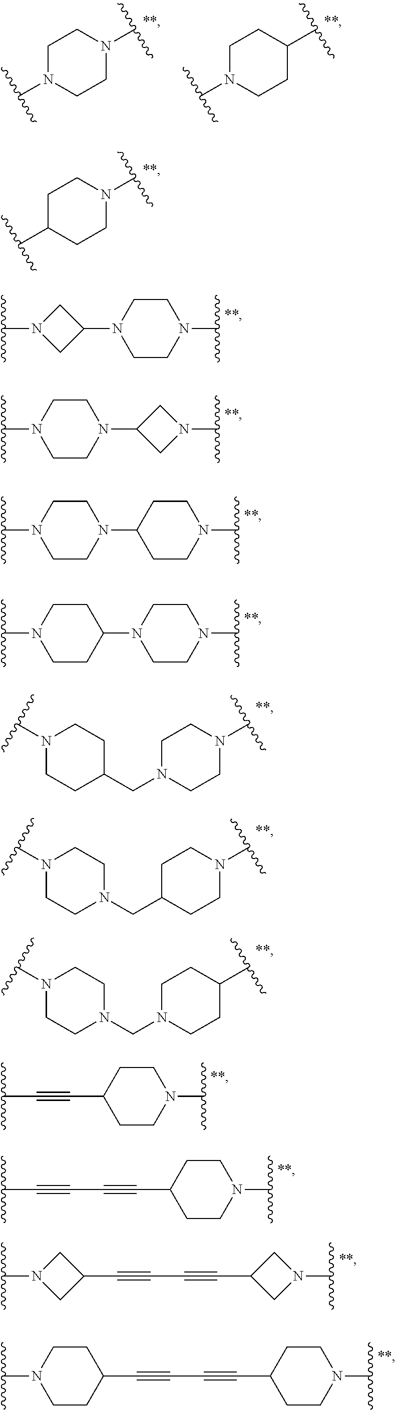

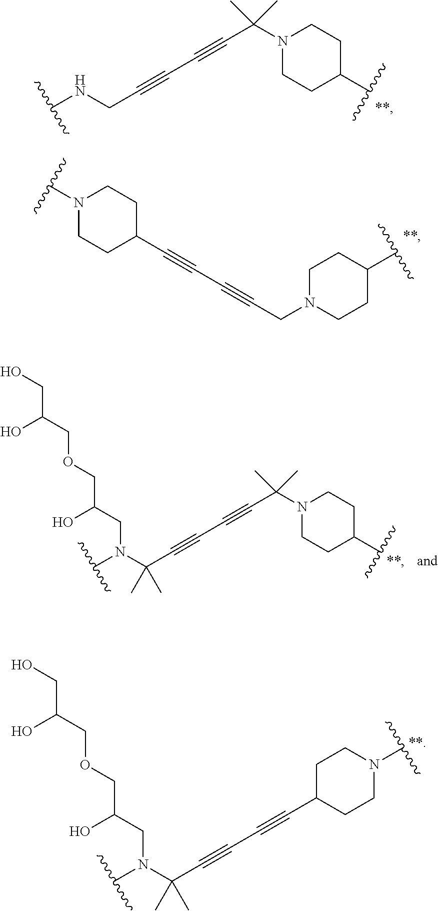 [SMILES: *NCC#CC#CC(C)(C)N1CCC(*)CC1 ; *C1CCN(CC#CC#CC2CCN(*)CC2)CC1 ; *C1CCN(C(C)(C)C#CC#CC(C)(C)N(*)CC(O)COCC(O)CO)CC1 ; *N1CCC(C#CC#CC(C)(C)N(*)CC(O)COCC(O)CO)CC1]

Aspect 43. The transcription modulator molecule of any one of Aspects 31-41, or a pharmaceutically acceptable salt thereof, wherein the linker is joined with the second terminus with a group selected from:

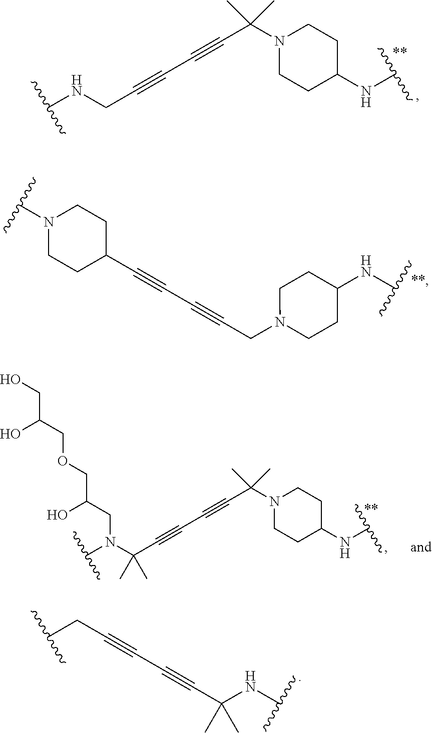 [SMILES: *NCC#CC#CC(C)(C)N1CCC(N*)CC1 ; *NC1CCN(CC#CC#CC2CCN(*)CC2)CC1 ; *NC1CCN(C(C)(C)C#CC#CC(C)(C)N(*)CC(O)COCC(O)CO)CC1 ; *CC#CC#CC(C)(C)N*]

Aspect 44. The transcription modulator molecule of any one of Aspects 1-43 or a pharmaceutically acceptable salt thereof, wherein the second terminus comprises a moiety capable of binding to the regulatory protein, and the moiety is from a compound capable of binding to the regulatory protein.

Aspect 45. The transcription modulator molecule of any one of Aspects 1-44 or a pharmaceutically acceptable salt thereof, wherein the second terminus comprises a moiety that binds to a bromodomain protein.

Aspect 46. The transcription modulator molecule of any one of Aspects 1-45, wherein the second terminus comprises a compound having the structure of Formula (9-A), or a pharmaceutically acceptable salt thereof:

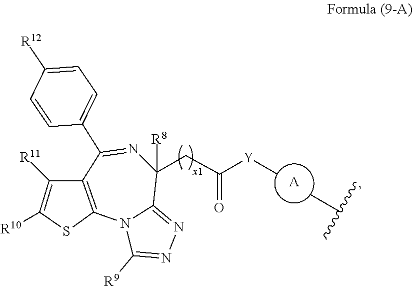

wherein,
Ring A is absent or 6-membered monocyclic aryl or heteroaryl;
Y is —NH— or —O—;
R⁹, R¹⁰, and R″ are each independently selected from hydrogen, optionally substituted C₁₋₆ alkyl, C₁₋₆ haloalkyl, or C₁₋₆ hydroxyalkyl;
R¹² is selected from hydrogen, halogen, —NO₂, —CN, optionally substituted aryl, optionally substituted C₁₋₆ alkyl, C₁₋₆ haloalkyl, or C₁₋₆ hydroxyalkyl;
or R¹² is —NR^{A}R^{B}, wherein
R^{A} and R^{B} are each independently hydrogen, optionally substituted C₁₋₆ alkyl or C₁₋₆ heteroalkyl; and
x₁ is an integer from 1-6.

Aspect 47. The transcription modulator molecule of Aspect 46, wherein the second terminus comprises a compound having the structure of Formula (9-B), or a pharmaceutically acceptable salt thereof:

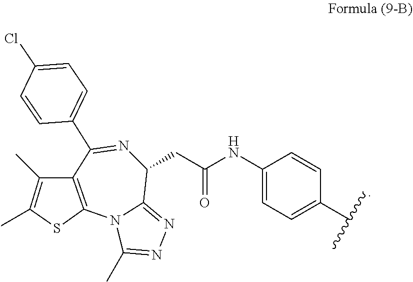 [SMILES: *c1ccc(NC(=O)C[C@H]2N=C(c3ccc(Cl)cc3)c3c(sc(C)c3C)-n3c(C)nnc32)cc1]

Aspect 48. The transcription modulator molecule of any one of Aspects 1-45, wherein the second terminus comprises a compound having the structure of Formula (10-A), or a pharmaceutically acceptable salt thereof:

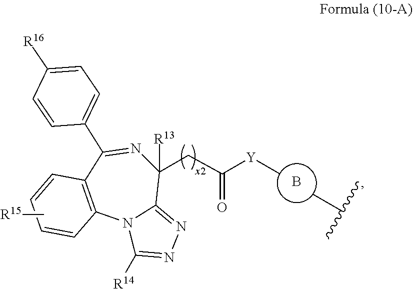

wherein,
Ring B is absent or 5-6-membered monocyclic aryl or heteroaryl or 4-8-membered heterocycle;
Y is —NH— or —O—;
R¹³ is selected from hydrogen or optionally substituted C₁-C₆ alkyl;
R¹⁴ and R¹⁵ are each independently selected from hydrogen, optionally substituted C₁₋₆ alkyl, C₁₋₆ haloalkyl, or C₁₋₆ hydroxyalkyl;
R¹⁶ is selected from hydrogen, halogen, —NO₂, —CN, optionally substituted aryl, optionally substituted C₁₋₆ alkyl, C₁₋₆ haloalkyl, or C₁₋₆ hydroxyalkyl;
or R¹⁶ is —NR^{A}R^{B}, wherein
R^{A} and R^{B} are each independently hydrogen, optionally substituted C₁₋₆ alkyl or C₁₋₆ heteroalkyl; and
x₂ is an integer from 1-6.

Aspect 49. The transcription modulator molecule of Aspect 48, wherein the second terminus comprises a compound having the structure of Formula (10-B), or a pharmaceutically acceptable salt thereof:

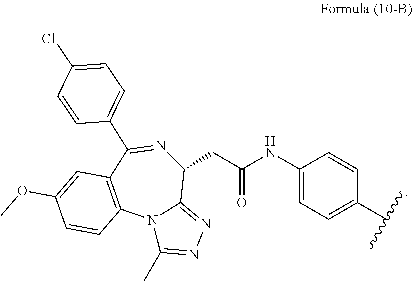 [SMILES: *c1ccc(NC(=O)C[C@H]2N=C(c3ccc(Cl)cc3)c3cc(OC)ccc3-n3c(C)nnc32)cc1]

Aspect 50. The transcription modulator molecule of any one of Aspects 1-45, wherein the second terminus comprises a compound having the structure of Formula (11-A), or a pharmaceutically acceptable salt thereof:

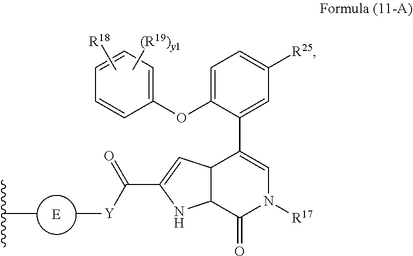

wherein,
Ring E is absent or 5-6-membered monocyclic aryl or heteroaryl or 4 to 8-membered heterocycle;
Y is —NH— or —O—;
R¹⁷ is hydrogen or —C₁-C₆ alkyl;
R¹⁸ and R¹⁹ are each independently hydrogen, halogen, —CN, —NO₂, optionally substituted —C₁-C₆ alkyl, C₁-C₆ haloalkyl or C₁-C₆ hydroxyalkyl;
or R¹⁸ is —NR^{A}R^{B}, wherein
R^{A} and R^{B} are each independently hydrogen, optionally substituted C₁₋₆ alkyl or C₁₋₆ heteroalkyl;
R²⁵ is optionally substituted optionally substituted C₁₋₆ alkyl, C₁-C₆ hydroxyalkyl, or —NHSO₂R^{A}; and
y₁ is 1-3.

Aspect 51. The transcription modulator molecule of Aspect 50, wherein the second terminus comprises a compound having the structure of Formula (11-B), or a pharmaceutically acceptable salt thereof:

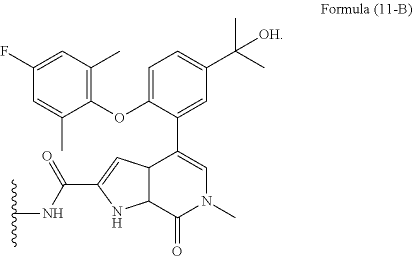 [SMILES: *NC(=O)C1=CC2C(c3cc(C(C)(C)O)ccc3Oc3c(C)cc(F)cc3C)=CN(C)C(=O)C2N1]

Aspect 52. The transcription modulator molecule of Aspect 50, wherein the second terminus comprises a compound having the structure of Formula (11-C), or a pharmaceutically acceptable salt thereof:

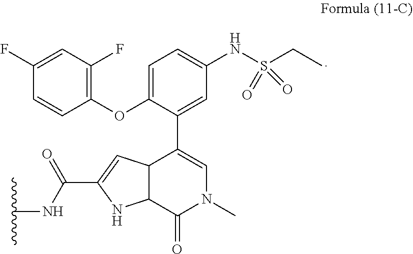 [SMILES: *NC(=O)C1=CC2C(c3cc(NS(=O)(=O)CC)ccc3Oc3ccc(F)cc3F)=CN(C)C(=O)C2N1]

Aspect 53. The transcription modulator molecule of any one of Aspects 1-45, wherein the second terminus comprises a compound having the structure of Formula (12-A), or a pharmaceutically acceptable salt thereof:

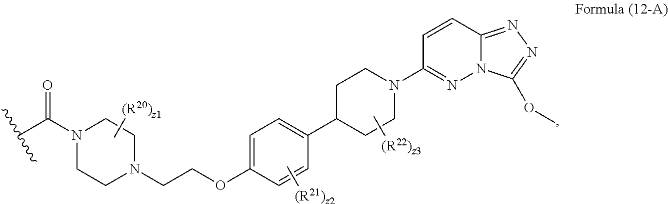

wherein,
each R²⁰, R²¹, and R²² is independently hydrogen, halogen, optionally substituted C₁-C₆ alkyl, C₁-C₆ haloalkyl or C₁-C₆ hydroxyalkyl; and
each z₁, z₂, and z₃ is independently 1-4.

Aspect 54. The transcription modulator molecule of Aspect 53, wherein the second terminus comprises a compound having the structure of Formula (12-B) or Formula (12-C), or a pharmaceutically acceptable salt thereof:

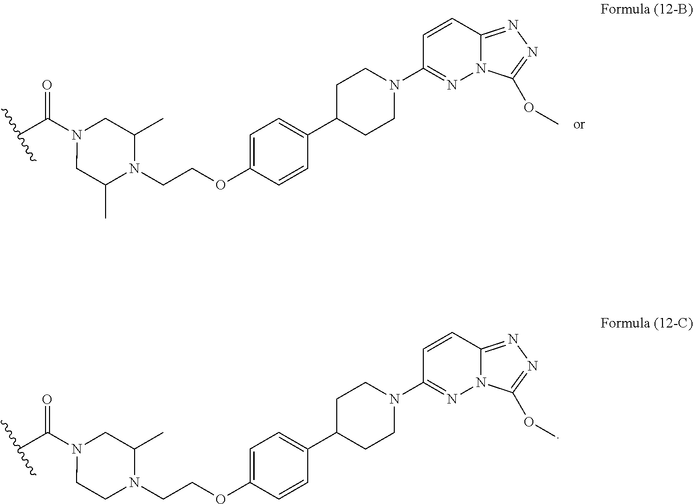

Aspect 55. The transcription modulator molecule of any one of Aspect 1-45, wherein the second terminus comprises a compound having the structure of Formula (13-A), or a pharmaceutically acceptable salt thereof:

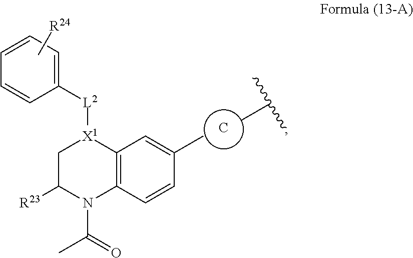

wherein,
Ring C is absent or monocyclic 6-membered aryl or heteroaryl;
X¹ is CH or N;
L² is —NR^{1D}— or —CR^{D}H—
R²³ is C₁-C₆ alkyl or C₃-C₆ cycloalkyl; and
R²⁴ is halogen, alkyl, hydroxyalkyl, haloalkyl; optionally substituted C₁-C₆ alkyl, C₁-C₆ haloalkyl or C₁-C₆ hydroxyalkyl; and
R^{D} is hydrogen or C₁₋₃ alkyl.

Aspect 56. The transcription modulator molecule of Aspect 55, wherein the second terminus comprises a compound having the structure of Formula (13-B), or a pharmaceutically acceptable salt thereof:

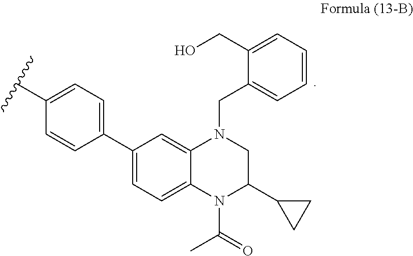 [SMILES: *c1ccc(-c2ccc3c(c2)N(Cc2ccccc2CO)CC(C2CC2)N3C(C)=O)cc1]

Aspect 57. The transcription modulator molecule of Aspect 55, wherein the second terminus comprises a compound having the structure of Formula (13-C), or a pharmaceutically acceptable salt thereof:

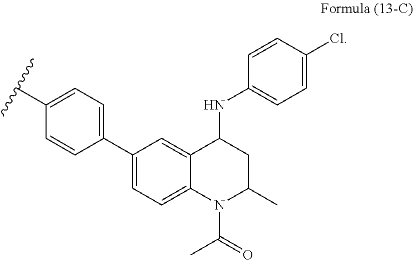 [SMILES: *c1ccc(-c2ccc3c(c2)C(Nc2ccc(Cl)cc2)CC(C)N3C(C)=O)cc1]

Aspect 58. The transcription modulator molecule of any one of Aspects 1-45, wherein the second terminus is

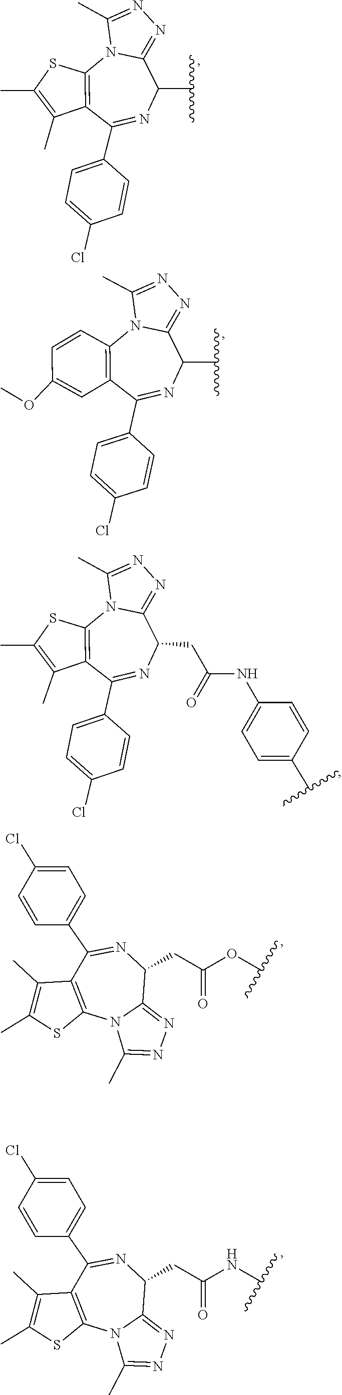

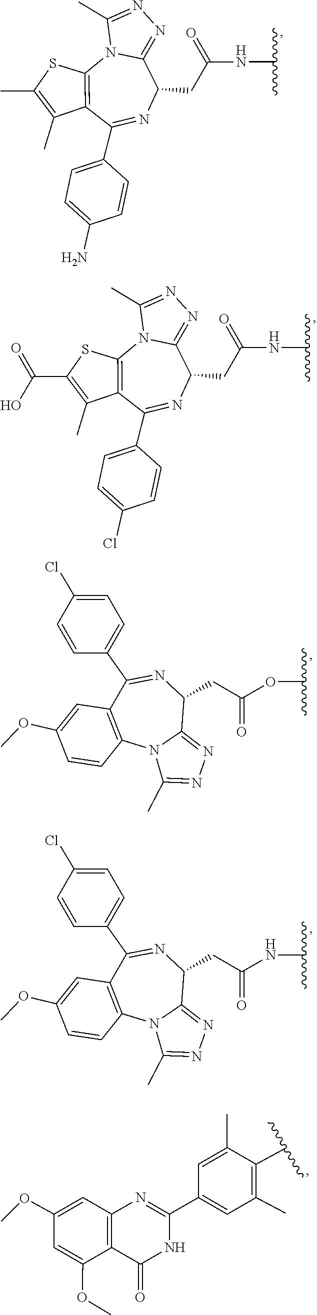

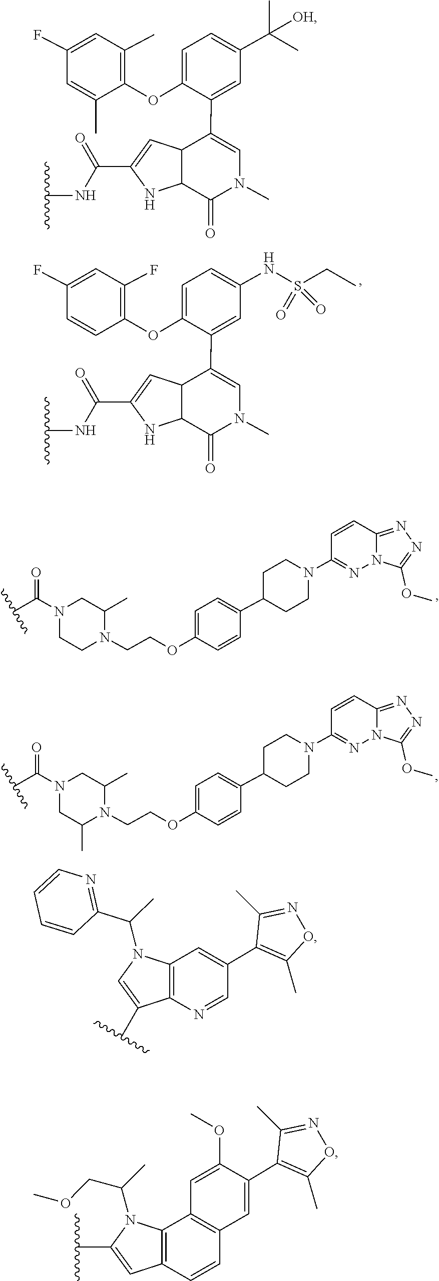

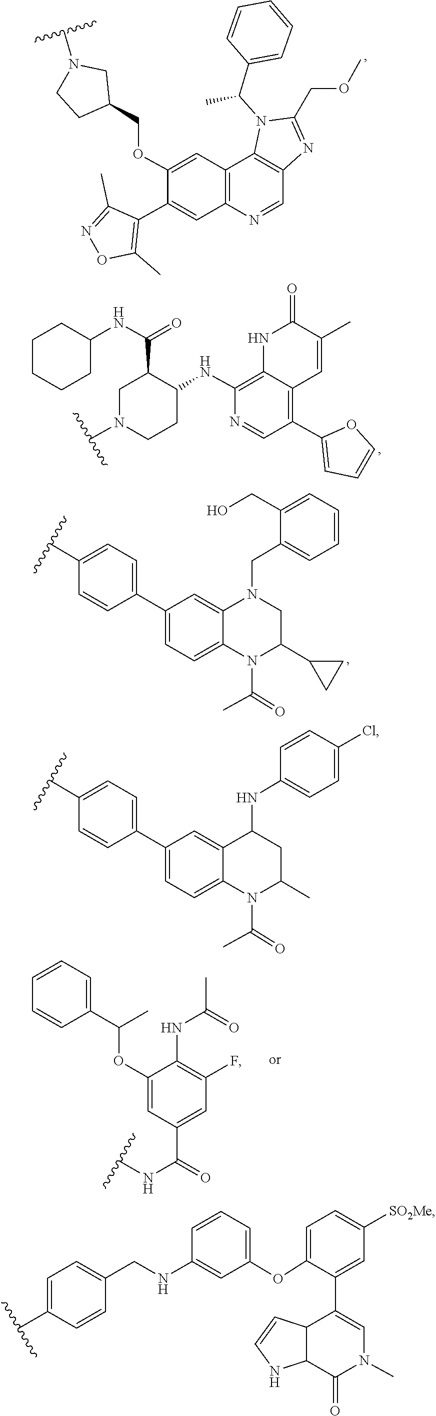

or a pharmaceutically acceptable salt thereof.

Aspect 59. A pharmaceutical composition comprising a transcription modulator molecule of any one of Aspects 1-58, or a pharmaceutically acceptable salt thereof and a pharmaceutically acceptable excipient.

Aspect 60. A method of modulation of the expression of fxn comprising contacting fxn with a transcription modulator molecule of any one of Aspects 1-58, or a pharmaceutically acceptable salt thereof.

Aspect 61. A method of treatment of a disease or condition caused by expression of a defective fxn in a patient in need thereof, comprising the administration of a therapeutically effective amount of a transcription modulator molecule of any one of Aspects 1-58, or a pharmaceutically acceptable salt thereof.

Aspect 62. The method as recited in Aspect 61, wherein the disease is Friedreich's ataxia (FA).

Aspect 63. A method of treating Friedreich's ataxia (FA) in a patient in need thereof, comprising administering to the patient a transcription modulator molecule of any one of Aspects 1-58, or a pharmaceutically acceptable salt thereof.

Aspect 64. The method of any one of Aspects 61-63, comprising administering a second therapeutic agent.

Aspect 65. The method of any one of Aspects 61-63, wherein the method comprises alleviating one or more of muscular atrophy, ataxia, fasciculation, or dementia.

What is claimed is:

1. A transcription modulator molecule having a first terminus, a second terminus, and an oligomeric backbone, wherein:
a) the first terminus comprises a DNA-binding moiety capable of noncovalently binding to a nucleotide repeat sequence GAA, wherein the first terminus has the structure of Formula (A-2), or a pharmaceutically acceptable salt thereof: 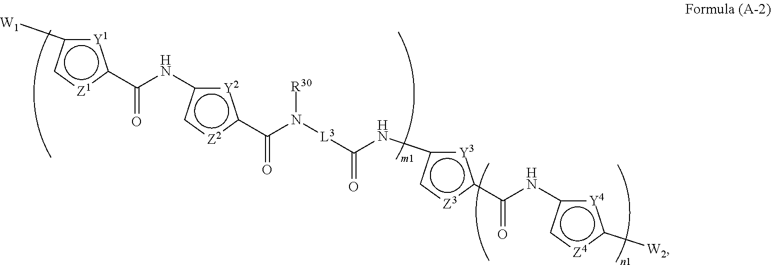
wherein;
m₁ is 1-4;
n₁ is 0-2;
each Y¹, Y², Y³, and Y⁴ is independently CH or N;
each Z¹, Z², Z³, and Z⁴ is independently O, S, or NR^{1D};
each L³ is optionally substituted C₁-C₆ alkylene, C₃-C₇ cycloalkylene, 3 to 7-membered heterocyclene, or 5 to 6-membered heteroarylene;
each R³⁰ is hydrogen or an C₁-C₆ alkyl; or
each R³⁰ and L³ join together with the atom(s) to which they are attached to form a 4- to 7-membered heterocyclic ring;
W₁ is hydrogen, an optionally substituted C₁-C₆ alkyl, —NR^{1E}—C(O)—NR^{1E}R^{1F}—C(O)—NR^{1E}R^{1F}, or (AA)₁₋₁₀;
W₂ is hydrogen, optionally substituted C₁-C₆ alkyl, —C(O)—NR^{1E}R^{1F}, or (AA)₁₋₁₀;
each R^{1D} and R^{1E} is independently hydrogen, optionally substituted C₁-C₅₀ alkyl, C₁-C₅₀ heteroalkyl, or PEG₁₅so;
R^{1F} is hydrogen, optionally substituted C₁-C₂₀ alkyl, C₁-C₂₀ heteroalkyl, PEG₁₋₂₀, or one or more AA; and
each AA is independently a naturally occurring amino acid;
b) the second terminus comprises a protein-binding moiety binding to a regulatory molecule that modulates an expression of a gene comprising the nucleotide repeat sequence GAA; and
c) the oligomeric backbone comprising a linker between the first terminus and the second terminus.

2. The transcription modulator molecule of claim 1, wherein the first terminus comprises a linear polyamide.

3. The transcription modulator molecule of claim 1 or 2, wherein the polyamide is capable of binding the DNA with an affinity of less than 500 nM.

4. The transcription modulator molecule of any one of claims 1-3, wherein the first terminus comprises a structure of Formula (A-3), or a pharmaceutically acceptable salt thereof: 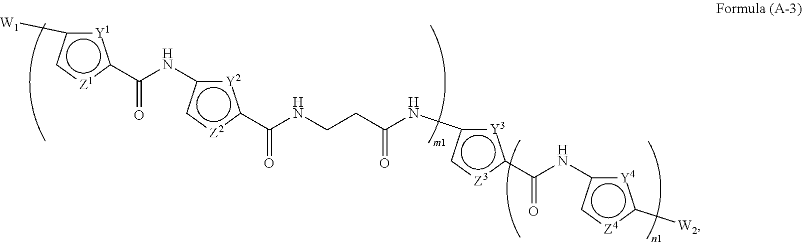
wherein;
m₁ is 1-4;
n₁ is 0-2;
each Y¹, Y², Y³, and Y⁴ is independently CH or N;
each Z¹, Z², Z³, and Z⁴ is independently O, S, or NR^{1D};
W₁ is hydrogen, optionally substituted C₁-C₆ alkyl, —NR^{1E}—C(O)—NR^{1E}R^{1F}, —C(O)—NR^{1E}R^{1F}, or (AA)₁₋₁₀;
W₂ is hydrogen, optionally substituted C₁-C₆ alkyl, —C(O)—NR^{1E}R^{1F}, or (AA)₁₋₁₀;
each R^{1D} and R^{1E} is independently hydrogen, optionally substituted C₁-C₅₀ alkyl, C₁-C₅₀ heteroalkyl, or PEG₁₋₅₀;
R^{1F} is hydrogen, optionally substituted C₁-C₂₀ alkyl, C₁-C₂₀ heteroalkyl, PEG₁₋₂₀, or one or more AA; and
each AA is independently an amino acid residue selected from β-alanine, lysine, and arginine.

5. The transcription modulator molecule of claim 4, wherein the first terminus comprises a structure of Formula (A-4), or a pharmaceutically acceptable salt thereof:

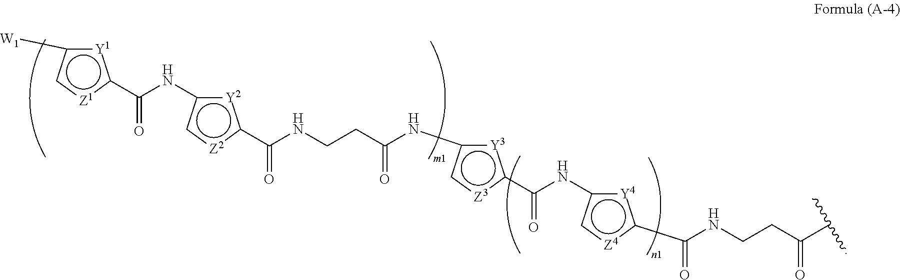

6. The transcription modulator molecule of any one of claims 1, 4, or 5, or a pharmaceutically acceptable salt thereof, wherein each Z¹, Z², Z³, and Z⁴ is independently NR^{1D}, wherein each R^{1D} is independently an optionally substituted C₁-C₆ alkyl.

7. The transcription modulator molecule of claim 6, or a pharmaceutically acceptable salt thereof, wherein each Z¹, Z², Z³, and Z⁴ is independently NCH₃.

8. The transcription modulator molecule of any one of claims 1 or 4-7, or a pharmaceutically acceptable salt thereof, wherein each Y¹ and Y³ are N; and each Y² and Y⁴ are each independently CH or N.

9. The transcription modulator molecule of claim 8, or a pharmaceutically acceptable salt thereof, wherein each Y² and Y⁴ are each CH.

10. The transcription modulator molecule of any one of claims 1 or 4-9, wherein the first terminus comprises a structure of Formula (A-5), or a pharmaceutically acceptable salt thereof:

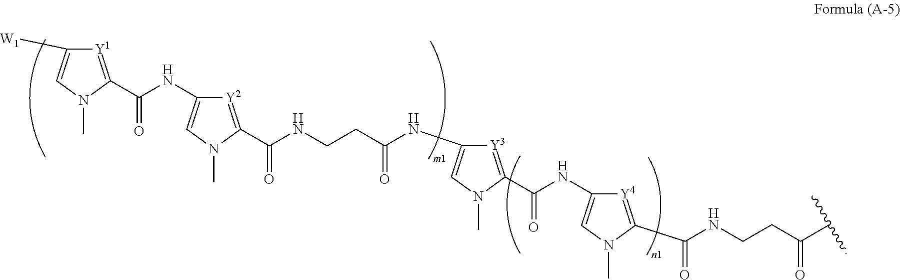

11. The transcription modulator molecule of any one of claim 1 or 4-9, or a pharmaceutically acceptable salt thereof, wherein W₁ is optionally substituted C₁-C₆ alkyl, or —C(O)—NR^{1E}R^{1F}.

12. The transcription modulator molecule of claim 11, or a pharmaceutically acceptable salt thereof, wherein W₁ is —C(O)—NR^{1E}R^{1F}, wherein R^{1E} is hydrogen; and R^{1F} is hydrogen, optionally substituted C₁-C₁₀ alkyl, or PEG₁₋₂₀.

13. The transcription modulator molecule of any one of claims 1 or 4-9, or a pharmaceutically acceptable salt thereof, wherein W₁ is hydrogen.

14. The transcription modulator molecule of any one of claims 1 or 4-13, wherein m₁ is 2 or 3; and n₁ is 0 or 1.

15. The transcription modulator molecule of any one of claims 1-14, or a pharmaceutically acceptable salt thereof, wherein the linker has a length of less than about 50 Angstroms.

16. The transcription modulator molecule of any one of claims 1-14, or a pharmaceutically acceptable salt thereof, wherein the linker has a length of about 15 to 40 Angstroms.

17. The transcription modulator molecule of any one of claims 1-14, or a pharmaceutically acceptable salt thereof, wherein the linker comprises between 5 and 50 chain atoms.

18. The transcription modulator molecule of any one of claims 1-14, or a pharmaceutically acceptable salt thereof, wherein the linker comprises a multimer having from 2 to 50 spacing moieties, and wherein the spacing moiety is independently selected from the group consisting of —((CR^{3a}R^{3b})ₓ—O)_{y}—, —((CR^{3a}R^{3b})ₓ—NR^{4a})_{y}—, —((CR^{3a}R^{3b})ₓ—CH═CH—(CR^{3a}R^{3b})ₓ—O)_{y}—, optionally substituted —C₁₋₁₂ alkyl, optionally substituted C₂-10 alkenyl, optionally substituted C₂₋₁₀ alkynyl, optionally substituted C₆₋₁₀ arylene, optionally substituted C₃₋₇ cycloalkylene, optionally substituted 5- to 10-membered heteroarylene, optionally substituted 4- to 10-membered heterocycloalkylene, an amino acid residue, —O—, —C(O)NR^{4a}—, —NR^{4a}C(O)—, —C(O)—, —NR^{4a}—, and any combinations thereof; wherein
each x is independently 2-4;
each y is independently 1-10;
each R^{3a} and R^{3b} are independently selected from hydrogen, optionally substituted alkyl, optionally substituted alkenyl, optionally substituted alkynyl, optionally substituted alkoxy, optionally substituted amino, carboxyl, carboxyl ester, acyl, acyloxy, acyl amino, amino acyl, optionally substituted alkylamide, sulfonyl, optionally substituted thioalkoxy, optionally substituted aryl, optionally substituted heteroaryl, optionally substituted cycloalkyl, and optionally substituted heterocyclyl; and
each R^{4a} is independently a hydrogen or an optionally substituted C₁₋₆ alkyl.

19. The transcription modulator molecule of any one of claims 1-14, or a pharmaceutically acceptable salt thereof, wherein the linker comprises -(T¹-V¹)ₙ-(T²-V²)_{b}-(T³-V³)_{c}-(T⁴-V⁴)ₙ-(T⁵-V⁵)ₑ—,
wherein a, b, c, d and e are each independently 0 or 1, and where the sum of a, b, c, d and e is 1 to 5;
T¹, T², T³, T⁴ and T⁵ are each independently selected from an optionally substituted (C₁-C₁₂) alkylene, optionally substituted alkenylene, optionally substituted alkynylene, (EA), (EDA)ₘ, (PEG)ₙ, (modified PEG)ₙ, (AA)ₚ, —(CR^{2a} OH)ₕ—, optionally substituted (C₆-C₁₀) arylene, optionally substituted C₃₋₇ cycloalkylene, optionally substituted 5- to 10 membered heteroarylene, optionally substituted 4- to 10-membered heterocycloalkylene, a disulfide, a hydrazine, a carbohydrate, a beta-lactam, and an ester;
each m, p, and w are independently an integer from 1 to 20;
n is an integer from 1 to 30;
h is an integer from 1 to 12;
EA has the following structure: 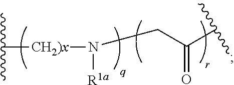
EDA has the following structure: 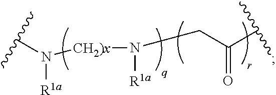
wherein each q is independently an integer from 1 to 6;
each x is independently an integer from 2 to 4 and
each r is independently 0 or 1; (PEG)ₙ has the structure of —(CR^{2a}R^{2b}—CR^{2a}R^{2b}—O)ₙ—CR^{2a}R^{2b}—;
(modified PEG)ₙ has the structure of replacing at least one —(CR^{2a}R^{2b}—CR^{2a}R^{2b}—O)— in (PEG)ₙ with —(CH₂—CR^{2a}═CR^{2a}—CH₂—O)— or —(CR^{2a}R^{2b}—CR^{2a}R^{2b}—S)—;
AA is an amino acid residue;
V¹, V², V³, V⁴ and V⁵ are each independently selected from the group consisting of a bond, —CO—, —NR^{1a}—, —CONR^{1a}—, —NR^{1a}CO—, —CONR^{1a}C₁₋₄ alkyl-, and —NR^{1a}CO—C₁₋₄ alkyl-;
each R^{1a} is independently hydrogen or and optionally substituted C₁₋₆ alkyl; and each R^{2a} and R^{2b} are independently selected from hydrogen, alkyl, substituted alkyl, alkenyl, substituted alkenyl, alkynyl, substituted alkynyl, halogen, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl.

20. The transcription modulator molecule of claim 19, or a pharmaceutically acceptable salt thereof, wherein T¹, T², T³, T⁴, and T⁵ are each independently selected from (C₁-C₁₂)alkyl, substituted (C₁-C₁₂)alkyl, (EA)_{w}, (EDA)ₘ, (PEG)ₙ, (modified PEG)ₙ, (AA)ₚ, —(CR^{2a} OH)ₕ—, an optionally substituted phenyl, piperidin-4-amino (P4A), piperidine-3-amino, piperazine, pyrrolidin-3-amino, azetidine-3-amino, para-amino-benzyloxycarbonyl (PABC), meta-amino-benzyloxycarbonyl (MABC), para-amino-benzyloxy (PABO), meta-amino-benzyloxy (MABO), para-aminobenzyl, an acetal group, a disulfide, a hydrazine, a carbohydrate, a beta-lactam, an ester, (AA)ₚ-MABC-(AA)ₚ, (AA)ₚ-MABO-(AA)ₚ, (AA)ₚ-PABO-(AA)ₚ and (AA)ₚ-PABC-(AA)ₚ.

21. The transcription modulator molecule of claim 20, or a pharmaceutically acceptable salt thereof, wherein T¹, T², T³, T⁴ and T⁵ are each independently selected from (C₁-C₁₂)alkyl, substituted (C₁-C₁₂)alkyl, (EA)_{W}, (EDA)ₘ, (PEG)ₙ, (modified PEG)ₙ, (AA)ₚ, —(CR^{2a}OH)ₕ—, optionally substituted (C₆-C₁₀) arylene, 4-10 membered heterocycloalkene, and optionally substituted 5-10 membered heteroarylene.

22. The transcription modulator molecule of claim 20 or 21, or a pharmaceutically acceptable salt thereof, wherein T⁴ or T⁵ is an optionally substituted (C₆-C₁₀) arylene.

23. The transcription modulator molecule of claim 22, or a pharmaceutically acceptable salt thereof, wherein T⁴ or T⁵ is an optionally substituted phenylene.

24. The transcription modulator molecule of any one of claims 1-23, or a pharmaceutically acceptable salt thereof, wherein the linker comprises —N(R^{1a})(CH₂)ₓN(R^{1b})(CH₂)ₓN—, wherein R^{1a} and R^{1b} are each independently selected from hydrogen or optionally substituted C₁-C₆ alkyl; and each x is independently an integer in the range of 1-6.

25. The transcription modulator molecule of any one of claims 1-20, or a pharmaceutically acceptable salt thereof, wherein the linker comprises —(CH₂—C(O)N(R″)—(CH₂)_{q}—N(R′)—(CH₂)_{q}—N(R″)C(O)—(CH₂)ₓ—C(O)N(R″)-A-, —(CH₂)ₓ—C(O)N(R″)—(CH₂CH₂O)_{y}(CH₂)ₙ—C(O)N(R″)-A-, —C(O)N(R″)—(CH₂)_{q} N(R′)—(CH₂)_{q}—N(R″)C(O)—(CH₂)ₓ-A-, —(CH₂)ₓ—O—(CH₂CH₂O)_{y}—(CH₂)ₓ—N(R″)C(O)—(CH₂)ₓ-A-, or —N(R″)C(O)—(CH₂)—C(O)N(R″)—(CH₂)ₓ—O(CH₂CH₂O)_{y}(CH₂)ₓ-A-; wherein R′ is methyl; R″ is hydrogen;
each x and y are independently an integer from 1 to 10; each q is independently an integer from 2 to 10; and
each A is independently selected from a bond, an optionally substituted C₁₋₁₂ alkyl, an optionally substituted C₆-10 arylene, optionally substituted C₃₋₇ cycloalkylene, optionally substituted 5- to 10-membered heteroarylene, and optionally substituted 4- to 10-membered heterocycloalkylene.

26. The transcription modulator molecule of claim 25, or a pharmaceutically acceptable salt thereof, wherein the linker comprises —(CH₂CH₂—O)Xi- or —(CH₂CH₂—O)ₓ₂-A²-(CH₂CH₂—O)X₃—, wherein A² is an optionally substituted 4- to 10-membered heterocycloalkylene or spirocyclene, and each x1, x2, and x3 is independently an integer from 1-15.

27. The transcription modulator molecule of claim 26, or a pharmaceutically acceptable salt thereof, wherein A² is selected from

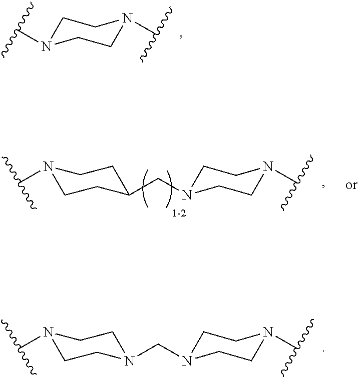

28. The transcription modulator molecule of claim 26, or a pharmaceutically acceptable salt thereof, wherein A² comprising 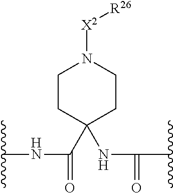
wherein,
X² is absent or —C(O)—; and
R²⁶ is an optionally substituted C₁₋₅₀ alkyl or C₁₋₅₀ heteroalkyl.

29. The transcription modulator molecule of any one of claims 1-28, or a pharmaceutically acceptable salt thereof, wherein the linker is joined with the second terminus with a group selected from —CO—, —NR^{1a}—, C₁₋₁₂ alkyl, —CONR^{1a}—, and —NR^{1a}CO—; wherein each R^{1a} is independently a hydrogen or optionally substituted C₁₋₆ alkyl or optionally substituted —C₁₋₁₂ alkylene, optionally substituted C₂₋₁₀ alkenylene, optionally substituted C₂₋₁₀ alkynylene, optionally substituted C₆₋₁₀ arylene, optionally substituted C₃₋₇ cycloalkylene, optionally substituted 5- to 10-membered heteroarylene, and optionally substituted 4- to 10-membered heterocycloalkylene.

30. The transcription modulator molecule of claim 29, or a pharmaceutically acceptable salt thereof, wherein the linker is joined with second terminus with a group selected from optionally substituted 4- to 10-membered heterocycloalkylene.

31. The transcription modulator molecule of claim of any one of claims 1-28, wherein the linker is joined with the second terminus with a moiety comprising a structure of Formula (C-1), or a pharmaceutically acceptable salt thereof: 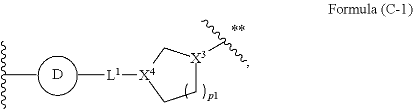
wherein,
Ring D is absent, or an arylene or heterocycloaklylene;
L¹ is absent, optionally substituted alkylene or alkenylene;
each X³ and X⁴ is independently CH or N;
p₁ is 0-3; and
** denotes attachment to the second terminus.

32. The transcription modulator of claim 31, or a pharmaceutically acceptable salt thereof, wherein the Ring D is absent.

33. The transcription modulator of claim 31, or a pharmaceutically acceptable salt thereof, wherein Ring D is 4 to 7-membered heterocyclene.

34. The transcription modulator of any one of claims 31-33, or a pharmaceutically acceptable salt thereof, wherein p₁ is 0, 1, or 2.

35. The transcription modulator of any one of claims 31-34, or a pharmaceutically acceptable salt thereof, wherein X³ is N.

36. The transcription modulator of any one of claims 31-35, or a pharmaceutically acceptable salt thereof, wherein L¹ is —(C^{R1G}R^{1G})ₓ-(alkylene)₂-(CR^{1G}R^{1G})_{y}—; wherein
x and y are each independently 0 or 1; and
each R^{1G} is hydrogen or C₁-C₃ alkyl.

37. The transcription modulator molecule of claim 31, wherein the linker is joined with the second terminus with a moiety comprising a structure of Formula (C-2) or a pharmaceutically acceptable salt thereof: 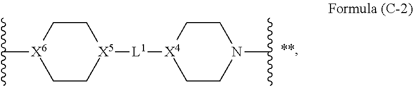
wherein each X⁵ and X⁶ is independently N or CH.

38. The transcription modulator molecule of claim 37, or a pharmaceutically acceptable salt thereof, wherein each of X⁴ and X⁵ is independently N or CH; and X⁶ is N.

39. The transcription modulator molecule of any one of claims 31-37, wherein L¹ is C₁-C₃ alkylene or C₁-C₃ alkenelene.

40. The transcription modulator of claim 39, or a pharmaceutically acceptable salt thereof, wherein L¹ is —CH₂—, —CH₂CH₂—, —C≡C—, or —C≡C—C≡C—.

41. The transcription modulator molecule of claim of any one of claims 1-31, wherein the linker is joined with the second terminus with a moiety comprising a structure of Formula (C-3), or a pharmaceutically acceptable salt thereof: 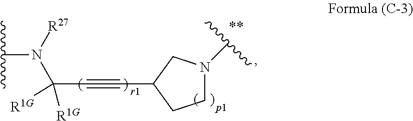
wherein,
p₁ is 0-3;
r₁ is 1-3;
R″ is an optionally substituted C₁₋₅₀ alkyl, C₁₋₅₀ heteroalkyl, —C(O)(C₁₋₅₀ alkyl), or —C(O)(C₁₋₅₀ heteroalkyl), wherein each alkyl and heteroalkyl is optionally substituted;
R^{1G} is hydrogen or C₁-C₃ alkyl; and
** denotes attachment to the second terminus.

42. The transcription modulator molecule of any one of claims 31-41, or a pharmaceutically acceptable salt thereof, wherein the linker is joined with the second terminus with a group selected from: 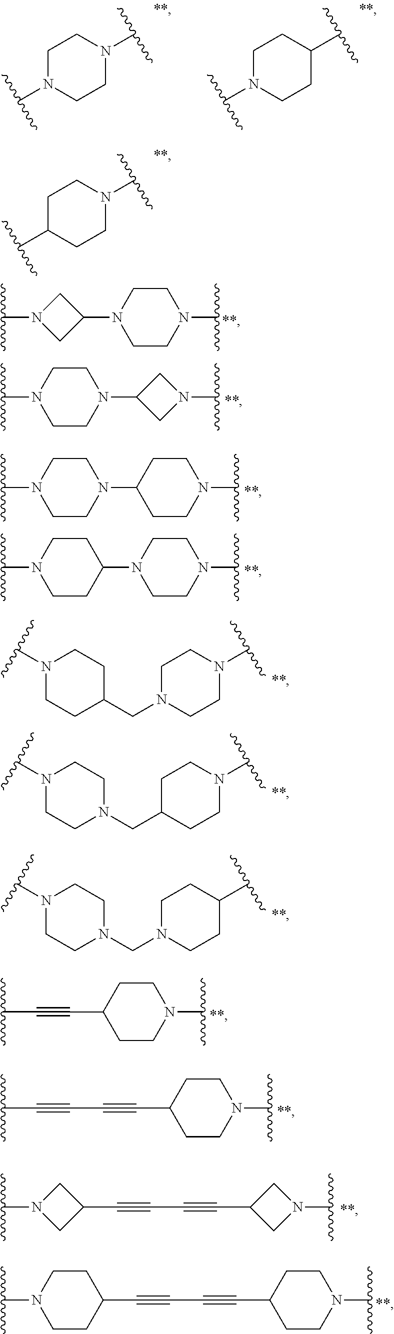 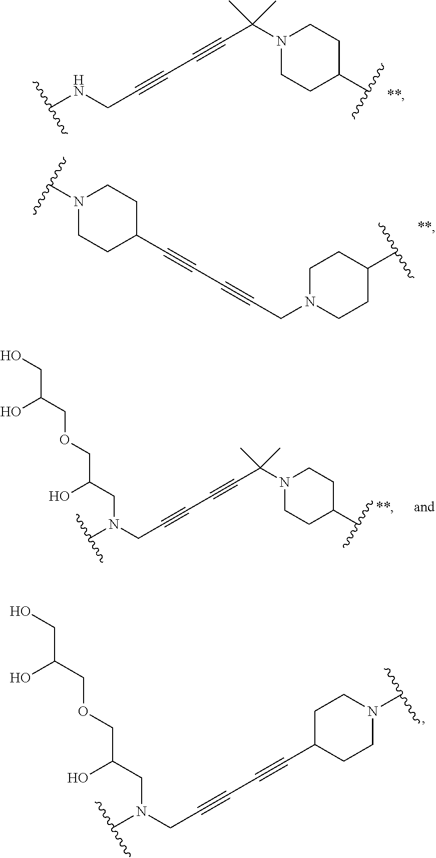 [SMILES: *NCC#CC#CC(C)(C)N1CCC(*)CC1 ; *C1CCN(CC#CC#CC2CCN(*)CC2)CC1 ; *C1CCN(C(C)(C)C#CC#CCN(*)CC(O)COCC(O)CO)CC1 ; *N1CCC(C#CC#CCN(*)CC(O)COCC(O)CO)CC1]
wherein ** denotes attachment to the second terminus.

43. The transcription modulator molecule of any one of claims 31-41, or a pharmaceutically acceptable salt thereof, wherein the linker is joined with the second terminus with a group selected from: 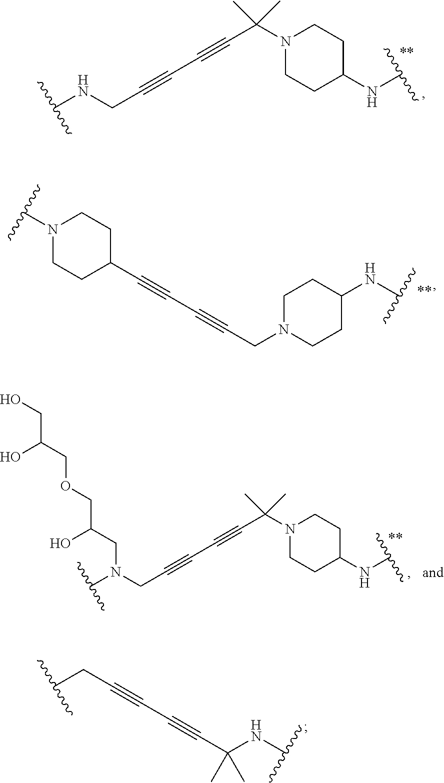 [SMILES: *NCC#CC#CC(C)(C)N1CCC(N*)CC1 ; *NC1CCN(CC#CC#CC2CCN(*)CC2)CC1 ; *NC1CCN(C(C)(C)C#CC#CCN(*)CC(O)COCC(O)CO)CC1 ; *CC#CC#CC(C)(C)N*]
wherein ** denotes attachment to the second terminus.

44. The transcription modulator molecule of any one of claims 1-43, or a pharmaceutically acceptable salt thereof, wherein the second terminus comprises a moiety capable of binding to a regulatory protein, and the moiety is from a compound capable of binding to a regulatory protein.

45. The transcription modulator molecule of any one of claims 1-44, or a pharmaceutically acceptable salt thereof, wherein the second terminus comprises a moiety that binds to a bromodomain protein.

46. The transcription modulator molecule of any one of claims 1-45, wherein the second terminus comprises a compound having the structure of Formula (9-A), or a pharmaceutically acceptable salt thereof: 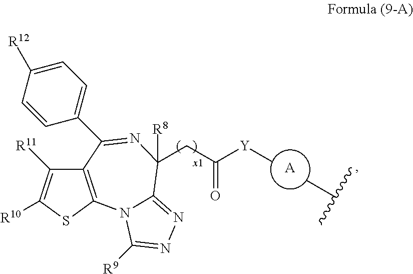
wherein,
Ring A is absent or an optionally substituted 6-membered monocyclic aryl or heteroaryl;
Y is —NH— or —O—;
R⁸ is hydrogen or C₁₋₆ alkyl;
R⁹, R¹⁰, and R″ are each independently selected from hydrogen, optionally substituted C₁₋₆ alkyl, C₁₋₆ haloalkyl, or C₁₋₆ hydroxyalkyl;
R¹² is selected from hydrogen, halogen, —NO₂, —CN, optionally substituted aryl, optionally substituted C₁₋₂₀ alkyl, C₁₋₂₀ heteroalkyl, C₁₋₆ haloalkyl, or C₁₋₆ hydroxyalkyl;
or R¹² is —NR^{A}R^{B}, wherein
R^{A} and R^{B} are each independently hydrogen, optionally substituted C₁₋₂₀ alkyl or C₁₋₂₀ heteroalkyl; and
x₁ is an integer from 1-6.

47. The transcription modulator molecule of claim 46, wherein the second terminus comprises a compound having the structure of Formula (9-B) or a pharmaceutically acceptable salt thereof:

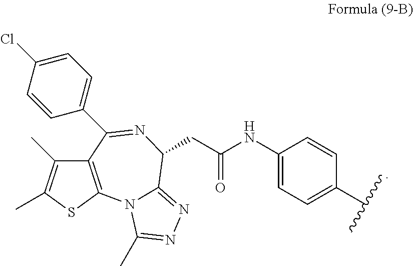 [SMILES: *c1ccc(NC(=O)C[C@H]2N=C(c3ccc(Cl)cc3)c3c(sc(C)c3C)-n3c(C)nnc32)cc1]

48. The transcription modulator molecule of any one of claims 1-45, wherein the second terminus comprises a compound having the structure of Formula (10-A), or a pharmaceutically acceptable salt thereof: 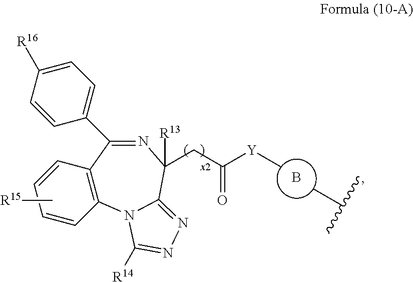
wherein,
Ring B is absent or an optionally substituted 5-6-membered monocyclic aryl or heteroaryl or 4-8-membered heterocycle;
Y is —NH— or —O—;
R¹³ is selected from hydrogen or optionally substituted C₁-C₆ alkyl;
R¹⁴ and R¹⁵ are each independently selected from hydrogen, optionally substituted C₁₋₆ alkyl, C₁₋₆ haloalkyl, or C₁₋₆ hydroxyalkyl;
R¹⁶ is selected from hydrogen, halogen, —NO₂, —CN, optionally substituted aryl, optionally substituted C₁₋₂₀ alkyl, C₁₋₂₀ heteroalkyl, C₁₋₆ haloalkyl, or C₁₋₆ hydroxyalkyl;
or R¹⁶ is —NR^{A}R^{B}, wherein
R^{A} and R^{B} are each independently hydrogen, optionally substituted C₁₋₂₀ alkyl or C₁₋₂₀ heteroalkyl; and
x₂ is an integer from 1-6.

49. The transcription modulator molecule of claim 48, wherein the second terminus comprises a compound having the structure of Formula (10-B), or a pharmaceutically acceptable salt thereof:

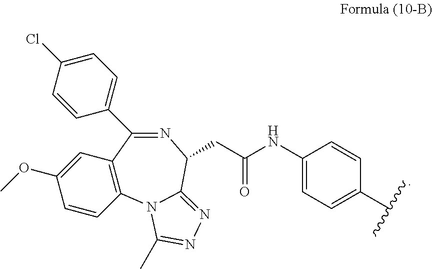 [SMILES: *c1ccc(NC(=O)C[C@H]2N=C(c3ccc(Cl)cc3)c3cc(OC)ccc3-n3c(C)nnc32)cc1]

50. The transcription modulator molecule of any one of claims 1-45, wherein the second terminus comprises a compound having the structure of Formula (11-A), or a pharmaceutically acceptable salt thereof: 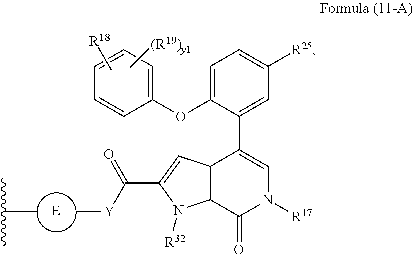
wherein,
Ring E is absent or an optionally substituted 5-6-membered monocyclic aryl or heteroaryl or 4-8-membered heterocycle;
Y is —NH— or —O—;
R¹⁷ is hydrogen or —C₁-C₆ alkyl;
R¹⁸ and R¹⁹ are each independently hydrogen, halogen, —CN, —NO₂, optionally substituted —C₁-C₆ alkyl, C₁-C₆ haloalkyl or C₁-C₆ hydroxyalkyl;
or R¹⁸ is —NR^{A}R^{B}, wherein
R^{A} and R^{B} are each independently hydrogen, optionally substituted C₁₋₆ alkyl or C₁₋₆ heteroalkyl;
R²⁵ is optionally substituted optionally substituted C₁₋₆ alkyl, C₁₋₆ heteroalkyl, C₁₋₆ alkenyl, C₁₋₆ alkynyl, C₁₋₆ hydroxyalkyl, or —NHSO₂R^{A};
R³² is hydrogen or an optionally substituted C₁₋₆ alkyl; and
y₁ is 1-3.

51. The transcription modulator molecule of claim 50, wherein the second terminus comprises a compound having the structure of Formula (11-B) or (11-C), or a pharmaceutically acceptable salt thereof:

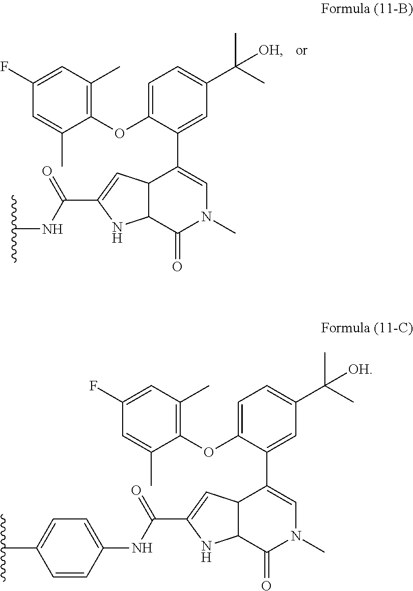 [SMILES: *NC(=O)C1=CC2C(c3cc(C(C)(C)O)ccc3Oc3c(C)cc(F)cc3C)=CN(C)C(=O)C2N1 ; *c1ccc(NC(=O)C2=CC3C(c4cc(C(C)(C)O)ccc4Oc4c(C)cc(F)cc4C)=CN(C)C(=O)C3N2)cc1]

52. The transcription modulator molecule of claim 50, wherein the second terminus comprises a compound having the structure of Formula (11-F) or a pharmaceutically acceptable salt thereof:

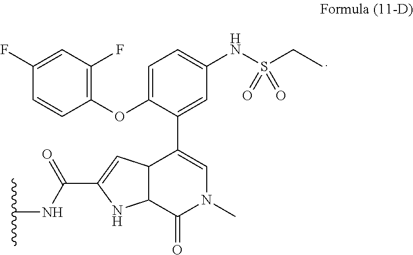 [SMILES: *NC(=O)C1=CC2C(c3cc(NS(=O)(=O)CC)ccc3Oc3ccc(F)cc3F)=CN(C)C(=O)C2N1]

53. The transcription modulator molecule of claim 50, wherein the second terminus comprises a compound having the structure of Formula (11-F), or a pharmaceutically acceptable salt thereof:

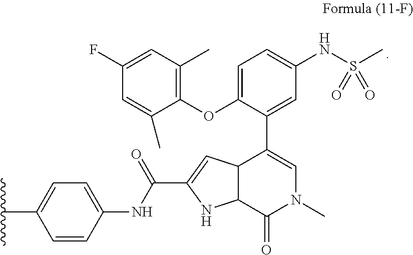 [SMILES: *c1ccc(NC(=O)C2=CC3C(c4cc(NS(C)(=O)=O)ccc4Oc4c(C)cc(F)cc4C)=CN(C)C(=O)C3N2)cc1]

54. The transcription modulator molecule of any one of claims 1-45, wherein the second terminus comprises a compound having the structure of Formula (12-A), or a pharmaceutically acceptable salt thereof: 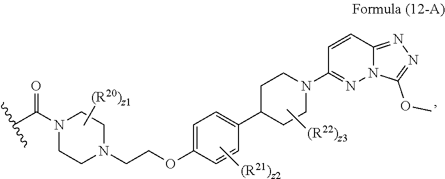
wherein,
each R²⁰, R²¹, and R²² is independently hydrogen, halogen, optionally substituted C₁-C₆ alkyl, C₁-C₆ haloalkyl or C₁-C₆ hydroxyalkyl; and
each z₁, z₂, and z₃ is independently 1-4.

55. The transcription modulator molecule of claim 54, wherein the second terminus comprises a compound having the structure of Formula (12-B) or Formula (12-C), or a pharmaceutically acceptable salt thereof:

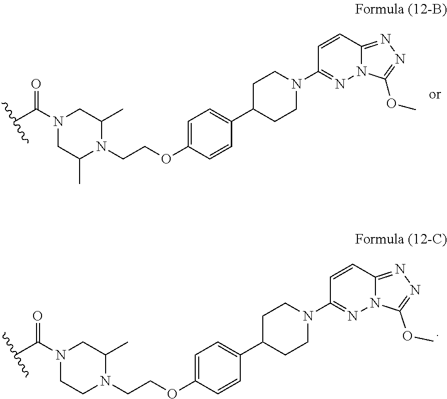 [SMILES: *C(=O)N1CC(C)N(CCOc2ccc(C3CCN(c4ccc5nnc(OC)n5n4)CC3)cc2)C(C)C1 ; *C(=O)N1CCN(CCOc2ccc(C3CCN(c4ccc5nnc(OC)n5n4)CC3)cc2)C(C)C1]

56. The transcription modulator molecule of any one of claim 1-45, wherein the second terminus comprises a compound having the structure of Formula (13-A), or a pharmaceutically acceptable salt thereof: 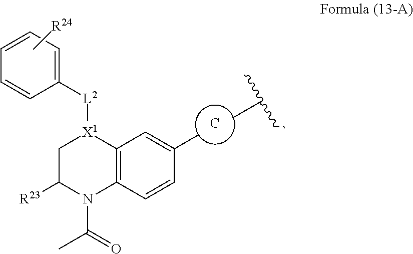
wherein,
Ring C is absent or an optionally substituted monocyclic 6-membered aryl or heteroaryl;
X¹ is CH or N;
L² is —NR^{1D}— or —CR^{D}H—
R²³ is C₁-C₆ alkyl or C₃-C₆ cycloalkyl; and
R²⁴ is halogen, alkyl, hydroxyalkyl, haloalkyl; optionally substituted C₁-C₆ alkyl, C₁-C₆ haloalkyl or C₁-C₆ hydroxyalkyl; and
R^{1D} is hydrogen or C₁₋₃ alkyl.

57. The transcription modulator molecule of claim 56, wherein the second terminus comprises a compound having the structure of Formula (13-B), or a pharmaceutically acceptable salt thereof:

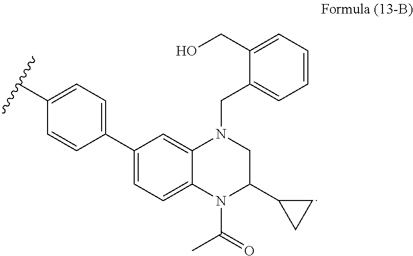 [SMILES: *c1ccc(-c2ccc3c(c2)N(Cc2ccccc2CO)CC(C2CC2)N3C(C)=O)cc1]

58. The transcription modulator molecule of claim 56, wherein the second terminus comprises a compound having the structure of Formula (13-C), or a pharmaceutically acceptable salt thereof:

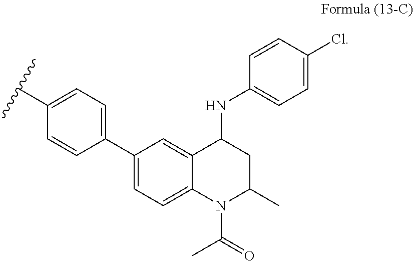 [SMILES: *c1ccc(-c2ccc3c(c2)C(Nc2ccc(Cl)cc2)CC(C)N3C(C)=O)cc1]

59. The transcription modulator molecule of any one of claims 1-45, wherein the second terminus is: 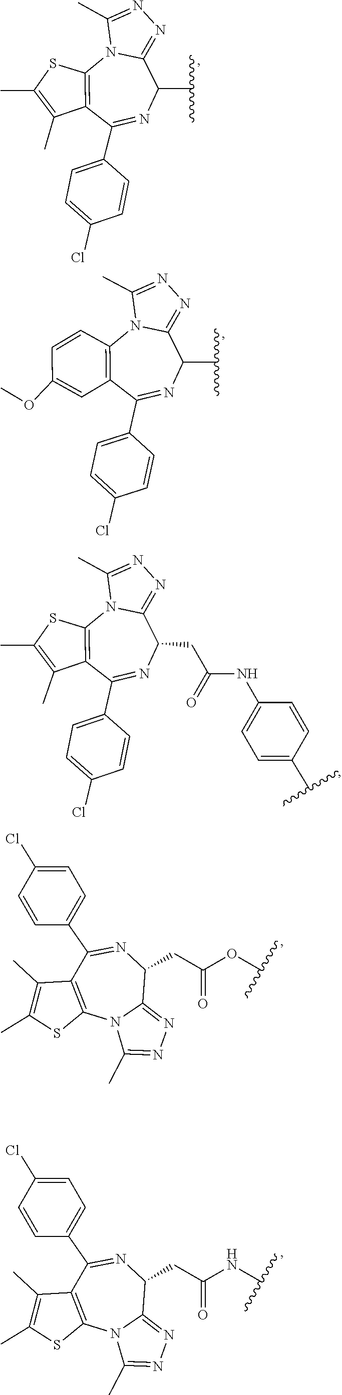 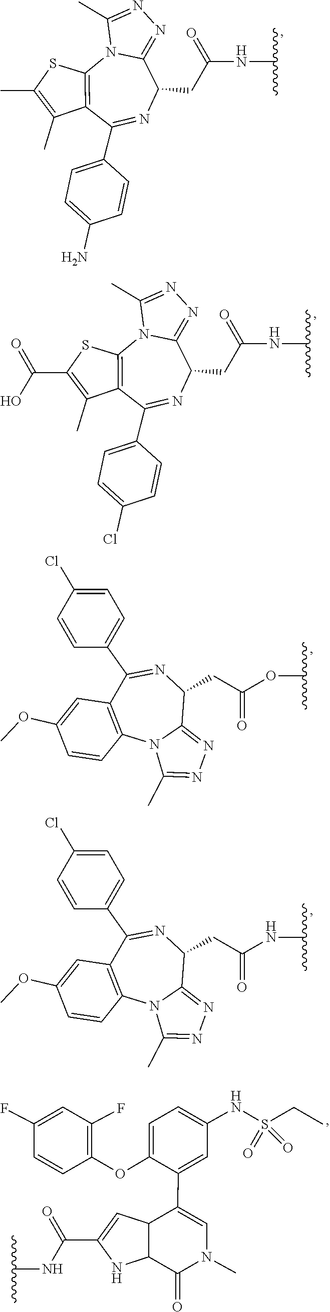 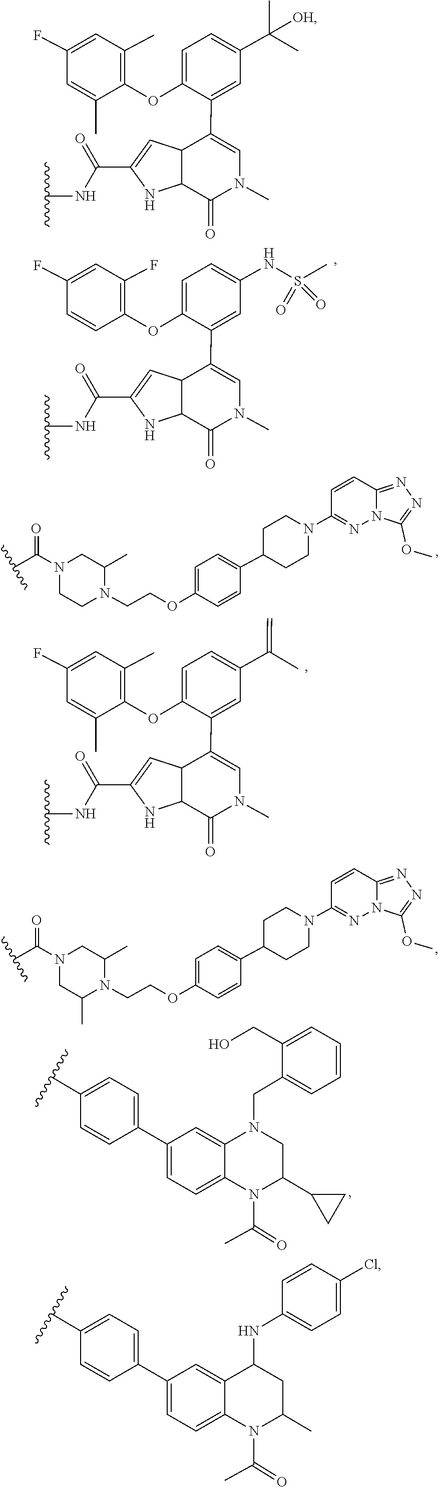 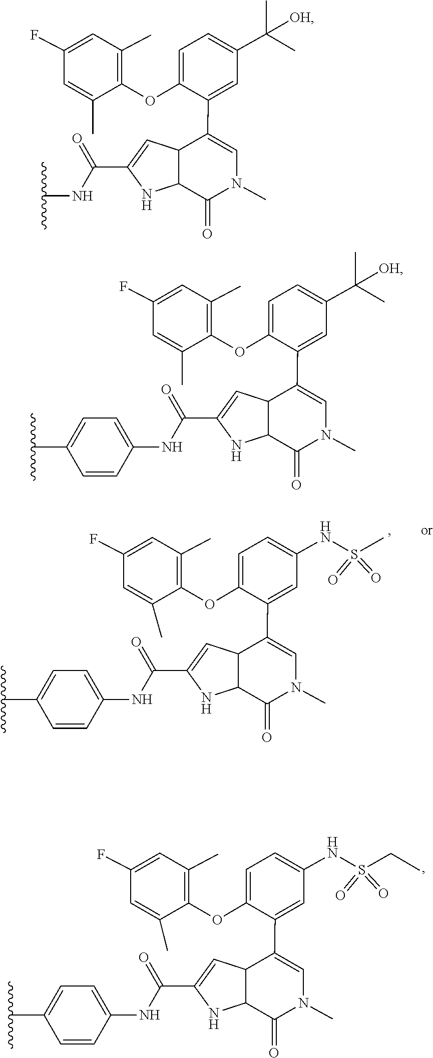 [SMILES: *NC(=O)C1=CC2C(c3cc(C(C)(C)O)ccc3Oc3c(C)cc(F)cc3C)=CN(C)C(=O)C2N1 ; *c1ccc(NC(=O)C2=CC3C(c4cc(C(C)(C)O)ccc4Oc4c(C)cc(F)cc4C)=CN(C)C(=O)C3N2)cc1 ; *c1ccc(NC(=O)C2=CC3C(c4cc(NS(C)(=O)=O)ccc4Oc4c(C)cc(F)cc4C)=CN(C)C(=O)C3N2)cc1 ; *c1ccc(NC(=O)C2=CC3C(c4cc(NS(=O)(=O)CC)ccc4Oc4c(C)cc(F)cc4C)=CN(C)C(=O)C3N2)cc1]
or a pharmaceutically acceptable salt thereof

60. The transcription modulator molecule of any one of claims 1-59, wherein the molecule is a compound disclosed in Table 3, or a pharmaceutically acceptable salt thereof.

61. A pharmaceutical composition comprising a transcription modulator molecule of any one of claims 1-60, or a pharmaceutically acceptable salt thereof; and a pharmaceutically acceptable excipient.

62. A method of modulation of the expression of fxn comprising contacting fxn with a transcription modulator molecule of any one of claims 1-60, or a pharmaceutically acceptable salt thereof or a pharmaceutical composition of claim 61.

63. A method of treating a disease or condition caused by expression of a defective fxn in a patient in need thereof, comprising administering to the patient therapeutically effective amount of a transcription modulator molecule of any one of claims 1-60, or a pharmaceutically acceptable salt thereof or a pharmaceutical composition of claim 61.

64. The method of claim 63, wherein the disease is Friedreich's ataxia (FA).

65. A method of treating Freidreich's ataxia (FA) in a patient in need thereof, comprising administering to the patient a transcription modulator molecule of any one of claims 1-60, or a pharmaceutically acceptable salt thereof.

66. The method of any one of claims 63-65, comprising administering a second therapeutic agent.

67. The method of any one of claims 63-65, wherein the method comprises alleviating one or more of muscular atrophy, ataxia, fasciculation, or dementia.
